(19) United States
(12) Patent Application Publication
(10) Pub. No.: US 2020/0255813 A1
(43) Pub. Date: Aug. 13, 2020

(54) PROSS OPTIMIZED ENZYMES

(71) Applicant: IGC BIO, INC., Cambridge, MA (US)

(72) Inventors: Lior ZIMMERMAN, Tel Aviv (IL); Dror BARAN, Tel Aviv (IL)

(73) Assignee: IGC BIO, INC., Cambridge, MA (US)

(21) Appl. No.: 16/629,372

(22) PCT Filed: Jul. 9, 2018
(86) PCT No.: PCT/US18/41278

§ 371 Date: Jan. 8, 2020

Related U.S. Application Data
(60) Provisional application No. 62/530,235, filed on Jul. 9, 2017.

(51) Int. Cl. C12N 9/22; C12N 15/113; C12N 15/85; C12N 15/82

(52) U.S. Cl. CPC C12N 9/22; C12N 15/113; C12N 15/8509; C12N 2310/20; C12N 15/8279; C12N 2800/80; C12N 15/8202

(57) ABSTRACT

The present invention provides enzymes that have been optimized by implementation of Protein Repair One Stop Shop (PROSS), an algorithm that generates protein design(s) for enhanced stability without changing either enzymatic properties or enzyme active site conformation of the respective enzyme. The protein design(s) generated by PROSS introduce mutations to the amino acid sequence of a wild-type protein, resulting in a mutated amino acid sequence that encodes a variant of the wild-type enzyme, i.e., an enzyme variant, which has an enhanced stability, core packing, surface polarity and backbone rigidity, a higher functional expression, and/or a combination thereof, compared to the stability core packing, surface polarity and backbone rigidity, functional expression and/or a combination thereof, of the wild-type enzyme.

### CROSS REFERENCE TO RELATED APPLICATIONS

This application claims benefit of U.S. Provisional Patent Application Ser. No. 62/530,235, filed Jul. 9, 2017; the priority date of which is hereby claimed.

### FIELD OF THE INVENTION

The present invention provides enzymes that have been optimized by implementation of Protein Repair One Stop Shop (PROSS), an algorithm that generates protein design(s) for enhanced stability without changing either enzymatic properties or enzyme active site conformation of the respective enzyme. The protein design(s) generated by PROSS introduce mutations to the amino acid sequence of a wild-type protein, resulting in a mutated amino acid sequence that encodes a variant of the wild-type enzyme, i.e., “an enzyme variant, ” which has an enhanced stability, core packing, surface polarity and backbone rigidity, a higher functional expression, and/or a combination thereof, compared to the stability, core packing, surface polarity and backbone rigidity, functional expression and/or a combination thereof of the wild-type enzyme.

### BACKGROUND OF THE INVENTION

Enzymes have been used in various manufacturing process, although indirectly, since the dawn of man. Isolated enzymes were first used in the 1914, and their large scale microbial production started in the 1960. The industrial enzyme business is steadily growing: in 2004, market cap for industrial enzymes was $2.5 billion with a predicted annual growth rate of 5%-10%, according to BioSpectrum, Industrial enzyme: High on radar, Mar. 15, 2006. And indeed, 11 years later it was valued at $5 billion, as reported by Global Industrial Enzymes Market-Segmented by Type, Application, and Geography-Trends and Forecasts (2017-2022), Mordor Intelligence, 2017.

In 2017, major applications of industrial enzymes are in medicine, textile industry, food and beverages industry and in recent times as analytical aids. Detergents (17%), food and feed (17%), leather and paper (17%) textiles (8%) and pharmaceutical (41%) are the major industries that use industrially produced enzymes, as described by Chandel et al., Industrial enzymes in bioindustrial sector development: An Indian perspective. J Comm Biotechnol 2007; 13:283-91.

As enzymes are used for more and more applications, there is a desire to use them in extreme environmental conditions, such as temperature, pH extremes and in the presence of salts, alkalis and surfactants. Major applications of enzymes are at high temperature (for example, washing 60 to 70° C., starch gelatinization 100° C., textile desizing 80 to 90° C., etc.), under high salt concentrations (in food industry), under alkaline conditions and in the presence of surfactants (for example, in detergents and in several biotransformation reaction systems among others). Thus, there is a need to engineer enzymes to be extremely stable, far beyond their wild-type environment.

Approaches to stabilize enzymes and protein in general are usually based on phylogenetic reconstruction of ancestral sequences' as discussed by Lehmann M., Pasamontes L., Lassen S. F., Wyss M., The consensus concept for thermostability engineering of proteins. Biochim. Biophys. Acta. 2000; 1543:408-415, and structure based rational or computational design, according to Borgo B., Havranek J. J., Automated selection of stabilizing mutations in designed and natural proteins, Proc. Natl. Acad. Sci. USA. 2012; 109: 1494-1499, and Jacak R., Leaver-Fay A., Kuhlman B., Computational protein design with explicit consideration of surface hydrophobic patches, Proteins. 2012; 80:825-838, and had some success in engineering proteins with improved stability and higher functional expression per Jacak.

Recently, Goldenzweig et al. developed PROSS—an automated method for protein stabilization—and demonstrated its robustness on 5 proteins from a wide spectrum of organisms, as described by Goldenzweig A, Goldsmith M, Hill S E, et al. Automated Structure- and Sequence-Based Design of Proteins for High Bacterial Expression and Stability. Mol Cell. 2016; 63(2):337-46, and by Campeotto I, Goldenzweig A, Davey J, Barfod L, Marshall J M, Silk S E, Wright K E, Draper S J, Higgins M K, Fleishman S J. One-step design of a stable variant of the malaria invasion protein RH5 for use as a vaccine immunogen. Proc Natl Acad Sci USA. 2017 Jan. 17. One of the targets was human acetylcholinesterase (hAChE), an enzyme mediating synaptic transmission, which was designed to have 2000-fold higher expression rate in E. coli compared to the wild-type and exhibited 20° C. higher thermostability with no change in enzymatic properties or in active site conformation as was determined by X-Ray crystallography.

In light of its extreme robustness, we applied PROSS to a number of industry relevant enzymes. Each iteration of PROSS resulted in an amino acid sequence of an enzyme with an improved stability.

### SUMMARY OF THE INVENTION

The present invention provides an alpha-amylase variant of a wild-type alpha-amylase from Bacillus amyloliquefaciens of SEQ ID NO: 1 comprising one or more mutations selected from K7N, L18M, V24I, T35S, N40S, V42D, A43N, A43T, N49D, T51N, T64K, Q65E, K68Q, R71A, Q91H, D93N, R97E, L102H, S107D, E120D, A127E, V135I, D147H, A149N, R157N, E194Q, T197E, R201G, F202Y, L203F, R205Q, L207V, N208A, P225T, S232G, R233N, T241N, T259A, S278E, S288A, S288K, S291A, S291E, D296S, S298P, D300S, T304L, I327L, I327M, G335A, S336A, S338A, G339S, E349Y, R356M, G359N, R366E, A369D, F371W, T377A, Q381K, Q406Y, S409N, T423N, T423D, and V424L.

The present invention provides an alpha-amylase variant of a wild-type alpha-amylase from Bacillus amyloliquefaciens of SEQ ID NO: 1 comprising mutations:
(a) K7N, L18M, V24I, T35S, N40S, V42D, A43N, N49D, T51N, T64K, Q65E, K68Q, R71A, Q91H, D93N, R97E, L102H, S107D, E120D, A127E, V135I, D147H, A149N, R157N, E194Q, T197E, R201G, F202Y, L203F, R205Q, L207V, N208A, P225T, S232G, R233N, T241N, T259A, S278E, S288A, S291E, D296S, S298P, D300S, T304L, I327L, G335A, S338A, G339S, E349Y, R356M, G359N, R366E, A369D, F371W, T377A, Q381K, Q406Y, S409N, T423N, and V424L;
(b) A43T, N49D, T51N, Q65E, R71A, Q91H, D93N, D147H, R233N, S291E, D296S, I327M, E349Y, R356M, R366E, A369D, T377A, and Q406Y;
(c) L18M, V42D, A43T, N49D, T51N, Q65E, R71A, Q91H, D93N, D147H, R157N, R233N, S291E, D296S, I327L, E349Y, R356M, G359N, R366E, A369D, T377A, Q406Y, S409N, and T423D;
(d) L18M, N40S, V42D, A43T, N49D, T51N, T64K, Q65E, R71A, Q91H, D93N, D147H, R157N, S232G, R233N, T241N, S288K, S291E, D296S, D300S, I327L, E349Y, R356M, G359N, R366E, A369D, T377A, Q381K, Q406Y, S409N, and T423D;
(e) L18M, N40S, V42D, A43T, N49D, T51N, T64K, Q65E, R71A, Q91H, D93N, A127E, D147H, A149N, R157N, P225T, S232G, R233N, T241N, T259A, S288K, S291E, D296S, D300S, I327L, G339S, E349Y, R356M, G359N, R366E, A369D, T377A, Q381K, Q406Y, S409N, and T423D;
(f) K7N, L18M, V24I, T35S, N40S, V42D, A43N, N49D, T51N, T64K, K68Q, R71A, Q91H, D93N, R97E, L102H, S107D, E120D, A127E, V135I, D147H, A149N, R157N, E194Q, T197E, R201G, F202Y, R205Q, N208A, P225T, S232G, R233N, T241N, T259A, S278E, S288A, S291A, D296S, S298P, D300S, T304L, I327L, G335A, S336A, S338A, G339S, E349Y, R356M, G359N, R366E, A369D, F371W, T377A, Q381K, Q406Y, S409N, T423N, and V424L;
(g) K7N, L18M, V24I, T35S, N40S, V42D, A43N, N49D, T51N, T64K, K68Q, R71A, Q91H, D93N, R97E, L102H, S107D, E120D, A127E, V135I, D147H, A149N, R157N, E194Q, T197E, R201G, F202Y, R205Q, N208A, P225T, S232G, R233N, T241N, T259A, S278E, S288A, S291A, D296S, S298P, D300S, T304L, I327L, G335A, S336A, S338A, G339S, E349Y, R356M, G359N, R366E, A369D, F371W, T377A, Q381K, Q406Y, S409N, T423N, and V424L;
(h) A43T, N49D, T51N, Q65E, R71A, Q91H, D93N, D147H, R233N, S291E, D296S, I327L, E349Y, R356M, R366E, A369D, T377A, and Q406Y; or
(i) A43T, N49D, T51N, Q65E, R71A, Q91H, D93N, D147H, R233N, S291E, D296S, I327L, A369D, T377A, and Q406Y.

The present invention also provides an alpha-amylase variant of a wild-type alpha-amylase from Bacillus licheniformis of SEQ ID NO: 2 comprising one or more mutations selected from K48H, K48Q, S49N, L63A, K64A, K64Q, H65Q, D72E, K87H, Y100W, S107K, Q109S, Q109T, R113N, Y114Q, T117S, A127E, E129H, E130K, V133L, K134Q, K134R, V137A, W175R, S197Q, K200A, K200H, K200I, F202Y, E204N, T205D, A206C, N208S, F216Y, N229G, T241N, K281A, K281E, S286N, S291G, A294Q, A294W, S295N, S340G, V355T, Q374K, T381H, P390D, E392F, I402T, Q406R, I412F, V427T, S428A, S435R, R448N, and L456I.

The present invention also provides an alpha-amylase variant of a wild-type alpha-amylase from Bacillus licheniformis of SEQ ID NO: 2 comprising mutations:
(a) K48H, S49N, L63A, K64A, H65Q, D72E, K87H, Y100W, S107K, Q109T, R113N, Y114Q, T117S, V133L, K134Q, V137A, S197Q, K200I, E204N, T205D, A206C, T241N, K281A, S286N, S291G, A294Q, S295N, S340G, V355T, T381H, E392F, I402T, I412F, V427T, S428A, S435R, R448N, and L456I;
(b) K48H, S49N, L63A, H65Q, K87H, Y100W, S107K, Q109S, R113N, Y114Q, T117S, K134Q, V137A, S197Q, K200H, E204N, T205D, K281A, S286N, A294W, S295N, S340G, T381H, E392F, I412F, V427T, S428A, S435R, R448N, and L456I;
(c) L63A, H65Q, K87H, Y100W, S107K, Q109S, R113N, T117S, E129H, K134Q, V137A, S197Q, E204N, T205D, K281A, S286N, A294W, S295N, S340G, T381H, E392F, I412F, V427T, S428A, S435R, R448N, and L456I;
(d) K48H, S49N, L63A, K64Q, H65Q, D72E, K87H, Y100W, S107K, Q109S, R113N, Y114Q, T117S, A127E, E130K, V133L, K134Q, V137A, W175R, S197Q, K200I, F202Y, E204N, T205D, A206C, N208S, F216Y, N229G, T241N, K281E, S286N, S291G, A294Q, S295N, S340G, V355T, Q374K, T381H, P390D, E392F, I402T, I412F, V427T, S428A, S435R, R448N, and L456I;
(e) K48Q, L63A, H65Q, K87H, Y100W, S107K, Q109S, R113N, T117S, E129H, K134Q, V137A, S197Q, E204N, T205D, K281A, S286N, A294W, S295N, S340G, T381H, E392F, I412F, V427T, S428A, S435R, R448N, and L456I;
(f) K48H, S49N, L63A, K64A, H65Q, D72E, K87H, Y100W, S107K, Q109S, R113N, Y114Q, T117S, E129H, V133L, K134Q, V137A, S197Q, K200I, E204N, T205D, A206C, T241N, K281E, S286N, S291G, A294Q, S295N, S340G, V355T, T381H, I402T, Q406R, I412F, V427T, S428A, S435R, R448N, and L456I;
(g) K48H, S49N, L63A, H65Q, K87H, Y100W, S107K, Q109S, R113N, Y114Q, T117S, K134Q, V137A, S197Q, E204N, T205D, K281A, S286N, S295N, S340G, T381H, E392F, I402T, I412F, V427T, S428A, S435R, R448N, and L456I;
(h) K48H, L63A, H65Q, K87H, Y100W, S107K, Q109S, R113N, T117S, E129H, K134Q, V137A, S197Q, E204N, T205D, K281A, S286N, S295N, S340G, T381H, E392F, I412F, V427T, S428A, S435R, R448N, and L456I;
(i) V427T, S428A, S435R, R448N, and L456I;
(j) K48H, S49N, L63A, H65Q, K87H, Y100W, S107K, Q109S, R113N, Y114Q, T117S, K134Q, V137A, S197Q, E204N, T205D, K281A, S286N, A294W, S295N, S340G, T381H, E392F, I402T, I412F, V427T, S428A, S435R, R448N, and L456I;
(k) K48H, S49N, L63A, H65Q, K87H, Y100W, S107K, Q109S, R113N, Y114Q, T117S, K134Q, V137A, S197Q, K200I, E204N, T205D, K281A, S286N, A294W, S295N, S340G, V355T, T381H, E392F, I402T, I412F, V427T, S428A, S435R, R448N, and L456I;
(l) K48H, S49N, L63A, H65Q, K87H, Y100W, S107K, Q109S, R113N, Y114Q, T117S, K134R, V137A, S197Q, K200I, E204N, T205D, K281A, S286N, A294W, S295N, S340G, V355T, T381H, I402T, Q406R, I412F, V427T, S428A, S435R, R448N, and L456I;
(m) K48H, L63A, H65Q, K87H, Y100W, S107K, Q109S, R113N, T117S, K134Q, V137A, S197Q, E204N, T205D, K281A, S286N, A294W, S295N, S340G, T381H, E392F, I412F, V427T, S428A, S435R, R448N, and L456I;
(n) K48H, S49N, L63A, H65Q, K87H, Y100W, S107K, Q109T, R113N, Y114Q, T117S, K134R, V137A, S197Q, K200H, E204N, T205D, K281A, S286N, A294W, S295N, S340G, T381H, Q406R, I412F, V427T, S428A, S435R, R448N, and L456I; or
(o) K48H, S49N, L63A, K64Q, H65Q, D72E, K87H, Y100W, S107K, Q109S, R113N, Y114Q, T117S, A127E, E129H, E130K, V133L, K134Q, V137A, W175R, S197Q, K200A, F202Y, E204N, T205D, A206C, N208S, F216Y, N229G, T241N, K281E, S286N, S291G, A294Q, S295N, S340G, V355T, Q374K, T381H, P390D, I402T, Q406R, I412F, V427T, S428A, S435R, R448N, and L456I.

The present invention further provides an alpha amylase variant of a wild-type alpha amylase from Thermoactinomyces of SEQ ID NO: 17 comprising one or more mutations selected from L2N, F7Y, E9R, A10P, S13N, S19D, T21K, T21N, Q22H, R24V, L27I, A29T, V35K, L40H, A42G, E50Q, A55E, L56M, G58K, S62E, L72E, S76P, T87E, A93R, A103K, A157S, R178K, P180D, E183K, E184D, A197K, H201N, H201Y, L210F, A211E, A211K, A211Q, P220E, D227E, R253Q, Q257K, S276K, S279D, S279P, R280E, V288K, Q289H, A292H, F313Y, N328Y, A333D, R340K, S344E, G361N, G361P, G361R, M364Q, S379A, T386K, E388K, H390N, H390T, R393Q, A396E, A396H, T399N, A410N, G412A, A434D, A434E, K435R, R437K, L447M, T449V, L451C, A461E, A461T, A463G, T464P, E475D, K477E, G482D, G482E, E489Q, R492E, R492Q, H495K, R496E, R496K, L497H, A498K, S499A, S507T, H509Y, Q513T, L516I, V523Y, Q524E, R535S, G536D, K538E, L542F, S548T, E560K, and Q568Y.

The present invention further provides an alpha amylase variant of a wild-type alpha amylase from Thermoactinomyces of SEQ ID NO: 17 comprising mutations:
a) L2N, F7Y, E9R, A10P, S13N, S19D, T21N, Q22H, R24V, L27I, A29T, V35K, L40H, A42G, E50Q, A55E, L56M, G58K, S62E, L72E, S76P, T87E, A93R, A103K, A157S, R178K, P180D, E183K, E184D, A197K, H201N, L210F, A211K, P220E, D227E, R253Q, Q257K, S276K, S279P, R280E, V288K, Q289H, A292H, F313Y, N328Y, A333D, R340K, S344E, G361P, M364Q, S379A, T386K, E388K, H390T, R393Q, A396H, T399N, A410N, G412A, A434E, K435R, R437K, L447M, T449V, L451C, A461T, A463G, T464P, E475D, K477E, G482E, E489Q, R492E, H495K, R496K, L497H, A498K, S499A, S507T, H509Y, Q513T, V523Y, Q524E, R535S, G536D, K538E, L542F, S548T, E560K, and Q568Y;
b) F7Y, E9R, A10P, S13N, S19D, A29T, L40H, L56M, G58K, S62E, P180D, A197K, H201Y, A211K, P220E, R253Q, S279P, A333D, G361N, M364Q, T386K, T399N, A410N, G412A, L451C, A463G, G482E, R496E, L497H, A498K, S499A, V523Y, and G536D;
c) F7Y, E9R, A29T, L40H, G58K, P180D, A197K, H201Y, P220E, R253Q, S279P, G361N, M364Q, T386K, A396E, T399N, A410N, L451C, A463G, G482E, R496E, A498K, and G536D;
d) F7Y, E9R, A10P, S13N, S19D, A29T, L40H, A42G, L56M, G58K, S62E, P180D, A197K, H201Y, A211K, P220E, R253Q, S279D, N328Y, A333D, G361N, M364Q, T386K, R393Q, T399N, A410N, G412A, A434D, T449V, L451C, A463G, G482E, E489Q, R492E, R496K, L497H, A498K, S499A, L516I, V523Y, R535S, and G536D;
e) F7Y, E9R, A10P, S13N, S19D, A29T, L40H, L56M, G58K, S62E, P180D, A197K, H201Y, A211K, P220E, R253Q, A333D, G361N, M364Q, T386K, A396E, T399N, A410N, G412A, L451C, A463G, G482E, R496E, L497H, A498K, S499A, V523Y, and G536D;
f) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, L210F, A211Q, P220E, D227E, R253Q, Q257K, S276K, S279D, R280E, V288K, A292H, F313Y, N328Y, A333D, G361R, M364Q, S379A, T386K, H390T, R393Q, T399N, A410N, G412A, A434D, K435R, R437K, L447M, T449V, L451C, A461T, A463G, E475D, K477E, G482E, E489Q, R492Q, H495K, R496E, L497H, A498K, S499A, S507T, L516I, V523Y, Q524E, R535S, and G536D;
g) F7Y, E9R, A29T, L40H, L56M, G58K, S62E, P180D, A197K, H201Y, P220E, R253Q, S279P, G361N, M364Q, T386K, T399N, A410N, L451C, A463G, G482E, R496E, A498K, S499A, V523Y, and G536D;
h) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, A211Q, P220E, D227E, R253Q, Q257K, S276K, S279P, R280E, V288K, A292H, F313Y, N328Y, A333D, G361N, M364Q, S379A, T386K, H390T, R393Q, T399N, A410N, G412A, A434D, K435R, R437K, L447M, T449V, L451C, A461T, A463G, E475D, K477E, G482D, E489Q, R492E, H495K, R496K, L497H, A498K, S499A, S507T, L516I, V523Y, Q524E, R535S, and G536D;
i) F7Y, E9R, A10P, S13N, S19D, A29T, L40H, A42G, A55E, L56M, G58K, S62E, P180D, E183K, A197K, H201N, L210F, A211Q, P220E, R253Q, S276K, S279P, A292H, N328Y, A333D, G361N, M364Q, T386K, H390N, R393Q, T399N, A410N, G412A, A434D, T449V, L451C, A461E, A463G, G482E, E489Q, H495K, R496E, L497H, A498K, S499A, L516I, V523Y, Q524E, R535S, and G536D;
j) F7Y, E9R, A10P, S13N, S19D, A29T, L40H, A42G, A55E, L56M, G58K, S62E, P180D, E183K, A197K, H201N, A211K, P220E, R253Q, S276K, S279D, A292H, N328Y, A333D, G361N, M364Q, T386K, H390N, R393Q, T399N, A410N, G412A, A434D, T449V, L451C, A461E, A463G, G482E, E489Q, R492E, H495K, R496K, L497H, A498K, S499A, L516I, V523Y, Q524E, R535S, and G536D;
k) F7Y, E9R, A29T, L40H, G58K, P180D, A197K, H201Y, P220E, R253Q, G361N, M364Q, T386K, T399N, A410N, L451C, A463G, G482E, R496E, A498K, and G536D;
l) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, A211Q, P220E, D227E, R253Q, Q257K, S276K, S279P, R280E, V288K, A292H, F313Y, N328Y, A333D, G361N, M364Q, S379A, T386K, H390N, R393Q, T399N, A410N, G412A, A434D, K435R, R437K, L447M, T449V, L451C, A461T, A463G, E475D, K477E, G482E, E489Q, R492E, H495K, R496K, L497H, A498K, S499A, S507T, L516I, V523Y, Q524E, R535S, and G536D;
m) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, A211Q, P220E, D227E, R253Q, Q257K, S276K, S279P, R280E, V288K, A292H, F313Y, A333D, G361N, M364Q, S379A, T386K, H390T, R393Q, T399N, A410N, G412A, A434D, K435R, R437K, L447M, T449V, L451C, A461T, A463G, E475D, K477E, G482D, E489Q, R492E, H495K, R496K, L497H, A498K, S499A, S507T, L516I, V523Y, Q524E, R535S, and G536D;
n) L2N, F7Y, E9R, A10P, S13N, S19D, T21N, Q22H, R24V, L27I, A29T, V35K, L40H, A42G, E50Q, A55E, L56M, G58K, S62E, L72E, S76P, T87E, A93R, A103K, A157S, R178K, P180D, E183K, E184D, A197K, H201N, A211Q, P220E, D227E, R253Q, Q257K, S276K, S279D, R280E, V288K, Q289H, A292H, F313Y, N328Y, A333D, R340K, S344E, G361P, M364Q, S379A, T386K, E388K, H390T, R393Q, A396H, T399N, A410N, G412A, A434E, K435R, R437K, L447M, T449V, L451C, A461T, A463G, T464P, E475D, K477E, G482D, E489Q, R492Q, H495K, R496E, L497H, A498K, S499A, S507T, H509Y, Q513T, L516I, V523Y, Q524E, R535S, G536D, K538E, L542F, S548T, E560K, and Q568Y;
o) F7Y, E9R, A10P, S13N, S19D, A29T, L40H, A42G, L56M, G58K, S62E, P180D, A197K, H201Y, A211E, P220E, R253Q, S279D, N328Y, A333D, G361R, M364Q, T386K, R393Q, T399N, A410N, G412A, A434D, T449V, L451C, A463G, G482E, E489Q, R492E, R496K, L497H, A498K, S499A, L516I, V523Y, R535S, and G536D; or
p) F7Y, E9R, A29T, L40H, L56M, G58K, S62E, P180D, A197K, H201Y, P220E, R253Q, G361N, M364Q, T386K, A396E, T399N, A410N, L451C, A463G, G482E, R496E, A498K, S499A, V523Y, and G536D.

The present invention provides a beta amylase variant of a wild-type beta amylase from barley of SEQ ID NO: 3 comprising one or more mutations selected from Y9H, Y13F, L16M, A20S, A20T, R26G, G30R, A35N, V41A, V41K, V41R, E42N, A64K, A64W, A69G, A69Y, E75Q, L76M, N102F, N102Y, I105L, D111E, V112E, G113M, H125W, T127R, I130F, T134S, Q140L, F143L, S147T, A153Q, Y155F, T157H, T157R, K164R, L167F, V171L, V177I, A182C, S189F, Q192E, F198Y, I205Q, A213K, F215W, K216E, K216M, A221K, V222A, N231D, R242S, D248S, L253Q, A262Q, N266D, N266G, K270Q, D277S, D277Q, K281A, K281S, V282I, Y286C, A291S, I292A, I292V, K302G, V303T, T325A, R328A, K331A, R334H, R334N, A335C, S336T, S336V, S351Q, S351P, Q352E, M354L, S355C, Q362R, L365A, L365M, G368A, G368C, N374Q, P386R, P386S, T391Q, R394L, I401L, H409L, L411M, F412Y, N421D, Q422E, E425H, E425Q, Q427S, V430M, V430R, V430W, T434E, D437K, D437N, H440S, L443N, P444D, R445H, Y448K, P456N, I462M, S463P, M466D, I467A, I467E, P473L and E482S.

The present invention provides a beta amylase variant of a wild-type beta amylase from barley of SEQ ID NO: 3 comprising mutations:
a) Y9H, R26G, G30R, V41K, A64W, N102Y, T127R, I130F, S147T, T157H, V171L, I205Q, N231D, L253Q, N266G, K270Q, D277Q, Y286C, I292A, K302G, R328A, K331A, R334H, M354L, G368A, P386R, T391Q, R394L, L411M, N421D, D437N, P444D, S463P, and P473L;
b) Y9H, G30R, V41K, A64W, N102Y, T127R, I130F, S147T, L167F, V171L, N231D, N266G, K270Q, D277Q, Y286C, I292A, K302G, R328A, K331A, R334H, M354L, G368A, P386S, T391Q, R394L, L411M, N421D, E425H, D437N, P444D, S463P, and P473L;
c) Y9H, R26G, G30R, V41K, A64W, N102Y, I105L, T127R, I130F, S147T, T157H, V171L, V177I, I205Q, F215W, N231D, L253Q, K270Q, D277Q, Y286C, I292A, K302G, R328A, K331A, R334H, Q352E, M354L, S355C, G368A, P386S, T391Q, R394L, L411M, N421D, D437N, P444D, R445H, S463P, and P473L;
d) Y9H, Y13F, A20S, R26G, G30R, A35N, V41A, A64K, A69G, E75Q, L76M, N102Y, I105L, D111E, V112E, G113M, H125W, T127R, I130F, T134S, Q140L, F143L, S147T, A153Q, Y155F, T157H, K164R, L167F, V171L, V177I, A182C, S189F, Q192E, F198Y, I205Q, A213K, F215W, K216M, A221K, V222A, N231D, R242S, D248S, L253Q, A262Q, N266D, K270Q, D277S, V282I, Y286C, A291S, I292A, K302G, V303T, T325A, R328A, K331A, R334H, A335C, S336V, S351Q, Q352E, M354L, S355C, L365A, G368C, P386S, T391Q, R394L, I401L, H409L, L411M, F412Y, N421D, Q422E, E425Q, Q427S, V430R, T434E, D437N, H440S, L443N, P444D, R445H, Y448K, P456N, I462M, S463P, M466D, I467A, P473L, and E482S;
e) Y9H, A20S, R26G, G30R, A35N, V41A, A64K, A69G, E75Q, L76M, N102Y, I105L, D111E, V112E, G113M, H125W, T127R, I130F, T134S, Q140L, S147T, A153Q, Y155F, T157H, K164R, L167F, V171L, V177I, A182C, S189F, Q192E, F198Y, I205Q, A213K, F215W, K216E, A221K, V222A, N231D, R242S, D248S, L253Q, A262Q, N266D, K270Q, D277S, K281A, V282I, Y286C, A291S, I292A, K302G, V303T, T325A, R328A, K331A, R334N, A335C, S336V, S351Q, Q352E, M354L, S355C, L365A, G368C, P386S, T391Q, R394L, I401L, H409L, L411M, F412Y, N421D, Q422E, E425Q, Q427S, V430R, T434E, D437N, H440S, L443N, P444D, R445H, Y448K, P456N, I462M, S463P, M466D, I467A, P473L, and E482S;
f) Y9H, Y13F, R26G, G30R, A35N, V41A, A64K, A69Y, L76M, N102Y, I105L, D111E, V112E, G113M, T127R, I130F, S147T, Y155F, T157H, K164R, V171L, V177I, S189F, F198Y, I205Q, A213K, F215W, K216M, V222A, N231D, R242S, D248S, L253Q, A262Q, N266D, K270Q, D277S, K281A, V282I, Y286C, A291S, I292A, K302G, T325A, R328A, K331A, R334N, A335C, S336V, S351P, Q352E, M354L, S355C, Q362R, L365A, G368C, P386S, T391Q, R394L, H409L, L411M, N421D, Q422E, Q427S, V430M, T434E, D437K, H440S, L443N, P444D, R445H, Y448K, P456N, I462M, S463P, M466D, I467E, and P473L;
g) Y9H, A20T, G30R, V41K, A64W, N102Y, T127R, I130F, S147T, L167F, V171L, N231D, N266G, K270Q, Y286C, I292V, K302G, R328A, K331A, R334H, M354L, G368A, P386S, T391Q, R394L, L411M, N421D, D437N, P444D, S463P, and P473L;
h) Y13F, R26G, G30R, A35N, V41A, A64K, A69G, L76M, N102Y, I105L, D111E, V112E, G113M, T127R, I130F, S147T, Y155F, T157H, K164R, L167F, V171L, V177I, S189F, F198Y, I205Q, A213K, F215W, K216M, V222A, N231D, R242S, D248S, L253Q, A262Q, N266D, K270Q, D277S, K281S, V282I, Y286C, A291S, I292A, K302G, T325A, R328A, K331A, R334H, A335C, S336V, S351P, Q352E, M354L, S355C, Q362R, L365A, G368C, P386R, T391Q, R394L, H409L, L411M, N421D, Q422E, E425Q, Q427S, V430W, T434E, D437N, H440S, L443N, P444D, R445H, Y448K, P456N, I462M, S463P, M466D, I467E, and P473L;
i) L16M, R26G, G30R, V41A, E42N, A64K, L76M, N102Y, I105L, D111E, T127R, I130F, S147T, Y155F, T157H, L167F, V171L, V177I, S189F, I205Q, A213K, F215W, K216M, V222A, N231D, R242S, L253Q, N266G, K270Q, K281S, Y286C, I292V, K302G, R328A, K331A, R334H, A335C, S336V, S351P, Q352E, M354L, S355C, Q362R, L365M, G368A, P386R, T391Q, R394L, H409L, L411M, N421D, Q422E, E425H, Q427S, D437N, H440S, L443N, P444D, R445H, Y448K, P456N, I462M, S463P, and P473L;
j) R26G, G30R, V41A, A64K, L76M, N102Y, I105L, D111E, T127R, I130F, S147T, Y155F, T157R, L167F, V171L, V177I, S189F, I205Q, A213K, F215W, K216M, V222A, N231D, R242S, L253Q, N266G, K270Q, D277Q, K281S, Y286C, I292A, K302G, R328A, K331A, R334N, A335C, S336T, S351P, Q352E, M354L, S355C, Q362R, L365M, G368A, P386S, T391Q, R394L, H409L, L411M, N421D, Q422E, E425H, Q427S, D437N, H440S, L443N, P444D, R445H, Y448K, P456N, I462M, S463P, and P473L;
k) Y9H, R26G, G30R, V41K, A64W, N102F, I105L, T127R, I130F, S147T, T157H, L167F, V171L, V177I, I205Q, F215W, N231D, L253Q, N266G, K270Q, D277Q, Y286C, I292A, K302G, R328A, K331A, R334H, Q352E, M354L, S355C, G368A, P386S, T391Q, R394L, L411M, N421D, D437N, P444D, R445H, S463P, and P473L;
l) Y9H, R26G, G30R, V41R, A64W, N102Y, I105L, T127R, I130F, S147T, T157H, V171L, V177I, I205Q, F215W, V222A, N231D, R242S, L253Q, N266G, K270Q, Y286C, I292A, K302G, R328A, K331A, R334H, A335C, S336V, Q352E, M354L, S355C, G368A, N374Q, P386S, T391Q, R394L, L411M, N421D, E425H, D437N, H440S, P444D, R445H, P456N, S463P, and P473L;
m) Y9H, R26G, G30R, V41K, A64W, N102Y, T127R, I130F, S147T, T157H, V171L, I205Q, N231D, L253Q, N266G, K270Q, D277Q, Y286C, I292A, K302G, R328A, K331A, R334H, M354L, G368A, P386S, T391Q, R394L, L411M, N421D, E425H, D437N, P444D, S463P, and P473L; or
n) Y9H, R26G, G30R, V41A, A64W, N102Y, I105L, T127R, I130F, S147T, T157H, L167F, V171L, V177I, I205Q, F215W, V222A, N231D, R242S, L253Q, N266G, K270Q, D277Q, Y286C, I292A, K302G, R328A, K331A, R334H, A335C, Q352E, M354L, S355C, G368A, N374Q, P386S, T391Q, R394L, L411M, N421D, E425H, D437N, H440S, P444D, R445H, P456N, S463P, and P473L.

The present invention also provides a beta amylase variant of a wild-type beta amylase from sweet potato of SEQ ID NO: 4 comprising one or more mutations selected from I4T, V7S, G23D, A27R, V30N, V37C, D39A, D39Q, D39W, K42E, K42H, K42M, K42Q, V44L, K45A, G47A, G48N, G47N, C49E, C49V, M53V, I60N, I60V, I61V, A63K, A63S, K67M, K67Q, K67R, A73G, A73Y, R75H, R75K, L80M, V81C, K82R, K87H, I88L, A90V, V105C, V105F, V105H, F106S, F106T, I113V, Q115E, N127D, A129S, L137I, V141C, L146V, E161Q, E161R, D165N, A173D, D175L, E196Q, T197N, V201A, V280I, V201S, M215L, V216K, V216R, V216Q, E221N, V223A, Q225K, M233L M233S, G235K, E249T, R252K, D260E, T267Q, N271Q, K272C, K272S, K272V, I274E, I275Q, I275H, G277L, Q279R, E282A, V287I, L291C, L291T, R292G, R292K, N294S, N294A, N294T, I295L, A296S, K298R, S300A, N307K, H308T, V309R, F319Y, V322T, A323R, G324N, P330A, A332L, T341I, E357N, Q363E, E364A, E364G, E364Q, S371N, S372A, G373A, K375D, Y377D, Y377G, D379H, T392A, T392Q, L398M, L408K, L408R, N409E, Q430E, T431E, T431N, D432N D432Q, D432S F434Y, L436I, K439A, K439N, K442D, P450H, S451C, S451N, N453E, A454V, S456D, A458P, N464K, and D486N.

The present invention also provides a beta amylase variant of a wild-type beta amylase from sweet potato of SEQ ID NO: 4 comprising mutations:
a) I4T, V7S, D39A, K42H, V44L, G47A, G48N, C49V, I60V, A63K, K67M, A73Y, R75H, K82R, K87H, I88L, A90V, V105H, F106T, A129S, L137I, V141C, L146V, E161R, A173D, D175L, T197N, V201A, V216Q, E249T, K272S, I274E, E282A, L291C, R292G, N294S, A296S, K298R, V309R, F319Y, V322T, G324N, P330A, A332L, T341I, E357N, Q363E, E364Q, S371N, S372A, G373A, Y377D, D379H, T392A, L408R, K439A, S451C, A454V, and D486N;
b) I4T, V7S, K42H, V44L, K45A, G47A, G48N, C49V, I60V, A63K, K67Q, R75H, K87H, I88L, A90V, V105F, F106T, A129S, L137I, E161R, A173D, D175L, T197N, V201A, V216R, E249T, K272S, E282A, V287I, L291T, N294S, A296S, K298R, V309R, V322T, G324N, P330A, T341I, E357N, Q363E, E364Q, S371N, G373A, Y377G, T392A, L408R, T431E, K439A, S451N, A454V, and D486N;
c) D486N;
d) I4T, V7S, G23D, V30N, D39A, K42M, V44L, K45A, G47A, G48N, C49V, I60N, K67M, A73G, R75H, L80M, K87H, I88L, A90V, F106T, I113V, Q115E, A129S, L137I, V141C, L146V, E161Q, A173D, D175L, E196Q, T197N, V201A, M215L, V216K, E221N, V223A, Q225K, M233S, G235K, E249T, N271Q, K272C, I274E, I275Q, G277L, E282A, L291C, R292G, N294T, I295L, A296S, K298R, S300A, N307K, V309R, F319Y, V322T, G324N, P330A, E357N, Q363E, E364G, S371N, S372A, G373A, Y377G, D379H, T392A, L398M, L408R, N409E, T431N, D432Q, K439A, P450H, S451C, N453E, A454V, S456D, and D486N;
e) I4T, V7S, D39A, K42Q, V44L, K45A, G47A, G48N, C49V, I60V, K67Q, A73Y, R75H, K87H, I88L, A90V, V105F, F106T, A129S, L137I, V141C, E161R, A173D, D175L, T197N, V201A, V216Q, E249T, K272S, I274E, E282A, L291C, R292G, N294T, A296S, K298R, V309R, F319Y, V322T, G324N, P330A, E357N, Q363E, E364A, S371N, S372A, G373A, Y377G, D379H, T392A, L408R, T431E, K439A, S451N, A454V, and D486N;
f) I4T, V7S, G23D, V30N, D39W, K42M, V44L, G47A, G48N, C49E, I60V, A63K, K67Q, A73G, R75H, K87H, I88L, A90V, V105H, F106T, I113V, Q115E, A129S, V141C, L146V, E161R, A173D, D175L, T197N, V201A, V216Q, E221N, G235K, E249T, T267Q, K272S, I274E, I275H, Q279R, E282A, L291C, R292G, N294T, A296S, K298R, V309R, F319Y, V322T, G324N, P330A, E357N, Q363E, E364Q, S371N, S372A, G373A, Y377G, D379H, T392A, L408R, N409E, T431E, D432S, K439A, P450H, S451C, N453E, A454V, S456D, and D486N;
g) I4T, V7S, K42H, V44L, K45A, G47A, G48N, C49V, I60V, K67Q, K87H, I88L, A90V, V105F, A129S, L137I, L146V, E161R, A173D, D175L, T197N, V201A, V216R, E249T, K272C, I275Q, E282A, L291T, N294T, I295L, A296S, K298R, V309R, V322T, G324N, P330A, T341I, E357N, Q363E, E364Q, S371N, G373A, Y377D, T392Q, L408R, T431E, K439A, S451C, A454V, A458P, and D486N;
h) I4T, V7S, K42H, V44L, K45A, G47A, G48N, C49V, I60V, A63K, K67Q, K87H, I88L, A90V, V105C, A129S, L137I, L146V, E161R, A173D, D175L, T197N, V201A, V216R, E249T, K272S, E282A, L291T, N294S, A296S, K298R, V309R, V322T, G324N, P330A, T341I, E357N, Q363E, E364Q, S371N, G373A, Y377G, T392Q, L408K, T431E, K439A, S451N, A454V, and D486N;
i) I4T, V7S, G23D, A27R, V30N, V37C, D39Q, K42M, V44L, K45A, G47A, G48N, C49E, I60N, I61V, A63S, K67Q, A73G, R75H, L80M, V81C, K87H, I88L, A90V, V105H, F106T, I113V, Q115E, N127D, A129S, V141C, L146V, E161R, A173D, D175L, E196Q, T197N, V201A, M215L, V216K, E221N, V223A, Q225K, M233L, G235K, E249T, R252K, N271Q, K272S, I274E, I275Q, G277L, V280I, E282A, L291C, R292G, N294T, I295L, A296S, K298R, S300A, N307K, H308T, V309R, F319Y, V322T, A323R, G324N, P330A, A332L, E357N, Q363E, E364Q, S371N, S372A, G373A, K375D, Y377G, D379H, T392A, L408R, N409E, Q430E, T431N, D432N, K439A, K442D, P450H, S451C, N453E, A454V, S456D, N464K, and D486N;
j) I4T, V7S, K42E, V44L, G47A, G48N, C49V, M53V, I60V, K67Q, R75K, K87H, I88L, A90V, V105F, F106T, A129S, L137I, E161R, D175L, T197N, V201A, V216R, E249T, D260E, T267Q, K272S, E282A, L291T, N294T, A296S, K298R, V309R, V322T, G324N, P330A, E357N, Q363E, E364Q, S371N, G373A, Y377G, T392A, L408R, T431E, K439A, S451C, A454V, and D486N;
k) I4T, V7S, G23D, V30N, D39W, K42M, V44L, K45A, G47A, G48N, C49E, I60N, A63K, K67Q, A73Y, R75H, L80M, K87H, I88L, A90V, F106T, I113V, Q115E, A129S, L137I, V141C, L146V, E161Q, A173D, D175L, E196Q, T197N, V201A, M215L, V216K, E221N, V223A, Q225K, M233S, G235K, E249T, K272V, I274E, G277L, E282A, L291C, R292G, N294T, A296S, K298R, S300A, N307K, V309R, F319Y, V322T, G324N, P330A, E357N, Q363E, E364G, S371N, S372A, G373A, Y377G, D379H, T392A, L398M, L408K, N409E, T431N, D432Q, F434Y, K439A, P450H, S451C, N453E, A454V, S456D, and D486N;
l) I4T, V7S, V30N, D39A, K42Q, V44L, G47A, G48N, C49V, I60V, A63K, K67Q, A73Y, I88L, A90V, V105F, F106T, I113V, A129S, L137I, V141C, L146V, D165N, A173D, D175L, T197N, V201S, V216K, E221N, E249T, K272S, I274E, E282A, L291C, R292G, N294T, A296S, K298R, V309R, F319Y, V322T, G324N, P330A, E357N, Q363E, E364Q, S371N, S372A, G373A, Y377G, D379H, T392A, L408R, T431E, F434Y, K439A, P450H, S451C, A454V, S456D, and D486N;
m) I4T, V7S, V30N, D39W, K42M, V44L, G47A, G48N, C49V, I60V, K67Q, A73Y, R75H, I88L, A90V, V105C, F106T, I113V, A129S, V141C, L146V, E161R, A173D, D175L, T197N, V201A, V216K, E221N, E249T, K272S, I274E, E282A, L291C, R292G, N294S, A296S, K298R, V309R, F319Y, V322T, G324N, P330A, T341I, E357N, Q363E, E364Q, S371N, S372A, G373A, Y377D, D379H, T392A, L408R, K439N, P450H, S451C, A454V, S456D, and D486N;
n) I4T, V7S, G23D, A27R, V30N, V37C, D39Q, K42M, V44L, G47A, G48N, C49E, I60N, I61V, A63S, K67Q, A73Y, L80M, V81C, K87H, I88L, A90V, V105H, F106S, I113V, Q115E, N127D, A129S, L137I, V141C, L146V, E161R, D175L, E196Q, T197N, V201A, M215L, V216K, E221N, V223A, Q225K, M233L, G235K, E249T, R252K, N271Q, K272S, I274E, I275Q, G277L, V280I, E282A, L291C, R292G, N294A, I295L, A296S, K298R, S300A, N307K, H308T, V309R, F319Y, V322T, A323R, G324N, P330A, A332L, T341I, E357N, Q363E, E364G, S371N, S372A, G373A, K375D, Y377G, D379H, T392A, L398M, L408R, N409E, Q430E, T431N, D432Q, L436I, K439A, K442D, P450H, S451C, N453E, A454V, S456D, N464K, and D486N; or
o) I4T, V7S, G23D, V30N, D39W, K42Q, V44L, K45A, G47N, G48N, C49E, I60V, K67R, A73Y, K87H, I88L, A90V, V105F, F106T, I113V, Q115E, A129S, L137I, V141C, L146V, E161Q, A173D, D175L, T197N, V201A, V216K, E221N, G235K, E249T, K272S, I274E, E282A, L291C, R292K, N294S, A296S, K298R, V309R, F319Y, V322T, G324N, P330A, T341I, E357N, Q363E, E364A, S371N, S372A, G373A, Y377D, D379H, T392A, L408R, N409E, T431E, D432Q, K439A, P450H, S451C, N453E, A454V, S456D, and D486N.

The present invention provides a cellulase variant of a wild-type cellulase from Trichoderma reesei of SEQ ID NO: 5 comprising one or more mutations selected from L81C, P83Y, D84G, E85N, A86D, T87Y, G89S, K90Q, and N91C.

The present invention provides a cellulase variant of a wild-type cellulase from Trichoderma reesei of SEQ ID NO: 5 comprising mutations L81C, P83Y, D84G, E85N, A86D, T87Y, G89S, K90Q, and N91C.

The present invention also provides a cellulase variant of a wild-type cellulase from Trichoderma reesei of SEQ ID NO: 18 comprising one or more mutations selected from T42C, V48C, T72C, P83C, E85G, G89C, K90R, Y93C, L113M S125N, E139N, K144Q, G162C, S166A, L167F L169F, D173E, N175S, A178K, N179S, Y181L, N187A, D194C, P198C, S210K, S210T, N217C, L222W, A230S, T237C, D242C, S243A, G247C, S253K, S253Q, S283G, T292H, K294N, R296E, Q297R, P316C, A325G, K329S, I341L, I341M, N345D, R356N, A357N, S361C, N366H, A372K, A372Q, G376D, G376N, V380T, Y381F, T393F, and T393S.

The present invention also provides a cellulase variant of a wild-type cellulase from Trichoderma reesei of SEQ ID NO: 18 comprising mutations:
a) T42C, V48C, T72C, P83C, G89C, K90R, Y93C, S125N, K144Q, G162C, L169F, D173E, N175S, A178K, D194C, P198C, S210K, N217C, L222W, T237C, D242C, S243A, G247C, S253K, S283G, T292H, R296E, P316C, A325G, K329S, I341M, R356N, S361C, A372Q, G376D, and T393S;
b) T42C, V48C, T72C, P83C, G89C, Y93C, E139N, G162C, N175S, N179S, N187A, D194C, P198C, N217C, T237C, D242C, G247C, S283G, Q297R, P316C, A325G, I341L, R356N, A357N, S361C, G376N, V380T, and T393S;
c) T42C, V48C, T72C, P83C, G89C, Y93C, E139N, G162C, D173E, N187A, D194C, P198C, N217C, T237C, D242C, G247C, S283G, Q297R, P316C, R356N, S361C, G376N, and T393F;
d) T42C, V48C, T72C, P83C, G89C, Y93C, E139N, G162C, N179S, N187A, D194C, P198C, N217C, L222W, T237C, D242C, G247C, S283G, Q297R, P316C, A325G, I341M, R356N, S361C, G376N, and T393F;
e) Y39S, T42C, V48C, T72C, P83C, E85G, G89C, K90R, Y93C, L113M, S125N, K144Q, G162C, S166A, L167F, L169F, D173E, N175S, A178K, Y181L, D194C, P198C, S210T, N217C, L222W, T237C, D242C, G247C, S253Q, S283G, T292H, P316C, A325G, K329S, I341M, N345D, R356N, A357N, S361C, A372K, G376D, and T393S;
f) T42C, V48C, T72C, P83C, G89C, K90R, Y93C, L113M, S125N, K144Q, G162C, S166A, L167F, L169F, D173E, N175S, A178K, D194C, P198C, S210T, N217C, L222W, T237C, D242C, S243A, G247C, S283G, T292H, P316C, A325G, K329S, I341M, N345D, R356N, A357N, S361C, A372Q, G376D, and T393S;
g) T42C, V48C, T72C, P83C, G89C, Y93C, E139N, G162C, N187A, D194C, P198C, N217C, T237C, D242C, G247C, S283G, Q297R, P316C, R356N, S361C, G376N, and T393F;
h) T42C, V48C, T72C, P83C, E85G, G89C, Y93C, L113M, E139N, G162C, S166A, L167F, L169F, N175S, N179S, Y181L, N187A, D194C, P198C, N217C, T237C, D242C, G247C, S283G, T292H, Q297R, P316C, A325G, K329S, I341M, N345D, R356N, A357N, S361C, A372K, G376N, V380T, Y381F, and T393S;
i) T42C, V48C, T72C, P83C, G89C, K90R, Y93C, S125N, K144Q, G162C, L169F, D173E, N175S, A178K, D194C, P198C, S210K, N217C, L222W, T237C, D242C, S243A, G247C, S283G, T292H, P316C, A325G, K329S, I341M, R356N, A357N, S361C, N366H, A372Q, G376D, and T393S;
j) T42C, V48C, T72C, P83C, E85G, G89C, K90R, Y93C, L113M, S125N, K144Q, G162C, S166A, L167F, L169F, D173E, N175S, A178K, Y181L, D194C, P198C, S210T, N217C, L222W, A230S, T237C, D242C, G247C, S253Q, S283G, T292H, K294N, P316C, A325G, K329S, I341M, N345D, R356N, A357N, S361C, A372K, G376D, and T393S;
k) T42C, V48C, T72C, P83C, G89C, Y93C, E139N, G162C, S166A, D173E, N175S, N179S, N187A, D194C, P198C, N217C, T237C, D242C, G247C, S283G, P316C, A325G, K329S, I341L, N345D, R356N, A357N, S361C, G376N, V380T, and T393S;
l) T42C, V48C, T72C, P83C, G89C, Y93C, E139N, G162C, N179S, N187A, D194C, P198C, N217C, T237C, D242C, G247C, S283G, Q297R, P316C, A325G, I341M, R356N, S361C, G376N, and T393F;
m) T42C, V48C, T72C, P83C, G89C, Y93C, E139N, G162C, N187A, D194C, P198C, N217C, T237C, D242C, G247C, S283G, P316C, R356N, S361C, G376N, and T393F;
n) T42C, V48C, T72C, P83C, G89C, K90R, Y93C, S125N, K144Q, G162C, L169F, D173E, N175S, A178K, D194C, P198C, S210K, N217C, L222W, T237C, D242C, G247C, S253K, S283G, T292H, R296E, P316C, A325G, K329S, I341M, R356N, S361C, N366H, A372Q, G376D, and T393S;
o) T42C, V48C, T72C, P83C, G89C, Y93C, G162C, D194C, P198C, N217C, T237C, D242C, G247C, P316C, and S361C;
p) T42C, V48C, T72C, P83C, G89C, K90R, Y93C, S125N, K144Q, G162C, L169F, D173E, N175S, A178K, D194C, P198C, S210K, N217C, L222W, T237C, D242C, S243A, G247C, S253K, S283G, T292H, K294N, P316C, A325G, K329S, I341M, R356N, A357N, S361C, A372Q, G376D, and T393S;
q) T42C, V48C, T72C, P83C, G89C, Y93C, E139N, G162C, S166A, N175S, N179S, N187A, D194C, P198C, N217C, T237C, D242C, G247C, S283G, Q297R, P316C, A325G, K329S, I341L, N345D, R356N, A357N, S361C, G376N, V380T, and T393S;
r) T42C, V48C, T72C, P83C, G89C, Y93C, E139N, G162C, S166A, N179S, N187A, D194C, P198C, N217C, T237C, D242C, G247C, S283G, Q297R, P316C, A325G, I341M, R356N, A357N, S361C, G376N, and T393S;
s) T42C, V48C, T72C, P83C, G89C, Y93C, E139N, G162C, D173E, N187A, D194C, P198C, N217C, T237C, D242C, G247C, S283G, P316C, R356N, S361C, G376N, and T393F;
t) T42C, V48C, T72C, P83C, G89C, K90R, Y93C, S125N, K144Q, G162C, L169F, D173E, A178K, D194C, P198C, N217C, L222W, T237C, D242C, G247C, S283G, T292H, P316C, K329S, I341M, R356N, S361C, A372Q, G376D, and T393S;
u) T42C, V48C, T72C, P83C, G89C, K90R, Y93C, L113M, S125N, K144Q, G162C, S166A, L167F, L169F, D173E, N175S, A178K, D194C, P198C, S210T, N217C, L222W, T237C, D242C, G247C, S283G, T292H, P316C, A325G, K329S, I341M, N345D, R356N, A357N, S361C, A372Q, G376D, and T393S;
v) T42C, V48C, T72C, P83C, G89C, K90R, Y93C, S125N, K144Q, G162C, L169F, D173E, A178K, D194C, P198C, S210K, N217C, L222W, T237C, D242C, G247C, S283G, T292H, K294N, P316C, K329S, I341M, R356N, S361C, A372Q, G376D, and T393S;
w) T42C, V48C, T72C, P83C, G89C, Y93C, E139N, G162C, S166A, N179S, N187A, D194C, P198C, N217C, T237C, D242C, G247C, S283G, Q297R, P316C, A325G, R356N, A357N, S361C, G376N, and T393S;
x) T42C, V48C, T72C, P83C, G89C, Y93C, E139N, G162C, S166A, D173E, N175S, N179S, N187A, D194C, P198C, N217C, T237C, D242C, G247C, S283G, Q297R, P316C, A325G, R356N, A357N, S361C, G376N, V380T, and T393S;
y) T42C, V48C, T72C, P83C, E85G, G89C, Y93C, L113M, E139N, G162C, S166A, L167F, L169F, D173E, N175S, N179S, Y181L, N187A, D194C, P198C, N217C, T237C, D242C, G247C, S283G, T292H, P316C, A325G, K329S, I341M, N345D, R356N, A357N, S361C, A372K, G376N, V380T, Y381F, and T393S;
z) T42C, V48C, T72C, P83C, G89C, K90R, Y93C, S125N, K144Q, G162C, L169F, D173E, A178K, D194C, P198C, S210K, N217C, L222W, T237C, D242C, G247C, S253K, S283G, T292H, R296E, P316C, K329S, I341M, R356N, S361C, N366H, A372Q, G376D, and T393S;
aa) T42C, V48C, T72C, P83C, G89C, K90R, Y93C, S125N, K144Q, G162C, L169F, D173E, A178K, D194C, P198C, S210K, N217C, L222W, T237C, D242C, S243A, G247C, S253K, S283G, T292H, P316C, K329S, I341M, R356N, S361C, N366H, A372Q, G376D, and T393S; or
bb) T42C, V48C, T72C, P83C, G89C, K90R, Y93C, S125N, K144Q, G162C, S166A, L169F, D173E, N175S, A178K, D194C, P198C, S210K, N217C, L222W, T237C, D242C, G247C, S283G, T292H, P316C, A325G, K329S, I341M, N345D, R356N, A357N, S361C, A372Q, G376D, and T393S.

The present invention provides a cyclodextrin glucanotransferase variant of a wild-type cyclodextrin glucanotransferase from Bacillus macerans of SEQ ID NO: 6 comprising one or more mutations selected from D34N, D34S, K36T, D56N, D64G, R70C, I84V, D86N, K115N, Q134K, D141S, D150N, L152A, L154A, I155K, T156N, S157L D160I, D160V, R161M, V168T, R171A, N175D, G177S, E180D, D186N, S189N, L191M, L201Y, F238L, K239E, L244W, V250F, F255H, Q260H, Y261L, G264P, S268T, G275S, D277S, Y287F, E303R, E316A, E316K, F321I, R322A, D323K, T325H, T325N, K329F, K329S, K329W, Y313L, V334M, L335I, L335R, A336L, A336Q, S337K, S337R, E339A, E339Q, Q341D, D343N, Y344W, I345A, M348L, D358S, D358T, V362Y G364N, S365G, S365N, G366S, A369K, T370Y, E371H, D397G, G412T, V418I, A421K, S428L, T436G, T438W, E439Q, R440V, V442M, N443T, S455G, S467T, A468T, A469S, A469T, P471S, S473T, S477T, N507T, S526T, N531H, I542T, A554K, G568N, K578Q, K578T, I581V, A585T, A586T, T589A, T589Y, K594Q, K594T, T599G, A600Q, A600T, N602S, F604Y, K605E, K605V, S606P, L620V, T630E, T630Q, A638T, A638V, A639S, T643N, P646T, Y654F, I658M, S663N, S663T, K675Q, I681L, I681V, K684R, T690V, A703T, V706T, and D712T.

The present invention provides a cyclodextrin glucanotransferase variant of a wild-type cyclodextrin glucanotransferase from Bacillus macerans of SEQ ID NO: 6 comprising mutations:
a) D34S, D64G, R70C, I84V, D141S, L152A, L154A, I155K, D160I, R171A, G177S, K239E, Q260H, Y261L, D277S, E303R, E316A, D323K, K329S, L335R, A336Q, E339Q, I345A, D358T, S365N, D397G, G412T, A421K, T436G, P471S, N531H, I542T, K578Q, A585T, T589Y, K594T, T599G, K605E, S606P, A638V, T643N, P646T, S663T, V706T, and D712T;
b) D34S, K36T, D64G, R70C, I84V, D86N, K115N, D141S, D150N, L152A, L154A, I155K, T156N, S157L, D160V, R161M, V168T, R171A, G177S, E180D, L191M, F238L, K239E, F255H, Q260H, Y261L, G275S, D277S, E303R, E316A, R322A, D323K, T325N, K329W, V334M, L335R, A336Q, S337K, E339A, Q341D, D343N, Y344W, I345A, M348L, D358S, V362Y, G364N, S365G, G366S, A369K, T370Y, E371H, D397G, G412T, V418I, A421K, T436G, R440V, N443T, S467T, A468T, A469S, P471S, S473T, S477T, S526T, N531H, I542T, A554K, G568N, K578Q, A585T, A586T, T589A, K594T, T599G, A600T, F604Y, K605V, S606P, T630E, A638V, A639S, T643N, P646T, Y654F, I658M, S663N, I681L, T690V, V706T, and D712T;
c) D34S, D64G, R70C, I84V, D141S, L152A, L154A, I155K, D160I, R171A, G177S, F238L, K239E, Q260H, Y261L, D277S, E303R, E316A, D323K, K329S, L335R, A336Q, E339Q, D343N, I345A, D358T, S365N, D397G, G412T, A421K, T436G, P471S, N531H, I542T, K578Q, A585T, T589Y, K594T, T599G, K605E, S606P, A638V, T643N, P646T, S663T, V706T, and D712T;
d) D34S, D64G, R70C, I84V, D141S, L152A, D160I, R161M, G177S, F238L, Q260H, Y261L, D277S, E303R, E316K, D323K, K329F, L335R, A336L, D397G, A421K, T436G, T438W, N443T, P471S, I542T, A585T, K594T, T599G, K605E, T643N, P646T, V706T, and D712T;
e) D34S, D64G, R70C, I84V, D141S, L152A, D160I, R161M, G177S, F238L, Q260H, Y261L, D277S, E303R, E316K, D323K, K329F, L335R, A336Q, D397G, A421K, T436G, T438W, N443T, P471S, I542T, K578T, A585T, K594Q, T599G, N602S, K605E, T643N, P646T, V706T, and D712T;
f) D34S, D64G, R70C, I84V, D141S, L152A, I155K, D160I, R161M, R171A, G177S, F238L, Q260H, Y261L, D277S, E303R, E316A, D323K, K329S, L335R, A336Q, E339Q, I345A, S365N, D397G, A421K, T436G, N443T, P471S, N531H, I542T, A585T, K594T, T599G, K605E, A638V, T643N, P646T, V706T, and D712T;
g) D34N, K36T, D56N, D64G, R70C, I84V, D86N, K115N, Q134K, D141S, D150N, L152A, L154A, I155K, T156N, S157L, D160I, R161M, V168T, R171A, N175D, G177S, E180D, D186N, S189N, L191M, L201Y, F238L, K239E, L244W, V250F, F255H, Q260H, Y261L, G264P, S268T, G275S, D277S, Y287F, E303R, E316A, R322A, D323K, T325N, K329W, V334M, L335R, A336L, S337K, E339A, Q341D, D343N, Y344W, I345A, M348L, D358S, V362Y, G364N, S365G, G366S, A369K, T370Y, E371H, D397G, G412T, V418I, A421K, S428L, T436G, T438W, E439Q, R440V, N443T, S455G, S467T, A468T, A469T, P471S, S473T, S477T, N507T, S526T, N531H, I542T, A554K, G568N, K578Q, I581V, A585T, A586T, T589A, K594T, T599G, A600T, N602S, F604Y, K605V, S606P, L620V, T630Q, A638V, A639S, T643N, P646T, Y654F, S663T, K675Q, I681V, K684R, T690V, A703T, V706T, and D712T;
h) D34S, D64G, R70C, I84V, D141S, L152A, I155K, D160I, R161M, R171A, G177S, F238L, Q260H, Y261L, D277S, E303R, E316A, D323K, K329S, L335R, A336L, E339Q, I345A, S365N, D397G, A421K, T436G, T438W, P471S, N531H, I542T, A585T, K594T, T599G, K605V, A638V, T643N, P646T, V706T, and D712T;
i) D34S, D64G, R70C, I84V, K115N, D141S, L152A, L154A, I155K, D160I, R171A, G177S, E180D, L191M, F238L, K239E, Q260H, Y261L, D277S, E303R, E316A, F321I, D323K, K329S, V334M, L335R, E339Q, I345A, D358T, V362Y, S365G, G366S, T370Y, E371H, D397G, G412T, A421K, T436G, A469T, P471S, S477T, N531H, I542T, K578Q, A585T, T589Y, K594T, T599G, F604Y, K605V, S606P, T630E, A638T, T643N, P646T, S663T, V706T, and D712T;
j) D34S, K36T, D64G, R70C, I84V, D86N, K115N, D141S, D150N, L152A, L154A, I155K, T156N, S157L, D160I, R161M, V168T, R171A, G177S, E180D, L191M, K239E, L244W, F255H, Q260H, Y261L, G275S, D277S, E303R, Y313L, E316A, R322A, D323K, T325N, K329S, V334M, L335I, A336Q, S337R, E339A, Q341D, D343N, Y344W, I345A, M348L, D358S, V362Y, G364N, S365G, G366S, A369K, T370Y, E371H, D397G, G412T, V418I, A421K, T436G, S467T, A468T, A469T, P471S, S473T, S477T, S526T, N531H, I542T, A554K, G568N, K578Q, A585T, A586T, T589A, K594T, T599G, A600Q, F604Y, K605V, S606P, T630E, A638V, A639S, T643N, P646T, Y654F, S663N, I681L, T690V, V706T, and D712T;
k) D34S, K36T, D64G, R70C, I84V, D86N, K115N, D141S, D150N, L152A, L154A, I155K, T156N, S157L, D160I, R161M, V168T, R171A, G177S, E180D, L191M, K239E, F255H, Q260H, Y261L, G275S, D277S, E303R, E316A, R322A, D323K, T325N, K329W, V334M, L335R, A336Q, S337R, E339A, D343N, Y344W, I345A, D358S, V362Y, G364N, S365G, G366S, T370Y, E371H, D397G, G412T, V418I, A421K, T436G, R440V, A468T, A469T, P471S, S473T, S477T, N531H, I542T, A554K, G568N, K578T, A585T, T589A, K594Q, T599G, A600T, N602S, F604Y, K605V, S606P, T630E, A638V, A639S, T643N, P646T, Y654F, S663N, I681L, V706T, and D712T;
l) D34S, D64G, R70C, I84V, K115N, D141S, L152A, L154A, I155K, D160I, R171A, G177S, E180D, L191M, K239E, Q260H, Y261L, D277S, E303R, E316A, F321I, D323K, T325H, K329S, V334M, L335R, A336Q, D343N, I345A, D358T, V362Y, S365G, G366S, T370Y, E371H, D397G, G412T, A421K, T436G, A469T, P471S, S477T, N531H, I542T, K578Q, A585T, T589Y, K594T, T599G, F604Y, K605V, S606P, T630E, A638T, T643N, P646T, S663T, V706T, and D712T;
m) D34S, K36T, D64G, R70C, I84V, D86N, K115N, D141S, D150N, L152A, L154A, I155K, T156N, S157L, D160I, V168T, R171A, G177S, E180D, L191M, F238L, K239E, F255H, Q260H, Y261L, G275S, D277S, E303R, E316A, R322A, D323K, T325N, K329W, V334M, L335R, A336Q, S337R, E339Q, D343N, Y344W, I345A, D358S, V362Y, G364N, S365G, G366S, T370Y, E371H, D397G, G412T, V418I, A421K, T436G, R440V, A468T, A469T, P471S, S473T, S477T, N531H, I542T, A554K, G568N, K578Q, A585T, T589A, K594T, T599G, A600T, F604Y, K605V, S606P, T630E, A638V, A639S, T643N, P646T, Y654F, S663N, V706T, and D712T; or
n) D34N, K36T, D56N, D64G, R70C, I84V, D86N, K115N, Q134K, D141S, D150N, L152A, L154A, I155K, T156N, S157L, D160I, R161M, V168T, R171A, N175D, G177S, E180D, D186N, S189N, L191M, L201Y, K239E, V250F, F255H, Q260H, Y261L, G264P, S268T, G275S, D277S, Y287F, E303R, E316A, R322A, D323K, T325N, K329S, V334M, L335R, A336L, S337R, E339Q, Q341D, Y344W, I345A, M348L, D358S, V362Y, G364N, S365G, G366S, A369K, T370Y, E371H, D397G, G412T, V418I, A421K, S428L, T436G, T438W, E439Q, V442M, S455G, S467T, A468T, A469T, P471S, S473T, S477T, N507T, S526T, N531H, I542T, A554K, G568N, K578Q, I581V, A585T, A586T, T589A, K594T, T599G, A600T, F604Y, K605V, S606P, L620V, T630Q, A638V, A639S, T643N, P646T, Y654F, S663T, K675Q, I681V, K684R, T690V, A703T, V706T, and D712T.

The present invention provides a cyclodextrin glucanotransferase variant of a wild-type cyclodextrin glucanotransferase from Geobacillus stearothermophilus of SEQ ID NO: 7 comprising one or more mutations selected from I44V, S65A, T72C, G79C, E96D, A113S, D119S, T122A, T135P, I161A, I161C, I161M, D186N, I191L, T198A, S200G, S200M, N212S, N212T, F222Y, F227L, Q232N, Q232P, F233T, F233V, K236R, K239I, K239L, I242A, I242V, L244M, L244W, L246I, M255H, K259R, F263H, F263Q, S268E, S268K, F269L, M270Q, D271H, D271N, V273I, Y274D, D275N, P278G, P278N, S288G, E289G, S294A, N295D, H297Y, H297Y, F298Y, E302N, K315A, L316I, L316V, Q318E, V319A, V319S, N322D, S324N, D326N, D326T, N331H, N331Y, T337Q, A338Q, A340D, D342N, E343W, I345L, M360H, M360L, A361I, A361S, D362L, E363G, E363N, P366N, K368N, K368R, V369L, I371M, L373M, V375F, V382T, N396G, M404D, T406S, N411T, R413T, Q419R, S423A, S427N, S432A, D435S, T436Q, R439L, S443G, I447V, E449K, L465S, K467S, S468N, S470T, S480A, T484E, T484K, Q486V, A489G, G499N, N501D, A503N, A503N, A503S, N508D, G510Q, G515A, G515S, T518A, A522T, E523T, V525H, V525T, I531V, M534S, M534T, H540N, K541Q, K541Q, L542V, T551D, V553K, V553T, N560T, V562A, V562Q, S564N, Q572E, T574V, T576A, T576S, I580V, P581S, A582P, Q590V, T591S, T591V, G593S, Q595G, V596E, V596K, V596T, A598N, A599N, D601V, N621T, W624L, W624S, E626Q, L630I, H635Y, N641D, S643D, L649M, I654V, V662I, V662Y, E668A, G669D, K670T, K675M, K678I, K679R, G681D, G681S, S682Q, I686T, I687T, I687T, S690N, S700N, S701N, T704K, T705K, T705K, V705I, N706I, V706I, N707I, N707I, N708D, and N709D.

The present invention provides a cyclodextrin glucanotransferase variant of a wild-type cyclodextrin glucanotransferase from Geobacillus stearothermophilus of SEQ ID NO: 7 comprising mutations:
a) I44V, T72C, G79C, I161M, D186N, N212T, F222Y, Q232N, F233T, K239L, I242A, M255H, F263H, M270Q, D271H, D275N, P278N, H297Y, E302N, N322D, D326N, N331H, A340D, E343W, M360H, A361S, D362L, E363N, P366N, K368R, V369L, V375F, V382T, N396G, M404D, R413T, Q419R, S423A, D435S, S470T, T484K, Q486V, A489G, G499N, A503N, G510Q, G515S, A522T, V525H, M534T, K541Q, V553K, V562Q, Q590V, Q595G, V596E, A598N, W624S, L649M, V662Y, G669D, K670T, K678I, K679R, G681D, I687T, S701N, T705K, V706I, N707I, and N709D;
b) T72C, G79C, I161A, D186N, N212T, F233T, F263Q, M270Q, D271H, D275N, P278N, H297Y, E302N, D326T, A340D, E343W, M360L, L373M, V375F, V382T, M404D, R413T, D435S, S470T, T484E, A503N, G510Q, A522T, V525H, K541Q, Q590V, Q595G, A598N, L649M, G681D, I687T, S701N, T705K, V706I, N707I, and N709D;
c) T72C, G79C, I161A, D186N, N212T, F233T, I242A, F263Q, S268E, M270Q, D271N, D275N, P278G, H297Y, E302N, D326T, A340D, E343W, M360L, P366N, K368R, V369L, V375F, V382T, M404D, R413T, Q419R, S423A, D435S, E449K, S470T, A489G, G499N, A503N, G510Q, G515S, A522T, V525H, K541Q, V553K, V562Q, Q590V, Q595G, A598N, L649M, K678I, G681D, I687T, S701N, T705K, V706I, N707I, and N709D;
d) T72C, G79C, I161A, D186N, F233T, F263Q, M270Q, D271N, D275N, P278N, H297Y, E302N, D326T, A340D, E343W, M360L, V375F, V382T, M404D, R413T, D435S, S470T, T484E, A503N, G510Q, A522T, V525H, K541Q, Q590V, A598N, L649M, G681D, I687T, S701N, T705K, V706I, N707I, and N709D;
e) T72C, G79C, I161A, D186N, N212T, F222Y, Q232N, F233T, K239L, I242A, F263H, S268E, M270Q, D271N, D275N, P278N, H297Y, E302N, D326N, A340D, E343W, M360H, E363N, P366N, K368R, V369L, V375F, V382T, M404D, R413T, Q419R, S423A, D435S, S470T, Q486V, A489G, G499N, A503N, G510Q, G515S, A522T, V525H, M534T, K541Q, V553K, V562Q, Q590V, Q595G, V596E, A598N, L649M, V662Y, K678I, K679R, G681D, I687T, S701N, T705K, V706I, N707I, and N709D;
f) I44V, T72C, G79C, I161M, D186N, S200G, N212T, F222Y, Q232N, F233T, K239I, I242A, M255H, F263Q, S268K, M270Q, D271N, D275N, P278N, H297Y, E302N, L316V, V319A, N322D, D326T, N331H, A340D, D342N, E343W, M360L, A361S, D362L, E363N, P366N, K368R, V369L, V375F, V382T, N396G, M404D, R413T, Q419R, S423A, D435S, T436Q, L465S, S470T, T484E, A489G, G499N, A503N, G510Q, G515S, A522T, V525H, M534S, K541Q, V553K, V562Q, Q590V, Q595G, V596E, A598N, W624S, S643D, L649M, V662Y, E668A, K670T, K678I, G681D, I687T, S701N, T705K, N707I, and N709D;
g) I44V, S65A, E96D, A113S, D119S, T122A, T135P, I191L, T198A, S200M, N212S, F227L, Q232P, F233V, K236R, I242V, L244M, L246I, F269L, V273I, Y274D, D275N, S294A, F298Y, D326N, I345L, A361I, E363G, I371M, T406S, S427N, S432A, S443G, I447V, L465S, K467S, S468N, S480A, A489G, A503S, N508D, T518A, A522T, V525T, I531V, K541Q, L542V, V553T, N560T, V562A, S564N, T574V, T576A, I580V, P581S, A582P, T591S, G593S, V596T, N621T, W624L, E626Q, L630I, H635Y, N641D, L649M, I654V, V662I, G681S, S682Q, I686T, S700N, T704K, V705I, N706I, and N708D;
h) I44V, T72C, G79C, I161M, D186N, S200G, N212T, F222Y, Q232N, F233T, K239I, I242A, L244W, M255H, F263H, D271H, D275N, P278N, S288G, E289G, N295D, H297Y, E302N, K315A, Q318E, V319S, N322D, S324N, D326T, N331H, T337Q, A338Q, A340D, D342N, E343W, M360L, A361S, D362L, E363N, P366N, K368R, V369L, V375F, V382T, N396G, M404D, R413T, Q419R, S423A, D435S, T436Q, I447V, L465S, S470T, T484K, A489G, G499N, N501D, A503N, G510Q, G515A, A522T, E523T, V525H, M534S, H540N, K541Q, T551D, V553K, V562Q, Q572E, T576S, Q590V, T591V, Q595G, V596K, A598N, A599N, D601V, W624S, S643D, L649M, V662Y, E668A, K670T, K675M, K678I, K679R, G681D, I687T, S690N, S701N, T705K, N707I, and N709D;
i) T72C, G79C, I161A, D186N, N212T, F233T, F263Q, S268E, D271N, D275N, P278N, H297Y, E302N, D326T, N331Y, A340D, E343W, M360L, P366N, K368R, V369L, V375F, V382T, M404D, R413T, Q419R, S423A, D435S, E449K, S470T, T484E, A489G, G499N, A503N, G510Q, G515S, A522T, V525H, K541Q, V553K, V562Q, Q590V, Q595G, A598N, L649M, K678I, G681D, I687T, S701N, T705K, V706I, N707I, and N709D;
j) T72C, G79C, I161C, D186N, N212T, F222Y, Q232N, F233T, K239L, I242A, F263H, S268E, M270Q, D271N, D275N, P278N, H297Y, E302N, N322D, D326N, A340D, E343W, M360L, E363N, P366N, K368R, V369L, V375F, V382T, M404D, R413T, Q419R, S423A, D435S, S470T, T484K, A489G, G499N, A503N, G510Q, G515S, A522T, V525H, M534T, K541Q, V553K, V562Q, Q590V, Q595G, V596E, A598N, L649M, V662Y, K678I, K679R, G681D, I687T, S701N, T705K, V706I, N707I, and N709D;
k) T72C, G79C, I161C, D186N, F233T, I242A, F263Q, M270Q, D271N, D275N, P278N, H297Y, E302N, D326T, A340D, E343W, M360L, L373M, V375F, V382T, M404D, R413T, D435S, S470T, T484E, A503N, G510Q, A522T, V525H, K541Q, Q590V, A598N, L649M, G681D, I687T, S701N, T705K, V706I, N707I, and N709D;
l) T72C, G79C, I161A, D186N, N212T, F233T, I242A, F263Q, M270Q, D271H, D275N, P278N, H297Y, E302N, D326T, A340D, E343W, M360L, L373M, V375F, V382T, M404D, R413T, D435S, S470T, T484E, A503N, G510Q, A522T, V525H, K541Q, Q590V, Q595G, A598N, L649M, G681D, I687T, S701N, T705K, V706I, N707I, and N709D;
m) I44V, T72C, G79C, I161M, D186N, S200G, N212T, F222Y, Q232N, F233T, K239I, I242A, M255H, F263Q, S268K, D271N, D275N, P278N, H297Y, E302N, L316I, V319A, D326T, N331H, A340D, D342N, E343W, M360H, A361S, D362L, E363N, P366N, K368R, V369L, V375F, N396G, M404D, R413T, Q419R, S423A, D435S, T436Q, L465S, S470T, T484E, A489G, G499N, A503N, G510Q, G515S, A522T, V525H, M534S, K541Q, V553K, V562Q, Q590V, Q595G, V596K, A598N, W624S, S643D, L649M, V662Y, E668A, G669D, K670T, K678I, K679R, G681D, I687T, S701N, T705K, N707I, and N709D;
n) I44V, T72C, G79C, I161M, D186N, N212T, F222Y, Q232N, F233T, K239L, I242A, M255H, K259R, F263H, S268K, M270Q, D271H, D275N, P278G, H297Y, E302N, L316V, D326N, N331H, A340D, E343W, M360H, A361S, D362L, E363N, P366N, K368N, V369L, V375F, V382T, N396G, M404D, R413T, Q419R, S423A, D435S, S470T, T484E, A489G, G499N, A503N, G510Q, G515S, A522T, V525H, M534T, K541Q, V553K, V562Q, Q590V, Q595G, V596E, A598N, W624S, L649M, V662Y, G669D, K670T, K678I, K679R, G681D, I687T, S701N, T705K, V706I, N707I, and N709D;
o) T72C, G79C, G681D, I687T, S701N, T705K, V706I, N707I, and N709D; or
p) I44V, T72C, G79C, E96D, I161M, D186N, S200G, N212T, F222Y, Q232N, F233T, K239L, I242A, L244W, M255H, F263H, S268K, M270Q, D271N, D275N, P278N, S288G, E289G, N295D, H297Y, E302N, K315A, Q318E, V319A, N322D, S324N, D326T, N331H, T337Q, A340D, D342N, E343W, M360L, A361S, D362L, E363N, P366N, K368R, V369L, V375F, V382T, N396G, M404D, N411T, R413T, Q419R, S423A, D435S, T436Q, R439L, I447V, L465S, S470T, T484E, A489G, G499N, N501D, A503N, G510Q, G515A, E523T, V525H, M534S, H540N, K541Q, T551D, V553K, V562Q, Q572E, T576S, Q590V, T591V, Q595G, V596K, A598N, A599N, D601V, W624S, S643D, L649M, V662Y, E668A, G669D, K670T, K675M, K678I, K679R, G681D, I687T, S690N, S701N, T705K, N707I, and N709D.

The present invention also provides a glutaminase variant of a wild-type glutaminase from Bacillus amyloliquefaciens of SEQ ID NO: 8 comprising one or more mutations selected from L14M, A15Q, Q19H, V22I, H24E, H24G, H24Y, P27K, F28Y, S29T, S29A, V30S, V30A, V30K, N31Q, N31E, K33Q, S34C, S34V, N36D, G42A, E43K, I44V, D46K, S47N, I53M, I53V, I53M, L54C, E55T, E55Y, P56T, P56K, S59T, M60C, H62G, E65D, P69S, L72M, S80T, A83L, S87D, R88N, V99M, R111N, I114M, H115N, K116N, G118A, F121C, I130L, V132I, L135M, L135I, P137R, P137Q, T140S, H142E, H142Y, N143E, E146G, E146N, Y147R, Y147F, F149Y, V151F, F152M, E153R, K154T, I156A, K158R, P160L, H161T, H161R, H163N, F167Y, Q172E, T173H, E186S, F189N, F189I, F189M, F189M, D193E, E199D, L202F, K203Q, S209V, T211C, L216R, L216K, I217M, L219A, I220Y, H223N, R231G, R231Q, R231H, K241Q, K244L, L248M, V265L, S282H, E286Q, E286A, Q287A, Q287S, H290Q, N291S, H317Q, I318M, E323D and E323G.

The present invention also provides a glutaminase variant of a wild-type glutaminase from Bacillus amyloliquefaciens of SEQ ID NO: 8 comprising mutations:
a) L14M, A15Q, V22I, H24E, P27K, F28Y, S29T, V30S, N31Q, S34C, N36D, G42A, E43K, I44V, D46K, S47N, I53M, L54C, E55T, P56T, M60C, S80T, A83L, S87D, R88N, V99M, R111N, I114M, K116N, G118A, L135M, H142E, N143E, E146G, Y147R, V151F, E153R, I156A, K158R, P160L, H161T, H163N, F167Y, T173H, E186S, F189N, E199D, L202F, K203Q, S209V, T211C, L216R, I217M, L219A, I220Y, H223N, R231G, K241Q, K244L, L248M, V265L, S282H, E286Q, Q287A, H317Q, I318M, and E323G;
b) A15Q, P27K, S34C, G42A, D46K, S47N, E55T, P56T, A83L, R111N, K116N, N143E, Y147R, V151F, H163N, F189I, S209V, T211C, L216R, L219A, H223N, R231Q, K241Q, K244L, L248M, S282H, E286Q, Q287A, and I318M;
c) I318M;
d) H24G;
e) Q19H, H24Y, S29A, V30A, K33Q, I53V, S59T, E65D, P69S, L72M, H115N, I130L, V132I, L135I, T140S, H142Y, N143E, Y147F, F149Y, F152M, K154T, H161R, Q172E, D193E, R231H, H290Q, N291S, and I318M;
f) A15Q, H24E, P27K, F28Y, S29A, N31Q, S34C, G42A, E43K, D46K, S47N, L54C, E55T, P56T, M60C, H62G, S80T, A83L, R111N, K116N, F121C, L135M, P137R, N143E, E146G, Y147R, V151F, P160L, H161T, H163N, E186S, F189M, L202F, K203Q, S209V, T211C, L216R, I217M, L219A, H223N, R231G, K241Q, K244L, L248M, V265L, S282H, E286Q, Q287A, I318M, and E323D;
g) A15Q, P27K, S34C, G42A, D46K, S47N, E55T, P56T, H62G, A83L, R111N, K116N, P137R, N143E, Y147R, V151F, H163N, F189M, S209V, T211C, L216R, L219A, H223N, R231G, K241Q, K244L, L248M, S282H, E286Q, Q287A, and I318M;
h) P27K, S34C, D46K, S47N, E55Y, H62G, A83L, R111N, K116N, P137R, N143E, Y147R, V151F, H163N, F189M, S209V, T211C, L216K, L219A, R231Q, K241Q, L248M, S282H, E286Q, Q287A, and I318M;
i) A15Q, H24E, P27K, F28Y, S29A, N31Q, S34C, G42A, E43K, D46K, S47N, L54C, E55T, P56T, M60C, H62G, S80T, A83L, R111N, K116N, L135M, P137R, N143E, E146N, Y147R, V151F, P160L, H161T, H163N, E186S, F189M, L202F, K203Q, S209V, T211C, L216R, I217M, L219A, H223N, R231G, K241Q, K244L, L248M, V265L, S282H, E286Q, Q287A, I318M, and E323D; and
j) L14M, A15Q, H24E, P27K, F28Y, S29A, V30S, N31Q, S34C, N36D, G42A, E43K, D46K, S47N, I53M, L54C, E55T, P56T, M60C, S80T, A83L, R88N, V99M, R111N, I114M, K116N, G118A, L135M, H142E, N143E, E146G, Y147R, V151F, K158R, P160L, H161T, H163N, F167Y, T173H, E186S, F189I, L202F, K203Q, S209V, T211C, L216R, I217M, L219A, I220Y, H223N, R231G, K241Q, K244L, L248M, V265L, S282H, E286A, Q287A, H317Q, I318M, and E323D;
k) A15Q, P27K, F28Y, S29T, S34C, G42A, D46K, S47N, E55T, P56K, M60C, H62G, A83L, R111N, K116N, P137Q, N143E, Y147R, V151F, H161T, H163N, F189M, L202F, S209V, T211C, L216R, I217M, L219A, H223N, R231Q, K241Q, K244L, L248M, V265L, S282H, E286Q, Q287A, I318M, and E323D;
l) A15Q, P27K, F28Y, S29T, S34C, G42A, D46K, S47N, E55T, P56K, M60C, A83L, R111N, K116N, P137Q, N143E, Y147R, V151F, P160L, H161T, H163N, F189I, L202F, S209V, T211C, L216R, I217M, L219A, H223N, R231G, K241Q, K244L, L248M, V265L, S282H, E286Q, Q287A, I318M, and E323D;
m) A15Q, P27K, S29T, S34C, G42A, D46K, S47N, E55T, P56T, M60C, H62G, A83L, R111N, K116N, P137R, N143E, Y147R, V151F, H163N, F189M, L202F, S209V, T211C, L216R, L219A, H223N, R231G, K241Q, K244L, L248M, S282H, E286Q, Q287S, I318M, and E323D;
n) P27K, S34C, D46K, S47N, E55Y, H62G, A83L, R111N, K116N, N143E, Y147R, V151F, H163N, F189M, S209V, T211C, L216K, L219A, R231Q, K241Q, L248M, S282H, E286Q, Q287A, and I318M;
o) L14M, A15Q, H24E, P27K, F28Y, S29T, V30S, N31Q, S34V, N36D, G42A, E43K, D46K, S47N, I53M, L54C, E55T, P56T, M60C, H62G, S80T, A83L, R88N, V99M, R111N, K116N, L135M, P137R, H142E, N143E, E146G, Y147R, V151F, K158R, P160L, H161T, H163N, F167Y, T173H, E186S, F189M, L202F, K203Q, S209V, T211C, L216R, I217M, L219A, I220Y, H223N, R231G, K241Q, K244L, L248M, V265L, S282H, E286A, Q287S, H317Q, I318M, and E323D;
p) A15Q, P27K, S29A, S34C, G42A, D46K, S47N, E55T, P56T, M60C, H62G, A83L, R111N, K116N, N143E, Y147R, V151F, H163N, F189I, L202F, S209V, T211C, L216R, L219A, H223N, R231G, K241Q, K244L, L248M, S282H, E286Q, Q287A, I318M, and E323D; or
q) L14M, A15Q, V22I, H24E, P27K, F28Y, S29A, V30K, N31E, S34C, N36D, G42A, E43K, I44V, D46K, S47N, I53M, L54C, E55T, P56T, M60C, H62G, S80T, A83L, S87D, R88N, V99M, R111N, I114M, K116N, G118A, L135M, P137Q, H142E, N143E, E146G, Y147R, V151F, E153R, I156A, K158R, P160L, H161T, H163N, F167Y, T173H, E186S, F189N, E199D, L202F, K203Q, S209V, T211C, L216R, I217M, L219A, I220Y, H223N, R231G, K241Q, K244L, L248M, V265L, S282H, E286Q, Q287A, H317Q, I318M, and E323G.

The present invention provides a protease variant of wild-type protease from Bacillus amyloliquefaciens of SEQ ID NO: 9 comprising one or more mutations selected from F339S, F339R, D340E, A347D, A347G, E350P, Q353G, M354I, V355A, L357V, F362H, K364R, Q365P, E375G, D377E, F381Q, T382V, G384E, T385K, R405K, D409K, K410R, K411P, I412L, Y413F, Y413K, S415G, S415Y, S415G.

The present invention provides a protease variant of wild-type protease from Bacillus amyloliquefaciens of SEQ ID NO: 9 comprising mutations:
a) F339S, A347D, E350P, Q353G, M354I, V355A, L357V, F362H, K364R, Q365P, E375G, D377E, F381Q, T382V, G384E, T385K, R405K, D409K, K410R, K411P, I412L, and S415G;
b) F339S, D340E, A347D, E350P, Q353G, M354I, V355A, L357V, F362H, K364R, Q365P, E375G, D377E, F381Q, T382V, G384E, T385K, R405K, D409K, K410R, K411P, I412L, and S415Y;
c) F339S, D340E, A347D, E350P, Q353G, M354I, V355A, L357V, F362H, K364R, Q365P, E375G, D377E, F381Q, T382V, G384E, T385K, R405K, D409K, K410R, K411P, I412L, and S415G;
d) F339S, D340E, A347G, E350P, Q353G, M354I, V355A, L357V, F362H, K364R, Q365P, E375G, D377E, F381Q, T382V, G384E, T385K, R405K, D409K, K410R, K411P, I412L, Y413F, and S415G;
e) F339S, D340E, A347G, E350P, Q353G, M354I, V355A, L357V, F362H, K364R, Q365P, E375G, D377E, F381Q, T382V, G384E, T385K, R405K, D409K, K410R, K411P, I412L, Y413F, and S415Y;
f) F339R, D340E, A347G, E350P, Q353G, M354I, V355A, L357V, F362H, K364R, Q365P, D377E, F381Q, T382V, G384E, T385K, R405K, D409K, K410R, K411P, I412L, Y413F, and S415Y;
g) F339S, A347D, E350P, Q353G, M354I, V355A, L357V, F362H, Q365P, E375G, D377E, F381Q, T382V, G384E, T385K, R405K, D409K, K410R, K411P, I412L, and S415G;
h) F339S, A347D, E350P, Q353G, M354I, V355A, L357V, F362H, K364R, Q365P, E375G, D377E, F381Q, T382V, G384E, T385K, R405K, D409K, K410R, K411P, I412L, Y413K, and S415G;
i) F339S, D340E, A347D, E350P, Q353G, M354I, V355A, L357V, F362H, K364R, Q365P, E375G, D377E, F381Q, T382V, G384E, T385K, R405K, D409K, K410R, K411P, I412L, Y413K, and S415G; or
j) F339S, D340E, A347G, E350P, Q353G, M354I, V355A, L357V, F362H, K364R, Q365P, E375G, D377E, F381Q, T382V, G384E, T385K, R405K, D409K, K410R, K411P, I412L, Y413K, and S415G.

The present invention provides a pullulanase variant of a wild-type pullulanase from Bacillus sp. of SEQ ID NO: 10 comprising one or more mutations selected from V2M, R5K, S7T, V12L, D14K,D14K, M15Y, M15F, M15Y, I17R, I17K, I17K, L21T, I22M, P23S, A24L, I29G, M30K, T31S, R35Y, R35H, L36M, E37R, E37K, T38V, I40K, T41G, D42R, D42T, D42H, D42N, P44T, L45E, L45K, A46T, V47I, R48I, R48V, Y51W, S52E, L53K, L53Y, L53E, L53H, L53N, E54T, A55T, A55D, A55E, Y59L, V60Y, V60F, C61R, C61K, V62I, V62L, R76C, H81N, D97W, D97Y, D97H, D98M, E99K, L146M, Q180P, A204D, H211N, T220M, L222I, I234T, L242A, D246G, A250S, A250N, N251E, N251K, N251E, S253N, S255T, A258D, A258S, V260I, V260M, V260I, L271M, G278S, E282N, K283H, K283N, K283G, L285Q, A287Q, A287T, N293D, H296Q, F297Y, F297W, F298N, A299V, H309A, Q312A, K315V, K315I, K315V, T316V, I322V, I322V, L325F, L325V, Q327E, H328A, L334M, F338Y, K343D, K343I, K343S, P348N, P348S, P348H, T352L, F357Y, F358Y, H360M, H360Y, D361N, E362K, C363D, M365N, M365H, P366L, P366Y, V372C, R381K, A383M, A388V, A388I, C390M, V391C, V391M, V392T, V392K, V392L, V392I, V392C, L395A, L395Q, E396Q, N399H, V400M, V400F, R404K, L408M, I410V, L411H, D414E, V416M, L417N, Y418Q, Y418E, M419I, K420R, K420A, E421R, K422E, A423C, A423L, T424D, T424S, K425S, A426L, A426I, P428K, P428R, G429S, I430F, I430L, L431M, F433Y, L439M, A440N, A440G, P444S, A450N, A450S, A450C, A452Q, A452K, P455S, G461A, G461H, F462H, M466W, M466R, V471I, N474S, H477N, A480D, T481K, L485N, L485T, G488N, G488E, E489H, E489G, S490K, A491E, A491F, Q492E, Q492K, A493D, A493T, V494M, H496A, G497A, I518V, E522S, S523C, M533L, M533L, S534A, F535A, F535I, F535A, L537M, E540D, S543D, S543E, R544E, R544D, K545R, S547A, S547Q, S547A, I556V, L558T, A559S, I565M, I565V, S567A, Q576N, V578N, V578D, V578D, Q583N, D594H, R596K, E597A, E597S, T598K, T598E, F599Y, K600Y, V603F, H604K, S611A, K614R, A615S, P617S, L621M, R622Q, A625E, L631V, C633F, T635Q, T635E, K637N, K637E, K637D, H639D, H639G, H639D, L640V, I641F, I641L, A642G, R644W, L648N, D649G, D649H, D649N, V651Y, E653S, I658V, I658M, V659C, H661C, P665A, D666N, D666H, W670I, N674D, I676G, I676E, P677T, R679H, S685K, S685E, G686K, F687A, E689I, E689T, T692I, E693Q, K695V, T697R, V698K, A699R, A699Q, V700L, N701H, I703G, G704A, T705G, I707W, L708V, L708V, Y709L, Y709M, Y709V, L710T, L710M, A711I, A711V, and A711Y.

The present invention provides a pullulanase variant of a wild-type pullulanase from Bacillus sp. of SEQ ID NO: 10 comprising mutations:
a) M15Y, I17R, L21T, P23S, A24L, I29G, R35Y, L36M, I40K, T41G, D42R, V47I, R48I, L53K, E54T, A55T, Y59L, V60Y, C61R, V62I, H81N, D97W, Q180P, A204D, T220M, I234T, L242A, D246G, A250S, N251E, S253N, A258D, V260I, E282N, L285Q, A287Q, F297Y, F298N, Q312A, K315V, T316V, I322V, L325F, L334M, F338Y, K343D, P348N, T352L, H360M, E362K, C363D, M365N, P366L, V372C, A383M, C390M, L395A, N399H, V400M, L408M, I410V, L411H, Y418Q, M419I, K420R, A423C, T424D, A426L, G429S, A440N, A450N, P455S, M466W, N474S, H477N, A480D, T481K, L485N, G488N, E489H, Q492E, V494M, M533L, F535A, L537M, E540D, S543D, R544E, K545R, S547A, A559S, I565M, S567A, V578N, Q583N, E597A, T598K, V603F, H604K, S611A, P617S, L621M, R622Q, A625E, L631V, C633F, T635Q, K637N, H639D, I641F, L648N, D649G, V651Y, E653S, I658V, V659C, P665A, W670I, N674D, I676G, S685K, G686K, F687A, E689I, T692I, E693Q, V698K, A699R, I703G, T705G, I707W, Y709L, L710T, and A711V;
b) M15F, I17K, L21T, I29G, T31S, R35Y, E37R, I40K, T41G, D42T, A46T, R48V, L53Y, R76C, L222I, L242A, D246G, N251K, S253N, V260M, E282N, A287Q, F297Y, F298N, A299V, Q312A, K315I, I322V, L325F, F357Y, H360Y, C363D, M365N, V372C, A383M, V392T, L395Q, N399H, L408M, I410V, Y418E, K420R, A423C, T424D, A426I, G429S, I430F, L431M, F433Y, F462H, M466R, H477N, A480D, T481K, G488N, Q492K, A493D, F535A, S543D, R544E, A559S, Q576N, V578D, E597S, S611A, C633F, K637N, L648N, D649H, V651Y, I658V, V659C, I676G, G686K, E689I, N701H, Y709M, and A711Y;
c) V2M, R5K, S7T, V12L, D14K, M15Y, I17R, L21T, I22M, P23S, A24L, I29G, M30K, R35Y, L36M, T38V, I40K, T41G, D42R, V47I, R48I, Y51W, S52E, L53K, E54T, A55D, Y59L, V60Y, C61K, V62I, H81N, D97Y, D98M, E99K, Q180P, A204D, T220M, I234T, L242A, D246G, A250S, N251E, S253N, A258D, V260I, L271M, E282N, K283H, L285Q, A287Q, N293D, F297Y, F298N, Q312A, K315V, T316V, I322V, L325F, Q327E, L334M, F338Y, K343D, P348N, T352L, F357Y, H360M, D361N, C363D, M365N, P366L, V372C, R381K, A383M, A388V, V392K, L395A, E396Q, N399H, V400F, R404K, L408M, I410V, L411H, D414E, V416M, L417N, Y418E, M419I, K420A, E421R, K422E, A423L, T424D, K425S, A426L, P428K, G429S, A440G, A450N, A452Q, P455S, G461A, M466W, V471I, N474S, H477N, A480D, T481K, L485N, G488N, E489H, S490K, Q492E, A493D, V494M, S523C, M533L, F535A, L537M, E540D, S543D, R544E, K545R, S547A, I556V, A559S, I565M, S567A, V578D, Q583N, E597A, T598E, F599Y, K600Y, V603F, H604K, S611A, K614R, A615S, P617S, L621M, R622Q, A625E, L631V, C633F, T635Q, K637N, H639G, L640V, I641L, R644W, L648N, D649N, V651Y, E653S, I658V, V659C, H661C, P665A, D666N, W670I, N674D, I676G, S685K, G686K, F687A, E689I, T692I, E693Q, T697R, V698K, A699Q, I703G, G704A, T705G, I707W, L708V, Y709V, L710T, and A711V;
d) V2M, S7T, D14K, M15Y, I17K, L21T, P23S, I29G, M30K, T31S, R35Y, L36M, E37K, I40K, T41G, D42H, V47I, R48V, L53E, E54T, A55D, V60F, C61K, V62L, R76C, H81N, E99K, H211N, L222I, L242A, D246G, A250S, N251K, S253N, A258S, V260M, G278S, E282N, K283N, A287Q, N293D, F297W, F298N, A299V, H309A, Q312A, K315I, I322V, L325F, H328A, K343I, P348S, F357Y, F358Y, H360Y, C363D, M365N, P366L, V372C, A383M, A388V, V391C, V392T, L395A, N399H, V400F, L408M, I410V, V416M, L417N, Y418E, M419I, K422E, A423C, T424D, A426I, G429S, I430F, L431M, F433Y, L439M, A440G, P444S, A450S, F462H, M466R, H477N, A480D, T481K, G488N, E489G, S490K, A491E, Q492E, A493T, H496A I518V, E522S, M533L, S534A, F535A, E540D, S543E, R544E, K545R, I556V, A559S, S567A, Q576N, V578D, D594H, R596K, F599Y, S611A, L631V, C633F, T635Q, K637N, H639G, I641F, L648N, D649N, V651Y, E653S, I658V, V659C, N674D, I676G, P677T, R679H, S685E, G686K, E689I, T692I, E693Q, K695V, T697R, V698K, N701H, I703G, G704A, and L708V;
e) D14K, M15Y, I17K, I29G, T31S, R35Y, E37K, I40K, T41G, D42H, L45E, A46T, R48V, A55E, R76C, H81N, L222I, L242A, D246G, A250S, N251K, S253N, A258S, V260M, G278S, E282N, A287Q, F297Y, F298N, A299V, Q312A, K315I, I322V, L325F, K343I, P348S, F357Y, F358Y, H360Y, C363D, M365N, P366L, V372C, A383M, V391C, V392T, L395A, N399H, L408M, I410V, V416M, Y418E, M419I, K420R, A423C, T424D, A426I, G429S, I430F, L431M, F433Y, L439M, A440N, F462H, M466R, H477N, A480D, T481K, G488N, A491E, Q492K, A493D, F535A, R544E, K545R, A559S, I565V, S567A, Q576N, V578D, D594H, R596K, E597S, S611A, C633F, T635E, K637N, H639D, I641F, L648N, D649H, V651Y, I658V, V659C, I676G, G686K, E689I, T692I, E693Q, V698K, N701H, I703G, Y709M, L710M, and A711Y;
f) M15Y, I17K, L21T, P23S, A24L, I29G, T31S, R35Y, I40K, T41G, R48I, L53Y, V60F, R76C, L222I, L242A, D246G, N251K, S253N, V260M, G278S, E282N, A287Q, F297Y, F298N, A299V, Q312A, K315I, I322V, L325F, F338Y, K343I, F357Y, H360Y, C363D, M365N, V372C, A383M, V391C, V392T, L395Q, N399H, L408M, I410V, Y418E, K420R, A423C, T424D, A426I, G429S, I430F, L431M, F433Y, F462H, M466R, H477N, A480D, T481K, G488N, Q492K, A493D, F535A, S543D, R544E, A559S, Q576N, V578D, S611A, C633F, K637N, L648N, D649H, V651Y, I658V, V659C, I676G, G686K, E689I, N701H, Y709M, and A711Y;
g) R5K, M15Y, I17K, L21T, I22M, P23S, A24L, I29G, R35Y, I40K, T41G, D42R, L45K, R48V, L53H, E54T, A55E, Y59L, V60Y, V62I, D97W, Q180P, T220M, I234T, L242A, D246G, A250S, N251E, S253N, A258D, V260I, E282N, L285Q, A287Q, F297Y, F298N, A299V, Q312A, K315V, T316V, I322V, L325F, L334M, F338Y, K343S, P348N, T352L, E362K, C363D, M365N, P366L, V372C, A383M, A388V, V392L, L395A, N399H, L408M, I410V, V416M, Y418Q, M419I, K420A, A423C, T424D, K425S, A426L, G429S, A450N, P455S, M466W, N474S, H477N, A480D, T481K, L485N, A491F, V494M, F535A, S543E, R544D, I565M, V578N, Q583N, E597A, T598K, V603F, S611A, P617S, R622Q, A625E, L631V, C633F, K637N, H639D, I641F, L648N, D649N, V651Y, E653S, V659C, P665A, D666H, I676G, S685K, G686K, F687A, E689I, T692I, E693Q, V698K, A699R, I703G, Y709V, L710M, and A711V;
h) V2M, S7T, D14K, M15Y, I17K, L21T, I29G, M30K, T31S, R35Y, L36M, E37K, I40K, T41G, D42N, A46T, V47I, R48V, L53H, E54T, A55D, C61K, V62L, R76C, H81N, E99K, H211N, L222I, L242A, D246G, A250S, N251K, S253N, S255T, A258S, V260M, G278S, E282N, K283N, A287Q, N293D, F297W, F298N, A299V, H309A, Q312A, K315I, I322V, L325F, H328A, K343I, P348S, F357Y, F358Y, H360Y, C363D, M365N, P366L, V372C, A383M, A388V, V392T, L395A, N399H, V400F, L408M, I410V, V416M, L417N, Y418E, M419I, K422E, A423C, T424D, A426I, G429S, I430F, L431M, F433Y, L439M, A440G, P444S, A450S, F462H, M466R, H477N, A480D, T481K, G488N, E489G, S490K, A491E, Q492E, A493T, H496A, G497A, I518V, E522S, M533L, S534A, F535A, E540D, S543E, R544E, K545R, I556V, A559S, S567A, Q576N, V578D, D594H, R596K, F599Y, S611A, L631V, C633F, T635Q, K637N, H639D, I641F, L648N, D649N, V651Y, E653S, I658V, V659C, N674D, I676G, P677T, R679H, S685E, G686K, E689I, T692I, E693Q, K695V, T697R, V698K, N701H, I703G, G704A, and L708V;
i) D14K, I17K, L21T, P23S, A24L, I29G, T31S, R35Y, E37K, I40K, T41G, D42H, L45E, R48I, L53Y, A55D, V60F, C61R, R76C, H81N, L222I, L242A, D246G, A250S, N251K, S253N, S255T, A258S, V260M, G278S, E282N, A287Q, F297Y, F298N, A299V, Q312A, K315I, I322V, L325F, K343I, P348S, F357Y, H360Y, C363D, M365N, P366L, V372C, A383M, V391C, V392T, L395Q, N399H, L408M, I410V, Y418E, M419I, K420R, A423C, T424D, A426I, G429S, I430F, L431M, F433Y, L439M, A440N, A450C, F462H, M466R, H477N, A480D, T481K, L485T, G488N, A491E, Q492K, A493D, F535A, S543E, R544E, K545R, A559S, S567A, Q576N, V578D, D594H, R596K, E597S, S611A, C633F, T635E, K637N, H639D, I641F, L648N, D649N, V651Y, I658V, V659C, I676G, G686K, E689I, T692I, E693Q, V698K, N701H, I703G, L710M, and A711I;
j) D14K, M15Y, I17K, L21T, P23S, I29G, T31S, R35Y, E37K, I40K, T41G, D42H, L45E, R48I, L53N, A55D, V60F, C61R, R76C, H81N, L222I, L242A, D246G, A250S, N251K, S253N, A258S, V260M, E282N, K283G, A287Q, F297Y, F298N, A299V, H309A, Q312A, K315I, I322V, L325F, P348S, F357Y, F358Y, H360Y, C363D, M365N, P366L, V372C, A383M, V391M, V392T, L395Q, N399H, V400F, L408M, I410V, V416M, L417N, Y418Q, M419I, K420R, A423C, T424S, A426I, G429S, I430F, L431M, F433Y, L439M, A440G, A450C, M466R, H477N, A480D, T481K, L485T, G488E, E489G, A491E, Q492E, A493T, H496A, I518V, E522S, M533L, F535A, E540D, S543E, R544E, K545R, A559S, I565M, S567A, Q576N, V578D, D594H, R596K, S611A, C633F, T635Q, K637N, H639D, I641F, L648N, D649N, V651Y, E653S, I658V, V659C, I676G, P677T, R679H, G686K, T692I, E693Q, T697R, V698K, N701H, I703G, L708V, L710M, and A711Y;
k) R5K, M15Y, I17K, I29G, I40K, T41G, D42H, R48V, L53H, D97H, Q180P, T220M, I234T, L242A, D246G, A250N, N251E, S253N, A258D, V260I, G278S, E282N, L285Q, A287Q, H296Q, F297Y, F298N, Q312A, K315V, T316V, I322V, L325F, L334M, F338Y, P348S, T352L, C363D, M365N, P366L, V372C, A383M, A388V, C390M, N399H, L408M, I410V, L411H, V416M, Y418E, M419I, K420A, A423C, T424D, K425S, A426L, A450N, P455S, M466W, N474S, H477N, A480D, T481K, A491F, V494M, S523C, F535A, S543E, R544E, I565M, V578D, Q583N, T598E, V603F, S611A, K614R, R622Q, A625E, L631V, C633F, K637N, I641F, L648N, D649N, V651Y, I658M, V659C, P665A, D666H, I676G, S685K, G686K, E689T, A699R, I703G, Y709V, L710T, and A711V;
l) V2M, R5K, S7T, D14K, M15Y, I17R, L21T, I22M, P23S, A24L, I29G, M30K, R35Y, L36M, T38V, I40K, T41G, D42R, V47I, R48I, Y51W, S52E, L53K, E54T, A55D, Y59L, V60Y, C61K, V62I, H81N, D97Y, D98M, E99K, Q180P, A204D, T220M, I234T, L242A, D246G, A250S, N251E, S253N, A258D, V260I, L271M, G278S, E282N, K283G, L285Q, A287Q, N293D, H296Q, F297Y, F298N, Q312A, K315V, T316V, I322V, L325F, Q327E, L334M, F338Y, K343D, P348H, T352L, F357Y, H360M, D361N, C363D, M365N, P366Y, V372C, R381K, A383M, V392I, L395A, N399H, V400M, L408M, I410V, L411H, D414E, V416M, L417N, Y418Q, M419I, K420R, K422E, A423C, T424D, K425S, A426L, G429S, A440G, A450N, A452K, P455S, G461A, M466W, V471I, N474S, H477N, A480D, T481K, L485N, G488N, E489H, S490K, Q492E, A493D, V494M, S523C, M533L, F535A, L537M, E540D, S543E, R544D, K545R, S547Q, I556V, A559S, I565M, S567A, V578D, Q583N, E597A, T598K, F599Y, H604K, S611A, A615S, P617S, L621M, R622Q, A625E, L631V, C633F, T635Q, K637N, H639D, I641F, R644W, L648N, D649G, V651Y, E653S, I658V, V659C, P665A, D666N, W670I, N674D, I676E, S685K, G686K, F687A, E689I, T692I, E693Q, T697R, V698K, A699Q, I703G, G704A, T705G, I707W, L708V, Y709V, L710M, and A711V;
m) R5K, M15Y, L21T, P23S, A24L, I29G, R35Y, E37R, T41G, D42T, P44T, R48I, L53H, V60Y, V62I, Q180P, T220M, L242A, A250N, N251E, S253N, A258D, V260I, G278S, E282N, L285Q, A287Q, H296Q, F297Y, F298N, Q312A, K315V, T316V, I322V, L325F, F338Y, P348H, T352L, E362K, C363D, M365H, V372C, A383M, C390M, N399H, L408M, I410V, L411H, V416M, Y418Q, M419I, A423L, T424D, A426I, P428R, A450N, P455S, H477N, A480D, A491F, V494M, S523C, F535A, S543E, S547Q, I565M, V578D, Q583N, E597A, T598E, H604K, S611A, K614R, R622Q, L631V, C633F, K637N, I641F, L648N, V651Y, V659C, P665A, D666H, I676G, S685K, F687A, E689I, A699R, I703G, Y709V, L710M, and A711V;
n) R5K, M15Y, L21T, P23S, I29G, R35Y, E37R, T41G, D42T, P44T, R48I, L53H, V60Y, V62I, D97W, Q180P, T220M, L242A, A250N, N251E, S253N, A258D, V260I, E282N, L285Q, A287Q, F297Y, F298N, Q312A, K315V, T316V, I322V, L325F, L334M, F338Y, P348H, T352L, F357Y, E362K, C363D, M365H, V372C, A383M, C390M, N399H, L408M, I410V, L411H, V416M, Y418Q, M419I, A423L, T424D, A450N, P455S, N474S, H477N, A480D, V494M, F535A, S543E, I565M, V578D, Q583N, T598K, V603F, S611A, K614R, R622Q, L631V, C633F, K637N, L648N, V651Y, V659C, H661C, I676G, S685K, F687A, E689I, A699R, I703G, Y709V, L710M, and A711V;
o) M15Y, L21T, I29G, T31S, R35Y, E37R, I40K, T41G, R48I, R76C, L222I, L242A, D246G, N251K, S253N, V260M, G278S, E282N, A287T, F298N, A299V, Q312A, K315I, I322V, L325F, K343I, F357Y, H360Y, C363D, M365N, V372C, A383M, A388I, V392C, L395Q, N399H, L408M, Y418E, K420R, A423C, T424D, A426I, G429S, I430F, L431M, F433Y, F462H, M466R, H477N, L485T, A493D, F535A, S543D, R544E, A559S, V578D, E597S, S611A, C633F, K637N, L648N, D649H, V651Y, I658V, V659C, I676G, N701H, Y709M, and A711Y;
p) R5K, V12L, M15Y, I17K, L21T, I22M, P23S, I29G, R35H, L36M, I40K, T41G, D42R, R48V, Y51W, L53H, E54T, A55D, Y59L, H81N, D97W, L146M, Q180P, T220M, I234T, L242A, D246G, A250S, N251E, S253N, A258D, V260I, G278S, E282N, L285Q, A287Q, H296Q, F297Y, F298N, Q312A, K315V, T316V, I322V, L325F, L334M, F338Y, K343S, P348N, T352L, H360M, C363D, M365N, P366L, V372C, A383M, L395A, N399H, V400M, L408M, I410V, L411H, Y418Q, M419I, K420R, A423C, T424D, K425S, A426L, G429S, A440N, A450N, P455S, M466W, N474S, H477N, A480D, T481K, L485N, G488N, E489H, Q492E, V494M, S523C, M533L, F535A, L537M, S543D, R544D, K545R, A559S, I565M, S567A, V578N, Q583N, E597A, T598K, V603F, H604K, S611A, R622Q, A625E, L631V, C633F, T635Q, K637N, H639D, L640V, L648N, D649N, V651Y, E653S, I658V, V659C, H661C, P665A, D666H, W670I, I676G, S685K, G686K, F687A, E689I, T692I, E693Q, V698K, A699R, I703G, Y709V, L710T, and A711V;
q) R5K, M15Y, I17K, I29G, R35Y, T41G, D42H, R48I, L53H, D97W, Q180P, T220M, L242A, A250N, N251E, S253N, A258D, V260I, E282N, L285Q, A287Q, F297Y, F298N, Q312A, K315V, T316V, I322V, L325F, L334M, F338Y, P348H, T352L, E362K, C363D, M365H, V372C, A383M, A388V, N399H, L408M, I410V, L411H, V416M, Y418Q, M419I, A423L, T424D, A426I, P428R, A450N, P455S, N474S, H477N, A480D, A491F, V494M, S523C, F535A, S543E, R544D, I565M, V578D, Q583N, E597A, T598E, V603F, H604K, S611A, R622Q, A625E, L631V, C633F, K637N, I641F, L648N, D649N, V651Y, I658M, V659C, P665A, D666H, I676G, S685K, F687A, E689I, A699R, I703G, Y709V, L710T, and A711V;
r) M15Y, L21T, P23S, I29G, T31S, R35Y, T41G, R48I, L53Y, R76C, L222I, L242A, N251K, S253N, V260M, G278S, E282N, A287T, F298N, A299V, Q312A, K315I, I322V, L325F, K343I, F357Y, F358Y, H360Y, C363D, M365N, V372C, A383M, A388I, V392T, L395Q, N399H, L408M, Y418E, K420R, A423C, T424D, A426I, I430F, L431M, M466R, L485T, A493D, F535I, S543D, R544E, V578D, E597S, S611A, K637N, L648N, D649H, V651Y, I658V, V659C, I676G, and A711I;
s) M15Y, L21T, P23S, I29G, T31S, R35Y, I40K, T41G, R48I, L53Y, R76C, L222I, L242A, D246G, S253N, V260M, G278S, E282N, A287T, F298N, A299V, Q312A, K315I, I322V, L325F, K343I, F357Y, H360Y, C363D, M365N, V372C, A383M, V392T, L395Q, N399H, L408M, Y418E, K420R, A423C, T424D, A426I, G429S, I430F, L431M, F433Y, F462H, M466R, H477N, L485T, A493D, F535A, R544E, A559S, V578D, S611A, C633F, K637N, L648N, D649H, V651Y, I658V, V659C, I676G, N701H, Y709M, and A711Y;
t) R5K, M15Y, I17K, I29G, I40K, T41G, D42H, L45K, R48V, L53H, D97W, Q180P, T220M, I234T, L242A, D246G, N251E, S253N, A258D, V260I, E282N, L285Q, A287Q, F297Y, F298N, Q312A, K315V, T316V, I322V, L325F, L334M, F338Y, P348S, T352L, E362K, C363D, M365N, P366L, V372C, A383M, A388V, V392L, N399H, L408M, I410V, V416M, Y418Q, M419I, K420A, A423C, T424D, K425S, A426L, A450N, P455S, M466W, H477N, A480D, T481K, V494M, F535A, S543E, R544D, I565M, V578N, Q583N, E597A, T598E, H604K, S611A, K614R, R622Q, A625E, L631V, C633F, K637N, I641F, L648N, D649N, V651Y, I658M, V659C, P665A, D666H, I676G, S685K, G686K, E689T, A699R, I703G, Y709V, L710T, and A711V;
u) R5K, V12L, M15Y, I17K, L21T, I22M, P23S, I29G, R35Y, L36M, I40K, T41G, D42R, R48I, Y51W, L53H, E54T, A55D, Y59L, V60Y, V62I, H81N, D97W, T220M, I234T, L242A, D246G, A250S, N251E, S253N, A258D, V260I, E282N, L285Q, A287Q, F297Y, F298N, A299V, Q312A, K315V, T316V, I322V, L325F, L334M, F338Y, K343S, P348N, T352L, H360M, C363D, M365N, P366L, V372C, A383M, A388V, V392L, L395A, N399H, V400M, L408M, I410V, L411H, V416M, Y418Q, M419I, K420R, E421R, A423C, T424D, K425S, A426L, G429S, A440N, A450N, P455S, G461H, M466W, N474S, H477N, A480D, T481K, L485N, G488N, E489H, Q492E, V494M, S523C, M533L, F535A, L537M, S543D, R544E, K545R, L558T, A559S, I565M, S567A, V578N, Q583N, E597A, T598E, V603F, H604K, S611A, P617S, R622Q, A625E, L631V, C633F, T635Q, K637N, H639D, I641F, L648N, D649N, V651Y, E653S, I658V, V659C, P665A, D666H, W670I, I676G, S685K, G686K, F687A, E689I, T692I, E693Q, V698K, A699R, I703G, Y709V, L710M, and A711V;
v) M15Y, I17K, L21T, P23S, A24L, I29G, T31S, R35Y, E37K, I40K, T41G, D42H, R48I, L53Y, V60F, R76C, H81N, L222I, L242A, D246G, S253N, A258S, V260M, G278S, E282N, A287Q, F297W, F298N, A299V, Q312A, K315I, I322V, L325F, K343I, F357Y, H360Y, C363D, M365N, P366L, V372C, A383M, A388I, V392T, L395A, N399H, L408M, I410V, V416M, Y418E, A423C, T424D, A426I, G429S, I430F, L431M, F433Y, A440N, F462H, M466R, H477N, A480D, T481K, L485T, G488N, Q492K, A493D, F535A, S543D, R544E, A559S, I565V, Q576N, V578D, D594H, R596K, S611A, C633F, T635E, K637N, H639D, L648N, D649H, V651Y, I658V, V659C, I676G, G686K, E689I, T692I, V698K, L710M, and A711Y;
w) M15Y, I17K, L21T, P23S, A24L, I29G, R35Y, I40K, T41G, D42R, L45K, R48V, L53H, E54T, A55E, Y59L, V60Y, V62I, D97H, Q180P, T220M, I234T, L242A, D246G, A250S, N251E, S253N, A258D, V260I, E282N, L285Q, A287Q, F297Y, F298N, Q312A, K315V, T316V, I322V, L325F, L334M, F338Y, K343S, P348N, T352L, C363D, M365N, P366L, V372C, A383M, A388I, V392L, L395A, N399H, L408M, I410V, L411H, V416M, Y418Q, M419I, K420A, A423C, T424D, K425S, A426L, G429S, I430L, A450N, P455S, M466W, N474S, H477N, A480D, T481K, L485N, A491F, V494M, F535A, S543E, R544D, S547Q, I565M, V578N, Q583N, E597A, T598E, H604K, S611A, R622Q, A625E, L631V, C633F, K637N, H639D, I641F, L648N, D649N, V651Y, E653S, V659C, P665A, D666H, I676G, S685K, G686K, F687A, E689I, T692I, E693Q, V698K, A699R, V700L, I703G, Y709V, L710T, and A711V;
x) R5K, M15Y, I17K, L21T, P23S, A24L, I29G, R35Y, E37R, T41G, D42H, P44T, R48I, L53H, V60Y, V62I, D97W, L146M, Q180P, T220M, L242A, A250N, N251E, S253N, A258D, V 260I, E282N, L285Q, A287Q, F297Y, F298N, Q312A, K315V, T316V, I322V, L325V, L334M, F338Y, P348H, T352L, C363D, M365N, V372C, A383M, C390M, N399H, L408M, I410V, L411H, V416M, Y418Q, M419I, A423L, T424D, A426I, P428R, A450N, P455S, N474S, H477N, A480D, A491F, V494M, F535A, S543E, R544D, I565M, V578D, Q583N, E597A, T598E, V603F, H604K, S611A, K614R, R622Q, A625E, L631V, C633F, K637N, I641F, L648N, D649N, V651Y, V659C, P665A, D666H, I676G, S685K, F687A, E689I, A699R, I703G, Y709V, L710M, and A711V;
y) R5K, M15Y, I17R, L21T, P23S, I29G, R35Y, L36M, I40K, T41G, D42R, V47I, R48I, Y51W, L53K, E54T, A55T, Y59L, V60Y, C61R, V62I, H81N, D97H, Q180P, A204D, T220M, I234T, L242A, D246G, A250S, N251E, S253N, A258D, V260I, G278S, E282N, L285Q, A287Q, H296Q, F297Y, F298N, A299V, Q312A, K315V, T316V, I322V, L325F, L334M, F338Y, K343S, P348N, T352L, H360M, C363D, M365N, P366L, V372C, A383M, A388V, L395A, N399H, V400M, L408M, I410V, L411H, Y418Q, M419I, K420A, A423C, T424D, K425S, A426L, G429S, A440N, A450N, P455S, M466W, N474S, H477N, A480D, T481K, L485N, G488E, E489H, Q492E, V494M, M533L, F535A, L537M, E540D, S543E, R544D, K545R, S547A, A559S, I565M, S567A, V578D, Q583N, E597A, T598E, H604K, S611A, A615S, L621M, R622Q, A625E, L631V, C633F, T635Q, K637E, H639D, I641F, A642G, L648N, D649G, V651Y, E653S, I658M, V659C, W670I, N674D, I676G, S685K, G686K, F687A, E689I, T692I, E693Q, V698K, A699R, I703G, T705G, I707W, Y709V, L710M, and A711V;
z) M15Y, L21T, I29G, T31S, R35Y, T41G, R48I, R76C, L222I, L242A, S253N, V260M, G278S, E282N, A287T, F298N, A299V, Q312A, K315I, I322V, L325F, K343I, F357Y, H360Y, C363D, M365N, V372C, A383M, A388I, V392T, L395Q, N399H, L408M, Y418E, K420R, A423C, T424D, A426I, I430F, L431M, F462H, M466R, G488N, Q492K, A493D, F535I, S543D, R544E, V578D, S611A, K637N, L648N, D649H, V651Y, I658V, V659C, I676G, E689I, N701H, Y709M, and A711I;
aa) D14K, M15Y, I17K, I29G, T31S, R35Y, E37K, I40K, T41G, D42H, L45E, A46T, R48V, L53N, A55E, R76C, H81N, L222I, L242A, D246G, A250S, N251K, S253N, A258S, V260M, G278S, E282N, K283G, A287Q, F297Y, F298N, A299V, H309A, Q312A, K315I, I322V, L325F, K343I, P348S, F357Y, H360Y, C363D, M365N, P366L, V372C, A383M, A388V, V392T, L395Q, N399H, V400F, L408M, I410V, V416M, L417N, Y418Q, M419I, K420R, A423C, T424D, A426I, G429S, I430F, L431M, F433Y, L439M, A440G, A450C, M466R, H477N, A480D, T481K, G488N, E489G, A491E, Q492E, A493T, H496A, I518V, E522S, M533L, F535A, E540D, S543D, R544E, K545R, A559S, S567A, Q576N, V578D, D594H, R596K, S611A, C633F, T635Q, K637D, H639D, I641F, L648N, D649N, V651Y, E653S, I658V, V659C, I676G, P677T, R679H, G686K, E689I, T692I, E693Q, T697R, V698K, N701H, I703G, L708V, L710M, and A711Y; or
bb) I17K, I29G, T31S, R35Y, E37K, I40K, T41G, D42H, A46T, R48V, L53Y, R76C, H81N, L222I, L242A, D246G, S253N, A258S, V260M, G278S, E282N, A287Q, F297Y, F298N, A299V, Q312A, K315I, I322V, L325F, K343I, F357Y, F358Y, H360Y, C363D, M365N, P366L, V372C, A383M, V391C, V392T, L395Q, N399H, L408M, I410V, V416M, Y418E, K420R, A423C, T424S, A426I, G429S, I430F, L431M, F433Y, A440N, F462H, M466R, H477N, A480D, T481K, G488N, Q492K, A493D, F535A, R544E, A559S, Q576N, V578D, D594H, R596K, S611A, C633F, T635E, K637N, H639D, L648N, D649H, V651Y, I658V, V659C, I676G, G686K, E689I, T692I, V698K, L710M, and A711Y.

The present invention also provides a pullulanase variant of a wild-type pullulanase from Klebsiella pneumoniae of SEQ ID NO: 11 comprising one or more mutations selected from V66A, R71N, D105C, T122A, T122S, I142C, S225N, D230N, N232E, T236D, T236S, T246S, V247L, Q250E, V251Q, S252A, M253E, R254K, P262A, K265R, V270T, V270K, N271D, S284A, S284T, E286N, V302A, A313S, R328H, Q336N, E338V, V341I, A344T, K347Q, V349I, S358N, A362S, D370E, K372H, A374D, A374K, M380V, M380L, Q386Y, K389N, G424N, G427N, G427S, T429I, K435N, T444A, A462E, E463D, L464W, Q474D, Q474N, E475N, N477A, V490T, V490T, I493V, E523C, I524V, E532S, G535S, D538C, V543I, Q552K, K556S, K556S, T566A, L567A, L567Q, Q570A, Q570G, T571N, Y574F, F581Y, D593N, P594A, T597G, K601A, K601T, T605Q, T605A, I607V, D613N, P632Y, T653V, S656G, A660N, S664C, I677M, V683T, T685A, D687E, I690F, Y700H, Q705N, Q705H, L707N, S708E, S708A, W710R, L771Q, Q773S, P785A, E787D, T789S, L791T, D793Q, L805A, D813S, F814Y, L816F, I817T, K819N, K819S, A822N, V823E, K824V, K825R, G826A, G826S, S827A, E828Q, I829L, A834Q, A834T, G836A, V845A, M860A, M860I, I861L, S862A, A865T, E868D, A869T, L871M, T873D, A879T, S881A, L882A, V885A, Q896H, V931K, R935G, I945L, A954V, E958Q, L961M, K962Q, M964A, T965A, D985S, A987R, T988E, T998A, T1000A, T1004Q, M1015S, Q1016R, S1020D, A1031V, A1031V, T1047N, Q1069R, A1072E, E1087V, E1092G, and A1096T.

The present invention also provides a pullulanase variant of a wild-type pullulanase from Klebsiella pneumoniae of SEQ ID NO: 11 comprising mutations:
a) V66A, R71N, D105C, T122A, I142C, S225N, D230N, N232E, T236D, T246S, V247L, Q250E, V251Q, S252A, M253E, R254K, P262A, K265R, V270T, N271D, S284A, E286N, V302A, A313S, R328H, E338V, V341I, A344T, K347Q, V349I, S358N, A362S, K372H, A374D, M380V, K389N, G424N, G427N, K435N, T444A, A462E, E463D, L464W, Q474D, E475N, N477A, V490T, I493V, E523C, E532S, G535S, D538C, V543I, K556S, T566A, L567A, Q570A, T571N, Y574F, F581Y, P594A, T597G, T605Q, I607V, D613N, P632Y, T653V, S656G, A660N, S664C, I677M, V683T, T685A, D687E, I690F, Y700H, Q705N, S708E, W710R, L771Q, Q773S, P785A, E787D, T789S, L791T, D793Q, L805A, D813S, F814Y, L816F, I817T, K819N, A822N, V823E, K824V, K825R, G826A, S827A, E828Q, I829L, A834Q, G836A, V845A, M860A, I861L, S862A, A865T, E868D, A869T, L871M, T873D, A879T, S881A, L882A, V885A, Q896H, V931K, R935G, I945L, A954V, E958Q, L961M, K962Q, M964A, T965A, D985S, A987R, T988E, T1000A, T1004Q, M1015S, Q1016R, S1020D, A1031V, T1047N, Q1069R, A1072E, E1087V, E1092G, and A1096T;
b) V66A, R71N, D105C, T122A, I142C, S225N, D230N, N232E, T236D, T246S, V247L, Q250E, V251Q, S252A, R254K, P262A, K265R, V270T, N271D, S284A, E286N, V302A, R328H, E338V, V341I, A344T, K347Q, V349I, S358N, A362S, D370E, K372H, A374D, M380V, K389N, G424N, G427N, T429I, K435N, T444A, A462E, E463D, L464W, Q474D, E475N, N477A, V490T, I493V, E523C, E532S, G535S, D538C, V543I, Q552K, K556S, T566A, L567A, Q570A, T571N, Y574F, F581Y, P594A, T597G, K601A, T605Q, I607V, D613N, P632Y, T653V, S656G, A660N, S664C, I677M, V683T, T685A, D687E, I690F, Y700H, Q705N, S708E, W710R, L771Q, Q773S, P785A, E787D, T789S, L791T, D793Q, L805A, D813S, F814Y, L816F, I817T, K819N, A822N, V823E, K824V, K825R, G826S, S827A, E828Q, I829L, A834Q, G836A, V845A, M860A, I861L, S862A, A865T, E868D, A869T, L871M, T873D, A879T, S881A, L882A, V885A, Q896H, V931K, R935G, I945L, E958Q, L961M, K962Q, M964A, T965A, D985S, A987R, T988E, T998A, T1000A, T1004Q, M1015S, Q1016R, S1020D, A1031V, T1047N, Q1069R, A1072E, E1087V, E1092G, and A1096T;
c) D105C, T122A, I142C, S225N, D230N, T236D, T246S, Q250E, V251Q, S252A, M253E, R254K, P262A, V270T, N271D, S284A, V302A, E338V, V341I, A344T, V349I, S358N, K372H, A374K, M380V, Q386Y, G424N, G427S, K435N, A462E, L464W, Q474D, E475N, N477A, V490T, I493V, E523C, E532S, D538C, K556S, T566A, L567Q, Q570A, Y574F, P594A, T597G, K601A, T605Q, D613N, P632Y, T653V, S656G, A660N, S664C, T685A, I690F, Y700H, Q705N, S708E, W710R, L771Q, Q773S, E787D, T789S, D793Q, L805A, L816F, I817T, K819S, A822N, V823E, K824V, K825R, G826A, S827A, E828Q, I829L, A834Q, G836A, M860A, S862A, E868D, A869T, T873D, L882A, Q896H, V931K, R935G, I945L, A954V, E958Q, L961M, K962Q, M964A, D985S, T988E, T1000A, T1004Q, M1015S, Q1016R, S1020D, A1031V, T1047N, E1087V, E1092G, and A1096T;
d) D105C, I142C, T236S, S284T, V341I, V349I, E523C, I524V, D538C, S664C, Q705H, K825R, and T1000A;
e) D105C, I142C, S225N, T236D, T246S, S252A, P262A, N271D, S284A, V341I, A344T, V349I, S358N, K435N, A462E, L464W, Q474D, N477A, V490T, E523C, I524V, D538C, Q552K, K556S, L567A, Q570A, Y574F, P594A, T597G, P632Y, S656G, S664C, T685A, I690F, Y700H, Q705N, L707N, S708E, W710R, L771Q, Q773S, D793Q, L805A, L816F, I817T, K819S, A822N, V823E, K824V, K825R, S827A, I829L, G836A, M860I, S862A, T873D, L882A, R935G, E958Q, L961M, K962Q, D985S, T988E, T1000A, M1015S, S1020D, A1031V, E1087V, and E1092G;
f) D105C, I142C, S225N, D230N, T236D, T246S, V251Q, S252A, P262A, K265R, V270K, N271D, S284A, Q336N, V341I, A344T, V349I, S358N, K435N, A462E, L464W, Q474D, E475N, N477A, V490T, E523C, I524V, E532S, D538C, Q552K, K556S, T566A, L567Q, Q570A, Y574F, D593N, P594A, T597G, P632Y, S656G, A660N, S664C, T685A, I690F, Y700H, Q705N, L707N, S708E, W710R, L771Q, Q773S, T789S, D793Q, L805A, L816F, I817T, K819S, A822N, V823E, K824V, K825R, S827A, I829L, A834T, G836A, M860A, S862A, A869T, T873D, L882A, R935G, E958Q, L961M, K962Q, M964A, D985S, T988E, T1000A, M1015S, S1020D, A1031V, E1087V, and E1092G;
g) D105C, T122A, I142C, S225N, D230N, T236D, T246S, Q250E, V251Q, S252A, M253E, R254K, P262A, K265R, V270T, N271D, S284A, V302A, Q336N, E338V, V341I, A344T, V349I, S358N, K372H, A374K, M380V, G424N, G427S, K435N, A462E, L464W, Q474D, E475N, N477A, V490T, I493V, E523C, E532S, D538C, K556S, T566A, L567A, Q570A, Y574F, P594A, T597G, K601T, T605Q, D613N, P632Y, T653V, S656G, A660N, S664C, T685A, I690F, Y700H, Q705N, L707N, S708E, W710R, L771Q, Q773S, E787D, T789S, D793Q, L805A, L816F, I817T, K819S, A822N, V823E, K824V, K825R, G826A, S827A, E828Q, I829L, A834Q, G836A, M860A, S862A, E868D, A869T, T873D, L882A, Q896H, V931K, R935G, I945L, A954V, E958Q, L961M, K962Q, M964A, D985S, T988E, T1000A, T1004Q, M1015S, Q1016R, S1020D, A1031V, T1047N, E1087V, E1092G, and A1096T;
h) D105C, T122S, I142C, S225N, D230N, T236D, T246S, V251Q, S252A, P262A, V270T, N271D, S284A, E338V, V341I, A344T, V349I, S358N, M380L, G427S, K435N, A462E, L464W, Q474D, E475N, N477A, V490T, E523C, E532S, D538C, Q552K, K556S, T566A, L567Q, Q570A, Y574F, P594A, T597G, T605A, P632Y, S656G, A660N, S664C, T685A, I690F, Y700H, Q705N, S708E, W710R, L771Q, Q773S, E787D, T789S, D793Q, L805A, L816F, I817T, K819S, A822N, V823E, K824V, K825R, S827A, I829L, A834T, G836A, M860A, S862A, E868D, A869T, T873D, L882A, V931K, R935G, A954V, E958Q, L961M, K962Q, M964A, D985S, T988E, T1000A, M1015S, Q1016R, S1020D, A1031V, T1047N, E1087V, and E1092G;
i) D105C, I142C, S225N, T236S, S252A, P262A, S284A, V341I, V349I, K435N, A462E, N477A, E523C, I524V, D538C, L567A, Q570G, Y574F, P594A, T597G, P632Y, S656G, S664C, T685A, I690F, Q705N, L707N, S708E, Q773S, D793Q, L805A, K819S, V823E, K824V, K825R, S827A, G836A, R935G, L961M, K962Q, D985S, T988E, T1000A, M1015S, S1020D, and A1031V;
j) D105C, I142C, S225N, T236S, T246S, S252A, P262A, N271D, S284A, V341I, V349I, K435N, A462E, L464W, Q474N, N477A, V490T, E523C, I524V, D538C, Q552K, L567Q, Y574F, P594A, T597G, P632Y, S656G, S664C, T685A, I690F, Y700H, Q705N, L707N, S708E, L771Q, Q773S, D793Q, L805A, K819S, V823E, K824V, K825R, S827A, G836A, M860I, S862A, L882A, R935G, L961M, K962Q, D985S, T988E, T1000A, M1015S, S1020D, A1031V, and E1087V;
k) D105C, I142C, S225N, D230N, T236D, T246S, V251Q, S252A, P262A, V270K, N271D, S284A, V341I, A344T, V349I, S358N, K435N, A462E, L464W, Q474D, E475N, N477A, V490T, E523C, I524V, E532S, D538C, K556S, T566A, L567Q, Q570A, Y574F, P594A, T597G, K601A, P632Y, S656G, A660N, S664C, T685A, I690F, Y700H, Q705N, S708E, W710R, L771Q, Q773S, T789S, D793Q, L805A, L816F, I817T, K819S, A822N, V823E, K824V, K825R, S827A, I829L, A834T, G836A, M860A, S862A, A869T, T873D, L882A, R935G, E958Q, L961M, K962Q, M964A, D985S, T988E, T1000A, M1015S, S1020D, A1031V, E1087V, and E1092G;
l) D105C, T122S, I142C, S225N, D230N, T236D, T246S, V251Q, S252A, P262A, V270T, N271D, S284A, Q336N, E338V, V341I, A344T, V349I, S358N, M380L, G427S, K435N, A462E, L464W, Q474D, E475N, N477A, V490T, E523C, E532S, D538C, K556S, T566A, L567A, Q570A, Y574F, P594A, T597G, T605A, P632Y, S656G, A660N, S664C, T685A, I690F, Y700H, Q705N, L707N, S708E, W710R, L771Q, Q773S, E787D, T789S, D793Q, L805A, L816F, I817T, K819S, A822N, V823E, K824V, K825R, S827A, I829L, A834T, G836A, M860A, S862A, E868D, A869T, T873D, L882A, V931K, R935G, E958Q, L961M, K962Q, M964A, D985S, T988E, T1000A, M1015S, Q1016R, S1020D, A1031V, T1047N, E1087V, and E1092G; or
m) D105C, I142C, S225N, T236S, S252A, P262A, S284A, V341I, V349I, K435N, A462E, N477A, E523C, I524V, D538C, Q552K, L567A, Q570G, Y574F, P594A, T597G, P632Y, S656G, S664C, T685A, I690F, Q705N, L707N, S708A, Q773S, D793Q, L805A, K819S, V823E, K824V, K825R, S827A, G836A, R935G, L961M, K962Q, D985S, T988E, T1000A, M1015S, S1020D, and A1031V.

The present invention provides a transglutaminase variant of a wild-type transglutaminase from Streptomyces mobaraensis of SEQ ID NO: 12 comprising one or more mutations selected from D54A, P98A, S102R, S102Y, K128A, Q153P, F163S, D167N, K174E, G176T, A199R, A199E, Q203E, Q203K, D227K, K231A, A234G, N235K, G236K, G236N, A245S, K260N, D261N, S283G, S291D, S325G, G327N, D379E, D379A, S382A, A388T, V390I, V390M, V391I, V394I, D403A, K404E, and T406K.

The present invention provides a transglutaminase variant of a wild-type transglutaminase from Streptomyces mobaraensis of SEQ ID NO: 12 comprising mutations:
a) P98A, S102R, K128A, Q153P, G236K, K260N, D261N, S283G, S325G, D379E, S382A, A388T, V391I, V394I, and T406K;
b) D54A, P98A, S102R, K128A, Q153P, G176T, Q203E, A234G, G236K, K260N, S283G, S325G, D379E, S382A, A388T, V390I, V391I, V394I, and T406K;
c) P98A, S102R, K128A, Q153P, G176T, Q203E, G236K, K260N, S283G, S325G, D379E, S382A, A388T, V391I, V394I, and T406K;
d) D54A, P98A, S102R, K128A, Q153P, F163S, D167N, K174E, G176T, A199R, Q203E, D227K, K231A, A234G, G236K, A245S, K260N, D261N, S283G, S291D, S325G, D379A, S382A, A388T, V391I, V394I, D403A, K404E, and T406K;
e) D54A, P98A, S102R, K128A, Q153P, D167N, K174E, G176T, D227K, A234G, K260N, D261N, S283G, S325G, D379A, S382A, A388T, V390I, V391I, V394I, and T406K;
f) D54A, P98A, S102Y, K128A, Q153P, A234G, N235K, G236K, K260N, S283G, S325G, D379E, S382A, A388T, V390I, V391I, V394I, and T406K;
g) D54A, P98A, S102R, K128A, Q153P, Q203E, A234G, N235K, G236N, K260N, S283G, S325G, D379E, S382A, A388T, V391I, V394I, and T406K;
h) P98A, S102R, K128A, Q153P, F163S, D167N, K174E, G176T, A199E, Q203K, D227K, K231A, G236K, A245S, K260N, S283G, S291D, S325G, D379A, S382A, A388T, V391I, V394I, D403A, and T406K;
i) P98A, S102R, K128A, Q153P, F163S, D167N, K174E, G176T, A199R, Q203E, D227K, K231A, G236K, A245S, K260N, S283G, S291D, S325G, D379A, S382A, A388T, V390M, V391I, V394I, D403A, and T406K;
j) D379E, V391I, V394I, and T406K;
k) P98A, S102Y, K128A, Q153P, G236K, K260N, D261N, S283G, S325G, D379E, S382A, A388T, V391I, V394I, and T406K;
l) D54A, P98A, S102R, K128A, Q153P, D167N, K174E, G176T, Q203E, D227K, N235K, G236K, K260N, S283G, S325G, D379A, S382A, A388T, V391I, V394I, and T406K;
m) P98A, S102Y, K128A, Q153P, A234G, G236K, K260N, S283G, S325G, D379E, S382A, A388T, V390I, V391I, V394I, and T406K;
n) D54A, P98A, S102Y, K128A, Q153P, F163S, D167N, K174E, G176T, A199E, Q203K, D227K, K231A, G236K, A245S, K260N, S283G, S291D, S325G, G327N, D379A, S382A, A388T, V391I, V394I, D403A, and T406K; or
o) D54A, P98A, S102Y, K128A, Q153P, A234G, G236K, K260N, S283G, S325G, D379E, S382A, A388T, V391I, V394I, and T406K.

The present invention provides a Xaa-Pro dipeptidase variant of a wild-type dipeptidase from Alteromonas sp. of SEQ ID NO: 13 comprising one or more mutations selected from N2D, K3Q, V6H, V6A, E10Q, I12L, K17R, K17Q, T19Y, R20D, E24K, D29S, D29A, V31Y, F33I, Q55H, I63V, A72V, T75G, E98D, A101T, D102R, D102E, D102K, D102S, A124S, A138W, E142Q, L143H, L143Q, M144V, M144I, M144R, F152W, Q162D, K173A, Q177K, G178A, D184E, D184K, S191Y, Q197A, L201R, Q204G, S206H, N210M, N210T, N221S, I224V, F230Y, V233Q, A236P, T237E, I243F, A247G, F249Y, T263H, E265Q, E265H, V272I, A273N, T274A, K276H, H278M, I280Q, N284A, L286M, L291W, G293V, L297E, D298Q, D298E, C299M, C299A, V303I, T306L, D309E, N311G, I312L, I312F, D318E, I320Q, F348W, M349Q, Q353R, A355T, K371I, I372V, A374P, Q376M, S389M, G392A, G392E, D393K, D393Q, D393E, D393R, A395R, A395K, T397G, T397Q, T397S, D398E, N399H, N400A, H402M, K412R, F414Y, and I424C.

The present invention provides a Xaa-Pro dipeptidase variant of a wild-type dipeptidase from Alteromonas sp. of SEQ ID NO: 13 comprising mutations:
a) I12L, T19Y, E24K, F33I, A72V, T75G, A101T, D102R, F152W, Q162D, K173A, Q197A, N210M, V233Q, A247G, T274A, K276H, I280Q, G293V, T306L, D309E, I312L, A355T, A374P, G392A, D393K, A395R, D398E, N400A, and K412R;
b) V6H, I12L, T19Y, E24K, D29S, F33I, A72V, T75G, A101T, D102E, A138W, E142Q, M144V, F152W, Q162D, K173A, Q197A, N210M, V233Q, A247G, T263H, T274A, K276H, H278M, I280Q, N284A, L291W, G293V, C299M, V303I, T306L, D309E, I312L, D318E, Q353R, A355T, G392A, D393Q, A395R, T397G, D398E, N400A, K412R, and I424C;
c) V6H, I12L, T19Y, E24K, D29S, F33I, A72V, T75G, A101T, D102K, F152W, Q162D, K173A, Q197A, N210M, V233Q, A247G, T274A, K276H, I280Q, L291W, G293V, V303I, T306L, D309E, I312F, D318E, Q353R, A355T, G392A, D393Q, A395K, T397G, D398E, N400A, K412R, and I424C;
d) N2D, K3Q, V6H, E10Q, I12L, K17R, T19Y, E24K, D29A, V31Y, F33I, Q55H, I63V, A72V, T75G, E98D, A101T, D102E, A138W, E142Q, L143H, M144I, F152W, Q162D, K173A, Q177K, G178A, D184E, S191Y, Q197A, L201R, Q204G, S206H, N210T, I224V, F230Y, V233Q, T237E, I243F, A247G, F249Y, T263H, E265Q, V272I, A273N, T274A, K276H, H278M, I280Q, N284A, L286M, G293V, L297E, D298Q, C299M, V303I, T306L, D309E, N311G, I312L, D318E, I320Q, F348W, M349Q, Q353R, A355T, I372V, A374P, Q376M, S389M, G392A, D393E, A395R, T397Q, D398E, N399H, N400A, H402M, K412R, F414Y, and I424C;
e) N2D, V6H, I12L, K17Q, T19Y, E24K, D29S, V31Y, F33I, A72V, T75G, A101T, D102E, A124S, A138W, E142Q, L143Q, M144I, F152W, Q162D, K173A, D184K, S191Y, Q197A N210T, N221S, F230Y, V233Q, A236P, T237E, A247G, F249Y, T263H, E265H, A273N, T274A, K276H, H278M, I280Q, N284A, L286M, L291W, G293V, L297E, D298E, C299A, V303I, T306L, D309E, N311G, I312L, D318E, I320Q, M349Q, Q353R, A355T, K371I, I372V, Q376M, S389M, G392A, D393Q, A395R, T397S, D398E, N400A, K412R, F414Y, and I424C;
f) N2D, K3Q, V6H, E10Q, I12L, K17R, T19Y, E24K, D29S, V31Y, F33I, Q55H, I63V, A72V, T75G, E98D, A101T, D102K, A138W, E142Q, L143H, M144I, F152W, Q162D, K173A, Q177K, G178A, D184E, S191Y, Q197A, L201R, Q204G, S206H, N210T, I224V, F230Y, V233Q, T237E, A247G, F249Y, T263H, E265H, V272I, A273N, T274A, K276H, H278M, I280Q, N284A, L286M, L291W, G293V, L297E, D298Q, C299A, V303I, T306L, D309E, N311G, I312L, I320Q, F348W, M349Q, Q353R, A355T, K371I, I372V, A374P, Q376M, S389M, G392A, D393Q, A395R, T397S, D398E, N399H, N400A, K412R, F414Y, and I424C;
g) V6H, I12L, T19Y, E24K, D29S, F33I, A72V, T75G, A101T, D102K, A138W, E142Q, F152W, Q162D, K173A, Q197A, N210M, V233Q, A247G, T263H, T274A, K276H, H278M, I280Q, N284A, L291W, G293V, C299M, V303I, T306L, D309E, I312L, Q353R, A355T, G392A, D393R, A395R, T397G, D398E, N400A, K412R, and I424C;
h) V6H, I12L, T19Y, E24K, D29A, V31Y, F33I, A72V, T75G, A101T, D102E, A138W, E142Q, M144I, F152W, Q162D, K173A, S191Y, Q197A, N210M, V233Q, T237E, A247G, T263H, E265H, A273N, T274A, K276H, H278M, I280Q, N284A, L291W, G293V, C299M, V303I, T306L, D309E, I312L, D318E, Q353R, A355T, G392A, D393Q, A395R, T397G, D398E, N400A, K412R, and I424C;
i) I12L, T19Y, E24K, F33I, A72V, T75G, A101T, D102S, F152W, Q162D, K173A, Q197A, N210M, V233Q, A247G, T274A, K276H, I280Q, G293V, T306L, D309E, I312L, A355T, K371I, G392A, D393R, A395R, D398E, N400A, and K412R;
j) N2D, V6H, I12L, K17Q, T19Y, E24K, D29S, F33I, A72V, T75G, A101T, D102K, A124S, A138W, E142Q, L143Q, M144R, F152W, Q162D, K173A, D184K, S191Y, Q197A, N210T, N221S, F230Y, V233Q, T237E, A247G, F249Y, T263H, E265H, A273N, T274A, K276H, H278M, I280Q, N284A, L286M, L291W, G293V, L297E, D298E, C299A, V303I, T306L, D309E, N311G, I312L, D318E, I320Q, M349Q, Q353R, A355T, I372V, Q376M, S389M, G392A, D393Q, A395R, T397S, D398E, N400A, H402M, K412R, F414Y, and I424C;
k) V6H, I12L, T19Y, E24K, D29S, F33I, A72V, T75G, A101T, D102K, M144V, F152W, Q162D, K173A, Q197A, N210M, V233Q, A247G, T274A, K276H, I280Q, L291W, G293V, V303I, T306L, D309E, I312L, D318E, Q353R, A355T, K371I, A374P, G392A, D393R, A395R, T397G, D398E, N400A, K412R, and I424C;
l) V6H, I12L, T19Y, E24K, D29S, V31Y, F33I, A72V, T75G, A101T, D102K, A138W, E142Q, F152W, Q162D, K173A, S191Y, Q197A, N210M, V233Q, T237E, A247G, T263H, E265H, A273N, T274A, K276H, H278M, I280Q, N284A, G293V, C299A, V303I, T306L, D309E, I312F, Q353R, A355T, G392A, D393Q, A395R, T397G, D398E, N400A, K412R, and I424C;
m) N2D, V6H, I12L, T19Y, E24K, D29S, V31Y, F33I, A72V, T75G, A101T, D102R, A124S, A138W, E142Q, L143Q, F152W, Q162D, K173A, S191Y, Q197A, N210T, N221S, F230Y, V233Q, T237E, A247G, T263H, E265H, A273N, T274A, K276H, H278M, I280Q, N284A, L291W, G293V, C299M, V303I, T306L, D309E, I312L, Q353R, A355T, G392E, D393Q, A395R, T397G, D398E, N400A, H402M, K412R, F414Y, and I424C; or
n) N2D, V6A, I12L, T19Y, R20D, E24K, D29S, F33I, A72V, T75G, A101T, D102K, A124S, A138W, E142Q, L143Q, M144R, F152W, Q162D, K173A, S191Y, Q197A, N210T, N221S, F230Y, V233Q, T237E, A247G, T263H, E265H, A273N, T274A, K276H, H278M, I280Q, N284A, G293V, C299M, V303I, T306L, D309E, I312L, D318E, Q353R, A355T, G392A, D393Q, A395R, T397G, D398E, N400A, H402M, K412R, F414Y, and I424C.

The present invention provides a xylanase variant of a wild-type xylanase from Aspergillus niger of SEQ ID NO: 14 comprising one or more mutations selected from L43F, T51H, S56T, T58Y, A64R, L69I, S88D, S88N, S90T, S92A, L95V, Q122K, Q143G, S204N, S206N, L210V, T211Q, G243N, S245P, A250R, G253S, G253A, T256V, V261I, S271A, E276T, E276A, E279K, I288V, T306N, N315Q, and T321W.

The present invention provides a xylanase variant of a wild-type xylanase from Aspergillus niger of SEQ ID NO: 14 comprising mutations:
a) L43F, T51H, T58Y, L69I, S88D, L95V, S245P, T256V, E276T, T306N, and T321W;
b) L43F, T51H, S56T, T58Y, L69I, S88D, S92A, L95V, Q143G, T211Q, G243N, S245P, G253S, T256V, V261I, S271A, E276A, E279K, I288V, T306N, and T321W;
c) L43F, T51H, T58Y, L69I, S88D, L95V, Q143G, G243N, S245P, T256V, V261I, S271A, E276A, I288V, T306N, and T321W;
d) L43F, T51H, T58Y, L69I, S88D, L95V, G243N, S245P, T256V, E276T, T306N, and T321W;
e) L43F, T51H, S56T, T58Y, A64R, L69I, S88N, S90T, S92A, L95V, Q122K, Q143G, S206N, L210V, T211Q, G243N, S245P, A250R, G253S, T256V, V261I, S271A, E276A, E279K, I288V, T306N, N315Q, and T321W;
f) L43F, T51H, S56T, T58Y, A64R, L69I, S88N, S90T, S92A, L95V, Q122K, Q143G, S204N, L210V, T211Q, G243N, S245P, A250R, G253A, T256V, V261I, S271A, E276A, E279K, I288V, T306N, N315Q, and T321W;
g) L43F, T51H, T58Y, L69I, S88D, L95V, G243N, S245P, T256V, E276A, I288V, T306N, and T321W; or
h) L43F, T51H, T58Y, L69I, S88D, L95V, G243N, S245P, T256V, E276A, T306N, and T321W.

The present invention also provides a xylanase variant of a wild-type xylanase from Bacillus amyloliquefaciens of SEQ ID NO: 15 comprising one or more mutations selected from D32H, D32N, T43S, T43N, A46W, A46Y, N57T, T61N, T71S, F76N, A83K, V85T, W86Y, A87N, N91G, T95C, A102N, S112N, T121S, N150K, S168G, A171T, K182N, K182Q, L198M, and K204Q.

The present invention also provides a xylanase variant of a wild-type xylanase from Bacillus amyloliquefaciens of SEQ ID NO: 15 comprising mutations:
a) D32H, T43S, A46W, N57T, T71S, F76N, A83K, W86Y, A87N, N91G, T95C, A102N, T121S, N150K, S168G, K182N, L198M, and K204Q;
b) L198M, and K204Q;
c) D32N, T43N, A46W, N57T, T61N, F76N, A83K, V85T, W86Y, A87N, N91G, T95C, A102N, T121S, N150K, S168G, K182Q, L198M, and K204Q;
d) D32H, T43S, A46Y, F76N, A87N, N91G, A102N, S168G, K182N, L198M, and K204Q;
e) D32H, T43S, A46W, F76N, A83K, A87N, N91G, A102N, S168G, K182N, L198M, and K204Q;
f) D32H, T43N, A46W, N57T, F76N, A83K, A87N, N91G, A102N, S168G, K182N, L198M, and K204Q;
g) D32H, T43S, A46W, N57T, F76N, A83K, A87N, N91G, A102N, S168G, K182N, L198M, and K204Q;
h) D32H, T43N, A46Y, N57T, F76N, A83K, W86Y, A87N, N91G, T95C, A102N, S112N, T121S, N150K, S168G, K182N, L198M, and K204Q;
i) D32N, T43S, A46W, N57T, T61N, F76N, A83K, V85T, W86Y, A87N, N91G, T95C, A102N, T121S, N150K, S168G, A171T, K182N, L198M, and K204Q;
j) D32H, T43N, A46W, F76N, A83K, A87N, N91G, A102N, S168G, K182N, L198M, and K204Q;
k) T43S, A46W, F76N, A83K, A87N, N91G, A102N, S168G, K182N, L198M, and K204Q;
l) D32H, T43N, A46W, N57T, F76N, A87N, N91G, A102N, S112N, T121S, S168G, K182N, L198M, and K204Q; or
m) D32H, T43N, A46Y, N57T, F76N, A83K, A87N, N91G, A102N, T121S, S168G, K182Q, L198M, and K204Q.

The present invention further provides a xylanase variant of a wild-type xylanase from Penicillium of SEQ ID NO: 16 comprising one or more mutations selected from K38L, F39L, N49S, N49T, G58K, N69D, F99Y, F102W, T105Q, G107N, I110V, L115T, D136R, K147G, K147S, Q148R, L153C, I162A, F182Y, V183I, R184A, I201V, D203S, T210S, T210A, V229T, V229M, A244G, A247D, A248Q, S249T, G250E, A251K, T260A, E261S, V265I, E267M, A273G, T274A, T274S, T274N, V279A, D280N, L286C, Q288V, V292C, A308S, A308S, D309S, E310S, E310S, S311T, S311T, A317S, S318N, E323P.

The present invention further provides a xylanase variant of a wild-type xylanase from Penicillium of SEQ ID NO: 16 comprising mutations:
a) N49S, G58K, T105Q, D136R, K147G, Q148R, D203S, T210S, V229T, A248Q, S249T, E261S, T274A, V279A, D280N, L286C, V292C, A308S, D309S, E310S, S311T, A317S, S318N, and E323P
b) F39L, N49S, G58K, T105Q, D136R, K147G, Q148R, I162A, D203S, T210A, V229T, A248Q, S249T, E261S, A273G, T274S, V279A, D280N, L286C, Q288V, V292C, A308S, D309S, E310S, S311T, A317S, S318N, and E323P
c) F39L, N49S, G58K, T105Q, L115T, D136R, K147G, Q148R, I162A, T210A, V229T, A248Q, S249T, E261S, E267M, A273G, T274S, V279A, D280N, L286C, Q288V, V292C, A308S, D309S, E310S, S311T, A317S, S318N, and E323P;
d) N49S, G58K, T105Q, D136R, K147G, Q148R, D203S, V229T, A248Q, E261S, T274N, V279A, D280N, L286C, V292C, A308S, D309S, E310S, S311T, A317S, S318N, and E323P;
e) K38L, F39L, N49S, G58K, N69D, F99Y, F102W, T105Q, G107N, I110V, L115T, D136R, K147G, Q148R, L153C, I162A, F182Y, V183I, R184A, I201V, T210A, V229M, A244G, A247D, A248Q, S249T, G250E, A251K, T260A, E261S, V265I, A273G, T274S, V279A, D280N, L286C, Q288V, V292C, A308S, D309S, E310S, S311T, A317S, S318N, and E323P;
f) N49T, G58K, T105Q, L115T, D136R, K147G, Q148R, V229T, A248Q, T274N, D280N, L286C, V292C, A308S, D309S, E310S, S311T, A317S, S318N, and E323P
g) N49S, G58K, T105Q, L115T, D136R, K147G, Q148R, T210S, V229T, A248Q, S249T, E261S, T274N, V279A, D280N, L286C, V292C, A308S, D309S, E310S, S311T, A317S, S318N, and E323P;
h) N49T, G58K, T105Q, D136R, K147S, Q148R, D203S, V229T, A248Q, T274N, D280N, L286C, V292C, A308S, D309S, E310S, S311T, A317S, S318N, and E323P;
i) N49T, G58K, T105Q, L115T, D136R, K147G, Q148R, V229T, A248Q, L286C, V292C, A308S, D309S, E310S, S311T, A317S, S318N, and E323P;
j) K38L, F39L, N49S, G58K, N69D, F99Y, F102W, T105Q, G107N, I110V, D136R, K147G, Q148R, L153C, I162A, F182Y, V183I, R184A, I201V, D203S, T210A, V229T, A244G, A247D, A248Q, S249T, G250E, A251K, T260A, E261S, V265I, A273G, T274S, V279A, D280N, L286C, Q288V, V292C, A308S, D309S, E310S, S311T, A317S, S318N, and E323P;
k) K38L, F39L, N49S, G58K, N69D, F102W, T105Q, G107N, D136R, K147G, Q148R, I162A, F182Y, V183I, R184A, D203S, T210A, V229T, A248Q, S249T, E261S, A273G, T274S, V279A, D280N, L286C, Q288V, V292C, A308S, D309S, E310S, S311T, A317S, S318N, and E323P; or
l) L286C, V292C, A308S, D309S, E310S, S311T, A317S, S318N, and E323P.

The present invention still further provides a xylanase variant of a wild-type xylanase from Talaromyces pinophilus of SEQ ID NO: 29 comprising one or more mutations selected from T68G, S73H, T76N, E79A, E79S, E79T, T84N, P101S, L149V, N161V, N161C, E166Q, S173K, S173R, Q184N, Q184R, E189S, T193N, Q195A, H197F, K202A, N203S, M206L, N207H, T210Q, L211M, F213Y, I216V, A218V, Y229Q, Y229I, A230T, A230S, and N232S.

The present invention still further provides a xylanase variant of a wild-type xylanase from Talaromyces pinophilus of SEQ ID NO: 29 comprising mutations:
a) T68G, E79A, T84N, P101S, L149V, E166Q, S173K, Q184N, E189S, K202A, N203S, M206L, N207H, T210Q, L211M, F213Y, I216V, A218V, Y229Q, A230T, and N232S;
b) T68G, S73H, T76N, E79S, T84N, P101S, L149V, N161V, E166Q, S173R, Q184R, E189S, T193N, Q195A, K202A, N203S, M206L, N207H, L211M, F213Y, I216V, A218V, Y229I, A230T, and N232S;
c) T68G, S73H, E79S, T84N, P101S, L149V, N161V, E166Q, S173R, Q184R, E189S, T193N, Q195A, H197F, K202A, N203S, M206L, N207H, L211M, F213Y, I216V, A218V, Y229I, A230T, and N232S;
d) T68G, P101S, E166Q, Q184N, E189S, K202A, N203S, M206L, N207H, T210Q, L211M, F213Y, I216V, A218V, Y229Q, A230S, and N232S;
e) T68G, E79A, P101S, E166Q, S173R, Q184N, E189S, K202A, N203S, M206L, N207H, T210Q, L211M, F213Y, I216V, A218V, Y229Q, A230S, and N232S;
f) T68G, E79T, T84N, P101S, L149V, E166Q, S173K, Q184N, E189S, K202A, N203S, M206L, N207H, T210Q, L211M, F213Y, I216V, A218V, Y229Q, A230T, and N232S;
g) Q184N, E189S, K202A, N203S, M206L, N207H, T210Q, L211M, F213Y, I216V, A218V, Y229Q, A230S, and N232S
h) T68G, S73H, T76N, E79S, T84N, P101S, L149V, N161V, E166Q, S173R, Q184R, E189S, T193N, Q195A, H197F, K202A, N203S, M206L, N207H, L211M, F213Y, I216V, A218V, Y229I, A230T, and N232S;
i) T68G, S73H, E79S, T84N, P101S, L149V, N161V, E166Q, S173R, Q184R, E189S, K202A, N203S, M206L, N207H, T210Q, L211M, F213Y, I216V, A218V, Y229Q, A230T, and N232S; or
j) T68G, S73H, E79S, T84N, P101S, L149V, N161C, E166Q, S173R, Q184R, E189S, T193N, Q195A, K202A, N203S, M206L, N207H, L211M, F213Y, I216V, A218V, Y229I, A230T, and N232S.

The present invention provides a xylanase variant of a wild-type xylanase from Thermomyces lanuginosus of SEQ ID NO: 30 comprising one or more mutations selected from S69T, S69E, A88N, F124Y, D128N, D135K, I145T, R147D, R147D, A156Q, D160E, Q182T, Q182N, A191R, L195M, and N198G.

The present invention provides a xylanase variant of a wild-type xylanase from Thermomyces lanuginosus of SEQ ID NO: 30 comprising mutations:
a) R147D, A156Q, and Q182T;
b) I145T, R147D, A156Q, Q182T, A191R, and N198G;
c) S69T, A88N, D128N, I145T, R147D, A156Q, Q182T, A191R, and N198G;
d) I145T, R147D, A156Q, Q182T, and N198G;
e) S69T, A88N, F124Y, D128N, D135K, I145T, R147D, A156Q, D160E, Q182T, A191R, L195M, and N198G;
f) S69T, A88N, D128N, I145T, R147D, A156Q, Q182N, A191R, N198G;
g) R147D, A156Q, Q182T, and N198G;
h) S69E, A88N, F124Y, D128N, D135K, I145T, R147D, A156Q, D160E, Q182N, A191R, L195M, and N198G;
i) I145T, R147D, A156Q, Q182N, and N198G; or
j) R147D, A156Q, Q182N, and N198G.

The present invention also provides a xylanase variant of a wild-type xylanase from Bacillus paralicheniformis of SEQ ID NO: 31 comprising one or more mutations selected from A33V, R36A, R36H, R36S, A39T, K41H, V43T, A61P, R64M, E65Q, F68I, H83G, E86P, N90D, N90E, Y92N, K99Q, K99N, S100R, I102A, I102Q, K103S, K103A, K103A, K103E, H104R, H104M, H104L, H104N, I107M, I107K, I107T, V108L, S117A, V102I, N124T, N124S, S130N, S130D, A140Q, K144A, K144Q, H145W, D148S, T151E, F152Y, K154G, K154Q, W177C, I183F, L184A, L184R, M187I, M187L, K188A, N195S, A196C, I199M, I199L, L207N, I210M, P218A, K219T, L227V, L227S, A229T, T234G, Q235S, N237S, G248H, D252E, D252R, D252N, Y260H, S278A, H280E, S284C, D289N, K306W, K306R, T310N, Y316A, N317S, H320Q, Y343C, K348S, K348S, K348T, G349S, D350G, V353I, K360N, G364Q, G364A, G364T, N366T, N368T, N368Q, S375T, S375T, S377T, S380T, I383M, L390M, Q391A, G393T, N396D, N398T, T400S, D401G, H403Y, H403R, H407W, H407N, H407Y, A418V, and A418I.

The present invention also provides a xylanase variant of a wild-type xylanase from Bacillus paralicheniformis of SEQ ID NO: 31 comprising mutations:
a) A33V, K99Q, S100R, I102A, K103S, I107M, S130N, A140Q, W177C, M187I, K188A, L207N, I210M, L227V, D252E, S284C, D289N, K306W, Y316A, K348S, K360N, G364Q, N368T, S375T, L390M, G393T, N398T, T400S, D401G, H407W, and A418V;
b) K99Q, S100R, I102A, I107M, A140Q, T151E, L207N, L227V, D252R, S284C, Y316A, K348S, K360N, L390M, N398T, T400S, D401G, H407W, and A418V;
c) A33V, K99Q, S100R, I102A, K103S, I107M, S130N, A140Q, W177C, M187I, K188A, L207N, I210M, L227V, D252E, S284C, D289N, K348S, K360N, G364Q, N368T, S375T, L390M, G393T, N398T, T400S, D401G, H407W, and A418V;
d) A33V, E65Q, Y92N, K99Q, S100R, K103A, I107K, V108L, N124T, K144A, K188A, I199M, L207N, K219T, L227V, D252R, H280E, S284C, K306R, T310N, K348S, D350G, L390M, G393T, N398T, T400S, D401G, H407W, and A418V;
e) A33V, R36A, R64M, E65Q, E86P, N90D, K99Q, S100R, K103A, H104R, I107K, V108L, V102I, N124T, S130D, K144A, F152Y, L184A, M187I, K188A, A196C, I199M, L207N, I210M, K219T, L227V, T234G, N237S, G248H, D252R, S278A, H280E, S284C, D289N, K306R, T310N, K348S, D350G, V353I, N368T, L390M, G393T, N396D, N398T, T400S, D401G, H403Y, H407W, and A418V;
f) A33V, E65Q, Y92N, K99Q, S100R, K103A, I107K, V108L, N124T, K144A, K188A, I199M, L207N, K219T, L227V, D252R, H280E, S284C, K306R, T310N, K348S, D350G, N368Q, L390M, G393T, N398T, T400S, D401G, H407W, and A418V;
g) H83G, K99Q, S100R, I102Q, K103A, A140Q, W177C, L207N, L227V, D252E, S284C, D289N, K348S, K360N, S375T, L390M, G393T, N398T, T400S, D401G, H407W, and A418V;
h) K99Q, S100R, I102A, K103A, A140Q, K154G, W177C, L207N, L227V, D252N, S284C, D289N, Y316A, K348S, K360N, S375T, L390M, G393T, N398T, T400S, D401G, H407W, and A418V;
i) K99Q, S100R, I102A, K103A, I107M, A140Q, W177C, L207N, L227V, D252N, S284C, Y316A, K348T, K360N, S377T, L390M, G393T, N398T, T400S, D401G, H407W, and A418V;
j) A33V, R36H, A39T, K41H, V43T, H83G, N90E, K99N, S100R, I102Q, K103A, H104M, I107T, S117A, N124S, S130N, A140Q, K144Q, F152Y, K154Q, W177C, I183F, L184R, M187L, K188A, A196C, L207N, I210M, P218A, K219T, L227V, A229T, T234G, N237S, G248H, D252R, Y260H, S278A, S284C, D289N, T310N, N317S, H320Q, Y343C, K348S, G349S, D350G, K360N, G364A, N368T, S375T, S380T, I383M, L390M, Q391A, G393T, N396D, N398T, T400S, D401G, H403R, H407W, and A418I;
k) A33V, E65Q, H83G, N90D, K99Q, S100R, K103A, I107K, V108L, N124T, K144A, L184A, K188A, I199M, L207N, I210M, K219T, L227V, T234G, D252R, H280E, S284C, K306R, T310N, K348S, D350G, L390M, G393T, N398T, T400S, D401G, H407W, and A418V;
l) A33V, E65Q, S100R, K103E, N124T, K144A, K188A, I199M, L207N, L227S, D252R, S284C, K306R, T310N, K348S, G393T, N398T, T400S, D401G, H407W, and A418V;
m) S100R, K103E, N124T, K144A, L207N, L227S, D252R, K306R, K348S, N398T, T400S, D401G, H407W, and A418V;
n) A33V, R36S, A39T, K41H, V43T, N90E, K99Q, S100R, I102Q, K103A, H104L, I107T, S117A, N124S, S130D, A140Q, K144Q, F152Y, K154Q, W177C, I183F, L184R, M187L, K188A, N195S, A196C, L207N, I210M, P218A, K219T, L227V, T234G, N237S, G248H, D252R, Y260H, S278A, S284C, D289N, T310N, Y316A, K348S, G349S, D350G, K360N, G364Q, N366T, N368T, S375T, S377T, S380T, I383M, L390M, G393T, N396D, N398T, T400S, D401G, H403R, H407W, and A418I;
o) A33V, V43T, K99Q, S100R, I102Q, K103A, H104R, I107T, N124S, S130N, A140Q, K144A, D148S, F152Y, W177C, L184R, M187I, K188A, A196C, L207N, I210M, K219T, L227V, T234G, Q235S, G248H, D252N, S284C, D289N, T310N, Y316A, K348S, D350G, K360N, G364A, S375T, S377T, L390M, G393T, N398T, T400S, D401G, H403R, H407N, and A418I;
p) K99Q, S100R, I102A, A140Q, T151E, W177C, L207N, L227V, D252R, S284C, K348S, K360N, L390M, N398T, T400S, D401G, H407W, and A418V;
q) S100R, K103E, N124T, K144A, I199L, L207N, L227V, D252R, K306R, K348S, N368Q, N398T, T400S, D401G, H407W, and A418V;
r) K99Q, S100R, I102A, K103A, L207N, L227V, D252E, S284C, K348T, K360N, L390M, G393T, N398T, T400S, D401G, H407W, and A418V;
s) A33V, R64M, E65Q, N90D, K99Q, S100R, K103A, H104N, I107K, V108L, V120I, N124T, K144A, F152Y, L184A, K188A, A196C, I199M, L207N, I210M, K219T, L227V, T234G, G248H, D252R, S278A, H280E, S284C, K306R, T310N, K348S, D350G, L390M, G393T, N398T, T400S, D401G, H407W, and A418V;
t) A33V, V43T, A61P, F68I, H83G, K99Q, S100R, I102Q, K103A, H104N, I107T, N124S, S130N, F152Y, M187I, K188A, A196C, L207N, I210M, K219T, L227V, T234G, G248H, D252N, S284C, D289N, Y316A, K348S, K360N, G364T, S375T, S377T, L390M, G393T, N398T, T400S, D401G, H407Y, and A418I;
u) N398T, T400S, D401G, and A418V;
v) A33V, V43T, H83G, K99Q, S100R, I102Q, K103A, H104N, I107T, N124S, S130N, A140Q, F152Y, W177C, M187I, K188A, A196C, L207N, I210M, K219T, L227V, T234G, Q235S, G248H, D252N, S284C, D289N, Y316A, K348S, K360N, G364Q, N368T, S375T, S377T, L390M, G393T, N398T, T400S, D401G, H407Y, and A418I;
w) A33V, E65Q, S100R, K103E, N124T, K144A, K188A, I199M, L207N, L227V, D252R, S284C, K306R, T310N, K348S, G393T, N398T, T400S, D401G, H407W, and A418V;
x) A33V, R64M, E65Q, H83G, N90D, K99Q, S100R, K103A, H104N, I107K, V102I, N124T, K144A, F152Y, L184A, K188A, A196C, I199M, L207N, I210M, K219T, L227V, T234G, G248H, D252R, S278A, H280E, S284C, K306R, T310N, K348S, D350G, L390M, G393T, N398T, T400S, D401G, H407W, and A418V;
y) A33V, V43T, K99Q, S100R, I102Q, K103A, H104R, I107T, N124S, S130N, A140Q, K144A, D148S, F152Y, W177C, L184R, M187I, K188A, A196C, L207N, I210M, K219T, L227V, T234G, G248H, D252N, S284C, D289N, T310N, Y316A, K348S, D350G, K360N, G364A, S375T, S377T, L390M, G393T, N398T, T400S, D401G, H403R, H407W, and A418I;
z) S100R, K103E, N124T, K144A, I199L, L207N, L227V, D252R, K306R, K348S, N398T, T400S, D401G, H407W, and A418V; or
aa) A33V, R36A, R64M, E65Q, E86P, N90D, K99Q, S100R, K103A, H104R, I107K, V108L, V102I, N124T, S130D, K144A, H145W, F152Y, L184A, M187I, K188A, A196C, I199M, L207N, I210M, K219T, L227V, T234G, N237S, G248H, D252R, S278A, H280E, S284C, D289N, K306R, T310N, K348S, D350G, V353I, N368T, L390M, G393T, N396D, N398T, T400S, D401G, H403Y, H407W, and A418V.

The present invention also provides a xylanase variant of a wild-type xylanase from Actinomadura sp of SEQ ID NO: 32 comprising one or more mutations selected from F55Y, S62G, S79T, T83N, A88C, G96S, R97G, V100I, A104G, T115A, V130I, S132N, W136Y, T145S, I146V, Q157Y, S191A, R200K, Y201H, Y201L, N204H, S207T, M211Q, Y223S, and V228L.

The present invention also provides a xylanase variant of a wild-type xylanase from Actinomadura sp of SEQ ID NO: 32 comprising mutations:
a) F55Y, S62G, S79T, T83N, A88C, G96S, R97G, V100I, A104G, T115A, V130I, S132N, W136Y, T145S, I146V, Q157Y, S191A, R200K, Y201H, S207T, M211Q, Y223S, and V228L;
b) G96S, R97G, A104G, W136Y, T145S, I146V, Q157Y, S191A, Y201H, S207T, Y223S, and V228L;
c) R97G, T145S, Q157Y, S191A, Y201H, S207T, Y223S, and V228L;
d) F55Y, S62G, S79T, T83N, A88C, R97G, V100I, A104G, T115A, V130I, S132N, W136Y, T145S, I146V, Q157Y, S191A, R200K, Y201L, S207T, M211Q, Y223S, and V228L;
e) R97G, W136Y, T145S, Q157Y, S191A, Y201H, S207T, Y223S, and V228L;
f) F55Y, G96S, R97G, A104G, T115A, W136Y, T145S, I146V, Q157Y, S191A, Y201H, S207T, Y223S, and V228L; or
g) S191A, Y201H, N204H, S207T, Y223S, and V228L.

The present invention further provides a xylanase variant of a wild-type xylanase from Aspergillus niger of SEQ ID NO: 33 comprising one or more mutations selected from V29T, V29A, D32H, D32N, K34Q, K34E, F35A, F35M, K36A, K36Q, A37R, A37Q, A37K, T45S, N83S, F98L, S101K, S101K, S101Q, S101T, G103N, L111T, V112L, G128T, G128A, G128K, Q132R, Q132S, Q143G, V149C, V149C, I158A, R180E, V189A, G212A, N215A, H216K, H216N, K218D, V221L, V221I, V225I, A238S, A240Q, N249Q, L251F, G253S, G255N, T256A, K257S, A269G, A269G, V277I, K279Q, K279E, L282C, V288C, V295I, S306E, S307T, A319P, T322N, I324C, I324A, A325V, and A325L.

The present invention further provides a xylanase variant of a wild-type xylanase from Aspergillus niger of SEQ ID NO: 33 comprising mutations:
a) V29T, D32H, F35A, K36A, A37R, T45S, N83S, S101K, G103N, L111T, V112L, G128T, Q132R, G212A, N215A, H216K, V221L, A238S, A240Q, N249Q, G253S, A269G, K279Q, L282C, V288C, S306E, S307T, A319P, T322N, I324C, and A325V;
b) V29T, K36A, A37Q, T45S, S101K, G103N, G128T, Q143G, V149C, R180E, N215A, H216N, V225I, A240Q, G253S, G255N, K257S, A269G, L282C, V288C, S306E, S307T, A319P, T322N, and I324A;
c) V29T, K34Q, F98L, S101Q, G103N, G128A, Q143G, R180E, V189A, N215A, H216K, A240Q, N249Q, G253S, K257S, A269G, K279E, L282C, V288C, S307T, A319P, T322N, and I324A;
d) V29T, G128A, Q143G, R180E, K257S, A269G, K279E, L282C, V288C, S307T, A319P, T322N, and I324A;
e) G128K, Q143G, K257S, A269G, K279E, L282C, V288C, S307T, A319P, and I324A;
f) V29T, D32H, K36A, A37R, T45S, S101K, G103N, L111T, G128T, Q132R, N215A, H216K, V221L, A240Q, G253S, A269G, K279Q, L282C, V288C, S307T, A319P, T322N, I324C, and A325V;
g) V29T, D32H, F35M, K36A, A37R, T45S, N83S, S101K, G103N, L111T, G128A, Q132R, N215A, H216K, V221L, A240Q, N249Q, G253S, A269G, K279Q, L282C, V288C, V295I, S307T, A319P, T322N, I324C, and A325V;
h) D32N, K36A, L111T, G128T, Q132R, G253S, A269G, K279Q, L282C, V288C, S307T, A319P, and I324C;
i) V29T, K34Q, F98L, S101Q, G103N, G128A, Q143G, R180E, V189A, N215A, H216N, A240Q, N249Q, G253S, K257S, A269G, K279E, L282C, V288C, S307T, A319P, T322N, and I324A;
j) V29T, K34Q, K36Q, F98L, S101Q, G103N, G128A, Q143G, I158A, R180E, V189A, G212A, N215A, H216K, V221I, V225I, A238S, A240Q, N249Q, G253S, K257S, A269G, V277I, L282C, V288C, S307T, A319P, T322N, and I324A;
k) V29T, F98L, S101Q, G103N, G128A, Q143G, R180E, V189A, N215A, H216N, A240Q, G253S, K257S, A269G, K279E, L282C, V288C, S307T, A319P, T322N, and I324A;
l) G128T, Q143G, G255N, K257S, A269G, L282C, V288C, S307T, A319P, and I324A;
m) V29T, K34Q, K36A, A37K, T45S, S101K, G103N, G128A, Q132S, Q143G, V149C, R180E, G212A, N215A, H216K, K218D, V221L, V225I, A238S, A240Q, N249Q, G253S, G255N, T256A, K257S, A269G, V277I, L282C, V288C, S306E, S307T, A319P, T322N, and I324A;
n) V29T, D32H, F35M, K36A, T45S, N83S, S101K, G103N, L111T, G128T, Q132R, N215A, H216K, V221L, A240Q, N249Q, G253S, A269G, K279Q, L282C, V288C, S307T, A319P, T322N, I324C, and A325V;
o) S101T, G128T, Q143G, G253S, G255N, K257S, A269G, L282C, V288C, S307T, A319P, and I324A;
p) V29T, K34Q, K36A, A37Q, T45S, S101K, G103N, G128T, Q143G, V149C, R180E, N215A, H216N, V225I, A240Q, L251F, G253S, G255N, K257S, A269G, L282C, V288C, S306E, S307T, A319P, T322N, and I324A;
q) V29T, K34E, F35M, K36A, A37K, T45S, S101K, G103N, G128K, Q132S, Q143G, V149C, R180E, G212A, N215A, H216K, K218D, V221L, V225I, A238S, A240Q, N249Q, G253S, G255N, T256A, K257S, A269G, V277I, L282C, V288C, S306E, S307T, A319P, T322N, I324A, and A325L;
r) V29A, D32H, K36A, A37R, T45S, S101K, G103N, L111T, G128T, Q132R, N215A, H216K, V221L, A240Q, G253S, A269G, K279Q, L282C, V288C, S307T, A319P, T322N, I324C, and A325V;
s) D32H, K36A, L111T, G128T, Q132R, G253S, A269G, K279Q, L282C, V288C, S307T, and A319P;
t) D32H, K36A, L111T, G128T, G253S, A269G, K279Q, L282C, V288C, S307T, and A319P;
u) G128A, Q143G, K257S, A269G, K279E, L282C, V288C, S307T, A319P, and I324A;
v) V29T, K34E, K36Q, F98L, S101Q, G103N, G128K, Q143G, I158A, R180E, V189A, G212A, N215A, H216N, V221I, V225I, A238S, A240Q, N249Q, G253S, K257S, A269G, V277I, L282C, V288C, S307T, A319P, T322N, and I324A;
w) G128A, Q143G, K257S, A269G, L282C, V288C, S307T, and A319P;
x) V29T, G103N, G128T, Q143G, R180E, V225I, G253S, G255N, K257S, A269G, K279Q, L282C, V288C, S307T, A319P, T322N, and I324A;
y) S101T, G128T, Q143G, G255N, K257S, A269G, L282C, V288C, S307T, A319P, and I324A;
z) V29T, S101T, G103N, G128T, Q143G, R180E, V225I, G253S, G255N, K257S, A269G, K279Q, L282C, V288C, S307T, A319P, T322N, and I324A;
aa) V29T, D32N, K36A, T45S, G103N, L111T, G128T, Q132R, N215A, A240Q, G253S, A269G, K279Q, L282C, V288C, S307T, A319P, T322N, and I324C;
bb) V29T, T45S, S101K, G103N, G128T, Q143G, R180E, V225I, A240Q, G253S, G255N, K257S, A269G, K279E, L282C, V288C, S306E, S307T, A319P, T322N, and I324A;
cc) V29T, D32H, K34Q, F35M, K36A, A37R, T45S, N83S, S101K, G103N, L111T, V112L, G128T, Q132R, G212A, N215A, H216K, V221L, A238S, A240Q, N249Q, G253S, A269G, K279Q, L282C, V288C, S307T, A319P, T322N, I324C, and A325V;
dd) V29T, F98L, S101Q, G103N, G128K, Q143G, R180E, V189A, N215A, H216N, A240Q, G253S, K257S, A269G, K279E, L282C, V288C, S307T, A319P, T322N, and I324A; or
ee) L282C, V288C, S307T, and A319P.

The present invention provides a xylanase variant of a wild-type xylanase from Trichoderma reesi of SEQ ID NO: 34 comprising one or more mutations selected from S53G, Q65N, S67D, S67C, N78T, D83Q, T92Q, T92S, A97R, S104Q, N108E, E163Q, N180Q, H203N, L204M, M207H, and S226T.

The present invention provides a xylanase variant of a wild-type xylanase from Trichoderma reesi of SEQ ID NO: 34 comprising mutations:
a) S53G, S67D, D83Q, T92Q, A97R, S104Q, N108E, E163Q, N180Q, H203N, L204M, and S226T;
b) S53G, Q65N, S67C, N78T, D83Q, T92S, A97R, S104Q, N108E, E163Q, N180Q, H203N, L204M, M207H, and S226T;
c) S53G, S67D, A97R, S104Q, N108E, E163Q, H203N, and S226T;
d) S53G, S67D, A97R, S104Q, N108E, E163Q, N180Q, H203N, and S226T;
e) S53G, Q65N, S67D, D83Q, T92S, A97R, S104Q, N108E, E163Q, N180Q, H203N, L204M, and S226T;
f) A97R, and E163Q;
g) S53G, Q65N, S67C, N78T, D83Q, T92Q, A97R, S104Q, N108E, E163Q, N180Q, H203N, L204M, M207H, and S226T;
h) S53G, A97R, and E163Q; or
i) S53G, Q65N, S67C, D83Q, T92S, A97R, S104Q, N108E, E163Q, N180Q, H203N, L204M, and S226T.

The present invention provides a glucanase variant of a wild-type glucanase from Bacillus megaterium of SEQ ID NO: 19 comprising one or more mutations selected from I44V, K45C, T47N, K55N, P56K, P56A, I62V, I62M, L67H, L67T, Y73C, V74Y, D77A, K80D, K80M, L82F, R83A, D84H, G87N, I88A, I88C, F91M, F91M, I103F, I103M, I103F, N110A, K113T, K120L, K120T, L122R, I124M, I128V, N134T, N141H, K150T, S154A, S154N, L155K, S158N, S159T, A167C, D173G, K177A, K177Q, K181R, S189D, S189A, S189N, D195G, T202V, T202C, T206D, N212D, D213S, D213K, D213E, D213E, D216A, D216K, D217N, K220N, A222S, A222D, A227T, G236K, Q237S, Q237D, F238W, D244S, D244R, D244Q, S248Q, S248K, S248N, I253V, G259A, D262S, D262T, Y270N, Q273E, E276R, N279D, Y280W, N290S, N290G, S300D, P315R, T324K, T324I, V326Y, N329A, N329Q, and R331L.

The present invention provides a glucanase variant of a wild-type glucanase from Bacillus megaterium of SEQ ID NO: 19 comprising mutations:
a) I44V, T47N, K55N, P56K, L67H, Y73C, D77A, K80D, I88A, N110A, L122R, I124M, I128V, N141H, S154A, L155K, S158N, S159T, A167C, D173G, S189D, D195G, T202V, D216A, D217N, K220N, A222S, A227T, G236K, Q237S, F238W, D244S, S248Q, I253V, Q273E, E276R, N279D, Y280W, P315R, T324K, V326Y, N329A, and R331L;
b) I44V, K55N, P56K, Y73C, D77A, K80D, I88A, I103F, N110A, I124M, N134T, N141H, S154N, L155K, S158N, S159T, D173G, S189A, D195G, T202V, D216A, D217N, K220N, A222D, A227T, G236K, Q237S, F238W, D244S, S248Q, I253V, Q273E, E276R, N279D, Y280W, P315R, T324K, and R331L;
c) K80M, I88A, I124M, D173G, S189D, D195G, T202V, A227T, D244S, S248K, Y280W, P315R, T324K, and R331L;
d) I44V, P56A, Y73C, D77A, K80D, I88A, I103F, N110A, K120L, I124M, S154N, L155K, S159T, D173G, S189D, D195G, T202V, D216A, D217N, K220N, A222D, A227T, F238W, D244S, S248K, Q273E, Y280W, P315R, T324K, and R331L;
e) I44V, I62V, Y73C, V74Y, D77A, K80D, L82F, I88A, F91M, K113T, I124M, L155K, S158N, S189D, D195G, T202C, D213S, D216A, D217N, K220N, A227T, F238W, D244R, D262S, Q273E, Y280W, N290S, S300D, P315R, T324K, V326Y, and N329Q;
f) I44V, I62V, V74Y, D77A, K80D, L82F, I88C, F91M, K113T, I124M, L155K, S158N, S189N, D195G, T202C, D216A, D217N, K220N, A227T, F238W, D244R, D262T, Y280W, N290S, P315R, T324K, V326Y, and N329Q;
g) I44V, Y73C, K80D, I88A, I124M, L155K, D173G, D195G, T202V, D216A, D217N, K220N, A222D, A227T, F238W, D244S, S248K, Q273E, Y280W, P315R, T324I, and R331L;
h) I44V, P56A, Y73C, D77A, K80D, I88A, N110A, I124M, S154N, L155K, S159T, D173G, S189D, D195G, T202V, D216A, D217N, K220N, A222D, A227T, F238W, D244S, S248K, Q273E, Y280W, P315R, T324K, and R331L;
i) I44V, K80D, I88A I103F, I124M, N134T, S154N, L155K, D173G, D195G, T202V, K220N, A222D, A227T, D244S, S248Q, Y280W, P315R, T324K, and R331L;
j) I62V, V74Y, K80D, L82F, I88C, F91M, K113T, I124M, S189N, D195G, T202C, K220N, A227T, D244R, D262T, Y280W, P315R, T324K, and V326Y;
k) P315R, and R331L;
l) I44V, I62V, V74Y, D77A, K80D, L82F, I88C, F91M, K113T, I124M, L155K, S158N, D195G, T202C, D216A, D217N, K220N, A227T, F238W, D244R, D262T, Y280W, N290S, P315R, T324K, and V326Y;
m) I44V, T47N, K55N, P56K, Y73C, D77A, K80D, I88A, I103F, N110A, L122R, I124M, I128V, N134T, N141H, S154A, L155K, S158N, S159T, A167C, D173G, S189A, D195G, T202V, D216A, D217N, K220N, A222D, A227T, G236K, Q237S, F238W, D244S, S248Q, I253V, Q273E, E276R, N279D, Y280W, P315R, T324K, V326Y, N329A, and R331L;
n) K80M, I88A, I103F, I124M, D173G, S189D, D195G, T202V, A227T, D244S, S248K, Y280W, P315R, T324K, and R331L;
o) I44V, K55N, P56K, Y73C, D77A, K80D, I88A, I103F, N110A, I124M, N141H, L155K, S158N, S159T, D173G, S189A, D195G, T202V, D216A, D217N, K220N, A222D, A227T, G236K, Q237S, F238W, D244S, S248Q, I253V, Q273E, E276R, N279D, Y280W, P315R, T324K, and R331L;
p) I44V, K45C, K55N, I62M, L67T, Y73C, V74Y, D77A, K80D, L82F, R83A, I88A, F91M, I103M, N110A, K113T, I124M, K150T, L155K, S158N, S159T, S189N, D195G, T202C, D213S, D216A, D217N, K220N, A222D, A227T, F238W, D244R, S248N, D262T, Y270N, Q273E, Y280W, N290G, S300D, P315R, T324K, V326Y, and N329Q;
q) I44V, K80D, I88A, I124M, L155K, D173G, D195G, T202V, K220N, A222D, A227T, D244S, S248K, Y280W, P315R, T324K, and R331L;
r) I44V, Y73C, K80D, I88A, I103F, I124M, S154N, L155K, D173G, D195G, T202V, D216K, K220N, A222D, A227T, F238W, D244S, S248K, Q273E, Y280W, P315R, T324K, and R331L;
s) I44V, K45C, K55N, I62M, L67T, Y73C, V74Y, D77A, K80D, L82F, R83A, I88A, F91M, I103M, N110A, K113T, I124M, K150T, L155K, S158N, S159T, S189D, D195G, T202C, D213K, D216A, D217N, K220N, A222D, A227T, F238W, D244Q, S248N, D262S, Y270N, Q273E, Y280W, N290G, P315R, T324K, V326Y, and N329Q;
t) I62V, V74Y, K80D, L82F, I88A, F91M, K113T, I124M, D195G, T202C, K220N, A227T, D244R, D262T, Y280W, N290S, P315R, T324K, V326Y, and N329Q;
u) I44V, K45C, T47N, K55N, I62M, L67T, Y73C, V74Y, D77A, K80D, L82F, R83A, D84H, G87N, I88A, F91M, I103M, N110A, K113T, K120T, L122R, I124M, N141H, K150T, L155K, S158N, S159T, K177A, K181R, S189D, D195G, T202C, T206D, N212D, D213E, D216A, D217N, K220N, A222S, A227T, Q237D, F238W, D244R, S248N, I253V, G259A, D262S, Y270N, Q273E, E276R, Y280W, N290G, P315R, T324K, V326Y, and N329Q;
v) I44V, I62V, Y73C, V74Y, D77A, K80D, L82F, I88A, F91M, K113T, I124M, L155K, S158N, S189D, D195G, T202C, D213S, D216A, D217N, K220N, A227T, F238W, D244R, D262T, Q273E, Y280W, N290S, S300D, P315R, T324K, V326Y, and N329Q;
w) I62V, V74Y, K80D, L82F, I88A, F91M, K113T, I124M, S189N, D195G, T202C, K220N, A227T, D244R, D262T, Y280W, N290S, P315R, T324K, and V326Y;
x) I44V, I62V, Y73C, V74Y, D77A, K80D, L82F, I88A, F91M, K113T, I124M, L155K, S158N, S189D, D195G, T202C, D213E, D216A, D217N, K220N, A222D, A227T, F238W, D244Q, S248N, D262S, Y270N, Q273E, Y280W, N290S, P315R, T324K, V326Y, and N329Q;
y) I44V, I62V, Y73C, V74Y, D77A, K80D, L82F, I88C, F91M, K113T, I124M, L155K, S158N, K177Q, D195G, T202C, D213S, D216A, D217N, K220N, A222D, A227T, F238W, D244R, S248N, G259A, D262S, Y270N, Q273E, Y280W, N290S, P315R, T324K, V326Y, and N329Q; or
z) I44V, K45C, T47N, K55N, I62M, L67T, Y73C, V74Y, D77A, K80D, L82F, R83A, D84H, G87N, I88A, F91M, I103M, N110A, K113T, K120T, L122R, I124M, N141H, K150T, L155K, S158N, S159T, K177A, K181R, S189D, D195G, T202C, T206D, N212D, D213E, D216A, D217N, K220N, A222S, A227T, Q237D, F238W, D244R, S248N, I253V, D262T, Y270N, Q273E, E276R, Y280W, N290G, S300D, P315R, T324K, V326Y, and N329Q.

The present invention provides a glucose isomerase variant of a wild-type glucose isomerase from Streptomyces murinus of SEQ ID NO: 20 comprising one or more mutations selected from F3Y, R10K, T39Y, T39I, Y49W, G62D, T66A, S70K, R76K, Q77D, A81D, T85K, A89V, A106S, T119V, I120M, G121R, I123L, A136F, A136M, A143F, A143T, G146L, D153R, G166A, E167D, A171K, A171E, L176I, D190H, L192Y, V196T, V196A, V196I, E204Y, Y212F, L226H, G231E, A238H, A301E, I312A, D315E, R316K, A318I, A318R, A319K, Q337E, D348T, D348A, A355S, H374K, H374W, and L383T.

The present invention provides a glucose isomerase variant of a wild-type glucose isomerase from Streptomyces murinus of SEQ ID NO: 20 comprising mutations:
a) T39Y, Y49W, T85K, T119V, I120M, G121R, A136F, G146L, D153R, A171K, D190H, E204Y, G231E, A301E, I312A, R316K, D348T, and L383T;
b) F3Y, R10K, T39Y, Y49W, G62D, T66A, S70K, R76K, A81D, T85K, A106S, T119V, I120M, G121R, I123L, A136M, G146L, D153R, G166A, E167D, A171E, D190H, L192Y, V196T, E204Y, Y212F, L226H, G231E, A238H, A301E, I312A, D315E, R316K, A318I, A319K, Q337E, D348A, A355S, H374K, and L383T;
c) T39Y, Y49W, T85K, I120M, G121R, A136F, G146L, D153R, A171K, D190H, E204Y, G231E, A301E, I312A, R316K, D348T, and L383T;
d) F3Y, T39Y, Y49W, A81D, T85K, T119V, I120M, G121R, A136F, G146L, D153R, A171E, D190H, E204Y, Y212F, G231E, A301E, I312A, D315E, R316K, A319K, D348A, A355S, and L383T;
e) T39I, Y49W, T85K, T119V, I120M, G121R, A136F, A171K, D190H, E204Y, G231E, A301E, R316K, D348T, and L383T;
f) F3Y, R10K, T39Y, Y49W, G62D, Q77D, A81D, T85K, T119V, I120M, G121R, I123L, A136F, A143F, D153R, A171E, D190H, Y212F, G231E, A301E, I312A, D315E, R316K, A319K, D348A, A355S, H374W, and L383T;
g) F3Y, T39Y, Y49W, A81D, T85K, T119V, I120M, G121R, A136F, D153R, A171E, D190H, Y212F, G231E, A301E, I312A, D315E, R316K, A319K, D348A, A355S, and L383T;
h) T39Y, Y49W, T85K, T119V, I120M, G121R, A136M, G146L, A171K, D190H, E204Y, G231E, A301E, R316K, D348T, and L383T;
i) F3Y, T39Y, Y49W, T85K, T119V, I120M, G121R, A136F, G146L, D153R, A171E, D190H, E204Y, G231E, A301E, I312A, R316K, D348A, A355S, and L383T;
j) F3Y, R10K, T39Y, Y49W, G62D, T66A, S70K, R76K, A81D, T85K, A106S, I120M, G121R, A136M, A143T, G146L, D153R, G166A, E167D, A171E, L176I, D190H, L192Y, V196T, Y212F, L226H, G231E, A238H, A301E, I312A, R316K, A318R, Q337E, D348A, A355S, H374K, and L383T;
k) F3Y, R10K, T39Y, Y49W, G62D, T66A, S70K, R76K, A81D, T85K, A89V, A106S, T119V, I120M, G121R, I123L, A136F, D153R, G166A, A171E, L176I, D190H, L192Y, V196A, Y212F, G231E, A238H, A301E, I312A, D315E, R316K, A318R, D348A, A355S, H374K, and L383T;
l) F3Y, R10K, T39Y, G62D, T66A, S70K, R76K, A81D, T85K, A89V, A106S, T119V, I120M, G121R, I123L, A136F, D153R, G166A, A171E, L176I, D190H, L192Y, Y212F, G231E, A238H, A301E, I312A, D315E, R316K, A318R, D348A, A355S, H374K, and L383T; or
m) F3Y, R10K, T39Y, G62D, Q77D, A81D, T85K, A89V, T119V, I120M, G121R, I123L, A136F, A143F, D153R, A171E, L176I, D190H, V196I, Y212F, A301E, I312A, D315E, R316K, A319K, D348A, A355S, H374W, and L383T.

The present invention provides an inulase variant of a wild-type inulase from Aspergillus niger of SEQ ID NO: 21 comprising one or more mutations selected S25R, S25T, D37K, Q38A, S52G, T53V, W54Y, F58Y, F58Y, T63Y, N69P, I70M, M81L, A84D, A84E, T88V, A91P, D92S, T100S, T102S, D115T, A117T, A117D, A117K, T129N, T130K, S132Q, Q133G, F141Y, V143T, T150V, R183E, R183E, Q184E, N187R, N187K, N187Q, G196S, D198H, D198N, A206T, A206D, T220S, S221Q, E246N, A281P, A286P, N293Y, V304E, T329N, S342Q, V346I, Q350R, H351R, T358S, S364G, A371Q, A371T, A395K, A395E, A400P, A412G, T421R, A425N, K450Q, K450T, E452S, E453P, G455S, S460H, Q476D, D489P, D489E, N502S, N502S, N502G, S507C, S512Y, S512Y, and V513.

The present invention provides an inulase variant of a wild-type inulase from Aspergillus niger of SEQ ID NO: 21 comprising mutations:
a) Q38A, S52G, I70M, A84D, A91P, T100S, R183E, D198H, N293Y, Q350R, H351R, A400P, S460H, D489P, and N502S;
b) S25R, Q38A, S52G, I70M, A84D, A91P, T100S, S132Q, Q133G, R183E, N187R, D198H, A206T, N293Y, V304E, V346I, Q350R, H351R, A400P, E453P, S460H, D489P, and N502S;
c) S25R, D37K, Q38A, S52G, N69P, I70M, A84E, A91P, D92S, T100S, D115T, A117T, S132Q, Q133G, V143T, R183E, N187K, D198N, A206D, S221Q, E246N, A286P, N293Y, V304E, V346I, Q350R, H351R, T358S, A371Q, A395K, A400P, K450Q, E453P, G455S, S460H, Q476D, D489E, N502S, S507C, and S512Y;
d) Q38A, S52G, I70M, A84D, A91P, T100S, R183E, D198H, A281P, N293Y, V346I, Q350R, H351R, A400P, S460H, and N502S;
e) S25R, D37K, Q38A, S52G, W54Y, T63Y, I70M, M81L, A84D, A91P, D92S, T100S, D115T, A117D, T129N, S132Q, Q133G, V143T, R183E, Q184E, N187K, D198N, A206T, S221Q, E246N, A286P, N293Y, V304E, T329N, V346I, Q350R, H351R, T358S, A371T, A395E, A400P, A412G, A425N, K450T, E453P, G455S, S460H, Q476D, D489E, N502S, S507C, and S512Y;
f) S25R, Q38A, S52G, I70M, A84D, A91P, T100S, S132Q, Q133G, R183E, N187R, D198H, A206T, A281P, N293Y, V304E, V346I, Q350R, H351R, A400P, E453P, S460H, D489P, and N502S;
g) S25R, D37K, Q38A, S52G, T63Y, I70M, A84E, A91P, D92S, T100S, D115T, A117T, S132Q, Q133G, V143T, R183E, N187K, D198N, A206T, S221Q, E246N, A286P, N293Y, V304E, V346I, Q350R, H351R, T358S, A371Q, A395K, A400P, K450Q, E453P, G455S, S460H, Q476D, D489E, N502G, S507C, and S512Y;
h) S25T, D37K, Q38A, S52G, W54Y, T63Y, I70M, A84E, A91P, D92S, T100S, D115T, A117K, T129N, S132Q, Q133G, V143T, R183E, Q184E, N187K, D198N, A206T, S221Q, E246N, A286P, N293Y, V304E, T329N, Q350R, H351R, T358S, S364G, A371T, A395K, A400P, A412G, A425N, K450T, E453P, G455S, S460H, Q476D, D489E, N502S, S507C, S512Y, and V513I;
i) S25R, D37K, Q38A, S52G, T63Y, I70M, A84D, A91P, D92S, T100S, D115T, A117T, S132Q, Q133G, R183E, N187K, D198H, A206D, S221Q, E246N, A286P, N293Y, V304E, V346I, Q350R, H351R, A371Q, A395K, A400P, E453P, S460H, D489E, N502S, S507C, and S512Y;
j) S25R, D37K, Q38A, S52G, N69P, I70M, A84D, A91P, T100S, D115T, A117T, S132Q, Q133G, R183E, N187Q, D198H, A206T, S221Q, A286P, N293Y, V304E, V346I, Q350R, H351R, A400P, E453P, S460H, D489E, N502S, and S512Y;
k) S25R, D37K, Q38A, S52G, T63Y, I70M, A84D, A91P, D92S, T100S, D115T, A117T, S132Q, Q133G, R183E, N187K, D198H, A206T, S221Q, E246N, A286P, N293Y, V304E, V346I, Q350R, H351R, A371Q, A395K, A400P, E453P, S460H, D489E, N502S, S507C, and S512Y;
l) S25R, D37K, Q38A, S52G, T53V, W54Y, F58Y, T63Y, I70M, A84E, T88V, A91P, D92S, T100S, T102S, D115T, A117K, T129N, T130K, S132Q, Q133G, F141Y, V143T, T150V, R183E, Q184E, N187K, G196S, D198N, A206D, T220S, S221Q, E246N, A286P, N293Y, V304E, T329N, S342Q, V346I, Q350R, H351R, T358S, A371T, A395K, A400P, A412G, T421R, A425N, K450T, E452S, E453P, G455S, S460H, Q476D, D489E, N502S, S507C, and S512Y;
m) S25R, D37K, Q38A, S52G, N69P, I70M, A84D, A91P, T100S, D115T, A117T, S132Q, Q133G, R183E, N187Q, D198H, A206T, S221Q, A286P, N293Y, V304E, Q350R, H351R, A400P, E453P, S460H, D489E, N502S, and S512Y; or
n) S25R, D37K, Q38A, S52G, T53V, W54Y, F58Y, T63Y, I70M, M81L, A84D, T88V, A91P, D92S, T100S, T102S, D115T, A117K, T129N, T130K, S132Q, Q133G, F141Y, V143T, T150V, R183E, Q184E, N187K, G196S, D198N, A206T, T220S, S221Q, E246N, A286P, N293Y, V304E, T329N, S342Q, V346I, Q350R, H351R, T358S, A371T, A395K, A400P, A412G, T421R, A425N, K450T, E452S, E453P, G455S, S460H, Q476D, D489E, N502G, S507C, and S512Y.

The present invention provides a lactase variant of a wild-type lactase from Kluyveromyces of SEQ ID NO: 22 comprising one or more mutations selected from N36S, N36G, F41C, A42G, A50V, K62S, V69A, S71G, E74S, K80S, N88S, T112S, E114S, D116P, K118D, I120S, I120K, E121D, N134S, L138V, N148A, K149Q, I159V, E167D, K173R, F175Y, K176Q, S180G, K202A, T216V, S278C, K308H, V371I, K379Q, F380H, N387C, N387T, S388A, F400C, I408M, T415C, N423A, K438L, D441K, V442E, H445K, Y446W, V455P, V455K, V455E, S462E, L464M, V469K, C484G, N512H, L514E, L514E, F526V, F526I, H536E, S562G, E573Y, K574P, K574D, Y576L, I581V, V701S, E768Q, L770M, S779T, I816V, V832I, T833I, N835R, S839A, V841A, V842G, Y844A, Y844A, K852T, A857G, N861K, N863R, S865T, G887S, Y916F, E946Q, E957D, and E999A.

The present invention provides a lactase variant of a wild-type lactase from Kluyveromyces of SEQ ID NO: 22 comprising mutations:
a) N36S, A42G, K62S, V69A, K80S, N88S, T112S, E114S, D116P, K118D, I120S, N134S, L138V, K149Q, E167D, S180G, K202A, S278C, K308H, V371I, K379Q, F380H, N387C, S388A, F400C, I408M, T415C, V455P, S462E, L464M, V469K, C484G, L514E, S562G, E573Y, K574P, Y576L, L770M, N835R, S839A, V842G, Y844A, K852T, A857G, G887S, E946Q, E957D, and E999A;
b) N36S, A42G, K62S, V69A, K80S, N88S, T112S, E114S, D116P, K118D, I120S, N134S, L138V, K149Q, E167D, S180G, K202A, S278C, K308H, V371I, K379Q, F380H, N387C, S388A, F400C, I408M, T415C, V455P, S462E, L464M, V469K, C484G, L514E, S562G, E573Y, K574P, Y576L, N835R, S839A, V842G, Y844A, K852T, A857G, G887S, E946Q, E957D, and E999A;
c) N36S, F41C, A42G, K62S, V69A, S71G, T112S, E114S, D116P, I120K, N134S, F175Y, S180G, K202A, V371I, K379Q, N387C, T415C, V455K, S462E, V469K, C484G, L514E, S562G, E573Y, Y576L, L770M, N835R, V842G, Y844A, G887S, and E957D;
d) N36S, A42G, K62S, V69A, K80S, N88S, T112S, E114S, D116P, K118D, I120S, N134S, L138V, K149Q, E167D, F175Y, K176Q, S180G, K202A, S278C, K308H, V371I, K379Q, F380H, N387C, S388A, F400C, I408M, T415C, D441K, Y446W, V455P, S462E, L464M, V469K, C484G, L514E, F526V, S562G, E573Y, K574D, Y576L, I581V, E768Q, S779T, N835R, S839A, V842G, Y844A, K852T, A857G, N861K, G887S, Y916F, E946Q, E957D, and E999A;
e) N36S, F41C, A42G, K62S, V69A, E74S, K80S, N88S, T112S, E114S, D116P, K118D, I120S, E121D, N134S, L138V, N148A, K149Q, I159V, E167D, K173R, F175Y, K176Q, S180G, K202A, T216V, S278C, K308H, V371I, K379Q, F380H, N387C, F400C, I408M, T415C, N423A, K438L, D441K, V442E, H445K, Y446W, V455P, S462E, L464M, V469K, C484G, N512H, L514E, F526I, H536E, S562G, E573Y, K574D, Y576L, V701S, E768Q, S779T, I816V, V832I, T833I, N835R, S839A, V841A, V842G, Y844A, K852T, A857G, N861K, N863R, S865T, G887S, Y916F, E946Q, E957D, and E999A;
f) N36S, F41C, A42G, A50V, K62S, V69A, E74S, K80S, N88S, T112S, E114S, D116P, K118D, I120S, E121D, N134S, L138V, N148A, K149Q, I159V, E167D, K173R, F175Y, S180G, K202A, T216V, S278C, K308H, V371I, K379Q, F380H, N387T, S388A, F400C, I408M, T415C, N423A, K438L, D441K, V442E, H445K, Y446W, V455P, S462E, L464M, V469K, C484G, L514E, F526I, H536E, S562G, E573Y, K574D, Y576L, V701S, L770M, S779T, I816V, V832I, T833I, N835R, S839A, V841A, V842G, Y844A, K852T, A857G, N861K, N863R, S865T, G887S, Y916F, E946Q, E957D, and E999A;
g) N36G, F41C, A42G, A50V, K62S, V69A, E74S, K80S, N88S, T112S, E114S, D116P, K118D, I120K, N134S, L138V, N148A, K149Q, E167D, K176Q, S180G, K202A, S278C, K308H, V371I, K379Q, F380H, N387C, S388A, F400C, I408M, T415C, K438L, D441K, H445K, Y446W, V455P, S462E, L464M, V469K, C484G, L514E, F526I, S562G, E573Y, K574D, Y576L, L770M, S779T, T833I, N835R, S839A, V842G, Y844A, K852T, A857G, N861K, S865T, G887S, Y916F, E946Q, E957D, and E999A;
h) N36S, F41C, A42G, K62S, V69A, S71G, N88S, T112S, E114S, D116P, K118D, I120S, N134S, L138V, K149Q, E167D, F175Y, S180G, K202A, S278C, V371I, K379Q, F380H, N387C, S388A, F400C, I408M, T415C, V455P, S462E, L464M, V469K, C484G, L514E, S562G, E573Y, K574P, Y576L, N835R, S839A, V842G, Y844A, A857G, G887S, and E957D;
i) N36S, A42G, K62S, V69A, S71G, N88S, T112S, E114S, D116P, K118D, I120S, N134S, K149Q, E167D, F175Y, S180G, K202A, S278C, V371I, K379Q, F380H, N387C, S388A, F400C, I408M, T415C, V455P, S462E, L464M, V469K, C484G, L514E, S562G, E573Y, K574P, Y576L, L770M, N835R, S839A, V842G, Y844A, A857G, G887S, and E957D;
j) N36S, F41C, A42G, A50V, K62S, V69A, S71G, E74S, K80S, N88S, T112S, E114S, D116P, K118D, I120K, N134S, L138V, N148A, K149Q, E167D, K176Q, S180G, K202A, S278C, K308H, V371I, K379Q, F380H, N387C, S388A, F400C, I408M, T415C, K438L, D441K, H445K, Y446W, V455P, S462E, L464M, V469K, C484G, L514E, F526I, S562G, E573Y, K574D, Y576L, E768Q, S779T, T833I, N835R, S839A, V842G, Y844A, K852T, A857G, N861K, S865T, G887S, Y916F, E946Q, E957D, and E999A;
k) N36S, F41C, A42G, K62S, V69A, S71G, T112S, E114S, D116P, I120S, N134S, S180G, K202A, K379Q, N387C, T415C, V455K, S462E, V469K, C484G, L514E, S562G, E573Y, Y576L, N835R, V842G, and G887S;
l) N36G, A42G, K62S, V69A, T112S, E114S, D116P, I120S, N134S, F175Y, S180G, K202A, V371I, K379Q, N387C, T415C, V455E, S462E, V469K, C484G, L514E, S562G, E573Y, Y576L, N835R, V842G, Y844A G887S, and E957D; or
m) N36S, F41C, A42G, A50V, K62S, V69A, K80S, N88S, T112S, E114S, D116P, K118D, I120S, N134S, L138V, K149Q, E167D, K176Q, S180G, K202A, S278C, K308H, V371I, K379Q, F380H, N387T, S388A, F400C, I408M, T415C, D441K, Y446W, V455P, S462E, L464M, V469K, C484G, N512H, L514E, F526V, S562G E573Y, K574D, Y576L, E768Q, S779T, N835R, S839A, V842G, Y844A, K852T, A857G, N861K, G887S, Y916F, E946Q, E957D, and E999A.

The present invention provides a lysozyme variant of a wild-type lysozyme from chicken of SEQ ID NO: 23 comprising one or more mutations selected from F21Y, G22T, A28R, A28K, N37G, R39K, G44A, K51Y, F52Y, F52H, S90A, R91K, S104D, S109A, N111E, V127D, R132H, T136H, T136R, T136K, Q139S, A140K, and R146D.

The present invention provides a lysozyme variant of a wild-type lysozyme from chicken of SEQ ID NO: 23 comprising mutations:
a) G22T, A28R, N37G, G44A, K51Y, F52Y, S109A, N111E, R132H, T136H, Q139S, A140K, and R146D;
b) G22T, A28R, N37G, G44A, K51Y, F52H, S109A, R132H, T136R, Q139S, A140K, and R146D;
c) K51Y, R132H, and Q139S;
d) N37G, G44A, K51Y, F52H, S109A, R132H, and Q139S;
e) G22T, A28R, N37G, R39K, G44A, K51Y, F52Y, S90A, S109A, N111E, V127D, R132H, T136H, Q139S, A140K, and R146D;
f) G44A, K51Y, S109A, R132H, and Q139S;
g) F21Y, A28R, N37G, R39K, G44A, K51Y, F52Y, S90A, R91K, S109A, N111E, V127D, R132H, T136H, Q139S, A140K, and R146D;
h) F21Y, A28R, N37G, R39K, K51Y, F52Y, S90A, R91K, S104D, S109A, N111E, V127D, R132H, T136H, Q139S, A140K, and R146D; or
i) G22T, A28K, N37G, R39K, G44A, K51Y, F52Y, S90A, S109A, N111E, V127D, R132H, T136K, Q139S, A140K, and R146D.

The present invention provides a pectin lyase variant of a wild-type pectin lyase from Aspergillus niger SEQ ID NO: 24 comprising one or more mutations selected from V24S, V24Q, V24T, H32S, D49A, N58S, T77S, A84C, Q91A, Q93C, L98Q, L98K, H99N, H99D, S100N, D103C, D103C, K118N, I121T, T137S, V140A, V148I, K153E, A161H, V162I, I165L, K168Q, V178I, D179N, Y213N, S214N, L215D, I216F, D217Y, R219A, R219N, D221S, N227C, Q229H, Y235M, Y235L, L236F, M242Q, F250I, F250V, N252H, L253T, L253S, M257G, K259A, K259D, Q261G, L272V, F273W, H274Y, N275Q, N275S, F276N, D277N, I283V, A291I, A291L, I307L, Q310H, S313N, S314N, A324C, Q332C, S339N, T361A, N367A, N367M, N367A, N367R, I368Q, Q370E, T372V, T372A, T372V, T372A, T372A, M373L, Q378A, and Q378A.

The present invention provides a pectin lyase variant of a wild-type pectin lyase from Aspergillus niger SEQ ID NO: 24 comprising mutations:
a) H32S, T77S, A84C, Q93C, H99N, S100N, D103C, I121T, T137S, K153E, D179N, L215D, R219A, D221S, N227C, Y235M, M242Q, N252H, L253T, M257G, K259A, Q261G, F276N, I283V, Q310H, A324C, Q332C, T361A, N367A, T372V, and M373L;
b) V24S, H32S, D49A, T77S, A84C, Q91A, Q93C, L98Q, H99N, S100N, D103C, K118N, I121T, T137S, K153E, A161H, K168Q, V178I, D179N, S214N, L215D, R219N, D221S, N227C, Q229H, Y235M, L236F, M242Q, F250I, N252H, L253T, M257G, K259D, Q261G, L272V, F273W, H274Y, F276N, Q310H, S314N, A324C, Q332C, T361A, T372A, and Q378A;
c) V24Q, H32S, T77S, A84C, Q91A, Q93C, L98Q, H99N, S100N, D103C, I121T, T137S, K153E, A161H, K168Q, D179N, L215D, R219A, D221S, N227C, Q229H, Y235M, M242Q, F250I, N252H, L253T, M257G, K259D, Q261G, L272V, F273W, H274Y, F276N, I283V, Q310H, A324C, Q332C, T361A, N367A, T372A, and Q378A;
d) V24S, D49A, T77S, A84C, Q91A, Q93C, L98K, H99N, S100N, D103C, T137S, V140A, V148I, V178I, D179N, Y213N, L215D, I216F, N227C, Q229H, N252H, L253S, M257G, K259A, N275Q, F276N, A291I, I307L, A324C, Q332C, S339N, T361A, N367M, T372V, and Q378A;
e) A84C, Q93C, D103C, N227C, A324C, Q332C, T361A, and N367A;
f) H32S, T77S, A84C, Q93C, H99D, S100N, D103C, T137S, L215D, D217Y, R219A, N227C, Y235L, M242Q, N252H, L253T, M257G, K259A, Q261G, F276N, Q310H, A324C, Q332C, T361A, N367A, and T372V;
g) H32S, T77S, A84C, Q93C, H99D, S100N, D103C, T137S, V140A, L215D, D217Y, R219A, N227C, Y235L, M242Q, N252H, L253T, M257G, Q261G, F276N, Q310H, A324C, Q332C, T361A, N367A, and T372V;
h) H32S, T77S, A84C, Q91A, Q93C, H99N, S100N, D103C, I121T, T137S, V140A, K153E, A161H, D179N, L215D, R219A, D221S, N227C, Q229H, Y235M, M242Q, F250V, N252H, L253T, M257G, K259D, Q261G, L272V, F273W, F276N, I283V, Q310H, A324C, Q332C, T361A, N367A, and T372A;
i) H32S, T77S, A84C, Q93C, H99D, S100N, D103C, I121T, T137S, V140A, L215D, R219A, D221S, N227C, Y235M, M242Q, N252H, L253T, M257G, K259D, Q261G, F276N, Q310H, A324C, Q332C, T361A, N367A, and T372V;
j) T77S, A84C, Q93C, H99N, S100N, D103C, T137S, V140A, D179N, Y213N, N227C, Q229H, N252H, L253S, M257G, F273W, F276N, A324C, Q332C, T361A, and N367A;
k) V24S, H32S, D49A, T77S, A84C, Q91A, Q93C, L98Q, H99N, S100N, D103C, K118N, I121T, T137S, K153E, A161H, K168Q, V178I, D179N, S214N, L215D, R219N, D221S, N227C, Q229H, Y235M, L236F, M242Q, F250I, N252H, L253T, M257G, K259A, Q261G, L272V, F273W, H274Y, F276N, I283V, Q310H, S314N, A324C, Q332C, T361A, T372A, and Q378A;
l) H32S, T77S, A84C, Q93C, H99N, S100N, D103C, T137S, V140A, K153E, D179N, L215D, R219A, D221S, N227C, Y235M, M242Q, N252H, L253T, M257G, K259A, Q261G, F276N, I283V, Q310H, A324C, Q332C, T361A, N367A, T372V, and M373L;
m) D49A, T77S, A84C, Q93C, L98K, H99N, S100N, D103C, T137S, V140A, V148I, D179N, Y213N, L215D, N227C, Q229H, N252H, L253S, M257G, K259A, F273W, F276N, A324C, Q332C, T361A, N367A, and T372V;
n) V24S, H32S, D49A, N58S, T77S, A84C, Q91A, Q93C, L98Q, H99N, S100N, D103C, K118N, I121T, T137S, K153E, A161H, V162I, K168Q, V178I, D179N, Y213N, S214N, L215D, R219N, D221S, N227C, Q229H, Y235M, L236F, M242Q, F250I, N252H, L253T, M257G, K259D, Q261G, L272V, F273W, H274Y, F276N, I283V, A291L, Q310H, S313N, S314N, A324C, Q332C, T361A, T372A, and Q378A;
o) V24S, D49A, T77S, A84C, Q91A, Q93C, L98K, H99N, S100N, D103C, T137S, V140A, V148I, V178I, D179N, Y213N, L215D, N227C, Q229H, N252H, L253S, M257G, K259A, F273W, N275Q, F276N, A291I, I307L, A324C, Q332C, S339N, T361A, N367M, T372V, and Q378A;
p) T77S, A84C, Q93C, H99N, S100N, D103C, T137S, V140A, D179N, Y213N, N227C, N252H, L253S, M257G, F276N, A324C, Q332C, T361A, and N367A;
q) V24Q, H32S, T77S, A84C, Q91A, Q93C, L98Q, H99N, S100N, D103C, I121T, T137S, K153E, A161H, K168Q, D179N, L215D, R219A, D221S, N227C, Q229H, Y235M, M242Q, F250I, N252H, L253T, M257G, K259A, Q261G, L272V, F273W, H274Y, F276N, I283V, Q310H, A324C, Q332C, T361A, N367A, T372A and, Q378A;
r) V24T, D49A, N58S, T77S, A84C, Q91A, Q93C, L98K, H99N, S100N, D103C, T137S, V140A, V148I, A161H, V162I, I165L, V178I, D179N, Y213N, L215D, N227C, Q229H, N252H, L253S, M257G, K259A, N275S, F276N, D277N, A291I, I307L, A324C, Q332C, S339N, T361A, Q370E, T372V, and Q378A;
s) V24S, D49A, N58S, T77S, A84C, Q91A, Q93C, L98K, H99N, S100N, D103C, T137S, V140A, V148I, A161H, V162I, I165L, V178I, D179N, Y213N, L215D, I216F, N227C, Q229H, N252H, L253S, M257G, K259A, F273W, N275Q, F276N, D277N, A291I, I307L, A324C, Q332C, S339N, T361A, N367R, Q370E, T372V, and Q378A;
t) V24S, D49A, T77S, A84C, Q91A, Q93C, L98K, H99N, S100N, D103C, T137S, V140A, V148I, V178I, D179N, Y213N, L215D, N227C, Q229H, N252H, L253S, M257G, K259A, F273W, N275Q, F276N, A324C, Q332C, T361A, N367A, T372V, and Q378A;
u) V24S, H32S, D49A, N58S, T77S, A84C, Q91A, Q93C, L98Q, H99N, S100N, D103C, K118N, I121T, T137S, K153E, A161H, V162I, K168Q, V178I, D179N, Y213N, S214N, L215D, R219N, D221S, N227C, Q229H, Y235M, L236F, M242Q, F250I, N252H, L253T, M257G, K259A, Q261G, L272V, F273W, H274Y, F276N, I283V, A291L, Q310H, S313N, S314N, A324C, Q332C, T361A, T372A, and Q378A;
v) H32S, T77S, A84C, Q93C, H99D, S100N, D103C, I121T, T137S, V140A, L215D, R219A, D221S, N227C, Y235M, M242Q, N252H, L253T, M257G, K259A, Q261G, F276N, Q310H, A324C, Q332C, T361A, N367A, and T372V;
w) H32S, T77S, A84C, Q91A, Q93C, H99N, S100N, D103C, T137S, A161H, D179N, L215D, R219A, D221S, N227C, Q229H, Y235M, M242Q, F250V, N252H, L253T, M257G, K259A, Q261G, L272V, F273W, F276N, I283V, Q310H, A324C, Q332C, T361A, N367A, and T372A; or
x) T77S, A84C, Q93C, H99N, S100N, D103C, T137S, V140A, D179N, Y213N, L215D, N227C, Q229H, N252H, L253S, M257G, K259A, F273W, F276N, A324C, Q332C, T361A, N367A, and I368Q.

The present invention provides a phytase variant of a wild-type phytase from Escherichia coli of SEQ ID NO: 25 comprising one or more mutations selected from P26G, E27D, S37M, A47P, T48P, L50T, D53R, V54M, D57H, D57S, D57R, A58P, T61Q, K65A, L66A, Q84F, L88W, L88M, V89R, V89M, A90Q, D91Q, A95Q, P100C, S102D, S102A, S102P, I108W, D110N, E113Q, G119A, E120A, F122Y, L126A, D129G, A131C, I132V, T133R, T136Y, T140M, T140L, S141G, S142T, S142K, P143M, V154G, Q156C, A160K, A160Q, N161Q, V162A, T163V, D164A, I166V, S168K, S173G, A175Q, D176A, F177E, T178H, G179E, G179A, G179R, H180R, T183A, T183E, F185Y, E187Q, R190K, L201C, Q206C, S234A, L235R, T240A, P252Q, E253D, G255A, T260H, S262E, S262A, H263Q, H263Q, N266A, T267R, L269M, S270E, S270A, Y277H, Q280F, T282A, E284Y, R287A, S288A, T291G, P292Y, D295Q, D295R, L296F, M298Q, A299T, K308N, Q309N, T314A, L315H, L320G, I322M, A335M, E337G, L342F, E354A, E354K, R362D, L363K, S364A, D365A, N366Y, S367K, I370V, S373A, F376Y, M382L, D384Q, K385A, N391A, T392Q, T400C, T400C, A402Q, A416D, G417Q, G417R, and G417Q.

The present invention provides a phytase variant of a wild-type phytase from Escherichia coli of SEQ ID NO: 25 comprising mutations:
a) P26G, E27D, S37M, A47P, T48P, D57H, A58P, T61Q, K65A, L66A, V89R, A90Q, A95Q, P100C, S102D, D110N, E113Q, G119A, E120A, L126A, A131C, T133R, T136Y, T140M, S141G, S142T, P143M, V154G, Q156C, A160K, N161Q, S168K, S173G, A175Q, D176A, F177E, T178H, G179E, H180R, T183A, F185Y, R190K, L201C, Q206C, L235R, T240A, P252Q, E253D, G255A, S262E, H263Q, N266A, T267R, L269M, S270E, Y277H, Q280F, T282A, E284Y, R287A, S288A, T291G, M298Q, K308N, I322M, A335M, E337G, L342F, E354A, R362D, L363K, S364A, N366Y, S367K, I370V, S373A, F376Y, K385A, N391A, T392Q, T400C, and G417Q;
b) P26G, S37M, T48P, K65A, L66A, V89R, A90Q, A95Q, P100C, S102A, D110N, E120A, L126A, A131C, T140L, S141G, S142T, P143M, V154G, Q156C, N161Q, S168K, S173G, A175Q, G179A, T183E, F185Y, R190K, L201C, Q206C, T240A, P252Q, G255A, S262A, H263Q, T267R, Y277H, T282A, E284Y, R287A, S288A, A335M, L342F, E354K, R362D, S364A, N366Y, S367K, F376Y, K385A, T392Q, T400C, and G417Q;
c) P26G, S37M, T48P, K65A, L88W, V89M, A90Q, A95Q, P100C, S102A, D110N, E120A, L126A, A131C, T140L, S141G, S142T, P143M, V154G, Q156C, S168K, S173G, A175Q, G179R, F185Y, R190K, L201C, Q206C, T240A, P252Q, G255A, S262A, H263Q, Y277H, T282A, E284Y, S288A, A335M, L342F, E354K, R362D, N366Y, K385A, T400C, and G417Q;
d) P100C, A131C, Q156C, L201C, Q206C, and T400C;
e) P26G, S37M, K65A, L88W, V89M, A95Q, P100C, S102P, D110N, E120A, L126A, A131C, T140M, S142T, P143M, V154G, Q156C, S173G, A175Q, G179R, R190K, L201C, Q206C, T240A, P252Q, S262A, H263Q, Y277H, T282A, E284Y, S288A, E354K, R362D, N366Y, T400C, and G417Q;
f) P26G, S37M, T48P, D57S, A58P, K65A, L66A, V89R, A90Q, A95Q, P100C, S102A, D110N, G119A, E120A, L126A, A131C, T140L, S141G, S142T, P143M, V154G, Q156C, A160K, N161Q, S168K, S173G, A175Q, T178H, G179E, H180R, T183A, F185Y, R190K, L201C, Q206C, T240A, P252Q, G255A, S262A, H263Q, N266A, T267R, Y277H, Q280F, T282A, E284Y, R287A, S288A, T291G, M298Q, K308N, A335M, E337G, L342F, E354A, R362D, S364A, N366Y, S367K, F376Y, K385A, N391A, T392Q, T400C, and G417Q;
g) P26G, S37M, L88W, V89M, A95Q, P100C, S102P, D110N, E120A, L126A, A131C, T140M, P143M, V154G, Q156C, S173G, A175Q, G179R, R190K, L201C, Q206C, T240A, P252Q, S262A, H263Q, Y277H, T282A, E284Y, R362D, N366Y, T400C, and G417Q;
h) P26G, S37M, T48P, K65A, L88W, V89M, A90Q, A95Q, P100C, S102A, D110N, E120A, L126A, A131C, T140L, S141G, S142K, P143M, V154G, Q156C, S168K, S173G, A175Q, G179R, F185Y, R190K, L201C, Q206C, T240A, P252Q, G255A, S262A, H263Q, Y277H, T282A, E284Y, S288A, A335M, L342F, E354K, R362D, N366Y, K385A, T400C, and G417Q;
i) P26G, E27D, S37M, A47P, T48P, L50T, D53R, V54M, D57R, A58P, T61Q, K65A, L66A, Q84F, L88M, V89R, A90Q, D91Q, A95Q, P100C, S102D, I108W, D110N, E113Q, G119A, E120A, F122Y, L126A, D129G, A131C, I132V, T133R, T140M, S141G, S142T, P143M, V154G, Q156C, A160Q, N161Q, V162A, D164A, I166V, S168K, S173G, A175Q, D176A, F177E, T178H, G179E, H180R, T183A, F185Y, E187Q, R190K, L201C, Q206C, S234A, L235R, T240A, P252Q, E253D, G255A, T260H, S262E, H263Q, N266A, T267R, S270A, Y277H, Q280F, T282A, E284Y, R287A, S288A, D295Q, L296F, M298Q, A299T, K308N, Q309N, T314A, L315H, L320G, I322M, A335M, E337G, L342F, E354A, R362D, L363K, S364A, D365A, N366Y, S367K, I370V, S373A, F376Y, M382L, D384Q, K385A, N391A, T392Q, T400C, A402Q, A416D, and G417R;
j) P26G, E27D, S37M, A47P, T48P, D57R, A58P, T61Q, K65A, L66A, V89R, A90Q, A95Q, P100C, S102D, D110N, E113Q, G119A, E120A, L126A, A131C, T133R, T136Y, T140M, S141G, S142T, P143M, V154G, Q156C, A160K, N161Q, T163V, S168K, S173G, A175Q, D176A, F177E, T178H, G179E, H180R, T183A, F185Y, R190K, L201C, Q206C, L235R, T240A, P252Q, E253D, G255A, S262E, H263Q, N266A, T267R, L269M, Y277H, Q280F, T282A, E284Y, R287A, S288A, P292Y, M298Q, K308N, I322M, A335M, E337G, L342F, E354A, R362D, L363K, S364A, N366Y, S367K, I370V, S373A, F376Y, K385A, N391A, T392Q, T400C, and G417Q;
k) P26G, E27D, S37M, A47P, T48P, L50T, D53R, V54M, D57R, A58P, T61Q, K65A, L66A, Q84F, L88M, V89R, A90Q, D91Q, A95Q, P100C, S102D, D110N, E113Q, G119A, E120A, F122Y, L126A, D129G, A131C, I132V, T133R, T140M, S141G, S142T, P143M, V154G, Q156C, A160Q, N161Q, V162A, D164A, I166V, S168K, S173G, A175Q, D176A, F177E, T178H, G179E, H180R, T183A, F185Y, E187Q, R190K, L201C, Q206C, S234A, L235R, T240A, P252Q, E253D, G255A, T260H, S262E, H263Q, N266A, T267R, S270A, Y277H, Q280F, T282A, E284Y, R287A, S288A, T291G, D295R, L296F, M298Q, A299T, K308N, Q309N, T314A, L320G, I322M, A335M, E337G, L342F, E354A, R362D, L363K, S364A, D365A, N366Y, S367K, I370V, S373A, F376Y, M382L, D384Q, K385A, N391A, T392Q, T400C, A402Q, A416D, and G417R;
l) P26G, S37M, L88W, V89M, A95Q, P100C, S102P, D110N, E120A, L126A, A131C, T140M, P143M, V154G, Q156C, S173G, A175Q, G179R, R190K, L201C, Q206C, T240A, P252Q, S262A, H263Q, Y277H, T282A, E284Y, L315H, R362D, N366Y, T400C, and G417Q;
m) P26G, S37M, T48P, D57S, A58P, K65A, L66A, V89R, A90Q, A95Q, P100C, S102A, D110N, G119A, E120A, L126A, A131C, T140M, S141G, S142K, P143M, V154G, Q156C, A160K, N161Q, S168K, S173G, A175Q, T178H, G179E, H180R, T183A, F185Y, R190K, L201C, Q206C, T240A, P252Q, G255A, S262A, H263Q, N266A, T267R, Y277H, Q280F, T282A, E284Y, R287A, S288A, T291G, M298Q, K308N, A335M, E337G, L342F, E354A, R362D, S364A, N366Y, S367K, F376Y, K385A, N391A, T392Q, T400C, and G417Q; or
n) P26G, S37M, T48P, K65A, L66A, V89R, A90Q, A95Q, P100C, S102A, D110N, E120A, L126A, A131C, T140M, S141G, S142T, P143M, V154G, Q156C, N161Q, S168K, S173G, A175Q, G179E, T183E, F185Y, R190K, L201C, Q206C, T240A, P252Q, G255A, S262E, H263Q, T267R, Y277H, T282A, E284Y, R287A, S288A, A335M, E354K, R362D, S364A, N366Y, S367K, F376Y, K385A, T392Q, T400C, G417Q.

The present invention provides a phytase variant of a wild-type phytase from Buttiauxella sp. of SEQ ID NO: 26 comprising one or more mutations selected from S39P, S39A, G40D, T43V, T43L, S50M, M60S, M64L, R65D, R65Q, D66K, V67W, L79P, I82L, E87A, Q88H, I90M, R91A, V92Q, V92A, F96Y, R98H, E99R, K100W, I106L, I106L, Q109A, N111G, P113C, S117T, D123N, V124I, L129E, G132A, E133Q, E133A, L139A, G144C, N152D, A156M, K164I, I167D, S169C, N173E, N173E, N173Q, I175A, E182R, Q184H, Q184G, N185G, P186S, L195A, N202A, N202D, N202E, T203E, T203Q, Q214C, D219C, I234L, I247L, G248A, L249I, E260Q, Y261Q, P266A, P266Q, K267D, K267N, N272R, S275D, E278Q, A281K, K284A, T288A, A294N, I299V, P306N, L308M, A310Y, I311L, N313G, N318Q, N318H, A319P, I327D, S328A, P329L, P329H, P329Q, N331A, F335V, F335M, F335L, I336L, N345A, S347A, N351G, S353H, T355N, L374W, A375R, S378A, K380T, Y382W, S384R, Q411R, K413N, A422P, E423D, P429S, P429E, T432S, M434L, Q437H, Q437E, S438A, V439L, and G442A.

The present invention provides a phytase variant of a wild-type phytase from Buttiauxella sp. of SEQ ID NO: 26 comprising mutations:
a) T43V, S50M, R65D, V67W, L79P, E87A, I90M, R91A, F96Y, R98H, K100W, I106L, N111G, P113C, D123N, G132A, E133Q, L139A, G144C, A156M, K164I, I167D, S169C, N173E, E182R, Q184H, P186S, L195A, T203E, Q214C, D219C, L249I, Y261Q, P266A, K267D, N272R, E278Q, T288A, A310Y, N313G, P329L, F335V, N345A, S347A, N351G, A375R, K380T, S384R, Q411R, K413N, A422P, E423D, P429S, T432S, M434L, Q437H, and G442A;
b) T43L, S50M, R65D, V67W, L79P, E87A, I90M, R91A, F96Y, R98H, K100W, I106L, Q109A, N111G, P113C, D123N, G132A, E133A, L139A, G144C, N152D, A156M, K164I, I167D, S169C, N173E, E182R, Q184H, P186S, L195A, T203E, Q214C, D219C, I234L, I247L, G248A, Y261Q, P266Q, K267D, N272R, E278Q, A281K, T288A, A310Y, N313G, P329H, F335M, I336L, N345A, S347A, N351G, A375R, S378A, K380T, S384R, Q411R, K413N, A422P, E423D, P429E, T432S, M434L, Q437H, and G442A;
c) S39P, G40D, T43V, S50M, M60S, M64L, R65Q, D66K, V67W, L79P, I82L, E87A, I90M, R91A, V92Q, F96Y, E99R, K100W, I106L, Q109A, N111G, P113C, S117T, D123N, L129E, G132A, E133Q, L139A, G144C, N152D, A156M, K164I, I167D, S169C, N173E, I175A, E182R, Q184G, N185G, P186S, L195A, N202A, T203Q, Q214C, D219C, I234L, I247L, G248A, E260Q, Y261Q, P266Q, K267D, N272R, S275D, E278Q, A281K, K284A, T288A, A294N, I299V, P306N, L308M, A310Y, I311L, N313G, N318Q, A319P, I327D, S328A, P329Q, F335L, I336L, N345A, S347A, N351G, S353H, T355N, L374W, A375R, S378A, K380T, S384R, Q411R, K413N, A422P, E423D, P429E, T432S, M434L, Q437E, S438A, V439L, and G442A;
d) T43V, S50M, R65Q, L79P, E87A, I90M, R91A, F96Y, R98H, K100W, I106L, N111G, P113C, G132A, E133Q, L139A, G144C, A156M, K164I, I167D, S169C, N173E, E182R, P186S, L195A, Q214C, D219C, Y261Q, P266Q, K267D, E278Q, A281K, A310Y, N313G, P329H, F335M, K380T, S384R, Q411R, K413N, A422P, E423D, P429S, T432S, M434L, Q437H, and G442A;
e) T43V, S50M, R65D, V67W, L79P, E87A, R91A, F96Y, R98H, K100W, I106L, N111G, P113C, D123N, G132A, E133Q, L139A, G144C, A156M, K164I, I167D, S169C, N173E, E182R, Q184H, P186S, L195A, T203E, Q214C, D219C, L249I, Y261Q, P266Q, K267D, N272R, E278Q, T288A, A310Y, N313G, P329H, F335M, N345A, S347A, N351G, A375R, K380T, S384R, Q411R, K413N, A422P, E423D, P429S, T432S, M434L, Q437H, G442A;
f) S39P, T43V, S50M, M60S, R65D, D66K, V67W, L79P, I82L, E87A, I90M, R91A, F96Y, R98H, K100W, I106L, Q109A, N111G, P113C, D123N, G132A, E133Q, L139A, G144C, N152D, A156M, K164I, I167D, S169C, N173E, I175A, E182R, Q184G, N185G, P186S, L195A, N202D, T203E, Q214C, D219C, I234L, G248A, L249I, E260Q, Y261Q, P266Q, K267D, N272R, S275D, E278Q, T288A, A294N, A310Y, N313G, N318H, S328A, P329Q, N331A, F335M, I336L, N345A, S347A, N351G, T355N, L374W, A375R, S378A, K380T, Y382W, S384R, Q411R, K413N, A422P, E423D, P429E, T432S, M434L, Q437E, and G442A;
g) T43V, S50M, R65D, D66K, V67W, L79P, I82L, E87A, I90M, R91A, F96Y, R98H, K100W, I106L, Q109A, N111G, P113C, D123N, G132A, E133Q, L139A, G144C, N152D, A156M, K164I, I167D, S169C, N173E, E182R, Q184H, N185G, P186S, L195A, N202D, T203E, Q214C, D219C, I234L, G248A, L249I, E260Q, Y261Q, P266A, K267N, N272R, S275D, E278Q, T288A, A294N, A310Y, N313G, N318H, S328A, P329L, N331A, F335M, I336L, N345A, S347A, N351G, T355N, L374W, A375R, S378A, K380T, S384R, Q411R, K413N, A422P, E423D, P429E, T432S, M434L, Q437H, and G442A;
h) P113C, G144C, S169C, Q214C, D219C, Q411R, K413N, A422P, E423D, P429S, T432S, M434L, Q437H, and G442A;
i) T43V, S50M, R65D, D66K, V67W, L79P, I82L, E87A, I90M, R91A, F96Y, R98H, K100W, I106L, Q109A, N111G, P113C, D123N, G132A, E133Q, L139A, G144C, N152D, A156M, K164I, I167D, S169C, N173E, E182R, N185G, P186S, L195A, N202D, T203E, Q214C, D219C, I234L, G248A, L249I, E260Q, Y261Q, P266Q, K267N, N272R, S275D, E278Q, T288A, A294N, A310Y, I311L, N313G, S328A, P329L, F335M, I336L, N345A, S347A, N351G, S353H, T355N, L374W, A375R, S378A, K380T, S384R, Q411R, K413N, A422P, E423D, P429E, T432S, M434L, Q437H, and G442A;
j) T43V, S50M, R65D, L79P, E87A, I90M, R91A, F96Y, R98H, K100W, I106L, N111G, P113C, G132A, E133Q, L139A, G144C, A156M, K164I, I167D, S169C, N173E, E182R, P186S, L195A, Q214C, D219C, I247L, Y261Q, P266A, K267N, E278Q, A281K, A310Y, N313G, P329H, F335M, K380T, S384R, Q411R, K413N, A422P, E423D, P429S, T432S, M434L, Q437H, and G442A;
k) S39P, T43L, S50M, M60S, R65Q, D66K, V67W, L79P, I82L, E87A, I90M, R91A, V92A, F96Y, R98H, K100W, I106L, Q109A, N111G, P113C, D123N, L129E, G132A, E133A, L139A, G144C, N152D, A156M, K164I, I167D, S169C, N173E, I175A, E182R, Q184G, N185G, P186S, L195A, N202E, T203E, Q214C, D219C, I234L, I247L, G248A, E260Q, Y261Q, P266Q, K267D, N272R, S275D, E278Q, T288A, A294N, A310Y, I311L, N313G, A319P, I327D, S328A, P329Q, F335L, I336L, N345A, S347A, N351G, S353H, T355N, L374W, A375R, S378A, K380T, S384R, Q411R, K413N, A422P, E423D, P429E, T432S, M434L, Q437E, and G442A;
l) T43V, S50M, R65D, V67W, L79P, E87A, I90M, R91A, F96Y, R98H, K100W, I106L, Q109A, N111G, P113C, D123N, G132A, E133Q, L139A, G144C, N152D, A156M, K164I, I167D, S169C, N173E, E182R, Q184H, P186S, L195A, T203E, Q214C, D219C, I234L, I247L, G248A, Y261Q, P266Q, K267N, N272R, E278Q, A281K, T288A, A310Y, N313G, P329H, N331A, F335M, I336L, N345A, S347A, N351G, A375R, S378A, K380T, S384R, Q411R, K413N, A422P, E423D, P429E, T432S, M434L, Q437H, and G442A;
m) S39P, T43V, S50M, M60S, R65Q, D66K, V67W, L79P, I82L, E87A, I90M, R91A, V92A, F96Y, R98H, K100W, I106L, Q109A, N111G, P113C, D123N, L129E, G132A, E133Q, L139A, G144C, N152D, A156M, K164I, I167D, S169C, N173E, I175A, E182R, Q184G, N185G, P186S, L195A, N202A, T203Q, Q214C, D219C, I234L, I247L, G248A, E260Q, Y261Q, P266Q, K267N, N272R, S275D, E278Q, T288A, A294N, A310Y, I311L, N313G, A319P, I327D, S328A, P329Q, F335L, I336L, N345A, S347A, N351G, T355N, L374W, A375R, S378A, K380T, S384R, Q411R, K413N, A422P, E423D, P429E, T432S, M434L, Q437E, and G442A;
n) S39P, T43V, S50M, M60S, R65Q, D66K, V67W, L79P, I82L, E87A, I90M, R91A, F96Y, R98H, K100W, I106L, Q109A, N111G, P113C, D123N, L129E, G132A, E133Q, L139A, G144C, N152D, A156M, K164I, I167D, S169C, N173Q, I175A, E182R, Q184G, N185G, P186S, L195A, N202D, T203E, Q214C, D219C, I234L, G248A, E260Q, Y261Q, P266Q, K267N, N272R, S275D, E278Q, T288A, A294N, A310Y, I311L, N313G, S328A, P329Q, F335M, I336L, N345A, S347A, N351G, T355N, L374W, A375R, S378A, K380T, Y382W, S384R, Q411R, K413N, A422P, E423D, P429E, T432S, M434L, Q437E, and G442A;
o) S39A, G40D, T43V, S50M, M60S, M64L, R65Q, D66K, V67W, L79P, I82L, E87A, Q88H, I90M, R91A, V92Q, F96Y, R98H, E99R, K100W, I106L, Q109A, N111G, P113C, S117T, D123N, V124I, L129E, G132A, E133Q, L139A, G144C, N152D, A156M, K164I, I167D, S169C, N173E, I175A, E182R, Q184G, N185G, P186S, L195A, N202A, T203Q, Q214C, D219C, I234L, I247L, G248A, E260Q, Y261Q, P266Q, K267D, N272R, S275D, E278Q, A281K, K284A, T288A, A294N, I299V, P306N, L308M, A310Y, I311L, N313G, N318Q, I327D, S328A, P329Q, F335L, I336L, N345A, S347A, N351G, T355N, L374W, A375R, S378A, K380T, S384R, Q411R, K413N, A422P, E423D, P429E, T432S, M434L, Q437E, S438A, V439L, and G442A.

The present invention provides a polygalacturonase variant of a wild-type polygalacturonase from Aspergillus niger of SEQ ID NO: 27 comprising one or more mutations selected T36C, A40H, E42D, A43E, E45K, S46A, I47Q, S48Q, S51C, S69K, A71L, D73S, T78I, M106Q, D115G, D115Q, S122G, D139K, V140L, F145I, K146T, K146S, I148L, I158V, V165L, N168D, D179S, G187A, G195Y, I210C, I212V, S217H, S217N, S226C, K245Q, S250T, S252N, S252C, S255T, D256N, A258T, A258D, I266N, K268G, E269G, D272S, E275N, I276F, Q282I, I286C, S301G, S307T, I310V, D314N, V315F, G319N, L324V, E325A, E325S, D326S, Q330N, D343N, S352T, A356T, D358T, and D359K.

The present invention provides a polygalacturonase variant of a wild-type polygalacturonase from Aspergillus niger of SEQ ID NO: 27 comprising mutations:
a) T36C, A43E, S48Q, S51C, A71L, D73S, T78I, D115G, S122G, V140L, K146T, I148L, N168D, D179S, G187A, G195Y, I210C, S217H, S226C, K245Q, S250T, S252N, D256N, A258T, I266N, K268G, E269G, D272S, E275N, Q282I, I286C, S301G, V315F, G319N, L324V, E325A, D326S, Q330N, A356T, and D359K;
b) T36C, A43E, S51C, V140L, K146S, I148L, I210C, S217N, S226C, K245Q, A258T, I266N, K268G, E269G, D272S, Q282I, S301G, G319N, L324V, D326S, A356T, and D359K;
c) T36C, A40H, E42D, A43E, E45K, S46A, I47Q, S48Q, S51C, S69K, A71L, D73S, T78I, M106Q, D115G, S122G, D139K, V140L, F145I, K146T, I148L, I158V, V165L, N168D, D179S, G187A, I210C, I212V, S217N, S226C, K245Q, S250T, S252C, D256N, A258D, I266N, K268G, E269G, D272S, E275N, I276F, Q282I, I286C, S301G, I310V, D314N, V315F, G319N, L324V, E325A, D326S, Q330N, D343N, S352T, A356T, D358T, and D359K;
d) T36C, S51C, I210C, S226C, A356T, and D359K;
e) T36C, A43E, S48Q, S51C, A71L, T78I, S122G, V140L, K146T, I148L, G195Y, I210C, S217N, S226C, K245Q, D256N, A258D, I266N, K268G, E269G, D272S, E275N, Q282I, S301G, G319N, L324V, D326S, A356T, and D359K;
f) T36C, A43E, S48Q, S51C, A71L, D73S, T78I, D115G, S122G, V140L, K146T, I148L, N168D, D179S, G187A, G195Y, I210C, S217N, S226C, K245Q, S250T, S252N, D256N, A258D, I266N, K268G, E269G, D272S, E275N, Q282I, I286C, S301G, V315F, G319N, L324V, E325A, D326S, Q330N, A356T, and D359K;
g) T36C, A43E, S51C, V140L, K146T, I148L, I210C, S217N, S226C, K245Q, A258T, I266N, K268G, E269G, D272S, Q282I, S301G, G319N, L324V, D326S, A356T, and D359K;
h) T36C, E42D, A43E, S46A, I47Q, S48Q, S51C, A71L, D73S, T78I, D115G, S122G, V140L, K146T, I148L, N168D, D179S, G187A, G195Y, I210C, S217N, S226C, K245Q, S250T, S252C, D256N, A258T, I266N, K268G, E269G, D272S, E275N, Q282I, I286C, S301G, I310V, V315F, G319N, L324V, E325A, D326S, Q330N, D343N, S352T, A356T, and D359K;
i) T36C, A43E, S48Q, S51C, A71L, D73S, T78I, D115Q, S122G, V140L, K146T, I148L, N168D, G187A, G195Y, I210C, S217N, S226C, K245Q, D256N, A258D, I266N, K268G, E269G, D272S, E275N, Q282I, I286C, S301G, G319N, L324V, E325S, D326S, Q330N, A356T, and D359K;
j) T36C, A40H, E42D, A43E, E45K, S46A, I47Q, S48Q, S51C, S69K, A71L, D73S, T78I, M106Q, D115Q, S122G, D139K, V140L, F145I, K146T, I148L, I158V, V165L, N168D, D179S, G187A, G195Y, I210C, S217N, S226C, K245Q, S250T, S252C, S255T, D256N, A258T, I266N, K268G, E269G, D272S, E275N, I276F, Q282I, I286C, S301G, S307T, I310V, D314N, V315F, G319N, L324V, E325A, D326S, Q330N, D343N, S352T, A356T, D358T, and D359K; or
k) T36C, A43E, S48Q, S51C, A71L, D73S, T78I, S122G, V140L, K146T, I148L, N168D, G187A, G195Y, I210C, S217N, S226C, K245Q, D256N, A258D, I266N, K268G, E269G, D272S, E275N, Q282I, I286C, S301G, G319N, L324V, E325S, D326S, Q330N, A356T, and D359K.

The present invention provides a sulfhydryl oxidase variant of a wild-type sulfhydryl oxidase from Saccharomyces cerevisiae of SEQ ID NO: 28 comprising one or more mutations selected from D74N, D75E, K76T, V77T, K79E, V81L, Y88F, L92Y, L92M, E99N, R105Q, E106K, H109K, H109R, T110Q, T110N, G113H, G113E, G113R, G113N, Y115F, E117K, E117R, S125A, Y126K, Y126E, V129Q, E133K, V137P, T143E, M147Q, Y159R, K161G, D163P, and A168S.

The present invention provides a sulfhydryl oxidase variant of a wild-type sulfhydryl oxidase from Saccharomyces cerevisiae of SEQ ID NO: 28 comprising mutations:
a) D75E, K76T, K79E, V81L, L92Y, E99N, H109K, G113H, Y115F, E117K, S125A, Y126K, V129Q, E133K, T143E, Y159R, and D163P;
b) D75E, K76T, K79E, V81L, L92M, H109K, G113H, S125A, Y126E, V129Q, and D163P;
c) D75E, K76T, V77T, K79E, V81L, L92M, E99N, H109K, T110Q, G113E, Y115F, E117K, S125A, Y126K, V129Q, E133K, T143E, M147Q, Y159R, K161G, and D163P;
d) D75E, K76T, K79E, V81L, L92M, H109K, G113H, S125A, Y126K, V129Q, and D163P;
e) D75E, H109K, G113R, S125A, Y126K, V129Q, and D163P;
f) H109K, G113R, S125A, Y126E, V129Q, and D163P;
g) H109K, G113R, S125A, Y126K, V129Q, and D163P;
h) D74N, D75E, K76T, V77T, K79E, V81L, Y88F, L92M, E99N, H109K, T110Q, G113H, Y115F, E117K, S125A, Y126K, V129Q, E133K, V137P, T143E, M147Q, Y159R, K161G, and D163P;
i) D74N, D75E, K76T, V77T, K79E, V81L, Y88F, L92Y, E99N, R105Q, H109R, T110N, G113H, Y115F, E117R, S125A, Y126K, V129Q, E133K, V137P, T143E, M147Q, Y159R, K161G, D163P, and A168S;
j) D75E, K76T, K79E, V81L, L92Y, E99N, H109K, G113N, Y115F, E117K, S125A, Y126E, V129Q, E133K, T143E, Y159R, and D163P;
k) D74N, D75E, K76T, V77T, K79E, V81L, Y88F, L92Y, E99N, H109K, T110Q, G113E, Y115F, E117K, S125A, Y126K, V129Q, E133K, V137P, T143E, M147Q, Y159R, K161G, and D163P;
l) D74N, D75E, K76T, V77T, K79E, V81L, Y88F, L92Y, E99N, R105Q, E106K, H109K, T110Q, G113E, Y115F, E117K, S125A, Y126K, V129Q, E133K, V137P, T143E, M147Q, Y159R, K161G, D163P, and A168S; or
m) D75E, K76T, V77T, K79E, V81L, L92M, E99N, H109K, T110Q, G113N, Y115F, E117K, S125A, Y126K, V129Q, E133K, T143E, M147Q, Y159R, K161G, and D163P.

The present invention provides a lipase variant of a wild-type lipase from Thermomyces lanuginosus of SEQ ID NO: 35 comprising one or more mutations selected from E23D, F29Y, N30D, N33K, Q37H, S39A, A52P, N55K, A62N, L74I, V82L, G113A, N116D, D118L, K120T, E121D, N123S, R130K, G131A, D133R, D133K, T136Y, T136W, S137N, S137D, S137E, S138A, A143R, Q148A, H157Y, K259V, I260C, A265S, Q271G, P272T, L286V, and L286I.

The present invention provides a lipase variant of a wild-type lipase from Thermomyces lanuginosus of SEQ ID NO: 35 comprising mutations:
a) F29Y, N30D, N33K, Q37H, S39A, N55K, A62N, L74I, G113A, N116D, D118L, K120T, E121D, N123S, R130K, G131A, D133R, T136Y, S137N, S138A, Q148A, H157Y, K259V, A265S, P272T, and L286V;
b) F29Y, N30D, N33K, Q37H, S39A, N55K, A62N, L74I, G113A, N116D, D118L, K120T, E121D, N123S, R130K, G131A, D133R, T136W, S137D, S138A, Q148A, H157Y, K259V, A265S, P272T, and L286V;
c) F29Y, N33K, Q37H, L74I, G113A, T136W, S137E, H157Y, and K259V;
d) F29Y, N33K, Q37H, S39A, L74I, G113A, T136Y, S137N, H157Y, and K259V;
e) E23D, F29Y, N30D, N33K, Q37H, S39A, A52P, N55K, A62N, L74I, V82L, G113A, N116D, D118L, K120T, E121D, N123S, R130K, G131A, D133R, T136Y, S137N, S138A, A143R, Q148A, H157Y, K259V, I260C, A265S, Q271G, P272T, and L286I;
f) F29Y, N30D, N33K, Q37H, S39A, N55K, A62N, L74I, G113A, K120T, N123S, R130K, D133R, T136Y, S137N, H157Y, and K259V;
g) F29Y, N33K, Q37H, S39A, N55K, L74I, G113A, N123S, D133R, T136Y, S137N, H157Y, and K259V;
h) F29Y, N30D, N33K, Q37H, S39A, N55K, A62N, L74I, G113A, E121D, N123S, R130K, D133K, T136Y, S137N, H157Y, and K259V; or
i) F29Y, N33K, Q37H, L74I, G113A, T136W, S137N, H157Y, and K259V.

The present invention provides a glucoamylase variant of a wild-type glucoamylase from Talaromyces islandicus of SEQ ID NO: 36 comprising one or more mutations selected from A38E, V43I, S44A, N46Q, Y58K, S61G, I66A, T73K, L90A, R98I, N99A, L102H, L102N, T106Q, V107T, E109Q, D113Q, D113E, T122S, E130D, A132G, D148G, L165M, D167A, E174A, Y177N, S178R, V182S, T183N, E198N, E198A, M214S, S215H, A220M, L 226V, L226V, T231A, T231R, F232L, G234A, V236L, A238K, S241C, W242P, D244C, D244C, P248W, Y253C, D293C, I295A, I295S, I295T, S301C, P302S, P302S, N307T, K309M, V310A, V318I, L321I, I325K, E327S, E327Q, V329K, V329R, V329S, F348Y, S350A, N351T, D383Q, V390T, T397S, F400Y, D402N, T409W, G413E, G413D, Y414F, I415M, S416A, A420Y, T424S, S427N, Y432F, D433N, K434R, S435Y, T438A, E439M, N456M, N457H, M458A, S461N, G462S, I463V, P466A, P466H, A470E, N474S, T475S, S478G, S479T, S481A, T482A, G483T, T486I, T491S, and P492V.

The present invention provides a glucoamylase variant of a wild-type glucoamylase from Talaromyces islandicus of SEQ ID NO: 36 comprising mutations:
a) I66A, R98I, L102H, D113Q, E130D, A132G, L165M, E198N, G234A, A238K, S241C, D244C, P248W, Y253C, D293C, S301C, P302S, K309M, V329K, F348Y, N351T, E439M, N456M, S461N, I463V, P466A, A470E, T475S, S478G, S479T, S481A, T482A, G483T, T486I, T491S, and P492V;
b) V43I, I66A, L90A, L102N, D113Q, E130D, A132G, L165M, S178R, E198N, T231A, G234A, A238K, S241C, W242P, D244C, P248W, Y253C, D293C, I295A, S301C, P302S, K309M, I325K, V329K, F348Y, N351T, T397S, G413E, A420Y, D433N, T438A, E439M, N456M, S461N, I463V, P466A, A470E, T475S, S479T, S481A, G483T, T486I, T491S, and P492V;
c) V43I, N46Q, Y58K, S61G, I66A, T73K, L90A, R98I, N99A, L102H, T106Q, V107T, E109Q, D113Q, T122S, E130D, A132G, D148G, L165M, D167A, E174A, Y177N, S178R, V182S, T183N, E198N, M214S, S215H, A220M, L 226V, T231R, F232L, G234A, V236L, A238K, S241C, W242P, D244C, P248W, Y253C, D293C, I295A, S301C, P302S, N307T, K309M, V310A, V318I, L321I, I325K, E327S, V329K, F348Y, S350A, N351T, D383Q, V390T, T397S, F400Y, D402N, T409W, G413D, Y414F, I415M, S416A, T424S, S427N, Y432F, D433N, K434R, S435Y, T438A, E439M, M458A, S461N, I463V, P466H, A470E, T475S, S479T, G483T, T486I, T491S, and P492V;
d) I66A, L90A, L102N, D113Q, E130D, A132G, L165M, S178R, E198N, G234A, A238K, S241C, D244C, P248W, Y253C, D293C, I295A, S301C, P302S, K309M, I325K, V329K, F348Y, N351T, T397S, E439M, N456M, S461N, I463V, P466A, A470E, T475S, S478G, S479T, S481A, T482A, G483T, T486I, T491S, and P492V;
e) I66A, L90A, L102N, D113Q, E130D, A132G, L165M, S178R, E198N, G234A, A238K, S241C, D244C, P248W, Y253C, D293C, I295S, S301C, P302S, K309M, I325K, V329K, F348Y, N351T, T397S, E439M, N456M, S461N, I463V, P466A, A470E, T475S, S478G, S479T, S481A, T482A, G483T, T486I, T491S, and P492V;
f) V43I, I66A, L90A, L102N, D113E, E130D, A132G, L165M, S178R, E198N, T231A, G234A, A238K, S241C, W242P, D244C, P248W, Y253C, D293C, I295S, S301C, P302S, K309M, I325K, V329R, F348Y, N351T, T397S, G413E, A420Y, D433N, T438A, E439M, N456M, S461N, I463V, P466A, A470E, T475S, S479T, S481A, G483T, T486I, T491S, and P492V;
g) I66A, R98I, L102H, D113Q, E130D, A132G, L165M, E198A, G234A, A238K, S241C, D244C, P248W, Y253C, D293C, S301C, P302S, K309M, V329K, F348Y, N351T, E439M, N456M, S461N, I463V, P466A, A470E, T475S, S478G, S479T, S481A, T482A, G483T, T486I, T491S, and P492V;
h) V43I, N46Q, Y58K, S61G, I66A, T73K, L90A, R98I, N99A, L102H, T106Q, V107T, E109Q, D113E, T122S, E130D, A132G, D148G, L165M, D167A, E174A, Y177N, S178R, V182S, T183N, E198N, M214S, S215H, A220M, L226V, T231R, F232L, G234A, V236L, A238K, S241C, W242P, D244C, P248W, Y253C, D293C, I295A, S301C, P302S, N307T, K309M, V310A, V318I, L321I, I325K, E327S, V329S, F348Y, S350A, N351T, D383Q, V390T, T397S, F400Y, D402N, T409W, G413D, Y414F, I415M, S416A, T424S, S427N, Y432F, D433N, K434R, S435Y, T438A, E439M, M458A, S461N, I463V, P466H, A470E, T475S, S479T, G483T, T486I, T491S, and P492V;
i) V43I, S44A, Y58K, S61G, I66A, L90A, L102N, D113Q, E130D, A132G, D148G, L165M, S178R, E198N, M214S, L226V, T231A, G234A, A238K, S241C, W242P, D244C, P248W, Y253C, D293C, I295A, S301C, P302S, K309M, I325K, E327Q, V329S, F348Y, N351T, T397S, D402N, G413D, A420Y, D433N, T438A, E439M, N456M, S461N, I463V, P466A, A470E, T475S, S479T, S481A, G483T, T486I, T491S, and P492V;
j) V43I, S44A, Y58K, S61G, I66A, T73K, L90A, L102N, D113Q, E130D, A132G, D148G, L165M, E174A, Y177N, S178R, E198N, M214S, S215H, L226V, T231A, G234A, A238K, S241C, W242P, D244C, P248W, Y253C, D293C, I295T, S301C, P302S, N307T, K309M, V310A, V318I, I325K, E327Q, V329S, F348Y, S350A, N351T, V390T, T397S, F400Y, D402N, T409W, G413E, A420Y, D433N, K434R, S435Y, T438A, E439M, N457H, M458A, S461N, I463V, P466H, A470E, T475S, S479T, G483T, T486I, T491S, and P492V;
k) I66A, L102N, D113Q, E130D, A132G, L165M, E198N, G234A, A238K, S241C, D244C, P248W, Y253C, D293C, I295A, S301C, P302S, K309M, V329K, F348Y, N351T, E439M, N456M, S461N, I463V, P466A, A470E, T475S, S478G, S479T, S481A, T482A, G483T, T486I, T491S, and P492V;
l) A38E, V43I, S44A, Y58K, S61G, I66A, T73K, L90A, L102N, D113Q, E130D, A132G, D148G, L165M, E174A, Y177N, S178R, E198A, M214S, S215H, L226V, T231A, G234A, A238K, S241C, W242P, D244C, P248W, Y253C, D293C, I295A, S301C, P302S, N307T, K309M, V310A, V318I, I325K, E327Q, V329S, F348Y, S350A, N351T, V390T, T397S, F400Y, D402N, T409W, G413D, D433N, K434R, S435Y, T438A, E439M, N457H, M458A, S461N, I463V, P466H, A470E, T475S, S479T, G483T, T486I, T491S, and P492V;
m) V43I, S44A, Y58K, S61G, I66A, L90A, L102N, D113E, E130D, A132G, D148G, L165M, S178R, E198N, M214S, L226V, T231A, G234A, A238K, S241C, W242P, D244C, P248W, Y253C, D293C, I295A, S301C, P302S, K309M, I325K, E327Q, V329K, F348Y, N351T, T397S, D402N, G413D, A420Y, D433N, T438A, E439M, N456M, S461N, I463V, P466A, A470E, T475S, S479T, S481A, G483T, T486I, T491S, and P492V; or
n) S241C, D244C, Y253C, D293C, S301C, S461N, G462S, I463V, P466A, A470E, N474S, T475S, S478G, S479T, S481A, T482A, G483T, T486I, T491S, and P492V.

The present invention provides a Cas9 variant of a wild-type Cas9 from Streptococcus pyogenes of SEQ ID NO: 37 comprising one or more mutations selected from K8R, A14D, D27E, E28D, E28N, S33R, S33A, R44K, H45K, S46Y, C84K, S91A, S91A, S91A, N92D, N92E, D99P, S100N, K116R, H117Y, H117G, E118S, D128E, A131E, K135Q, R143I, K145Y, V147A, V 147A, D151K, D151Q, D151E, L162C, L162V, L162I, M165L, V185I, D186Q, I190K, I190N, Q194D, Q194K, Q198N, N206S, A207E, A207T, S208N, S208N, L216F, S217T, S217T, R219K, R224D, N228K, N228E, Q232N, E236Q, N244Q, S249A, L252N, T253Q, S258T, S258K, N259T, A263E, D276E, D277E, D280E, Q285K, Q289E, L295Q, L295N, S301Y, D308G, N313D, E315T, I316E, I316Q, I316N, S324A, E331D, Q334K, T337K, T337E, A341K, A341Q, A341E, L342F, Q346Y, A371T, A371T, S372T, E375D, F379Y, P382K, E392Y, V395K, V395K, V395E, N398D, R399K, I418V, G421Q, R428E, R429N, E431G, D432K, F433Y, D439K, D439E, I447L, T470Q, S473T, E474D, E483D, G489E, A490K, Q493K, Q493N, S494A, F502K, K510D, E520Q, Y521K, V534I, A542E, K550Q, A551Q, A551E, D554E, L555Y, L555H, T559N, T559K, N560Y, R561K, K574R, C578G, F579Y, S585K, D609E, N613D, E615K, E617H, V624I, T626W, E637R, E638Q, E638Q, R639Q, K641S, T642K, A644S, H645D, D649E, K650E, V651Q, V651Q, M652L, Q654R, K656E, K656A, R668K, S679N, K688I, S689D, A693N, A693I, T707S, D711I, D711E, Q713E, G740A, V747I, L751I, V754A, H758Q, N762W, N762S, M785Y, Q798K, Q798D, Q798N, E806D, T808E, T808Q, E813D, K814R, V828T, D829G, Q830E, V842I, A844H, V846I, K859R, R863S, S864R, D865K, E877K, E877P, Q889M, N892D, N892E, F901Y, S913T, L915E, S941N, S941E, D951G, L953R, K965N, V967S, Q975N, Q975D, H989F, T999N, T999N, K1080Q, K1080S, K1080Q, F1084L, V1087I, S1092N, N1119D, T1142N, S1146C, S1163L, S1163P, L1168V, G1169P, S1177Q, S1177K, E1193N, V1194I, K1195R, K1195Q, D1197E, D1197N, S1234Y, S1234Q, V1237I, N1238K, L1242N, K1259H, K1259N, Q1260K, L1261K, F1262Y, Q1265E, E1275N, S1281V, R1283K, V1284H, L1286G, D1288K, D1292E, D1292K, L1295E, S1296E, S1296Q, S1296K, H1315N, H1315N, D1348N, D1348N, A1349G, and A1349G.

The present invention provides a Cas9 variant of a wild-type Cas9 from Streptococcus pyogenes of SEQ ID NO: 37 comprising mutations:
a) A14D, E28D, S33R, H45K, C84K, D99P, H117Y, E118S, D128E, R143I, K145Y, V147A, D151K, V185I, D186Q, I190K, A207E, S208N, S217T, R219K, R224D, N228K, Q232N, E236Q, T253Q, D276E, D280E, Q285K, S301Y, N313D, E315T, I316E, T337K, A341K, L342F, A371T, F379Y, V395K, N398D, G421Q, R428E, R429N, E431G, F433Y, D439K, I447L, E474D, E483D, G489E, A490K, Q493K, E520Q, Y521K, A542E, K550Q, A551Q, T559N, N560Y, S585K, E617H, V624I, T626W, E637R, E638Q, K641S, T642K, K650E, V 651Q, M652L, Q654R, S679N, K688I, S689D, T707S, D711I, G740A, L751I, V754A, N762W, Q798K, T808E, E813D, V828T, D829G, Q830E, V842I, A844H, V846I, R863S, E877K, Q889M, L915E, S941N, V967S, Q975N, H989F, T999N, K1080Q, S1146C, S1163L, L1168V, G1169P, S1177Q, V1194I, K1195R, D1197E, K1259H, S1281V, S1296E, D1348N, and A1349G;
b) A14D, E28D, S33R, H45K, C84K, N92D, D99P, H117Y, E118S, D128E, R143I, K145Y, V147A, D151Q, L162C, M165L, V185I, D186Q, I190K, Q194D, A207E, S208N, S217T, R219K, R224D, N228K, Q232N, E236Q, T253Q, A263E, D276E, D280E, Q285K, S301Y, N313D, E315T, I316Q, Q334K, T337E, A341K, L342F, A371T, F379Y, V395K, N398D, I418V, G421Q, R428E, R429N, E431G, F433Y, D439E, I447L, S473T, E474D, E483D, G489E, A490K, Q493N, S494A, F502K, E520Q, Y521K, V534I, A542E, A551Q, L555Y, T559K, N560Y, R561K, S585K, E617H, V624I, T626W, E637R, E638Q, K641S, T642K, K650E, V651Q, M652L, Q654R, K656E, K688I, S689D, A693N, T707S, D711I, G740A, L751I, V754A, N762W, Q798K, T808E, E813D, K814R, V828T, D829G, V842I, A844H, V846I, R863S, E877K, Q889M, F901Y, L915E, S941N, V967S, Q975D, H989F, T999N, K1080S, F1084L, V1087I, S1146C, S1163L, L1168V, G1169P, S1177Q, E1193N, V1194I, K1195R, D1197E, S1234Y, K1259H, Q1260K, L1261K, S1281V, V1284H, S1296E, H1315N, D1348N, and A1349G;
c) A14D, E28D, S33A, H45K, S46Y, C84K, D99P, H117Y, E118S, D128E, R143I, K145Y, V147A, D151Q, L162C, M165L, V185I, D186Q, I190N, A207T, S208N, S217T, R219K, R224D, N228K, Q232N, E236Q, T253Q, A263E, D276E, D280E, Q285K, L295Q, S301Y, N313D, E315T, I316E, T337K, A341K, L342F, A371T, F379Y, P382K, V395K, N398D, G421Q, R428E, R429N, E431G, F433Y, D439K, I447L, S473T, E474D, E483D, G489E, A490K, Q493N, S494A, E520Q, Y521K, V534I, A542E, K550Q, A551Q, L555Y, T559K, N560Y, S585K, E617H, V624I, T626W, E637R, E638Q, K641S, T642K, K650E, V651Q, M652L, Q654R, K656E, S679N, K688I, S689D, T707S, D711I, G740A, L751I, V754A, N762W, T808E, E813D, V828T, D829G, Q830E, V842I, A844H, V846I, R863S, E877P, Q889M, F901Y, L915E, S941N, V967S, Q975D, H989F, T999N, K1080Q, S1146C, S1163L, L1168V, G1169P, S1177Q, V1194I, K1195R, D1197E, K1259H, Q1260K, S1281V, S1296E, H1315N, D1348N, and A1349G;
d) A14D, E28D, S33R, R44K, H45K, C84K, S91A, N92E, D99P, H117Y, E118S, D128E, R143I, K145Y, V147A, D151E, L162C, M165L, V185I, D186Q, I190K, Q194D, N206S, A207E, S208N, S217T, R219K, R224D, N228K, Q232N, E236Q, S249A, T253Q, S258T, A263E, D276E, D277E, D280E, Q289E, S301Y, N313D, E315T, I316N, S324A, Q334K, T337K, A341Q, L342F, A371T, F379Y, P382K, V395K, N398D, I418V, G421Q, R428E, R429N, E431G, D432K, F433Y, D439E, I447L, T470Q, S473T, E474D, E483D, G489E, A490K, Q493K, S494A, F502K, K510D, E520Q, Y521K, V534I, A542E, K550Q, A551E, D554E, L555H, T559K, N560Y, R561K, C578G, S585K, D609E, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711E, G740A, V747I, L751I, V754A, N762W, M785Y, Q798D, T808E, E813D, K814R, V828T, D829G, Q830E, V842I, A844H, V846I, K859R, R863S, D865K, E877K, Q889M, N892D, F901Y, L915E, S941E, D951G, L953R, V967S, Q975N, H989F, T999N, K1080Q, S1092N, T1142N, S1146C, S1163L, L1168V, G1169P, S1177K, E1193N, V1194I, K1195R, D1197E, S1234Q, K1259N, Q1260K, L1261K, S1281V, V1284H, D1292E, S1296Q, H1315N, D1348N, and A1349G;
e) A14D, E28D, S33R, H45K, C84K, N92D, D99P, H117Y, E118S, D128E, R143I, K145Y, V 147A, D151Q, L162C, M165L, V185I, D186Q, I190K, Q194D, A207E, S208N, S217T, R219K, R224D, N228K, Q232N, E236Q, T253Q, A263E, D276E, D280E, Q285K, L295Q, S301Y, N313D, E315T, I316Q, Q334K, T337K, A341K, L342F, A371T, F379Y, P382K, V395K, N398D, I418V, G421Q, R428E, R429N, E431G, F433Y, D439E, I447L, S473T, E474D, E483D, G489E, A490K, Q493K, S494A, F502K, E520Q, Y521K, V534I, A542E, K550Q, A551Q, L555Y, T559K, N560Y, R561K, S585K, E617H, V624I, T626W, E637R, E638Q, K641S, T642K, K650E, V651Q, M652L, Q654R, K656E, S679N, K688I, S689D, A693N, T707S, D711I, G740A, L751I, V754A, N762W, Q798K, T808E, E813D, K814R, V828T, D829G, Q830E, V842I, A844H, V846I, R863S, E877K, Q889M, F901Y, L915E, S941N, V967S, Q975D, H989F, T999N, K1080S, F1084L, V1087I, S1146C, S1163L, L1168V, G1169P, S1177Q, E1193N, V1194I, K1195R, D1197E, S1234Y, K1259H, Q1260K, L1261K, S1281V, V1284H, S1296E, H1315N, D1348N, A1349G;
f) A14D, E28D, S33R, H45K, C84K, D99P, H117Y, E118S, D128E, R143I, K145Y, V147A, D151Q, L162V, M165L, V185I, D186Q, I190N, A207E, S208N, S217T, R219K, R224D, N228K, Q232N, E236Q, N244Q, T253Q, N259T, A263E, D276E, D280E, Q285K, S301Y, N313D, E315T, I316E, T337K, L342F, A371T, F379Y, V395E, N398D, R399K, G421Q, R428E, R429N, E431G, F433Y, D439K, I447L, S473T, E474D, E483D, G489E, A490K, Q493K, S494A, E520Q, Y521K, V534I, A542E, K550Q, A551E, L555Y, T559K, N560Y, S585K, E617H, V624I, T626W, E637R, E638Q, K641S, T642K, K650E, V651Q, M652L, Q654R, K656E, S679N, K688I, S689D, T707S, D711I, G740A, L751I, V754A, N762W, Q798K, T808E, E813D, V828T, D829G, Q830E, V842I, A844H, V846I, R863S, E877K, Q889M, F901Y, L915E, S941N, V967S, Q975D, H989F, T999N, K1080Q, S1146C, S1163L, L1168V, G1169P, S1177Q, V1194I, K1195R, D1197E, K1259H, Q1260K, S1281V, S1296E, H1315N, D1348N, A1349G;
g) K8R, A14D, E28D, S33R, H45K, C84K, S91A, N92D, D99P, S100N, K116R, H117G, E118S, D128E, R143I, K145Y, V147A, D151Q, L162I, M165L, V185I, D186Q, I190K, Q194D, A207E, S208N, S217T, R219K, R224D, N228K, Q232N, E236Q, T253Q, A263E, D276E, D280E, Q285K, S301Y, N313D, E315T, I316E, Q334K, T337K, A341Q, L342F, A371T, F379Y, P382K, V395E, N398D, R399K, I418V, G421Q, R428E, R429N, E431G, D432K, F433Y, D439E, I447L, S473T, E474D, E483D, G489E, A490K, Q493K, S494A, F502K, E520Q, Y521K, V534I, A542E, K550Q, A551Q, T559K, N560Y, R561K, S585K, E617H, V624I, T626W, E637R, E638Q, K641S, T642K, K650E, V651Q, M652L, Q654R, K656E, R668K, K688I, S689D, A693I, T707S, D711E, G740A, L751I, V754A, H758Q, N762S, Q798K, T808E, E813D, K814R, V828T, D829G, Q830E, V842I, A844H, V846I, R863S, D865K, E877K, Q889M, F901Y, L915E, S941E, L953R, Q975D, H989F, T999N, S1146C, S1163L, L1168V, G1169P, S1177K, E1193N, V1194I, K1195R, D1197E, S1234Q, N1238K, K1259H, Q1260K, L1261K, S1281V, V1284H, S1296E, H1315N, D1348N, and A1349G;
h) K8R, A14D, E28D, S33R, R44K, H45K, C84K, S91A, N92E, D99P, S100N, K116R, H117G, E118S, D128E, R143I, K145Y, V147A, D151E, L162C, M165L, V185I, D186Q, I190K, Q194D, N206S, A207E, S208N, S217T, R219K, R224D, N228K, Q232N, E236Q, S249A, T253Q, S258T, A263E, D276E, D280E, Q285K, S301Y, N313D, E315T, I316N, S324A, Q334K, T337K, A341Q, L342F, Q346Y, A371T, F379Y, P382K, V395K, N398D, I418V, G421Q, R428E, R429N, E431G, D432K, F433Y, D439E, I447L, T470Q, S473T, E474D, E483D, G489E, A490K, Q493K, S494A, F502K, E520Q, Y521K, V534I, A542E, K550Q, A551Q, L555Y, T559K, N560Y, R561K, K574R, S585K, E617H, V624I, T626W, E637R, E638Q, R639Q, K641S, T642K, H645D, D649E, K650E, V651Q, M652L, Q654R, K656E, R668K, S679N, K688I, S689D, A693N, T707S, D711I, G740A, V747I, L751I, V754A, N762W, M785Y, Q798D, T808E, E813D, K814R, V828T, D829G, V842I, A844H, V846I, K859R, R863S, D865K, E877K, Q889M, N892D, F901Y, L915E, S941E, D951G, Q975D, H989F, T999N, K1080Q, S1092N, T1142N, S1146C, S1163P, L1168V, G1169P, S1177K, E1193N, V1194I, K1195R, D1197E, S1234Q, N1238K, K1259N, Q1260K, L1261K, S1281V, V1284H, S1296E, H1315N, D1348N, and A1349G;
i) K8R, A14D, E28N, S33R, R44K, H45K, C84K, S91A, N92D, D99P, S100N, H117Y, E118S, D128E, R143I, K145Y, V147A, D151E, L162V, M165L, V185I, D186Q, I190N, Q194K, N206S, A207E, S208N, S217T, R219K, R224D, N228K, Q232N, E236Q, S249A, T253Q, S258T, A263E, D276E, D280E, Q285K, S301Y, N313D, E315T, I316N, S324A, Q334K, T337K, A341Q, L342F, Q346Y, A371T, F379Y, P382K, V395K, N398D, I418V, G421Q, R428E, R429N, E431G, D432K, F433Y, D439E, I447L, S473T, E474D, E483D, G489E, A490K, Q493K, S494A, F502K, E520Q, Y521K, V534I, A542E, K550Q, A551Q, D554E, L555Y, T559K, N560Y, R561K, S585K, E617H, V624I, T626W, E637R, E638Q, R639Q, K641S, T642K, H645D, D649E, K650E, V651Q, M652L, Q654R, K656A, R668K, K688I, S689D, A693N, T707S, D711I, G740A, V747I, L751I, V754A, H758Q, N762W, M785Y, Q798D, T808E, E813D, K814R, V828T, D829G, Q830E, V842I, A844H, V846I, K859R, R863S, D865K, E877K, Q889M, N892D, F901Y, L915E, S941E, D951G, L953R, V967S, Q975D, H989F, T999N, K1080Q, V1087I, S1092N, T1142N, S1146C, S1163P, L1168V, G1169P, S1177K, E1193N, V1194I, K1195R, D1197E, S1234Q, K1259H, Q1260K, L1261K, S1281V, V1284H, S1296E, H1315N, D1348N, and A1349G;
j) A14D, D27E, E28N, S33R, R44K, H45K, C84K, S91A, N92E, D99P, S100N, K116R, H117G, E118S, D128E, A131E, K135Q, R143I, K145Y, V147A, D151E, L162V, M165L, V185I, D186Q, I190N, Q198N, N206S, A207T, S208N, L216F, S217T, R219K, R224D, N228K, Q232N, E236Q, S249A, L252N, T253Q, S258K, A263E, D276E, D277E, D280E, Q285K, Q289E, L295N, S301Y, N313D, E315T, I316N, S324A, E331D, Q334K, T337K, A341Q, L342F, Q346Y, A371T, S372T, E375D, F379Y, E392Y, V395K, N398D, I418V, G421Q, R428E, R429N, E431G, D432K, F433Y, D439E, I447L, S473T, E474D, E483D, G489E, A490K, Q493K, S494A, F502K, K510D, E520Q, Y521K, V534I, A542E, K550Q, A551Q, L555H, T559K, N560Y, R561K, C578G, F579Y, S585K, D609E, N613D, E615K, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711I, Q713E, G740A, V747I, L751I, V754A, N762W, M785Y, Q798N, E806D, T808Q, E813D, K814R, V828T, D829G, Q830E, V842I, A844H, V846I, K859R, R863S, S864R, D865K, E877K, Q889M, N892E, F901Y, S913T, L915E, S941E, D951G, L953R, K965N, V967S, Q975N, H989F, T999N, K1080S, F1084L, V1087I, S1092N, N1119D, T1142N, S1146C, S1163L, L1168V, G1169P, S1177K, E1193N, V1194I, K1195R, D1197E, S1234Q, V1237I, L1242N, K1259N, Q1260K, L1261K, F1262Y, Q1265E, E1275N, S1281V, R1283K, V1284H, L1286G, D1288K, D1292E, L1295E, S1296K, H1315N, D1348N, and A1349G;
k) A14D, E28D, S33R, H45K, C84K, S91A, N92E, D99P, S100N, K116R, H117G, E118S, D128E, R143I, K145Y, V147A, D151Q, L162C, M165L, V185I, D186Q, I190K, Q194D, A207E, S208N, S217T, R219K, R224D, N228K, Q232N, E236Q, N244Q, T253Q, N259T, A263E, D276E, D280E, Q285K, S301Y, N313D, E315T, I316Q, Q334K, T337K, A341E, L342F, A371T, F379Y, P382K, V395E, N398D, R399K, I418V, G421Q, R428E, R429N, E431G, D432K, F433Y, D439E, I447L, S473T, E474D, G489E, A490K, Q493K, S494A, F502K, E520Q, Y521K, A542E, K550Q, A551Q, L555Y, T559K, N560Y, R561K, S585K, E617H, V624I, T626W, E637R, E638Q, K641S, T642K, K650E, V651Q, M652L, Q654R, K656E, R668K, S679N, K688I, S689D, A693I, T707S, D711E, G740A, L751I, V754A, H758Q, N762S, Q798K, T808E, E813D, K814R, V828T, D829G, V842I, A844H, V846I, R863S, D865K, E877K, Q889M, F901Y, L915E, S941E, L953R, V967S, Q975N, H989F, T999N, S1146C, S1163L, L1168V, G1169P, S1177K, E1193N, V1194I, K1195R, D1197E, S1234Q, K1259N, Q1260K, L1261K, S1281V, V1284H, S1296E, H1315N, D1348N, and A1349G;
l) K8R, A14D, E28D, S33R, H45K, C84K, D99P, H117Y, E118S, D128E, R143I, K145Y, V147A, D151K, L162I, V185I, D186Q, I190N, A207T, S208N, S217T, R219K, R224D, N228K, Q232N, E236Q, T253Q, D276E, D280E, Q285K, S301Y, N313D, E315T, I316Q, T337K, A341K, L342F, A371T, F379Y, V395K, N398D, G421Q, R428E, R429N, E431G, F433Y, D439K, I447L, S473T, E474D, E483D, G489E, A490K, Q493K, E520Q, Y521K, V534I, A542E, K550Q, A551Q, L555Y, T559N, N560Y, S585K, E617H, V624I, T626W, E637R, E638Q, K641S, T642K, K650E, V651Q, M652L, Q654R, S679N, K688I, S689D, T707S, D711I, G740A, L751I, V754A, N762W, Q798K, T808E, E813D, V828T, D829G, V842I, A844H, V846I, R863S, E877K, Q889M, F901Y, L915E, S941N, V967S, Q975D, H989F, T999N, K1080Q, S1146C, S1163L, L1168V, G1169P, S1177Q, V1194I, K1195R, D1197E, K1259H, S1281V, S1296E, D1348N, and A1349G;
m) A14D, E28N, S33R, R44K, H45K, C84K, S91A, N92E, D99P, H117Y, E118S, D128E, K135Q, R143I, K145Y, V147A, D151E, L162V, M165L, V185I, D186Q, I190K, Q194D, Q198N, N206S, A207E, S208N, L216F, S217T, R219K, R224D, N228K, Q232N, E236Q, S249A, L252N, T253Q, S258K, A263E, D276E, D277E, D280E, Q289E, L295N, S301Y, D308G, N313D, E315T, I316N, S324A, E331D, Q334K, T337K, A341Q, L342F, Q346Y, A371T, S372T, E375D, F379Y, P382K, E392Y, V395K, N398D, I418V, G421Q, R428E, R429N, E431G, D432K, F433Y, D439E, I447L, E474D, E483D, G489E, A490K, Q493K, S494A, F502K, K510D, E520Q, Y521K, V534I, A542E, K550Q, A551Q, L555H, T559K, N560Y, R561K, C578G, F579Y, S585K, D609E, N613D, E615K, E617H, V624I, T626W, E637R, E638Q, R639Q, K641S, T642K, A644S, H645D, D649E, K650E, V651Q, M652L, Q654R, K656A, R668K, S679N, K688I, S689D, A693N, T707S, D711I, Q713E, G740A, V747I, L751I, V754A, H758Q, N762W, M785Y, Q798N, E806D, T808Q, E813D, K814R, V828T, D829G, V842I, A844H, V846I, K859R, R863S, S864R, D865K, E877K, Q889M, N892E, F901Y, S913T, L915E, S941E, D951G, L953R, K965N, V967S, Q975D, H989F, T999N, F1084L, V1087I, S1092N, N1119D, T1142N, S1146C, S1163L, L1168V, G1169P, S1177K, E1193N, V1194I, K1195Q, D1197N, S1234Q, V1237I, N1238K, K1259H, Q1260K, L1261K, F1262Y, Q1265E, E1275N, S1281V, R1283K, V1284H, L1286G, D1292K, S1296Q, H1315N, D1348N, and A1349G; or
n) A14D, E28D, S33R, R44K, H45K, C84K, S91A, N92E, D99P, S100N, H117Y, E118S, D128E, R143I, K145Y, V147A, D151E, L162V, M165L, V185I, D186Q, I190K, Q194D, N206S, A207E, S208N, S217T, R219K, R224D, N228E, Q232N, E236Q, S249A, T253Q, S258T, A263E, D276E, D277E, D280E, Q285K, Q289E, S301Y, D308G, N313D, E315T, I316N, S324A, Q334K, T337K, A341Q, L342F, Q346Y, A371T, F379Y, V395K, N398D, I418V, G421Q, R428E, R429N, E431G, D432K, F433Y, D439E, I447L, T470Q, S473T, E474D, E483D, G489E, A490K, Q493K, S494A, F502K, K510D, E520Q, Y521K, V534I, A542E, K550Q, A551Q, D554E, L555H, T559K, N560Y, R561K, C578G, S585K, D609E, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711I, G740A, V747I, L751I, V754A, H758Q, N762W, M785Y, Q798D, T808E, E813D, K814R, V828T, D829G, Q830E, V842I, A844H, V846I, K859R, R863S, D865K, E877K, Q889M, N892E, F901Y, L915E, S941E, D951G, V967S, Q975N, H989F, T999N, K1080Q, V1087I, S1092N, T1142N, S1146C, S1163L, L1168V, G1169P, S1177K, E1193N, V1194I, K1195R, D1197E, S1234Q, L1242N, K1259N, Q1260K, L1261K, S1281V, V1284H, D1292E, S1296Q, H1315N, D1348N, and A1349G.

The present invention also provides a Cas9 variant of a wild-type Cas9 from Campylobacter jejuni of SEQ ID NO: 38 comprising one or more mutations selected from A2K, A6G, D117E, K155L, A157S, S228E, K288D, A293K, T303K, T309I, I366E, I366D, K367E, S384K, F398L, L403K, V404L, V404I, T405I, T405V, L409E, E410Q, and N477H.

The present invention also provides a Cas9 variant of a wild-type Cas9 from Campylobacter jejuni of SEQ ID NO: 38 comprising mutations:
a) A6G, T309I, F398L, V404L, and T405I;
b) A6G, D117E, K155L, A157S, T303K, T309I, I366E, S384K, F398L, V404I, T405V, and N477H;
c) A6G, T309I, I366E, F398L, V404I, T405V, and N477H;
d) A6G, T309I, F398L, V404I, T405I, and N477H;
e) A6G, D117E, T309I, I366E, S384K, F398L, V404I, T405V, and N477H;
f) A6G, D117E, K155L, T309I, I366E, S384K, F398L, V404I, T405V, and N477H;
g) A2K, A6G, D117E, K155L, A157S, S228E, K288D, A293K, T303K, T309I, I366D, K367E, S384K, F398L, L403K, V404I, T405V, L409E, E410Q, and N477H, or
h) A6G, D117E, K155L, T309I, I366E, S384K, F398L, V404I, T405I, and N477H.

Each of the enzyme variants provided herein retain at least 95% activity, at least 90% activity, at least 85% activity, at least 80% activity, or at least 75% activity, after 4 days at 20° C. and at a pH in the range of 3.5-8, such as a pH in the range of 5-7.

Other features and advantages of the present invention will become apparent from the following detailed description, and examples. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

### Alpha-Amylase Variants

Alpha-amylase is an enzyme that hydrolyzes polysaccharides, such as starch and glycogen, and is found in bacteria, prokaryotes called archaea, fungi, plants, seeds/grain, and animals. In humans, there are two types of alpha-amylase: one secreted by the salivary glands, which is called ptyalin, and has an optimum pH of about 5.6-7, and the second secreted by the pancreas, called pancreatic alpha-amylase, and has an optimum pH of about 7.5-8. Salivary alpha-amylase cleaves starch into dextrins and maltose, a disaccharide. Salivary alpha-amylase is inactivated in the stomach by gastric acid, for example at a pH of 3.3 or lower, however, the presence of starch and glucose, one of the products of alpha-amylase digestion, protects against complete inactivation of this enzyme.

Alpha-amylase has numerous industrial applications. It is used for fermentation in brewing alcohol, such as beer and liquor. In brewing, alpha-amylase has an optimum pH of 5.5 and an optimum temperature of about 70° C. In the production of high-fructose corn syrup, alpha-amylase is used in the first step to convert cornstarch into oligosaccharides. Alpha-amylase is used in baking bread and baked goods to degrade starch in flour and in dough containing yeast, as well as to provide good texture and anti-staling properties of the baked products. Alpha-amylase is also added to detergents, desizing a cotton fabric and animal feed to break down starch.

The alpha-amylase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type alpha-amylases will denature, decompose and/or under which the alpha-amylase enzymatic function is significantly reduced or eliminated.

The present invention provides an alpha-amylase variant of a wild-type alpha-amylase from Bacillus amyloliquefaciens of SEQ ID NO: 1 comprising mutations:
(a) K7N, L18M, V24I, T35S, N40S, V42D, A43N, N49D, T51N, T64K, Q65E, K68Q, R71A, Q91H, D93N, R97E, L102H, S107D, E120D, A127E, V135I, D147H, A149N, R157N, E194Q, T197E, R201G, F202Y, L203F, R205Q, L207V, N208A, P225T, S232G, R233N, T241N, T259A, S278E, S288A, S291E, D296S, S298P, D300S, T304L, I327L, G335A, S338A, G339S, E349Y, R356M, G359N, R366E, A369D, F371W, T377A, Q381K, Q406Y, S409N, T423N, and V424L;
(b) A43T, N49D, T51N, Q65E, R71A, Q91H, D93N, D147H, R233N, S291E, D296S, I327M, E349Y, R356M, R366E, A369D, T377A, and Q406Y;
(c) L18M, V42D, A43T, N49D, T51N, Q65E, R71A, Q91H, D93N, D147H, R157N, R233N, S291E, D296S, I327L, E349Y, R356M, G359N, R366E, A369D, T377A, Q406Y, S409N, and T423D;
(d) L18M, N40S, V42D, A43T, N49D, T51N, T64K, Q65E, R71A, Q91H, D93N, D147H, R157N, S232G, R233N, T241N, S288K, S291E, D296S, D300S, I327L, E349Y, R356M, G359N, R366E, A369D, T377A, Q381K, Q406Y, S409N, and T423D;
(e) L18M, N40S, V42D, A43T, N49D, T51N, T64K, Q65E, R71A, Q91H, D93N, A127E, D147H, A149N, R157N, P225T, S232G, R233N, T241N, T259A, S288K, S291E, D296S, D300S, I327L, G339S, E349Y, R356M, G359N, R366E, A369D, T377A, Q381K, Q406Y, S409N, and T423D;
(f) K7N, L18M, V24I, T35S, N40S, V42D, A43N, N49D, T51N, T64K, K68Q, R71A, Q91H, D93N, R97E, L102H, S107D, E120D, A127E, V135I, D147H, A149N, R157N, E194Q, T197E, R201G, F202Y, R205Q, N208A, P225T, S232G, R233N, T241N, T259A, S278E, S288A, S291A, D296S, S298P, D300S, T304L, I327L, G335A, S336A, S338A, G339S, E349Y, R356M, G359N, R366E, A369D, F371W, T377A, Q381K, Q406Y, S409N, T423N, and V424L;
(g) K7N, L18M, V24I, T35S, N40S, V42D, A43N, N49D, T51N, T64K, K68Q, R71A, Q91H, D93N, R97E, L102H, S107D, E120D, A127E, V135I, D147H, A149N, R157N, E194Q, T197E, R201G, F202Y, R205Q, N208A, P225T, S232G, R233N, T241N, T259A, S278E, S288A, S291A, D296S, S298P, D300S, T304L, I327L, G335A, S336A, S338A, G339S, E349Y, R356M, G359N, R366E, A369D, F371W, T377A, Q381K, Q406Y, S409N, T423N, and V424L;
(h) A43T, N49D, T51N, Q65E, R71A, Q91H, D93N, D147H, R233N, S291E, D296S, I327L, E349Y, R356M, R366E, A369D, T377A, and Q406Y; or
(i) A43T, N49D, T51N, Q65E, R71A, Q91H, D93N, D147H, R233N, S291E, D296S, I327L, A369D, T377A, and Q406Y.

The present invention also provides an alpha-amylase variant of a wild-type alpha-amylase from Bacillus licheniformis of SEQ ID NO: 2 comprising mutations:
(a) K48H, S49N, L63A, K64A, H65Q, D72E, K87H, Y100W, S107K, Q109T, R113N, Y114Q, T117S, V133L, K134Q, V137A, S197Q, K200I, E204N, T205D, A206C, T241N, K281A, S286N, S291G, A294Q, S295N, S340G, V355T, T381H, E392F, I402T, I412F, V427T, S428A, S435R, R448N, and L456I;
(b) K48H, S49N, L63A, H65Q, K87H, Y100W, S107K, Q109S, R113N, Y114Q, T117S, K134Q, V137A, S197Q, K200H, E204N, T205D, K281A, S286N, A294W, S295N, S340G, T381H, E392F, I412F, V427T, S428A, S435R, R448N, and L456I;
(c) L63A, H65Q, K87H, Y100W, S107K, Q109S, R113N, T117S, E129H, K134Q, V137A, S197Q, E204N, T205D, K281A, S286N, A294W, S295N, S340G, T381H, E392F, I412F, V427T, S428A, S435R, R448N, and L456I;
(d) K48H, S49N, L63A, K64Q, H65Q, D72E, K87H, Y100W, S107K, Q109S, R113N, Y114Q, T117S, A127E, E130K, V133L, K134Q, V137A, W175R, S197Q, K200I, F202Y, E204N, T205D, A206C, N208S, F216Y, N229G, T241N, K281E, S286N, S291G, A294Q, S295N, S340G, V355T, Q374K, T381H, P390D, E392F, I402T, I412F, V427T, S428A, S435R, R448N, and L456I;
(e) K48Q, L63A, H65Q, K87H, Y100W, S107K, Q109S, R113N, T117S, E129H, K134Q, V137A, S197Q, E204N, T205D, K281A, S286N, A294W, S295N, S340G, T381H, E392F, I412F, V427T, S428A, S435R, R448N, and L456I;
(f) K48H, S49N, L63A, K64A, H65Q, D72E, K87H, Y100W, S107K, Q109S, R113N, Y114Q, T117S, E129H, V133L, K134Q, V137A, S197Q, K200I, E204N, T205D, A206C, T241N, K281E, S286N, S291G, A294Q, S295N, S340G, V355T, T381H, I402T, Q406R, I412F, V427T, S428A, S435R, R448N, and L456I;
(g) K48H, S49N, L63A, H65Q, K87H, Y100W, S107K, Q109S, R113N, Y114Q, T117S, K134Q, V137A, S197Q, E204N, T205D, K281A, S286N, S295N, S340G, T381H, E392F, I402T, I412F, V427T, S428A, S435R, R448N, and L456I;
(h) K48H, L63A, H65Q, K87H, Y100W, S107K, Q109S, R113N, T117S, E129H, K134Q, V137A, S197Q, E204N, T205D, K281A, S286N, S295N, S340G, T381H, E392F, I412F, V427T, S428A, S435R, R448N, and L456I;
(i) V427T, S428A, S435R, R448N, and L456I;
(j) K48H, S49N, L63A, H65Q, K87H, Y100W, S107K, Q109S, R113N, Y114Q, T117S, K134Q, V137A, S197Q, E204N, T205D, K281A, S286N, A294W, S295N, S340G, T381H, E392F, I402T, I412F, V427T, S428A, S435R, R448N, and L456I;
(k) K48H, S49N, L63A, H65Q, K87H, Y100W, S107K, Q109S, R113N, Y114Q, T117S, K134Q, V137A, S197Q, K200I, E204N, T205D, K281A, S286N, A294W, S295N, S340G, V355T, T381H, E392F, I402T, I412F, V427T, S428A, S435R, R448N, and L456I;
(l) K48H, S49N, L63A, H65Q, K87H, Y100W, S107K, Q109S, R113N, Y114Q, T117S, K134R, V137A, S197Q, K200I, E204N, T205D, K281A, S286N, A294W, S295N, S340G, V355T, T381H, I402T, Q406R, I412F, V427T, S428A, S435R, R448N, and L456I;
(m) K48H, L63A, H65Q, K87H, Y100W, S107K, Q109S, R113N, T117S, K134Q, V137A, S197Q, E204N, T205D, K281A, S286N, A294W, S295N, S340G, T381H, E392F, I412F, V427T, S428A, S435R, R448N, and L456I;
(n) K48H, S49N, L63A, H65Q, K87H, Y100W, S107K, Q109T, R113N, Y114Q, T117S, K134R, V137A, S197Q, K200H, E204N, T205D, K281A, S286N, A294W, S295N, S340G, T381H, Q406R, I412F, V427T, S428A, S435R, R448N, and L456I; or
(o) K48H, S49N, L63A, K64Q, H65Q, D72E, K87H, Y100W, S107K, Q109S, R113N, Y114Q, T117S, A127E, E129H, E130K, V133L, K134Q, V137A, W175R, S197Q, K200A, F202Y, E204N, T205D, A206C, N208S, F216Y, N229G, T241N, K281E, S286N, S291G, A294Q, S295N, S340G, V355T, Q374K, T381H, P390D, I402T, Q406R, I412F, V427T, S428A, S435R, R448N, and L456I.

The present invention further provides an alpha amylase variant of a wild-type alpha amylase from Thermoactinomyces of SEQ ID NO: 17 comprising mutations:
a) L2N, F7Y, E9R, A10P, S13N, S19D, T21N, Q22H, R24V, L27I, A29T, V35K, L40H, A42G, E50Q, A55E, L56M, G58K, S62E, L72E, S76P, T87E, A93R, A103K, A157S, R178K, P180D, E183K, E184D, A197K, H201N, L210F, A211K, P220E, D227E, R253Q, Q257K, S276K, S279P, R280E, V288K, Q289H, A292H, F313Y, N328Y, A333D, R340K, S344E, G361P, M364Q, S379A, T386K, E388K, H390T, R393Q, A396H, T399N, A410N, G412A, A434E, K435R, R437K, L447M, T449V, L451C, A461T, A463G, T464P, E475D, K477E, G482E, E489Q, R492E, H495K, R496K, L497H, A498K, S499A, S507T, H509Y, Q513T, V523Y, Q524E, R535S, G536D, K538E, L542F, S548T, E560K, and Q568Y;
b) F7Y, E9R, A10P, S13N, S19D, A29T, L40H, L56M, G58K, S62E, P180D, A197K, H201Y, A211K, P220E, R253Q, S279P, A333D, G361N, M364Q, T386K, T399N, A410N, G412A, L451C, A463G, G482E, R496E, L497H, A498K, S499A, V523Y, and G536D;
c) F7Y, E9R, A29T, L40H, G58K, P180D, A197K, H201Y, P220E, R253Q, S279P, G361N, M364Q, T386K, A396E, T399N, A410N, L451C, A463G, G482E, R496E, A498K, and G536D;
d) F7Y, E9R, A10P, S13N, S19D, A29T, L40H, A42G, L56M, G58K, S62E, P180D, A197K, H201Y, A211K, P220E, R253Q, S279D, N328Y, A333D, G361N, M364Q, T386K, R393Q, T399N, A410N, G412A, A434D, T449V, L451C, A463G, G482E, E489Q, R492E, R496K, L497H, A498K, S499A, L516I, V523Y, R535S, and G536D;
e) F7Y, E9R, A10P, S13N, S19D, A29T, L40H, L56M, G58K, S62E, P180D, A197K, H201Y, A211K, P220E, R253Q, A333D, G361N, M364Q, T386K, A396E, T399N, A410N, G412A, L451C, A463G, G482E, R496E, L497H, A498K, S499A, V523Y, and G536D;
f) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, L210F, A211Q, P220E, D227E, R253Q, Q257K, S276K, S279D, R280E, V288K, A292H, F313Y, N328Y, A333D, G361R, M364Q, S379A, T386K, H390T, R393Q, T399N, A410N, G412A, A434D, K435R, R437K, L447M, T449V, L451C, A461T, A463G, E475D, K477E, G482E, E489Q, R492Q, H495K, R496E, L497H, A498K, S499A, S507T, L516I, V523Y, Q524E, R535S, and G536D;
g) F7Y, E9R, A29T, L40H, L56M, G58K, S62E, P180D, A197K, H201Y, P220E, R253Q, S279P, G361N, M364Q, T386K, T399N, A410N, L451C, A463G, G482E, R496E, A498K, S499A, V523Y, and G536D;
h) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, A211Q, P220E, D227E, R253Q, Q257K, S276K, S279P, R280E, V288K, A292H, F313Y, N328Y, A333D, G361N, M364Q, S379A, T386K, H390T, R393Q, T399N, A410N, G412A, A434D, K435R, R437K, L447M, T449V, L451C, A461T, A463G, E475D, K477E, G482D, E489Q, R492E, H495K, R496K, L497H, A498K, S499A, S507T, L516I, V523Y, Q524E, R535S, and G536D;
i) F7Y, E9R, A10P, S13N, S19D, A29T, L40H, A42G, A55E, L56M, G58K, S62E, P180D, E183K, A197K, H201N, L210F, A211Q, P220E, R253Q, S276K, S279P, A292H, N328Y, A333D, G361N, M364Q, T386K, H390N, R393Q, T399N, A410N, G412A, A434D, T449V, L451C, A461E, A463G, G482E, E489Q, H495K, R496E, L497H, A498K, S499A, L516I, V523Y, Q524E, R535S, and G536D;
j) F7Y, E9R, A10P, S13N, S19D, A29T, L40H, A42G, A55E, L56M, G58K, S62E, P180D, E183K, A197K, H201N, A211K, P220E, R253Q, S276K, S279D, A292H, N328Y, A333D, G361N, M364Q, T386K, H390N, R393Q, T399N, A410N, G412A, A434D, T449V, L451C, A461E, A463G, G482E, E489Q, R492E, H495K, R496K, L497H, A498K, S499A, L516I, V523Y, Q524E, R535S, and G536D;
k) F7Y, E9R, A29T, L40H, G58K, P180D, A197K, H201Y, P220E, R253Q, G361N, M364Q, T386K, T399N, A410N, L451C, A463G, G482E, R496E, A498K, and G536D;
l) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, A211Q, P220E, D227E, R253Q, Q257K, S276K, S279P, R280E, V288K, A292H, F313Y, N328Y, A333D, G361N, M364Q, S379A, T386K, H390N, R393Q, T399N, A410N, G412A, A434D, K435R, R437K, L447M, T449V, L451C, A461T, A463G, E475D, K477E, G482E, E489Q, R492E, H495K, R496K, L497H, A498K, S499A, S507T, L516I, V523Y, Q524E, R535S, and G536D;
m) L2N, F7Y, E9R, A10P, S13N, S19D, T21K, L27I, A29T, V35K, L40H, A42G, A55E, L56M, G58K, S62E, S76P, T87E, A93E, A103K, R178K, P180D, E183K, A197K, H201N, A211Q, P220E, D227E, R253Q, Q257K, S276K, S279P, R280E, V288K, A292H, F313Y, A333D, G361N, M364Q, S379A, T386K, H390T, R393Q, T399N, A410N, G412A, A434D, K435R, R437K, L447M, T449V, L451C, A461T, A463G, E475D, K477E, G482D, E489Q, R492E, H495K, R496K, L497H, A498K, S499A, S507T, L516I, V523Y, Q524E, R535S, and G536D;
n) L2N, F7Y, E9R, A10P, S13N, S19D, T21N, Q22H, R24V, L27I, A29T, V35K, L40H, A42G, E50Q, A55E, L56M, G58K, S62E, L72E, S76P, T87E, A93R, A103K, A157S, R178K, P180D, E183K, E184D, A197K, H201N, A211Q, P220E, D227E, R253Q, Q257K, S276K, S279D, R280E, V288K, Q289H, A292H, F313Y, N328Y, A333D, R340K, S344E, G361P, M364Q, S379A, T386K, E388K, H390T, R393Q, A396H, T399N, A410N, G412A, A434E, K435R, R437K, L447M, T449V, L451C, A461T, A463G, T464P, E475D, K477E, G482D, E489Q, R492Q, H495K, R496E, L497H, A498K, S499A, S507T, H509Y, Q513T, L516I, V523Y, Q524E, R535S, G536D, K538E, L542F, S548T, E560K, and Q568Y;
o) F7Y, E9R, A10P, S13N, S19D, A29T, L40H, A42G, L56M, G58K, S62E, P180D, A197K, H201Y, A211E, P220E, R253Q, S279D, N328Y, A333D, G361R, M364Q, T386K, R393Q, T399N, A410N, G412A, A434D, T449V, L451C, A463G, G482E, E489Q, R492E, R496K, L497H, A498K, S499A, L516I, V523Y, R535S, and G536D; or
p) F7Y, E9R, A29T, L40H, L56M, G58K, S62E, P180D, A197K, H201Y, P220E, R253Q, G361N, M364Q, T386K, A396E, T399N, A410N, L451C, A463G, G482E, R496E, A498K, S499A, V523Y, and G536D.

In an embodiment, the present invention provides a nucleic acid sequence encoding the above-described alpha-amylase variant of any one of SEQ ID NO: 1, SEQ ID NO: 2 or SEQ ID NO: 17.

In another embodiment of the present invention, an expression vector comprises the nucleic acid sequence encoding the alpha-amylase variant of any one of SEQ ID NO: 1, SEQ ID NO: 2 or SEQ ID NO: 17.

In an embodiment, the present invention provides a host cell transformed with the expression vector comprising the nucleic acid sequence encoding the alpha-amylase variant of any one of SEQ ID NO: 1, SEQ ID NO: 2 or SEQ ID NO: 17.

In a further embodiment, the present invention provides a method for producing an alpha-amylase variant, said method comprising expressing the alpha-amylase variant in the transformed host cell, wherein the host cell is transformed with the expression vector comprising the nucleic acid sequence encoding the alpha-amylase variant of any one of SEQ ID NO: 1, SEQ ID NO: 2 or SEQ ID NO: 17.

In an embodiment, the present invention provides a method for aiding or supporting digestion of starch in a subject having a deficiency of salivary amylase and/or pancreatic amylase, said method comprising administering to the subject a pharmaceutical composition comprising the alpha-amylase variant of any one of SEQ ID NO:1, SEQ ID NO: 2 or SEQ ID NO: 17.

In another embodiment, the present invention provides a method for brewing beer or liquor, said method comprising fermenting a mash of grains or starch source with the alpha-amylase variant of any one of SEQ ID NO:1, SEQ ID NO: 2 or SEQ ID NO: 17 to produce an alpha amylase variant-digested mixture wherein the fermenting occurs at a temperature of between 70° F. and 90° F.

In an embodiment of the method, the starch source is barley or potato.

In an embodiment of the method, the method further comprises adding to the alpha-amylase variant-digested mixture a beta-amylase variant of a wild-type beta amylase from barley of SEQ ID NO: 3 comprising mutations:
a) Y9H, R26G, G30R, V41K, A64W, N102Y, T127R, I130F, S147T, T157H, V171L, I205Q, N231D, L253Q, N266G, K270Q, D277Q, Y286C, I292A, K302G, R328A, K331A, R334H, M354L, G368A, P386R, T391Q, R394L, L411M, N421D, D437N, P444D, S463P, and P473L;
b) Y9H, G30R, V41K, A64W, N102Y, T127R, I130F, S147T, L167F, V171L, N231D, N266G, K270Q, D277Q, Y286C, I292A, K302G, R328A, K331A, R334H, M354L, G368A, P386S, T391Q, R394L, L411M, N421D, E425H, D437N, P444D, S463P, and P473L;
c) Y9H, R26G, G30R, V41K, A64W, N102Y, I105L, T127R, I130F, S147T, T157H, V171L, V177I, I205Q, F215W, N231D, L253Q, K270Q, D277Q, Y286C, I292A, K302G, R328A, K331A, R334H, Q352E, M354L, S355C, G368A, P386S, T391Q, R394L, L411M, N421D, D437N, P444D, R445H, S463P, and P473L;
d) Y9H, Y13F, A20S, R26G, G30R, A35N, V41A, A64K, A69G, E75Q, L76M, N102Y, I105L, D111E, V112E, G113M, H125W, T127R, I130F, T134S, Q140L, F143L, S147T, A153Q, Y155F, T157H, K164R, L167F, V171L, V177I, A182C, S189F, Q192E, F198Y, I205Q, A213K, F215W, K216M, A221K, V222A, N231D, R242S, D248S, L253Q, A262Q, N266D, K270Q, D277S, V282I, Y286C, A291S, I292A, K302G, V303T, T325A, R328A, K331A, R334H, A335C, S336V, S351Q, Q352E, M354L, S355C, L365A, G368C, P386S, T391Q, R394L, I401L, H409L, L411M, F412Y, N421D, Q422E, E425Q, Q427S, V430R, T434E, D437N, H440S, L443N, P444D, R445H, Y448K, P456N, I462M, S463P, M466D, I467A, P473L, and E482S;
e) Y9H, A20S, R26G, G30R, A35N, V41A, A64K, A69G, E75Q, L76M, N102Y, I105L, D111E, V112E, G113M, H125W, T127R, I130F, T134S, Q140L, S147T, A153Q, Y155F, T157H, K164R, L167F, V171L, V177I, A182C, S189F, Q192E, F198Y, I205Q, A213K, F215W, K216E, A221K, V222A, N231D, R242S, D248S, L253Q, A262Q, N266D, K270Q, D277S, K281A, V282I, Y286C, A291S, I292A, K302G, V303T, T325A, R328A, K331A, R334N, A335C, S336V, S351Q, Q352E, M354L, S355C, L365A, G368C, P386S, T391Q, R394L, I401L, H409L, L411M, F412Y, N421D, Q422E, E425Q, Q427S, V430R, T434E, D437N, H440S, L443N, P444D, R445H, Y448K, P456N, I462M, S463P, M466D, I467A, P473L, and E482S;
f) Y9H, Y13F, R26G, G30R, A35N, V41A, A64K, A69Y, L76M, N102Y, I105L, D111E, V112E, G113M, T127R, I130F, S147T, Y155F, T157H, K164R, V171L, V177I, S189F, F198Y, I205Q, A213K, F215W, K216M, V222A, N231D, R242S, D248S, L253Q, A262Q, N266D, K270Q, D277S, K281A, V282I, Y286C, A291S, I292A, K302G, T325A, R328A, K331A, R334N, A335C, S336V, S351P, Q352E, M354L, S355C, Q362R, L365A, G368C, P386S, T391Q, R394L, H409L, L411M, N421D, Q422E, Q427S, V430M, T434E, D437K, H440S, L443N, P444D, R445H, Y448K, P456N, I462M, S463P, M466D, I467E, and P473L;
g) Y9H, A20T, G30R, V41K, A64W, N102Y, T127R, I130F, S147T, L167F, V171L, N231D, N266G, K270Q, Y286C, I292V, K302G, R328A, K331A, R334H, M354L, G368A, P386S, T391Q, R394L, L411M, N421D, D437N, P444D, S463P, and P473L;
h) Y13F, R26G, G30R, A35N, V41A, A64K, A69G, L76M, N102Y, I105L, D111E, V112E, G113M, T127R, I130F, S147T, Y155F, T157H, K164R, L167F, V171L, V177I, S189F, F198Y, I205Q, A213K, F215W, K216M, V222A, N231D, R242S, D248S, L253Q, A262Q, N266D, K270Q, D277S, K281S, V282I, Y286C, A291S, I292A, K302G, T325A, R328A, K331A, R334H, A335C, S336V, S351P, Q352E, M354L, S355C, Q362R, L365A, G368C, P386R, T391Q, R394L, H409L, L411M, N421D, Q422E, E425Q, Q427S, V430W, T434E, D437N, H440S, L443N, P444D, R445H, Y448K, P456N, I462M, S463P, M466D, I467E, and P473L;
i) L16M, R26G, G30R, V41A, E42N, A64K, L76M, N102Y, I105L, D111E, T127R, I130F, S147T, Y155F, T157H, L167F, V171L, V177I, S189F, I205Q, A213K, F215W, K216M, V222A, N231D, R242S, L253Q, N266G, K270Q, K281S, Y286C, I292V, K302G, R328A, K331A, R334H, A335C, S336V, S351P, Q352E, M354L, S355C, Q362R, L365M, G368A, P386R, T391Q, R394L, H409L, L411M, N421D, Q422E, E425H, Q427S, D437N, H440S, L443N, P444D, R445H, Y448K, P456N, I462M, S463P, and P473L;
j) R26G, G30R, V41A, A64K, L76M, N102Y, I105L, D111E, T127R, I130F, S147T, Y155F, T157R, L167F, V171L, V177I, S189F, I205Q, A213K, F215W, K216M, V222A, N231D, R242S, L253Q, N266G, K270Q, D277Q, K281S, Y286C, I292A, K302G, R328A, K331A, R334N, A335C, S336T, S351P, Q352E, M354L, S355C, Q362R, L365M, G368A, P386S, T391Q, R394L, H409L, L411M, N421D, Q422E, E425H, Q427S, D437N, H440S, L443N, P444D, R445H, Y448K, P456N, I462M, S463P, and P473L;
k) Y9H, R26G, G30R, V41K, A64W, N102F, I105L, T127R, I130F, S147T, T157H, L167F, V171L, V177I, I205Q, F215W, N231D, L253Q, N266G, K270Q, D277Q, Y286C, I292A, K302G, R328A, K331A, R334H, Q352E, M354L, S355C, G368A, P386S, T391Q, R394L, L411M, N421D, D437N, P444D, R445H, S463P, and P473L;
l) Y9H, R26G, G30R, V41R, A64W, N102Y, I105L, T127R, I130F, S147T, T157H, V171L, V177I, I205Q, F215W, V222A, N231D, R242S, L253Q, N266G, K270Q, Y286C, I292A, K302G, R328A, K331A, R334H, A335C, S336V, Q352E, M354L, S355C, G368A, N374Q, P386S, T391Q, R394L, L411M, N421D, E425H, D437N, H440S, P444D, R445H, P456N, S463P, and P473L;
m) Y9H, R26G, G30R, V41K, A64W, N102Y, T127R, I130F, S147T, T157H, V171L, I205Q, N231D, L253Q, N266G, K270Q, D277Q, Y286C, I292A, K302G, R328A, K331A, R334H, M354L, G368A, P386S, T391Q, R394L, L411M, N421D, E425H, D437N, P444D, S463P, and P473L; or
n) Y9H, R26G, G30R, V41A, A64W, N102Y, I105L, T127R, I130F, S147T, T157H, L167F, V171L, V177I, I205Q, F215W, V222A, N231D, R242S, L253Q, N266G, K270Q, D277Q, Y286C, I292A, K302G, R328A, K331A, R334H, A335C, Q352E, M354L, S355C, G368A, N374Q, P386S, T391Q, R394L, L411M, N421D, E425H, D437N, H440S, P444D, R445H, P456N, S463P, and P473L.

In another embodiment of the method, the method further comprises adding to the alpha-amylase variant-digested mixture a beta-amylase variant of a wild-type beta amylase from sweet potato of SEQ ID NO: 4 comprising mutations:
a) I4T, V7S, D39A, K42H, V44L, G47A, G48N, C49V, I60V, A63K, K67M, A73Y, R75H, K82R, K87H, I88L, A90V, V105H, F106T, A129S, L137I, V141C, L146V, E161R, A173D, D175L, T197N, V201A, V216Q, E249T, K272S, I274E, E282A, L291C, R292G, N294S, A296S, K298R, V309R, F319Y, V322T, G324N, P330A, A332L, T341I, E357N, Q363E, E364Q, S371N, S372A, G373A, Y377D, D379H, T392A, L408R, K439A, S451C, A454V, and D486N;
b) I4T, V7S, K42H, V44L, K45A, G47A, G48N, C49V, I60V, A63K, K67Q, R75H, K87H, I88L, A90V, V105F, F106T, A129S, L137I, E161R, A173D, D175L, T197N, V201A, V216R, E249T, K272S, E282A, V287I, L291T, N294S, A296S, K298R, V309R, V322T, G324N, P330A, T341I, E357N, Q363E, E364Q, S371N, G373A, Y377G, T392A, L408R, T431E, K439A, S451N, A454V, and D486N;
c) D486N;
d) I4T, V7S, G23D, V30N, D39A, K42M, V44L, K45A, G47A, G48N, C49V, I60N, K67M, A73G, R75H, L80M, K87H, I88L, A90V, F106T, I113V, Q115E, A129S, L137I, V141C, L146V, E161Q, A173D, D175L, E196Q, T197N, V201A, M215L, V216K, E221N, V223A, Q225K, M233S, G235K, E249T, N271Q, K272C, I274E, I275Q, G277L, E282A, L291C, R292G, N294T, I295L, A296S, K298R, S300A, N307K, V309R, F319Y, V322T, G324N, P330A, E357N, Q363E, E364G, S371N, S372A, G373A, Y377G, D379H, T392A, L398M, L408R, N409E, T431N, D432Q, K439A, P450H, S451C, N453E, A454V, S456D, and D486N;
e) I4T, V7S, D39A, K42Q, V44L, K45A, G47A, G48N, C49V, I60V, K67Q, A73Y, R75H, K87H, I88L, A90V, V105F, F106T, A129S, L137I, V141C, E161R, A173D, D175L, T197N, V201A, V216Q, E249T, K272S, I274E, E282A, L291C, R292G, N294T, A296S, K298R, V309R, F319Y, V322T, G324N, P330A, E357N, Q363E, E364A, S371N, S372A, G373A, Y377G, D379H, T392A, L408R, T431E, K439A, S451N, A454V, and D486N;
f) I4T, V7S, G23D, V30N, D39W, K42M, V44L, G47A, G48N, C49E, I60V, A63K, K67Q, A73G, R75H, K87H, I88L, A90V, V105H, F106T, I113V, Q115E, A129S, V141C, L146V, E161R, A173D, D175L, T197N, V201A, V216Q, E221N, G235K, E249T, T267Q, K272S, I274E, I275H, Q279R, E282A, L291C, R292G, N294T, A296S, K298R, V309R, F319Y, V322T, G324N, P330A, E357N, Q363E, E364Q, S371N, S372A, G373A, Y377G, D379H, T392A, L408R, N409E, T431E, D432S, K439A, P450H, S451C, N453E, A454V, S456D, and D486N;
g) I4T, V7S, K42H, V44L, K45A, G47A, G48N, C49V, I60V, K67Q, K87H, I88L, A90V, V105F, A129S, L137I, L146V, E161R, A173D, D175L, T197N, V201A, V216R, E249T, K272C, I275Q, E282A, L291T, N294T, I295L, A296S, K298R, V309R, V322T, G324N, P330A, T341I, E357N, Q363E, E364Q, S371N, G373A, Y377D, T392Q, L408R, T431E, K439A, S451C, A454V, A458P, and D486N;
h) I4T, V7S, K42H, V44L, K45A, G47A, G48N, C49V, I60V, A63K, K67Q, K87H, I88L, A90V, V105C, A129S, L137I, L146V, E161R, A173D, D175L, T197N, V201A, V216R, E249T, K272S, E282A, L291T, N294S, A296S, K298R, V309R, V322T, G324N, P330A, T341I, E357N, Q363E, E364Q, S371N, G373A, Y377G, T392Q, L408K, T431E, K439A, S451N, A454V, and D486N;
i) I4T, V7S, G23D, A27R, V30N, V37C, D39Q, K42M, V44L, K45A, G47A, G48N, C49E, I60N, I61V, A63S, K67Q, A73G, R75H, L80M, V81C, K87H, I88L, A90V, V105H, F106T, I113V, Q115E, N127D, A129S, V141C, L146V, E161R, A173D, D175L, E196Q, T197N, V201A, M215L, V216K, E221N, V223A, Q225K, M233L, G235K, E249T, R252K, N271Q, K272S, I274E, I275Q, G277L, V280I, E282A, L291C, R292G, N294T, I295L, A296S, K298R, S300A, N307K, H308T, V309R, F319Y, V322T, A323R, G324N, P330A, A332L, E357N, Q363E, E364Q, S371N, S372A, G373A, K375D, Y377G, D379H, T392A, L408R, N409E, Q430E, T431N, D432N, K439A, K442D, P450H, S451C, N453E, A454V, S456D, N464K, and D486N;
j) I4T, V7S, K42E, V44L, G47A, G48N, C49V, M53V, I60V, K67Q, R75K, K87H, I88L, A90V, V105F, F106T, A129S, L137I, E161R, D175L, T197N, V201A, V216R, E249T, D260E, T267Q, K272S, E282A, L291T, N294T, A296S, K298R, V309R, V322T, G324N, P330A, E357N, Q363E, E364Q, S371N, G373A, Y377G, T392A, L408R, T431E, K439A, S451C, A454V, and D486N;
k) I4T, V7S, G23D, V30N, D39W, K42M, V44L, K45A, G47A, G48N, C49E, I60N, A63K, K67Q, A73Y, R75H, L80M, K87H, I88L, A90V, F106T, I113V, Q115E, A129S, L137I, V141C, L146V, E161Q, A173D, D175L, E196Q, T197N, V201A, M215L, V216K, E221N, V223A, Q225K, M233S, G235K, E249T, K272V, I274E, G277L, E282A, L291C, R292G, N294T, A296S, K298R, S300A, N307K, V309R, F319Y, V322T, G324N, P330A, E357N, Q363E, E364G, S371N, S372A, G373A, Y377G, D379H, T392A, L398M, L408K, N409E, T431N, D432Q, F434Y, K439A, P450H, S451C, N453E, A454V, S456D, and D486N;
l) I4T, V7S, V30N, D39A, K42Q, V44L, G47A, G48N, C49V, I60V, A63K, K67Q, A73Y, I88L, A90V, V105F, F106T, I113V, A129S, L137I, V141C, L146V, D165N, A173D, D175L, T197N, V201S, V216K, E221N, E249T, K272S, I274E, E282A, L291C, R292G, N294T, A296S, K298R, V309R, F319Y, V322T, G324N, P330A, E357N, Q363E, E364Q, S371N, S372A, G373A, Y377G, D379H, T392A, L408R, T431E, F434Y, K439A, P450H, S451C, A454V, S456D, and D486N;
m) I4T, V7S, V30N, D39W, K42M, V44L, G47A, G48N, C49V, I60V, K67Q, A73Y, R75H, I88L, A90V, V105C, F106T, I113V, A129S, V141C, L146V, E161R, A173D, D175L, T197N, V201A, V216K, E221N, E249T, K272S, I274E, E282A, L291C, R292G, N294S, A296S, K298R, V309R, F319Y, V322T, G324N, P330A, T341I, E357N, Q363E, E364Q, S371N, S372A, G373A, Y377D, D379H, T392A, L408R, K439N, P450H, S451C, A454V, S456D, and D486N;
n) I4T, V7S, G23D, A27R, V30N, V37C, D39Q, K42M, V44L, G47A, G48N, C49E, I60N, I61V, A63S, K67Q, A73Y, L80M, V81C, K87H, I88L, A90V, V105H, F106S, I113V, Q115E, N127D, A129S, L137I, V141C, L146V, E161R, D175L, E196Q, T197N, V201A, M215L, V216K, E221N, V223A, Q225K, M233L, G235K, E249T, R252K, N271Q, K272S, I274E, I275Q, G277L, V280I, E282A, L291C, R292G, N294A, I295L, A296S, K298R, S300A, N307K, H308T, V309R, F319Y, V322T, A323R, G324N, P330A, A332L, T341I, E357N, Q363E, E364G, S371N, S372A, G373A, K375D, Y377G, D379H, T392A, L398M, L408R, N409E, Q430E, T431N, D432Q, L436I, K439A, K442D, P450H, S451C, N453E, A454V, S456D, N464K, and D486N; or
o) I4T, V7S, G23D, V30N, D39W, K42Q, V44L, K45A, G47N, G48N, C49E, I60V, K67R, A73Y, K87H, I88L, A90V, V105F, F106T, I113V, Q115E, A129S, L137I, V141C, L146V, E161Q, A173D, D175L, T197N, V201A, V216K, E221N, G235K, E249T, K272S, I274E, E282A, L291C, R292K, N294S, A296S, K298R, V309R, F319Y, V322T, G324N, P330A, T341I, E357N, Q363E, E364A, S371N, S372A, G373A, Y377D, D379H, T392A, L408R, N409E, T431E, D432Q, K439A, P450H, S451C, N453E, A454V, S456D, and D486N.

In a further embodiment, the present invention provides a detergent or stain-removing spotting concentrate comprising the any of the above-described alpha-amylase variant of any one of SEQ ID NO: 1, SEQ ID NO: 2 or SEQ ID NO: 17.

In another embodiment, the present invention provides a composition for desizing a cotton fabric comprising any of the above-described alpha-amylase variant of any one of SEQ ID NO: 1, SEQ ID NO: 2 or SEQ ID NO: 17, wherein desizing occurs at a temperature of between 30° F. and 60° F.

In yet another embodiment, the present invention provides an animal feed additive comprising the above-described alpha-amylase variant of any one of SEQ ID NO: 1, SEQ ID NO: 2 or SEQ ID NO: 17.

In an embodiment, the present invention provides a method for baking a food product, said method comprising adding to flour or a flour-containing dough above-described alpha-amylase variant of any one of SEQ ID NO: 1, SEQ ID NO: 2 or SEQ ID NO: 17.

In an embodiment of the method, addition of the alpha-amylase variant to the flour or the flour-containing dough enhances rate of fermentation of the flour-containing dough and/or reduces viscosity of the flour-containing dough.

In another embodiment of the method, wherein addition of the alpha-amylase variant to the flour or the flour-containing dough improves taste of the food product by generating sugar in the flour-containing dough and/or increases shelf-life of the food product compared to a food product baked with flour or a flour-containing dough which does not comprise the alpha-amylase variant.

### Beta-Amylase Variants

Beta-amylase is an enzyme which hydrolyzes starch, glycogen and relates polysaccharides and oligosaccharides. The optimum pH for beta-amylase is 4.0-5.0. Beta-amylase is found in plants, seeds, bacteria and fungi, but not in animals, except possibly in microorganisms in the digestive tract. Beta-amylase breaks starch into maltose during fruit ripening, resulting in the sweet flavor of ripe fruit.

Beta-amylase has several industrial applications. As with alpha-amylase, beta-amylase is used for fermentation in brewing alcohol and distilling, such as beer and liquor, from starch sources, such as barley and potatoes, to release the disaccharide maltose, the single product of beta-amylase hydrolysis of starch. In brewing, beta-amylase has an optimum pH of 5.2 and an optimum temperature of about 60° C. Beta-amylase also is used in production of maltose syrup and as an additive in baking, e.g., of bread and other baked products.

The beta-amylase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type beta-amylases will denature, decompose and/or under which the beta-amylase enzymatic function is significantly reduced or eliminated.

The present invention provides a beta-amylase variant of a wild-type beta amylase from barley of SEQ ID NO: 3 comprising mutations:
a) Y9H, R26G, G30R, V41K, A64W, N102Y, T127R, I130F, S147T, T157H, V171L, I205Q, N231D, L253Q, N266G, K270Q, D277Q, Y286C, I292A, K302G, R328A, K331A, R334H, M354L, G368A, P386R, T391Q, R394L, L411M, N421D, D437N, P444D, S463P, and P473L;
b) Y9H, G30R, V41K, A64W, N102Y, T127R, I130F, S147T, L167F, V171L, N231D, N266G, K270Q, D277Q, Y286C, I292A, K302G, R328A, K331A, R334H, M354L, G368A, P386S, T391Q, R394L, L411M, N421D, E425H, D437N, P444D, S463P, and P473L;
c) Y9H, R26G, G30R, V41K, A64W, N102Y, I105L, T127R, I130F, S147T, T157H, V171L, V177I, I205Q, F215W, N231D, L253Q, K270Q, D277Q, Y286C, I292A, K302G, R328A, K331A, R334H, Q352E, M354L, S355C, G368A, P386S, T391Q, R394L, L411M, N421D, D437N, P444D, R445H, S463P, and P473L;
d) Y9H, Y13F, A20S, R26G, G30R, A35N, V41A, A64K, A69G, E75Q, L76M, N102Y, I105L, D111E, V112E, G113M, H125W, T127R, I130F, T134S, Q140L, F143L, S147T, A153Q, Y155F, T157H, K164R, L167F, V171L, V177I, A182C, S189F, Q192E, F198Y, I205Q, A213K, F215W, K216M, A221K, V222A, N231D, R242S, D248S, L253Q, A262Q, N266D, K270Q, D277S, V282I, Y286C, A291S, I292A, K302G, V303T, T325A, R328A, K331A, R334H, A335C, S336V, S351Q, Q352E, M354L, S355C, L365A, G368C, P386S, T391Q, R394L, I401L, H409L, L411M, F412Y, N421D, Q422E, E425Q, Q427S, V430R, T434E, D437N, H440S, L443N, P444D, R445H, Y448K, P456N, I462M, S463P, M466D, I467A, P473L, and E482S;
e) Y9H, A20S, R26G, G30R, A35N, V41A, A64K, A69G, E75Q, L76M, N102Y, I105L, D111E, V112E, G113M, H125W, T127R, I130F, T134S, Q140L, S147T, A153Q, Y155F, T157H, K164R, L167F, V171L, V177I, A182C, S189F, Q192E, F198Y, I205Q, A213K, F215W, K216E, A221K, V222A, N231D, R242S, D248S, L253Q, A262Q, N266D, K270Q, D277S, K281A, V282I, Y286C, A291S, I292A, K302G, V303T, T325A, R328A, K331A, R334N, A335C, S336V, S351Q, Q352E, M354L, S355C, L365A, G368C, P386S, T391Q, R394L, I401L, H409L, L411M, F412Y, N421D, Q422E, E425Q, Q427S, V430R, T434E, D437N, H440S, L443N, P444D, R445H, Y448K, P456N, I462M, S463P, M466D, I467A, P473L, and E482S;
f) Y9H, Y13F, R26G, G30R, A35N, V41A, A64K, A69Y, L76M, N102Y, I105L, D111E, V112E, G113M, T127R, I130F, S147T, Y155F, T157H, K164R, V171L, V177I, S189F, F198Y, I205Q, A213K, F215W, K216M, V222A, N231D, R242S, D248S, L253Q, A262Q, N266D, K270Q, D277S, K281A, V282I, Y286C, A291S, I292A, K302G, T325A, R328A, K331A, R334N, A335C, S336V, S351P, Q352E, M354L, S355C, Q362R, L365A, G368C, P386S, T391Q, R394L, H409L, L411M, N421D, Q422E, Q427S, V430M, T434E, D437K, H440S, L443N, P444D, R445H, Y448K, P456N, I462M, S463P, M466D, I467E, and P473L;
g) Y9H, A20T, G30R, V41K, A64W, N102Y, T127R, I130F, S147T, L167F, V171L, N231D, N266G, K270Q, Y286C, I292V, K302G, R328A, K331A, R334H, M354L, G368A, P386S, T391Q, R394L, L411M, N421D, D437N, P444D, S463P, and P473L;
h) Y13F, R26G, G30R, A35N, V41A, A64K, A69G, L76M, N102Y, I105L, D111E, V112E, G113M, T127R, I130F, S147T, Y155F, T157H, K164R, L167F, V171L, V177I, S189F, F198Y, I205Q, A213K, F215W, K216M, V222A, N231D, R242S, D248S, L253Q, A262Q, N266D, K270Q, D277S, K281S, V282I, Y286C, A291S, I292A, K302G, T325A, R328A, K331A, R334H, A335C, S336V, S351P, Q352E, M354L, S355C, Q362R, L365A, G368C, P386R, T391Q, R394L, H409L, L411M, N421D, Q422E, E425Q, Q427S, V430W, T434E, D437N, H440S, L443N, P444D, R445H, Y448K, P456N, I462M, S463P, M466D, I467E, and P473L;
i) L16M, R26G, G30R, V41A, E42N, A64K, L76M, N102Y, I105L, D111E, T127R, I130F, S147T, Y155F, T157H, L167F, V171L, V177I, S189F, I205Q, A213K, F215W, K216M, V222A, N231D, R242S, L253Q, N266G, K270Q, K281S, Y286C, I292V, K302G, R328A, K331A, R334H, A335C, S336V, S351P, Q352E, M354L, S355C, Q362R, L365M, G368A, P386R, T391Q, R394L, H409L, L411M, N421D, Q422E, E425H, Q427S, D437N, H440S, L443N, P444D, R445H, Y448K, P456N, I462M, S463P, and P473L;
j) R26G, G30R, V41A, A64K, L76M, N102Y, I105L, D111E, T127R, I130F, S147T, Y155F, T157R, L167F, V171L, V177I, S189F, I205Q, A213K, F215W, K216M, V222A, N231D, R242S, L253Q, N266G, K270Q, D277Q, K281S, Y286C, I292A, K302G, R328A, K331A, R334N, A335C, S336T, S351P, Q352E, M354L, S355C, Q362R, L365M, G368A, P386S, T391Q, R394L, H409L, L411M, N421D, Q422E, E425H, Q427S, D437N, H440S, L443N, P444D, R445H, Y448K, P456N, I462M, S463P, and P473L;
k) Y9H, R26G, G30R, V41K, A64W, N102F, I105L, T127R, I130F, S147T, T157H, L167F, V171L, V177I, I205Q, F215W, N231D, L253Q, N266G, K270Q, D277Q, Y286C, I292A, K302G, R328A, K331A, R334H, Q352E, M354L, S355C, G368A, P386S, T391Q, R394L, L411M, N421D, D437N, P444D, R445H, S463P, and P473L;
l) Y9H, R26G, G30R, V41R, A64W, N102Y, I105L, T127R, I130F, S147T, T157H, V171L, V177I, I205Q, F215W, V222A, N231D, R242S, L253Q, N266G, K270Q, Y286C, I292A, K302G, R328A, K331A, R334H, A335C, S336V, Q352E, M354L, S355C, G368A, N374Q, P386S, T391Q, R394L, L411M, N421D, E425H, D437N, H440S, P444D, R445H, P456N, S463P, and P473L;
m) Y9H, R26G, G30R, V41K, A64W, N102Y, T127R, I130F, S147T, T157H, V171L, I205Q, N231D, L253Q, N266G, K270Q, D277Q, Y286C, I292A, K302G, R328A, K331A, R334H, M354L, G368A, P386S, T391Q, R394L, L411M, N421D, E425H, D437N, P444D, S463P, and P473L; or
n) Y9H, R26G, G30R, V41A, A64W, N102Y, I105L, T127R, I130F, S147T, T157H, L167F, V171L, V177I, I205Q, F215W, V222A, N231D, R242S, L253Q, N266G, K270Q, D277Q, Y286C, I292A, K302G, R328A, K331A, R334H, A335C, Q352E, M354L, S355C, G368A, N374Q, P386S, T391Q, R394L, L411M, N421D, E425H, D437N, H440S, P444D, R445H, P456N, S463P, and P473L.

The present invention also provides a beta amylase variant of a wild-type beta amylase from sweet potato of SEQ ID NO: 4 comprising mutations:
a) I4T, V7S, D39A, K42H, V44L, G47A, G48N, C49V, I60V, A63K, K67M, A73Y, R75H, K82R, K87H, I88L, A90V, V105H, F106T, A129S, L137I, V141C, L146V, E161R, A173D, D175L, T197N, V201A, V216Q, E249T, K272S, I274E, E282A, L291C, R292G, N294S, A296S, K298R, V309R, F319Y, V322T, G324N, P330A, A332L, T341I, E357N, Q363E, E364Q, S371N, S372A, G373A, Y377D, D379H, T392A, L408R, K439A, S451C, A454V, and D486N;
b) I4T, V7S, K42H, V44L, K45A, G47A, G48N, C49V, I60V, A63K, K67Q, R75H, K87H, I88L, A90V, V105F, F106T, A129S, L137I, E161R, A173D, D175L, T197N, V201A, V216R, E249T, K272S, E282A, V287I, L291T, N294S, A296S, K298R, V309R, V322T, G324N, P330A, T341I, E357N, Q363E, E364Q, S371N, G373A, Y377G, T392A, L408R, T431E, K439A, S451N, A454V, and D486N;
c) D486N;
d) I4T, V7S, G23D, V30N, D39A, K42M, V44L, K45A, G47A, G48N, C49V, I60N, K67M, A73G, R75H, L80M, K87H, I88L, A90V, F106T, I113V, Q115E, A129S, L137I, V141C, L146V, E161Q, A173D, D175L, E196Q, T197N, V201A, M215L, V216K, E221N, V223A, Q225K, M233S, G235K, E249T, N271Q, K272C, I274E, I275Q, G277L, E282A, L291C, R292G, N294T, I295L, A296S, K298R, S300A, N307K, V309R, F319Y, V322T, G324N, P330A, E357N, Q363E, E364G, S371N, S372A, G373A, Y377G, D379H, T392A, L398M, L408R, N409E, T431N, D432Q, K439A, P450H, S451C, N453E, A454V, S456D, and D486N;
e) I4T, V7S, D39A, K42Q, V44L, K45A, G47A, G48N, C49V, I60V, K67Q, A73Y, R75H, K87H, I88L, A90V, V105F, F106T, A129S, L137I, V141C, E161R, A173D, D175L, T197N, V201A, V216Q, E249T, K272S, I274E, E282A, L291C, R292G, N294T, A296S, K298R, V309R, F319Y, V322T, G324N, P330A, E357N, Q363E, E364A, S371N, S372A, G373A, Y377G, D379H, T392A, L408R, T431E, K439A, S451N, A454V, and D486N;
f) I4T, V7S, G23D, V30N, D39W, K42M, V44L, G47A, G48N, C49E, I60V, A63K, K67Q, A73G, R75H, K87H, I88L, A90V, V105H, F106T, I113V, Q115E, A129S, V141C, L146V, E161R, A173D, D175L, T197N, V201A, V216Q, E221N, G235K, E249T, T267Q, K272S, I274E, I275H, Q279R, E282A, L291C, R292G, N294T, A296S, K298R, V309R, F319Y, V322T, G324N, P330A, E357N, Q363E, E364Q, S371N, S372A, G373A, Y377G, D379H, T392A, L408R, N409E, T431E, D432S, K439A, P450H, S451C, N453E, A454V, S456D, and D486N;
g) I4T, V7S, K42H, V44L, K45A, G47A, G48N, C49V, I60V, K67Q, K87H, I88L, A90V, V105F, A129S, L137I, L146V, E161R, A173D, D175L, T197N, V201A, V216R, E249T, K272C, I275Q, E282A, L291T, N294T, I295L, A296S, K298R, V309R, V322T, G324N, P330A, T341I, E357N, Q363E, E364Q, S371N, G373A, Y377D, T392Q, L408R, T431E, K439A, S451C, A454V, A458P, and D486N;
h) I4T, V7S, K42H, V44L, K45A, G47A, G48N, C49V, I60V, A63K, K67Q, K87H, I88L, A90V, V105C, A129S, L137I, L146V, E161R, A173D, D175L, T197N, V201A, V216R, E249T, K272S, E282A, L291T, N294S, A296S, K298R, V309R, V322T, G324N, P330A, T341I, E357N, Q363E, E364Q, S371N, G373A, Y377G, T392Q, L408K, T431E, K439A, S451N, A454V, and D486N;
i) I4T, V7S, G23D, A27R, V30N, V37C, D39Q, K42M, V44L, K45A, G47A, G48N, C49E, I60N, I61V, A63S, K67Q, A73G, R75H, L80M, V81C, K87H, I88L, A90V, V105H, F106T, I113V, Q115E, N127D, A129S, V141C, L146V, E161R, A173D, D175L, E196Q, T197N, V201A, M215L, V216K, E221N, V223A, Q225K, M233L, G235K, E249T, R252K, N271Q, K272S, I274E, I275Q, G277L, V280I, E282A, L291C, R292G, N294T, I295L, A296S, K298R, S300A, N307K, H308T, V309R, F319Y, V322T, A323R, G324N, P330A, A332L, E357N, Q363E, E364Q, S371N, S372A, G373A, K375D, Y377G, D379H, T392A, L408R, N409E, Q430E, T431N, D432N, K439A, K442D, P450H, S451C, N453E, A454V, S456D, N464K, and D486N;
j) I4T, V7S, K42E, V44L, G47A, G48N, C49V, M53V, I60V, K67Q, R75K, K87H, I88L, A90V, V105F, F106T, A129S, L137I, E161R, D175L, T197N, V201A, V216R, E249T, D260E, T267Q, K272S, E282A, L291T, N294T, A296S, K298R, V309R, V322T, G324N, P330A, E357N, Q363E, E364Q, S371N, G373A, Y377G, T392A, L408R, T431E, K439A, S451C, A454V, and D486N;
k) I4T, V7S, G23D, V30N, D39W, K42M, V44L, K45A, G47A, G48N, C49E, I60N, A63K, K67Q, A73Y, R75H, L80M, K87H, I88L, A90V, F106T, I113V, Q115E, A129S, L137I, V141C, L146V, E161Q, A173D, D175L, E196Q, T197N, V201A, M215L, V216K, E221N, V223A, Q225K, M233S, G235K, E249T, K272V, I274E, G277L, E282A, L291C, R292G, N294T, A296S, K298R, S300A, N307K, V309R, F319Y, V322T, G324N, P330A, E357N, Q363E, E364G, S371N, S372A, G373A, Y377G, D379H, T392A, L398M, L408K, N409E, T431N, D432Q, F434Y, K439A, P450H, S451C, N453E, A454V, S456D, and D486N;
l) I4T, V7S, V30N, D39A, K42Q, V44L, G47A, G48N, C49V, I60V, A63K, K67Q, A73Y, I88L, A90V, V105F, F106T, I113V, A129S, L137I, V141C, L146V, D165N, A173D, D175L, T197N, V201S, V216K, E221N, E249T, K272S, I274E, E282A, L291C, R292G, N294T, A296S, K298R, V309R, F319Y, V322T, G324N, P330A, E357N, Q363E, E364Q, S371N, S372A, G373A, Y377G, D379H, T392A, L408R, T431E, F434Y, K439A, P450H, S451C, A454V, S456D, and D486N;
m) I4T, V7S, V30N, D39W, K42M, V44L, G47A, G48N, C49V, I60V, K67Q, A73Y, R75H, I88L, A90V, V105C, F106T, I113V, A129S, V141C, L146V, E161R, A173D, D175L, T197N, V201A, V216K, E221N, E249T, K272S, I274E, E282A, L291C, R292G, N294S, A296S, K298R, V309R, F319Y, V322T, G324N, P330A, T341I, E357N, Q363E, E364Q, S371N, S372A, G373A, Y377D, D379H, T392A, L408R, K439N, P450H, S451C, A454V, S456D, and D486N;
n) I4T, V7S, G23D, A27R, V30N, V37C, D39Q, K42M, V44L, G47A, G48N, C49E, I60N, I61V, A63S, K67Q, A73Y, L80M, V81C, K87H, I88L, A90V, V105H, F106S, I113V, Q115E, N127D, A129S, L137I, V141C, L146V, E161R, D175L, E196Q, T197N, V201A, M215L, V216K, E221N, V223A, Q225K, M233L, G235K, E249T, R252K, N271Q, K272S, I274E, I275Q, G277L, V280I, E282A, L291C, R292G, N294A, I295L, A296S, K298R, S300A, N307K, H308T, V309R, F319Y, V322T, A323R, G324N, P330A, A332L, T341I, E357N, Q363E, E364G, S371N, S372A, G373A, K375D, Y377G, D379H, T392A, L398M, L408R, N409E, Q430E, T431N, D432Q, L436I, K439A, K442D, P450H, S451C, N453E, A454V, S456D, N464K, and D486N; or
o) I4T, V7S, G23D, V30N, D39W, K42Q, V44L, K45A, G47N, G48N, C49E, I60V, K67R, A73Y, K87H, I88L, A90V, V105F, F106T, I113V, Q115E, A129S, L137I, V141C, L146V, E161Q, A173D, D175L, T197N, V201A, V216K, E221N, G235K, E249T, K272S, I274E, E282A, L291C, R292K, N294S, A296S, K298R, V309R, F319Y, V322T, G324N, P330A, T341I, E357N, Q363E, E364A, S371N, S372A, G373A, Y377D, D379H, T392A, L408R, N409E, T431E, D432Q, K439A, P450H, S451C, N453E, A454V, S456D, and D486N.

In an embodiment, the present invention provides a nucleic acid sequence encoding the above-described beta-amylase variant of any one of SEQ ID NO: 3 or SEQ ID NO: 4.

In another embodiment of the present invention, an expression vector comprises the nucleic acid sequence encoding the beta-amylase variant of any one of SEQ ID NO: 3 or SEQ ID NO: 4.

In an embodiment, the present invention provides a host cell transformed with the expression vector comprising the nucleic acid sequence encoding the beta-amylase variant of any one of SEQ ID NO: 3 or SEQ ID NO: 4.

In a further embodiment, the present invention provides a method for producing a beta-amylase variant, said method comprising expressing the beta-amylase variant in the transformed host cell, wherein the host cell is transformed with the expression vector comprising the nucleic acid sequence encoding the beta-amylase variant of any one of SEQ ID NO: 3 or SEQ ID NO: 4.

In an embodiment, the present invention provides a method for brewing beer or liquor, said method comprising fermenting a mash of grains or starch source with the beta-amylase variant of any one of SEQ ID NO: 3 or SEQ ID NO: 4, wherein the fermenting occurs at a temperature of between 70° F. and 90° F.

In further embodiments of any of the herein provided methods for brewing beer or liquor, the starch source is barley or potato.

### Cellulase Variants

Cellulases are enzymes that break down cellulose into monosaccharides, such as beta-glucose, or shorter polysaccharides and oligosaccharides. Cellulases also hydrolyze cellulose and related polysaccharide material. Cellulases are produced by bacteria, fungi, protozoans.

Cellulases are used in various industries, for example, to hydrolyse cellulose during drying of coffee beans, in the pulp and paper industry, in the textile industry, as additives in laundry detergents, in the pharmaceutical industry, e.g., to reduce phytobezoar, which is indigestible plant material, such as fiber, skin and seeds, that is trapped in the gastrointestinal system, especially the stomach, in production of biofuel from biomass, and in degradation of bacterial biofilms. Ruminants, such as cows, sheep, deer, and goats, have microbes in their foregut, i.e., the rumen, that break down cellulose. Humans lack the cellulase enzyme to digest cellulose, and therefore, cannot digest cellulose found in plant food and other dietary fiber.

The cellulase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type cellulase will denature, decompose and/or under which the beta-amylase enzymatic function is significantly reduced or eliminated.

The present invention provides a cellulase variant of a wild-type cellulose from Trichoderma reesei of SEQ ID NO: 5 comprising mutations L81C, P83Y, D84G, E85N, A86D, T87Y, G89S, K90Q, and N91C.

The present invention also provides a cellulase variant of a wild-type cellulase from Trichoderma reesei of SEQ ID NO: 18 comprising mutations:
a) T42C, V48C, T72C, P83C, G89C, K90R, Y93C, S125N, K144Q, G162C, L169F, D173E, N175S, A178K, D194C, P198C, S210K, N217C, L222W, T237C, D242C, S243A, G247C, S253K, S283G, T292H, R296E, P316C, A325G, K329S, I341M, R356N, S361C, A372Q, G376D, and T393S;
b) T42C, V48C, T72C, P83C, G89C, Y93C, E139N, G162C, N175S, N179S, N187A, D194C, P198C, N217C, T237C, D242C, G247C, S283G, Q297R, P316C, A325G, I341L, R356N, A357N, S361C, G376N, V380T, and T393S;
c) T42C, V48C, T72C, P83C, G89C, Y93C, E139N, G162C, D173E, N187A, D194C, P198C, N217C, T237C, D242C, G247C, S283G, Q297R, P316C, R356N, S361C, G376N, and T393F;
d) T42C, V48C, T72C, P83C, G89C, Y93C, E139N, G162C, N179S, N187A, D194C, P198C, N217C, L222W, T237C, D242C, G247C, S283G, Q297R, P316C, A325G, I341M, R356N, S361C, G376N, and T393F;
e) Y39S, T42C, V48C, T72C, P83C, E85G, G89C, K90R, Y93C, L113M, S125N, K144Q, G162C, S166A, L167F, L169F, D173E, N175S, A178K, Y181L, D194C, P198C, S210T, N217C, L222W, T237C, D242C, G247C, S253Q, S283G, T292H, P316C, A325G, K329S, I341M, N345D, R356N, A357N, S361C, A372K, G376D, and T393S;
f) T42C, V48C, T72C, P83C, G89C, K90R, Y93C, L113M, S125N, K144Q, G162C, S166A, L167F, L169F, D173E, N175S, A178K, D194C, P198C, S210T, N217C, L222W, T237C, D242C, S243A, G247C, S283G, T292H, P316C, A325G, K329S, I341M, N345D, R356N, A357N, S361C, A372Q, G376D, and T393S;
g) T42C, V48C, T72C, P83C, G89C, Y93C, E139N, G162C, N187A, D194C, P198C, N217C, T237C, D242C, G247C, S283G, Q297R, P316C, R356N, S361C, G376N, and T393F;
h) T42C, V48C, T72C, P83C, E85G, G89C, Y93C, L113M, E139N, G162C, S166A, L167F, L169F, N175S, N179S, Y181L, N187A, D194C, P198C, N217C, T237C, D242C, G247C, S283G, T292H, Q297R, P316C, A325G, K329S, I341M, N345D, R356N, A357N, S361C, A372K, G376N, V380T, Y381F, and T393S;
i) T42C, V48C, T72C, P83C, G89C, K90R, Y93C, S125N, K144Q, G162C, L169F, D173E, N175S, A178K, D194C, P198C, S210K, N217C, L222W, T237C, D242C, S243A, G247C, S283G, T292H, P316C, A325G, K329S, I341M, R356N, A357N, S361C, N366H, A372Q, G376D, and T393S;
j) T42C, V48C, T72C, P83C, E85G, G89C, K90R, Y93C, L113M, S125N, K144Q, G162C, S166A, L167F, L169F, D173E, N175S, A178K, Y181L, D194C, P198C, S210T, N217C, L222W, A230S, T237C, D242C, G247C, S253Q, S283G, T292H, K294N, P316C, A325G, K329S, I341M, N345D, R356N, A357N, S361C, A372K, G376D, and T393S;
k) T42C, V48C, T72C, P83C, G89C, Y93C, E139N, G162C, S166A, D173E, N175S, N179S, N187A, D194C, P198C, N217C, T237C, D242C, G247C, S283G, P316C, A325G, K329S, I341L, N345D, R356N, A357N, S361C, G376N, V380T, and T393S;
l) T42C, V48C, T72C, P83C, G89C, Y93C, E139N, G162C, N179S, N187A, D194C, P198C, N217C, T237C, D242C, G247C, S283G, Q297R, P316C, A325G, I341M, R356N, S361C, G376N, and T393F;
m) T42C, V48C, T72C, P83C, G89C, Y93C, E139N, G162C, N187A, D194C, P198C, N217C, T237C, D242C, G247C, S283G, P316C, R356N, S361C, G376N, and T393F;
n) T42C, V48C, T72C, P83C, G89C, K90R, Y93C, S125N, K144Q, G162C, L169F, D173E, N175S, A178K, D194C, P198C, S210K, N217C, L222W, T237C, D242C, G247C, S253K, S283G, T292H, R296E, P316C, A325G, K329S, I341M, R356N, S361C, N366H, A372Q, G376D, and T393S;
o) T42C, V48C, T72C, P83C, G89C, Y93C, G162C, D194C, P198C, N217C, T237C, D242C, G247C, P316C, and S361C;
p) T42C, V48C, T72C, P83C, G89C, K90R, Y93C, S125N, K144Q, G162C, L169F, D173E, N175S, A178K, D194C, P198C, S210K, N217C, L222W, T237C, D242C, S243A, G247C, S253K, S283G, T292H, K294N, P316C, A325G, K329S, I341M, R356N, A357N, S361C, A372Q, G376D, and T393S;
q) T42C, V48C, T72C, P83C, G89C, Y93C, E139N, G162C, S166A, N175S, N179S, N187A, D194C, P198C, N217C, T237C, D242C, G247C, S283G, Q297R, P316C, A325G, K329S, I341L, N345D, R356N, A357N, S361C, G376N, V380T, and T393S;
r) T42C, V48C, T72C, P83C, G89C, Y93C, E139N, G162C, S166A, N179S, N187A, D194C, P198C, N217C, T237C, D242C, G247C, S283G, Q297R, P316C, A325G, I341M, R356N, A357N, S361C, G376N, and T393S;
s) T42C, V48C, T72C, P83C, G89C, Y93C, E139N, G162C, D173E, N187A, D194C, P198C, N217C, T237C, D242C, G247C, S283G, P316C, R356N, S361C, G376N, and T393F;
t) T42C, V48C, T72C, P83C, G89C, K90R, Y93C, S125N, K144Q, G162C, L169F, D173E, A178K, D194C, P198C, N217C, L222W, T237C, D242C, G247C, S283G, T292H, P316C, K329S, I341M, R356N, S361C, A372Q, G376D, and T393S;
u) T42C, V48C, T72C, P83C, G89C, K90R, Y93C, L113M, S125N, K144Q, G162C, S166A, L167F, L169F, D173E, N175S, A178K, D194C, P198C, S210T, N217C, L222W, T237C, D242C, G247C, S283G, T292H, P316C, A325G, K329S, I341M, N345D, R356N, A357N, S361C, A372Q, G376D, and T393S;
v) T42C, V48C, T72C, P83C, G89C, K90R, Y93C, S125N, K144Q, G162C, L169F, D173E, A178K, D194C, P198C, S210K, N217C, L222W, T237C, D242C, G247C, S283G, T292H, K294N, P316C, K329S, I341M, R356N, S361C, A372Q, G376D, and T393S;
w) T42C, V48C, T72C, P83C, G89C, Y93C, E139N, G162C, S166A, N179S, N187A, D194C, P198C, N217C, T237C, D242C, G247C, S283G, Q297R, P316C, A325G, R356N, A357N, S361C, G376N, and T393S;
x) T42C, V48C, T72C, P83C, G89C, Y93C, E139N, G162C, S166A, D173E, N175S, N179S, N187A, D194C, P198C, N217C, T237C, D242C, G247C, S283G, Q297R, P316C, A325G, R356N, A357N, S361C, G376N, V380T, and T393S;
y) T42C, V48C, T72C, P83C, E85G, G89C, Y93C, L113M, E139N, G162C, S166A, L167F, L169F, D173E, N175S, N179S, Y181L, N187A, D194C, P198C, N217C, T237C, D242C, G247C, S283G, T292H, P316C, A325G, K329S, I341M, N345D, R356N, A357N, S361C, A372K, G376N, V380T, Y381F, and T393S;
z) T42C, V48C, T72C, P83C, G89C, K90R, Y93C, S125N, K144Q, G162C, L169F, D173E, A178K, D194C, P198C, S210K, N217C, L222W, T237C, D242C, G247C, S253K, S283G, T292H, R296E, P316C, K329S, I341M, R356N, S361C, N366H, A372Q, G376D, and T393S;
aa) T42C, V48C, T72C, P83C, G89C, K90R, Y93C, S125N, K144Q, G162C, L169F, D173E, A178K, D194C, P198C, S210K, N217C, L222W, T237C, D242C, S243A, G247C, S253K, S283G, T292H, P316C, K329S, I341M, R356N, S361C, N366H, A372Q, G376D, and T393S; or
bb) T42C, V48C, T72C, P83C, G89C, K90R, Y93C, S125N, K144Q, G162C, S166A, L169F, D173E, N175S, A178K, D194C, P198C, S210K, N217C, L222W, T237C, D242C, G247C, S283G, T292H, P316C, A325G, K329S, I341M, N345D, R356N, A357N, S361C, A372Q, G376D, and T393S.

In an embodiment, the present invention provides a nucleic acid sequence encoding the above-described cellulase variant of any one of SEQ ID NO: 5 or SEQ ID NO: 18.

In another embodiment of the present invention, an expression vector comprises the nucleic acid sequence encoding the cellulase variant of any one of SEQ ID NO: 5 or SEQ ID NO: 18.

In an embodiment, the present invention provides a host cell transformed with the expression vector comprising the nucleic acid sequence encoding the cellulase variant of any one of SEQ ID NO: 5 or SEQ ID NO: 18.

In a further embodiment, the present invention provides a method for producing a cellulase variant, said method comprising expressing the cellulase variant in the transformed host cell, wherein the host cell is transformed with the expression vector comprising the nucleic acid sequence encoding the beta-amylase variant of any one of SEQ ID NO: 5 or SEQ ID NO: 18.

In an embodiment, the present invention provides a method of hydrolyzing cellulose of coffee beans, said method comprising drying the coffee beans under exposure to the cellulase variant of any one of SEQ ID NO: 5 or SEQ ID NO: 18.

In another embodiment, the present invention provides a method for aiding or supporting digestion of cellulose in a subject, said method comprising administering to the subject a pharmaceutical composition comprising the cellulase variant of any one of SEQ ID NO: 5 or SEQ ID NO: 18, wherein the cellulose is a component of green vegetables, sprout and/or herbs.

In a further embodiment, the present invention provides a method for refining wood pulp for paper production, said method comprising adding to the wood pulp the cellulase variant of any one of SEQ ID NO: 5 or SEQ ID NO: 18.

In yet another embodiment, the present invention provides a method for deinking paper waste, said method comprising pulping paper waste and adding to the paper-waste pulp the cellulase variant of any one of SEQ ID NO: 5 or SEQ ID NO: 18.

In an embodiment, the present invention provides a method of finishing cellulose-based textile, said method comprising washing the textile with the cellulase variant of any one of SEQ ID NO: 5 or SEQ ID NO: 18.

In an embodiment of the method of finishing cellulose-based textile, the washing occurs during a finishing stage of textile processing and the washing produces an abraded finish.

In another embodiment of the method of finishing cellulose-based textile, the washing occurs during a wet processing stage of textile processing and the washing produces a biopolished finish.

In an embodiment, the present invention provides a detergent or stain-removing spotting concentrate comprising the cellulase variant of any one of SEQ ID NO: 5 or SEQ ID NO: 18.

In a further embodiment, the present invention provides a method of laundering a cellulose-based textile, said method comprising washing the cellulose-based textile with the detergent or stain-removing spotting concentrate comprising the cellulase variant of any one of SEQ ID NO: 5 or SEQ ID NO: 18.

In an embodiment of the method of laundering a cellulose-based textile, the washing improves color brightness, feel of the cellulose-based textile and dirt removal compared to laundered cellulose-based textile which does not comprise the cellulase variant.

### Cyclodextrin Glucanotransferase Variants

Cyclodextrin glucanotransferase is an enzyme used in industry to produce cyclodextrins, a family of cyclic α-(1, 4)-linked oligosaccharides, from amyloase, starches, such as corn and potatoes, and other polysaccharides. Cyclodextrin glucanotransferase, known as cyclodextrin glucosyltransferase, cyclodextrin glucanotransferase, or simply CGTase, also is used as a catalyst in synthesis of glycosylated molecules and as anti-staling agents in the baking industry. Cyclodextrin glucanotransferase is found in many bacteria, including Bacillus megaterium, Bacillus circulans, Bacillus macerans, Bacillus ohbensis and Bacillus stearothermophilus, as well in as some archaea.

The cyclodextrin glucanotransferase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type cyclodextrin glucanotransferase will denature, decompose and/or under which the cyclodextrin glucanotransferase enzymatic function is significantly reduced or eliminated.

The present invention provides a cyclodextrin glucanotransferase variant of a wild-type cyclodextrin glucanotransferase from Bacillus macerans of SEQ ID NO: 6 comprising mutations:
a) D34S, D64G, R70C, I84V, D141S, L152A, L154A, I155K, D160I, R171A, G177S, K239E, Q260H, Y261L, D277S, E303R, E316A, D323K, K329S, L335R, A336Q, E339Q, I345A, D358T, S365N, D397G, G412T, A421K, T436G, P471S, N531H, I542T, K578Q, A585T, T589Y, K594T, T599G, K605E, S606P, A638V, T643N, P646T, S663T, V706T, and D712T;
b) D34S, K36T, D64G, R70C, I84V, D86N, K115N, D141S, D150N, L152A, L154A, I155K, T156N, S157L, D160V, R161M, V168T, R171A, G177S, E180D, L191M, F238L, K239E, F255H, Q260H, Y261L, G275S, D277S, E303R, E316A, R322A, D323K, T325N, K329W, V334M, L335R, A336Q, S337K, E339A, Q341D, D343N, Y344W, I345A, M348L, D358S, V362Y, G364N, S365G, G366S, A369K, T370Y, E371H, D397G, G412T, V418I, A421K, T436G, R440V, N443T, S467T, A468T, A469S, P471S, S473T, S477T, S526T, N531H, I542T, A554K, G568N, K578Q, A585T, A586T, T589A, K594T, T599G, A600T, F604Y, K605V, S606P, T630E, A638V, A639S, T643N, P646T, Y654F, I658M, S663N, I681L, T690V, V706T, and D712T;
c) D34S, D64G, R70C, I84V, D141S, L152A, L154A, I155K, D160I, R171A, G177S, F238L, K239E, Q260H, Y261L, D277S, E303R, E316A, D323K, K329S, L335R, A336Q, E339Q, D343N, I345A, D358T, S365N, D397G, G412T, A421K, T436G, P471S, N531H, I542T, K578Q, A585T, T589Y, K594T, T599G, K605E, S606P, A638V, T643N, P646T, S663T, V706T, and D712T;
d) D34S, D64G, R70C, I84V, D141S, L152A, D160I, R161M, G177S, F238L, Q260H, Y261L, D277S, E303R, E316K, D323K, K329F, L335R, A336L, D397G, A421K, T436G, T438W, N443T, P471S, I542T, A585T, K594T, T599G, K605E, T643N, P646T, V706T, and D712T;
e) D34S, D64G, R70C, I84V, D141S, L152A, D160I, R161M, G177S, F238L, Q260H, Y261L, D277S, E303R, E316K, D323K, K329F, L335R, A336Q, D397G, A421K, T436G, T438W, N443T, P471S, I542T, K578T, A585T, K594Q, T599G, N602S, K605E, T643N, P646T, V706T, and D712T;
f) D34S, D64G, R70C, I84V, D141S, L152A, I155K, D160I, R161M, R171A, G177S, F238L, Q260H, Y261L, D277S, E303R, E316A, D323K, K329S, L335R, A336Q, E339Q, I345A, S365N, D397G, A421K, T436G, N443T, P471S, N531H, I542T, A585T, K594T, T599G, K605E, A638V, T643N, P646T, V706T, and D712T;
g) D34N, K36T, D56N, D64G, R70C, I84V, D86N, K115N, Q134K, D141S, D150N, L152A, L154A, I155K, T156N, S157L, D160I, R161M, V168T, R171A, N175D, G177S, E180D, D186N, S189N, L191M, L201Y, F238L, K239E, L244W, V250F, F255H, Q260H, Y261L, G264P, S268T, G275S, D277S, Y287F, E303R, E316A, R322A, D323K, T325N, K329W, V334M, L335R, A336L, S337K, E339A, Q341D, D343N, Y344W, I345A, M348L, D358S, V362Y, G364N, S365G, G366S, A369K, T370Y, E371H, D397G, G412T, V418I, A421K, S428L, T436G, T438W, E439Q, R440V, N443T, S455G, S467T, A468T, A469T, P471S, S473T, S477T, N507T, S526T, N531H, I542T, A554K, G568N, K578Q, I581V, A585T, A586T, T589A, K594T, T599G, A600T, N602S, F604Y, K605V, S606P, L620V, T630Q, A638V, A639S, T643N, P646T, Y654F, S663T, K675Q, I681V, K684R, T690V, A703T, V706T, and D712T;
h) D34S, D64G, R70C, I84V, D141S, L152A, I155K, D160I, R161M, R171A, G177S, F238L, Q260H, Y261L, D277S, E303R, E316A, D323K, K329S, L335R, A336L, E339Q, I345A, S365N, D397G, A421K, T436G, T438W, P471S, N531H, I542T, A585T, K594T, T599G, K605V, A638V, T643N, P646T, V706T, and D712T;
i) D34S, D64G, R70C, I84V, K115N, D141S, L152A, L154A, I155K, D160I, R171A, G177S, E180D, L191M, F238L, K239E, Q260H, Y261L, D277S, E303R, E316A, F321I, D323K, K329S, V334M, L335R, E339Q, I345A, D358T, V362Y, S365G, G366S, T370Y, E371H, D397G, G412T, A421K, T436G, A469T, P471S, S477T, N531H, I542T, K578Q, A585T, T589Y, K594T, T599G, F604Y, K605V, S606P, T630E, A638T, T643N, P646T, S663T, V706T, and D712T;
j) D34S, K36T, D64G, R70C, I84V, D86N, K115N, D141S, D150N, L152A, L154A, I155K, T156N, S157L, D160I, R161M, V168T, R171A, G177S, E180D, L191M, K239E, L244W, F255H, Q260H, Y261L, G275S, D277S, E303R, Y313L, E316A, R322A, D323K, T325N, K329S, V334M, L335I, A336Q, S337R, E339A, Q341D, D343N, Y344W, I345A, M348L, D358S, V362Y, G364N, S365G, G366S, A369K, T370Y, E371H, D397G, G412T, V418I, A421K, T436G, S467T, A468T, A469T, P471S, S473T, S477T, S526T, N531H, I542T, A554K, G568N, K578Q, A585T, A586T, T589A, K594T, T599G, A600Q, F604Y, K605V, S606P, T630E, A638V, A639S, T643N, P646T, Y654F, S663N, I681L, T690V, V706T, and D712T;
k) D34S, K36T, D64G, R70C, I84V, D86N, K115N, D141S, D150N, L152A, L154A, I155K, T156N, S157L, D160I, R161M, V168T, R171A, G177S, E180D, L191M, K239E, F255H, Q260H, Y261L, G275S, D277S, E303R, E316A, R322A, D323K, T325N, K329W, V334M, L335R, A336Q, S337R, E339A, D343N, Y344W, I345A, D358S, V362Y, G364N, S365G, G366S, T370Y, E371H, D397G, G412T, V418I, A421K, T436G, R440V, A468T, A469T, P471S, S473T, S477T, N531H, I542T, A554K, G568N, K578T, A585T, T589A, K594Q, T599G, A600T, N602S, F604Y, K605V, S606P, T630E, A638V, A639S, T643N, P646T, Y654F, S663N, I681L, V706T, and D712T;
l) D34S, D64G, R70C, I84V, K115N, D141S, L152A, L154A, I155K, D160I, R171A, G177S, E180D, L191M, K239E, Q260H, Y261L, D277S, E303R, E316A, F321I, D323K, T325H, K329S, V334M, L335R, A336Q, D343N, I345A, D358T, V362Y, S365G, G366S, T370Y, E371H, D397G, G412T, A421K, T436G, A469T, P471S, S477T, N531H, I542T, K578Q, A585T, T589Y, K594T, T599G, F604Y, K605V, S606P, T630E, A638T, T643N, P646T, S663T, V706T, and D712T;
m) D34S, K36T, D64G, R70C, I84V, D86N, K115N, D141S, D150N, L152A, L154A, I155K, T156N, S157L, D160I, V168T, R171A, G177S, E180D, L191M, F238L, K239E, F255H, Q260H, Y261L, G275S, D277S, E303R, E316A, R322A, D323K, T325N, K329W, V334M, L335R, A336Q, S337R, E339Q, D343N, Y344W, I345A, D358S, V362Y, G364N, S365G, G366S, T370Y, E371H, D397G, G412T, V418I, A421K, T436G, R440V, A468T, A469T, P471S, S473T, S477T, N531H, I542T, A554K, G568N, K578Q, A585T, T589A, K594T, T599G, A600T, F604Y, K605V, S606P, T630E, A638V, A639S, T643N, P646T, Y654F, S663N, V706T, and D712T; or
n) D34N, K36T, D56N, D64G, R70C, I84V, D86N, K115N, Q134K, D141S, D150N, L152A, L154A, I155K, T156N, S157L, D160I, R161M, V168T, R171A, N175D, G177S, E180D, D186N, S189N, L191M, L201Y, K239E, V250F, F255H, Q260H, Y261L, G264P, S268T, G275S, D277S, Y287F, E303R, E316A, R322A, D323K, T325N, K329S, V334M, L335R, A336L, S337R, E339Q, Q341D, Y344W, I345A, M348L, D358S, V362Y, G364N, S365G, G366S, A369K, T370Y, E371H, D397G, G412T, V418I, A421K, S428L, T436G, T438W, E439Q, V442M, S455G, S467T, A468T, A469T, P471S, S473T, S477T, N507T, S526T, N531H, I542T, A554K, G568N, K578Q, I581V, A585T, A586T, T589A, K594T, T599G, A600T, F604Y, K605V, S606P, L620V, T630Q, A638V, A639S, T643N, P646T, Y654F, S663T, K675Q, I681V, K684R, T690V, A703T, V706T, and D712T.

The present invention also provides a cyclodextrin glucanotransferase variant of a wild-type cyclodextrin glucanotransferase from Geobacillus stearothermophilus of SEQ ID NO: 7 comprising mutations:
a) I44V, T72C, G79C, I161M, D186N, N212T, F222Y, Q232N, F233T, K239L, I242A, M255H, F263H, M270Q, D271H, D275N, P278N, H297Y, E302N, N322D, D326N, N331H, A340D, E343W, M360H, A361S, D362L, E363N, P366N, K368R, V369L, V375F, V382T, N396G, M404D, R413T, Q419R, S423A, D435S, S470T, T484K, Q486V, A489G, G499N, A503N, G510Q, G515S, A522T, V525H, M534T, K541Q, V553K, V562Q, Q590V, Q595G, V596E, A598N, W624S, L649M, V662Y, G669D, K670T, K678I, K679R, G681D, I687T, S701N, T705K, V706I, N707I, and N709D;
b) T72C, G79C, I161A, D186N, N212T, F233T, F263Q, M270Q, D271H, D275N, P278N, H297Y, E302N, D326T, A340D, E343W, M360L, L373M, V375F, V382T, M404D, R413T, D435S, S470T, T484E, A503N, G510Q, A522T, V525H, K541Q, Q590V, Q595G, A598N, L649M, G681D, I687T, S701N, T705K, V706I, N707I, and N709D;
c) T72C, G79C, I161A, D186N, N212T, F233T, I242A, F263Q, S268E, M270Q, D271N, D275N, P278G, H297Y, E302N, D326T, A340D, E343W, M360L, P366N, K368R, V369L, V375F, V382T, M404D, R413T, Q419R, S423A, D435S, E449K, S470T, A489G, G499N, A503N, G510Q, G515S, A522T, V525H, K541Q, V553K, V562Q, Q590V, Q595G, A598N, L649M, K678I, G681D, I687T, S701N, T705K, V706I, N707I, and N709D;
d) T72C, G79C, I161A, D186N, F233T, F263Q, M270Q, D271N, D275N, P278N, H297Y, E302N, D326T, A340D, E343W, M360L, V375F, V382T, M404D, R413T, D435S, S470T, T484E, A503N, G510Q, A522T, V525H, K541Q, Q590V, A598N, L649M, G681D, I687T, S701N, T705K, V706I, N707I, and N709D;
e) T72C, G79C, I161A, D186N, N212T, F222Y, Q232N, F233T, K239L, I242A, F263H, S268E, M270Q, D271N, D275N, P278N, H297Y, E302N, D326N, A340D, E343W, M360H, E363N, P366N, K368R, V369L, V375F, V382T, M404D, R413T, Q419R, S423A, D435S, S470T, Q486V, A489G, G499N, A503N, G510Q, G515S, A522T, V525H, M534T, K541Q, V553K, V562Q, Q590V, Q595G, V596E, A598N, L649M, V662Y, K678I, K679R, G681D, I687T, S701N, T705K, V706I, N707I, and N709D;
f) I44V, T72C, G79C, I161M, D186N, S200G, N212T, F222Y, Q232N, F233T, K239I, I242A, M255H, F263Q, S268K, M270Q, D271N, D275N, P278N, H297Y, E302N, L316V, V319A, N322D, D326T, N331H, A340D, D342N, E343W, M360L, A361S, D362L, E363N, P366N, K368R, V369L, V375F, V382T, N396G, M404D, R413T, Q419R, S423A, D435S, T436Q, L465S, S470T, T484E, A489G, G499N, A503N, G510Q, G515S, A522T, V525H, M534S, K541Q, V553K, V562Q, Q590V, Q595G, V596E, A598N, W624S, S643D, L649M, V662Y, E668A, K670T, K678I, G681D, I687T, S701N, T705K, N707I, and N709D;
g) I44V, S65A, E96D, A113S, D119S, T122A, T135P, I191L, T198A, S200M, N212S, F227L, Q232P, F233V, K236R, I242V, L244M, L246I, F269L, V273I, Y274D, D275N, S294A, F298Y, D326N, I345L, A361I, E363G, I371M, T406S, S427N, S432A, S443G, I447V, L465S, K467S, S468N, S480A, A489G, A503S, N508D, T518A, A522T, V525T, I531V, K541Q, L542V, V553T, N560T, V562A, S564N, T574V, T576A, I580V, P581S, A582P, T591S, G593S, V596T, N621T, W624L, E626Q, L630I, H635Y, N641D, L649M, I654V, V662I, G681S, S682Q, I686T, S700N, T704K, V705I, N706I, and N708D;
h) I44V, T72C, G79C, I161M, D186N, S200G, N212T, F222Y, Q232N, F233T, K239I, I242A, L244W, M255H, F263H, D271H, D275N, P278N, S288G, E289G, N295D, H297Y, E302N, K315A, Q318E, V319S, N322D, S324N, D326T, N331H, T337Q, A338Q, A340D, D342N, E343W, M360L, A361S, D362L, E363N, P366N, K368R, V369L, V375F, V382T, N396G, M404D, R413T, Q419R, S423A, D435S, T436Q, I447V, L465S, S470T, T484K, A489G, G499N, N501D, A503N, G510Q, G515A, A522T, E523T, V525H, M534S, H540N, K541Q, T551D, V553K, V562Q, Q572E, T576S, Q590V, T591V, Q595G, V596K, A598N, A599N, D601V, W624S, S643D, L649M, V662Y, E668A, K670T, K675M, K678I, K679R, G681D, I687T, S690N, S701N, T705K, N707I, and N709D;
i) T72C, G79C, I161A, D186N, N212T, F233T, F263Q, S268E, D271N, D275N, P278N, H297Y, E302N, D326T, N331Y, A340D, E343W, M360L, P366N, K368R, V369L, V375F, V382T, M404D, R413T, Q419R, S423A, D435S, E449K, S470T, T484E, A489G, G499N, A503N, G510Q, G515S, A522T, V525H, K541Q, V553K, V562Q, Q590V, Q595G, A598N, L649M, K678I, G681D, I687T, S701N, T705K, V706I, N707I, and N709D;
j) T72C, G79C, I161C, D186N, N212T, F222Y, Q232N, F233T, K239L, I242A, F263H, S268E, M270Q, D271N, D275N, P278N, H297Y, E302N, N322D, D326N, A340D, E343W, M360L, E363N, P366N, K368R, V369L, V375F, V382T, M404D, R413T, Q419R, S423A, D435S, S470T, T484K, A489G, G499N, A503N, G510Q, G515S, A522T, V525H, M534T, K541Q, V553K, V562Q, Q590V, Q595G, V596E, A598N, L649M, V662Y, K678I, K679R, G681D, I687T, S701N, T705K, V706I, N707I, and N709D;
k) T72C, G79C, I161C, D186N, F233T, I242A, F263Q, M270Q, D271N, D275N, P278N, H297Y, E302N, D326T, A340D, E343W, M360L, L373M, V375F, V382T, M404D, R413T, D435S, S470T, T484E, A503N, G510Q, A522T, V525H, K541Q, Q590V, A598N, L649M, G681D, I687T, S701N, T705K, V706I, N707I, and N709D;
l) T72C, G79C, I161A, D186N, N212T, F233T, I242A, F263Q, M270Q, D271H, D275N, P278N, H297Y, E302N, D326T, A340D, E343W, M360L, L373M, V375F, V382T, M404D, R413T, D435S, S470T, T484E, A503N, G510Q, A522T, V525H, K541Q, Q590V, Q595G, A598N, L649M, G681D, I687T, S701N, T705K, V706I, N707I, and N709D;
m) I44V, T72C, G79C, I161M, D186N, S200G, N212T, F222Y, Q232N, F233T, K239I, I242A, M255H, F263Q, S268K, D271N, D275N, P278N, H297Y, E302N, L316I, V319A, D326T, N331H, A340D, D342N, E343W, M360H, A361S, D362L, E363N, P366N, K368R, V369L, V375F, N396G, M404D, R413T, Q419R, S423A, D435S, T436Q, L465S, S470T, T484E, A489G, G499N, A503N, G510Q, G515S, A522T, V525H, M534S, K541Q, V553K, V562Q, Q590V, Q595G, V596K, A598N, W624S, S643D, L649M, V662Y, E668A, G669D, K670T, K678I, K679R, G681D, I687T, S701N, T705K, N707I, and N709D;
n) I44V, T72C, G79C, I161M, D186N, N212T, F222Y, Q232N, F233T, K239L, I242A, M255H, K259R, F263H, S268K, M270Q, D271H, D275N, P278G, H297Y, E302N, L316V, D326N, N331H, A340D, E343W, M360H, A361S, D362L, E363N, P366N, K368N, V369L, V375F, V382T, N396G, M404D, R413T, Q419R, S423A, D435S, S470T, T484E, A489G, G499N, A503N, G510Q, G515S, A522T, V525H, M534T, K541Q, V553K, V562Q, Q590V, Q595G, V596E, A598N, W624S, L649M, V662Y, G669D, K670T, K678I, K679R, G681D, I687T, S701N, T705K, V706I, N707I, and N709D;
o) T72C, G79C, G681D, I687T, S701N, T705K, V706I, N707I, and N709D; or
p) I44V, T72C, G79C, E96D, I161M, D186N, S200G, N212T, F222Y, Q232N, F233T, K239L, I242A, L244W, M255H, F263H, S268K, M270Q, D271N, D275N, P278N, S288G, E289G, N295D, H297Y, E302N, K315A, Q318E, V319A, N322D, S324N, D326T, N331H, T337Q, A340D, D342N, E343W, M360L, A361S, D362L, E363N, P366N, K368R, V369L, V375F, V382T, N396G, M404D, N411T, R413T, Q419R, S423A, D435S, T436Q, R439L, I447V, L465S, S470T, T484E, A489G, G499N, N501D, A503N, G510Q, G515A, E523T, V525H, M534S, H540N, K541Q, T551D, V553K, V562Q, Q572E, T576S, Q590V, T591V, Q595G, V596K, A598N, A599N, D601V, W624S, S643D, L649M, V662Y, E668A, G669D, K670T, K675M, K678I, K679R, G681D, I687T, S690N, S701N, T705K, N707I, and N709D.

In an embodiment, the present invention provides a nucleic acid sequence encoding the above-described cyclodextrin glucanotransferase variant of any one of SEQ ID NO: 6 or SEQ ID NO: 7.

In another embodiment of the present invention, an expression vector comprises the nucleic acid sequence encoding the cyclodextrin glucanotransferase variant of any one of SEQ ID NO: 6 or SEQ ID NO: 7.

In an embodiment, the present invention provides a host cell transformed with the expression vector comprising the nucleic acid sequence encoding the cyclodextrin glucanotransferase variant of any one of SEQ ID NO: 6 or SEQ ID NO: 7.

In a further embodiment, the present invention provides a method for producing a cyclodextrin glucanotransferase variant, said method comprising expressing the cyclodextrin glucanotransferase variant in the transformed host cell, wherein the host cell is transformed with the expression vector comprising the nucleic acid sequence encoding the cyclodextrin glucanotransferase variant of any one of SEQ ID NO: 6 or SEQ ID NO: 7.

In an embodiment, the present invention provides a catalyst comprising the cyclodextrin glucanotransferase variant of any one of SEQ ID NO: 6 or SEQ ID NO: 7.

In another embodiment, the present invention provides a method for improving anti-staling properties of a baked food product, said method comprising treating the baked food product with the cyclodextrin glucanotransferase variant of any one of SEQ ID NO: 6 or SEQ ID NO: 7, thereby improving the anti-staling properties of the treated baked food product compared to a baked food product which has not been treated with the cyclodextrin glucanotransferase variant.

### Glutaminase Variants

Glutaminase is an enzyme which catalyzes the hydrolysis of glutamine at the amide bond, to form glutamate (glutamic acid) and ammonia. Glutaminase is produced by bacteria, yeast, filamentous fungi and molds. High salt-tolerant glutaminases are used in industrial applications, such as food fermentation that require a high salt environment. Glutaminase, also known as L-glutaminase, is an aroma- and flavor-enhancer in the food industry, including in fermented food products, such as soy sauce and fermented condiments. Glutaminase has replaced the use of the food allergen monosodium glutamate, e.g., in Chinese food.

Glutaminase of microbial origin has been shown to exhibit antitumor, anti-leukemic and anti-HIV activity; therefore, glutaminases may be administered as therapeutic enzymes. Studies have shown that cancer cells consume about 15 times the amount of L-glutamine for their growth than is consumed by other cells under hypoxic conditions; however, cancer cells do not produce glutamine, whereas normal cells do. Thus, reducing glutamine levels in tumors and cancers, such as melanoma and leukemia, and inhibiting tumor and cancer cell growth by L-glutaminase are therapeutic applications of this enzyme and of the glutaminase variants provided herein. The glutaminase variants may be formulated as an active ingredient in pharmaceutical compositions for treatment of cancers and HIV-infected cells.

The glutaminase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type glutaminase will denature, decompose and/or under which the glutaminase enzymatic function is significantly reduced or eliminated.

The present invention provides a glutaminase variant of a wild-type glutaminase from Bacillus amyloliquefaciens of SEQ ID NO: 8 comprising mutations:
a) L14M, A15Q, V22I, H24E, P27K, F28Y, S29T, V30S, N31Q, S34C, N36D, G42A, E43K, I44V, D46K, S47N, I53M, L54C, E55T, P56T, M60C, S80T, A83L, S87D, R88N, V99M, R111N, I114M, K116N, G118A, L135M, H142E, N143E, E146G, Y147R, V151F, E153R, I156A, K158R, P160L, H161T, H163N, F167Y, T173H, E186S, F189N, E199D, L202F, K203Q, S209V, T211C, L216R, I217M, L219A, I220Y, H223N, R231G, K241Q, K244L, L248M, V265L, S282H, E286Q, Q287A, H317Q, I318M, and E323G;
b) A15Q, P27K, S34C, G42A, D46K, S47N, E55T, P56T, A83L, R111N, K116N, N143E, Y147R, V151F, H163N, F189I, S209V, T211C, L216R, L219A, H223N, R231Q, K241Q, K244L, L248M, S282H, E286Q, Q287A, and I318M;
c) I318M;
d) H24G;
e) Q19H, H24Y, S29A, V30A, K33Q, I53V, S59T, E65D, P69S, L72M, H115N, I130L, V132I, L135I, T140S, H142Y, N143E, Y147F, F149Y, F152M, K154T, H161R, Q172E, D193E, R231H, H290Q, N291S, and I318M;
f) A15Q, H24E, P27K, F28Y, S29A, N31Q, S34C, G42A, E43K, D46K, S47N, L54C, E55T, P56T, M60C, H62G, S80T, A83L, R111N, K116N, F121C, L135M, P137R, N143E, E146G, Y147R, V151F, P160L, H161T, H163N, E186S, F189M, L202F, K203Q, S209V, T211C, L216R, I217M, L219A, H223N, R231G, K241Q, K244L, L248M, V265L, S282H, E286Q, Q287A, I318M, and E323D;
g) A15Q, P27K, S34C, G42A, D46K, S47N, E55T, P56T, H62G, A83L, R111N, K116N, P137R, N143E, Y147R, V151F, H163N, F189M, S209V, T211C, L216R, L219A, H223N, R231G, K241Q, K244L, L248M, S282H, E286Q, Q287A, and I318M;
h) P27K, S34C, D46K, S47N, E55Y, H62G, A83L, R111N, K116N, P137R, N143E, Y147R, V151F, H163N, F189M, S209V, T211C, L216K, L219A, R231Q, K241Q, L248M, S282H, E286Q, Q287A, and I318M;
i) A15Q, H24E, P27K, F28Y, S29A, N31Q, S34C, G42A, E43K, D46K, S47N, L54C, E55T, P56T, M60C, H62G, S80T, A83L, R111N, K116N, L135M, P137R, N143E, E146N, Y147R, V151F, P160L, H161T, H163N, E186S, F189M, L202F, K203Q, S209V, T211C, L216R, I217M, L219A, H223N, R231G, K241Q, K244L, L248M, V265L, S282H, E286Q, Q287A, I318M, and E323D; and
j) L14M, A15Q, H24E, P27K, F28Y, S29A, V30S, N31Q, S34C, N36D, G42A, E43K, D46K, S47N, I53M, L54C, E55T, P56T, M60C, S80T, A83L, R88N, V99M, R111N, I114M, K116N, G118A, L135M, H142E, N143E, E146G, Y147R, V151F, K158R, P160L, H161T, H163N, F167Y, T173H, E186S, F189I, L202F, K203Q, S209V, T211C, L216R, I217M, L219A, I220Y, H223N, R231G, K241Q, K244L, L248M, V265L, S282H, E286A, Q287A, H317Q, I318M, and E323D;
k) A15Q, P27K, F28Y, S29T, S34C, G42A, D46K, S47N, E55T, P56K, M60C, H62G, A83L, R111N, K116N, P137Q, N143E, Y147R, V151F, H161T, H163N, F189M, L202F, S209V, T211C, L216R, I217M, L219A, H223N, R231Q, K241Q, K244L, L248M, V265L, S282H, E286Q, Q287A, I318M, and E323D;
l) A15Q, P27K, F28Y, S29T, S34C, G42A, D46K, S47N, E55T, P56K, M60C, A83L, R111N, K116N, P137Q, N143E, Y147R, V151F, P160L, H161T, H163N, F189I, L202F, S209V, T211C, L216R, I217M, L219A, H223N, R231G, K241Q, K244L, L248M, V265L, S282H, E286Q, Q287A, I318M, and E323D;
m) A15Q, P27K, S29T, S34C, G42A, D46K, S47N, E55T, P56T, M60C, H62G, A83L, R111N, K116N, P137R, N143E, Y147R, V151F, H163N, F189M, L202F, S209V, T211C, L216R, L219A, H223N, R231G, K241Q, K244L, L248M, S282H, E286Q, Q287S, I318M, and E323D;
n) P27K, S34C, D46K, S47N, E55Y, H62G, A83L, R111N, K116N, N143E, Y147R, V151F, H163N, F189M, S209V, T211C, L216K, L219A, R231Q, K241Q, L248M, S282H, E286Q, Q287A, and I318M;
o) L14M, A15Q, H24E, P27K, F28Y, S29T, V30S, N31Q, S34V, N36D, G42A, E43K, D46K, S47N, I53M, L54C, E55T, P56T, M60C, H62G, S80T, A83L, R88N, V99M, R111N, K116N, L135M, P137R, H142E, N143E, E146G, Y147R, V151F, K158R, P160L, H161T, H163N, F167Y, T173H, E186S, F189M, L202F, K203Q, S209V, T211C, L216R, I217M, L219A, I220Y, H223N, R231G, K241Q, K244L, L248M, V265L, S282H, E286A, Q287S, H317Q, I318M, and E323D;
p) A15Q, P27K, S29A, S34C, G42A, D46K, S47N, E55T, P56T, M60C, H62G, A83L, R111N, K116N, N143E, Y147R, V151F, H163N, F189I, L202F, S209V, T211C, L216R, L219A, H223N, R231G, K241Q, K244L, L248M, S282H, E286Q, Q287A, I318M, and E323D; or
q) L14M, A15Q, V22I, H24E, P27K, F28Y, S29A, V30K, N31E, S34C, N36D, G42A, E43K, I44V, D46K, S47N, I53M, L54C, E55T, P56T, M60C, H62G, S80T, A83L, S87D, R88N, V99M, R111N, I114M, K116N, G118A, L135M, P137Q, H142E, N143E, E146G, Y147R, V151F, E153R, I156A, K158R, P160L, H161T, H163N, F167Y, T173H, E186S, F189N, E199D, L202F, K203Q, S209V, T211C, L216R, I217M, L219A, I220Y, H223N, R231G, K241Q, K244L, L248M, V265L, S282H, E286Q, Q287A, H317Q, I318M, and E323G.

In an embodiment, the present invention provides a nucleic acid encoding the glutaminase variant of SEQ ID NO: 8.

In another embodiment of the present invention, an expression vector comprises the nucleic acid encoding the glutaminase variant of SEQ ID NO: 8.

In an embodiment, the present invention provides a host cell transformed with the expression vector comprising the nucleic acid sequence encoding of SEQ ID NO: 8.

In a further embodiment, the present invention provides a method for producing a glutaminase variant, said method comprising expressing the glutaminase variant in the transformed host cell, wherein the host cell is transformed with the expression vector comprising the nucleic acid sequence encoding the glutaminase variant of SEQ ID NO: 8.

In an embodiment, the present invention provides a method of enhancing the flavor of a fermented food product, said method comprising adding to the food product the glutaminase variant of SEQ ID NO: 8.

In an embodiment of the method of enhancing the flavor of a fermented food product, the fermented food product is a soy sauce or a condiment.

In another embodiment, the present invention provides a method for treating a disease or disorder, said method comprising administering to a subject a pharmaceutical composition comprising the glutaminase variant of SEQ ID NO: 8, wherein the subject has a disease or disorder in which abnormal cells utilize L-glutamine for growth and/or survival.

In another embodiment of the method, the abnormal cells are leukemic cells or cells infected with human immunodeficiency virus (HIV).

### Protease Variants

Proteases, also called proteolytic enzymes or peptidases, are a group of enzymes that hydrolyze peptide bonds, thereby breaking down protein (proteolysis) into smaller polypeptides or amino acids. Each type of proteases, such as pepsin, trypsin, chymotrypsin, papain, bromelain, carboxypeptidase, aminopeptidase, dipeptidase, and bacterial subtilisin, breaks a specific peptide bond.

Proteases are important in digestion to breakdown protein in foods into amino acids that are then absorbed into tissues and used by the body to create new protein, including muscle, antibodies, blood cells and hormones. Of the twenty amino acids in the body's proteins, the following nine are essential, since they cannot be produced by cells: histidine, isoleucine, leucine, lysine, methionine, phenylalanine, threonine, tryptophan and valine.

Protease deficiency causes protein deficiency and an alkaline environment in the blood, since protein digestion, which causes acidity, is lacking. Moreover, since protein carries protein-bound calcium, a protease deficiency can lead to calcium-deficiency diseases, such as arthritis, osteoporosis, gout, sciatica, bone spurs and/or disk degeneration. Protease deficiency can cause hypoglycemia from insufficient protein digestion, leading to inability to produce glucose when needed. Since proteases digest bacteria and viruses, a deficiency in proteases can result in susceptibility to infection by bacteria, viruses and yeast. Deficiency in or abnormal proteolysis causes diseases, such as pancreatitis; inflammatory diseases, such as rheumatoid arthritis; inflammatory bowel syndrome, and possibly Alzheimer's disease and diabetes. In addition to the administration of proteases to aid or support digestion, protease is used in various medical applications, including treatment of ischemic stroke by enabling plasmin breakdown of blood clots; removal of dead/damaged tissue in wounds; as an auxiliary to antibiotic therapy, e.g., to remove biofilm produced by bacterial infections; as a component of pancreatic enzyme replacement therapy; and as an anti-inflammatory agent. Proteases are also used as additives to laundry detergents and in baking as a flour treatment agent/dough conditioner to improve dough plasticity and extensibility.

The protease variants provided herein are stable under conditions such as high temperature, e.g., over 50° C., pH extremes, e.g., high acidity at pH of 4 or lower, in the presence of salts, alkalis and/or surfactants, and other such severe conditions under which wild-type protease will denature, decompose and/or under which the protease enzymatic function is significantly reduced or eliminated.

The present invention provides a protease variant of wild-type protease from Bacillus amyloliquefaciens of SEQ ID NO: 9 comprising mutations:
a) F339S, A347D, E350P, Q353G, M354I, V355A, L357V, F362H, K364R, Q365P, E375G, D377E, F381Q, T382V, G384E, T385K, R405K, D409K, K410R, K411P, I412L, and S415G;
b) F339S, D340E, A347D, E350P, Q353G, M354I, V355A, L357V, F362H, K364R, Q365P, E375G, D377E, F381Q, T382V, G384E, T385K, R405K, D409K, K410R, K411P, I412L, and S415Y;
c) F339S, D340E, A347D, E350P, Q353G, M354I, V355A, L357V, F362H, K364R, Q365P, E375G, D377E, F381Q, T382V, G384E, T385K, R405K, D409K, K410R, K411P, I412L, and S415G;
d) F339S, D340E, A347G, E350P, Q353G, M354I, V355A, L357V, F362H, K364R, Q365P, E375G, D377E, F381Q, T382V, G384E, T385K, R405K, D409K, K410R, K411P, I412L, Y413F, and S415G;
e) F339S, D340E, A347G, E350P, Q353G, M354I, V355A, L357V, F362H, K364R, Q365P, E375G, D377E, F381Q, T382V, G384E, T385K, R405K, D409K, K410R, K411P, I412L, Y413F, and S415Y;
f) F339R, D340E, A347G, E350P, Q353G, M354I, V355A, L357V, F362H, K364R, Q365P, D377E, F381Q, T382V, G384E, T385K, R405K, D409K, K410R, K411P, I412L, Y413F, and S415Y;
g) F339S, A347D, E350P, Q353G, M354I, V355A, L357V, F362H, Q365P, E375G, D377E, F381Q, T382V, G384E, T385K, R405K, D409K, K410R, K411P, I412L, and S415G;
h) F339S, A347D, E350P, Q353G, M354I, V355A, L357V, F362H, K364R, Q365P, E375G, D377E, F381Q, T382V, G384E, T385K, R405K, D409K, K410R, K411P, I412L, Y413K, and S415G;
i) F339S, D340E, A347D, E350P, Q353G, M354I, V355A, L357V, F362H, K364R, Q365P, E375G, D377E, F381Q, T382V, G384E, T385K, R405K, D409K, K410R, K411P, I412L, Y413K, and S415G; or
j) F339S, D340E, A347G, E350P, Q353G, M354I, V355A, L357V, F362H, K364R, Q365P, E375G, D377E, F381Q, T382V, G384E, T385K, R405K, D409K, K410R, K411P, I412L, Y413K, and S415G.

In an embodiment, the present invention provides a nucleic acid sequence encoding the above-described protease variant of SEQ ID NO: 9.

In another embodiment of the present invention, an expression vector comprises the nucleic acid sequence encoding the protease variant of SEQ ID NO: 9.

In an embodiment, the present invention provides a host cell transformed with the expression vector comprising the nucleic acid sequence encoding the protease variant of SEQ ID NO: 9.

In a further embodiment, the present invention provides a method for producing a protease variant, said method comprising expressing the protease variant in the transformed host cell, wherein the host cell is transformed with the expression vector comprising the nucleic acid sequence encoding the protease variant of SEQ ID NO: 9.

The present invention also provides a method for aiding or supporting digestion of protein in a subject, said method comprising administering to the subject a pharmaceutical composition comprising the protease variant of SEQ ID NO: 9.

In an embodiment, the present invention provides a detergent or stain-removing spotting concentrate comprising the protease variant of SEQ ID NO: 9.

In another embodiment, the present invention provides a method for baking a food product, said method comprising treating a flour-containing dough with the protease variant of SEQ ID NO: 9, wherein the treating partially hydrolyzes gluten.

### Pullulanase Variants

Pullulanase, also known as a debranching enzyme, is a specific kind of glucanase that degrades pullulan, a polysaccharide polymer of maltotriose, which is also called glucan. This enzyme is produced by bacteria, such as Klebsiella. Pullulanase is used in processing grain, in the production of glucose and maltose syrup, cyclodextrins, bioethanol, and in the beer and alcohol industry, e.g., to age wine. Pullulanase is added as an anti-staling agent, as well as to improve volume, texture and flavor, in the baking industry.

Pullulanase is active at a temperature range of 40-65° C. and a pH of 4.0-6.5, with an optimum temperature of 60° C. and optimum pH of 4.2-4.6.

The pullulanse variants provided herein are stable under conditions such as high temperature, e.g., over 50° C., pH extremes, e.g., high acidity at pH of 4 or lower, in the presence of salts, alkalis and/or surfactants, and other such severe conditions under which wild-type pullulanse will denature, decompose and/or under which the pullulanse enzymatic function is significantly reduced or eliminated.

The present invention provides a pullulanase variant of a wild-type pullulanase from Bacillus sp. Of SEQ ID NO: 10 comprising mutations:
a) M15Y, I17R, L21T, P23S, A24L, I29G, R35Y, L36M, I40K, T41G, D42R, V47I, R48I, L53K, E54T, A55T, Y59L, V60Y, C61R, V62I, H81N, D97W, Q180P, A204D, T220M, I234T, L242A, D246G, A250S, N251E, S253N, A258D, V260I, E282N, L285Q, A287Q, F297Y, F298N, Q312A, K315V, T316V, I322V, L325F, L334M, F338Y, K343D, P348N, T352L, H360M, E362K, C363D, M365N, P366L, V372C, A383M, C390M, L395A, N399H, V400M, L408M, I410V, L411H, Y418Q, M419I, K420R, A423C, T424D, A426L, G429S, A440N, A450N, P455S, M466W, N474S, H477N, A480D, T481K, L485N, G488N, E489H, Q492E, V494M, M533L, F535A, L537M, E540D, S543D, R544E, K545R, S547A, A559S, I565M, S567A, V578N, Q583N, E597A, T598K, V603F, H604K, S611A, P617S, L621M, R622Q, A625E, L631V, C633F, T635Q, K637N, H639D, I641F, L648N, D649G, V651Y, E653S, I658V, V659C, P665A, W670I, N674D, I676G, S685K, G686K, F687A, E689I, T692I, E693Q, V698K, A699R, I703G, T705G, I707W, Y709L, L710T, and A711V;
b) M15F, I17K, L21T, I29G, T31S, R35Y, E37R, I40K, T41G, D42T, A46T, R48V, L53Y, R76C, L222I, L242A, D246G, N251K, S253N, V260M, E282N, A287Q, F297Y, F298N, A299V, Q312A, K315I, I322V, L325F, F357Y, H360Y, C363D, M365N, V372C, A383M, V392T, L395Q, N399H, L408M, I410V, Y418E, K420R, A423C, T424D, A426I, G429S, I430F, L431M, F433Y, F462H, M466R, H477N, A480D, T481K, G488N, Q492K, A493D, F535A, S543D, R544E, A559S, Q576N, V578D, E597S, S611A, C633F, K637N, L648N, D649H, V651Y, I658V, V659C, I676G, G686K, E689I, N701H, Y709M, and A711Y;
c) V2M, R5K, S7T, V12L, D14K, M15Y, I17R, L21T, I22M, P23S, A24L, I29G, M30K, R35Y, L36M, T38V, I40K, T41G, D42R, V47I, R48I, Y51W, S52E, L53K, E54T, A55D, Y59L, V60Y, C61K, V62I, H81N, D97Y, D98M, E99K, Q180P, A204D, T220M, I234T, L242A, D246G, A250S, N251E, S253N, A258D, V260I, L271M, E282N, K283H, L285Q, A287Q, N293D, F297Y, F298N, Q312A, K315V, T316V, I322V, L325F, Q327E, L334M, F338Y, K343D, P348N, T352L, F357Y, H360M, D361N, C363D, M365N, P366L, V372C, R381K, A383M, A388V, V392K, L395A, E396Q, N399H, V400F, R404K, L408M, I410V, L411H, D414E, V416M, L417N, Y418E, M419I, K420A, E421R, K422E, A423L, T424D, K425S, A426L, P428K, G429S, A440G, A450N, A452Q, P455S, G461A, M466W, V471I, N474S, H477N, A480D, T481K, L485N, G488N, E489H, S490K, Q492E, A493D, V494M, S523C, M533L, F535A, L537M, E540D, S543D, R544E, K545R, S547A, I556V, A559S, I565M, S567A, V578D, Q583N, E597A, T598E, F599Y, K600Y, V603F, H604K, S611A, K614R, A615S, P617S, L621M, R622Q, A625E, L631V, C633F, T635Q, K637N, H639G, L640V, I641L, R644W, L648N, D649N, V651Y, E653S, I658V, V659C, H661C, P665A, D666N, W670I, N674D, I676G, S685K, G686K, F687A, E689I, T692I, E693Q, T697R, V698K, A699Q, I703G, G704A, T705G, I707W, L708V, Y709V, L710T, and A711V;
d) V2M, S7T, D14K, M15Y, I17K, L21T, P23S, I29G, M30K, T31S, R35Y, L36M, E37K, I40K, T41G, D42H, V47I, R48V, L53E, E54T, A55D, V60F, C61K, V62L, R76C, H81N, E99K, H211N, L222I, L242A, D246G, A250S, N251K, S253N, A258S, V260M, G278S, E282N, K283N, A287Q, N293D, F297W, F298N, A299V, H309A, Q312A, K315I, I322V, L325F, H328A, K343I, P348S, F357Y, F358Y, H360Y, C363D, M365N, P366L, V372C, A383M, A388V, V391C, V392T, L395A, N399H, V400F, L408M, I410V, V416M, L417N, Y418E, M419I, K422E, A423C, T424D, A426I, G429S, I430F, L431M, F433Y, L439M, A440G, P444S, A450S, F462H, M466R, H477N, A480D, T481K, G488N, E489G, S490K, A491E, Q492E, A493T, H496A I518V, E522S, M533L, S534A, F535A, E540D, S543E, R544E, K545R, I556V, A559S, S567A, Q576N, V578D, D594H, R596K, F599Y, S611A, L631V, C633F, T635Q, K637N, H639G, I641F, L648N, D649N, V651Y, E653S, I658V, V659C, N674D, I676G, P677T, R679H, S685E, G686K, E689I, T692I, E693Q, K695V, T697R, V698K, N701H, I703G, G704A, and L708V;
e) D14K, M15Y, I17K, I29G, T31S, R35Y, E37K, I40K, T41G, D42H, L45E, A46T, R48V, A55E, R76C, H81N, L222I, L242A, D246G, A250S, N251K, S253N, A258S, V260M, G278S, E282N, A287Q, F297Y, F298N, A299V, Q312A, K315I, I322V, L325F, K343I, P348S, F357Y, F358Y, H360Y, C363D, M365N, P366L, V372C, A383M, V391C, V392T, L395A, N399H, L408M, I410V, V416M, Y418E, M419I, K420R, A423C, T424D, A426I, G429S, I430F, L431M, F433Y, L439M, A440N, F462H, M466R, H477N, A480D, T481K, G488N, A491E, Q492K, A493D, F535A, R544E, K545R, A559S, I565V, S567A, Q576N, V578D, D594H, R596K, E597S, S611A, C633F, T635E, K637N, H639D, I641F, L648N, D649H, V651Y, I658V, V659C, I676G, G686K, E689I, T692I, E693Q, V698K, N701H, I703G, Y709M, L710M, and A711Y;
f) M15Y, I17K, L21T, P23S, A24L, I29G, T31S, R35Y, I40K, T41G, R48I, L53Y, V60F, R76C, L222I, L242A, D246G, N251K, S253N, V260M, G278S, E282N, A287Q, F297Y, F298N, A299V, Q312A, K315I, I322V, L325F, F338Y, K343I, F357Y, H360Y, C363D, M365N, V372C, A383M, V391C, V392T, L395Q, N399H, L408M, I410V, Y418E, K420R, A423C, T424D, A426I, G429S, I430F, L431M, F433Y, F462H, M466R, H477N, A480D, T481K, G488N, Q492K, A493D, F535A, S543D, R544E, A559S, Q576N, V578D, S611A, C633F, K637N, L648N, D649H, V651Y, I658V, V659C, I676G, G686K, E689I, N701H, Y709M, and A711Y;
g) R5K, M15Y, I17K, L21T, I22M, P23S, A24L, I29G, R35Y, I40K, T41G, D42R, L45K, R48V, L53H, E54T, A55E, Y59L, V60Y, V62I, D97W, Q180P, T220M, I234T, L242A, D246G, A250S, N251E, S253N, A258D, V260I, E282N, L285Q, A287Q, F297Y, F298N, A299V, Q312A, K315V, T316V, I322V, L325F, L334M, F338Y, K343S, P348N, T352L, E362K, C363D, M365N, P366L, V372C, A383M, A388V, V392L, L395A, N399H, L408M, I410V, V416M, Y418Q, M419I, K420A, A423C, T424D, K425S, A426L, G429S, A450N, P455S, M466W, N474S, H477N, A480D, T481K, L485N, A491F, V494M, F535A, S543E, R544D, I565M, V578N, Q583N, E597A, T598K, V603F, S611A, P617S, R622Q, A625E, L631V, C633F, K637N, H639D, I641F, L648N, D649N, V651Y, E653S, V659C, P665A, D666H, I676G, S685K, G686K, F687A, E689I, T692I, E693Q, V698K, A699R, I703G, Y709V, L710M, and A711V;
h) V2M, S7T, D14K, M15Y, I17K, L21T, I29G, M30K, T31S, R35Y, L36M, E37K, I40K, T41G, D42N, A46T, V47I, R48V, L53H, E54T, A55D, C61K, V62L, R76C, H81N, E99K, H211N, L222I, L242A, D246G, A250S, N251K, S253N, S255T, A258S, V260M, G278S, E282N, K283N, A287Q, N293D, F297W, F298N, A299V, H309A, Q312A, K315I, I322V, L325F, H328A, K343I, P348S, F357Y, F358Y, H360Y, C363D, M365N, P366L, V372C, A383M, A388V, V392T, L395A, N399H, V400F, L408M, I410V, V416M, L417N, Y418E, M419I, K422E, A423C, T424D, A426I, G429S, I430F, L431M, F433Y, L439M, A440G, P444S, A450S, F462H, M466R, H477N, A480D, T481K, G488N, E489G, S490K, A491E, Q492E, A493T, H496A, G497A, I518V, E522S, M533L, S534A, F535A, E540D, S543E, R544E, K545R, I556V, A559S, S567A, Q576N, V578D, D594H, R596K, F599Y, S611A, L631V, C633F, T635Q, K637N, H639D, I641F, L648N, D649N, V651Y, E653S, I658V, V659C, N674D, I676G, P677T, R679H, S685E, G686K, E689I, T692I, E693Q, K695V, T697R, V698K, N701H, I703G, G704A, and L708V;
i) D14K, I17K, L21T, P23S, A24L, I29G, T31S, R35Y, E37K, I40K, T41G, D42H, L45E, R48I, L53Y, A55D, V60F, C61R, R76C, H81N, L222I, L242A, D246G, A250S, N251K, S253N, S255T, A258S, V260M, G278S, E282N, A287Q, F297Y, F298N, A299V, Q312A, K315I, I322V, L325F, K343I, P348S, F357Y, H360Y, C363D, M365N, P366L, V372C, A383M, V391C, V392T, L395Q, N399H, L408M, I410V, Y418E, M419I, K420R, A423C, T424D, A426I, G429S, I430F, L431M, F433Y, L439M, A440N, A450C, F462H, M466R, H477N, A480D, T481K, L485T, G488N, A491E, Q492K, A493D, F535A, S543E, R544E, K545R, A559S, S567A, Q576N, V578D, D594H, R596K, E597S, S611A, C633F, T635E, K637N, H639D, I641F, L648N, D649N, V651Y, I658V, V659C, I676G, G686K, E689I, T692I, E693Q, V698K, N701H, I703G, L710M, and A711I;
j) D14K, M15Y, I17K, L21T, P23S, I29G, T31S, R35Y, E37K, I40K, T41G, D42H, L45E, R48I, L53N, A55D, V60F, C61R, R76C, H81N, L222I, L242A, D246G, A250S, N251K, S253N, A258S, V260M, E282N, K283G, A287Q, F297Y, F298N, A299V, H309A, Q312A, K315I, I322V, L325F, P348S, F357Y, F358Y, H360Y, C363D, M365N, P366L, V372C, A383M, V391M, V392T, L395Q, N399H, V400F, L408M, I410V, V416M, L417N, Y418Q, M419I, K420R, A423C, T424S, A426I, G429S, I430F, L431M, F433Y, L439M, A440G, A450C, M466R, H477N, A480D, T481K, L485T, G488E, E489G, A491E, Q492E, A493T, H496A, I518V, E522S, M533L, F535A, E540D, S543E, R544E, K545R, A559S, I565M, S567A, Q576N, V578D, D594H, R596K, S611A, C633F, T635Q, K637N, H639D, I641F, L648N, D649N, V651Y, E653S, I658V, V659C, I676G, P677T, R679H, G686K, T692I, E693Q, T697R, V698K, N701H, I703G, L708V, L710M, and A711Y;
k) R5K, M15Y, I17K, I29G, I40K, T41G, D42H, R48V, L53H, D97H, Q180P, T220M, I234T, L242A, D246G, A250N, N251E, S253N, A258D, V260I, G278S, E282N, L285Q, A287Q, H296Q, F297Y, F298N, Q312A, K315V, T316V, I322V, L325F, L334M, F338Y, P348S, T352L, C363D, M365N, P366L, V372C, A383M, A388V, C390M, N399H, L408M, I410V, L411H, V416M, Y418E, M419I, K420A, A423C, T424D, K425S, A426L, A450N, P455S, M466W, N474S, H477N, A480D, T481K, A491F, V494M, S523C, F535A, S543E, R544E, I565M, V578D, Q583N, T598E, V603F, S611A, K614R, R622Q, A625E, L631V, C633F, K637N, I641F, L648N, D649N, V651Y, I658M, V659C, P665A, D666H, I676G, S685K, G686K, E689T, A699R, I703G, Y709V, L710T, and A711V;
l) V2M, R5K, S7T, D14K, M15Y, I17R, L21T, I22M, P23S, A24L, I29G, M30K, R35Y, L36M, T38V, I40K, T41G, D42R, V47I, R48I, Y51W, S52E, L53K, E54T, A55D, Y59L, V60Y, C61K, V62I, H81N, D97Y, D98M, E99K, Q180P, A204D, T220M, I234T, L242A, D246G, A250S, N251E, S253N, A258D, V260I, L271M, G278S, E282N, K283G, L285Q, A287Q, N293D, H296Q, F297Y, F298N, Q312A, K315V, T316V, I322V, L325F, Q327E, L334M, F338Y, K343D, P348H, T352L, F357Y, H360M, D361N, C363D, M365N, P366Y, V372C, R381K, A383M, V392I, L395A, N399H, V400M, L408M, I410V, L411H, D414E, V416M, L417N, Y418Q, M419I, K420R, K422E, A423C, T424D, K425S, A426L, G429S, A440G, A450N, A452K, P455S, G461A, M466W, V471I, N474S, H477N, A480D, T481K, L485N, G488N, E489H, S490K, Q492E, A493D, V494M, S523C, M533L, F535A, L537M, E540D, S543E, R544D, K545R, S547Q, I556V, A559S, I565M, S567A, V578D, Q583N, E597A, T598K, F599Y, H604K, S611A, A615S, P617S, L621M, R622Q, A625E, L631V, C633F, T635Q, K637N, H639D, I641F, R644W, L648N, D649G, V651Y, E653S, I658V, V659C, P665A, D666N, W670I, N674D, I676E, S685K, G686K, F687A, E689I, T692I, E693Q, T697R, V698K, A699Q, I703G, G704A, T705G, I707W, L708V, Y709V, L710M, and A711V;
m) R5K, M15Y, L21T, P23S, A24L, I29G, R35Y, E37R, T41G, D42T, P44T, R48I, L53H, V60Y, V62I, Q180P, T220M, L242A, A250N, N251E, S253N, A258D, V260I, G278S, E282N, L285Q, A287Q, H296Q, F297Y, F298N, Q312A, K315V, T316V, I322V, L325F, F338Y, P348H, T352L, E362K, C363D, M365H, V372C, A383M, C390M, N399H, L408M, I410V, L411H, V416M, Y418Q, M419I, A423L, T424D, A426I, P428R, A450N, P455S, H477N, A480D, A491F, V494M, S523C, F535A, S543E, S547Q, I565M, V578D, Q583N, E597A, T598E, H604K, S611A, K614R, R622Q, L631V, C633F, K637N, I641F, L648N, V651Y, V659C, P665A, D666H, I676G, S685K, F687A, E689I, A699R, I703G, Y709V, L710M, and A711V;
n) R5K, M15Y, L21T, P23S, I29G, R35Y, E37R, T41G, D42T, P44T, R48I, L53H, V60Y, V62I, D97W, Q180P, T220M, L242A, A250N, N251E, S253N, A258D, V260I, E282N, L285Q, A287Q, F297Y, F298N, Q312A, K315V, T316V, I322V, L325F, L334M, F338Y, P348H, T352L, F357Y, E362K, C363D, M365H, V372C, A383M, C390M, N399H, L408M, I410V, L411H, V416M, Y418Q, M419I, A423L, T424D, A450N, P455S, N474S, H477N, A480D, V494M, F535A, S543E, I565M, V578D, Q583N, T598K, V603F, S611A, K614R, R622Q, L631V, C633F, K637N, L648N, V651Y, V659C, H661C, I676G, S685K, F687A, E689I, A699R, I703G, Y709V, L710M, and A711V;
o) M15Y, L21T, I29G, T31S, R35Y, E37R, I40K, T41G, R48I, R76C, L222I, L242A, D246G, N251K, S253N, V260M, G278S, E282N, A287T, F298N, A299V, Q312A, K315I, I322V, L325F, K343I, F357Y, H360Y, C363D, M365N, V372C, A383M, A388I, V392C, L395Q, N399H, L408M, Y418E, K420R, A423C, T424D, A426I, G429S, I430F, L431M, F433Y, F462H, M466R, H477N, L485T, A493D, F535A, S543D, R544E, A559S, V578D, E597S, S611A, C633F, K637N, L648N, D649H, V651Y, I658V, V659C, I676G, N701H, Y709M, and A711Y;
p) R5K, V12L, M15Y, I17K, L21T, I22M, P23S, I29G, R35H, L36M, I40K, T41G, D42R, R48V, Y51W, L53H, E54T, A55D, Y59L, H81N, D97W, L146M, Q180P, T220M, I234T, L242A, D246G, A250S, N251E, S253N, A258D, V260I, G278S, E282N, L285Q, A287Q, H296Q, F297Y, F298N, Q312A, K315V, T316V, I322V, L325F, L334M, F338Y, K343S, P348N, T352L, H360M, C363D, M365N, P366L, V372C, A383M, L395A, N399H, V400M, L408M, I410V, L411H, Y418Q, M419I, K420R, A423C, T424D, K425S, A426L, G429S, A440N, A450N, P455S, M466W, N474S, H477N, A480D, T481K, L485N, G488N, E489H, Q492E, V494M, S523C, M533L, F535A, L537M, S543D, R544D, K545R, A559S, I565M, S567A, V578N, Q583N, E597A, T598K, V603F, H604K, S611A, R622Q, A625E, L631V, C633F, T635Q, K637N, H639D, L640V, L648N, D649N, V651Y, E653S, I658V, V659C, H661C, P665A, D666H, W670I, I676G, S685K, G686K, F687A, E689I, T692I, E693Q, V698K, A699R, I703G, Y709V, L710T, and A711V;
q) R5K, M15Y, I17K, I29G, R35Y, T41G, D42H, R48I, L53H, D97W, Q180P, T220M, L242A, A250N, N251E, S253N, A258D, V260I, E282N, L285Q, A287Q, F297Y, F298N, Q312A, K315V, T316V, I322V, L325F, L334M, F338Y, P348H, T352L, E362K, C363D, M365H, V372C, A383M, A388V, N399H, L408M, I410V, L411H, V416M, Y418Q, M419I, A423L, T424D, A426I, P428R, A450N, P455S, N474S, H477N, A480D, A491F, V494M, S523C, F535A, S543E, R544D, I565M, V578D, Q583N, E597A, T598E, V603F, H604K, S611A, R622Q, A625E, L631V, C633F, K637N, I641F, L648N, D649N, V651Y, I658M, V659C, P665A, D666H, I676G, S685K, F687A, E689I, A699R, I703G, Y709V, L710T, and A711V;
r) M15Y, L21T, P23S, I29G, T31S, R35Y, T41G, R48I, L53Y, R76C, L222I, L242A, N251K, S253N, V260M, G278S, E282N, A287T, F298N, A299V, Q312A, K315I, I322V, L325F, K343I, F357Y, F358Y, H360Y, C363D, M365N, V372C, A383M, A388I, V392T, L395Q, N399H, L408M, Y418E, K420R, A423C, T424D, A426I, I430F, L431M, M466R, L485T, A493D, F535I, S543D, R544E, V578D, E597S, S611A, K637N, L648N, D649H, V651Y, I658V, V659C, I676G, and A711I;
s) M15Y, L21T, P23S, I29G, T31S, R35Y, I40K, T41G, R48I, L53Y, R76C, L222I, L242A, D246G, S253N, V260M, G278S, E282N, A287T, F298N, A299V, Q312A, K315I, I322V, L325F, K343I, F357Y, H360Y, C363D, M365N, V372C, A383M, V392T, L395Q, N399H, L408M, Y418E, K420R, A423C, T424D, A426I, G429S, I430F, L431M, F433Y, F462H, M466R, H477N, L485T, A493D, F535A, R544E, A559S, V578D, S611A, C633F, K637N, L648N, D649H, V651Y, I658V, V659C, I676G, N701H, Y709M, and A711Y;
t) R5K, M15Y, I17K, I29G, I40K, T41G, D42H, L45K, R48V, L53H, D97W, Q180P, T220M, I234T, L242A, D246G, N251E, S253N, A258D, V260I, E282N, L285Q, A287Q, F297Y, F298N, Q312A, K315V, T316V, I322V, L325F, L334M, F338Y, P348S, T352L, E362K, C363D, M365N, P366L, V372C, A383M, A388V, V392L, N399H, L408M, I410V, V416M, Y418Q, M419I, K420A, A423C, T424D, K425S, A426L, A450N, P455S, M466W, H477N, A480D, T481K, V494M, F535A, S543E, R544D, I565M, V578N, Q583N, E597A, T598E, H604K, S611A, K614R, R622Q, A625E, L631V, C633F, K637N, I641F, L648N, D649N, V651Y, I658M, V659C, P665A, D666H, I676G, S685K, G686K, E689T, A699R, I703G, Y709V, L710T, and A711V;
u) R5K, V12L, M15Y, I17K, L21T, I22M, P23S, I29G, R35Y, L36M, I40K, T41G, D42R, R48I, Y51W, L53H, E54T, A55D, Y59L, V60Y, V62I, H81N, D97W, T220M, I234T, L242A, D246G, A250S, N251E, S253N, A258D, V260I, E282N, L285Q, A287Q, F297Y, F298N, A299V, Q312A, K315V, T316V, I322V, L325F, L334M, F338Y, K343S, P348N, T352L, H360M, C363D, M365N, P366L, V372C, A383M, A388V, V392L, L395A, N399H, V400M, L408M, I410V, L411H, V416M, Y418Q, M419I, K420R, E421R, A423C, T424D, K425S, A426L, G429S, A440N, A450N, P455S, G461H, M466W, N474S, H477N, A480D, T481K, L485N, G488N, E489H, Q492E, V494M, S523C, M533L, F535A, L537M, S543D, R544E, K545R, L558T, A559S, I565M, S567A, V578N, Q583N, E597A, T598E, V603F, H604K, S611A, P617S, R622Q, A625E, L631V, C633F, T635Q, K637N, H639D, I641F, L648N, D649N, V651Y, E653S, I658V, V659C, P665A, D666H, W670I, I676G, S685K, G686K, F687A, E689I, T692I, E693Q, V698K, A699R, I703G, Y709V, L710M, and A711V;
v) M15Y, I17K, L21T, P23S, A24L, I29G, T31S, R35Y, E37K, I40K, T41G, D42H, R48I, L53Y, V60F, R76C, H81N, L222I, L242A, D246G, S253N, A258S, V260M, G278S, E282N, A287Q, F297W, F298N, A299V, Q312A, K315I, I322V, L325F, K343I, F357Y, H360Y, C363D, M365N, P366L, V372C, A383M, A388I, V392T, L395A, N399H, L408M, I410V, V416M, Y418E, A423C, T424D, A426I, G429S, I430F, L431M, F433Y, A440N, F462H, M466R, H477N, A480D, T481K, L485T, G488N, Q492K, A493D, F535A, S543D, R544E, A559S, I565V, Q576N, V578D, D594H, R596K, S611A, C633F, T635E, K637N, H639D, L648N, D649H, V651Y, I658V, V659C, I676G, G686K, E689I, T692I, V698K, L710M, and A711Y;
w) M15Y, I17K, L21T, P23S, A24L, I29G, R35Y, I40K, T41G, D42R, L45K, R48V, L53H, E54T, A55E, Y59L, V60Y, V62I, D97H, Q180P, T220M, I234T, L242A, D246G, A250S, N251E, S253N, A258D, V260I, E282N, L285Q, A287Q, F297Y, F298N, Q312A, K315V, T316V, I322V, L325F, L334M, F338Y, K343S, P348N, T352L, C363D, M365N, P366L, V372C, A383M, A388I, V392L, L395A, N399H, L408M, I410V, L411H, V416M, Y418Q, M419I, K420A, A423C, T424D, K425S, A426L, G429S, I430L, A450N, P455S, M466W, N474S, H477N, A480D, T481K, L485N, A491F, V494M, F535A, S543E, R544D, S547Q, I565M, V578N, Q583N, E597A, T598E, H604K, S611A, R622Q, A625E, L631V, C633F, K637N, H639D, I641F, L648N, D649N, V651Y, E653S, V659C, P665A, D666H, I676G, S685K, G686K, F687A, E689I, T692I, E693Q, V698K, A699R, V700L, I703G, Y709V, L710T, and A711V;
x) R5K, M15Y, I17K, L21T, P23S, A24L, I29G, R35Y, E37R, T41G, D42H, P44T, R48I, L53H, V60Y, V62I, D97W, L146M, Q180P, T220M, L242A, A250N, N251E, S253N, A258D, V 260I, E282N, L285Q, A287Q, F297Y, F298N, Q312A, K315V, T316V, I322V, L325V, L334M, F338Y, P348H, T352L, C363D, M365N, V372C, A383M, C390M, N399H, L408M, I410V, L411H, V416M, Y418Q, M419I, A423L, T424D, A426I, P428R, A450N, P455S, N474S, H477N, A480D, A491F, V494M, F535A, S543E, R544D, I565M, V578D, Q583N, E597A, T598E, V603F, H604K, S611A, K614R, R622Q, A625E, L631V, C633F, K637N, I641F, L648N, D649N, V651Y, V659C, P665A, D666H, I676G, S685K, F687A, E689I, A699R, I703G, Y709V, L710M, and A711V;
y) R5K, M15Y, I17R, L21T, P23S, I29G, R35Y, L36M, I40K, T41G, D42R, V47I, R48I, Y51W, L53K, E54T, A55T, Y59L, V60Y, C61R, V62I, H81N, D97H, Q180P, A204D, T220M, I234T, L242A, D246G, A250S, N251E, S253N, A258D, V260I, G278S, E282N, L285Q, A287Q, H296Q, F297Y, F298N, A299V, Q312A, K315V, T316V, I322V, L325F, L334M, F338Y, K343S, P348N, T352L, H360M, C363D, M365N, P366L, V372C, A383M, A388V, L395A, N399H, V400M, L408M, I410V, L411H, Y418Q, M419I, K420A, A423C, T424D, K425S, A426L, G429S, A440N, A450N, P455S, M466W, N474S, H477N, A480D, T481K, L485N, G488E, E489H, Q492E, V494M, M533L, F535A, L537M, E540D, S543E, R544D, K545R, S547A, A559S, I565M, S567A, V578D, Q583N, E597A, T598E, H604K, S611A, A615S, L621M, R622Q, A625E, L631V, C633F, T635Q, K637E, H639D, I641F, A642G, L648N, D649G, V651Y, E653S, I658M, V659C, W670I, N674D, I676G, S685K, G686K, F687A, E689I, T692I, E693Q, V698K, A699R, I703G, T705G, I707W, Y709V, L710M, and A711V;
z) M15Y, L21T, I29G, T31S, R35Y, T41G, R48I, R76C, L222I, L242A, S253N, V260M, G278S, E282N, A287T, F298N, A299V, Q312A, K315I, I322V, L325F, K343I, F357Y, H360Y, C363D, M365N, V372C, A383M, A388I, V392T, L395Q, N399H, L408M, Y418E, K420R, A423C, T424D, A426I, I430F, L431M, F462H, M466R, G488N, Q492K, A493D, F535I, S543D, R544E, V578D, S611A, K637N, L648N, D649H, V651Y, I658V, V659C, I676G, E689I, N701H, Y709M, and A711I;
aa) D14K, M15Y, I17K, I29G, T31S, R35Y, E37K, I40K, T41G, D42H, L45E, A46T, R48V, L53N, A55E, R76C, H81N, L222I, L242A, D246G, A250S, N251K, S253N, A258S, V260M, G278S, E282N, K283G, A287Q, F297Y, F298N, A299V, H309A, Q312A, K315I, I322V, L325F, K343I, P348S, F357Y, H360Y, C363D, M365N, P366L, V372C, A383M, A388V, V392T, L395Q, N399H, V400F, L408M, I410V, V416M, L417N, Y418Q, M419I, K420R, A423C, T424D, A426I, G429S, I430F, L431M, F433Y, L439M, A440G, A450C, M466R, H477N, A480D, T481K, G488N, E489G, A491E, Q492E, A493T, H496A, I518V, E522S, M533L, F535A, E540D, S543D, R544E, K545R, A559S, S567A, Q576N, V578D, D594H, R596K, S611A, C633F, T635Q, K637D, H639D, I641F, L648N, D649N, V651Y, E653S, I658V, V659C, I676G, P677T, R679H, G686K, E689I, T692I, E693Q, T697R, V698K, N701H, I703G, L708V, L710M, and A711Y; or
bb) I17K, I29G, T31S, R35Y, E37K, I40K, T41G, D42H, A46T, R48V, L53Y, R76C, H81N, L222I, L242A, D246G, S253N, A258S, V260M, G278S, E282N, A287Q, F297Y, F298N, A299V, Q312A, K315I, I322V, L325F, K343I, F357Y, F358Y, H360Y, C363D, M365N, P366L, V372C, A383M, V391C, V392T, L395Q, N399H, L408M, I410V, V416M, Y418E, K420R, A423C, T424S, A426I, G429S, I430F, L431M, F433Y, A440N, F462H, M466R, H477N, A480D, T481K, G488N, Q492K, A493D, F535A, R544E, A559S, Q576N, V578D, D594H, R596K, S611A, C633F, T635E, K637N, H639D, L648N, D649H, V651Y, I658V, V659C, I676G, G686K, E689I, T692I, V698K, L710M, and A711Y.

The present invention also provides a pullulanase variant of a wild-type pullulanase from Klebsiella pneumoniae of SEQ ID NO: 11 comprising mutations:
a) V66A, R71N, D105C, T122A, I142C, S225N, D230N, N232E, T236D, T246S, V247L, Q250E, V251Q, S252A, M253E, R254K, P262A, K265R, V270T, N271D, S284A, E286N, V302A, A313S, R328H, E338V, V341I, A344T, K347Q, V349I, S358N, A362S, K372H, A374D, M380V, K389N, G424N, G427N, K435N, T444A, A462E, E463D, L464W, Q474D, E475N, N477A, V490T, I493V, E523C, E532S, G535S, D538C, V543I, K556S, T566A, L567A, Q570A, T571N, Y574F, F581Y, P594A, T597G, T605Q, I607V, D613N, P632Y, T653V, S656G, A660N, S664C, I677M, V683T, T685A, D687E, I690F, Y700H, Q705N, S708E, W710R, L771Q, Q773S, P785A, E787D, T789S, L791T, D793Q, L805A, D813S, F814Y, L816F, I817T, K819N, A822N, V823E, K824V, K825R, G826A, S827A, E828Q, I829L, A834Q, G836A, V845A, M860A, I861L, S862A, A865T, E868D, A869T, L871M, T873D, A879T, S881A, L882A, V885A, Q896H, V931K, R935G, I945L, A954V, E958Q, L961M, K962Q, M964A, T965A, D985S, A987R, T988E, T1000A, T1004Q, M1015S, Q1016R, S1020D, A1031V, T1047N, Q1069R, A1072E, E1087V, E1092G, and A1096T;
b) V66A, R71N, D105C, T122A, I142C, S225N, D230N, N232E, T236D, T246S, V247L, Q250E, V251Q, S252A, R254K, P262A, K265R, V270T, N271D, S284A, E286N, V302A, R328H, E338V, V341I, A344T, K347Q, V349I, S358N, A362S, D370E, K372H, A374D, M380V, K389N, G424N, G427N, T429I, K435N, T444A, A462E, E463D, L464W, Q474D, E475N, N477A, V490T, I493V, E523C, E532S, G535S, D538C, V543I, Q552K, K556S, T566A, L567A, Q570A, T571N, Y574F, F581Y, P594A, T597G, K601A, T605Q, I607V, D613N, P632Y, T653V, S656G, A660N, S664C, I677M, V683T, T685A, D687E, I690F, Y700H, Q705N, S708E, W710R, L771Q, Q773S, P785A, E787D, T789S, L791T, D793Q, L805A, D813S, F814Y, L816F, I817T, K819N, A822N, V823E, K824V, K825R, G826S, S827A, E828Q, I829L, A834Q, G836A, V845A, M860A, I861L, S862A, A865T, E868D, A869T, L871M, T873D, A879T, S881A, L882A, V885A, Q896H, V931K, R935G, I945L, E958Q, L961M, K962Q, M964A, T965A, D985S, A987R, T988E, T998A, T1000A, T1004Q, M1015S, Q1016R, S1020D, A1031V, T1047N, Q1069R, A1072E, E1087V, E1092G, and A1096T;
c) D105C, T122A, I142C, S225N, D230N, T236D, T246S, Q250E, V251Q, S252A, M253E, R254K, P262A, V270T, N271D, S284A, V302A, E338V, V341I, A344T, V349I, S358N, K372H, A374K, M380V, Q386Y, G424N, G427S, K435N, A462E, L464W, Q474D, E475N, N477A, V490T, I493V, E523C, E532S, D538C, K556S, T566A, L567Q, Q570A, Y574F, P594A, T597G, K601A, T605Q, D613N, P632Y, T653V, S656G, A660N, S664C, T685A, I690F, Y700H, Q705N, S708E, W710R, L771Q, Q773S, E787D, T789S, D793Q, L805A, L816F, I817T, K819S, A822N, V823E, K824V, K825R, G826A, S827A, E828Q, I829L, A834Q, G836A, M860A, S862A, E868D, A869T, T873D, L882A, Q896H, V931K, R935G, I945L, A954V, E958Q, L961M, K962Q, M964A, D985S, T988E, T1000A, T1004Q, M1015S, Q1016R, S1020D, A1031V, T1047N, E1087V, E1092G, and A1096T;
d) D105C, I142C, T236S, S284T, V341I, V349I, E523C, I524V, D538C, S664C, Q705H, K825R, and T1000A;
e) D105C, I142C, S225N, T236D, T246S, S252A, P262A, N271D, S284A, V341I, A344T, V349I, S358N, K435N, A462E, L464W, Q474D, N477A, V490T, E523C, I524V, D538C, Q552K, K556S, L567A, Q570A, Y574F, P594A, T597G, P632Y, S656G, S664C, T685A, I690F, Y700H, Q705N, L707N, S708E, W710R, L771Q, Q773S, D793Q, L805A, L816F, I817T, K819S, A822N, V823E, K824V, K825R, S827A, I829L, G836A, M860I, S862A, T873D, L882A, R935G, E958Q, L961M, K962Q, D985S, T988E, T1000A, M1015S, S1020D, A1031V, E1087V, and E1092G;
f) D105C, I142C, S225N, D230N, T236D, T246S, V251Q, S252A, P262A, K265R, V270K, N271D, S284A, Q336N, V341I, A344T, V349I, S358N, K435N, A462E, L464W, Q474D, E475N, N477A, V490T, E523C, I524V, E532S, D538C, Q552K, K556S, T566A, L567Q, Q570A, Y574F, D593N, P594A, T597G, P632Y, S656G, A660N, S664C, T685A, I690F, Y700H, Q705N, L707N, S708E, W710R, L771Q, Q773S, T789S, D793Q, L805A, L816F, I817T, K819S, A822N, V823E, K824V, K825R, S827A, I829L, A834T, G836A, M860A, S862A, A869T, T873D, L882A, R935G, E958Q, L961M, K962Q, M964A, D985S, T988E, T1000A, M1015S, S1020D, A1031V, E1087V, and E1092G;
g) D105C, T122A, I142C, S225N, D230N, T236D, T246S, Q250E, V251Q, S252A, M253E, R254K, P262A, K265R, V270T, N271D, S284A, V302A, Q336N, E338V, V341I, A344T, V349I, S358N, K372H, A374K, M380V, G424N, G427S, K435N, A462E, L464W, Q474D, E475N, N477A, V490T, I493V, E523C, E532S, D538C, K556S, T566A, L567A, Q570A, Y574F, P594A, T597G, K601T, T605Q, D613N, P632Y, T653V, S656G, A660N, S664C, T685A, I690F, Y700H, Q705N, L707N, S708E, W710R, L771Q, Q773S, E787D, T789S, D793Q, L805A, L816F, I817T, K819S, A822N, V823E, K824V, K825R, G826A, S827A, E828Q, I829L, A834Q, G836A, M860A, S862A, E868D, A869T, T873D, L882A, Q896H, V931K, R935G, I945L, A954V, E958Q, L961M, K962Q, M964A, D985S, T988E, T1000A, T1004Q, M1015S, Q1016R, S1020D, A1031V, T1047N, E1087V, E1092G, and A1096T;
h) D105C, T122S, I142C, S225N, D230N, T236D, T246S, V251Q, S252A, P262A, V270T, N271D, S284A, E338V, V341I, A344T, V349I, S358N, M380L, G427S, K435N, A462E, L464W, Q474D, E475N, N477A, V490T, E523C, E532S, D538C, Q552K, K556S, T566A, L567Q, Q570A, Y574F, P594A, T597G, T605A, P632Y, S656G, A660N, S664C, T685A, I690F, Y700H, Q705N, S708E, W710R, L771Q, Q773S, E787D, T789S, D793Q, L805A, L816F, I817T, K819S, A822N, V823E, K824V, K825R, S827A, I829L, A834T, G836A, M860A, S862A, E868D, A869T, T873D, L882A, V931K, R935G, A954V, E958Q, L961M, K962Q, M964A, D985S, T988E, T1000A, M1015S, Q1016R, S1020D, A1031V, T1047N, E1087V, and E1092G;
i) D105C, I142C, S225N, T236S, S252A, P262A, S284A, V341I, V349I, K435N, A462E, N477A, E523C, I524V, D538C, L567A, Q570G, Y574F, P594A, T597G, P632Y, S656G, S664C, T685A, I690F, Q705N, L707N, S708E, Q773S, D793Q, L805A, K819S, V823E, K824V, K825R, S827A, G836A, R935G, L961M, K962Q, D985S, T988E, T1000A, M1015S, S1020D, and A1031V;
j) D105C, I142C, S225N, T236S, T246S, S252A, P262A, N271D, S284A, V341I, V349I, K435N, A462E, L464W, Q474N, N477A, V490T, E523C, I524V, D538C, Q552K, L567Q, Y574F, P594A, T597G, P632Y, S656G, S664C, T685A, I690F, Y700H, Q705N, L707N, S708E, L771Q, Q773S, D793Q, L805A, K819S, V823E, K824V, K825R, S827A, G836A, M860I, S862A, L882A, R935G, L961M, K962Q, D985S, T988E, T1000A, M1015S, S1020D, A1031V, and E1087V;
k) D105C, I142C, S225N, D230N, T236D, T246S, V251Q, S252A, P262A, V270K, N271D, S284A, V341I, A344T, V349I, S358N, K435N, A462E, L464W, Q474D, E475N, N477A, V490T, E523C, I524V, E532S, D538C, K556S, T566A, L567Q, Q570A, Y574F, P594A, T597G, K601A, P632Y, S656G, A660N, S664C, T685A, I690F, Y700H, Q705N, S708E, W710R, L771Q, Q773S, T789S, D793Q, L805A, L816F, I817T, K819S, A822N, V823E, K824V, K825R, S827A, I829L, A834T, G836A, M860A, S862A, A869T, T873D, L882A, R935G, E958Q, L961M, K962Q, M964A, D985S, T988E, T1000A, M1015S, S1020D, A1031V, E1087V, and E1092G;
l) D105C, T122S, I142C, S225N, D230N, T236D, T246S, V251Q, S252A, P262A, V270T, N271D, S284A, Q336N, E338V, V341I, A344T, V349I, S358N, M380L, G427S, K435N, A462E, L464W, Q474D, E475N, N477A, V490T, E523C, E532S, D538C, K556S, T566A, L567A, Q570A, Y574F, P594A, T597G, T605A, P632Y, S656G, A660N, S664C, T685A, I690F, Y700H, Q705N, L707N, S708E, W710R, L771Q, Q773S, E787D, T789S, D793Q, L805A, L816F, I817T, K819S, A822N, V823E, K824V, K825R, S827A, I829L, A834T, G836A, M860A, S862A, E868D, A869T, T873D, L882A, V931K, R935G, E958Q, L961M, K962Q, M964A, D985S, T988E, T1000A, M1015S, Q1016R, S1020D, A1031V, T1047N, E1087V, and E1092G; or
m) D105C, I142C, S225N, T236S, S252A, P262A, S284A, V341I, V349I, K435N, A462E, N477A, E523C, I524V, D538C, Q552K, L567A, Q570G, Y574F, P594A, T597G, P632Y, S656G, S664C, T685A, I690F, Q705N, L707N, S708A, Q773S, D793Q, L805A, K819S, V823E, K824V, K825R, S827A, G836A, R935G, L961M, K962Q, D985S, T988E, T1000A, M1015S, S1020D, and A1031V.

In an embodiment, the present invention provides a nucleic acid sequence encoding the above-described pullulanase variant of any one of SEQ ID NO: 10 or SEQ ID NO: 11.

In another embodiment of the present invention, an expression vector comprises the nucleic acid sequence encoding the pullulanase variant of any one of SEQ ID NO: 10 or SEQ ID NO: 11.

In an embodiment, the present invention provides a host cell transformed with the expression vector comprising the nucleic acid sequence encoding the pullulanase variant of any one of SEQ ID NO: 10 or SEQ ID NO: 11.

In a further embodiment, the present invention provides a method for producing a pullulanase variant, said method comprising expressing the pullulanase variant in the transformed host cell, wherein the host cell is transformed with the expression vector comprising the nucleic acid sequence encoding the pullulanase variant of any one of SEQ ID NO: 10 or SEQ ID NO: 11.

In an embodiment, the present invention provides a grain processing aid comprising the pullulanase variant of any one of SEQ ID NO: 10 or SEQ ID NO: 11.

In another embodiment, the present invention provides a method for producing fermentable sugars having an increased glucose concentration, said method comprising:

a) gelatinizing a polysaccharide with an a-amylase to produce maltodextrin; and

b) treating the maltodextrin with the pullulanase variant of any one of claim 62 or 63, thereby producing fermentable sugars having an increased glucose concentration compare to fermentable sugars which has not been treated with the pullulanase.

In an embodiment of the above-described method for producing fermentable sugars having an increased glucose concentration, the polysaccharide is a starch, an amylopectin, a pullulan or an oligosaccharide.

### Transglutaminase Variants

Transglutaminase is an enzyme that catalyzes the formation of isopeptide bonds between a free amine group, such as a protein- or peptide-bund lysine and an acyl group at the end of a side chain of protein- or peptide-bound glutamine and the bonds form cross-linked protein polymers that are insoluble and are highly resistant to proeteolysis. Several transglutaminase are found in humans, including Factor XIII (fibrin stabilizing factor) that crosslinks fibrin in blood coagulation, whose deficiency predisposes a person to hemorrhage; tissue transglutaminase; and keratinocyte transglutaminase, among others.

Transglutaminase is used in food processing as a meat glue to bond proteins together in foods, such as meat, fish, imitation crabmeat, processes meat or processed fish, e.g., boneless fish. It also is used to improve the texture of protein-rich foods, including meat or fish pastes. One transglutaminase, tissue transglutaminase, may be used as a surgical glue and also is being studies for anti-tumor activity.

The transglutaminase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type transglutaminase will denature, decompose and/or under which the transglutaminase enzymatic function is significantly reduced or eliminated.

The present invention provides a transglutaminase variant of a wild-type transglutaminase from Streptomyces mobaraensis of SEQ ID NO: 12 comprising mutations:
a) P98A, S102R, K128A, Q153P, G236K, K260N, D261N, S283G, S325G, D379E, S382A, A388T, V391I, V394I, and T406K;
b) D54A, P98A, S102R, K128A, Q153P, G176T, Q203E, A234G, G236K, K260N, S283G, S325G, D379E, S382A, A388T, V390I, V391I, V394I, and T406K;
c) P98A, S102R, K128A, Q153P, G176T, Q203E, G236K, K260N, S283G, S325G, D379E, S382A, A388T, V391I, V394I, and T406K;
d) D54A, P98A, S102R, K128A, Q153P, F163S, D167N, K174E, G176T, A199R, Q203E, D227K, K231A, A234G, G236K, A245S, K260N, D261N, S283G, S291D, S325G, D379A, S382A, A388T, V391I, V394I, D403A, K404E, and T406K;
e) D54A, P98A, S102R, K128A, Q153P, D167N, K174E, G176T, D227K, A234G, K260N, D261N, S283G, S325G, D379A, S382A, A388T, V390I, V391I, V394I, and T406K;
f) D54A, P98A, S102Y, K128A, Q153P, A234G, N235K, G236K, K260N, S283G, S325G, D379E, S382A, A388T, V390I, V391I, V394I, and T406K;
g) D54A, P98A, S102R, K128A, Q153P, Q203E, A234G, N235K, G236N, K260N, S283G, S325G, D379E, S382A, A388T, V391I, V394I, and T406K;
h) P98A, S102R, K128A, Q153P, F163S, D167N, K174E, G176T, A199E, Q203K, D227K, K231A, G236K, A245S, K260N, S283G, S291D, S325G, D379A, S382A, A388T, V391I, V394I, D403A, and T406K;
i) P98A, S102R, K128A, Q153P, F163S, D167N, K174E, G176T, A199R, Q203E, D227K, K231A, G236K, A245S, K260N, S283G, S291D, S325G, D379A, S382A, A388T, V390M, V391I, V394I, D403A, and T406K;
j) D379E, V391I, V394I, and T406K;
k) P98A, S102Y, K128A, Q153P, G236K, K260N, D261N, S283G, S325G, D379E, S382A, A388T, V391I, V394I, and T406K;
l) D54A, P98A, S102R, K128A, Q153P, D167N, K174E, G176T, Q203E, D227K, N235K, G236K, K260N, S283G, S325G, D379A, S382A, A388T, V391I, V394I, and T406K;
m) P98A, S102Y, K128A, Q153P, A234G, G236K, K260N, S283G, S325G, D379E, S382A, A388T, V390I, V391I, V394I, and T406K;
n) D54A, P98A, S102Y, K128A, Q153P, F163S, D167N, K174E, G176T, A199E, Q203K, D227K, K231A, G236K, A245S, K260N, S283G, S291D, S325G, G327N, D379A, S382A, A388T, V391I, V394I, D403A, and T406K; or
o) D54A, P98A, S102Y, K128A, Q153P, A234G, G236K, K260N, S283G, S325G, D379E, S382A, A388T, V391I, V394I, and T406K.

In an embodiment, the present invention provides a nucleic acid sequence encoding the above-described transglutaminase variant of SEQ ID NO: 12.

In another embodiment of the present invention, an expression vector comprises the nucleic acid sequence encoding the transglutaminase variant of SEQ ID NO: 12.

In an embodiment, the present invention provides a host cell transformed with the expression vector comprising the nucleic acid sequence encoding the transglutaminase variant of SEQ ID NO: 12.

In a further embodiment, the present invention provides a method for producing a transglutaminase variant, said method comprising expressing the transglutaminase variant in the transformed host cell, wherein the host cell is transformed with the expression vector comprising the nucleic acid sequence encoding the transglutaminase variant of SEQ ID NO: 12.

The present invention provides a method of processing protein-based food to bond proteins, said method comprising treating the protein-based food with the transglutaminase variant of SEQ ID NO: 12, thereby bonding the protein.

In an embodiment of the method processing protein-based food to bond proteins, the protein-based food is a meat, a fish, imitation crabmeat, processes meat or processed fish.

In another embodiment of the method of processing protein-based food to bond proteins, the treating improves the texture of the protein-based food compared to protein-based food which has not been treated with the transglutaminase variant.

### Xaa-Pro Dipeptidase Variants

Xaa-Pro dipeptidase is an enzyme that catalyzes hydrolysis of dipeptides with a prolyl or hydroxyprolyl residue in the C-terminal position and a nonpolar amino acid at the N-terminal position, and also acts on acts on aminoacyl-hydroxyproline analogs. Xaa-Pro dipeptidase also catalyzes the hydrolysis of toxic organophosphorus cholinesterase-inhibiting compounds, including insecticide paraoxon and nerve gases, such as diisopropylfluorophosphate (DFP), O-isopropyl methylphosphonofluoridate (sarin), O-pinacolyl methylphosphonofluoridate (soman), and O-cyclohexyl methylphosphonofluoridate, thereby making Xaa-Pro dipeptidase a decontaminating agent of subjects contaminated with chemical warfare agents, such as the above-listed organophosphorus nerve agents, insecticide and nerve gases. Xaa-Pro dipeptidase is Mn+2 activated.

The Xaa-Pro dipeptide variants provided herein are stable under conditions such as high temperature, e.g., over 50° C., pH extremes, e.g., high acidity at pH of 4 or lower, in the presence of salts, alkalis and/or surfactants, and other such severe conditions under which wild-type Xaa-Pro dipeptide will denature, decompose and/or under which the Xaa-Pro dipeptide enzymatic function is significantly reduced or eliminated.

The present invention provides a Xaa-Pro dipeptidase variant of a wild-type dipeptidase from Alteromonas sp. Of SEQ ID NO: 13 comprising mutations:
a) I12L, T19Y, E24K, F33I, A72V, T75G, A101T, D102R, F152W, Q162D, K173A, Q197A, N210M, V233Q, A247G, T274A, K276H, I280Q, G293V, T306L, D309E, I312L, A355T, A374P, G392A, D393K, A395R, D398E, N400A, and K412R;
b) V6H, I12L, T19Y, E24K, D29S, F33I, A72V, T75G, A101T, D102E, A138W, E142Q, M144V, F152W, Q162D, K173A, Q197A, N210M, V233Q, A247G, T263H, T274A, K276H, H278M, I280Q, N284A, L291W, G293V, C299M, V303I, T306L, D309E, I312L, D318E, Q353R, A355T, G392A, D393Q, A395R, T397G, D398E, N400A, K412R, and I424C;
c) V6H, I12L, T19Y, E24K, D29S, F33I, A72V, T75G, A101T, D102K, F152W, Q162D, K173A, Q197A, N210M, V233Q, A247G, T274A, K276H, I280Q, L291W, G293V, V303I, T306L, D309E, I312F, D318E, Q353R, A355T, G392A, D393Q, A395K, T397G, D398E, N400A, K412R, and I424C;
d) N2D, K3Q, V6H, E10Q, I12L, K17R, T19Y, E24K, D29A, V31Y, F33I, Q55H, I63V, A72V, T75G, E98D, A101T, D102E, A138W, E142Q, L143H, M144I, F152W, Q162D, K173A, Q177K, G178A, D184E, S191Y, Q197A, L201R, Q204G, S206H, N210T, I224V, F230Y, V233Q, T237E, I243F, A247G, F249Y, T263H, E265Q, V272I, A273N, T274A, K276H, H278M, I280Q, N284A, L286M, G293V, L297E, D298Q, C299M, V303I, T306L, D309E, N311G, I312L, D318E, I320Q, F348W, M349Q, Q353R, A355T, I372V, A374P, Q376M, S389M, G392A, D393E, A395R, T397Q, D398E, N399H, N400A, H402M, K412R, F414Y, and I424C;
e) N2D, V6H, I12L, K17Q, T19Y, E24K, D29S, V31Y, F33I, A72V, T75G, A101T, D102E, A124S, A138W, E142Q, L143Q, M144I, F152W, Q162D, K173A, D184K, S191Y, Q197A N210T, N221S, F230Y, V233Q, A236P, T237E, A247G, F249Y, T263H, E265H, A273N, T274A, K276H, H278M, I280Q, N284A, L286M, L291W, G293V, L297E, D298E, C299A, V303I, T306L, D309E, N311G, I312L, D318E, I320Q, M349Q, Q353R, A355T, K371I, I372V, Q376M, S389M, G392A, D393Q, A395R, T397S, D398E, N400A, K412R, F414Y, and I424C;
f) N2D, K3Q, V6H, E10Q, I12L, K17R, T19Y, E24K, D29S, V31Y, F33I, Q55H, I63V, A72V, T75G, E98D, A101T, D102K, A138W, E142Q, L143H, M144I, F152W, Q162D, K173A, Q177K, G178A, D184E, S191Y, Q197A, L201R, Q204G, S206H, N210T, I224V, F230Y, V233Q, T237E, A247G, F249Y, T263H, E265H, V272I, A273N, T274A, K276H, H278M, I280Q, N284A, L286M, L291W, G293V, L297E, D298Q, C299A, V303I, T306L, D309E, N311G, I312L, I320Q, F348W, M349Q, Q353R, A355T, K371I, I372V, A374P, Q376M, S389M, G392A, D393Q, A395R, T397S, D398E, N399H, N400A, K412R, F414Y, and I424C;
g) V6H, I12L, T19Y, E24K, D29S, F33I, A72V, T75G, A101T, D102K, A138W, E142Q, F152W, Q162D, K173A, Q197A, N210M, V233Q, A247G, T263H, T274A, K276H, H278M, I280Q, N284A, L291W, G293V, C299M, V303I, T306L, D309E, I312L, Q353R, A355T, G392A, D393R, A395R, T397G, D398E, N400A, K412R, and I424C;
h) V6H, I12L, T19Y, E24K, D29A, V31Y, F33I, A72V, T75G, A101T, D102E, A138W, E142Q, M144I, F152W, Q162D, K173A, S191Y, Q197A, N210M, V233Q, T237E, A247G, T263H, E265H, A273N, T274A, K276H, H278M, I280Q, N284A, L291W, G293V, C299M, V303I, T306L, D309E, I312L, D318E, Q353R, A355T, G392A, D393Q, A395R, T397G, D398E, N400A, K412R, and I424C;
i) I12L, T19Y, E24K, F33I, A72V, T75G, A101T, D102S, F152W, Q162D, K173A, Q197A, N210M, V233Q, A247G, T274A, K276H, I280Q, G293V, T306L, D309E, I312L, A355T, K371I, G392A, D393R, A395R, D398E, N400A, and K412R;
j) N2D, V6H, I12L, K17Q, T19Y, E24K, D29S, F33I, A72V, T75G, A101T, D102K, A124S, A138W, E142Q, L143Q, M144R, F152W, Q162D, K173A, D184K, S191Y, Q197A, N210T, N221S, F230Y, V233Q, T237E, A247G, F249Y, T263H, E265H, A273N, T274A, K276H, H278M, I280Q, N284A, L286M, L291W, G293V, L297E, D298E, C299A, V303I, T306L, D309E, N311G, I312L, D318E, I320Q, M349Q, Q353R, A355T, I372V, Q376M, S389M, G392A, D393Q, A395R, T397S, D398E, N400A, H402M, K412R, F414Y, and I424C;
k) V6H, I12L, T19Y, E24K, D29S, F33I, A72V, T75G, A101T, D102K, M144V, F152W, Q162D, K173A, Q197A, N210M, V233Q, A247G, T274A, K276H, I280Q, L291W, G293V, V303I, T306L, D309E, I312L, D318E, Q353R, A355T, K371I, A374P, G392A, D393R, A395R, T397G, D398E, N400A, K412R, and I424C;
l) V6H, I12L, T19Y, E24K, D29S, V31Y, F33I, A72V, T75G, A101T, D102K, A138W, E142Q, F152W, Q162D, K173A, S191Y, Q197A, N210M, V233Q, T237E, A247G, T263H, E265H, A273N, T274A, K276H, H278M, I280Q, N284A, G293V, C299A, V303I, T306L, D309E, I312F, Q353R, A355T, G392A, D393Q, A395R, T397G, D398E, N400A, K412R, and I424C;
m) N2D, V6H, I12L, T19Y, E24K, D29S, V31Y, F33I, A72V, T75G, A101T, D102R, A124S, A138W, E142Q, L143Q, F152W, Q162D, K173A, S191Y, Q197A, N210T, N221S, F230Y, V233Q, T237E, A247G, T263H, E265H, A273N, T274A, K276H, H278M, I280Q, N284A, L291W, G293V, C299M, V303I, T306L, D309E, I312L, Q353R, A355T, G392E, D393Q, A395R, T397G, D398E, N400A, H402M, K412R, F414Y, and I424C ; or
n) N2D, V6A, I12L, T19Y, R20D, E24K, D29S, F33I, A72V, T75G, A101T, D102K, A124S, A138W, E142Q, L143Q, M144R, F152W, Q162D, K173A, S191Y, Q197A, N210T, N221S, F230Y, V233Q, T237E, A247G, T263H, E265H, A273N, T274A, K276H, H278M, I280Q, N284A, G293V, C299M, V303I, T306L, D309E, I312L, D318E, Q353R, A355T, G392A, D393Q, A395R, T397G, D398E, N400A, H402M, K412R, F414Y, and I424C.

In an embodiment, the present invention provides a nucleic acid sequence encoding the above-described Xaa-Pro dipeptidase variant of SEQ ID NO: 13.

In another embodiment of the present invention, an expression vector comprises the nucleic acid sequence encoding the Xaa-Pro dipeptidase variant of SEQ ID NO: 13.

In an embodiment, the present invention provides a host cell transformed with the expression vector comprising the nucleic acid sequence encoding the Xaa-Pro dipeptidase variant of SEQ ID NO: 13.

In a further embodiment, the present invention provides a method for producing a Xaa-Pro dipeptidase variant, said method comprising expressing the Xaa-Pro dipeptidase variant in the transformed host cell, wherein the host cell is transformed with the expression vector comprising the nucleic acid sequence encoding the Xaa-Pro dipeptidase variant of SEQ ID NO: 13.

The present invention provides a method of treating a skin lesion in a subject having an Xaa-Pro dipeptide deficiency comprising administering to the subject a pharmaceutical composition comprising the Xaa-Pro dipeptide variant of SEQ ID NO: 13.

The present invention also provides a method of decontaminating a subject contaminated with a chemical warfare agent comprising administering to the subject a pharmaceutical composition comprising the Xaa-Pro dipeptide variant of SEQ ID NO: 13.

In an embodiment of the method of decontaminating a subject contaminated with a chemical warfare agent, the chemical warfare agent is an organophosphorus nerve agent.

### Xylanase Variants

Xylanases are a class of enzymes that breakdown hemicellulose by cleaving the linear polysaccharide beta-1, 4-xylan into xylose. Fungi, bacteria, yeast, marine algae, protozoans, snails, crustaceans, insect, seeds, produce xylanases, but mammals do not produce this class of enzyme.

Xylanase is a digestive enzyme that improves digestion of hemicellulose-containing food or food additives, such as plant-based food, e.g., cereals/grains, beans and fibrous vegetables, fruits, and nuts.

Xylanase is added to dough as a conditioner to make the dough easier to handle and to improve quality of baked goods, such as crumb structure of bread. Xylanase also is used as a food additive to poultry, in extraction of coffee, plant oils, and starch; in the improvement of nutritional properties of agricultural silage (fermented fodder) and grain feed; and as a biofuel feedstock for anaerobic microbial digestion. Xylanase, in combination with pectinase and cellulose, is used to clarify fruit juices and degum of plant fiber sources, e.g., flax, hemp, jute, and ramie. Xylanase is used to bleach wood pulp in the pulp and papermaking industry.

The xylanase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type xylanase will denature, decompose and/or under which the xylanase enzymatic function is significantly reduced or eliminated.

The present invention provides a xylanase variant of a wild-type xylanase from Aspergillus niger of SEQ ID NO: 14 comprising mutations:
a) L43F, T51H, T58Y, L69I, S88D, L95V, S245P, T256V, E276T, T306N, and T321W;
b) L43F, T51H, S56T, T58Y, L69I, S88D, S92A, L95V, Q143G, T211Q, G243N, S245P, G253S, T256V, V261I, S271A, E276A, E279K, I288V, T306N, and T321W;
c) L43F, T51H, T58Y, L69I, S88D, L95V, Q143G, G243N, S245P, T256V, V261I, S271A, E276A, I288V, T306N, and T321W;
d) L43F, T51H, T58Y, L69I, S88D, L95V, G243N, S245P, T256V, E276T, T306N, and T321W;
e) L43F, T51H, S56T, T58Y, A64R, L69I, S88N, S90T, S92A, L95V, Q122K, Q143G, S206N, L210V, T211Q, G243N, S245P, A250R, G253S, T256V, V261I, S271A, E276A, E279K, I288V, T306N, N315Q, and T321W;
f) L43F, T51H, S56T, T58Y, A64R, L69I, S88N, S90T, S92A, L95V, Q122K, Q143G, S204N, L210V, T211Q, G243N, S245P, A250R, G253A, T256V, V261I, S271A, E276A, E279K, I288V, T306N, N315Q, and T321W;
g) L43F, T51H, T58Y, L69I, S88D, L95V, G243N, S245P, T256V, E276A, I288V, T306N, and T321W; or
h) L43F, T51H, T58Y, L69I, S88D, L95V, G243N, S245P, T256V, E276A, T306N, and T321W.

The present invention also provides a xylanase variant of a wild-type xylanase from Bacillus amyloliquefaciens of SEQ ID NO: 15 comprising mutations:
a) D32H, T43S, A46W, N57T, T71S, F76N, A83K, W86Y, A87N, N91G, T95C, A102N, T121S, N150K, S168G, K182N, L198M, and K204Q;
b) L198M, and K204Q;
c) D32N, T43N, A46W, N57T, T61N, F76N, A83K, V85T, W86Y, A87N, N91G, T95C, A102N, T121S, N150K, S168G, K182Q, L198M, and K204Q;
d) D32H, T43S, A46Y, F76N, A87N, N91G, A102N, S168G, K182N, L198M, and K204Q;
e) D32H, T43S, A46W, F76N, A83K, A87N, N91G, A102N, S168G, K182N, L198M, and K204Q;
f) D32H, T43N, A46W, N57T, F76N, A83K, A87N, N91G, A102N, S168G, K182N, L198M, and K204Q;
g) D32H, T43S, A46W, N57T, F76N, A83K, A87N, N91G, A102N, S168G, K182N, L198M, and K204Q;
h) D32H, T43N, A46Y, N57T, F76N, A83K, W86Y, A87N, N91G, T95C, A102N, S112N, T121S, N150K, S168G, K182N, L198M, and K204Q;
i) D32N, T43S, A46W, N57T, T61N, F76N, A83K, V85T, W86Y, A87N, N91G, T95C, A102N, T121S, N150K, S168G, A171T, K182N, L198M, and K204Q;
j) D32H, T43N, A46W, F76N, A83K, A87N, N91G, A102N, S168G, K182N, L198M, and K204Q;
k) T43S, A46W, F76N, A83K, A87N, N91G, A102N, S168G, K182N, L198M, and K204Q;
l) D32H, T43N, A46W, N57T, F76N, A87N, N91G, A102N, S112N, T121S, S168G, K182N, L198M, and K204Q; or
m) D32H, T43N, A46Y, N57T, F76N, A83K, A87N, N91G, A102N, T121S, S168G, K182Q, L198M, and K204Q.

The present invention further provides a xylanase variant of a wild-type xylanase from Penicillium of SEQ ID NO: 16 comprising mutations:
a) N49S, G58K, T105Q, D136R, K147G, Q148R, D203S, T210S, V229T, A248Q, S249T, E261S, T274A, V279A, D280N, L286C, V292C, A308S, D309S, E310S, S311T, A317S, S318N, and E323P
b) F39L, N49S, G58K, T105Q, D136R, K147G, Q148R, I162A, D203S, T210A, V229T, A248Q, S249T, E261S, A273G, T274S, V279A, D280N, L286C, Q288V, V292C, A308S, D309S, E310S, S311T, A317S, S318N, and E323P
c) F39L, N49S, G58K, T105Q, L115T, D136R, K147G, Q148R, I162A, T210A, V229T, A248Q, S249T, E261S, E267M, A273G, T274S, V279A, D280N, L286C, Q288V, V292C, A308S, D309S, E310S, S311T, A317S, S318N, and E323P;
d) N49S, G58K, T105Q, D136R, K147G, Q148R, D203S, V229T, A248Q, E261S, T274N, V279A, D280N, L286C, V292C, A308S, D309S, E310S, S311T, A317S, S318N, and E323P;
e) K38L, F39L, N49S, G58K, N69D, F99Y, F102W, T105Q, G107N, I110V, L115T, D136R, K147G, Q148R, L153C, I162A, F182Y, V183I, R184A, I201V, T210A, V229M, A244G, A247D, A248Q, S249T, G250E, A251K, T260A, E261S, V265I, A273G, T274S, V279A, D280N, L286C, Q288V, V292C, A308S, D309S, E310S, S311T, A317S, S318N, and E323P;
f) N49T, G58K, T105Q, L115T, D136R, K147G, Q148R, V229T, A248Q, T274N, D280N, L286C, V292C, A308S, D309S, E310S, S311T, A317S, S318N, and E323P
g) N49S, G58K, T105Q, L115T, D136R, K147G, Q148R, T210S, V229T, A248Q, S249T, E261S, T274N, V279A, D280N, L286C, V292C, A308S, D309S, E310S, S311T, A317S, S318N, and E323P;
h) N49T, G58K, T105Q, D136R, K147S, Q148R, D203S, V229T, A248Q, T274N, D280N, L286C, V292C, A308S, D309S, E310S, S311T, A317S, S318N, and E323P;
i) N49T, G58K, T105Q, L115T, D136R, K147G, Q148R, V229T, A248Q, L286C, V292C, A308S, D309S, E310S, S311T, A317S, S318N, and E323P;
j) K38L, F39L, N49S, G58K, N69D, F99Y, F102W, T105Q, G107N, I110V, D136R, K147G, Q148R, L153C, I162A, F182Y, V183I, R184A, I201V, D203S, T210A, V229T, A244G, A247D, A248Q, S249T, G250E, A251K, T260A, E261S, V265I, A273G, T274S, V279A, D280N, L286C, Q288V, V292C, A308S, D309S, E310S, S311T, A317S, S318N, and E323P;
k) K38L, F39L, N49S, G58K, N69D, F102W, T105Q, G107N, D136R, K147G, Q148R, I162A, F182Y, V183I, R184A, D203S, T210A, V229T, A248Q, S249T, E261S, A273G, T274S, V279A, D280N, L286C, Q288V, V292C, A308S, D309S, E310S, S311T, A317S, S318N, and E323P; or
l) L286C, V292C, A308S, D309S, E310S, S311T, A317S, S318N, and E323P.

The present invention still further provides a xylanase variant of a wild-type xylanase from Talaromyces pinophilus of SEQ ID NO: 29 comprising mutations:
a) T68G, E79A, T84N, P101S, L149V, E166Q, S173K, Q184N, E189S, K202A, N203S, M206L, N207H, T210Q, L211M, F213Y, I216V, A218V, Y229Q, A230T, and N232S;
b) T68G, S73H, T76N, E79S, T84N, P101S, L149V, N161V, E166Q, S173R, Q184R, E189S, T193N, Q195A, K202A, N203S, M206L, N207H, L211M, F213Y, I216V, A218V, Y229I, A230T, and N232S;
c) T68G, S73H, E79S, T84N, P101S, L149V, N161V, E166Q, S173R, Q184R, E189S, T193N, Q195A, H197F, K202A, N203S, M206L, N207H, L211M, F213Y, I216V, A218V, Y229I, A230T, and N232S;
d) T68G, P101S, E166Q, Q184N, E189S, K202A, N203S, M206L, N207H, T210Q, L211M, F213Y, I216V, A218V, Y229Q, A230S, and N232S;
e) T68G, E79A, P101S, E166Q, S173R, Q184N, E189S, K202A, N203S, M206L, N207H, T210Q, L211M, F213Y, I216V, A218V, Y229Q, A230S, and N232S;
f) T68G, E79T, T84N, P101S, L149V, E166Q, S173K, Q184N, E189S, K202A, N203S, M206L, N207H, T210Q, L211M, F213Y, I216V, A218V, Y229Q, A230T, and N232S;
g) Q184N, E189S, K202A, N203S, M206L, N207H, T210Q, L211M, F213Y, I216V, A218V, Y229Q, A230S, and N232S
h) T68G, S73H, T76N, E79S, T84N, P101S, L149V, N161V, E166Q, S173R, Q184R, E189S, T193N, Q195A, H197F, K202A, N203S, M206L, N207H, L211M, F213Y, I216V, A218V, Y229I, A230T, and N232S;
i) T68G, S73H, E79S, T84N, P101S, L149V, N161V, E166Q, S173R, Q184R, E189S, K202A, N203S, M206L, N207H, T210Q, L211M, F213Y, I216V, A218V, Y229Q, A230T, and N232S; or
j) T68G, S73H, E79S, T84N, P101S, L149V, N161C, E166Q, S173R, Q184R, E189S, T193N, Q195A, K202A, N203S, M206L, N207H, L211M, F213Y, I216V, A218V, Y229I, A230T, and N232S.

The present invention provides a xylanase variant of a wild-type xylanase from Thermomyces lanuginosus of SEQ ID NO: 30 comprising mutations:
a) R147D, A156Q, and Q182T;
b) I145T, R147D, A156Q, Q182T, A191R, and N198G;
c) S69T, A88N, D128N, I145T, R147D, A156Q, Q182T, A191R, and N198G;
d) I145T, R147D, A156Q, Q182T, and N198G;
e) S69T, A88N, F124Y, D128N, D135K, I145T, R147D, A156Q, D160E, Q182T, A191R, L195M, and N198G;
f) S69T, A88N, D128N, I145T, R147D, A156Q, Q182N, A191R, N198G;
g) R147D, A156Q, Q182T, and N198G;
h) S69E, A88N, F124Y, D128N, D135K, I145T, R147D, A156Q, D160E, Q182N, A191R, L195M, and N198G;
i) I145T, R147D, A156Q, Q182N, and N198G; or
j) R147D, A156Q, Q182N, and N198G.

The present invention also provides a xylanase variant of a wild-type xylanase from Bacillus paralicheniformis of SEQ ID NO: 31 comprising mutations:
a) A33V, K99Q, S100R, I102A, K103S, I107M, S130N, A140Q, W177C, M187I, K188A, L207N, I210M, L227V, D252E, S284C, D289N, K306W, Y316A, K348S, K360N, G364Q, N368T, S375T, L390M, G393T, N398T, T400S, D401G, H407W, and A418V;
b) K99Q, S100R, I102A, I107M, A140Q, T151E, L207N, L227V, D252R, S284C, Y316A, K348S, K360N, L390M, N398T, T400S, D401G, H407W, and A418V;
c) A33V, K99Q, S100R, I102A, K103S, I107M, S130N, A140Q, W177C, M187I, K188A, L207N, I210M, L227V, D252E, S284C, D289N, K348S, K360N, G364Q, N368T, S375T, L390M, G393T, N398T, T400S, D401G, H407W, and A418V;
d) A33V, E65Q, Y92N, K99Q, S100R, K103A, I107K, V108L, N124T, K144A, K188A, I199M, L207N, K219T, L227V, D252R, H280E, S284C, K306R, T310N, K348S, D350G, L390M, G393T, N398T, T400S, D401G, H407W, and A418V;
e) A33V, R36A, R64M, E65Q, E86P, N90D, K99Q, S100R, K103A, H104R, I107K, V108L, V102I, N124T, S130D, K144A, F152Y, L184A, M187I, K188A, A196C, I199M, L207N, I210M, K219T, L227V, T234G, N237S, G248H, D252R, S278A, H280E, S284C, D289N, K306R, T310N, K348S, D350G, V353I, N368T, L390M, G393T, N396D, N398T, T400S, D401G, H403Y, H407W, and A418V;
f) A33V, E65Q, Y92N, K99Q, S100R, K103A, I107K, V108L, N124T, K144A, K188A, I199M, L207N, K219T, L227V, D252R, H280E, S284C, K306R, T310N, K348S, D350G, N368Q, L390M, G393T, N398T, T400S, D401G, H407W, and A418V;
g) H83G, K99Q, S100R, I102Q, K103A, A140Q, W177C, L207N, L227V, D252E, S284C, D289N, K348S, K360N, S375T, L390M, G393T, N398T, T400S, D401G, H407W, and A418V;
h) K99Q, S100R, I102A, K103A, A140Q, K154G, W177C, L207N, L227V, D252N, S284C, D289N, Y316A, K348S, K360N, S375T, L390M, G393T, N398T, T400S, D401G, H407W, and A418V;
i) K99Q, S100R, I102A, K103A, I107M, A140Q, W177C, L207N, L227V, D252N, S284C, Y316A, K348T, K360N, S377T, L390M, G393T, N398T, T400S, D401G, H407W, and A418V;
j) A33V, R36H, A39T, K41H, V43T, H83G, N90E, K99N, S100R, I102Q, K103A, H104M, I107T, S117A, N124S, S130N, A140Q, K144Q, F152Y, K154Q, W177C, I183F, L184R, M187L, K188A, A196C, L207N, I210M, P218A, K219T, L227V, A229T, T234G, N237S, G248H, D252R, Y260H, S278A, S284C, D289N, T310N, N317S, H320Q, Y343C, K348S, G349S, D350G, K360N, G364A, N368T, S375T, S380T, I383M, L390M, Q391A, G393T, N396D, N398T, T400S, D401G, H403R, H407W, and A418I;
k) A33V, E65Q, H83G, N90D, K99Q, S100R, K103A, I107K, V108L, N124T, K144A, L184A, K188A, I199M, L207N, I210M, K219T, L227V, T234G, D252R, H280E, S284C, K306R, T310N, K348S, D350G, L390M, G393T, N398T, T400S, D401G, H407W, and A418V;
l) A33V, E65Q, S100R, K103E, N124T, K144A, K188A, I199M, L207N, L227S, D252R, S284C, K306R, T310N, K348S, G393T, N398T, T400S, D401G, H407W, and A418V;
m) S100R, K103E, N124T, K144A, L207N, L227S, D252R, K306R, K348S, N398T, T400S, D401G, H407W, and A418V;
n) A33V, R36S, A39T, K41H, V43T, N90E, K99Q, S100R, I102Q, K103A, H104L, I107T, S117A, N124S, S130D, A140Q, K144Q, F152Y, K154Q, W177C, I183F, L184R, M187L, K188A, N195S, A196C, L207N, I210M, P218A, K219T, L227V, T234G, N237S, G248H, D252R, Y260H, S278A, S284C, D289N, T310N, Y316A, K348S, G349S, D350G, K360N, G364Q, N366T, N368T, S375T, S377T, S380T, I383M, L390M, G393T, N396D, N398T, T400S, D401G, H403R, H407W, and A418I;
o) A33V, V43T, K99Q, S100R, I102Q, K103A, H104R, I107T, N124S, S130N, A140Q, K144A, D148S, F152Y, W177C, L184R, M187I, K188A, A196C, L207N, I210M, K219T, L227V, T234G, Q235S, G248H, D252N, S284C, D289N, T310N, Y316A, K348S, D350G, K360N, G364A, S375T, S377T, L390M, G393T, N398T, T400S, D401G, H403R, H407N, and A418I;
p) K99Q, S100R, I102A, A140Q, T151E, W177C, L207N, L227V, D252R, S284C, K348S, K360N, L390M, N398T, T400S, D401G, H407W, and A418V;
q) S100R, K103E, N124T, K144A, I199L, L207N, L227V, D252R, K306R, K348S, N368Q, N398T, T400S, D401G, H407W, and A418V;
r) K99Q, S100R, I102A, K103A, L207N, L227V, D252E, S284C, K348T, K360N, L390M, G393T, N398T, T400S, D401G, H407W, and A418V;
s) A33V, R64M, E65Q, N90D, K99Q, S100R, K103A, H104N, I107K, V108L, V120I, N124T, K144A, F152Y, L184A, K188A, A196C, I199M, L207N, I210M, K219T, L227V, T234G, G248H, D252R, S278A, H280E, S284C, K306R, T310N, K348S, D350G, L390M, G393T, N398T, T400S, D401G, H407W, and A418V;
t) A33V, V43T, A61P, F68I, H83G, K99Q, S100R, I102Q, K103A, H104N, I107T, N124S, S130N, F152Y, M187I, K188A, A196C, L207N, I210M, K219T, L227V, T234G, G248H, D252N, S284C, D289N, Y316A, K348S, K360N, G364T, S375T, S377T, L390M, G393T, N398T, T400S, D401G, H407Y, and A418I;
u) N398T, T400S, D401G, and A418V;
v) A33V, V43T, H83G, K99Q, S100R, I102Q, K103A, H104N, I107T, N124S, S130N, A140Q, F152Y, W177C, M187I, K188A, A196C, L207N, I210M, K219T, L227V, T234G, Q235S, G248H, D252N, S284C, D289N, Y316A, K348S, K360N, G364Q, N368T, S375T, S377T, L390M, G393T, N398T, T400S, D401G, H407Y, and A418I;
w) A33V, E65Q, S100R, K103E, N124T, K144A, K188A, I199M, L207N, L227V, D252R, S284C, K306R, T310N, K348S, G393T, N398T, T400S, D401G, H407W, and A418V;
x) A33V, R64M, E65Q, H83G, N90D, K99Q, S100R, K103A, H104N, I107K, V120I, N124T, K144A, F152Y, L184A, K188A, A196C, I199M, L207N, I210M, K219T, L227V, T234G, G248H, D252R, S278A, H280E, S284C, K306R, T310N, K348S, D350G, L390M, G393T, N398T, T400S, D401G, H407W, and A418V;
y) A33V, V43T, K99Q, S100R, I102Q, K103A, H104R, I107T, N124S, S130N, A140Q, K144A, D148S, F152Y, W177C, L184R, M187I, K188A, A196C, L207N, I210M, K219T, L227V, T234G, G248H, D252N, S284C, D289N, T310N, Y316A, K348S, D350G, K360N, G364A, S375T, S377T, L390M, G393T, N398T, T400S, D401G, H403R, H407W, and A418I;
z) S100R, K103E, N124T, K144A, I199L, L207N, L227V, D252R, K306R, K348S, N398T, T400S, D401G, H407W, and A418V; or
aa) A33V, R36A, R64M, E65Q, E86P, N90D, K99Q, S100R, K103A, H104R, I107K, V108L, V102I, N124T, S130D, K144A, H145W, F152Y, L184A, M187I, K188A, A196C, I199M, L207N, I210M, K219T, L227V, T234G, N237S, G248H, D252R, S278A, H280E, S284C, D289N, K306R, T310N, K348S, D350G, V353I, N368T, L390M, G393T, N396D, N398T, T400S, D401G, H403Y, H407W, and A418V.

The present invention also provides a xylanase variant of a wild-type xylanase from Actinomadura sp of SEQ ID NO: 32 comprising mutations:
a) F55Y, S62G, S79T, T83N, A88C, G96S, R97G, V100I, A104G, T115A, V130I, S132N, W136Y, T145S, I146V, Q157Y, S191A, R200K, Y201H, S207T, M211Q, Y223S, and V228L;
b) G96S, R97G, A104G, W136Y, T145S, I146V, Q157Y, S191A, Y201H, S207T, Y223S, and V228L;
c) R97G, T145S, Q157Y, S191A, Y201H, S207T, Y223S, and V228L;
d) F55Y, S62G, S79T, T83N, A88C, R97G, V100I, A104G, T115A, V130I, S132N, W136Y, T145S, I146V, Q157Y, S191A, R200K, Y201L, S207T, M211Q, Y223S, and V228L;
e) R97G, W136Y, T145S, Q157Y, S191A, Y201H, S207T, Y223S, and V228L;
f) F55Y, G96S, R97G, A104G, T115A, W136Y, T145S, I146V, Q157Y, S191A, Y201H, S207T, Y223S, and V228L; or
g) S191A, Y201H, N204H, S207T, Y223S, and V228L.

The present invention further provides a xylanase variant of a wild-type xylanase from Aspergillus niger of SEQ ID NO: 33 comprising mutations:
a) V29T, D32H, F35A, K36A, A37R, T45S, N83S, S101K, G103N, L111T, V112L, G128T, Q132R, G212A, N215A, H216K, V221L, A238S, A240Q, N249Q, G253S, A269G, K279Q, L282C, V288C, S306E, S307T, A319P, T322N, I324C, and A325V;
b) V29T, K36A, A37Q, T45S, S101K, G103N, G128T, Q143G, V149C, R180E, N215A, H216N, V225I, A240Q, G253S, G255N, K257S, A269G, L282C, V288C, S306E, S307T, A319P, T322N, and I324A;
c) V29T, K34Q, F98L, S101Q, G103N, G128A, Q143G, R180E, V189A, N215A, H216K, A240Q, N249Q, G253S, K257S, A269G, K279E, L282C, V288C, S307T, A319P, T322N, and I324A;
d) V29T, G128A, Q143G, R180E, K257S, A269G, K279E, L282C, V288C, S307T, A319P, T322N, and I324A;
e) G128K, Q143G, K257S, A269G, K279E, L282C, V288C, S307T, A319P, and I324A;
f) V29T, D32H, K36A, A37R, T45S, S101K, G103N, L111T, G128T, Q132R, N215A, H216K, V221L, A240Q, G253S, A269G, K279Q, L282C, V288C, S307T, A319P, T322N, I324C, and A325V;
g) V29T, D32H, F35M, K36A, A37R, T45S, N83S, S101K, G103N, L111T, G128A, Q132R, N215A, H216K, V221L, A240Q, N249Q, G253S, A269G, K279Q, L282C, V288C, V295I, S307T, A319P, T322N, I324C, and A325V;
h) D32N, K36A, L111T, G128T, Q132R, G253S, A269G, K279Q, L282C, V288C, S307T, A319P, and I324C;
i) V29T, K34Q, F98L, S101Q, G103N, G128A, Q143G, R180E, V189A, N215A, H216N, A240Q, N249Q, G253S, K257S, A269G, K279E, L282C, V288C, S307T, A319P, T322N, and I324A;
j) V29T, K34Q, K36Q, F98L, S101Q, G103N, G128A, Q143G, I158A, R180E, V189A, G212A, N215A, H216K, V221I, V225I, A238S, A240Q, N249Q, G253S, K257S, A269G, V277I, L282C, V288C, S307T, A319P, T322N, and I324A;
k) V29T, F98L, S101Q, G103N, G128A, Q143G, R180E, V189A, N215A, H216N, A240Q, G253S, K257S, A269G, K279E, L282C, V288C, S307T, A319P, T322N, and I324A;
l) G128T, Q143G, G255N, K257S, A269G, L282C, V288C, S307T, A319P, and I324A;
m) V29T, K34Q, K36A, A37K, T45S, S101K, G103N, G128A, Q132S, Q143G, V149C, R180E, G212A, N215A, H216K, K218D, V221L, V225I, A238S, A240Q, N249Q, G253S, G255N, T256A, K257S, A269G, V277I, L282C, V288C, S306E, S307T, A319P, T322N, and I324A;
n) V29T, D32H, F35M, K36A, T45S, N83S, S101K, G103N, L111T, G128T, Q132R, N215A, H216K, V221L, A240Q, N249Q, G253S, A269G, K279Q, L282C, V288C, S307T, A319P, T322N, I324C, and A325V;
o) S101T, G128T, Q143G, G253S, G255N, K257S, A269G, L282C, V288C, S307T, A319P, and I324A;
p) V29T, K34Q, K36A, A37Q, T45S, S101K, G103N, G128T, Q143G, V149C, R180E, N215A, H216N, V225I, A240Q, L251F, G253S, G255N, K257S, A269G, L282C, V288C, S306E, S307T, A319P, T322N, and I324A;
q) V29T, K34E, F35M, K36A, A37K, T45S, S101K, G103N, G128K, Q132S, Q143G, V149C, R180E, G212A, N215A, H216K, K218D, V221L, V225I, A238S, A240Q, N249Q, G253S, G255N, T256A, K257S, A269G, V277I, L282C, V288C, S306E, S307T, A319P, T322N, I324A, and A325L;
r) V29A, D32H, K36A, A37R, T45S, S101K, G103N, L111T, G128T, Q132R, N215A, H216K, V221L, A240Q, G253S, A269G, K279Q, L282C, V288C, S307T, A319P, T322N, I324C, and A325V;
s) D32H, K36A, L111T, G128T, Q132R, G253S, A269G, K279Q, L282C, V288C, S307T, and A319P;
t) D32H, K36A, L111T, G128T, G253S, A269G, K279Q, L282C, V288C, S307T, and A319P;
u) G128A, Q143G, K257S, A269G, K279E, L282C, V288C, S307T, A319P, and I324A;
v) V29T, K34E, K36Q, F98L, S101Q, G103N, G128K, Q143G, I158A, R180E, V189A, G212A, N215A, H216N, V221I, V225I, A238S, A240Q, N249Q, G253S, K257S, A269G, V277I, L282C, V288C, S307T, A319P, T322N, and I324A;
w) G128A, Q143G, K257S, A269G, L282C, V288C, S307T, and A319P;
x) V29T, G103N, G128T, Q143G, R180E, V225I, G253S, G255N, K257S, A269G, K279Q, L282C, V288C, S307T, A319P, T322N, and I324A;
y) S101T, G128T, Q143G, G255N, K257S, A269G, L282C, V288C, S307T, A319P, and I324A;
z) V29T, S101T, G103N, G128T, Q143G, R180E, V225I, G253S, G255N, K257S, A269G, K279Q, L282C, V288C, S307T, A319P, T322N, and I324A;
aa) V29T, D32N, K36A, T45S, G103N, L111T, G128T, Q132R, N215A, A240Q, G253S, A269G, K279Q, L282C, V288C, S307T, A319P, T322N, and I324C;
bb) V29T, T45S, S101K, G103N, G128T, Q143G, R180E, V225I, A240Q, G253S, G255N, K257S, A269G, K279E, L282C, V288C, S306E, S307T, A319P, T322N, and I324A;
cc) V29T, D32H, K34Q, F35M, K36A, A37R, T45S, N83S, S101K, G103N, L111T, V112L, G128T, Q132R, G212A, N215A, H216K, V221L, A238S, A240Q, N249Q, G253S, A269G, K279Q, L282C, V288C, S307T, A319P, T322N, I324C, and A325V; or
dd) V29T, F98L, S101Q, G103N, G128K, Q143G, R180E, V189A, N215A, H216N, A240Q, G253S, K257S, A269G, K279E, L282C, V288C, S307T, A319P, T322N, and I324A; or
ee) L282C, V288C, S307T, and A319P.

The present invention provides a xylanase variant of a wild-type xylanase from Trichoderma reesi of SEQ ID NO: 34 comprising mutations:
a) S53G, S67D, D83Q, T92Q, A97R, S104Q, N108E, E163Q, N180Q, H203N, L204M, and S226T;
b) S53G, Q65N, S67C, N78T, D83Q, T92S, A97R, S104Q, N108E, E163Q, N180Q, H203N, L204M, M207H, and S226T;
c) S53G, S67D, A97R, S104Q, N108E, E163Q, H203N, and S226T;
d) S53G, S67D, A97R, S104Q, N108E, E163Q, N180Q, H203N, and S226T;
e) S53G, Q65N, S67D, D83Q, T92S, A97R, S104Q, N108E, E163Q, N180Q, H203N, L204M, and S226T;
f) A97R, and E163Q;
g) S53G, Q65N, S67C, N78T, D83Q, T92Q, A97R, S104Q, N108E, E163Q, N180Q, H203N, L204M, M207H, and S226T;
h) S53G, A97R, and E163Q; or
i) S53G, Q65N, S67C, D83Q, T92S, A97R, S104Q, N108E, E163Q, N180Q, H203N, L204M, and S226T.

In an embodiment, the present invention provides a nucleic acid sequence encoding the above-described xylanase variant of any one of SEQ ID NO: 14 to SEQ ID NO: 16 or SEQ ID NO: 29 to SEQ ID NO: 34.

In another embodiment of the present invention, an expression vector comprises the nucleic acid sequence encoding the xylanase variant of any one of SEQ ID NO: 14 to SEQ ID NO: 16 or SEQ ID NO: 29 to SEQ ID NO: 34.

In an embodiment, the present invention provides a host cell transformed with the expression vector comprising the nucleic acid sequence encoding the xylanase variant of any one of SEQ ID NO: 14 to SEQ ID NO: 16 or SEQ ID NO: 29 to SEQ ID NO: 34.

In a further embodiment, the present invention provides a method for producing a xylanase variant, said method comprising expressing the xylanase variant in the transformed host cell, wherein the host cell is transformed with the expression vector comprising the nucleic acid sequence encoding the xylanase variant of any one of SEQ ID NO: 14 to SEQ ID NO: 16 or SEQ ID NO: 29 to SEQ ID NO: 34.

In an embodiment, this invention provides a method for aiding or supporting digestion of xylan in a subject, said method comprising administering to the subject a pharmaceutical composition comprising the xylanase variant of any one of SEQ ID NO: 14 to SEQ ID NO: 16 or SEQ ID NO: 29 to SEQ ID NO: 34.

In another embodiment, this invention provides a method for bleaching wood pulp, said method comprising treating the wood pulp with the xylanase variant of any one of SEQ ID NO: 14 to SEQ ID NO: 16 or SEQ ID NO: 29 to SEQ ID NO: 34.

In still another embodiment, this invention provides a method for bleaching wood pulp, said method comprising treating the wood pulp with the xylanase variant of any one of SEQ ID NO: 14 to SEQ ID NO: 16 or SEQ ID NO: 29 to SEQ ID NO: 34.

In a further embodiment, this invention provides a method baking a food product, said method comprising adding to a flour-containing dough the xylanase variant of any one of SEQ ID NO: 14 to SEQ ID NO: 16 or SEQ ID NO: 29 to SEQ ID NO: 34, wherein the addition of the xylanase variant improves the baked food product volume and crumb structure thereof compared to a baked food product to which the xylanase variant has not been added.

### Glucanase Variants

Glucanases are enzymes that hydrolyze glucan, a polysaccharide made of glucose subunits. Glucanases include alpha-glucanases, e.g., pullulanase, and beta-glucanases. Beta-glucanases break down glycosidic bonds in beta-glucan. Beta-glucan is a type of indigestible polysaccharide found in cellulose plant fiber, cereal bran fiber, and parts of mushrooms. Beta-glucans are present in oats, wheat, barley, rye, wheat, sorghum, reishi mushrooms, shiitake mushrooms, maitake mushrooms, yeasts, seaweed and algae.

The specific glucanase, beta-glucan, enhances digestion of beta-glucan contained in the above-described plant-based food, cereal grains and mushrooms. Glucanase aids in easing indigestion, bloating abdominal discomfort and gas.

Overgrowth of a fungus, such as Candida, or an overabundance of beta-glucan in a digestive tract of a subject creates biofilms produced by the fungus or bacteria in the digestive tract. The glucanase variants provided herein are used in treating such an overgrowth of a fungus or an overabundance of beta-glucan in the digestive tract of a subject.

Functional foods and beverages, as well as functional animal feed, provide health benefits, via an added function, in addition to the basic nutrition found in the food and beverage; the added function may be from added active components, e.g., probiotics, or inclusion of components that have been modified, e.g., by the addition of vitamins, minerals, antioxidants, omega-3 fatty acids, and/or fiber.

The glucanases provided herein are used to prepare functional food, functional beverages and functional animal feed by pre-treating a beta-glucan containing food, beverage or animal feed with one or more glucanases; this pre-treatment releases nutrients from the beta-glucan containing food, beverage or animal feed that would otherwise remain undigested, increasing the nutritional value of the pre-treated food, beverage or animal feed.

The glucanases provided herein are used to aiding in the color and texture of wine. The glucanases provided herein are used to treat waste, including food waste and farm waste, said types of waste, e.g., containing oats, wheat, barley, rye, wheat, and/or sorghum, to hydrolyze the beta-glucans and reduce the viscosity of the waste.

The glucanase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type glucanase will denature, decompose and/or under which the glucanase enzymatic function is significantly reduced or eliminated.

The present invention provides a glucanase variant of a wild-type glucanase from Bacillus megaterium of SEQ ID NO: 19 comprising mutations:
a) I44V, T47N, K55N, P56K, L67H, Y73C, D77A, K80D, I88A, N110A, L122R, I124M, I128V, N141H, S154A, L155K, S158N, S159T, A167C, D173G, S189D, D195G, T202V, D216A, D217N, K220N, A222S, A227T, G236K, Q237S, F238W, D244S, S248Q, I253V, Q273E, E276R, N279D, Y280W, P315R, T324K, V326Y, N329A, and R331L;
b) I44V, K55N, P56K, Y73C, D77A, K80D, I88A, I103F, N110A, I124M, N134T, N141H, S154N, L155K, S158N, S159T, D173G, S189A, D195G, T202V, D216A, D217N, K220N, A222D, A227T, G236K, Q237S, F238W, D244S, S248Q, I253V, Q273E, E276R, N279D, Y280W, P315R, T324K, and R331L;
c) K80M, I88A, I124M, D173G, S189D, D195G, T202V, A227T, D244S, S248K, Y280W, P315R, T324K, and R331L;
d) I44V, P56A, Y73C, D77A, K80D, I88A, I103F, N110A, K120L, I124M, S154N, L155K, S159T, D173G, S189D, D195G, T202V, D216A, D217N, K220N, A222D, A227T, F238W, D244S, S248K, Q273E, Y280W, P315R, T324K, and R331L;
e) I44V, I62V, Y73C, V74Y, D77A, K80D, L82F, I88A, F91M, K113T, I124M, L155K, S158N, S189D, D195G, T202C, D213S, D216A, D217N, K220N, A227T, F238W, D244R, D262S, Q273E, Y280W, N290S, S300D, P315R, T324K, V326Y, and N329Q;
f) I44V, I62V, V74Y, D77A, K80D, L82F, I88C, F91M, K113T, I124M, L155K, S158N, S189N, D195G, T202C, D216A, D217N, K220N, A227T, F238W, D244R, D262T, Y280W, N290S, P315R, T324K, V326Y, and N329Q;
g) I44V, Y73C, K80D, I88A, I124M, L155K, D173G, D195G, T202V, D216A, D217N, K220N, A222D, A227T, F238W, D244S, S248K, Q273E, Y280W, P315R, T324I, and R331L;
h) I44V, P56A, Y73C, D77A, K80D, I88A, N110A, I124M, S154N, L155K, S159T, D173G, S189D, D195G, T202V, D216A, D217N, K220N, A222D, A227T, F238W, D244S, S248K, Q273E, Y280W, P315R, T324K, and R331L;
i) I44V, K80D, I88A I103F, I124M, N134T, S154N, L155K, D173G, D195G, T202V, K220N, A222D, A227T, D244S, S248Q, Y280W, P315R, T324K, and R331L;
j) I62V, V74Y, K80D, L82F, I88C, F91M, K113T, I124M, S189N, D195G, T202C, K220N, A227T, D244R, D262T, Y280W, P315R, T324K, and V326Y;
k) P315R, and R331L;
l) I44V, I62V, V74Y, D77A, K80D, L82F, I88C, F91M, K113T, I124M, L155K, S158N, D195G, T202C, D216A, D217N, K220N, A227T, F238W, D244R, D262T, Y280W, N290S, P315R, T324K, and V326Y;
m) I44V, T47N, K55N, P56K, Y73C, D77A, K80D, I88A, I103F, N110A, L122R, I124M, I128V, N134T, N141H, S154A, L155K, S158N, S159T, A167C, D173G, S189A, D195G, T202V, D216A, D217N, K220N, A222D, A227T, G236K, Q237S, F238W, D244S, S248Q, I253V, Q273E, E276R, N279D, Y280W, P315R, T324K, V326Y, N329A, and R331L;
n) K80M, I88A, I103F, I124M, D173G, S189D, D195G, T202V, A227T, D244S, S248K, Y280W, P315R, T324K, and R331L;
o) I44V, K55N, P56K, Y73C, D77A, K80D, I88A, I103F, N110A, I124M, N141H, L155K, S158N, S159T, D173G, S189A, D195G, T202V, D216A, D217N, K220N, A222D, A227T, G236K, Q237S, F238W, D244S, S248Q, I253V, Q273E, E276R, N279D, Y280W, P315R, T324K, and R331L;
p) I44V, K45C, K55N, I62M, L67T, Y73C, V74Y, D77A, K80D, L82F, R83A, I88A, F91M, I103M, N110A, K113T, I124M, K150T, L155K, S158N, S159T, S189N, D195G, T202C, D213S, D216A, D217N, K220N, A222D, A227T, F238W, D244R, S248N, D262T, Y270N, Q273E, Y280W, N290G, S300D, P315R, T324K, V326Y, and N329Q;
q) I44V, K80D, I88A, I124M, L155K, D173G, D195G, T202V, K220N, A222D, A227T, D244S, S248K, Y280W, P315R, T324K, and R331L;
r) I44V, Y73C, K80D, I88A, I103F, I124M, S154N, L155K, D173G, D195G, T202V, D216K, K220N, A222D, A227T, F238W, D244S, S248K, Q273E, Y280W, P315R, T324K, and R331L;
s) I44V, K45C, K55N, I62M, L67T, Y73C, V74Y, D77A, K80D, L82F, R83A, I88A, F91M, I103M, N110A, K113T, I124M, K150T, L155K, S158N, S159T, S189D, D195G, T202C, D213K, D216A, D217N, K220N, A222D, A227T, F238W, D244Q, S248N, D262S, Y270N, Q273E, Y280W, N290G, P315R, T324K, V326Y, and N329Q;
t) I62V, V74Y, K80D, L82F, I88A, F91M, K113T, I124M, D195G, T202C, K220N, A227T, D244R, D262T, Y280W, N290S, P315R, T324K, V326Y, and N329Q;
u) I44V, K45C, T47N, K55N, I62M, L67T, Y73C, V74Y, D77A, K80D, L82F, R83A, D84H, G87N, I88A, F91M, I103M, N110A, K113T, K120T, L122R, I124M, N141H, K150T, L155K, S158N, S159T, K177A, K181R, S189D, D195G, T202C, T206D, N212D, D213E, D216A, D217N, K220N, A222S, A227T, Q237D, F238W, D244R, S248N, I253V, G259A, D262S, Y270N, Q273E, E276R, Y280W, N290G, P315R, T324K, V326Y, and N329Q;
v) I44V, I62V, Y73C, V74Y, D77A, K80D, L82F, I88A, F91M, K113T, I124M, L155K, S158N, S189D, D195G, T202C, D213S, D216A, D217N, K220N, A227T, F238W, D244R, D262T, Q273E, Y280W, N290S, S300D, P315R, T324K, V326Y, and N329Q;
w) I62V, V74Y, K80D, L82F, I88A, F91M, K113T, I124M, S189N, D195G, T202C, K220N, A227T, D244R, D262T, Y280W, N290S, P315R, T324K, and V326Y;
x) I44V, I62V, Y73C, V74Y, D77A, K80D, L82F, I88A, F91M, K113T, I124M, L155K, S158N, S189D, D195G, T202C, D213E, D216A, D217N, K220N, A222D, A227T, F238W, D244Q, S248N, D262S, Y270N, Q273E, Y280W, N290S, P315R, T324K, V326Y, and N329Q;
y) I44V, I62V, Y73C, V74Y, D77A, K80D, L82F, I88C, F91M, K113T, I124M, L155K, S158N, K177Q, D195G, T202C, D213S, D216A, D217N, K220N, A222D, A227T, F238W, D244R, S248N, G259A, D262S, Y270N, Q273E, Y280W, N290S, P315R, T324K, V326Y, and N329Q; or
z) I44V, K45C, T47N, K55N, I62M, L67T, Y73C, V74Y, D77A, K80D, L82F, R83A, D84H, G87N, I88A, F91M, I103M, N110A, K113T, K120T, L122R, I124M, N141H, K150T, L155K, S158N, S159T, K177A, K181R, S189D, D195G, T202C, T206D, N212D, D213E, D216A, D217N, K220N, A222S, A227T, Q237D, F238W, D244R, S248N, I253V, D262T, Y270N, Q273E, E276R, Y280W, N290G, S300D, P315R, T324K, V326Y, and N329Q.

In an embodiment, the present invention provides a nucleic acid sequence encoding the above-described glucanase variant of SEQ ID NO: 19.

In another embodiment of the present invention, an expression vector comprises the nucleic acid sequence encoding the glucanase variant of SEQ ID NO: 19.

In an embodiment, the present invention provides a host cell transformed with the expression vector comprising the nucleic acid sequence encoding the glucanase variant of SEQ ID NO: 19.

In a further embodiment, the present invention provides a method for producing a glucanase variant, said method comprising expressing the glucanase variant in the transformed host cell, wherein the host cell is transformed with the expression vector comprising the nucleic acid sequence encoding the glucanase variant of SEQ ID NO: 19.

The present invention provides a method for aiding or supporting digestion of beta-glucan in a subject, said method comprising administering to the subject a pharmaceutical composition comprising the glucanase variant of SEQ ID NO: 19.

In an embodiment of the method for aiding or supporting digestion of beta-glucan in a subject, the glucanase variant improves digestion of beta-glucans in oats, wheat, barley, rye, wheat, sorghum, reishi mushrooms, shiitake mushrooms, maitake mushrooms, yeasts, seaweed or algae compared to digestion upon administration of a pharmaceutical composition that does not comprise the glucanase variant.

In another embodiment of the method the aiding or supporting digestion of beta-glucan in a subject, the glucanase variant decreases malabsorption, bloating, abdominal discomfort and gas compared to digestion upon administration of a pharmaceutical composition that does not comprise the glucanase variant.

The present invention also provides a method for treating overgrowth of a fungus or an overabundance of beta-glucan in a digestive tract of a subject, said method comprising administering to the subject a pharmaceutical composition comprising the glucanase variant of SEQ ID NO: 19.

In an embodiment of said method, the glucanase variant reduces biofilm produced by the fungus.

In another embodiment of said method, said fungus is a Candida species.

In a further embodiment of said method, the Candida species is Candida albicans, Candida tropicalis, and/or Candida dubliniensis.

The present invention further provides a method for preparing a functional food or functional animal feed, said method comprising pre-digesting with the glucanase variant of SEQ ID NO: 19 beta-glucan containing cereal selected from oats, wheat, barley, rye, wheat and sorghum, thereby preparing the functional food or functional animal feed.

In an embodiment of said method, the functional food or functional animal feed enhances digestion and absorption of the food or feed in a subject compared to digestion and absorption of food or feed which has not been pre-digested with the glucanase variant.

The present invention provides a method for aiding in the color and texture of wine, said method comprising aging wine with the glucanase variant of SEQ ID NO: 19.

The present invention also provides a method of treating waste, said method comprising treating the waste with the glucanase variant of SEQ ID NO: 19.

In an embodiment of said method of treating waste, the waste comprises cellulose.

In an embodiment of said method of treating waste, said method further comprises treating the waste with the above-described cellulase variant of SEQ ID NO: 19.

In an embodiment of said method of treating waste, the waste comprises containing oats, wheat, barley, rye, wheat, and/or sorghum.

### Glucose Isomerase Variants

Glucose isomerase, also called xylose isomerase, is an enzyme that converts glucose to fructose. Glucose isomerase also catalyzes the isomerization of xylose to xylulose.

Glucose isomerase is used in the food industry to produce high-fructose corn syrup, which is an equilibrium mixture of glucose and fructose that is 1.3 times sweeter than sucrose. The use of glucose isomerase results in a higher yield of fructose with no by-products than older fructose production methods. Glucose isomerase is inactivated at high temperatures, however, high temperatures, over 70° C., increase the yield of fructose by half. Glucose isomerase requires a high pH of between 7.0 and 90° C. for the isomerization reaction.

The glucose isomerase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type glucose isomerase will denature, decompose and/or under which the glucose isomerase enzymatic function is significantly reduced or eliminated.

The present invention provides a glucose isomerase variant of a wild-type glucose isomerase from Streptomyces murinus of SEQ ID NO: 20 comprising mutations:
a) T39Y, Y49W, T85K, T119V, I120M, G121R, A136F, G146L, D153R, A171K, D190H, E204Y, G231E, A301E, I312A, R316K, D348T, and L383T;
b) F3Y, R10K, T39Y, Y49W, G62D, T66A, S70K, R76K, A81D, T85K, A106S, T119V, I120M, G121R, I123L, A136M, G146L, D153R, G166A, E167D, A171E, D190H, L192Y, V196T, E204Y, Y212F, L226H, G231E, A238H, A301E, I312A, D315E, R316K, A318I, A319K, Q337E, D348A, A355S, H374K, and L383T;
c) T39Y, Y49W, T85K, I120M, G121R, A136F, G146L, D153R, A171K, D190H, E204Y, G231E, A301E, I312A, R316K, D348T, and L383T;
d) F3Y, T39Y, Y49W, A81D, T85K, T119V, I120M, G121R, A136F, G146L, D153R, A171E, D190H, E204Y, Y212F, G231E, A301E, I312A, D315E, R316K, A319K, D348A, A355S, and L383T;
e) T39I, Y49W, T85K, T119V, I120M, G121R, A136F, A171K, D190H, E204Y, G231E, A301E, R316K, D348T, and L383T;
f) F3Y, R10K, T39Y, Y49W, G62D, Q77D, A81D, T85K, T119V, I120M, G121R, I123L, A136F, A143F, D153R, A171E, D190H, Y212F, G231E, A301E, I312A, D315E, R316K, A319K, D348A, A355S, H374W, and L383T;
g) F3Y, T39Y, Y49W, A81D, T85K, T119V, I120M, G121R, A136F, D153R, A171E, D190H, Y212F, G231E, A301E, I312A, D315E, R316K, A319K, D348A, A355S, and L383T;
h) T39Y, Y49W, T85K, T119V, I120M, G121R, A136M, G146L, A171K, D190H, E204Y, G231E, A301E, R316K, D348T, and L383T;
i) F3Y, T39Y, Y49W, T85K, T119V, I120M, G121R, A136F, G146L, D153R, A171E, D190H, E204Y, G231E, A301E, I312A, R316K, D348A, A355S, and L383T;
j) F3Y, R10K, T39Y, Y49W, G62D, T66A, S70K, R76K, A81D, T85K, A106S, I120M, G121R, A136M, A143T, G146L, D153R, G166A, E167D, A171E, L176I, D190H, L192Y, V196T, Y212F, L226H, G231E, A238H, A301E, I312A, R316K, A318R, Q337E, D348A, A355S, H374K, and L383T;
k) F3Y, R10K, T39Y, Y49W, G62D, T66A, S70K, R76K, A81D, T85K, A89V, A106S, T119V, I120M, G121R, I123L, A136F, D153R, G166A, A171E, L176I, D190H, L192Y, V196A, Y212F, G231E, A238H, A301E, I312A, D315E, R316K, A318R, D348A, A355S, H374K, and L383T;
l) F3Y, R10K, T39Y, G62D, T66A, S70K, R76K, A81D, T85K, A89V, A106S, T119V, I120M, G121R, I123L, A136F, D153R, G166A, A171E, L176I, D190H, L192Y, Y212F, G231E, A238H, A301E, I312A, D315E, R316K, A318R, D348A, A355S, H374K, and L383T; or
m) F3Y, R10K, T39Y, G62D, Q77D, A81D, T85K, A89V, T119V, I120M, G121R, I123L, A136F, A143F, D153R, A171E, L176I, D190H, V196I, Y212F, A301E, I312A, D315E, R316K, A319K, D348A, A355S, H374W, and L383T.

In an embodiment, the present invention provides a nucleic acid sequence encoding the above-described glucose isomerase variant of SEQ ID NO: 20.

In another embodiment of the present invention, an expression vector comprises the nucleic acid sequence encoding the glucose isomerase variant of SEQ ID NO: 20.

In an embodiment, the present invention provides a host cell transformed with the expression vector comprising the nucleic acid sequence encoding the glucose isomerase variant of SEQ ID NO: 20.

In a further embodiment, the present invention provides a method for producing a glucose isomerase variant, said method comprising expressing the glucose isomerase variant in the transformed host cell, wherein the host cell is transformed with the expression vector comprising the nucleic acid sequence encoding the glucose isomerase variant of SEQ ID NO: 20.

The present invention also provides a method for isomerizing glucose to fructose, said method comprising
a) treating corn starch with an alpha-amylase to produce oligosaccharides;
b) treating the oligosaccharides of step (a) with glucomaylase to produce glucose;
c) treating the glucose of step (b) with glucose isomerase variant of SEQ ID NO: 20 at a temperature of 60-90° C. to produce a mixture of glucose and fructose, wherein said mixture is a high-fructose corn syrup.

In an embodiment of the method for isomerizing glucose to fructose, the temperature in step (c) is 60-70° C.

In another embodiment of the method for isomerizing glucose to fructose, the glucose isomerase variant is immobilized.

### Inulase Variants

Inulase, also called inulinase or beta-fructofuranosidase, is an enzyme that catalyzes endohydrolysis of (2→1)-beta-D-fructosidic linkages in inulin, thereby converting inulin to levulose, a fructose isomer. Inulin, a polysaccharide in the class of dietary fiber known as fructans, is produced by many plants and some microorganisms, including, mold, such as Aspergillus niger. Inulin-containing plants include chicory, Jerusalem artichoke, dahlia, leek onion and garlic.

Additional industrial interest in inulase relates to the production of a low-calorie sugar substitute, difructose anhydride III, the smallest cyclic disaccharide consisting of two fructose residues, which is a hydrolysate of inulin. However, difructose anhydride III is rarely found in nature.

Plants containing inulin are carbohydrate substrates for acetone-butanol-ethanol fermentation by Clostridia. Inulase is used to hydrolyze the fructans before fermentation. Studies have shown that the yeast Klyveromyces marxianus, which produces inulase, hydrolyzes inulin to fructose and then ferments the fructose to produce ethanol.

The inulase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type inulase will denature, decompose and/or under which the inulase enzymatic function is significantly reduced or eliminated.

The present invention provides an inulase variant of a wild-type inulase from Aspergillus niger of SEQ ID NO: 21 comprising mutations:
a) Q38A, S52G, I70M, A84D, A91P, T100S, R183E, D198H, N293Y, Q350R, H351R, A400P, S460H, D489P, and N502S;
b) S25R, Q38A, S52G, I70M, A84D, A91P, T100S, S132Q, Q133G, R183E, N187R, D198H, A206T, N293Y, V304E, V346I, Q350R, H351R, A400P, E453P, S460H, D489P, and N502S;
c) S25R, D37K, Q38A, S52G, N69P, I70M, A84E, A91P, D92S, T100S, D115T, A117T, S132Q, Q133G, V143T, R183E, N187K, D198N, A206D, S221Q, E246N, A286P, N293Y, V304E, V346I, Q350R, H351R, T358S, A371Q, A395K, A400P, K450Q, E453P, G455S, S460H, Q476D, D489E, N502S, S507C, and S512Y;
d) Q38A, S52G, I70M, A84D, A91P, T100S, R183E, D198H, A281P, N293Y, V346I, Q350R, H351R, A400P, S460H, and N502S;
e) S25R, D37K, Q38A, S52G, W54Y, T63Y, I70M, M81L, A84D, A91P, D92S, T100S, D115T, A117D, T129N, S132Q, Q133G, V143T, R183E, Q184E, N187K, D198N, A206T, S221Q, E246N, A286P, N293Y, V304E, T329N, V346I, Q350R, H351R, T358S, A371T, A395E, A400P, A412G, A425N, K450T, E453P, G455S, S460H, Q476D, D489E, N502S, S507C, and S512Y;
f) S25R, Q38A, S52G, I70M, A84D, A91P, T100S, S132Q, Q133G, R183E, N187R, D198H, A206T, A281P, N293Y, V304E, V346I, Q350R, H351R, A400P, E453P, S460H, D489P, and N502S;
g) S25R, D37K, Q38A, S52G, T63Y, I70M, A84E, A91P, D92S, T100S, D115T, A117T, S132Q, Q133G, V143T, R183E, N187K, D198N, A206T, S221Q, E246N, A286P, N293Y, V304E, V346I, Q350R, H351R, T358S, A371Q, A395K, A400P, K450Q, E453P, G455S, S460H, Q476D, D489E, N502G, S507C, and S512Y;
h) S25T, D37K, Q38A, S52G, W54Y, T63Y, I70M, A84E, A91P, D92S, T100S, D115T, A117K, T129N, S132Q, Q133G, V143T, R183E, Q184E, N187K, D198N, A206T, S221Q, E246N, A286P, N293Y, V304E, T329N, Q350R, H351R, T358S, S364G, A371T, A395K, A400P, A412G, A425N, K450T, E453P, G455S, S460H, Q476D, D489E, N502S, S507C, S512Y, and V513I;
i) S25R, D37K, Q38A, S52G, T63Y, I70M, A84D, A91P, D92S, T100S, D115T, A117T, S132Q, Q133G, R183E, N187K, D198H, A206D, S221Q, E246N, A286P, N293Y, V304E, V346I, Q350R, H351R, A371Q, A395K, A400P, E453P, S460H, D489E, N502S, S507C, and S512Y;
j) S25R, D37K, Q38A, S52G, N69P, I70M, A84D, A91P, T100S, D115T, A117T, S132Q, Q133G, R183E, N187Q, D198H, A206T, S221Q, A286P, N293Y, V304E, V346I, Q350R, H351R, A400P, E453P, S460H, D489E, N502S, and S512Y;
k) S25R, D37K, Q38A, S52G, T63Y, I70M, A84D, A91P, D92S, T100S, D115T, A117T, S132Q, Q133G, R183E, N187K, D198H, A206T, S221Q, E246N, A286P, N293Y, V304E, V346I, Q350R, H351R, A371Q, A395K, A400P, E453P, S460H, D489E, N502S, S507C, and S512Y;
l) S25R, D37K, Q38A, S52G, T53V, W54Y, F58Y, T63Y, I70M, A84E, T88V, A91P, D92S, T100S, T102S, D115T, A117K, T129N, T130K, S132Q, Q133G, F141Y, V143T, T150V, R183E, Q184E, N187K, G196S, D198N, A206D, T220S, S221Q, E246N, A286P, N293Y, V304E, T329N, S342Q, V346I, Q350R, H351R, T358S, A371T, A395K, A400P, A412G, T421R, A425N, K450T, E452S, E453P, G455S, S460H, Q476D, D489E, N502S, S507C, and S512Y;
m) S25R, D37K, Q38A, S52G, N69P, I70M, A84D, A91P, T100S, D115T, A117T, S132Q, Q133G, R183E, N187Q, D198H, A206T, S221Q, A286P, N293Y, V304E, Q350R, H351R, A400P, E453P, S460H, D489E, N502S, and S512Y; or
n) S25R, D37K, Q38A, S52G, T53V, W54Y, F58Y, T63Y, I70M, M81L, A84D, T88V, A91P, D92S, T100S, T102S, D115T, A117K, T129N, T130K, S132Q, Q133G, F141Y, V143T, T150V, R183E, Q184E, N187K, G196S, D198N, A206T, T220S, S221Q, E246N, A286P, N293Y, V304E, T329N, S342Q, V346I, Q350R, H351R, T358S, A371T, A395K, A400P, A412G, T421R, A425N, K450T, E452S, E453P, G455S, S460H, Q476D, D489E, N502G, S507C, and S512Y.

In an embodiment, the present invention provides a nucleic acid sequence encoding the above-described inulase variant of SEQ ID NO: 21.

In another embodiment of the present invention, an expression vector comprises the nucleic acid sequence encoding the inulase variant of SEQ ID NO: 21.

In an embodiment, the present invention provides a host cell transformed with the expression vector comprising the nucleic acid sequence encoding the inulase variant of SEQ ID NO: 21.

In a further embodiment, the present invention provides a method for producing an inulase variant, said method comprising expressing the inulase variant in the transformed host cell, wherein the host cell is transformed with the expression vector comprising the nucleic acid sequence encoding the inulase variant of SEQ ID NO: 21.

The present invention provides a method for preparing fructose, said method comprising hydrolyzing inulin with the inulase variant of SEQ ID NO: 21.

The present invention also provides a method for producing ethanol, said method comprising fermenting an inulin source with the inulase variant of SEQ ID NO: 21.

### Lactase Variants

Lactase is an enzyme that hydrolyzes lactose into galactose and glucose monomers. Lactose is a disaccharide found in milk. Lactase is produced by humans and other mammals; its optimum temperature is 37° C. and its optimum pH is 6. Lactase is a food supplement and also is added to milk to make lactose-free milk for consumption by people are lactose intolerant due to a lactase deficiency.

The lactase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type lactase will denature, decompose and/or under which the lactase enzymatic function is significantly reduced or eliminated.

The present invention provides a lactase variant of a wild-type lactase from Kluyveromyces of SEQ ID NO: 22 comprising mutations:
a) N36S, A42G, K62S, V69A, K80S, N88S, T112S, E114S, D116P, K118D, I120S, N134S, L138V, K149Q, E167D, S180G, K202A, S278C, K308H, V371I, K379Q, F380H, N387C, S388A, F400C, I408M, T415C, V455P, S462E, L464M, V469K, C484G, L514E, S562G, E573Y, K574P, Y576L, L770M, N835R, S839A, V842G, Y844A, K852T, A857G, G887S, E946Q, E957D, and E999A;
b) N36S, A42G, K62S, V69A, K80S, N88S, T112S, E114S, D116P, K118D, I120S, N134S, L138V, K149Q, E167D, S180G, K202A, S278C, K308H, V371I, K379Q, F380H, N387C, S388A, F400C, I408M, T415C, V455P, S462E, L464M, V469K, C484G, L514E, S562G, E573Y, K574P, Y576L, N835R, S839A, V842G, Y844A, K852T, A857G, G887S, E946Q, E957D, and E999A;
c) N36S, F41C, A42G, K62S, V69A, S71G, T112S, E114S, D116P, I120K, N134S, F175Y, S180G, K202A, V371I, K379Q, N387C, T415C, V455K, S462E, V469K, C484G, L514E, S562G, E573Y, Y576L, L770M, N835R, V842G, Y844A, G887S, and E957D;
d) N36S, A42G, K62S, V69A, K80S, N88S, T112S, E114S, D116P, K118D, I120S, N134S, L138V, K149Q, E167D, F175Y, K176Q, S180G, K202A, S278C, K308H, V371I, K379Q, F380H, N387C, S388A, F400C, I408M, T415C, D441K, Y446W, V455P, S462E, L464M, V469K, C484G, L514E, F526V, S562G, E573Y, K574D, Y576L, I581V, E768Q, S779T, N835R, S839A, V842G, Y844A, K852T, A857G, N861K, G887S, Y916F, E946Q, E957D, and E999A;
e) N36S, F41C, A42G, K62S, V69A, E74S, K80S, N88S, T112S, E114S, D116P, K118D, I120S, E121D, N134S, L138V, N148A, K149Q, I159V, E167D, K173R, F175Y, K176Q, S180G, K202A, T216V, S278C, K308H, V371I, K379Q, F380H, N387C, F400C, I408M, T415C, N423A, K438L, D441K, V442E, H445K, Y446W, V455P, S462E, L464M, V469K, C484G, N512H, L514E, F526I, H536E, S562G, E573Y, K574D, Y576L, V701S, E768Q, S779T, I816V, V832I, T833I, N835R, S839A, V841A, V842G, Y844A, K852T, A857G, N861K, N863R, S865T, G887S, Y916F, E946Q, E957D, and E999A;
f) N36S, F41C, A42G, A50V, K62S, V69A, E74S, K80S, N88S, T112S, E114S, D116P, K118D, I120S, E121D, N134S, L138V, N148A, K149Q, I159V, E167D, K173R, F175Y, S180G, K202A, T216V, S278C, K308H, V371I, K379Q, F380H, N387T, S388A, F400C, I408M, T415C, N423A, K438L, D441K, V442E, H445K, Y446W, V455P, S462E, L464M, V469K, C484G, L514E, F526I, H536E, S562G, E573Y, K574D, Y576L, V701S, L770M, S779T, I816V, V832I, T833I, N835R, S839A, V841A, V842G, Y844A, K852T, A857G, N861K, N863R, S865T, G887S, Y916F, E946Q, E957D, and E999A;
g) N36G, F41C, A42G, A50V, K62S, V69A, E74S, K80S, N88S, T112S, E114S, D116P, K118D, I120K, N134S, L138V, N148A, K149Q, E167D, K176Q, S180G, K202A, S278C, K308H, V371I, K379Q, F380H, N387C, S388A, F400C, I408M, T415C, K438L, D441K, H445K, Y446W, V455P, S462E, L464M, V469K, C484G, L514E, F526I, S562G, E573Y, K574D, Y576L, L770M, S779T, T833I, N835R, S839A, V842G, Y844A, K852T, A857G, N861K, S865T, G887S, Y916F, E946Q, E957D, and E999A;
h) N36S, F41C, A42G, K62S, V69A, S71G, N88S, T112S, E114S, D116P, K118D, I120S, N134S, L138V, K149Q, E167D, F175Y, S180G, K202A, S278C, V371I, K379Q, F380H, N387C, S388A, F400C, I408M, T415C, V455P, S462E, L464M, V469K, C484G, L514E, S562G, E573Y, K574P, Y576L, N835R, S839A, V842G, Y844A, A857G, G887S, and E957D;
i) N36S, A42G, K62S, V69A, S71G, N88S, T112S, E114S, D116P, K118D, I120S, N134S, K149Q, E167D, F175Y, S180G, K202A, S278C, V371I, K379Q, F380H, N387C, S388A, F400C, I408M, T415C, V455P, S462E, L464M, V469K, C484G, L514E, S562G, E573Y, K574P, Y576L, L770M, N835R, S839A, V842G, Y844A, A857G, G887S, and E957D;
j) N36S, F41C, A42G, A50V, K62S, V69A, S71G, E74S, K80S, N88S, T112S, E114S, D116P, K118D, I120K, N134S, L138V, N148A, K149Q, E167D, K176Q, S180G, K202A, S278C, K308H, V371I, K379Q, F380H, N387C, S388A, F400C, I408M, T415C, K438L, D441K, H445K, Y446W, V455P, S462E, L464M, V469K, C484G, L514E, F526I, S562G, E573Y, K574D, Y576L, E768Q, S779T, T833I, N835R, S839A, V842G, Y844A, K852T, A857G, N861K, S865T, G887S, Y916F, E946Q, E957D, and E999A;
k) N36S, F41C, A42G, K62S, V69A, S71G, T112S, E114S, D116P, I120S, N134S, S180G, K202A, K379Q, N387C, T415C, V455K, S462E, V469K, C484G, L514E, S562G, E573Y, Y576L, N835R, V842G, and G887S;
l) N36G, A42G, K62S, V69A, T112S, E114S, D116P, I120S, N134S, F175Y, S180G, K202A, V371I, K379Q, N387C, T415C, V455E, S462E, V469K, C484G, L514E, S562G, E573Y, Y576L, N835R, V842G, Y844A G887S, and E957D; or
m) N36S, F41C, A42G, A50V, K62S, V69A, K80S, N88S, T112S, E114S, D116P, K118D, I120S, N134S, L138V, K149Q, E167D, K176Q, S180G, K202A, S278C, K308H, V371I, K379Q, F380H, N387T, S388A, F400C, I408M, T415C, D441K, Y446W, V455P, S462E, L464M, V469K, C484G, N512H, L514E, F526V, S562G E573Y, K574D, Y576L, E768Q, S779T, N835R, S839A, V842G, Y844A, K852T, A857G, N861K, G887S, Y916F, E946Q, E957D, and E999A.

In an embodiment, the present invention provides a nucleic acid sequence encoding the above-described lactase variant of SEQ ID NO: 22.

In another embodiment of the present invention, an expression vector comprises the nucleic acid sequence encoding the lactase variant of SEQ ID NO: 22.

In an embodiment, the present invention provides a host cell transformed with the expression vector comprising the nucleic acid sequence encoding the lactase variant of SEQ ID NO: 22.

In a further embodiment, the present invention provides a method for producing a lactase variant, said method comprising expressing the lactase variant in the transformed host cell, wherein the host cell is transformed with the expression vector comprising the nucleic acid sequence encoding the lactase variant of SEQ ID NO: 22.

The present invention provides a pharmaceutical composition comprising the lactase variant of SEQ ID NO: 22.

The present invention also provides method for treating lactose intolerance or lactose deficiency in a subject, said method comprising administering to the subject a pharmaceutical composition comprising the lactase variant of SEQ ID NO: 22.

### Lysozyme Variants

Lysozyme is an enzyme that hydrolyzes 1, 4-beta-linkages between N-acetylmuramic acid and N-acetyl-D-glucosamine residues in peptidoglycan, the major component of gram-positive bacterial cell wall, and thus is an anti-microbial enzyme.

Lysozyme is secreted in tears, saliva, human milk, and mucus. Lysozyme also is found in egg whites. In addition to its antibacterial activity, lysozyme has been found to have antiviral and anti-tumor activity. Lysozyme can be used as a preservative in various food and beverages, including cheese and wine, and has been tested as a preservative for meat products, infant dry milk, tofu and other food products. Lysozyme loses some of its activity at high temperature of 60° C. or above, or prolonged storage.

The lysozyme variants provided herein are stable under conditions such as high temperature, e.g., over 50° C., pH extremes, e.g., high acidity at pH of 4 or lower, in the presence of salts, alkalis and/or surfactants, and other such severe conditions under which wild-type lysozyme will denature, decompose and/or under which the lysozyme enzymatic function is significantly reduced or eliminated.

The present invention provides a lysozyme variant of a wild-type lysozyme from chicken of SEQ ID NO: 23 comprising mutations:
a) G22T, A28R, N37G, G44A, K51Y, F52Y, S109A, N111E, R132H, T136H, Q139S, A140K, and R146D;
b) G22T, A28R, N37G, G44A, K51Y, F52H, S109A, R132H, T136R, Q139S, A140K, and R146D;
c) K51Y, R132H, and Q139S;
d) N37G, G44A, K51Y, F52H, S109A, R132H, and Q139S;
e) G22T, A28R, N37G, R39K, G44A, K51Y, F52Y, S90A, S109A, N111E, V127D, R132H, T136H, Q139S, A140K, and R146D;
f) G44A, K51Y, S109A, R132H, and Q139S;
g) F21Y, A28R, N37G, R39K, G44A, K51Y, F52Y, S90A, R91K, S109A, N111E, V127D, R132H, T136H, Q139S, A140K, and R146D;
h) F21Y, A28R, N37G, R39K, K51Y, F52Y, S90A, R91K, S104D, S109A, N111E, V127D, R132H, T136H, Q139S, A140K, and R146D; or
i) G22T, A28K, N37G, R39K, G44A, K51Y, F52Y, S90A, S109A, N111E, V127D, R132H, T136K, Q139S, A140K, and R146D.

In an embodiment, the present invention provides a nucleic acid sequence encoding the above-described lysozyme variant of SEQ ID NO: 23.

In another embodiment of the present invention, an expression vector comprises the nucleic acid sequence encoding the lysozyme variant of SEQ ID NO: 23.

In an embodiment, the present invention provides a host cell transformed with the expression vector comprising the nucleic acid sequence encoding the lysozyme variant of SEQ ID NO: 23.

In a further embodiment, the present invention provides a method for producing a lysozyme variant, said method comprising expressing the lysozyme variant in the transformed host cell, wherein the host cell is transformed with the expression vector comprising the nucleic acid sequence encoding the lysozyme variant of SEQ ID NO: 23.

The present invention also provides a pharmaceutical composition comprising the lysozyme variant of SEQ ID NO: 23.

The present invention further provides a method for treating a wound, said method comprising administering to the wound the pharmaceutical composition of SEQ ID NO: 23.

In an embodiment of the method for treating a wound, said treatment is effective against gram-positive bacteria.

In an embodiment of the method for treating a wound, the gram-positive bacteria is a Streptococci species or Staphylococci species.

The present invention also provides a method for treating conjunctivitis in an eye of a subject, said method comprising administering to the eye the pharmaceutical composition comprising the lysozyme variant of SEQ ID NO: 23.

The present invention further provides a method for treating a bladder infection in a subject, said method comprising administering to the subject the pharmaceutical composition comprising the lysozyme variant of SEQ ID NO: 23.

In an embodiment of the method for treating a bladder infection in a subject, the bladder infection is an infection by gram-negative bacteria.

In another embodiment of the method for treating a bladder infection in a subject, the gram-negative bacteria is an Escherichia species, a Klebsiella species, an Enterobacter species, a Pseudomonas species, a Citrobacter species, and/or a Proteus species.

In another embodiment of the method for treating a bladder infection in a subject, the Escherichia species is Escherichia coli, the Klebsiella species is K. pneumoniae, an Enterobacter species is E. aerogenes, the Pseudomonas species is P. aeruginosa, the Citrobacter species is C. koseri, and the Proteus species is P. mirabillis.

The present invention provides a method for treating inflammation in a subject, said method comprising administering to the subject a pharmaceutical composition comprising the lysozyme variant of SEQ ID NO: 23.

In an embodiment of any of the above-described methods for treating a subject, said methods further comprise administering to the subject a second pharmaceutical composition, said second pharmaceutical composition comprising an antibiotic.

The present invention also provides a method for increasing body weight of an infant, said method comprising administering to the infant a pharmaceutical composition comprising the lysozyme variant of SEQ ID NO: 23.

In an embodiment of the method for increasing body weight of an infant, the infant is human.

In an embodiment of the method for increasing body weight of an infant, the infant is a premature infant.

In an embodiment of any of the above-described methods for increasing body weight of an infant, the pharmaceutical composition comprising the lysozyme variant of SEQ ID NO: 23 is added to human breast milk or to infant formula.

The present invention provides a food or beverage additive comprising the lysozyme variant of SEQ ID NO: 23.

The present invention also provides a method for preserving a food or a beverage, said method comprising treating the food or beverage with the food or beverage additive comprising the lysozyme variant of SEQ ID NO: 23, wherein the lysozyme variant improves preservation of the food or beverage compared to preservation of the food or beverage with a preservative that does not comprise the lysozyme variant.

In an embodiment of said method for preserving a food or a beverage, the food is cheese and the beverage is wine.

### Pectin Lyase Variants

Pectin lyase is an enzyme that degrades pectin. In the food industry, pectin lyase is used to degrade highly esterified pectin in fruit, wine and cider, without producing methanol. Pectin lyase also is used to clarify fruit juices.

Alkaline pectinase is used in the textile industry for retting of plant fibers, such as flax, hemp, ramie, and jute.

The pectin lyase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type pectin lyase will denature, decompose and/or under which the pectin lyase enzymatic function is significantly reduced or eliminated.

The present invention provides a pectin lyase variant of a wild-type pectin lyase from Aspergillus niger SEQ ID NO: 24 comprising mutations:
a) H32S, T77S, A84C, Q93C, H99N, S100N, D103C, I121T, T137S, K153E, D179N, L215D, R219A, D221S, N227C, Y235M, M242Q, N252H, L253T, M257G, K259A, Q261G, F276N, I283V, Q310H, A324C, Q332C, T361A, N367A, T372V, and M373L;
b) V24S, H32S, D49A, T77S, A84C, Q91A, Q93C, L98Q, H99N, S100N, D103C, K118N, I121T, T137S, K153E, A161H, K168Q, V178I, D179N, S214N, L215D, R219N, D221S, N227C, Q229H, Y235M, L236F, M242Q, F250I, N252H, L253T, M257G, K259D, Q261G, L272V, F273W, H274Y, F276N, Q310H, S314N, A324C, Q332C, T361A, T372A, and Q378A;
c) V24Q, H32S, T77S, A84C, Q91A, Q93C, L98Q, H99N, S100N, D103C, I121T, T137S, K153E, A161H, K168Q, D179N, L215D, R219A, D221S, N227C, Q229H, Y235M, M242Q, F250I, N252H, L253T, M257G, K259D, Q261G, L272V, F273W, H274Y, F276N, I283V, Q310H, A324C, Q332C, T361A, N367A, T372A, and Q378A;
d) V24S, D49A, T77S, A84C, Q91A, Q93C, L98K, H99N, S100N, D103C, T137S, V140A, V148I, V178I, D179N, Y213N, L215D, I216F, N227C, Q229H, N252H, L253S, M257G, K259A, N275Q, F276N, A291I, I307L, A324C, Q332C, S339N, T361A, N367M, T372V, and Q378A;
e) A84C, Q93C, D103C, N227C, A324C, Q332C, T361A, and N367A;
f) H32S, T77S, A84C, Q93C, H99D, S100N, D103C, T137S, L215D, D217Y, R219A, N227C, Y235L, M242Q, N252H, L253T, M257G, K259A, Q261G, F276N, Q310H, A324C, Q332C, T361A, N367A, and T372V;
g) H32S, T77S, A84C, Q93C, H99D, S100N, D103C, T137S, V140A, L215D, D217Y, R219A, N227C, Y235L, M242Q, N252H, L253T, M257G, Q261G, F276N, Q310H, A324C, Q332C, T361A, N367A, and T372V;
h) H32S, T77S, A84C, Q91A, Q93C, H99N, S100N, D103C, I121T, T137S, V140A, K153E, A161H, D179N, L215D, R219A, D221S, N227C, Q229H, Y235M, M242Q, F250V, N252H, L253T, M257G, K259D, Q261G, L272V, F273W, F276N, I283V, Q310H, A324C, Q332C, T361A, N367A, and T372A;
i) H32S, T77S, A84C, Q93C, H99D, S100N, D103C, I121T, T137S, V140A, L215D, R219A, D221S, N227C, Y235M, M242Q, N252H, L253T, M257G, K259D, Q261G, F276N, Q310H, A324C, Q332C, T361A, N367A, and T372V;
j) T77S, A84C, Q93C, H99N, S100N, D103C, T137S, V140A, D179N, Y213N, N227C, Q229H, N252H, L253S, M257G, F273W, F276N, A324C, Q332C, T361A, and N367A;
k) V24S, H32S, D49A, T77S, A84C, Q91A, Q93C, L98Q, H99N, S100N, D103C, K118N, I121T, T137S, K153E, A161H, K168Q, V178I, D179N, S214N, L215D, R219N, D221S, N227C, Q229H, Y235M, L236F, M242Q, F250I, N252H, L253T, M257G, K259A, Q261G, L272V, F273W, H274Y, F276N, I283V, Q310H, S314N, A324C, Q332C, T361A, T372A, and Q378A;
l) H32S, T77S, A84C, Q93C, H99N, S100N, D103C, T137S, V140A, K153E, D179N, L215D, R219A, D221S, N227C, Y235M, M242Q, N252H, L253T, M257G, K259A, Q261G, F276N, I283V, Q310H, A324C, Q332C, T361A, N367A, T372V, and M373L;
m) D49A, T77S, A84C, Q93C, L98K, H99N, S100N, D103C, T137S, V140A, V148I, D179N, Y213N, L215D, N227C, Q229H, N252H, L253S, M257G, K259A, F273W, F276N, A324C, Q332C, T361A, N367A, and T372V;
n) V24S, H32S, D49A, N58S, T77S, A84C, Q91A, Q93C, L98Q, H99N, S100N, D103C, K118N, I121T, T137S, K153E, A161H, V162I, K168Q, V178I, D179N, Y213N, S214N, L215D, R219N, D221S, N227C, Q229H, Y235M, L236F, M242Q, F250I, N252H, L253T, M257G, K259D, Q261G, L272V, F273W, H274Y, F276N, I283V, A291L, Q310H, S313N, S314N, A324C, Q332C, T361A, T372A, and Q378A;
o) V24S, D49A, T77S, A84C, Q91A, Q93C, L98K, H99N, S100N, D103C, T137S, V140A, V148I, V178I, D179N, Y213N, L215D, N227C, Q229H, N252H, L253S, M257G, K259A, F273W, N275Q, F276N, A291I, I307L, A324C, Q332C, S339N, T361A, N367M, T372V, and Q378A;
p) T77S, A84C, Q93C, H99N, S100N, D103C, T137S, V140A, D179N, Y213N, N227C, N252H, L253S, M257G, F276N, A324C, Q332C, T361A, and N367A;
q) V24Q, H32S, T77S, A84C, Q91A, Q93C, L98Q, H99N, S100N, D103C, I121T, T137S, K153E, A161H, K168Q, D179N, L215D, R219A, D221S, N227C, Q229H, Y235M, M242Q, F250I, N252H, L253T, M257G, K259A, Q261G, L272V, F273W, H274Y, F276N, I283V, Q310H, A324C, Q332C, T361A, N367A, T372A and, Q378A;
r) V24T, D49A, N58S, T77S, A84C, Q91A, Q93C, L98K, H99N, S100N, D103C, T137S, V140A, V148I, A161H, V162I, I165L, V178I, D179N, Y213N, L215D, N227C, Q229H, N252H, L253S, M257G, K259A, N275S, F276N, D277N, A291I, I307L, A324C, Q332C, S339N, T361A, Q370E, T372V, and Q378A;
s) V24S, D49A, N58S, T77S, A84C, Q91A, Q93C, L98K, H99N, S100N, D103C, T137S, V140A, V148I, A161H, V162I, I165L, V178I, D179N, Y213N, L215D, I216F, N227C, Q229H, N252H, L253S, M257G, K259A, F273W, N275Q, F276N, D277N, A291I, I307L, A324C, Q332C, S339N, T361A, N367R, Q370E, T372V, and Q378A;
t) V24S, D49A, T77S, A84C, Q91A, Q93C, L98K, H99N, S100N, D103C, T137S, V140A, V148I, V178I, D179N, Y213N, L215D, N227C, Q229H, N252H, L253S, M257G, K259A, F273W, N275Q, F276N, A324C, Q332C, T361A, N367A, T372V, and Q378A;
u) V24S, H32S, D49A, N58S, T77S, A84C, Q91A, Q93C, L98Q, H99N, S100N, D103C, K118N, I121T, T137S, K153E, A161H, V162I, K168Q, V178I, D179N, Y213N, S214N, L215D, R219N, D221S, N227C, Q229H, Y235M, L236F, M242Q, F250I, N252H, L253T, M257G, K259A, Q261G, L272V, F273W, H274Y, F276N, I283V, A291L, Q310H, S313N, S314N, A324C, Q332C, T361A, T372A, and Q378A;
v) H32S, T77S, A84C, Q93C, H99D, S100N, D103C, I121T, T137S, V140A, L215D, R219A, D221S, N227C, Y235M, M242Q, N252H, L253T, M257G, K259A, Q261G, F276N, Q310H, A324C, Q332C, T361A, N367A, and T372V;
w) H32S, T77S, A84C, Q91A, Q93C, H99N, S100N, D103C, T137S, A161H, D179N, L215D, R219A, D221S, N227C, Q229H, Y235M, M242Q, F250V, N252H, L253T, M257G, K259A, Q261G, L272V, F273W, F276N, I283V, Q310H, A324C, Q332C, T361A, N367A, and T372A; or
x) T77S, A84C, Q93C, H99N, S100N, D103C, T137S, V140A, D179N, Y213N, L215D, N227C, Q229H, N252H, L253S, M257G, K259A, F273W, F276N, A324C, Q332C, T361A, N367A, and I368Q.

In an embodiment, the present invention provides a nucleic acid sequence encoding the above-described pectin lyase variant of SEQ ID NO: 24.

In another embodiment of the present invention, an expression vector comprises the nucleic acid sequence encoding the pectin lyase variant of SEQ ID NO: 24.

In an embodiment, the present invention provides a host cell transformed with the expression vector comprising the nucleic acid sequence encoding the pectin lyase variant of SEQ ID NO: 24.

In a further embodiment, the present invention provides a method for producing a pectin lyase variant, said method comprising expressing the pectin lyase variant in the transformed host cell, wherein the host cell is transformed with the expression vector comprising the nucleic acid sequence encoding the pectin lyase variant of SEQ ID NO: 24.

The present invention also provides a method of degrading pectin in a beverage, said method comprising treating the beverage with the pectin lyase variant of SEQ ID NO: 24.

In an embodiment of the method of degrading pectin in a beverage, the beverage is a wine, cider or fruit juice.

The present invention further provides a method for retting of plant fibers, said method comprising treating the plant with a composition comprising the pectin lyase variant of SEQ ID NO: 24, wherein the fiber yield and fiber strength were improved compared to retting with a composition which does not comprise the pectin lyase variant.

In an embodiment of the method for retting of plant fibers, the plant is flax, hemp, jute, and coir from coconut.

The present invention provides a method for fermenting cocoa beans or coffee beans, said method comprising treating the cocoa beans or coffee beans with a composition comprising the pectin lyase variant of SEQ ID NO: 24.

The present invention also provides a composition for desizing cotton fibers comprising the pectin lyase variant of claim SEQ ID NO: 24.

In an embodiment of the method for desizing cotton fibers, said method further comprises an alpha-amylase, a cellulase, a hemicellulase and/or a lipase.

In an embodiment of the method for desizing cotton fibers, the alpha-amylase is any one of the above-described alpha-amylase variant of SEQ ID NO: 1, SEQ ID NO: 2 or SEQ ID NO: 17, the cellulose is the above-described cellulase variant of SEQ ID NO: 5 or SEQ ID NO: 18, and/or the lipase is the herein-described lipase variant of SEQ ID NO: 35.

The present invention also provides a method for removing non-cellulosic impurities from cotton fiber, said method comprising treating the cotton fiber with a composition comprising the pectin lyase variant of SEQ ID NO: 24.

The present invention further provides a method for pre-treating wastewater from citrus processing, said method comprising treating the wastewater with a composition comprising the pectin lyase variant of SEQ ID NO: 24, wherein said pre-treating improves removal of pectin the wastewater compare to treatment with a composition that does not comprise the pectin lyase variant.

The present invention still further provides a method for paper making, said method comprising treating solubilized polysaccharaides with a composition comprising the pectin lyase variant of SEQ ID NO: 24.

In an embodiment of the method for paper making, said method further comprises alkaline peroxide bleaching of pulp to solubilize polysaccharides before treating the solubilized polysaccharaides with a composition comprising the pectin lyase variant of SEQ ID NO: 24.

The present invention provides a method for preparing animal feed, said method comprising pre-treating animal feed with a composition comprising the pectin lyase variant of SEQ ID NO: 24, wherein said pre-treating improves digestion of the animal feed to digestion of animal feed a composition that does not comprise the pectin lyase variant.

The present invention also provides a method for extracting citrus oil from citrus peel, said method comprising treating citrus peel with a composition comprising the pectin lyase variant of SEQ ID NO: 24.

### Phytase Variants

Phytase is a class of phosphatase enzyme that hydrolyzes phytic acid to release a useable form of organic phosphorus. Phytic acid a storage form of phosphorus found in plants, seeds, such as oil seeds, cereals and grains, which is not bioavailable to nonruminants, since these animals lack the phytase enzyme. Phytases are found in plants, fungi, bacteria and ruminant animal, such as cattle and sheep. Nonruminants, such as humans, dogs, and birds, do not produce phytase. Animal feed may be supplemented with phytase to release phytate-bound nutrients, such as calcium, phosphorus, other minerals, such as zinc, iron and magnesium, carbohydrates, and proteins. Phytase may be added to grains, legumes, seeds and corn to release the above-enumerated minerals, thereby producing functional food. The phytase variants provided herein may be used as described above, and also be administered as a supplement to humans or other nonruminants to cleave phosphorus, calcium, magnesium and iron from plant-based food, seeds, and grains.

The phytase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type phytase will denature, decompose and/or under which the phytase enzymatic function is significantly reduced or eliminated.

The present invention provides a phytase variant of a wild-type phytase from Escherichia coli of SEQ ID NO: 25 comprising mutations:
a) P26G, E27D, S37M, A47P, T48P, D57H, A58P, T61Q, K65A, L66A, V89R, A90Q, A95Q, P100C, S102D, D110N, E113Q, G119A, E120A, L126A, A131C, T133R, T136Y, T140M, S141G, S142T, P143M, V154G, Q156C, A160K, N161Q, S168K, S173G, A175Q, D176A, F177E, T178H, G179E, H180R, T183A, F185Y, R190K, L201C, Q206C, L235R, T240A, P252Q, E253D, G255A, S262E, H263Q, N266A, T267R, L269M, S270E, Y277H, Q280F, T282A, E284Y, R287A, S288A, T291G, M298Q, K308N, I322M, A335M, E337G, L342F, E354A, R362D, L363K, S364A, N366Y, S367K, I370V, S373A, F376Y, K385A, N391A, T392Q, T400C, and G417Q;
b) P26G, S37M, T48P, K65A, L66A, V89R, A90Q, A95Q, P100C, S102A, D110N, E120A, L126A, A131C, T140L, S141G, S142T, P143M, V154G, Q156C, N161Q, S168K, S173G, A175Q, G179A, T183E, F185Y, R190K, L201C, Q206C, T240A, P252Q, G255A, S262A, H263Q, T267R, Y277H, T282A, E284Y, R287A, S288A, A335M, L342F, E354K, R362D, S364A, N366Y, S367K, F376Y, K385A, T392Q, T400C, and G417Q;
c) P26G, S37M, T48P, K65A, L88W, V89M, A90Q, A95Q, P100C, S102A, D110N, E120A, L126A, A131C, T140L, S141G, S142T, P143M, V154G, Q156C, S168K, S173G, A175Q, G179R, F185Y, R190K, L201C, Q206C, T240A, P252Q, G255A, S262A, H263Q, Y277H, T282A, E284Y, S288A, A335M, L342F, E354K, R362D, N366Y, K385A, T400C, and G417Q;
d) P100C, A131C, Q156C, L201C, Q206C, and T400C;
e) P26G, S37M, K65A, L88W, V89M, A95Q, P100C, S102P, D110N, E120A, L126A, A131C, T140M, S142T, P143M, V154G, Q156C, S173G, A175Q, G179R, R190K, L201C, Q206C, T240A, P252Q, S262A, H263Q, Y277H, T282A, E284Y, S288A, E354K, R362D, N366Y, T400C, and G417Q;
f) P26G, S37M, T48P, D57S, A58P, K65A, L66A, V89R, A90Q, A95Q, P100C, S102A, D110N, G119A, E120A, L126A, A131C, T140L, S141G, S142T, P143M, V154G, Q156C, A160K, N161Q, S168K, S173G, A175Q, T178H, G179E, H180R, T183A, F185Y, R190K, L201C, Q206C, T240A, P252Q, G255A, S262A, H263Q, N266A, T267R, Y277H, Q280F, T282A, E284Y, R287A, S288A, T291G, M298Q, K308N, A335M, E337G, L342F, E354A, R362D, S364A, N366Y, S367K, F376Y, K385A, N391A, T392Q, T400C, and G417Q;
g) P26G, S37M, L88W, V89M, A95Q, P100C, S102P, D110N, E120A, L126A, A131C, T140M, P143M, V154G, Q156C, S173G, A175Q, G179R, R190K, L201C, Q206C, T240A, P252Q, S262A, H263Q, Y277H, T282A, E284Y, R362D, N366Y, T400C, and G417Q;
h) P26G, S37M, T48P, K65A, L88W, V89M, A90Q, A95Q, P100C, S102A, D110N, E120A, L126A, A131C, T140L, S141G, S142K, P143M, V154G, Q156C, S168K, S173G, A175Q, G179R, F185Y, R190K, L201C, Q206C, T240A, P252Q, G255A, S262A, H263Q, Y277H, T282A, E284Y, S288A, A335M, L342F, E354K, R362D, N366Y, K385A, T400C, and G417Q;
i) P26G, E27D, S37M, A47P, T48P, L50T, D53R, V54M, D57R, A58P, T61Q, K65A, L66A, Q84F, L88M, V89R, A90Q, D91Q, A95Q, P100C, S102D, I108W, D110N, E113Q, G119A, E120A, F122Y, L126A, D129G, A131C, I132V, T133R, T140M, S141G, S142T, P143M, V154G, Q156C, A160Q, N161Q, V162A, D164A, I166V, S168K, S173G, A175Q, D176A, F177E, T178H, G179E, H180R, T183A, F185Y, E187Q, R190K, L201C, Q206C, S234A, L235R, T240A, P252Q, E253D, G255A, T260H, S262E, H263Q, N266A, T267R, S270A, Y277H, Q280F, T282A, E284Y, R287A, S288A, D295Q, L296F, M298Q, A299T, K308N, Q309N, T314A, L315H, L320G, I322M, A335M, E337G, L342F, E354A, R362D, L363K, S364A, D365A, N366Y, S367K, I370V, S373A, F376Y, M382L, D384Q, K385A, N391A, T392Q, T400C, A402Q, A416D, and G417R;
j) P26G, E27D, S37M, A47P, T48P, D57R, A58P, T61Q, K65A, L66A, V89R, A90Q, A95Q, P100C, S102D, D110N, E113Q, G119A, E120A, L126A, A131C, T133R, T136Y, T140M, S141G, S142T, P143M, V154G, Q156C, A160K, N161Q, T163V, S168K, S173G, A175Q, D176A, F177E, T178H, G179E, H180R, T183A, F185Y, R190K, L201C, Q206C, L235R, T240A, P252Q, E253D, G255A, S262E, H263Q, N266A, T267R, L269M, Y277H, Q280F, T282A, E284Y, R287A, S288A, P292Y, M298Q, K308N, I322M, A335M, E337G, L342F, E354A, R362D, L363K, S364A, N366Y, S367K, I370V, S373A, F376Y, K385A, N391A, T392Q, T400C, and G417Q;
k) P26G, E27D, S37M, A47P, T48P, L50T, D53R, V54M, D57R, A58P, T61Q, K65A, L66A, Q84F, L88M, V89R, A90Q, D91Q, A95Q, P100C, S102D, D110N, E113Q, G119A, E120A, F122Y, L126A, D129G, A131C, I132V, T133R, T140M, S141G, S142T, P143M, V154G, Q156C, A160Q, N161Q, V162A, D164A, I166V, S168K, S173G, A175Q, D176A, F177E, T178H, G179E, H180R, T183A, F185Y, E187Q, R190K, L201C, Q206C, S234A, L235R, T240A, P252Q, E253D, G255A, T260H, S262E, H263Q, N266A, T267R, S270A, Y277H, Q280F, T282A, E284Y, R287A, S288A, T291G, D295R, L296F, M298Q, A299T, K308N, Q309N, T314A, L320G, I322M, A335M, E337G, L342F, E354A, R362D, L363K, S364A, D365A, N366Y, S367K, I370V, S373A, F376Y, M382L, D384Q, K385A, N391A, T392Q, T400C, A402Q, A416D, and G417R;
l) P26G, S37M, L88W, V89M, A95Q, P100C, S102P, D110N, E120A, L126A, A131C, T140M, P143M, V154G, Q156C, S173G, A175Q, G179R, R190K, L201C, Q206C, T240A, P252Q, S262A, H263Q, Y277H, T282A, E284Y, L315H, R362D, N366Y, T400C, and G417Q;
m) P26G, S37M, T48P, D57S, A58P, K65A, L66A, V89R, A90Q, A95Q, P100C, S102A, D110N, G119A, E120A, L126A, A131C, T140M, S141G, S142K, P143M, V154G, Q156C, A160K, N161Q, S168K, S173G, A175Q, T178H, G179E, H180R, T183A, F185Y, R190K, L201C, Q206C, T240A, P252Q, G255A, S262A, H263Q, N266A, T267R, Y277H, Q280F, T282A, E284Y, R287A, S288A, T291G, M298Q, K308N, A335M, E337G, L342F, E354A, R362D, S364A, N366Y, S367K, F376Y, K385A, N391A, T392Q, T400C, and G417Q; or
n) P26G, S37M, T48P, K65A, L66A, V89R, A90Q, A95Q, P100C, S102A, D110N, E120A, L126A, A131C, T140M, S141G, S142T, P143M, V154G, Q156C, N161Q, S168K, S173G, A175Q, G179E, T183E, F185Y, R190K, L201C, Q206C, T240A, P252Q, G255A, S262E, H263Q, T267R, Y277H, T282A, E284Y, R287A, S288A, A335M, E354K, R362D, S364A, N366Y, S367K, F376Y, K385A, T392Q, T400C, G417Q.

The present invention provides a phytase variant of a wild-type phytase from Buttiauxella sp. of SEQ ID NO: 26 comprising mutations:
a) T43V, S50M, R65D, V67W, L79P, E87A, I90M, R91A, F96Y, R98H, K100W, I106L, N111G, P113C, D123N, G132A, E133Q, L139A, G144C, A156M, K164I, I167D, S169C, N173E, E182R, Q184H, P186S, L195A, T203E, Q214C, D219C, L249I, Y261Q, P266A, K267D, N272R, E278Q, T288A, A310Y, N313G, P329L, F335V, N345A, S347A, N351G, A375R, K380T, S384R, Q411R, K413N, A422P, E423D, P429S, T432S, M434L, Q437H, and G442A;
b) T43L, S50M, R65D, V67W, L79P, E87A, I90M, R91A, F96Y, R98H, K100W, I106L, Q109A, N111G, P113C, D123N, G132A, E133A, L139A, G144C, N152D, A156M, K164I, I167D, S169C, N173E, E182R, Q184H, P186S, L195A, T203E, Q214C, D219C, I234L, I247L, G248A, Y261Q, P266Q, K267D, N272R, E278Q, A281K, T288A, A310Y, N313G, P329H, F335M, I336L, N345A, S347A, N351G, A375R, S378A, K380T, S384R, Q411R, K413N, A422P, E423D, P429E, T432S, M434L, Q437H, and G442A;
c) S39P, G40D, T43V, S50M, M60S, M64L, R65Q, D66K, V67W, L79P, I82L, E87A, I90M,

R91A, V92Q, F96Y, E99R, K100W, I106L, Q109A, N111G, P113C, S117T, D123N, L129E, G132A, E133Q, L139A, G144C, N152D, A156M, K164I, I167D, S169C, N173E, I175A, E182R, Q184G, N185G, P186S, L195A, N202A, T203Q, Q214C, D219C, I234L, I247L, G248A, E260Q, Y261Q, P266Q, K267D, N272R, S275D, E278Q, A281K, K284A, T288A, A294N, I299V, P306N, L308M, A310Y, I311L, N313G, N318Q, A319P, I327D, S328A, P329Q, F335L, I336L, N345A, S347A, N351G, S353H, T355N, L374W, A375R, S378A, K380T, S384R, Q411R, K413N, A422P, E423D, P429E, T432S, M434L, Q437E, S438A, V439L, and G442A;
d) T43V, S50M, R65Q, L79P, E87A, I90M, R91A, F96Y, R98H, K100W, I106L, N111G, P113C, G132A, E133Q, L139A, G144C, A156M, K164I, I167D, S169C, N173E, E182R, P186S, L195A, Q214C, D219C, Y261Q, P266Q, K267D, E278Q, A281K, A310Y, N313G, P329H, F335M, K380T, S384R, Q411R, K413N, A422P, E423D, P429S, T432S, M434L, Q437H, and G442A;
e) T43V, S50M, R65D, V67W, L79P, E87A, R91A, F96Y, R98H, K100W, I106L, N111G, P113C, D123N, G132A, E133Q, L139A, G144C, A156M, K164I, I167D, S169C, N173E, E182R, Q184H, P186S, L195A, T203E, Q214C, D219C, L249I, Y261Q, P266Q, K267D, N272R, E278Q, T288A, A310Y, N313G, P329H, F335M, N345A, S347A, N351G, A375R, K380T, S384R, Q411R, K413N, A422P, E423D, P429S, T432S, M434L, Q437H, G442A;
f) S39P, T43V, S50M, M60S, R65D, D66K, V67W, L79P, I82L, E87A, I90M, R91A, F96Y, R98H, K100W, I106L, Q109A, N111G, P113C, D123N, G132A, E133Q, L139A, G144C, N152D, A156M, K164I, I167D, S169C, N173E, I175A, E182R, Q184G, N185G, P186S, L195A, N202D, T203E, Q214C, D219C, I234L, G248A, L249I, E260Q, Y261Q, P266Q, K267D, N272R, S275D, E278Q, T288A, A294N, A310Y, N313G, N318H, S328A, P329Q, N331A, F335M, I336L, N345A, S347A, N351G, T355N, L374W, A375R, S378A, K380T, Y382W, S384R, Q411R, K413N, A422P, E423D, P429E, T432S, M434L, Q437E, and G442A;
g) T43V, S50M, R65D, D66K, V67W, L79P, I82L, E87A, I90M, R91A, F96Y, R98H, K100W, I106L, Q109A, N111G, P113C, D123N, G132A, E133Q, L139A, G144C, N152D, A156M, K164I, I167D, S169C, N173E, E182R, Q184H, N185G, P186S, L195A, N202D, T203E, Q214C, D219C, I234L, G248A, L249I, E260Q, Y261Q, P266A, K267N, N272R, S275D, E278Q, T288A, A294N, A310Y, N313G, N318H, S328A, P329L, N331A, F335M, I336L, N345A, S347A, N351G, T355N, L374W, A375R, S378A, K380T, S384R, Q411R, K413N, A422P, E423D, P429E, T432S, M434L, Q437H, and G442A;
h) P113C, G144C, S169C, Q214C, D219C, Q411R, K413N, A422P, E423D, P429S, T432S, M434L, Q437H, and G442A;
i) T43V, S50M, R65D, D66K, V67W, L79P, I82L, E87A, I90M, R91A, F96Y, R98H, K100W, I106L, Q109A, N111G, P113C, D123N, G132A, E133Q, L139A, G144C, N152D, A156M, K164I, I167D, S169C, N173E, E182R, N185G, P186S, L195A, N202D, T203E, Q214C, D219C, I234L, G248A, L249I, E260Q, Y261Q, P266Q, K267N, N272R, S275D, E278Q, T288A, A294N, A310Y, I311L, N313G, S328A, P329L, F335M, I336L, N345A, S347A, N351G, S353H, T355N, L374W, A375R, S378A, K380T, S384R, Q411R, K413N, A422P, E423D, P429E, T432S, M434L, Q437H, and G442A;
j) T43V, S50M, R65D, L79P, E87A, I90M, R91A, F96Y, R98H, K100W, I106L, N111G, P113C, G132A, E133Q, L139A, G144C, A156M, K164I, I167D, S169C, N173E, E182R, P186S, L195A, Q214C, D219C, I247L, Y261Q, P266A, K267N, E278Q, A281K, A310Y, N313G, P329H, F335M, K380T, S384R, Q411R, K413N, A422P, E423D, P429S, T432S, M434L, Q437H, and G442A;
k) S39P, T43L, S50M, M60S, R65Q, D66K, V67W, L79P, I82L, E87A, I90M, R91A, V92A, F96Y, R98H, K100W, I106L, Q109A, N111G, P113C, D123N, L129E, G132A, E133A, L139A, G144C, N152D, A156M, K164I, I167D, S169C, N173E, I175A, E182R, Q184G, N185G, P186S, L195A, N202E, T203E, Q214C, D219C, I234L, I247L, G248A, E260Q, Y261Q, P266Q, K267D, N272R, S275D, E278Q, T288A, A294N, A310Y, I311L, N313G, A319P, I327D, S328A, P329Q, F335L, I336L, N345A, S347A, N351G, S353H, T355N, L374W, A375R, S378A, K380T, S384R, Q411R, K413N, A422P, E423D, P429E, T432S, M434L, Q437E, and G442A;
l) T43V, S50M, R65D, V67W, L79P, E87A, I90M, R91A, F96Y, R98H, K100W, I106L, Q109A, N111G, P113C, D123N, G132A, E133Q, L139A, G144C, N152D, A156M, K164I, I167D, S169C, N173E, E182R, Q184H, P186S, L195A, T203E, Q214C, D219C, I234L, I247L, G248A, Y261Q, P266Q, K267N, N272R, E278Q, A281K, T288A, A310Y, N313G, P329H, N331A, F335M, I336L, N345A, S347A, N351G, A375R, S378A, K380T, S384R, Q411R, K413N, A422P, E423D, P429E, T432S, M434L, Q437H, and G442A;
m) S39P, T43V, S50M, M60S, R65Q, D66K, V67W, L79P, I82L, E87A, I90M, R91A, V92A, F96Y, R98H, K100W, I106L, Q109A, N111G, P113C, D123N, L129E, G132A, E133Q, L139A, G144C, N152D, A156M, K164I, I167D, S169C, N173E, I175A, E182R, Q184G, N185G, P186S, L195A, N202A, T203Q, Q214C, D219C, I234L, I247L, G248A, E260Q, Y261Q, P266Q, K267N, N272R, S275D, E278Q, T288A, A294N, A310Y, I311L, N313G, A319P, I327D, S328A, P329Q, F335L, I336L, N345A, S347A, N351G, T355N, L374W, A375R, S378A, K380T, S384R, Q411R, K413N, A422P, E423D, P429E, T432S, M434L, Q437E, and G442A;
n) S39P, T43V, S50M, M60S, R65Q, D66K, V67W, L79P, I82L, E87A, I90M, R91A, F96Y, R98H, K100W, I106L, Q109A, N111G, P113C, D123N, L129E, G132A, E133Q, L139A, G144C, N152D, A156M, K164I, I167D, S169C, N173Q, I175A, E182R, Q184G, N185G, P186S, L195A, N202D, T203E, Q214C, D219C, I234L, G248A, E260Q, Y261Q, P266Q, K267N, N272R, S275D, E278Q, T288A, A294N, A310Y, I311L, N313G, S328A, P329Q, F335M, I336L, N345A, S347A, N351G, T355N, L374W, A375R, S378A, K380T, Y382W, S384R, Q411R, K413N, A422P, E423D, P429E, T432S, M434L, Q437E, and G442A;
o) S39A, G40D, T43V, S50M, M60S, M64L, R65Q, D66K, V67W, L79P, I82L, E87A, Q88H, I90M, R91A, V92Q, F96Y, R98H, E99R, K100W, I106L, Q109A, N111G, P113C, S117T, D123N, V124I, L129E, G132A, E133Q, L139A, G144C, N152D, A156M, K164I, I167D, S169C, N173E, I175A, E182R, Q184G, N185G, P186S, L195A, N202A, T203Q, Q214C, D219C, I234L, I247L, G248A, E260Q, Y261Q, P266Q, K267D, N272R, S275D, E278Q, A281K, K284A, T288A, A294N, I299V, P306N, L308M, A310Y, I311L, N313G, N318Q, I327D, S328A, P329Q, F335L, I336L, N345A, S347A, N351G, T355N, L374W, A375R, S378A, K380T, S384R, Q411R, K413N, A422P, E423D, P429E, T432S, M434L, Q437E, S438A, V439L, and G442A.

In an embodiment, the present invention provides a nucleic acid sequence encoding the above-described phytase variant of any one of SEQ ID NO: 25 or SEQ ID NO: 26.

In another embodiment of the present invention, an expression vector comprises the nucleic acid sequence encoding the phytase variant of any one of SEQ ID NO: 25 or SEQ ID NO: 26.

In an embodiment, the present invention provides a host cell transformed with the expression vector comprising the nucleic acid sequence encoding the phytase variant of any one of SEQ ID NO: 25 or SEQ ID NO: 26.

In a further embodiment, the present invention provides a method for producing a phytase variant, said method comprising expressing the phytase variant in the transformed host cell, wherein the host cell is transformed with the expression vector comprising the nucleic acid sequence encoding the phytase variant of any one of SEQ ID NO: 25 or SEQ ID NO: 26.

The present invention provides a method for releasing organic phosphorus from ingested oil seeds and grains in a subject, said method comprising administering to the subject a composition comprising the phytase variant of any one of SEQ ID NO: 25 or SEQ ID NO: 26.

In an embodiment of the method for releasing organic phosphorus from ingested oil seeds and grains in a subject, the subject is a non-ruminant.

In another embodiment of the method for releasing organic phosphorus from ingested oil seeds and grains in a non-ruminant, the non-ruminant is a human, a dogs or a bird.

The present invention also provides a method for preparing animal feed, said method comprising adding to animal feed a composition comprising the phytase variant of any one of SEQ ID NO: 25 or SEQ ID NO: 26.

In an embodiment of the method for preparing animal feed, the phytase variant of any one of SEQ ID NO: 25 or SEQ ID NO: 26 enhances nutitional value of the animal feed by releasing a phytate-bound nutrient, wherein the phytate-bound nutrient is calcium, phosphorus, carbohydrates, and/or proteins.

### Polygalacturonase Variants

Polygalacturonase, also called a pectin depolymerase, is an enzyme that hydrolyzes alpha-1, 4 glycosidic bonds between galacturonic acid residues, thereby degrading pectin. Naturally occurring polygalacturonase softens and sweetens fruit during ripening. Polygalacturonase is used in the production of fruit juices to reduce viscosity and clarify the fruit juices. The presently provided polygalacturonase variants may be used in fruit ripening, and in the extraction and clarifying of fruit juices; these polygalacturonase variants may be used together with the above-described pectin lyase variants and/or pectozyme in the production of fruit juices.

The present invention provides a polygalacturonase variant of a wild-type polygalacturonase from Aspergillus niger of SEQ ID NO: 27 comprising mutations:
a) T36C, A43E, S48Q, S51C, A71L, D73S, T78I, D115G, S122G, V140L, K146T, I148L, N168D, D179S, G187A, G195Y, I210C, S217H, S226C, K245Q, S250T, S252N, D256N, A258T, I266N, K268G, E269G, D272S, E275N, Q282I, I286C, S301G, V315F, G319N, L324V, E325A, D326S, Q330N, A356T, and D359K;
b) T36C, A43E, S51C, V140L, K146S, I148L, I210C, S217N, S226C, K245Q, A258T, I266N, K268G, E269G, D272S, Q282I, S301G, G319N, L324V, D326S, A356T, and D359K;
c) T36C, A40H, E42D, A43E, E45K, S46A, I47Q, S48Q, S51C, S69K, A71L, D73S, T78I, M106Q, D115G, S122G, D139K, V140L, F145I, K146T, I148L, I158V, V165L, N168D, D179S, G187A, I210C, I212V, S217N, S226C, K245Q, S250T, S252C, D256N, A258D, I266N, K268G, E269G, D272S, E275N, I276F, Q282I, I286C, S301G, I310V, D314N, V315F, G319N, L324V, E325A, D326S, Q330N, D343N, S352T, A356T, D358T, and D359K;
d) T36C, S51C, I210C, S226C, A356T, and D359K;
e) T36C, A43E, S48Q, S51C, A71L, T78I, S122G, V140L, K146T, I148L, G195Y, I210C, S217N, S226C, K245Q, D256N, A258D, I266N, K268G, E269G, D272S, E275N, Q282I, S301G, G319N, L324V, D326S, A356T, and D359K;
f) T36C, A43E, S48Q, S51C, A71L, D73S, T78I, D115G, S122G, V140L, K146T, I148L, N168D, D179S, G187A, G195Y, I210C, S217N, S226C, K245Q, S250T, S252N, D256N, A258D, I266N, K268G, E269G, D272S, E275N, Q282I, I286C, S301G, V315F, G319N, L324V, E325A, D326S, Q330N, A356T, and D359K;
g) T36C, A43E, S51C, V140L, K146T, I148L, I210C, S217N, S226C, K245Q, A258T, I266N, K268G, E269G, D272S, Q282I, S301G, G319N, L324V, D326S, A356T, and D359K;
h) T36C, E42D, A43E, S46A, I47Q, S48Q, S51C, A71L, D73S, T78I, D115G, S122G, V140L, K146T, I148L, N168D, D179S, G187A, G195Y, I210C, S217N, S226C, K245Q, S250T, S252C, D256N, A258T, I266N, K268G, E269G, D272S, E275N, Q282I, I286C, S301G, I310V, V315F, G319N, L324V, E325A, D326S, Q330N, D343N, S352T, A356T, and D359K;
i) T36C, A43E, S48Q, S51C, A71L, D73S, T78I, D115Q, S122G, V140L, K146T, I148L, N168D, G187A, G195Y, I210C, S217N, S226C, K245Q, D256N, A258D, I266N, K268G, E269G, D272S, E275N, Q282I, I286C, S301G, G319N, L324V, E325S, D326S, Q330N, A356T, and D359K;
j) T36C, A40H, E42D, A43E, E45K, S46A, I47Q, S48Q, S51C, S69K, A71L, D73S, T78I, M106Q, D115Q, S122G, D139K, V140L, F145I, K146T, I148L, I158V, V165L, N168D, D179S, G187A, G195Y, I210C, S217N, S226C, K245Q, S250T, S252C, S255T, D256N, A258T, I266N, K268G, E269G, D272S, E275N, I276F, Q282I, I286C, S301G, S307T, I310V, D314N, V315F, G319N, L324V, E325A, D326S, Q330N, D343N, S352T, A356T, D358T, and D359K; or
k) T36C, A43E, S48Q, S51C, A71L, D73S, T78I, S122G, V140L, K146T, I148L, N168D, G187A, G195Y, I210C, S217N, S226C, K245Q, D256N, A258D, I266N, K268G, E269G, D272S, E275N, Q282I, I286C, S301G, G319N, L324V, E325S, D326S, Q330N, A356T, and D359K.

In an embodiment, the present invention provides a nucleic acid sequence encoding the above-described polygalacturonase variant of SEQ ID NO: 27.

In another embodiment of the present invention, an expression vector comprises the nucleic acid sequence encoding the polygalacturonase variant of SEQ ID NO: 27.

In an embodiment, the present invention provides a host cell transformed with the expression vector comprising the nucleic acid sequence encoding the polygalacturonase variant of SEQ ID NO: 27.

In a further embodiment, the present invention provides a method for producing a polygalacturonase variant, said method comprising expressing the polygalacturonase variant in the transformed host cell, wherein the host cell is transformed with the expression vector comprising the nucleic acid sequence encoding the polygalacturonase variant of SEQ ID NO: 27.

The present invention provides a method for extracting fruit juice from a fruit, said method comprising treating the fruit with the polygalacturonase variant of SEQ ID NO: 27.

In an embodiment of the method for extracting fruit juice from a fruit, said method further comprises treating the fruit with a pectin lyase.

In another an embodiment of the method for extracting fruit juice from a fruit, the pectin lyase is an above-described pectin lyase variant of SEQ ID NO: 24.

The present invention also provides a method for clarifying fruit juice, said method comprising treating the fruit juice with the polygalacturonase variant of SEQ ID NO: 27.

In an embodiment of the method for clarifying fruit juice, said method further comprises treating the fruit juice with a pectin lyase.

In another embodiment of the method for extracting fruit juice from a fruit, the pectin lyase is an above-described pectin lyase variant of SEQ ID NO: 24.

### Sulfhydryl Oxidase Variants

Sulfhydryl oxidase, also called thiol oxidase or SOX, is an enzyme which oxidizes free sulfhydryl groups in proteins and in thiol-containing small thiol molecules to form an organic disulfide (R—S—S—R). Disulfide bonds, S—S bonds, have an important role in the folding and stability of some proteins, such as proteins secreted to the extracellular medium and transmembrane proteins. Thus, disulfide bond formation in proteins and small molecules affects the functionality of the proteins and small molecules. In proteins, a disulfide bond, also called a cystine, is formed between the sulfur atoms (thiol groups) of two cysteine amino acids to connect polypeptide chains; the connected chains form a part of the primary structure of a protein. Disulfide bonds occur either between polypeptide chains (intermolecular disulfide bonds) when the cysteines are part of the same polypeptide chain, contributing to the tertiary structure of a protein, or between different polypeptide chains (intramolecular disulfide bonds), contributing to the quaternary structure of a protein.

Sulfhydryl oxidase is produced in prokaryotes, plants, mammals, including humans, bacteria, such as Bacillus subtilis, fungi, such as Aspergillus, Mycothecium, Piricularia and Polyporus, and certain viruses. In the baking industry, sulfhydryl oxidase is an additive to optimize dough volume, strength, and consistency, including the stability and rheological properties of the dough, pastries and baked products without adversely affecting the taste and aroma. In the food industry, sulfhydryl oxidases are used in food processing, to oxidize the free sulfhydryl groups in proteins and thiol-containing small molecules and form novel covalent bonds between specific residues in proteins and/or peptides, thus forming or modifying the protein network in meat, fish, imitation crabmeat, processed meat or processed fish. Sulfhydryl oxidase improves the stability properties and flavor of food products, for example, it removes the cooked or burnt-off flavor of milk after ultrahigh-temperature treatment. The oxidation of small and possibly volatile thiol molecules in food and beverages is applied to control the aroma/smell of food products, including by not limited to coffee, tea, cacao, chocolate and cheese. Sulfhydryl oxidase also is used to control microbial contamination in beverage, including wine and milk.

The sulfhydryl oxidase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type sulfhydryl oxidase will denature, decompose and/or under which the sulfhydryl oxidase enzymatic function is significantly reduced or eliminated.

The present invention provides a sulfhydryl oxidase variant of a wild-type sulfhydryl oxidase from Saccharomyces cerevisiae of SEQ ID NO: 28 comprising mutations:
a) D75E, K76T, K79E, V81L, L92Y, E99N, H109K, G113H, Y115F, E117K, S125A, Y126K, V129Q, E133K, T143E, Y159R, and D163P;
b) D75E, K76T, K79E, V81L, L92M, H109K, G113H, S125A, Y126E, V129Q, and D163P;
c) D75E, K76T, V77T, K79E, V81L, L92M, E99N, H109K, T110Q, G113E, Y115F, E117K, S125A, Y126K, V129Q, E133K, T143E, M147Q, Y159R, K161G, and D163P;
d) D75E, K76T, K79E, V81L, L92M, H109K, G113H, S125A, Y126K, V129Q, and D163P;
e) D75E, H109K, G113R, S125A, Y126K, V129Q, and D163P;
f) H109K, G113R, S125A, Y126E, V129Q, and D163P;
g) H109K, G113R, S125A, Y126K, V129Q, and D163P;
h) D74N, D75E, K76T, V77T, K79E, V81L, Y88F, L92M, E99N, H109K, T110Q, G113H, Y115F, E117K, S125A, Y126K, V129Q, E133K, V137P, T143E, M147Q, Y159R, K161G, and D163P;
i) D74N, D75E, K76T, V77T, K79E, V81L, Y88F, L92Y, E99N, R105Q, H109R, T110N, G113H, Y115F, E117R, S125A, Y126K, V129Q, E133K, V137P, T143E, M147Q, Y159R, K161G, D163P, and A168S;
j) D75E, K76T, K79E, V81L, L92Y, E99N, H109K, G113N, Y115F, E117K, S125A, Y126E, V129Q, E133K, T143E, Y159R, and D163P;
k) D74N, D75E, K76T, V77T, K79E, V81L, Y88F, L92Y, E99N, H109K, T110Q, G113E, Y115F, E117K, S125A, Y126K, V129Q, E133K, V137P, T143E, M147Q, Y159R, K161G, and D163P;
l) D74N, D75E, K76T, V77T, K79E, V81L, Y88F, L92Y, E99N, R105Q, E106K, H109K, T110Q, G113E, Y115F, E117K, S125A, Y126K, V129Q, E133K, V137P, T143E, M147Q, Y159R, K161G, D163P, and A168S; or
m) D75E, K76T, V77T, K79E, V81L, L92M, E99N, H109K, T110Q, G113N, Y115F, E117K, S125A, Y126K, V129Q, E133K, T143E, M147Q, Y159R, K161G, and D163P.

In an embodiment, the present invention provides a nucleic acid sequence encoding the above-described sulfhydryl oxidase variant of SEQ ID NO: 28.

In another embodiment of the present invention, an expression vector comprises the nucleic acid sequence encoding the sulfhydryl oxidase variant of SEQ ID NO: 28.

In an embodiment, the present invention provides a host cell transformed with the expression vector comprising the nucleic acid sequence encoding the sulfhydryl oxidase variant of SEQ ID NO: 28.

In a further embodiment, the present invention provides a method for producing a sulfhydryl oxidase variant, said method comprising expressing the sulfhydryl oxidase variant in the transformed host cell, wherein the host cell is transformed with the expression vector comprising the nucleic acid sequence encoding the sulfhydryl oxidase variant of SEQ ID NO: 28.

The present invention provides a method for increasing volume and strength of dough, said method comprising treating the dough with the sulfhydryl oxidase variant of SEQ ID NO: 28, wherein the dough comprises ascorbic acid.

In an embodiment of the method for increasing volume and strength of dough, the dough is a wheat flour.

In another embodiment of any one of said methods for increasing volume and strength of dough, said methods further comprise concurrently treating the dough with a glucose oxidase, a hemicellulase, an alpha-amylase, a xylanase, and/or an asparaginase.

In a further embodiment of any one of said methods for increasing volume and strength of dough, the alpha-amylase is any one of the above-described alpha-amylase variant of SEQ ID NO: 1, SEQ ID NO: 2 or SEQ ID NO: 17, the xylanse is any one of the above-described xylanase variant of SEQ ID NO: 14 to SEQ ID NO: 16 or SEQ ID NO: 29 to SEQ ID NO: 34.

The present invention provides a method for processing protein-based food to bond proteins, said method comprising treating the protein-based food with the sulfhydryl oxidase variant of claim 208.

In an embodiment of the method for processing protein-based food to bond proteins, the protein-based food is a meat, a fish, imitation crabmeat, processed meat or processed fish.

The present invention also provides a method for controlling microbial contamination in a beverage, said method comprising treating the beverage with the sulfhydryl oxidase variant of claim 208.

In an embodiment of the method for controlling microbial contamination in a beverage, the beverage is a wine or milk

The present invention further provides a method for producing a pharmaceutical composition, said method comprising catalyzing a reaction to oxidize free sulfhydryl groups in a protein or a thiol-containing small molecule with the sulfhydryl oxidase variant of claim 208, thereby producing the pharmaceutical composition.

In an embodiment of the method for producing a pharmaceutical composition, the protein is a therapeutic protein or an antibody.

The present invention still further provides a method for modifying a flavor of a thiol-containing food or beverage, said method comprising treating the thiol-containing food or beverage with the sulfhydryl oxidase variant of claim 208.

In an embodiment of the method for modifying a flavor of a thiol-containing food or beverage, the thiol-containing beverage is a beer, wine or a dairy product.

In an embodiment of the method for modifying a flavor of a thiol-containing food or beverage, the dairy product is a cheese or milk.

### Lipase Variants

Lipase is an enzyme that catalyzes hydrolysis of fats, oils and triglycerides. Humans produce a pancreatic lipase, the main enzyme to break down dietary fats and triglycerides in digested oils to monoglycerides and two fatty acids. Humans also produce a lingual lipase, a bile salt-dependent lipase, hepatic lipase, lipoprotein lipase, as well as gastric lipase in the infant.

Lipases may be administered to humans having a lipase deficiency; lipase is a component of pancreatic enzyme replacement therapy together with amylase and protease. The lipase variants provided herein may be administered to a subject with pancreatic disorders together with the amylase variants provided herein and the protease variants provided herein. The pancreatic disorders that may be treated with a combination of the lipase variants provided herein, the amylase variants provided herein and the protease variants provided herein, are pancreatitis, exocrine pancreatic insufficiency, digestive disorders in cystic fibrosis, surgical removal of the pancreas, pancreatic cancer, malabsorption syndrome, gallbladder malfunction or surgical removal of the gallbladder.

The lipase variants provided herein can be uses in the manner that industrial lipases are uses, including in production of cheese and yogurt, in baking, in laundry detergents, and as biocatalysts to convert vegetable oil and biomass into biodiesel. The lipase variants provided herein may be administered to a subject having a lipase deficiency to aid or supporting digestion of lipids in said, wherein the subject has a high level of cholesterol and/or a high level of triglycerides.

The lipase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type lipase will denature, decompose and/or under which the lipase enzymatic function is significantly reduced or eliminated.

The present invention provides a lipase variant of a wild-type lipase from Thermomyces lanuginosus of SEQ ID NO: 35 comprising mutations:
a) F29Y, N30D, N33K, Q37H, S39A, N55K, A62N, L74I, G113A, N116D, D118L, K120T, E121D, N123S, R130K, G131A, D133R, T136Y, S137N, S138A, Q148A, H157Y, K259V, A265S, P272T, and L286V;
b) F29Y, N30D, N33K, Q37H, S39A, N55K, A62N, L74I, G113A, N116D, D118L, K120T, E121D, N123S, R130K, G131A, D133R, T136W, S137D, S138A, Q148A, H157Y, K259V, A265S, P272T, and L286V;
c) F29Y, N33K, Q37H, L74I, G113A, T136W, S137E, H157Y, and K259V;
d) F29Y, N33K, Q37H, S39A, L74I, G113A, T136Y, S137N, H157Y, and K259V;
e) E23D, F29Y, N30D, N33K, Q37H, S39A, A52P, N55K, A62N, L74I, V82L, G113A, N116D, D118L, K120T, E121D, N123S, R130K, G131A, D133R, T136Y, S137N, S138A, A143R, Q148A, H157Y, K259V, I260C, A265S, Q271G, P272T, and L286I;
f) F29Y, N30D, N33K, Q37H, S39A, N55K, A62N, L74I, G113A, K120T, N123S, R130K, D133R, T136Y, S137N, H157Y, and K259V;
g) F29Y, N33K, Q37H, S39A, N55K, L74I, G113A, N123S, D133R, T136Y, S137N, H157Y, and K259V;
h) F29Y, N30D, N33K, Q37H, S39A, N55K, A62N, L74I, G113A, E121D, N123S, R130K, D133K, T136Y, S137N, H157Y, and K259V; or
i) F29Y, N33K, Q37H, L74I, G113A, T136W, S137N, H157Y, and K259V.

In an embodiment, the present invention provides a nucleic acid sequence encoding the above-described lipase variant of SEQ ID NO: 35.

In another embodiment of the present invention, an expression vector comprises the nucleic acid sequence encoding the lipase variant of SEQ ID NO: 35.

In an embodiment, the present invention provides a host cell transformed with the expression vector comprising the nucleic acid sequence encoding the lipase variant of SEQ ID NO: 35.

In a further embodiment, the present invention provides a method for producing a lipase variant, said method comprising expressing the lipase variant in the transformed host cell, wherein the host cell is transformed with the expression vector comprising the nucleic acid sequence encoding the lipase variant of SEQ ID NO: 35.

The present invention also provides a pharmaceutical composition comprising the lipase variant of SEQ ID NO: 35.

In an embodiment, the pharmaceutical composition comprising the lipase variant of SEQ ID NO: 35, further comprises an amylase and a protease.

In another embodiment of the pharmaceutical composition comprising the lipase variant of SEQ ID NO: 35, the amylase is the alpha-amylase is any one of the above-described alpha-amylase variant of SEQ ID NO: 1, SEQ ID NO: 2 or SEQ ID NO: 17 and the protease is any one of the above-described protease variant of SEQ ID NO: 9.

The present invention also provides a method for aiding or supporting digestion of lipids in a subject having a lipase deficiency, said method comprising administering to the subject any one of the above-described the pharmaceutical compositions comprising the lipase variant of SEQ ID NO: 35.

In an embodiment of the method for aiding or supporting digestion of lipids in a subject having a lipase deficiency, the lipase deficiency is a deficiency of pancreatic lipase, a lingual lipase and/or a bile salt-dependent lipase.

In another embodiment of the method for aiding or supporting digestion of lipids in a subject having a lipase deficiency, the lipase deficiency is a deficiency of gastric lipase in an infant.

In a further embodiment of the method for aiding or supporting digestion of lipids in a subject having a lipase deficiency, the subject has celiac disease, irritable bowel syndrome or cystic fibrosis.

In yet another embodiment of the method for aiding or supporting digestion of lipids in a subject having a lipase deficiency, the subject has a gallbladder malfunction or lacks a gallbladder.

In an embodiment of the method for aiding or supporting digestion of lipids in a subject having a lipase deficiency, the subject has a high level of cholesterol and/or a high level of triglycerides.

In another embodiment of the method for aiding or supporting digestion of lipids in a subject having a lipase deficiency, the high level of cholesterol is a level of total cholesterol of greater than or equal to 240 mg/dL.

In an embodiment of the method for aiding or supporting digestion of lipids in a subject having a lipase deficiency, the high level of cholesterol is a level of low-density lipoprotein cholesterol of greater than or equal to 160 mg/dL.

In another embodiment of the method for aiding or supporting digestion of lipids in a subject having a lipase deficiency, the high level of triglycerides of greater than or equal to 200 mg/dL.

The present invention also provides a method for preparing yogurt, said method comprising treating fermenting milk with the lipase variant of SEQ ID NO: 35, thereby preparing the yogurt.

The present invention provides a method for preparing cheese, said method comprising treating fermenting milk with the lipase variant of SEQ ID NO: 35, thereby preparing the cheese.

The present invention further provides a method for degrading lipids, said method comprising treating fats and/or oils with the lipase variant of SEQ ID NO: 35.

In an embodiment of the method for degrading lipids, the fats are animal fats and the oils are vegetable oils.

In an embodiment of the method for degrading lipids, the animal fats and vegetable oils are a component of food waste.

The present invention also provides a detergent or stain-removing spotting concentrate comprising the lipase variant of SEQ ID NO: 35.

The present invention further provides a method of laundering a fabric, said method comprising washing the fabric with the detergent or stain-removing spotting concentrate comprising the lipase variant of SEQ ID NO: 35.

The present invention provides a method of baking, said method comprising adding to a flour-containing dough the lipase variant of SEQ ID NO: 35, wherein the addition of the lipase variant improves a baked food product volume, crumb structure and freshness compared to a food product baked with four to which the lipase variant has not been added.

In an embodiment of the method of baking, the flour-containing dough does not comprise fat or lipid.

In another embodiment of the method of baking, said method further comprises adding to the flour-containing dough an alpha-amylase, a xylanase, a hemicellulase, a glucose oxidase and/or an asparaginase.

In another embodiment of the method of baking, the alpha-amylase is any one of the above-described alpha-amylase variant of SEQ ID NO: 1, SEQ ID NO: 2 or SEQ ID NO: 17,

The present invention provides a method for catalyzing production of biodiesel from vegetable oil, said method comprising adding to a biomass the lipase variant of SEQ ID NO: 35.

### Glucoamylase Variants

Glucoamylase, also known as γ-amylase or glucoamylase, is an amylolytic enzyme that hydrolyzes β-D-glucose units from the non-reducing ends of starch (polysaccharide) molecules. Glucoamylase also cleaves α-1, 6 bonds at amylopectin branching points. Thus, glucoamylase produces glucose as the sole end-product from starch and related polymers. Glucoamylase has most acidic optimum pH of all amylases: it is most active at a pH of about 3. Glucoamylase breaks down starch found in vegetables, including potatoes, corn, rice and wheat.

The optimum temperature of the glucoamylase variants provided herein is about 55° C. to 60° C. and the optimum pH is 4.5-5.0 or lower, e.g., a pH of 3.

The glucoamylase variants provided herein may be administered to a subject having a deficiency of one or more of the digestive enzymes discussed herein together with other enzymes, such as the herein provided protease variants and/or the herein provided lipase variants, to aid or support digestion of starch in a subject having a deficiency of glucoamylase, protease and/or lipase. The glucoamylase variant may be administered to treat irritable bowel syndrome together with the herein provided protease variants and/or the herein provided lipase variants.

The glucoamylase variants provided herein may be used in the herein provided process of isomerizing glucose to fructose, as the glucomaylase of step (b).

The glucoamylase variants provided herein may be used in methods of treating food waste, said methods comprising treating the food waste with any one of the herein provided glucoamylase variants.

The glucoamylase variants provided herein are stable under conditions such as high temperature, e.g., over 50° C., pH extremes, e.g., high acidity at pH of 4 or lower, in the presence of salts, alkalis and/or surfactants, and other such severe conditions under which wild-type glucoamylase will denature, decompose and/or under which the glucoamylase enzymatic function is significantly reduced or eliminated.

The present invention provides a glucoamylase variant of a wild-type glucoamylase from Talaromyces islandicus of SEQ ID NO: 36 comprising mutations:
a) I66A, R98I, L102H, D113Q, E130D, A132G, L165M, E198N, G234A, A238K, S241C, D244C, P248W, Y253C, D293C, S301C, P302S, K309M, V329K, F348Y, N351T, E439M, N456M, S461N, I463V, P466A, A470E, T475S, S478G, S479T, S481A, T482A, G483T, T486I, T491S, and P492V;
b) V43I, I66A, L90A, L102N, D113Q, E130D, A132G, L165M, S178R, E198N, T231A,

G234A, A238K, S241C, W242P, D244C, P248W, Y253C, D293C, I295A, S301C, P302S, K309M, I325K, V329K, F348Y, N351T, T397S, G413E, A420Y, D433N, T438A, E439M, N456M, S461N, I463V, P466A, A470E, T475S, S479T, S481A, G483T, T486I, T491S, and P492V;
c) V43I, N46Q, Y58K, S61G, I66A, T73K, L90A, R98I, N99A, L102H, T106Q, V107T, E109Q, D113Q, T122S, E130D, A132G, D148G, L165M, D167A, E174A, Y177N, S178R, V182S, T183N, E198N, M214S, S215H, A220M, L 226V, T231R, F232L, G234A, V236L, A238K, S241C, W242P, D244C, P248W, Y253C, D293C, I295A, S301C, P302S, N307T, K309M, V310A, V318I, L321I, I325K, E327S, V329K, F348Y, S350A, N351T, D383Q, V390T, T397S, F400Y, D402N, T409W, G413D, Y414F, I415M, S416A, T424S, S427N, Y432F, D433N, K434R, S435Y, T438A, E439M, M458A, S461N, I463V, P466H, A470E, T475S, S479T, G483T, T486I, T491S, and P492V;
d) I66A, L90A, L102N, D113Q, E130D, A132G, L165M, S178R, E198N, G234A, A238K, S241C, D244C, P248W, Y253C, D293C, I295A, S301C, P302S, K309M, I325K, V329K, F348Y, N351T, T397S, E439M, N456M, S461N, I463V, P466A, A470E, T475S, S478G, S479T, S481A, T482A, G483T, T486I, T491S, and P492V;
e) I66A, L90A, L102N, D113Q, E130D, A132G, L165M, S178R, E198N, G234A, A238K, S241C, D244C, P248W, Y253C, D293C, I295S, S301C, P302S, K309M, I325K, V329K, F348Y, N351T, T397S, E439M, N456M, S461N, I463V, P466A, A470E, T475S, S478G, S479T, S481A, T482A, G483T, T486I, T491S, and P492V;
f) V43I, I66A, L90A, L102N, D113E, E130D, A132G, L165M, S178R, E198N, T231A, G234A, A238K, S241C, W242P, D244C, P248W, Y253C, D293C, I295S, S301C, P302S, K309M, I325K, V329R, F348Y, N351T, T397S, G413E, A420Y, D433N, T438A, E439M, N456M, S461N, I463V, P466A, A470E, T475S, S479T, S481A, G483T, T486I, T491S, and P492V;
g) I66A, R98I, L102H, D113Q, E130D, A132G, L165M, E198A, G234A, A238K, S241C, D244C, P248W, Y253C, D293C, S301C, P302S, K309M, V329K, F348Y, N351T, E439M, N456M, S461N, I463V, P466A, A470E, T475S, S478G, S479T, S481A, T482A, G483T, T486I, T491S, and P492V;
h) V43I, N46Q, Y58K, S61G, I66A, T73K, L90A, R98I, N99A, L102H, T106Q, V107T, E109Q, D113E, T122S, E130D, A132G, D148G, L165M, D167A, E174A, Y177N, S178R, V182S, T183N, E198N, M214S, S215H, A220M, L226V, T231R, F232L, G234A, V236L, A238K, S241C, W242P, D244C, P248W, Y253C, D293C, I295A, S301C, P302S, N307T, K309M, V310A, V318I, L321I, I325K, E327S, V329S, F348Y, S350A, N351T, D383Q, V390T, T397S, F400Y, D402N, T409W, G413D, Y414F, I415M, S416A, T424S, S427N, Y432F, D433N, K434R, S435Y, T438A, E439M, M458A, S461N, I463V, P466H, A470E, T475S, S479T, G483T, T486I, T491S, and P492V;
i) V43I, S44A, Y58K, S61G, I66A, L90A, L102N, D113Q, E130D, A132G, D148G, L165M, S178R, E198N, M214S, L226V, T231A, G234A, A238K, S241C, W242P, D244C, P248W, Y253C, D293C, I295A, S301C, P302S, K309M, I325K, E327Q, V329S, F348Y, N351T, T397S, D402N, G413D, A420Y, D433N, T438A, E439M, N456M, S461N, I463V, P466A, A470E, T475S, S479T, S481A, G483T, T486I, T491S, and P492V;
j) V43I, S44A, Y58K, S61G, I66A, T73K, L90A, L102N, D113Q, E130D, A132G, D148G, L165M, E174A, Y177N, S178R, E198N, M214S, S215H, L226V, T231A, G234A, A238K, S241C, W242P, D244C, P248W, Y253C, D293C, I295T, S301C, P302S, N307T, K309M, V310A, V318I, I325K, E327Q, V329S, F348Y, S350A, N351T, V390T, T397S, F400Y, D402N, T409W, G413E, A420Y, D433N, K434R, S435Y, T438A, E439M, N457H, M458A, S461N, I463V, P466H, A470E, T475S, S479T, G483T, T486I, T491S, and P492V;
k) I66A, L102N, D113Q, E130D, A132G, L165M, E198N, G234A, A238K, S241C, D244C, P248W, Y253C, D293C, I295A, S301C, P302S, K309M, V329K, F348Y, N351T, E439M, N456M, S461N, I463V, P466A, A470E, T475S, S478G, S479T, S481A, T482A, G483T, T486I, T491S, and P492V;
l) A38E, V43I, S44A, Y58K, S61G, I66A, T73K, L90A, L102N, D113Q, E130D, A132G, D148G, L165M, E174A, Y177N, S178R, E198A, M214S, S215H, L226V, T231A, G234A, A238K, S241C, W242P, D244C, P248W, Y253C, D293C, I295A, S301C, P302S, N307T, K309M, V310A, V318I, I325K, E327Q, V329S, F348Y, S350A, N351T, V390T, T397S, F400Y, D402N, T409W, G413D, D433N, K434R, S435Y, T438A, E439M, N457H, M458A, S461N, I463V, P466H, A470E, T475S, S479T, G483T, T486I, T491S, and P492V;
m) V43I, S44A, Y58K, S61G, I66A, L90A, L102N, D113E, E130D, A132G, D148G, L165M, S178R, E198N, M214S, L226V, T231A, G234A, A238K, S241C, W242P, D244C, P248W, Y253C, D293C, I295A, S301C, P302S, K309M, I325K, E327Q, V329K, F348Y, N351T, T397S, D402N, G413D, A420Y, D433N, T438A, E439M, N456M, S461N, I463V, P466A, A470E, T475S, S479T, S481A, G483T, T486I, T491S, and P492V; or
n) S241C, D244C, Y253C, D293C, S301C, S461N, G462S, I463V, P466A, A470E, N474S, T475S, S478G, S479T, S481A, T482A, G483T, T486I, T491S, and P492V.

In an embodiment, the present invention provides a nucleic acid sequence encoding the above-described glucoamylase variant of SEQ ID NO: 36.

In another embodiment of the present invention, an expression vector comprises the nucleic acid sequence encoding the glucoamylase variant of SEQ ID NO: 36.

In an embodiment, the present invention provides a host cell transformed with the expression vector comprising the nucleic acid sequence encoding the glucoamylase variant of SEQ ID NO: 36.

In a further embodiment, the present invention provides a method for producing a glucoamylase variant, said method comprising expressing the glucoamylase variant in the transformed host cell, wherein the host cell is transformed with the expression vector comprising the nucleic acid sequence encoding the glucoamylase variant of SEQ ID NO: 36.

The present invention provides a pharmaceutical composition comprising the glucoamylase variant of SEQ ID NO: 36.

In an embodiment, the present invention provides a method for aiding or supporting digestion of starch in a subject having a deficiency of glucoamylase, said method comprising administering to the subject a pharmaceutical composition comprising the glucoamylase variant of SEQ ID NO: 36.

In an embodiment of the method for aiding or supporting digestion of starch in a subject having a deficiency of glucoamylase, the aiding or supporting digestion of starch decreases malabsorption, bloating, abdominal discomfort and gas compared to digestion upon administration of a pharmaceutical composition that does not comprise the glucoamylase variant.

In another embodiment of the method for aiding or supporting digestion of starch in a subject having a deficiency of glucoamylase, said method further comprises administering to the subject a protease and/or a lipase.

In further embodiments of any one of the above-described methods for aiding or supporting digestion of starch in a subject having a deficiency of glucoamylase, the subject has irritable bowel syndrome.

In an embodiment of any one of the above-described methods for aiding or supporting digestion of starch in a subject having a deficiency of glucoamylase, the starch is a vegetable starch.

In another embodiment of any one of the above-described methods for aiding or supporting digestion of starch in a subject having a deficiency of glucoamylase, the vegetable starch is potato, corn, rice, wheat, tomato, peppers, eggplant, goji berry, paprika, or banana.

In an embodiment of the above-described method for isomerizing glucose to fructose, the glucomaylase of step (b) is any one of the herein provided glucoamylase variant of SEQ ID NO: 36.

In an embodiment, the present invention provides a method of treating food waste, said method comprising treating the food waste with any one of the above-described glucoamylase variant of SEQ ID NO: 36.

In another embodiment of said method of treating food waste, said treating of the food waste with any one of the above-described glucoamylase variant of SEQ ID NO: 36 produces glucose.

### Cas9 Variants

Clustered Regularly Interspaced Short Palindromic Repeats (CRISPR) are a repeating pattern of a DNA sequence. CRISPR-associated system 9, also known as Cas9, is an RNA-guided DNA endonuclease; this enzyme is used as a genetic engineering tool to induce site-directed double strand breaks in DNA. The target DNA of Cas9 must have a Protospacer Adjacent Motif (PAM), which consistis of the three-nucleotide sequence, -5′-NGG-3′. Cas9 may be used to edit the genome to insert, delete or replace DNA to correct genetic mutations which cause diseases. Cas9 also may be used to modify food to introduce immunity against a virus or bacteria. Cas9 further may be used to produce disease-resistant grain and livestock. Gene editing with Cas9 may also be used to produce biopharmaceuticals.

The Cas9 variants provided herein are stable under conditions such as high temperature, e.g., over 50° C., pH extremes, e.g., high acidity at pH of 4 or lower, in the presence of salts, alkalis and/or surfactants, and other such severe conditions under which wild-type Cas9 will denature, decompose and/or under which the Cas9 enzymatic function is significantly reduced or eliminated.

The present invention provides a Cas9 variant of a wild-type Cas9 from Streptococcus pyogenes of SEQ ID NO: 37 comprising mutations:
a) A14D, E28D, S33R, H45K, C84K, D99P, H117Y, E118S, D128E, R143I, K145Y, V147A, D151K, V185I, D186Q, I190K, A207E, S208N, S217T, R219K, R224D, N228K, Q232N, E236Q, T253Q, D276E, D280E, Q285K, S301Y, N313D, E315T, I316E, T337K, A341K, L342F, A371T, F379Y, V395K, N398D, G421Q, R428E, R429N, E431G, F433Y, D439K, I447L, E474D, E483D, G489E, A490K, Q493K, E520Q, Y521K, A542E, K550Q, A551Q, T559N, N560Y, S585K, E617H, V624I, T626W, E637R, E638Q, K641S, T642K, K650E, V651Q, M652L, Q654R, S679N, K688I, S689D, T707S, D711I, G740A, L751I, V754A, N762W, Q798K, T808E, E813D, V828T, D829G, Q830E, V842I, A844H, V846I, R863S, E877K, Q889M, L915E, S941N, V967S, Q975N, H989F, T999N, K1080Q, S1146C, S1163L, L1168V, G1169P, S1177Q, V1194I, K1195R, D1197E, K1259H, S1281V, S1296E, D1348N, and A1349G;
b) A14D, E28D, S33R, H45K, C84K, N92D, D99P, H117Y, E118S, D128E, R143I, K145Y, V147A, D151Q, L162C, M165L, V185I, D186Q, I190K, Q194D, A207E, S208N, S217T, R219K, R224D, N228K, Q232N, E236Q, T253Q, A263E, D276E, D280E, Q285K, S301Y, N313D, E315T, I316Q, Q334K, T337E, A341K, L342F, A371T, F379Y, V395K, N398D, I418V, G421Q, R428E, R429N, E431G, F433Y, D439E, I447L, S473T, E474D, E483D, G489E, A490K, Q493N, S494A, F502K, E520Q, Y521K, V534I, A542E, A551Q, L555Y, T559K, N560Y, R561K, S585K, E617H, V624I, T626W, E637R, E638Q, K641S, T642K, K650E, V651Q, M652L, Q654R, K656E, K688I, S689D, A693N, T707S, D711I, G740A, L751I, V754A, N762W, Q798K, T808E, E813D, K814R, V828T, D829G, V842I, A844H, V846I, R863S, E877K, Q889M, F901Y, L915E, S941N, V967S, Q975D, H989F, T999N, K1080S, F1084L, V1087I, S1146C, S1163L, L1168V, G1169P, S1177Q, E1193N, V1194I, K1195R, D1197E, S1234Y, K1259H, Q1260K, L1261K, S1281V, V1284H, S1296E, H1315N, D1348N, and A1349G;
c) A14D, E28D, S33A, H45K, S46Y, C84K, D99P, H117Y, E118S, D128E, R143I, K145Y, V147A, D151Q, L162C, M165L, V185I, D186Q, I190N, A207T, S208N, S217T, R219K, R224D, N228K, Q232N, E236Q, T253Q, A263E, D276E, D280E, Q285K, L295Q, S301Y, N313D, E315T, I316E, T337K, A341K, L342F, A371T, F379Y, P382K, V395K, N398D, G421Q, R428E, R429N, E431G, F433Y, D439K, I447L, S473T, E474D, E483D, G489E, A490K, Q493N, S494A, E520Q, Y521K, V534I, A542E, K550Q, A551Q, L555Y, T559K, N560Y, S585K, E617H, V624I, T626W, E637R, E638Q, K641S, T642K, K650E, V651Q, M652L, Q654R, K656E, S679N, K688I, S689D, T707S, D711I, G740A, L751I, V754A, N762W, T808E, E813D, V828T, D829G, Q830E, V842I, A844H, V846I, R863S, E877P, Q889M, F901Y, L915E, S941N, V967S, Q975D, H989F, T999N, K1080Q, S1146C, S1163L, L1168V, G1169P, S1177Q, V1194I, K1195R, D1197E, K1259H, Q1260K, S1281V, S1296E, H1315N, D1348N, and A1349G;
d) A14D, E28D, S33R, R44K, H45K, C84K, S91A, N92E, D99P, H117Y, E118S, D128E, R143I, K145Y, V147A, D151E, L162C, M165L, V185I, D186Q, I190K, Q194D, N206S, A207E, S208N, S217T, R219K, R224D, N228K, Q232N, E236Q, S249A, T253Q, S258T, A263E, D276E, D277E, D280E, Q289E, S301Y, N313D, E315T, I316N, S324A, Q334K, T337K, A341Q, L342F, A371T, F379Y, P382K, V395K, N398D, I418V, G421Q, R428E, R429N, E431G, D432K, F433Y, D439E, I447L, T470Q, S473T, E474D, E483D, G489E, A490K, Q493K, S494A, F502K, K510D, E520Q, Y521K, V534I, A542E, K550Q, A551E, D554E, L555H, T559K, N560Y, R561K, C578G, S585K, D609E, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711E, G740A, V747I, L751I, V754A, N762W, M785Y, Q798D, T808E, E813D, K814R, V828T, D829G, Q830E, V842I, A844H, V846I, K859R, R863S, D865K, E877K, Q889M, N892D, F901Y, L915E, S941E, D951G, L953R, V967S, Q975N, H989F, T999N, K1080Q, S1092N, T1142N, S1146C, S1163L, L1168V, G1169P, S1177K, E1193N, V1194I, K1195R, D1197E, S1234Q, K1259N, Q1260K, L1261K, S1281V, V1284H, D1292E, S1296Q, H1315N, D1348N, and A1349G;
e) A14D, E28D, S33R, H45K, C84K, N92D, D99P, H117Y, E118S, D128E, R143I, K145Y, V147A, D151Q, L162C, M165L, V185I, D186Q, I190K, Q194D, A207E, S208N, S217T, R219K, R224D, N228K, Q232N, E236Q, T253Q, A263E, D276E, D280E, Q285K, L295Q, S301Y, N313D, E315T, I316Q, Q334K, T337K, A341K, L342F, A371T, F379Y, P382K, V395K, N398D, I418V, G421Q, R428E, R429N, E431G, F433Y, D439E, I447L, S473T, E474D, E483D, G489E, A490K, Q493K, S494A, F502K, E520Q, Y521K, V534I, A542E, K550Q, A551Q, L555Y, T559K, N560Y, R561K, S585K, E617H, V624I, T626W, E637R, E638Q, K641S, T642K, K650E, V651Q, M652L, Q654R, K656E, S679N, K688I, S689D, A693N, T707S, D711I, G740A, L751I, V754A, N762W, Q798K, T808E, E813D, K814R, V828T, D829G, Q830E, V842I, A844H, V846I, R863S, E877K, Q889M, F901Y, L915E, S941N, V967S, Q975D, H989F, T999N, K1080S, F1084L, V1087I, S1146C, S1163L, L1168V, G1169P, S1177Q, E1193N, V1194I, K1195R, D1197E, S1234Y, K1259H, Q1260K, L1261K, S1281V, V1284H, S1296E, H1315N, D1348N, A1349G;
f) A14D, E28D, S33R, H45K, C84K, D99P, H117Y, E118S, D128E, R143I, K145Y, V147A, D151Q, L162V, M165L, V185I, D186Q, I190N, A207E, S208N, S217T, R219K, R224D, N228K, Q232N, E236Q, N244Q, T253Q, N259T, A263E, D276E, D280E, Q285K, S301Y, N313D, E315T, I316E, T337K, L342F, A371T, F379Y, V395E, N398D, R399K, G421Q, R428E, R429N, E431G, F433Y, D439K, I447L, S473T, E474D, E483D, G489E, A490K, Q493K, S494A, E520Q, Y521K, V534I, A542E, K550Q, A551E, L555Y, T559K, N560Y, S585K, E617H, V624I, T626W, E637R, E638Q, K641S, T642K, K650E, V651Q, M652L, Q654R, K656E, S679N, K688I, S689D, T707S, D711I, G740A, L751I, V754A, N762W, Q798K, T808E, E813D, V828T, D829G, Q830E, V842I, A844H, V846I, R863S, E877K, Q889M, F901Y, L915E, S941N, V967S, Q975D, H989F, T999N, K1080Q, S1146C, S1163L, L1168V, G1169P, S1177Q, V1194I, K1195R, D1197E, K1259H, Q1260K, S1281V, S1296E, H1315N, D1348N, A1349G;
g) K8R, A14D, E28D, S33R, H45K, C84K, S91A, N92D, D99P, S100N, K116R, H117G, E118S, D128E, R143I, K145Y, V147A, D151Q, L162I, M165L, V185I, D186Q, I190K, Q194D, A207E, S208N, S217T, R219K, R224D, N228K, Q232N, E236Q, T253Q, A263E, D276E, D280E, Q285K, S301Y, N313D, E315T, I316E, Q334K, T337K, A341Q, L342F, A371T, F379Y, P382K, V395E, N398D, R399K, I418V, G421Q, R428E, R429N, E431G, D432K, F433Y, D439E, I447L, S473T, E474D, E483D, G489E, A490K, Q493K, S494A, F502K, E520Q, Y521K, V534I, A542E, K550Q, A551Q, T559K, N560Y, R561K, S585K, E617H, V624I, T626W, E637R, E638Q, K641S, T642K, K650E, V651Q, M652L, Q654R, K656E, R668K, K688I, S689D, A693I, T707S, D711E, G740A, L751I, V754A, H758Q, N762S, Q798K, T808E, E813D, K814R, V828T, D829G, Q830E, V842I, A844H, V846I, R863S, D865K, E877K, Q889M, F901Y, L915E, S941E, L953R, Q975D, H989F, T999N, S1146C, S1163L, L1168V, G1169P, S1177K, E1193N, V1194I, K1195R, D1197E, S1234Q, N1238K, K1259H, Q1260K, L1261K, S1281V, V1284H, S1296E, H1315N, D1348N, and A1349G;
h) K8R, A14D, E28D, S33R, R44K, H45K, C84K, S91A, N92E, D99P, S100N, K116R, H117G, E118S, D128E, R143I, K145Y, V147A, D151E, L162C, M165L, V185I, D186Q, I190K, Q194D, N206S, A207E, S208N, S217T, R219K, R224D, N228K, Q232N, E236Q, S249A, T253Q, S258T, A263E, D276E, D280E, Q285K, S301Y, N313D, E315T, I316N, S324A, Q334K, T337K, A341Q, L342F, Q346Y, A371T, F379Y, P382K, V395K, N398D, I418V, G421Q, R428E, R429N, E431G, D432K, F433Y, D439E, I447L, T470Q, S473T, E474D, E483D, G489E, A490K, Q493K, S494A, F502K, E520Q, Y521K, V534I, A542E, K550Q, A551Q, L555Y, T559K, N560Y, R561K, K574R, S585K, E617H, V624I, T626W, E637R, E638Q, R639Q, K641S, T642K, H645D, D649E, K650E, V651Q, M652L, Q654R, K656E, R668K, S679N, K688I, S689D, A693N, T707S, D711I, G740A, V747I, L751I, V754A, N762W, M785Y, Q798D, T808E, E813D, K814R, V828T, D829G, V842I, A844H, V846I, K859R, R863S, D865K, E877K, Q889M, N892D, F901Y, L915E, S941E, D951G, Q975D, H989F, T999N, K1080Q, S1092N, T1142N, S1146C, S1163P, L1168V, G1169P, S1177K, E1193N, V1194I, K1195R, D1197E, S1234Q, N1238K, K1259N, Q1260K, L1261K, S1281V, V1284H, S1296E, H1315N, D1348N, and A1349G;
i) K8R, A14D, E28N, S33R, R44K, H45K, C84K, S91A, N92D, D99P, S100N, H117Y, E118S, D128E, R143I, K145Y, V147A, D151E, L162V, M165L, V185I, D186Q, I190N, Q194K, N206S, A207E, S208N, S217T, R219K, R224D, N228K, Q232N, E236Q, S249A, T253Q, S258T, A263E, D276E, D280E, Q285K, S301Y, N313D, E315T, I316N, S324A, Q334K, T337K, A341Q, L342F, Q346Y, A371T, F379Y, P382K, V395K, N398D, I418V, G421Q, R428E, R429N, E431G, D432K, F433Y, D439E, I447L, S473T, E474D, E483D, G489E, A490K, Q493K, S494A, F502K, E520Q, Y521K, V534I, A542E, K550Q, A551Q, D554E, L555Y, T559K, N560Y, R561K, S585K, E617H, V624I, T626W, E637R, E638Q, R639Q, K641S, T642K, H645D, D649E, K650E, V651Q, M652L, Q654R, K656A, R668K, K688I, S689D, A693N, T707S, D711I, G740A, V747I, L751I, V754A, H758Q, N762W, M785Y, Q798D, T808E, E813D, K814R, V828T, D829G, Q830E, V842I, A844H, V846I, K859R, R863S, D865K, E877K, Q889M, N892D, F901Y, L915E, S941E, D951G, L953R, V967S, Q975D, H989F, T999N, K1080Q, V1087I, S1092N, T1142N, S1146C, S1163P, L1168V, G1169P, S1177K, E1193N, V1194I, K1195R, D1197E, S1234Q, K1259H, Q1260K, L1261K, S1281V, V1284H, S1296E, H1315N, D1348N, and A1349G;
j) A14D, D27E, E28N, S33R, R44K, H45K, C84K, S91A, N92E, D99P, S100N, K116R, H117G, E118S, D128E, A131E, K135Q, R143I, K145Y, V147A, D151E, L162V, M165L, V185I, D186Q, I19ON, Q198N, N206S, A207T, S208N, L216F, S217T, R219K, R224D, N228K, Q232N, E236Q, S249A, L252N, T253Q, S258K, A263E, D276E, D277E, D280E, Q285K, Q289E, L295N, S301Y, N313D, E315T, I316N, S324A, E331D, Q334K, T337K, A341Q, L342F, Q346Y, A371T, S372T, E375D, F379Y, E392Y, V395K, N398D, I418V, G421Q, R428E, R429N, E431G, D432K, F433Y, D439E, I447L, S473T, E474D, E483D, G489E, A490K, Q493K, S494A, F502K, K510D, E520Q, Y521K, V534I, A542E, K550Q, A551Q, L555H, T559K, N560Y, R561K, C578G, F579Y, S585K, D609E, N613D, E615K, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711I, Q713E, G740A, V747I, L751I, V754A, N762W, M785Y, Q798N, E806D, T808Q, E813D, K814R, V828T, D829G, Q830E, V842I, A844H, V846I, K859R, R863S, S864R, D865K, E877K, Q889M, N892E, F901Y, S913T, L915E, S941E, D951G, L953R, K965N, V967S, Q975N, H989F, T999N, K1080S, F1084L, V1087I, S1092N, N1119D, T1142N, S1146C, S1163L, L1168V, G1169P, S1177K, E1193N, V1194I, K1195R, D1197E, S1234Q, V1237I, L1242N, K1259N, Q1260K, L1261K, F1262Y, Q1265E, E1275N, S1281V, R1283K, V1284H, L1286G, D1288K, D1292E, L1295E, S1296K, H1315N, D1348N, and A1349G;
k) A14D, E28D, S33R, H45K, C84K, S91A, N92E, D99P, S100N, K116R, H117G, E118S, D128E, R143I, K145Y, V147A, D151Q, L162C, M165L, V185I, D186Q, I190K, Q194D, A207E, S208N, S217T, R219K, R224D, N228K, Q232N, E236Q, N244Q, T253Q, N259T, A263E, D276E, D280E, Q285K, S301Y, N313D, E315T, I316Q, Q334K, T337K, A341E, L342F, A371T, F379Y, P382K, V395E, N398D, R399K, I418V, G421Q, R428E, R429N, E431G, D432K, F433Y, D439E, I447L, S473T, E474D, G489E, A490K, Q493K, S494A, F502K, E520Q, Y521K, A542E, K550Q, A551Q, L555Y, T559K, N560Y, R561K, S585K, E617H, V624I, T626W, E637R, E638Q, K641S, T642K, K650E, V651Q, M652L, Q654R, K656E, R668K, S679N, K688I, S689D, A693I, T707S, D711E, G740A, L751I, V754A, H758Q, N762S, Q798K, T808E, E813D, K814R, V828T, D829G, V842I, A844H, V846I, R863S, D865K, E877K, Q889M, F901Y, L915E, S941E, L953R, V967S, Q975N, H989F, T999N, S1146C, S1163L, L1168V, G1169P, S1177K, E1193N, V1194I, K1195R, D1197E, S1234Q, K1259N, Q1260K, L1261K, S1281V, V1284H, S1296E, H1315N, D1348N, and A1349G;
l) K8R, A14D, E28D, S33R, H45K, C84K, D99P, H117Y, E118S, D128E, R143I, K145Y, V147A, D151K, L162I, V185I, D186Q, I190N, A207T, S208N, S217T, R219K, R224D, N228K, Q232N, E236Q, T253Q, D276E, D280E, Q285K, S301Y, N313D, E315T, I316Q, T337K, A341K, L342F, A371T, F379Y, V395K, N398D, G421Q, R428E, R429N, E431G, F433Y, D439K, I447L, S473T, E474D, E483D, G489E, A490K, Q493K, E520Q, Y521K, V534I, A542E, K550Q, A551Q, L555Y, T559N, N560Y, S585K, E617H, V624I, T626W, E637R, E638Q, K641S, T642K, K650E, V651Q, M652L, Q654R, S679N, K688I, S689D, T707S, D711I, G740A, L751I, V754A, N762W, Q798K, T808E, E813D, V828T, D829G, V842I, A844H, V846I, R863S, E877K, Q889M, F901Y, L915E, S941N, V967S, Q975D, H989F, T999N, K1080Q, S1146C, S1163L, L1168V, G1169P, S1177Q, V1194I, K1195R, D1197E, K1259H, S1281V, S1296E, D1348N, and A1349G;
m) A14D, E28N, S33R, R44K, H45K, C84K, S91A, N92E, D99P, H117Y, E118S, D128E, K135Q, R143I, K145Y, V147A, D151E, L162V, M165L, V185I, D186Q, I190K, Q194D, Q198N, N206S, A207E, S208N, L216F, S217T, R219K, R224D, N228K, Q232N, E236Q, S249A, L252N, T253Q, S258K, A263E, D276E, D277E, D280E, Q289E, L295N, S301Y, D308G, N313D, E315T, I316N, S324A, E331D, Q334K, T337K, A341Q, L342F, Q346Y, A371T, S372T, E375D, F379Y, P382K, E392Y, V395K, N398D, I418V, G421Q, R428E, R429N, E431G, D432K, F433Y, D439E, I447L, E474D, E483D, G489E, A490K, Q493K, S494A, F502K, K510D, E520Q, Y521K, V534I, A542E, K550Q, A551Q, L555H, T559K, N560Y, R561K, C578G, F579Y, S585K, D609E, N613D, E615K, E617H, V624I, T626W, E637R, E638Q, R639Q, K641S, T642K, A644S, H645D, D649E, K650E, V651Q, M652L, Q654R, K656A, R668K, S679N, K688I, S689D, A693N, T707S, D711I, Q713E, G740A, V747I, L751I, V754A, H758Q, N762W, M785Y, Q798N, E806D, T808Q, E813D, K814R, V828T, D829G, V842I, A844H, V846I, K859R, R863S, S864R, D865K, E877K, Q889M, N892E, F901Y, S913T, L915E, S941E, D951G, L953R, K965N, V967S, Q975D, H989F, T999N, F1084L, V1087I, S1092N, N1119D, T1142N, S1146C, S1163L, L1168V, G1169P, S1177K, E1193N, V1194I, K1195Q, D1197N, S1234Q, V1237I, N1238K, K1259H, Q1260K, L1261K, F1262Y, Q1265E, E1275N, S1281V, R1283K, V1284H, L1286G, D1292K, S1296Q, H1315N, D1348N, and A1349G; or
n) A14D, E28D, S33R, R44K, H45K, C84K, S91A, N92E, D99P, S100N, H117Y, E118S, D128E, R143I, K145Y, V147A, D151E, L162V, M165L, V185I, D186Q, I190K, Q194D, N206S, A207E, S208N, S217T, R219K, R224D, N228E, Q232N, E236Q, S249A, T253Q, S258T, A263E, D276E, D277E, D280E, Q285K, Q289E, S301Y, D308G, N313D, E315T, I316N, S324A, Q334K, T337K, A341Q, L342F, Q346Y, A371T, F379Y, V395K, N398D, I418V, G421Q, R428E, R429N, E431G, D432K, F433Y, D439E, I447L, T470Q, S473T, E474D, E483D, G489E, A490K, Q493K, S494A, F502K, K510D, E520Q, Y521K, V534I, A542E, K550Q, A551Q, D554E, L555H, T559K, N560Y, R561K, C578G, S585K, D609E, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711I, G740A, V747I, L751I, V754A, H758Q, N762W, M785Y, Q798D, T808E, E813D, K814R, V828T, D829G, Q830E, V842I, A844H, V846I, K859R, R863S, D865K, E877K, Q889M, N892E, F901Y, L915E, S941E, D951G, V967S, Q975N, H989F, T999N, K1080Q, V1087I, S1092N, T1142N, S1146C, S1163L, L1168V, G1169P, S1177K, E1193N, V1194I, K1195R, D1197E, S1234Q, L1242N, K1259N, Q1260K, L1261K, S1281V, V1284H, D1292E, S1296Q, H1315N, D1348N, and A1349G.

The present invention also provides a Cas9 variant of a wild-type Cas9 from Campylobacter jejuni of SEQ ID NO: 38 comprising mutations:
a) A6G, T309I, F398L, V404L, and T405I;
b) A6G, D117E, K155L, A157S, T303K, T309I, I366E, S384K, F398L, V404I, T405V, and N477H;
c) A6G, T309I, I366E, F398L, V404I, T405V, and N477H;
d) A6G, T309I, F398L, V404I, T405I, and N477H;
e) A6G, D117E, T309I, I366E, S384K, F398L, V404I, T405V, and N477H;
f) A6G, D117E, K155L, T309I, I366E, S384K, F398L, V404I, T405V, and N477H;
g) A2K, A6G, D117E, K155L, A157S, S228E, K288D, A293K, T303K, T309I, I366D, K367E, S384K, F398L, L403K, V404I, T405V, L409E, E410Q, and N477H, or
h) A6G, D117E, K155L, T309I, I366E, S384K, F398L, V404I, T405I, and N477H.

In an embodiment, the present invention provides a nucleic acid sequence encoding the above-described Cas9 variant of any one of SEQ ID NO: 37 or SEQ ID NO: 38.

In another embodiment of the present invention, an expression vector comprises the nucleic acid sequence encoding the Cas9 variant of any one of SEQ ID NO: 37 or SEQ ID NO: 38.

In an embodiment, the present invention provides a host cell transformed with the expression vector comprising the nucleic acid sequence encoding the Cas9 variant of any one of SEQ ID NO: 37 or SEQ ID NO: 38.

In a further embodiment, the present invention provides a method for producing a Cas9 variant, said method comprising expressing the Cas9 variant in the transformed host cell, wherein the host cell is transformed with the expression vector comprising the nucleic acid sequence encoding the Cas9 variant of any one of SEQ ID NO: 37 or SEQ ID NO: 38.

The present invention provides a method for genome editing, said method comprising:
a) designing a guide RNA (gRNA) targeting sequence complementary to a specific DNA target in a double stranded DNA, wherein the specific DNA target is located in close proximity to a target gene to be edited and is located upstream of a Protospacer Adjacent Motif (PAM) sequence, wherein the PAM sequence is a binding site for Cas9;
b) coupling the gRNA to a Cas9 variant of any one of SEQ ID NO: 37 or SEQ ID NO: 38 to form a gRNA-Cas9 variant complex;
c) delivering to a target cell the gRNA-Cas9 variant complex in combination with exogenous donor DNA, wherein the gRNA guides the Cas9 variant to the specific DNA target, wherein (i) PAM sequence recognition by the Cas9 variant and binding therewith identifies the specific DNA target and (2) base pairing between the gRNA and the specific DNA target activate a Cas9 nuclease site on each strand of the double-stranded DNA to blunt-end cleave the double-stranded DNA at each nuclease site forming double stranded breaks, said breaks located 3-4 nucleotides downstream from the PAM sequence,
   wherein the exogenous donor DNA encodes a desired protein and is flanked by nucleotide sequences identical to nucleotide sequences framing the specific DNA target;
   wherein generation of the blunt-end double-stranded cleavage in the specific DNA target activates an endogenous DNA repair pathway; and
   wherein the Cas9 variant of any one of SEQ ID NO: 37 or SEQ ID NO: 38 has a binding and cleavage specificity that is increased over the binding and cleavage specificity of a Cas9 which does not comprise the mutations of the Cas9 variant of any one of SEQ ID NO: 37 or SEQ ID NO: 38.

In an embodiment of said method for genome editing, the endogenous DNA repair pathway is homologous recombination (HR).

In another embodiment of said method for genome editing, the HR results in a knock-in of the donor DNA at the specific DNA target site.

In a further embodiment of said method for genome editing, the endogenous DNA repair pathway is non-homologous end-joining (NHEJ), wherein said NHEJ either inserts or deletes nucleotides at the specific DNA target site, resulting in a knock-out of the target gene.

In an embodiment of said method for genome editing, the Cas9 variant of any one of SEQ ID NO: 37 or SEQ ID NO: 38cleaves only a single strand of the double-stranded DNA.

In another embodiment of said a method for genome editing, the target cell is cultured in vitro.

In a further embodiment of said method for genome editing, the gRNA-Cas9 variant complex is delivered by transient transfection, cytoplasmic injection or nuclear injection.

In a still further embodiment of said method for genome editing, the gRNA and the Cas9 variant of any one of SEQ ID NO: 37 or SEQ ID NO: 38 are delivered by (i) transient transfection of an expression vector comprising a gene encoding the variant Cas9 variant of any one of SEQ ID NO: 37 or SEQ ID NO: 38 and a gene encoding the gRNA; (ii) cytoplasmic injection or nuclear injection of said expression vector or of mRNA encoding the variant Cas9 variant of any one of SEQ ID NO: 37 or SEQ ID NO: 38 and of mRNA of the gRNA, or (iii) transduction of the target cell with a virus (a) expressing a gene encoding the variant Cas9 variant of any one of SEQ ID NO: 37 or SEQ ID NO: 38 and (b) expressing a gene encoding the gRNA.

The present invention provides a method for treating a subject having a deficiency of an enzyme, said method comprising administering to the subject target cells expressing a gene edited by the above-described methods for genome editing, wherein edited gene comprises a donor DNA encoding the enzyme.

The present invention also provides a method for modifying a food, said method comprising treating the food with bacteria expressing a gene edited by any one of the above-described methods for genome editing, wherein said edited gene comprises a donor DNA encoding a gene for immunity against a virus.

In an embodiment of said method for modifying a food, the food is yogurt or cheese.

The present invention also provides a method for producing a disease-resistant grain, said method comprising modifying the DNA of the grain to encode a gene edited by any one of the above-described methods for genome editing, wherein the edited gene comprises a donor DNA encoding a gene for immunity against the disease.

The present invention provides a method for producing a disease-resistant livestock, said method comprising modifying the DNA of the livestock to encode a gene edited by any one of the above-described methods for genome editing, wherein the edited gene comprises a donor DNA encoding a gene for immunity against the disease.

The present invention also provides a method for producing a biopharmaceutical, said method comprising modifying the DNA of a bacterium to encode a gene edited by any one of the above-described methods for genome editing, wherein the edited gene comprises a donor DNA encoding a gene for a biopharmaceutical.

### EXAMPLES

The list of enzymes, their host organism and template PDB that were used in the PROSS algorithm is detailed in Table 1.

TABLE 1
List of enzymes, their host organism and template
PDB that was used in the algorithm

| Enzyme | | | Template |
|---|---|---|---|
| Number | Enzyme Type | Organism | PDB |
| 1 | alpha amylase | Bacillus amyloliquefaciens | 1BAG |
| 2 | alpha amylase | Bacillus licheniformis | 1BAG |
| 3 | beta amylase | Barley | 2XFF |
| 4 | beta amylase | Sweet potato | 1FA2 |
| 5 | Cellulase | Trichoderma reesei | 1EG1 |
| 6 | Cyclodextrin | Bacillus macerans | 4JCL |
| | glucanotransferase | | |
| 7 | Cyclodextrin | Geobacillus | 1CYG |
| | glucanotransferase | stearothermophilus | |
| 8 | Glutaminase | Bacillus amyloliquefaciens | 3AGF |
| 9 | Protease | Bacillus amyloliquefaciens | 4HE6 |
| 10 | Pullulanase | Bacillus sp. | 2E8Y |
| 11 | Pullulanase | Klebsiella pneumoniae | 2FHB |
| 12 | Transglutaminase | Streptomyces mobaraensis | 3IU0 |
| 13 | Xaa-Pro dipeptidase | Alteromonas sp. | 3L24 |
| 14 | Xylanase | Aspergillus niger | 4XUY |
| 15 | Xylanase | Bacillus amyloliquefaciens | 1XXN |
| 16 | Xylanase | Penicillium | 4XUY |
| 17 | alpha amylase | Thermoactinomyces | 1BVZ |
| 18 | Cellulase | Trichoderma reesei | 1EG1 |
| 19 | Glucanase | Bacillus megaterium | 3PZT |
| 20 | Glucose isomerase | Streptomyces marinus | 1DXI |
| 21 | Inulase | Aspergillus niger | 3SC7 |
| 22 | Lactase | Kluyveromyces | 3OB8 |
| 23 | Lysozyme | Chicken | 3B6L |
| 24 | Pectin lyase | Aspergillus niger | 1QCX |
| 25 | Phytase | Escherichia coli | 1DKL |
| 26 | Phytase | Buttiauxella sp. | 4ARS |
| 27 | Polygalacturonase | Aspergillus niger | 1NHC |
| 28 | Sulfhydryl oxidase | Saccharomyces cerevisiae | 1JR8 |
| 29 | Xylanase | Talaromyces pinophilus | 1XYN |
| 30 | Xylanase | Thermomyces lanuginosus | 1YNA |
| 31 | Xylanase | Bacillus paralicheniformis | 3GTN |
| 32 | Xylanase | Actinomadura | 3ZSE |
| 33 | Xylanase | Aspergillus niger | 4XUY |
| 34 | Xylanase | Trichoderma reesi | 1XYN |
| 35 | Lipase | Thermomyces lanuginosus | 1DT3 |
| 36 | Glucoamylase | Talaromyces islandicus | 3EQA |
| 37 | Cas9 | Streptococcus pyogenes | 5FW2 |
| 38 | Cas9 | Campylobacter jejuni | 5X2G |

The wild-type amino acid sequences for each of the enzymes listed in Table 1 are enumerated in Table 2.

TABLE 2
Wild-type amino acid sequences of the above-listed enzymes,
according to the stated indices.

| Enzyme | |
|---|---|
| Number | Amino Acid Sequence |
| 1 | MFEKRFKTSLLPLFAGFLLLFHLVLSGPAAANAETANKSNEVAASSVKNGTILHAWNWSFNTLT |
| | QNMKEIRDAGYAAIQTSPINQVKEGNQGDKSMRNWYWLYQPTSYQIGNRYLGTEQEFKDMCA |
| | AAEKYGVKVIVDAVINHTTSDYAAISDEIKRIPNWTHGNTQIKNWSDRWDVTQNSLLGLYDWNT |
| | QNTEVQTYLKRFLERALNDGADGFRYDAAKHIELPDDGNYGSRFWPNITNTSAEFQYGEILQD |
| | SASRDTAYANYMNVTASNYGHSIRSALKNRNLSVSNISHYASDVSADKLVTWVESHDTYANDE |
| | EESTWMSDDDIRLGWAVIGSRSGSTPLFFSRPEGGGNGVRFPGKSQIGDRGSALFKDQAITAV |
| | NQFHNVMAGQPEELSNPNGNNQIFMNQRGSKGVVLANAGSSSVTVNTSTKLPDGRYDNRAG |
| | AGSFQVANGKLTGTINARSAAVLYSDDIGNAPHVFLENYQTGAVHSFNDQLTVTLRANAKTAK |
| | AVYQINNGQQTAFKDGDRLTIGKGDPIGTTYNIRLTGTNGEGAERTQEYTFVKKDQAQTNIIGY |
| | QNPDHWGQVNAYIYKHDGGRAIELTGSWPGKAMTKNANGIYTLTLPANADTANAKVIFNNGSA |
| | QVPGQNQPGFDYVQNGLYNNSGLNGYLPH |
| 2 | MFQKRFKTSLLPLFAGFLLLFHMVMAGPAAANAETQNTSNELTAPSIKSGTILHAWNWSFNTLK |
| | HNMKDIHDAGYTAIQTSPINQVKEGNQGNKSMSNWYWLYQPTSYQIGNRYLGTEQEFKEMCA |
| | AAEEYGVKVIVDAVINHTTSDYAAISNEIKSIPNWTHGNTQIKNWSDRWDVTQNSLLGLYDWNT |
| | QNTQVQSYLKRFLETALNDGADGFRFDAAKHIELPDDGNYGSQFWPNITNTSAEFQYGEILQD |
| | SASRDAAYANYMNVTASNYGHSIRSALKNRNLSVSNISHYASDVSADKLVTWVESHDTYANDD |
| | EESTWMSDDDIRLGWAVIASRSGSTPLFFSRPEGGGNGVRFPGKTQIGDRGSALFEDQAIAAV |
| | NTFHNVMAGQPEELSNPNGNNQIFMNQRGSQGIVLANAGSSSVSINVSTKLPDGSYDNKAGA |
| | GSFQVRDGKLTGTLNARSVAVLYPDDIANAPQVSLENYKTGVTHFFNDQLTITLRANANTTKAV |
| | YQINNGQETAFKDGDQLTIGKGDPFGKTYNITLTGTNSDGVTRTQEYSFVKRDPSSAKTIGYQN |
| | PNHWGQVNAYIYKHDGGRAIELTGSWPGKAMTKNADGIYTLTLPADTDTTNAKVIFNNGSAQV |
| | PGQNQPGFDYVQNGLYNDSGLSGSLPQ |
| 3 | MEVNVKGNYVQVYVMLPLDAVSVNNRFEKGDELRAQLRKLVEAGVDGVMVDVWWGLVEGK |
| | GPKAYDWSAYKQLFELVQKAGLKLQAIMSFHQCGGNVGDAVNIPIPQWVRDVGTRDPDIFYTD |
| | GHGTRNIEYLTLGVDNQPLFHGRSAVQMYADYMTSFRENMKEFLDAGVIVDIEVGLGPAGEM |
| | RYPSYPQSHGWSFPGIGEFICYDKYLQADFKAAAAAVGHPEWEFPNDAGQYNDTPERTQFFR |
| | DNGTYLTEKGRFFLAWYSNNLIKHGDRILDEANKVFLGYKVQLAIKISGIHWWYKVPSHAAELT |
| | AGYYNLHDRDGYRTIARMLKRHRASINFTCAEMRDSEQSSQAMSAPEELVQQVLSAGWREGL |
| | NVACENALPRYDPTAYNTILRNARPHGINQSGPPEHKLFGFTYLRLSNQLVEGQNYVNFKTFV |
| | DRMHANLPRDPYVDPMAPLPRSGPEISIEMILQAAKPKLQPFPFQEHTDLPVGPTGGMGGQAE |
| | GPTCGMGGQVKGPTGGMGGQAEDPTSGMGGELPATM |
| 4 | MAPIPGVMPIGNYVSLYVMLPLGVVNADNVFPDKEKVEDELKQVKAGGCDGVMVDVWWGIIE |
| | AKGPKQYDWSAYRELFQLVKKCGLKIQAIMSFHQCGGNVGDAVFIPIPQWILQIGDKNPDIFYT |
| | NRAGNRNQEYLSLGVDNQRLFQGRTALEMYRDFMESFRDNMADFLKAGDIVDIEVGCGAAGE |
| | LRYPSYPETQGWVFPGIGEFQCYDKYMVADWKEAVKQAGNADWEMPGKGAGTYNDTPDKT |
| | EFFRPNGTYKTDMGKFFLTWYSNKLIIHGDQVLEEANKVFVGLRVNIAAKVSGIHWWYNHVSH |
| | AAELTAGFYNVAGRDGYRPIARMLARHHATLNFTCLEMRDSEQPAEAKSAPQELVQQVLSSG |
| | WKEYIDVAGENALPRYDATAYNQMLLNVRPNGVNLNGPPKLKMSGLTYLRLSDDLLQTDNFEL |
| | FKKFVKKMHADLDPSPNAISPAVLERSNSAITIDELMEATKGSRPFPWYDVTDMPVDGSNPFD |
| 5 | MAPSVTLPLTTAILAIARLVAAQQPGTSTPEVHPKLTTYKCTKSGGCVAQDTSVVLDWNYRWM |
| | HDANYNSCTVNGGVNTTLCPDEATCGKNCFIEGVDYAASGVTTSGSSLTMNQYMPSSSGGYS |
| | SVSPRLYLLDSDGEYVMLKLNGQELSFDVDLSALPCGENGSLYLSQMDENGGANQYNTAGAN |
| | YGSGYCDAQCPVQTWRNGTLNTSHQGFCCNEMDILEGNSRANALTPHSCTATACDSAGCGF |
| | NPYGSGYKSYYGPGDTVDTSKTFTIITQFNTDNGSPSGNLVSITRKYQQNGVDIPSAQPGGDTI |
| | SSCPSASAYGGLATMGKALSSGMVLVFSIWNDNSQYMNWLDSGNAGPCSSTEGNPSNILAN |
| | NPNTHVVFSNIRWGDIGSTTNSTAPPPPPASSTTFSTTRRSSTTSSSPSCTQTHWGQCGGIGY |
| | SGCKTCTSGTTCQYSNDYYSQCL |
| 6 | MKSRYKRLTSLALSLSMALGISLPAWASPDTSVDNKVNFSTDVIYQIVTDRFADGDRTNNPAGD |
| | AFSGDRSNLKLYFGGDWQGIIDKINDGYLTGMGVTALWISQPVENITSVIKYSGVNNTSYHGYW |
| | ARDFKQTNDAFGDFADFQNLIDTLTLITSRSDRLRPQPHVSGRAGTNPGFAENGALYDNGSLL |
| | GAYSNDTAGLFHHNGGTDFSTIEDGIYKNLYDLADINHNNNAMDAYFKSAIDLWLGMGVDGIRF |
| | DAVKQYPFGWQKSFVSSIYGGDHPVFTFGEWYLGADQTDGDNIKFANESGMNLLDFEYAQEV |
| | REVFRDKTETMKDLYEVLASTESQYDYINNMVTFIDNHDMDRFQVAGSGTRATEQALALTLTS |
| | RGVPAIYYGTEQYMTGDGDPNNRAMMTSFNTGTTAYKVIQALAPLRKSNPAIAYGTTTERWVN |
| | NDVLIIERKFGSSAALVAINRNSSAAYPISGLLSSLPAGTYSDVLNGLLNGNSITVGSGGAVTNFT |
| | LAAGGTAVWQYTAPETSPAIGNVGPTMGQPGNIVTIDGRGFGGTAGTVYFGTTAVTGSGIVSW |
| | EDTQIKAVIPKVAAGKTGVSVKTSSGTASNTFKSFNVLTGDQVTVRFLVNQANTNYGTNVYLVG |
| | NAAELGTWDPNKAIGPMYNQVIAKYPSWYYDVSVPAGTKLDFKFIKKGGGTVTWEGGGNHTY |
| | TTPASGVGTVTVDWQN |
| 7 | MKRWLSVVLSMSLVFSAFFLVSDTQKVTVEAAGNLNKVNFTSDIVYQIVVDRFVDGNTSNNPS |
| | GSLFSSGCTNLRKYCGGDWQGIINKINDGYLTEMGVTAIWISQPVENVFAVMNDADGSTSYHG |
| | YWARDFKKTNPFFGTLSDFQRLVDAAHAKGIKVIIDFAPNHTSPASETNPSYMENGRLYDNGTL |
| | IGGYTNDTNSYFHHNGGTTFSNLEDGIYRNLFDLADFNHQNQFIDKYLKDAIKLWLDMGIDGIR |
| | MDAVKHMPFGWQKSFMDEVYDYRPVFTFGEWFLSENEVDSNNHFFANESGMSLLDFRFGQK |
| | LRQVLRNNSDDWYGFNQMIQDTASAYDEVIDQVTFIDNHDMDRFMADEGDPRKVDIALAVLLT |
| | SRGVPNIYYGTEQYMTGNGDPNNRKMMTSFNKNTRAYQVIQKLSSLRRSNPALSYGDTEQR |
| | WINSDVYIYERQFGKDVVLVAVNRSLSKSYSITGLFTALPSGTYTDQLGALLDGNTIQVGSNGA |
| | VNAFNLGPGEVGVWTYSAAESVPIIGHIGPMMGQVGHKLTIDGEGFGTNVGTVKFGNTVASVV |
| | SWSNNQITVTVPNIPAGKYNITVQTSGGQVSAAYDNFEVLTNDQVSVRFVVNNANTNWGENIY |
| | LVGNVHELGNWNTSKAIGPLFNQVIYSYPTWYVDVSVPEGKTIEFKFIKKDGSGNVIWESGSNH |
| | VYTTPTSTTGTVNVNWQY |
| 8 | MKDMVRERLEKEELASLLQDWVEHYRPFSVNGKSANYIPALGEINDSQLGICILEPDGSMIHAG |
| | EWNVPFTLQSISKVISFIAACMSRGIPYVLDRVDVEPTGDAFNSIIRLEIHKPGKPFNPMINAGAIT |
| | VASLLPGETAHNKLEYLFSVFEKLIGKRPHIHEEVFRSEWQTAHRNRALAYYLKETNFLEADVE |
| | ETLEVYLKQCAMESTTEDIALIGLILAHDGYHPIRREQVIPKDVAKLAKALMLTCGMYNASGKYA |
| | AFVGVPAKSGVSGGIMALVPPSARREQPFHNGCGIGIYGPAIDEYGNSLTGGMLLKHIAQEWE |
| | LSIF |
| 9 | MTAVNQTISKVVNGKRVITKKPELLAPAGNLEKLKIAVHYGADAVFIGGQEYGLRSNADNFSIEE |
| | IAEGVEFAKKYGAKIYVTTNIFAHNENMDGLEEYLKALGDAKVAGIIVADPLIIETCRRVAPDVEIH |
| | LSTQQSLSNWKAVQFWKEEGLDRVVLARETSGLEIKEMKEKVDIEIETFIHGAMCIAYSGRCVL |
| | SNHMTARDSNRGGCCQSCRWDYDLYQTDGANAVALYDEEDAPFAMSPKDLKLIESIPQMIEM |
| | GIDSLKIEGRMKSIHYVATVVSVYRKVIDAYCADPENFVIQKEWLDELDKCANRDTAPAFFEGTP |
| | GYEEQMFGEHGKKTTFDFAGLVLAYDEETQMVTLQQRNFFKQGDEVEFFGPEIDNFTFTIGTI |
| | WDEDGNELDAARHPLQIVRFKVDKKIYPSNMMRKGK |
| 10 | MVSIRRSFEAYVDDMNIITVLIPAEQKEIMTPPFRLETEITDFPLAVREEYSLEAKYKYVCVSDHP |
| | VTFGKIHCVRASSGHKTDLQIGAVIRTAAFDDEFYYDGELGAVYTADHTVFKVWAPAATSAAVK |
| | LSHPNKSGRTFQMTRLEKGVYAVTVTGDLHGYEYLFCICNNSEWMETVDQYAKAVTVNGEKG |
| | VVLRPDQMKWTAPLKPFSHPVDAVIYETHLRDFSIHENSGMINKGKYLALTETDTQTANGSSS |
| | GLAYVKELGVTHVELLPVNDFAGVDEEKPLDAYNWGYNPLHFFAPEGSYASNPHDPQTRKTE |
| | LKQMINTLHQHGLRVILDVVFNHVYKRENSPFEKTVPGYFFRHDECGMPSNGTGVGNDIASER |
| | RMARKFIADCVVYWLEEYNVDGFRFDLLGILDIDTVLYMKEKATKAKPGILLFGEGWDLATPLP |
| | HEQKAALANAPRMPGIGFFNDMFRDAVKGNTFHLKATGFALGNGESAQAVMHGIAGSSGWK |
| | ALAPIVPEPSQSINYVESHDNHTFWDKMSFALPQENDSRKRSRQRLAAAIILLAQGVPFIHSGQ |
| | EFFRTKQGVENSYQSSDSINQLDWDRRETFKEDVHYIRRLISLRKAHPAFRLRSAADIQRHLEC |
| | LTLKEHLIAYRLYDLDEVDEWKDIIVIHHASPDSVEWRLPNDIPYRLLCDPSGFQEDPTEIKKTVA |
| | VNGIGTVILYLASDLKSFA |
| 11 | MLRYTCHALFLGSLVLLSGCDNSSSSSTSGSPGSPGNPGNPGTPGTPDPQDVVVRLPDVAVP |
| | GEAVQASARQAVIHLVDIAGITSSTPADYATKNLYLWNNETCDALSAPVADWNDVSTTPTGSD |
| | KYGPYWVIPLTKESGCINVIVRDGTNKLIDSDLRVSFSDFTDRTVSVIAGNSAVYDSRADAFRAA |
| | FGVALADAHWVDKTTLLWPGGENKPIVRLYYSHSSKVAADSNGEFTDKYVKLTPTTVSQQVS |
| | MRFPHLASYPAFKLPDDVNVDELLQGETVAISAESDGILSSATQVQTAGVLDDTYAAAAEALSY |
| | GAQLTDSGVTFRVWAPTAQQVELVVYSADKKVVASHPMTRDSASGAWSWQGGSDLKGAFY |
| | RYAMTVYHPQSRKVEQYEVTDPYAHSLSTNSEYSQVVDLNDSALKPEGWDGLTMPHAQKTK |
| | ADLAKMTIHESHIRDLSAWDQTVPAELRGKYLALTAQESNMVQHLKQLSASGVTHIELLPVFDL |
| | ATVNEFSDKVADIQQPFSRLCEINSAVKSSEFAGYCDSGSTVEEVLTQLKQNDSKDNPQVQAL |
| | NTLVAQTDSYNWGYDPFHYTVPEGSYATDPEGTARIKEFRTMIQAIKQDLGMNVIMDVVYNHT |
| | NAAGPTDRTSVLDKIVPWYYQRLNETTGSVESATCCSDSAPEHRMFAKLIADSLAVWTTDYKI |
| | DGFRFDLMGYHPKAQILSAWERIKALNPDIYFFGEGWDSNQSDRFEIASQINLKGTGIGTFSDR |
| | LRDAVRGGGPFDSGDALRQNQGVGSGAGVLPNELTTLSDDQARHLADLTRLGMAGNLADFV |
| | LIDKDGAVKKGSEIDYNGAPGGYAADPTEVVNYVSKHDNQTLWDMISYKAAQEADLDTRVRM |
| | QAVSLATVMLGQGIAFDQQGSELLRSKSFTRDSYDSGDWFNRVDYSLQDNNYNVGMPRSSD |
| | DGSNYDIIARVKDAVATPGETELKQMTAFYQELTALRKSSPLFTLGDGATVMKRVDFRNTGTD |
| | QQTGLLVMTIDDGMQAGASLDSRVDGIVVAINAAPESRTLQDFAGTSLQLSAIQQAAGDRSLAS |
| | GVQVAADGSVTLPAWSVAVLELPQGESQGAGLPVSSK |
| 12 | MSQRGRTLVFAALGAVMCTTALMPSAGAATGSGSGSGTGEEKRSYAETHRLTADDVDDINAL |
| | NESAPAASSAGPSFRAPDSDERVTPPAEPLDRMPDPYRPSYGRAETIVNNYIRKVVQQVYSHR |
| | DGRKQQMTEEQREWLSYGCVGVTWVNSGQYPTNRLAFAFFDEDKYKNELKNGRPRSGETR |
| | AEFEGRVAKDSFDEAKGFQRARDVASVMNKALENAHDEGAYLDNLKKELANGNDALRNEDAR |
| | SPFYSALRNTPSFKDRNGGNHDPSKMKAVIYSKHFWSGQDRSGSSDKRKYGDPEAFRPDRG |
| | TGLVDMSRDRNIPRSPTSPGESFVNFDYGWFGAQTEADADKTVWTHGNHYHAPNGSLGAMH |
| | VYESKFRNWSDGYSDFDRGAYVVTFVPKSWNTAPDKVTQGWP |
| 13 | MNKLAVLYAEHIATLQKRTREIIERENLDGVVFHSGQAKRQFLDDMYYPFKVNPQFKAWLPVID |
| | NPHCWIVANGTDKPKLIFYRPVDFWHKVPDEPNEYWADYFDIELLVKPDQVEKLLPYDKARFA |
| | YIGEYLEVAQALGFELMNPEPVMNFYHYHRAYKTQYELACMREANKIAVQGHKAARDAFFQG |
| | KSEFEIQQAYLLATQHSENDNAYGNIVALNENCAILHYTHFDRVAPATHRSFLIDAGANFNGYA |
| | ADITRTYDFTGEGEFAELVATMKQHQIALCNQLAPGKLYGELHLDCHQRVAQTLSDFNIVDLSA |
| | DEIVAKGITSTFFPHGLGHHIGLQVHDVGGFMADEQGAHQEPPEGHPFLRCTRKIEANQVFTIE |
| | PGLYFIDSLLGDLAATDNNQHINWDKVAELKPFGGIRIEDNIIVHEDSLENMTRELRARLTTHSLR |
| | GLSAPQFSINDPAVMSEYSYPSEPLSYEEEIKKSTFIVHVRTRRILVRRRTLSPILIAVTPMPAITA |
| | GLM |
| 14 | MVQIKVAALAMLFASQVLSEPIEPRQASVSIDTKFKAHGKKYLGNIGDQYTLTKNSKTPAIIKADF |
| | GALTPENSMKWDATEPSRGQFSFSGSDYLVNFAQSNNKLIRGHTLVWHSQLPSWVQSITDKN |
| | TLIEVMKNHITTVMQHYKGKIYAWDVVNEIFNEDGSLRDSVFYKVIGEDYVRIAFETARAADPNA |
| | KLYINDYNLDSASYPKLTGMVSHVKKWIAAGIPIDGIGSQTHLSAGGGAGISGALNALAGAGTKE |
| | IAVTELDIAGASSTDYVEVVEACLNQPKCIGITVWGVADPDSWRSSSTPLLFDSNYNPKPAYTAI |
| | ANAL |
| 15 | MFKFKKKFLVGLTAAFMSISMFSATASAAGTDYWQNWTDGGGTVNAVNGSGGNYSVNWSNT |
| | GNFVVGKGWTTGSPFRTINYNAGVWAPNGNGYLTLYGWTRAPLIEYYVVDSWGTYRPTGTYK |
| | GTVKSDGGTYDIYTTTRYNAPSIDGDNTTFTQYWSVRQSKRPTGSNAAITFSNHVNAWKSHG |
| | MNLGSNWAYQVLATEGYKSSGSSNVTVW |
| 16 | MIPNITQLKTAALVMLFAGQALSGPVESRQASESIDAKFKAHGKKYLGNIADQGTLNGNPKTPAI |
| | IKANFGQLSPENSMKWDATEPSQGQFSFAGSDYFVEFAETNGKLIRGHTLVWHSQLPSWVSSI |
| | TDKTTLTDVMKNHITTVMKQYKGKLYAWDVVNEIFEEDGTLRDSVFSRVLGEDFVRIAFETARE |
| | ADPEAKLYINDYNLDSATSAKLQGMVSHVKKWIAAGVPIDGIGSQTHLGAGAGAAASGALNALA |
| | SAGTEEVAVTELDIAGATSTDYVDVVNACLDQPKCVGITVWGVADPDSWRADESPLLFDASYN |
| | PKEAYNVSQLLSRQHAFDLYLKLGNLLLSRLHSD |
| 17 | MLLEAIFHEAKGSYAYPISETQLRVRLRAKKGDVVRCEVLYADRYASPEEELAHALAGKAGSDE |
| | RFDYFEALLECSTKRVKYVFLLTGPQGEAVYFGETGFSAERSKAGVFQYAYIHRSEVFTTPEW |
| | AKEAVIYQIFPERFANGDPSNDPPGTEQWAKDARPRHDSFYGGDLKGVIDRLPYLEELGVTAL |
| | YFTPIFASPSHHKYDTADYLAIDPQFGDLPTFRRLVDEAHRRGIKIILDAVFNHAGDQFFAFRDV |
| | LQKGEQSRYKDWFFIEDFPVSKTSRTNYETFAVQVPAMPKLRTENPEVKEYLFDVARFWMEQ |
| | GIDGWRLDVANEVDHAFWREFRRLVKSLNPDALIVGEIWHDASGWLMGDQFDSVMNYLFRES |
| | VIRFFATGEIHAERFDAELTRARMLYPEQAAQGLWNLLDSHDTERFLTSCGGNEAKFRLAVLF |
| | QMTYLGTPLIYYGDEIGMAGATDPDCRRPMIWEEKEQNRGLFEFYKELIRLRHRLASLTRGNV |
| | RSWHADKQANLYAFVRTVQDQHVGVVLNNRGEKQTVLLQVPESGGKTWLDCLTGEEVHGKQ |
| | GQLKLTLRPYQGMILWNGR |
| 18 | MAPSATLPLTTAILAIGRLVAAQQPGTSTPEVHPKLTTYKCTTSGGCVAQDTSVVLDWNYRWM |
| | HDANYNSCTVNGGVNTTLCPDEATCGKNCYIEGVDYAASGVTASGSTLTLNQYMPSSSGGYS |
| | SVSPRLYLLGPDGEYVMLKLNGQELSFDVDLSALPCGENGSLYLSQMDENGGANQYNTAGAN |
| | YGSGYCDAQCPVQTWRNGTLNTSGQGFCCNEMDILEGNSRANALTPHSCTATACDSAGCGF |
| | NPYGSGYPNYFGPGDTVDTSKTFTIITQFNTDNGSPSGNLVSITRKYRQNGVDIPSAKPGGDTI |
| | SSCPSASAYGGLATMGKALSSGMVLVFSIWNDNSQYMNWLDSGRAGPCSSTEGNPSNILAN |
| | NPGTHVVYSNIRWGDIGSTTNSTGGNPPPPPPPASSTTFSTTRRSSTTSSSPSCTQTHWGQC |
| | GGIGYTGCKTCTSGTTCQYGNDYYSQCL |
| 19 | MKRSISIFITCLLMTVLTISGFAVPEASAAGAKTPVALNGQLSIKGTQLVNQNGKPVQLKGISSHG |
| | LQWFGDYVNKDTLKWLRDDWGITVFRAAMYTADGGYIENPSVKNKVKEAVEAAKELGIYVIID |
| | WHILNDGNPNQNKEKAKEFFKEMSSLYGSSPNVIYEIANEPNGDVNWKRDIKPYAEEVISVIRK |
| | NDPDNIIITGTGTWSQDVNDAADDQLKDANVMYALHFYAGTHGQFLRDKADYALSKGAPIFVT |
| | EWGTSDASGNGGVYLDQSREWLNYLDSKKISWVNWNLSDKQESSSALKPGASKTGGWPLS |
| | DLSASGTFVRENIRGSQNSREDRSETPKQEKPAQENSISVQYRTGDGSVNSNQIRPQINVKNN |
| | SKTTVNLKNVTVRYWYNTKNKGQNFDCDYAKIGCSNVTHKFVTLHKPVKGADAYLELGFRNG |
| | TLSPGASTGEIQIRLHNEDWSNYSQAGDYSFFQSNTFKDTKKITLYHQGKLI |
| 20 | MSFQPTPEDRFTFGLWTVGWQGRDPFGDATRPALDPVETVQRLAELGAYGVTFHDDDLIPFG |
| | SSDTERESHIKRFRQALDATGMTVPMATTNLFTHPVFKDGGFTANDRDVRRYALRKTIGNIDLA |
| | AELGAKTYVAWGGREGAESGGAKDVRDALDRMKEAFDLLGEYVTAQGYDLRFAIEPKPNEPR |
| | GDILLPTVGHALAFIERLERPELYGVNPEVGHEQMAGLNFPHGIAQALWAGKLFHIDLNGQSGI |
| | KYDQDLRFGAGDLRAAFWLVDLLETAGYEGPRHFDFKPPRTEDFDGVWASAAGCMRNYLILK |
| | DRAAAFRADPEVQEALRAARLDQLAQPTAADGLDALLADRAAFEDFDVDAAAARGMAFEHLD |
| | QLAMDHLLGARG |
| 21 | MLNPKVAYMVWMTCLGLTLPSQAQSNDYRPSYHFTPDQYWMNEPNGLIKIGSTWHLFFQHN |
| | PTANVWGNICWGHATSTDLMHWAHKPTAIADENGVEAFTGTAYYDPNNTSGLGDSANPPYLA |
| | WFTGYTTSSQTQDQRLAFSVDNGATWTKFQGNPIISTSQEAPHDITGGLESRDPKVFFHRQSG |
| | NWIMVLAHGGQDKLSFWTSADTINWTWQSDLKSTSINGLSSDITGWEVPDMFELPVEGTEETT |
| | WVVMMTPAEGSPAGGNGVLAITGSFDGKSFTADPVDASTMWLDNGRDFDGALSWVNVPASD |
| | GRRIIAAVMNSYGSNPPTTTWKGMLSFPRTLSLKKVGTQQHFVQQPITELDTISTSLQILANQTT |
| | TPGQTLLSSIRGTALDVRVAFYPDAGSVLSLAVRKGASEQTVIKYTQSDATLSVDRTESGDISY |
| | DPAAGGVHTAKLEEDGTGLVSIRVLVDTCSVEVFGGQGEAVISDLIFPSDSSDGLALEVTGGNA |
| | VLQSVDVRSVSLE |
| 22 | MSCLIPENLRNPKKVHENRLPTRAYYYDQDIFESLNGPWAFALFDAPLDAPDAKNLDWETAKK |
| | WSTISVPSHWELQEDWKYGKPIYTNVQYPIPIDIPNPPTVNPTGVYARTFELDSKSIESFEHRLR |
| | FEGVDNCYELYVNGQYVGFNKGSRNGAEFDIQKYVSEGENLVVVKVFKWSDSTYIEDQDQW |
| | WLSGIYRDVSLLKLPKKAHIEDVRVTTTFVDSQYQDAELSVKVDVQGSSYDHINFTLYEPEDGS |
| | KVYDASSLLNEENGNTTFSTKEFISFSTKKNEETAFKINVKAPEHWTAENPTLYKYQLDLIGSDG |
| | SVIQSIKHHVGFRQVELKDGNITVNGKDILFRGVNRHDHHPRFGRAVPLDFVVRDLILMKKFNIN |
| | AVRNSHYPNHPKVYDLFDKLGFWVIDEADLETHGVQEPFNRHTNLEAEYPDTKNKLYDVNAHY |
| | LSDNPEYEVAYLDRASQLVLRDVNHPSIIIWSLGNEACYGRNHKAMYKLIKQLDPTRLVHYEGD |
| | LNALSADIFSFMYPTFEIMERWRKNHTDENGKFEKPLILCEYGHAMGNGPGSLKEYQELFYKE |
| | KFYQGGFIWEWANHGIEFEDVSTADGKLHKAYAYGGDFKEEVHDGVFIMDGLCNSEHNPTPG |
| | LVEYKKVIEPVHIKIAHGSVTITNKHDFITTDHLLFIDKDTGKTIDVPSLKPEESVTIPSDTTYVVAV |
| | LKDDAGVLKAGHEIAWGQAELPLKVPDFVTETAEKAAKINDGKRYVSVESSGLHFILDKLLGKIE |
| | SLKVKGKEISSKFEGSSITFWRPPTNNDEPRDFKNWKKYNIDLMKQNIHGVSVEKGSNGSLAV |
| | VTVNSRISPVVFYYGFETVQKYTIFANKINLNTSMKLTGEYQPPDFPRVGYEFWLGDSYESFEW |
| | LGRGPGESYPDKKESQRFGLYDSKDVEEFVYDYPQENGNHTDTHFLNIKFEGAGKLSIFQKEK |
| | PFNFKISDEYGVDEAAHACDVKRYGRHYLRLDHAIHGVGSEACGPAVLDQYRLKAQDFNFEFD |
| | LAFE |
| 23 | MRSLLILVLCFLPLAALGKVFGRCELAAAMKRHGLDNYRGYSLGNWVCAAKFESNFNTQATNR |
| | NTDGSTDYGILQINSRWWCNDGRTPGSRNLCNIPCSALLSSDITASVNCAKKIVSDGNGMNAW |
| | VAWRNRCKGTDVQAWIRGCRL |
| 24 | MHYKLLFAAAAASLASAVSAAGVVGAAEGFAHGVTGGGSASPVYPTTTDELVSYLGDNEPRVII |
| | LDRTFDFTGTEGTETTTGCAPWGTASQCQVAINLHSWCDNYQASAPKVSVTYDKAGILPITVN |
| | SNKSIVGQGTKGVIKGKGLRVVSGAKNVIIQNIAVTDINPKYVWGGDAITVDDSDLVWIDHVTTA |
| | RIGRQHIVLGTSADNRVTISYSLIDGRSDYSATCNGQHYWGVYLDGSNDMVTLKGNYFYNLSG |
| | RMPKVQGNTLLHAVNNLFHNFDGHAFEIGTGGYVLAEGNVFQDVNTVVETPISGQLFSSPDAN |
| | INQQCASVFGRSCQLNAFGNSGSMSGSDTSIISKFAGKTIATAHPPGNIAQWTMKNAGQGK |
| 25 | MKAILIPFLSLLIPLTPQSAFAQSEPELKLESVVIVSRHGVRAPTKATQLMQDVTPDAWPTWPVK |
| | LGWLTPRGGELIAYLGHYQRQRLVADGLLAKKGCPQSGQVAIIADVDERTRKTGEAFAAGLAP |
| | DCAITVHTQADTSSPDPLFNPLKTGVCQLDNANVTDAILSRAGGSIADFTGHRQTAFRELERVL |
| | NFPQSNLCLKREKQDESCSLTQALPSELKVSADNVSLTGAVSLASMLTEIFLLQQAQGMPEPG |
| | WGRITDSHQWNTLLSLHNAQFYLLQRTPEVARSRATPLLDLIMAALTPHPPQKQAYGVTLPTS |
| | VLFIAGHDTNLANLGGALELNWTLPGQPDNTPPGGELVFERWRRLSDNSQWIQVSLVFQTLQ |
| | QMRDKTPLSLNTPPGEVKLTLAGCEERNAQGMCSLAGFTQIVNEARIPACSL |
| 26 | MTISAFERQKLTLRNGLLIAIGTACSLGSTVYASETPASGYQTEKVVILSRHGVRAPTKMTQTMR |
| | DVTPNTWPEWPVKLGYITPRGEQLIRVMGGFYREKFQQQGILSQENCPAPNSVYVwADVDQR |
| | TLKTGEAFLAGLAPQCGLTIHHQQNLEKADPLFHPVKAGICSMEKNQIQQAVEKEAQNPIDNLN |
| | QHYLPSLALMNTTLNFANSAWCQKHSVDKSCDLGQSMPSKLSIQENGNKITLDGAIGLSSTLAE |
| | IFLLEYAQGMPKAAWGNIHSEQEWAALLKLHNTQFDLMARTPYIARHNGTPLLQAISNALDPNA |
| | TASKLPDISPDNKILFIAGHDTNIANISGMLNISWTLPGQPDNTPPGGALVFERLADKSGKQYVS |
| | VSMVYQTLEQLHAQTPLSLKEPAGSVQLKIPGCSDQTAEGYCPLPTFTRMVSQSVEPGCQLQ |
| 27 | MHSYQLLGLAAVGSLVSAAPAPSRVSEFAKKASTCTFTSASEASESISSCSDVVLSGIEVPAGE |
| | TLDLSDAADGSTITFEGTTSFGYKEWKGPLIRFGGKDLTVTMADGAVIDGDGSRWWDSKGTN |
| | GGKTKPKFMYIHDVEDSTFKGINVKNTPVQAISVQATNVHLNDFTIDNSDGDDNGGHNTDGFDI |
| | SESTGVYISGATVKNQDDCIAINSGESISFTGGTCSGGHGLSIGSVGGRDDNTVKNVTISDSTV |
| | SDSANGVRIKTIYKETGDVSEITYSNIQLSGITKYGIVIEQDYENGSPTGTPSTGIPITDVTVDGIT |
| | GTLEDDATQVYILCGDGSCSDWTWSGVDLSGGKASDDCENVPSRFLHHCCSPPSRDSKFDL |
| | TFLVHNGEYPSAVILENKIVSKHWSDEERSVTATDNEENTVEASHYTDEGYTNVTLTEARSFKA |
| | QDVSRARQEILRFETRRLTHDNDMLIAMLQSE |
| 28 | MKQIVKRSHAIRIVAALGIIGLWMFFSSNELSIATPGLIKAKSGIDEVQGAAAEKNDARLKEIEKQT |
| | IMPLMGDDKVKKEVGRASWKYFHTLLARFPDEPTPEEREKLHTFIGLYAELYPCGECSYHFVKL |
| | IEKYPVQTSSRTAAAMWGCHIHNKVNEYLKKDIYDCATILEDYDCGCSDSDGKRVSLEKEAKQ |
| | HG |
| 29 | MVAFSSLFTAFTAIAGSLAAPADLVKNDNVPDLVSRGPSNFVIDSTNNTSLARRSAINYDQDYTT |
| | GGTVNYYSSSTGFEVTWNTQDDFVVGVGWNPGSTTPINFGGQFSVSSGTGLLSVYGWSTSP |
| | LVEYYIIEDYANPPSFGTQKGSLTSDGGTYIIWENTRYNEPSIQGTSTFNQYISVRQSPRTEGTV |
| | TVQNHFNAWKNLGMNLGTLNFQVIAAEGWGGSGSASYAVNNN |
| 30 | MVGFTPVALAALAATGALAFPAGNATELEKRQTTPNSEGWHDGYYYSWWSDGGAQATYTNL |
| | EGGTYEISWGDGGNLVGGKGWNPGLNARAIHFEGVYQPNGNSYLAVYGWTRNPLVEYYIVE |
| | NFGTYDPSSGATDLGTVECDGSIYRLGKTTRVNAPSIDGTQTFDQYWSVRQDKRTSGTVQTG |
| | CHFDAWARAGLNVNGDHYYQIVATEGYFSSGYARITVADVG |
| 31 | MKKLICVLLAYVTMLSLMLTGPAASEVSAASDATVRLSAEKQVIRGFGGMNHPAWIGDLTAAQ |
| | RETAFGNGQNQLGFSILRIHVDENRNNWYREVETAKSAIKHGAIVFASPWNPPSDMVETFNRN |
| | GDTSAKRLRYDKYAAYAKHLNDFVTFMKNNGVNLYAISVQNEPDYAHDWTWWTPQEILRFMK |
| | ENAGSINARVIAPESFQYLKNISDPILNDPKALANMDILGAHLYGTQLNNFAYPLFKQKGAGKDL |
| | WMTEVYYPNSDNNSADRWPEALDVSHHIHNSMVEGDFQAYVWWYIRRSYGPMKEDGTISKR |
| | GYNMAHFSKFVRPGYVRVDATKSPASNVYVSAYKGDNKVVIVAINKNNSGVNQNFVLQNGSV |
| | SQVSRWITSSSSNLQPGTNLNVTDNHFWAHLPAQSVTTFVANLR |
| 32 | MIQAPARPKSRRRVRARLLASGVSAVAMVAATTVLPGTAHAAITTNQTGWHNGYFYSFWTDS |
| | QGTVSMELGSGGNYSTSWYNTGNFVAGKGWNPGGRRTVNYSASYSPSGNSYLTLYGWTRN |
| | PLVEYYIVDSWGSWRPTGTYKGTITSDGGTYDIYQTMRYNAPSIEGIRTFPQYWSVRQSKRTS |
| | GTITSGNHFDAWARYGMNLGSHDYMIMATEGYQSSGYSNVTVW |
| 33 | MVQIKAAALAVLFASNVLANPIEPRQASVSIDAKFKAHGKKYLGTIGDQYTLNKNAKTPAIIKADF |
| | GQLTPENSMKWDATEPNRGQFSFSGSDYLVNFAQSNGKLIRGHTLVWHSQLPSWVQSIYDK |
| | GTLIQVMQNHIATVMQRYKGKVYAWDVVNEIFNEDGSLRQSHFYNVIGEDYVRIAFETARAVD |
| | PNAKLYINDYNLDSASYPKLTGLVNHVKKWVAAGVPIDGIGSQTHLSAGAGAAVSGALNALAG |
| | AGTKEVAITELDIAGASSTDYVNVVKACLNQPKCVGITVWGVADPDSWRSSSSPLLFDSNYNP |
| | KAAYTAIANAL |
| 34 | MVAFSSLICALTSIASTLAMPTGLEPESSVNVTERGMYDFVLGAHNDHRRRASINYDQNYQTG |
| | GQVSYSPSNTGFSVNWNTQDDFVVGVGWTTGSSAPINFGGSFSVNSGTGLLSVYGWSTNPL |
| | VEYYIMEDNHNYPAQGTVKGTVTSDGATYTIWENTRVNEPSIQGTATFNQYISVRNSPRTSGT |
| | VTVQNHFNAWASLGLHLGQMNYQVVAVEGWGGSGSASQSVSN |
| 35 | MRSSLVLFFVSAWTALASPIRREVSQDLFNQFNLFAQYSAAAYCGKNNDAPAGTNITCTGNAC |
| | PEVEKADATFLYSFEDSGVGDVTGFLALDNTNKLIVLSFRGSRSIENWIGNLNFDLKEINDICSG |
| | CRGHDGFTSSWRSVADTLRQKVEDAVREHPDYRVVFTGHSLGGALATVAGADLRGNGYDID |
| | VFSYGAPRVGNRAFAEFLTVQTGGTLYRITHTNDIVPRLPPREFGYSHSSPEYWIKSGTLVPVT |
| | RNDIVKIEGIDATGGNNQPNIPDIPAHLWYFGLIGTCL |
| 36 | MACLFYSSCALALALGQSALAAPNLSPRATVSLGSWLATETAVSLNGILDNIGASGAYAQSASA |
| | GIVIASPSTSNPNYYYTWTRDSALTLKVLIDVFRNGNLSLQTVIEEYVDAQAHLQTVTNPSGDLS |
| | EGAGLGEPKFNVDMSAFTDSWGRPQRDGPALRAIALIDFGNWLIENGYSTYAVTNIWPIVRND |
| | LSYVAEYWSQSGYDLWEEVNSMSFFTVANQHRALVEGSTFAGKVGASCSWCDSQAPQILCY |
| | MQSFWTGSYINANTGGGRSGKDANTVLASIHTFDPEATCDDITFQPCSPRALANHKVYTDSFR |
| | SVYSLNSGIAEGVAVAVGRYPEDTYYNGNPWFLSNLAAAEQLYDAIYQWNKIGSISITSTSLAFF |
| | KDIYSSAAVGTYSSSTSTFTDIIDAVTTYADGYISIVQAHAMTNGSLSEQYDKSSGTELSARDLT |
| | WSYAAVLTANMRRSGIVPPSWGATSANTVPSSCSTGSATGTYSTPTATSWPSSLTSGTGATS |
| | TSTSTSTASVSSANHSTTTTTKATTTSSSCSNPTSVAVTFDEIATTSYGENIYIVGSISQLGDWD |
| | TDSAIQLSASKYTTSNHLWYATISLPAGTSFEYKYIRKESDGSITWESDPNRSYTVPLECAVTTA |
| | TESDTWR |
| 37 | MPKKKRKVGRVCRISSLRYRGPGIATMDKKYSIGLAIGTNSVGWAVITDEYKVPSKKFKVLGNT |
| | DRHSIKKNLIGALLFDSGETAEATRLKRTARRRYTRRKNRICYLQEIFSNEMAKVDDSFFHRLEE |
| | SFLVEEDKKHERHPIFGNIVDEVAYHEKYPTIYHLRKKLVDSTDKADLRLIYLALAHMIKFRGHFLI |
| | EGDLNPDNSDVDKLFIQLVQTYNQLFEENPINASGVDAKAILSARLSKSRRLENLIAQLPGEKKN |
| | GLFGNLIALSLGLTPNFKSNFDLAEDAKLQLSKDTYDDDLDNLLAQIGDQYADLFLAAKNLSDAI |
| | LLSDILRVNTEITKAPLSASMIKRYDEHHQDLTLLKALVRQQLPEKYKEIFFDQSKNGYAGYIDG |
| | GASQEEFYKFIKPILEKMDGTEELLVKLNREDLLRKQRTFDNGSIPHQIHLGELHAILRRQEDFY |
| | PFLKDNREKIEKILTFRIPYYVGPLARGNSRFAWMTRKSEETITPWNFEEVVDKGASAQSFIER |
| | MTNFDKNLPNEKVLPKHSLLYEYFTVYNELTKVKYVTEGMRKPAFLSGEQKKAIVDLLFKTNRK |
| | VTVKQLKEDYFKKIECFDSVEISGVEDRFNASLGTYHDLLKIIKDKDFLDNEENEDILEDIVLTLTL |
| | FEDREMIEERLKTYAHLFDDKVMKQLKRRRYTGWGRLSRKLINGIRDKQSGKTILDFLKSDGFA |
| | NRNFMQLIHDDSLTFKEDIQKAQVSGQGDSLHEHIANLAGSPAIKKGILQTVKVVDELVKVMGR |
| | HKPENIVIEMARENQTTQKGQKNSRERMKRIEEGIKELGSQILKEHPVENTQLQNEKLYLYYLQ |
| | NGRDMYVDQELDINRLSDYDVDAIVPQSFLKDDSIDNKVLTRSDKNRGKSDNVPSEEVVKKMK |
| | NYWRQLLNAKLITQRKFDNLTKAERGGLSELDKAGFIKRQLVETRQITKHVAQILDSRMNTKYD |
| | ENDKLIREVKVITLKSKLVSDFRKDFQFYKVREINNYHHAHDAYLNAVVGTALIKKYPKLESEFV |
| | YGDYKVYDVRKMIAKSEQEIGKATAKYFFYSNIMNFFKTEITLANGEIRKRPLIETNGETGEIVWD |
| | KGRDFATVRKVLSMPQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYKEVKKDLIIKLPKYSLFE |
| | LENGRKRMLASAGELQKGNELALPSKYVNFLYLASHYEKLKGSPEDNEQKQLFVEQHKHYLD |
| | EIIEQISEFSKRVILADANLDKVLSAYNKHRDKPIREQAENIIHLFTLTNLGAPAAFKYFDTTIDRKR |
| | YTSTKEVLDATLIHQSITGLYETRIDLSQLGGDAYPYDVPDYASLGSGSPKKKRKVEDPKKKRK |
| | VDGIGSGSNGSSGSNGPTDAAEEELLSKNYHLENEVARLKKGSGSGGSGSGGSGSGGSGSG |
| | GSGGSGSGGSGSGGSGSGGSGSGEELLSKNYHLENEVARLKKGSGSGGSGSGGSGSGGS |
| | GSGGSGGSGSGGSGSGGSGSGGSGSGEELLSKNYHLENEVARLKKGSGSGGSGSGGSGS |
| | GGSGSGGSGGSGSGGSGSGGSGSGGSGSGEELLSKNYHLENEVARLKK |
| 38 | MARILAFDIGISSIGWAFSENDELKDCGVRIFTKVENPKTGESLALPRRLARSARKRLARRKARL |
| | NHLKHLIANEFKLNYEDYQSFDESLAKAYKGSLISPYELRFRALNELLSKQDFARVILHIAKRRGY |
| | DDIKNSDDKEKGAILKAIKQNEEKLANYQSVGEYLYKEYFQKFKENSKEFTNVRNKKESYERCI |
| | AQSFLKDELKLIFKKQREFGFSFSKKFEEEVLSVAFYKRALKDFSHLVGNCSFFTDEKRAPKNS |
| | PLAFMFVALTRIINLLNNLKNTEGILYTKDDLNALLNEVLKNGTLTYKQTKKLLGLSDDYEFKGEK |
| | GTYFIEFKKYKEFIKALGEHNLSQDDLNEIAKDITLIKDEIKLKKALAKYDLNQNQIDSLSKLEFKD |
| | HLNISFKALKLVTPLMLEGKKYDEACNELNLKVAINEDKKDFLPAFNETYYKDEVTNPVVLRAIK |
| | EYRKVLNALLKKYGKVHKINIELGGGSGGYIARLVLNYTKDYLDFLPLSDDENTKLNDTQKGSK |
| | VHVEAKSGMLTSALRHTWGFSAKDRNNHLHHAIDAVIIAYANNSIVKAFSDFKKEQESNSAELY |
| | AKKISELDYKNKRKFFEPFSGFRQKVLDKIDEIFVSKPERKKPSGALHEETFRKEEEFYQSYGG |
| | KEGVLKALELGKIRKVNGKIVKNGDMFRVDIFKHKKTNKFYAVPIYTMDFALKVLPNKAVARSKK |
| | GEIKDWILMDENYEFCFSLYKDSLILIQTKDMQEPEFVYYNAFTSSTVSLIVSKHDNKFETLSKN |
| | QKILFKNANEKEVIAKSIGIQNLKVFEKYIVSALGEVTKAEFRQREDFKK |

The list of enzyme variants together with the respective mutations introduced to the wild-type amino acid sequence is set forth in Table 3.

TABLE 3
List of enzyme variants and the mutations introduced
to the wild-type sequence of the respective enzyme.

| Variant | Enzyme | |
|---|---|---|
| number | number | Mutations |
| 1 | 1 | K7N, L18M, V24I, T35S, N40S, V42D, A43N, N49D, T51N, T64K, Q65E, K68Q, |
| | | R71A, Q91H, D93N, R97E, L102H, S107D, E120D, A127E, V135I, D147H, A149N, |
| | | R157N, E194Q, T197E, R201G, F202Y, L203F, R205Q, L207V, N208A, P225T, |
| | | S232G, R233N, T241N, T259A, S278E, S288A, S291E, D296S, S298P, D300S, |
| | | T304L, I327L, G335A, S338A, G339S, E349Y, R356M, G359N, R366E, A369D, |
| | | F371W, T377A, Q381K, Q406Y, S409N, T423N, V424L |
| 2 | 1 | A43T, N49D, T51N, Q65E, R71A, Q91H, D93N, D147H, R233N, S291E, D296S, |
| | | I327M, E349Y, R356M, R366E, A369D, T377A, Q406Y |
| 3 | 1 | L18M, V42D, A43T, N49D, T51N, Q65E, R71A, Q91H, D93N, D147H, R157N, |
| | | R233N, S291E, D296S, I327L, E349Y, R356M, G359N, R366E, A369D, T377A, |
| | | Q406Y, S409N, T423D |
| 4 | 1 | L18M, N40S, V42D, A43T, N49D, T51N, T64K, Q65E, R71A, Q91H, D93N, D147H, |
| | | R157N, S232G, R233N, T241N, S288K, S291E, D296S, D300S, I327L, E349Y, |
| | | R356M, G359N, R366E, A369D, T377A, Q381K, Q406Y, S409N, T423D |
| 5 | 1 | L18M, N40S, V42D, A43T, N49D, T51N, T64K, Q65E, R71A, Q91H, D93N, A127E, |
| | | D147H, A149N, R157N, P225T, S232G, R233N, T241N, T259A, S288K, S291E, |
| | | D296S, D300S, I327L, G339S, E349Y, R356M, G359N, R366E, A369D, T377A, |
| | | Q381K, Q406Y, S409N, T423D |
| 6 | 1 | K7N, L18M, V24I, T35S, N40S, V42D, A43N, N49D, T51N, T64K, K68Q, R71A, |
| | | Q91H, D93N, R97E, L102H, S107D, E120D, A127E, V135I, D147H, A149N, R157N, |
| | | E194Q, T197E, R201G, F202Y, R205Q, N208A, P225T, S232G, R233N, T241N, |
| | | T259A, S278E, S288A, S291A, D296S, S298P, D300S, T304L, I327L, G335A, |
| | | S336A, S338A, G339S, E349Y, R356M, G359N, R366E, A369D, F371W, T377A, |
| | | Q381K, Q406Y, S409N, T423N, V424L |
| 7 | 1 | K7N, L18M, V24A, N40S, V42D, A43T, N49D, T51N, T64K, Q65D, K68Q, R71A, |
| | | Q91H, D93N, L102H, A127E, V135I, D147H, A149N, R157N, E194Q, R205Q, |
| | | N208A, P225T, S232G, R233N, T241N, T259A, S291E, D296S, D300S, T304L, |
| | | I327L, G335A, S338A, G339S, E349Y, R356M, G359N, R366E, A369D, T377A, |
| | | Q381K, Q406Y, S409N, T423D |
| 8 | 1 | A43T, N49D, T51N, Q65E, R71A, Q91H, D93N, D147H, R233N, S291E, D296S, |
| | | I327L, E349Y, R356M, R366E, A369D, T377A, Q406Y |
| 9 | 1 | A43T, N49D, T51N, Q65E, R71A, Q91H, D93N, D147H, R233N, S291E, D296S, |
| | | I327L, A369D, T377A, Q406Y |
| 10 | 2 | K48H, S49N, L63A, K64A, H65Q, D72E, K87H, Y100W, S107K, Q109T, R113N, |
| | | Y114Q, T117S, V133L, K134Q, V137A, S197Q, K200I, E204N, T205D, A206C, |
| | | T241N, K281A, S286N, S291G, A294Q, S295N, S340G, V355T, T381H, E392F, |
| | | I402T, I412F, V427T, S428A, S435R, R448N, L456I |
| 11 | 2 | K48H, S49N, L63A, H65Q, K87H, Y100W, S107K, Q109S, R113N, Y114Q, T117S, |
| | | K134Q, V137A, S197Q, K200H, E204N, T205D, K281A, S286N, A294W, S295N, |
| | | S340G, T381H, E392F, I412F, V427T, S428A, S435R, R448N, L456I |
| 12 | 2 | L63A, H65Q, K87H, Y100W, S107K, Q109S, R113N, T117S, E129H, K134Q, |
| | | V137A, S197Q, E204N, T205D, K281A, S286N, A294W, S295N, S340G, T381H, |
| | | E392F, I412F, V427T, S428A, S435R, R448N, L456I |
| 13 | 2 | K48H, S49N, L63A, K64Q, H65Q, D72E, K87H, Y100W, S107K, Q109S, R113N, |
| | | Y114Q, T117S, A127E, E130K, V133L, K134Q, V137A, W175R, S197Q, K200I, |
| | | F202Y, E204N, T205D, A206C, N208S, F216Y, N229G, T241N, K281E, S286N, |
| | | S291G, A294Q, S295N, S340G, V355T, Q374K, T381H, P390D, E392F, I402T, |
| | | I412F, V427T, S428A, S435R, R448N, L456I |
| 14 | 2 | K48Q, L63A, H65Q, K87H, Y100W, S107K, Q109S, R113N, T117S, E129H, K134Q, |
| | | V137A, S197Q, E204N, T205D, K281A, S286N, A294W, S295N, S340G, T381H, |
| | | E392F, I412F, V427T, S428A, S435R, R448N, L456I |
| 15 | 2 | K48H, S49N, L63A, K64A, H65Q, D72E, K87H, Y100W, S107K, Q109S, R113N, |
| | | Y114Q, T117S, E129H, V133L, K134Q, V137A, S197Q, K200I, E204N, T205D, |
| | | A206C, T241N, K281E, S286N, S291G, A294Q, S295N, S340G, V355T, T381H, |
| | | I402T, Q406R, I412F, V427T, S428A, S435R, R448N, L456I |
| 16 | 2 | K48H, S49N, L63A, H65Q, K87H, Y100W, S107K, Q109S, R113N, Y114Q, T117S, |
| | | K134Q, V137A, S197Q, E204N, T205D, K281A, S286N, S295N, S340G, T381H, |
| | | E392F, I402T, I412F, V427T, S428A, S435R, R448N, L456I |
| 17 | 2 | K48H, L63A, H65Q, K87H, Y100W, S107K, Q109S, R113N, T117S, E129H, K134Q, |
| | | V137A, S197Q, E204N, T205D, K281A, S286N, S295N, S340G, T381H, E392F, |
| | | I412F, V427T, S428A, S435R, R448N, L456I |
| 18 | 2 | V427T, S428A, S435R, R448N, L456I |
| 19 | 2 | K48H, S49N, L63A, H65Q, K87H, Y100W, S107K, Q109S, R113N, Y114Q, T117S, |
| | | K134Q, V137A, S197Q, E204N, T205D, K281A, S286N, A294W, S295N, S340G, |
| | | T381H, E392F, I402T, I412F, V427T, S428A, S435R, R448N, L456I |
| 20 | 2 | K48H, S49N, L63A, H65Q, K87H, Y100W, S107K, Q109S, R113N, Y114Q, T117S, |
| | | K134Q, V137A, S197Q, K200I, E204N, T205D, K281A, S286N, A294W, S295N, |
| | | S340G, V355T, T381H, E392F, I402T, I412F, V427T, S428A, S435R, R448N, L456I |
| 21 | 2 | K48H, S49N, L63A, H65Q, K87H, Y100W, S107K, Q109S, R113N, Y114Q, T117S, |
| | | K134R, V137A, S197Q, K200I, E204N, T205D, K281A, S286N, A294W, S295N, |
| | | S340G, V355T, T381H, I402T, Q406R, I412F, V427T, S428A, S435R, R448N, L456I |
| 22 | 2 | K48H, L63A, H65Q, K87H, Y100W, S107K, Q109S, R113N, T117S, K134Q, V137A, |
| | | S197Q, E204N, T205D, K281A, S286N, A294W, S295N, S340G, T381H, E392F, |
| | | I412F, V427T, S428A, S435R, R448N, L456I |
| 23 | 2 | K48H, S49N, L63A, H65Q, K87H, Y100W, S107K, Q109T, R113N, Y114Q, T117S, |
| | | K134R, V137A, S197Q, K200H, E204N, T205D, K281A, S286N, A294W, S295N, |
| | | S340G, T381H, Q406R, I412F, V427T, S428A, S435R, R448N, L456I |
| 24 | 2 | K48H, S49N, L63A, K64Q, H65Q, D72E, K87H, Y100W, S107K, Q109S, R113N, |
| | | Y114Q, T117S, A127E, E129H, E130K, V133L, K134Q, V137A, W175R, S197Q, |
| | | K200A, F202Y, E204N, T205D, A206C, N208S, F216Y, N229G, T241N, K281E, |
| | | S286N, S291G, A294Q, S295N, S340G, V355T, Q374K, T381H, P390D, I402T, |
| | | Q406R, I412F, V427T, S428A, S435R, R448N, L456I |
| 25 | 3 | Y9H, R26G, G30R, V41K, A64W, N102Y, T127R, I130F, S147T, T157H, V171L, |
| | | I205Q, N231D, L253Q, N266G, K270Q, D277Q, Y286C, I292A, K302G, R328A, |
| | | K331A, R334H, M354L, G368A, P386R, T391Q, R394L, L411M, N421D, D437N, |
| | | P444D, S463P, P473L |
| 26 | 3 | Y9H, G30R, V41K, A64W, N102Y, T127R, I130F, S147T, L167F, V171L, N231D, |
| | | N266G, K270Q, D277Q, Y286C, I292A, K302G, R328A, K331A, R334H, M354L, |
| | | G368A, P386S, T391Q, R394L, L411M, N421D, E425H, D437N, P444D, S463P, |
| | | P473L |
| 27 | 3 | Y9H, R26G, G30R, V41K, A64W, N102Y, I105L, T127R, I130F, S147T, T157H, |
| | | V171L, V177I, I205Q, F215W, N231D, L253Q, K270Q, D277Q, Y286C, I292A, |
| | | K302G, R328A, K331A, R334H, Q352E, M354L, S355C, G368A, P386S, T391Q, |
| | | R394L, L411M, N421D, D437N, P444D, R445H, S463P, P473L |
| 29 | 3 | Y9H, Y13F, A20S, R26G, G30R, A35N, V41A, A64K, A69G, E75Q, L76M, N102Y, |
| | | I105L, D111E, V112E, G113M, H125W, T127R, I130F, T134S, Q140L, F143L, |
| | | S147T, A153Q, Y155F, T157H, K164R, L167F, V171L, V177I, A182C, S189F, |
| | | Q192E, F198Y, I205Q, A213K, F215W, K216M, A221K, V222A, N231D, R242S, |
| | | D248S, L253Q, A262Q, N266D, K270Q, D277S, V282I, Y286C, A291S, I292A, |
| | | K302G, V303T, T325A, R328A, K331A, R334H, A335C, S336V, S351Q, Q352E, |
| | | M354L, S355C, L365A, G368C, P386S, T391Q, R394L, I401L, H409L, L411M, |
| | | F412Y, N421D, Q422E, E425Q, Q427S, V430R, T434E, D437N, H440S, L443N, |
| | | P444D, R445H, Y448K, P456N, I462M, S463P, M466D, I467A, P473L, E482S |
| 30 | 3 | Y9H, A20S, R26G, G30R, A35N, V41A, A64K, A69G, E75Q, L76M, N102Y, I105L, |
| | | D111E, V112E, G113M, H125W, T127R, I130F, T134S, Q140L, S147T, A153Q, |
| | | Y155F, T157H, K164R, L167F, V171L, V177I, A182C, S189F, Q192E, F198Y, |
| | | I205Q, A213K, F215W, K216E, A221K, V222A, N231D, R242S, D248S, L253Q, |
| | | A262Q, N266D, K270Q, D277S, K281A, V282I, Y286C, A291S, I292A, K302G, |
| | | V303T, T325A, R328A, K331A, R334N, A335C, S336V, S351Q, Q352E, M354L, |
| | | S355C, L365A, G368C, P386S, T391Q, R394L, I401L, H409L, L411M, F412Y, |
| | | N421D, Q422E, E425Q, Q427S, V430R, T434E, D437N, H440S, L443N, P444D, |
| | | R445H, Y448K, P456N, I462M, S463P, M466D, I467A, P473L, E482S |
| 31 | 3 | Y9H, Y13F, R26G, G30R, A35N, V41A, A64K, A69Y, L76M, N102Y, I105L, D111E, |
| | | V112E, G113M, T127R, I130F, S147T, Y155F, T157H, K164R, V171L, V177I, |
| | | S189F, F198Y, I205Q, A213K, F215W, K216M, V222A, N231D, R242S, D248S, |
| | | L253Q, A262Q, N266D, K270Q, D277S, K281A, V282I, Y286C, A291S, I292A, |
| | | K302G, T325A, R328A, K331A, R334N, A335C, S336V, S351P, Q352E, M354L, |
| | | S355C, Q362R, L365A, G368C, P386S, T391Q, R394L, H409L, L411M, N421D, |
| | | Q422E, Q427S, V430M, T434E, D437K, H440S, L443N, P444D, R445H, Y448K, |
| | | P456N, I462M, S463P, M466D, I467E, P473L |
| 32 | 3 | Y9H, A20T, G30R, V41K, A64W, N102Y, T127R, I130F, S147T, L167F, V171L, |
| | | N231D, N266G, K270Q, Y286C, I292V, K302G, R328A, K331A, R334H, M354L, |
| | | G368A, P386S, T391Q, R394L, L411M, N421D, D437N, P444D, S463P, P473L |
| 33 | 3 | Y13F, R26G, G30R, A35N, V41A, A64K, A69G, L76M, N102Y, I105L, D111E, |
| | | V112E, G113M, T127R, I130F, S147T, Y155F, T157H, K164R, L167F, V171L, |
| | | V177I, S189F, F198Y, I205Q, A213K, F215W, K216M, V222A, N231D, R242S, |
| | | D248S, L253Q, A262Q, N266D, K270Q, D277S, K281S, V282I, Y286C, A291S, |
| | | I292A, K302G, T325A, R328A, K331A, R334H, A335C, S336V, S351P, Q352E, |
| | | M354L, S355C, Q362R, L365A, G368C, P386R, T391Q, R394L, H409L, L411M, |
| | | N421D, Q422E, E425Q, Q427S, V430W, T434E, D437N, H440S, L443N, P444D, |
| | | R445H, Y448K, P456N, I462M, S463P, M466D, I467E, P473L |
| 34 | 3 | L16M, R26G, G30R, V41A, E42N, A64K, L76M, N102Y, I105L, D111E, T127R, |
| | | I130F, S147T, Y155F, T157H, L167F, V171L, V177I, S189F, I205Q, A213K, F215W, |
| | | K216M, V222A, N231D, R242S, L253Q, N266G, K270Q, K281S, Y286C, I292V, |
| | | K302G, R328A, K331A, R334H, A335C, S336V, S351P, Q352E, M354L, S355C, |
| | | Q362R, L365M, G368A, P386R, T391Q, R394L, H409L, L411M, N421D, Q422E, |
| | | E425H, Q427S, D437N, H440S, L443N, P444D, R445H, Y448K, P456N, I462M, |
| | | S463P, P473L |
| 35 | 3 | R26G, G30R, V41A, A64K, L76M, N102Y, I105L, D111E, T127R, I130F, S147T, |
| | | Y155F, T157R, L167F, V171L, V177I, S189F, I205Q, A213K, F215W, K216M, |
| | | V222A, N231D, R242S, L253Q, N266G, K270Q, D277Q, K281S, Y286C, I292A, |
| | | K302G, R328A, K331A, R334N, A335C, S336T, S351P, Q352E, M354L, S355C, |
| | | Q362R, L365M, G368A, P386S, T391Q, R394L, H409L, L411M, N421D, Q422E, |
| | | E425H, Q427S, D437N, H440S, L443N, P444D, R445H, Y448K, P456N, I462M, |
| | | S463P, P473L |
| 36 | 3 | Y9H, R26G, G30R, V41K, A64W, N102F, I105L, T127R, I130F, S147T, T157H, |
| | | L167F, V171L, V177I, I205Q, F215W, N231D, L253Q, N266G, K270Q, D277Q, |
| | | Y286C, I292A, K302G, R328A, K331A, R334H, Q352E, M354L, S355C, G368A, |
| | | P386S, T391Q, R394L, L411M, N421D, D437N, P444D, R445H, S463P, P473L |
| 37 | 3 | Y9H, R26G, G30R, V41R, A64W, N102Y, I105L, T127R, I130F, S147T, T157H, |
| | | V171L, V177I, I205Q, F215W, V222A, N231D, R242S, L253Q, N266G, K270Q, |
| | | Y286C, I292A, K302G, R328A, K331A, R334H, A335C, S336V, Q352E, M354L, |
| | | S355C, G368A, N374Q, P386S, T391Q, R394L, L411M, N421D, E425H, D437N, |
| | | H440S, P444D, R445H, P456N, S463P, P473L |
| 38 | 3 | Y9H, R26G, G30R, V41K, A64W, N102Y, T127R, I130F, S147T, T157H, V171L, |
| | | I205Q, N231D, L253Q, N266G, K270Q, D277Q, Y286C, I292A, K302G, R328A, |
| | | K331A, R334H, M354L, G368A, P386S, T391Q, R394L, L411M, N421D, E425H, |
| | | D437N, P444D, S463P, P473L |
| 39 | 3 | Y9H, R26G, G30R, V41A, A64W, N102Y, I105L, T127R, I130F, S147T, T157H, |
| | | L167F, V171L, V177I, I205Q, F215W, V222A, N231D, R242S, L253Q, N266G, |
| | | K270Q, D277Q, Y286C, I292A, K302G, R328A, K331A, R334H, A335C, Q352E, |
| | | M354L, S355C, G368A, N374Q, P386S, T391Q, R394L, L411M, N421D, E425H, |
| | | D437N, H440S, P444D, R445H, P456N, S463P, P473L |
| 40 | 4 | I4T, V7S, D39A, K42H, V44L, G47A, G48N, C49V, I60V, A63K, K67M, A73Y, R75H, |
| | | K82R, K87H, I88L, A90V, V105H, F106T, A129S, L137I, V141C, L146V, E161R, |
| | | A173D, D175L, T197N, V201A, V216Q, E249T, K272S, I274E, E282A, L291C, |
| | | R292G, N294S, A296S, K298R, V309R, F319Y, V322T, G324N, P330A, A332L, |
| | | T341I, E357N, Q363E, E364Q, S371N, S372A, G373A, Y377D, D379H, T392A, |
| | | L408R, K439A, S451C, A454V, D486N |
| 41 | 4 | I4T, V7S, K42H, V44L, K45A, G47A, G48N, C49V, I60V, A63K, K67Q, R75H, K87H, |
| | | I88L, A90V, V105F, F106T, A129S, L137I, E161R, A173D, D175L, T197N, V201A, |
| | | V216R, E249T, K272S, E282A, V287I, L291T, N294S, A296S, K298R, V309R, |
| | | V322T, G324N, P330A, T341I, E357N, Q363E, E364Q, S371N, G373A, Y377G, |
| | | T392A, L408R, T431E, K439A, S451N, A454V, D486N |
| 42 | 4 | D486N |
| 43 | 4 | I4T, V7S, G23D, V30N, D39A, K42M, V44L, K45A, G47A, G48N, C49V, I60N, K67M, |
| | | A73G, R75H, L80M, K87H, I88L, A90V, F106T, I113V, Q115E, A129S, L137I, |
| | | V141C, L146V, E161Q, A173D, D175L, E196Q, T197N, V201A, M215L, V216K, |
| | | E221N, V223A, Q225K, M233S, G235K, E249T, N271Q, K272C, I274E, I275Q, |
| | | G277L, E282A, L291C, R292G, N294T, I295L, A296S, K298R, S300A, N307K, |
| | | V309R, F319Y, V322T, G324N, P330A, E357N, Q363E, E364G, S371N, S372A, |
| | | G373A, Y377G, D379H, T392A, L398M, L408R, N409E, T431N, D432Q, K439A, |
| | | P450H, S451C, N453E, A454V, S456D, D486N |
| 44 | 4 | I4T, V7S, D39A, K42Q, V44L, K45A, G47A, G48N, C49V, I60V, K67Q, A73Y, R75H, |
| | | K87H, I88L, A90V, V105F, F106T, A129S, L137I, V141C, E161R, A173D, D175L, |
| | | T197N, V201A, V216Q, E249T, K272S, I274E, E282A, L291C, R292G, N294T, |
| | | A296S, K298R, V309R, F319Y, V322T, G324N, P330A, E357N, Q363E, E364A, |
| | | S371N, S372A, G373A, Y377G, D379H, T392A, L408R, T431E, K439A, S451N, |
| | | A454V, D486N |
| 45 | 4 | I4T, V7S, G23D, V30N, D39W, K42M, V44L, G47A, G48N, C49E, I60V, A63K, |
| | | K67Q, A73G, R75H, K87H, I88L, A90V, V105H, F106T, I113V, Q115E, A129S, |
| | | V141C, L146V, E161R, A173D, D175L, T197N, V201A, V216Q, E221N, G235K, |
| | | E249T, T267Q, K272S, I274E, I275H, Q279R, E282A, L291C, R292G, N294T, |
| | | A296S, K298R, V309R, F319Y, V322T, G324N, P330A, E357N, Q363E, E364Q, |
| | | S371N, S372A, G373A, Y377G, D379H, T392A, L408R, N409E, T431E, D432S, |
| | | K439A, P450H, S451C, N453E, A454V, S456D, D486N |
| 46 | 4 | I4T, V7S, K42H, V44L, K45A, G47A, G48N, C49V, I60V, K67Q, K87H, I88L, A90V, |
| | | V105F, A129S, L137I, L146V, E161R, A173D, D175L, T197N, V201A, V216R, |
| | | E249T, K272C, I275Q, E282A, L291T, N294T, I295L, A296S, K298R, V309R, |
| | | V322T, G324N, P330A, T341I, E357N, Q363E, E364Q, S371N, G373A, Y377D, |
| | | T392Q, L408R, T431E, K439A, S451C, A454V, A458P, D486N |
| 47 | 4 | I4T, V7S, K42H, V44L, K45A, G47A, G48N, C49V, I60V, A63K, K67Q, K87H, I88L, |
| | | A90V, V105C, A129S, L137I, L146V, E161R, A173D, D175L, T197N, V201A, |
| | | V216R, E249T, K272S, E282A, L291T, N294S, A296S, K298R, V309R, V322T, |
| | | G324N, P330A, T341I, E357N, Q363E, E364Q, S371N, G373A, Y377G, T392Q, |
| | | L408K, T431E, K439A, S451N, A454V, D486N |
| 48 | 4 | I4T, V7S, G23D, A27R, V30N, V37C, D39Q, K42M, V44L, K45A, G47A, G48N, |
| | | C49E, I60N, I61V, A63S, K67Q, A73G, R75H, L80M, V81C, K87H, I88L, A90V, |
| | | V105H, F106T, I113V, Q115E, N127D, A129S, V141C, L146V, E161R, A173D, |
| | | D175L, E196Q, T197N, V201A, M215L, V216K, E221N, V223A, Q225K, M233L, |
| | | G235K, E249T, R252K, N271Q, K272S, I274E, I275Q, G277L, V280I, E282A, |
| | | L291C, R292G, N294T, I295L, A296S, K298R, S300A, N307K, H308T, V309R, |
| | | F319Y, V322T, A323R, G324N, P330A, A332L, E357N, Q363E, E364Q, S371N, |
| | | S372A, G373A, K375D, Y377G, D379H, T392A, L408R, N409E, Q430E, T431N, |
| | | D432N, K439A, K442D, P450H, S451C, N453E, A454V, S456D, N464K, D486N |
| 49 | 4 | I4T, V7S, K42E, V44L, G47A, G48N, C49V, M53V, I60V, K67Q, R75K, K87H, I88L, |
| | | A90V, V105F, F106T, A129S, L137I, E161R, D175L, T197N, V201A, V216R, E249T, |
| | | D260E, T267Q, K272S, E282A, L291T, N294T, A296S, K298R, V309R, V322T, |
| | | G324N, P330A, E357N, Q363E, E364Q, S371N, G373A, Y377G, T392A, L408R, |
| | | T431E, K439A, S451C, A454V, D486N |
| 50 | 4 | I4T, V7S, G23D, V30N, D39W, K42M, V44L, K45A, G47A, G48N, C49E, I60N, |
| | | A63K, K67Q, A73Y, R75H, L80M, K87H, I88L, A90V, F106T, I113V, Q115E, A129S, |
| | | L137I, V141C, L146V, E161Q, A173D, D175L, E196Q, T197N, V201A, M215L, |
| | | V216K, E221N, V223A, Q225K, M233S, G235K, E249T, K272V, I274E, G277L, |
| | | E282A, L291C, R292G, N294T, A296S, K298R, S300A, N307K, V309R, F319Y, |
| | | V322T, G324N, P330A, E357N, Q363E, E364G, S371N, S372A, G373A, Y377G, |
| | | D379H, T392A, L398M, L408K, N409E, T431N, D432Q, F434Y, K439A, P450H, |
| | | S451C, N453E, A454V, S456D, D486N |
| 51 | 4 | I4T, V7S, V30N, D39A, K42Q, V44L, G47A, G48N, C49V, I60V, A63K, K67Q, A73Y, |
| | | I88L, A90V, V105F, F106T, I113V, A129S, L137I, V141C, L146V, D165N, A173D, |
| | | D175L, T197N, V201S, V216K, E221N, E249T, K272S, I274E, E282A, L291C, |
| | | R292G, N294T, A296S, K298R, V309R, F319Y, V322T, G324N, P330A, E357N, |
| | | Q363E, E364Q, S371N, S372A, G373A, Y377G, D379H, T392A, L408R, T431E, |
| | | F434Y, K439A, P450H, S451C, A454V, S456D, D486N |
| 52 | 4 | I4T, V7S, V30N, D39W, K42M, V44L, G47A, G48N, C49V, I60V, K67Q, A73Y, |
| | | R75H, I88L, A90V, V105C, F106T, I113V, A129S, V141C, L146V, E161R, A173D, |
| | | D175L, T197N, V201A, V216K, E221N, E249T, K272S, I274E, E282A, L291C, |
| | | R292G, N294S, A296S, K298R, V309R, F319Y, V322T, G324N, P330A, T341I, |
| | | E357N, Q363E, E364Q, S371N, S372A, G373A, Y377D, D379H, T392A, L408R, |
| | | K439N, P450H, S451C, A454V, S456D, D486N |
| 53 | 4 | I4T, V7S, G23D, A27R, V30N, V37C, D39Q, K42M, V44L, G47A, G48N, C49E, I60N, |
| | | I61V, A63S, K67Q, A73Y, L80M, V81C, K87H, I88L, A90V, V105H, F106S, I113V, |
| | | Q115E, N127D, A129S, L137I, V141C, L146V, E161R, D175L, E196Q, T197N, |
| | | V201A, M215L, V216K, E221N, V223A, Q225K, M233L, G235K, E249T, R252K, |
| | | N271Q, K272S, I274E, I275Q, G277L, V280I, E282A, L291C, R292G, N294A, I295L, |
| | | A296S, K298R, S300A, N307K, H308T, V309R, F319Y, V322T, A323R, G324N, |
| | | P330A, A332L, T341I, E357N, Q363E, E364G, S371N, S372A, G373A, K375D, |
| | | Y377G, D379H, T392A, L398M, L408R, N409E, Q430E, T431N, D432Q, L436I, |
| | | K439A, K442D, P450H, S451C, N453E, A454V, S456D, N464K, D486N |
| 54 | 4 | I4T, V7S, G23D, V30N, D39W, K42Q, V44L, K45A, G47N, G48N, C49E, I60V, |
| | | K67R, A73Y, K87H, I88L, A90V, V105F, F106T, I113V, Q115E, A129S, L137I, |
| | | V141C, L146V, E161Q, A173D, D175L, T197N, V201A, V216K, E221N, G235K, |
| | | E249T, K272S, I274E, E282A, L291C, R292K, N294S, A296S, K298R, V309R, |
| | | F319Y, V322T, G324N, P330A, T341I, E357N, Q363E, E364A, S371N, S372A, |
| | | G373A, Y377D, D379H, T392A, L408R, N409E, T431E, D432Q, K439A, P450H, |
| | | S451C, N453E, A454V, S456D, D486N |
| 55 | 5 | L81C, P83Y, D84G, E85N, A86D, T87Y, G89S, K90Q, N91C |
| 57 | 6 | D34S, D64G, R70C, I84V, D141S, L152A, L154A, I155K, D160I, R171A, G177S, |
| | | K239E, Q260H, Y261L, D277S, E303R, E316A, D323K, K329S, L335R, A336Q, |
| | | E339Q, I345A, D358T, S365N, D397G, G412T, A421K, T436G, P471S, N531H, |
| | | I542T, K578Q, A585T, T589Y, K594T, T599G, K605E, S606P, A638V, T643N, |
| | | P646T, S663T, V706T, D712T |
| 58 | 6 | D34S, K36T, D64G, R70C, I84V, D86N, K115N, D141S, D150N, L152A, L154A, |
| | | I155K, T156N, S157L, D160V, R161M, V168T, R171A, G177S, E180D, L191M, |
| | | F238L, K239E, F255H, Q260H, Y261L, G275S, D277S, E303R, E316A, R322A, |
| | | D323K, T325N, K329W, V334M, L335R, A336Q, S337K, E339A, Q341D, D343N, |
| | | Y344W, I345A, M348L, D358S, V362Y, G364N, S365G, G366S, A369K, T370Y, |
| | | E371H, D397G, G412T, V418I, A421K, T436G, R440V, N443T, S467T, A468T, |
| | | A469S, P471S, S473T, S477T, S526T, N531H, I542T, A554K, G568N, K578Q, |
| | | A585T, A586T, T589A, K594T, T599G, A600T, F604Y, K605V, S606P, T630E, |
| | | A638V, A639S, T643N, P646T, Y654F, I658M, S663N, I681L, T690V, V706T, D712T |
| 59 | 6 | D34S, D64G, R70C, I84V, D141S, L152A, L154A, I155K, D160I, R171A, G177S, |
| | | F238L, K239E, Q260H, Y261L, D277S, E303R, E316A, D323K, K329S, L335R, |
| | | A336Q, E339Q, D343N, I345A, D358T, S365N, D397G, G412T, A421K, T436G, |
| | | P471S, N531H, I542T, K578Q, A585T, T589Y, K594T, T599G, K605E, S606P, |
| | | A638V, T643N, P646T, S663T, V706T, D712T |
| 60 | 6 | D34S, D64G, R70C, I84V, D141S, L152A, D160I, R161M, G177S, F238L, Q260H, |
| | | Y261L, D277S, E303R, E316K, D323K, K329F, L335R, A336L, D397G, A421K, |
| | | T436G, T438W, N443T, P471S, I542T, A585T, K594T, T599G, K605E, T643N, |
| | | P646T, V706T, D712T |
| 61 | 6 | D34S, D64G, R70C, I84V, D141S, L152A, D160I, R161M, G177S, F238L, Q260H, |
| | | Y261L, D277S, E303R, E316K, D323K, K329F, L335R, A336Q, D397G, A421K, |
| | | T436G, T438W, N443T, P471S, I542T, K578T, A585T, K594Q, T599G, N602S, |
| | | K605E, T643N, P646T, V706T, D712T |
| 62 | 6 | D34S, D64G, R70C, I84V, D141S, L152A, I155K, D160I, R161M, R171A, G177S, |
| | | F238L, Q260H, Y261L, D277S, E303R, E316A, D323K, K329S, L335R, A336Q, |
| | | E339Q, I345A, S365N, D397G, A421K, T436G, N443T, P471S, N531H, I542T, |
| | | A585T, K594T, T599G, K605E, A638V, T643N, P646T, V706T, D712T |
| 63 | 6 | D34N, K36T, D56N, D64G, R70C, I84V, D86N, K115N, Q134K, D141S, D150N, |
| | | L152A, L154A, I155K, T156N, S157L, D160I, R161M, V168T, R171A, N175D, |
| | | G177S, E180D, D186N, S189N, L191M, L201Y, F238L, K239E, L244W, V250F, |
| | | F255H, Q260H, Y261L, G264P, S268T, G275S, D277S, Y287F, E303R, E316A, |
| | | R322A, D323K, T325N, K329W, V334M, L335R, A336L, S337K, E339A, Q341D, |
| | | D343N, Y344W, I345A, M348L, D358S, V362Y, G364N, S365G, G366S, A369K, |
| | | T370Y, E371H, D397G, G412T, V418I, A421K, S428L, T436G, T438W, E439Q, |
| | | R440V, N443T, S455G, S467T, A468T, A469T, P471S, S473T, S477T, N507T, |
| | | S526T, N531H, I542T, A554K, G568N, K578Q, I581V, A585T, A586T, T589A, |
| | | K594T, T599G, A600T, N602S, F604Y, K605V, S606P, L620V, T630Q, A638V, |
| | | A639S, T643N, P646T, Y654F, S663T, K675Q, I681V, K684R, T690V, A703T, |
| | | V706T, D712T |
| 64 | 6 | D34S, D64G, R70C, I84V, D141S, L152A, I155K, D160I, R161M, R171A, G177S, |
| | | F238L, Q260H, Y261L, D277S, E303R, E316A, D323K, K329S, L335R, A336L, |
| | | E339Q, I345A, S365N, D397G, A421K, T436G, T438W, P471S, N531H, I542T, |
| | | A585T, K594T, T599G, K605V, A638V, T643N, P646T, V706T, D712T |
| 66 | 6 | D34S, D64G, R70C, I84V, K115N, D141S, L152A, L154A, I155K, D160I, R171A, |
| | | G177S, E180D, L191M, F238L, K239E, Q260H, Y261L, D277S, E303R, E316A, |
| | | F321I, D323K, K329S, V334M, L335R, E339Q, I345A, D358T, V362Y, S365G, |
| | | G366S, T370Y, E371H, D397G, G412T, A421K, T436G, A469T, P471S, S477T, |
| | | N531H, I542T, K578Q, A585T, T589Y, K594T, T599G, F604Y, K605V, S606P, |
| | | T630E, A638T, T643N, P646T, S663T, V706T, D712T |
| 67 | 6 | D34S, K36T, D64G, R70C, I84V, D86N, K115N, D141S, D150N, L152A, L154A, |
| | | I155K, T156N, S157L, D160I, R161M, V168T, R171A, G177S, E180D, L191M, |
| | | K239E, L244W, F255H, Q260H, Y261L, G275S, D277S, E303R, Y313L, E316A, |
| | | R322A, D323K, T325N, K329S, V334M, L335I, A336Q, S337R, E339A, Q341D, |
| | | D343N, Y344W, I345A, M348L, D358S, V362Y, G364N, S365G, G366S, A369K, |
| | | T370Y, E371H, D397G, G412T, V418I, A421K, T436G, S467T, A468T, A469T, |
| | | P471S, S473T, S477T, S526T, N531H, I542T, A554K, G568N, K578Q, A585T, |
| | | A586T, T589A, K594T, T599G, A600Q, F604Y, K605V, S606P, T630E, A638V, |
| | | A639S, T643N, P646T, Y654F, S663N, I681L, T690V, V706T, D712T |
| 68 | 6 | D34S, K36T, D64G, R70C, I84V, D86N, K115N, D141S, D150N, L152A, L154A, |
| | | I155K, T156N, S157L, D160I, R161M, V168T, R171A, G177S, E180D, L191M, |
| | | K239E, F255H, Q260H, Y261L, G275S, D277S, E303R, E316A, R322A, D323K, |
| | | T325N, K329W, V334M, L335R, A336Q, S337R, E339A, D343N, Y344W, I345A, |
| | | D358S, V362Y, G364N, S365G, G366S, T370Y, E371H, D397G, G412T, V418I, |
| | | A421K, T436G, R440V, A468T, A469T, P471S, S473T, S477T, N531H, I542T, |
| | | A554K, G568N, K578T, A585T, T589A, K594Q, T599G, A600T, N602S, F604Y, |
| | | K605V, S606P, T630E, A638V, A639S, T643N, P646T, Y654F, S663N, I681L, |
| | | V706T, D712T |
| 69 | 6 | D34S, D64G, R70C, I84V, K115N, D141S, L152A, L154A, I155K, D160I, R171A, |
| | | G177S, E180D, L191M, K239E, Q260H, Y261L, D277S, E303R, E316A, F321I, |
| | | D323K, T325H, K329S, V334M, L335R, A336Q, D343N, I345A, D358T, V362Y, |
| | | S365G, G366S, T370Y, E371H, D397G, G412T, A421K, T436G, A469T, P471S, |
| | | S477T, N531H, I542T, K578Q, A585T, T589Y, K594T, T599G, F604Y, K605V, |
| | | S606P, T630E, A638T, T643N, P646T, S663T, V706T, D712T |
| 70 | 6 | D34S, K36T, D64G, R70C, I84V, D86N, K115N, D141S, D150N, L152A, L154A, |
| | | I155K, T156N, S157L, D160I, V168T, R171A, G177S, E180D, L191M, F238L, |
| | | K239E, F255H, Q260H, Y261L, G275S, D277S, E303R, E316A, R322A, D323K, |
| | | T325N, K329W, V334M, L335R, A336Q, S337R, E339Q, D343N, Y344W, I345A, |
| | | D358S, V362Y, G364N, S365G, G366S, T370Y, E371H, D397G, G412T, V418I, |
| | | A421K, T436G, R440V, A468T, A469T, P471S, S473T, S477T, N531H, I542T, |
| | | A554K, G568N, K578Q, A585T, T589A, K594T, T599G, A600T, F604Y, K605V, |
| | | S606P, T630E, A638V, A639S, T643N, P646T, Y654F, S663N, V706T, D712T |
| 71 | 6 | D34N, K36T, D56N, D64G, R70C, I84V, D86N, K115N, Q134K, D141S, D150N, |
| | | L152A, L154A, I155K, T156N, S157L, D160I, R161M, V168T, R171A, N175D, |
| | | G177S, E180D, D186N, S189N, L191M, L201Y, K239E, V250F, F255H, Q260H, |
| | | Y261L, G264P, S268T, G275S, D277S, Y287F, E303R, E316A, R322A, D323K, |
| | | T325N, K329S, V334M, L335R, A336L, S337R, E339Q, Q341D, Y344W, I345A, |
| | | M348L, D358S, V362Y, G364N, S365G, G366S, A369K, T370Y, E371H, D397G, |
| | | G412T, V418I, A421K, S428L, T436G, T438W, E439Q, V442M, S455G, S467T, |
| | | A468T, A469T, P471S, S473T, S477T, N507T, S526T, N531H, I542T, A554K, |
| | | G568N, K578Q, I581V, A585T, A586T, T589A, K594T, T599G, A600T, F604Y, |
| | | K605V, S606P, L620V, T630Q, A638V, A639S, T643N, P646T, Y654F, S663T, |
| | | K675Q, I681V, K684R, T690V, A703T, V706T, D712T |
| 72 | 7 | I44V, T72C, G79C, I161M, D186N, N212T, F222Y, Q232N, F233T, K239L, I242A, |
| | | M255H, F263H, M270Q, D271H, D275N, P278N, H297Y, E302N, N322D, D326N, |
| | | N331H, A340D, E343W, M360H, A361S, D362L, E363N, P366N, K368R, V369L, |
| | | V375F, V382T, N396G, M404D, R413T, Q419R, S423A, D435S, S470T, T484K, |
| | | Q486V, A489G, G499N, A503N, G510Q, G515S, A522T, V525H, M534T, K541Q, |
| | | V553K, V562Q, Q590V, Q595G, V596E, A598N, W624S, L649M, V662Y, G669D, |
| | | K670T, K678I, K679R, G681D, I687T, S701N, T705K, V706I, N707I, N709D |
| 73 | 7 | T72C, G79C, I161A, D186N, N212T, F233T, F263Q, M270Q, D271H, D275N, |
| | | P278N, H297Y, E302N, D326T, A340D, E343W, M360L, L373M, V375F, V382T, |
| | | M404D, R413T, D435S, S470T, T484E, A503N, G510Q, A522T, V525H, K541Q, |
| | | Q590V, Q595G, A598N, L649M, G681D, I687T, S701N, T705K, V706I, N707I, |
| | | N709D |
| 74 | 7 | T72C, G79C, I161A, D186N, N212T, F233T, I242A, F263Q, S268E, M270Q, D271N, |
| | | D275N, P278G, H297Y, E302N, D326T, A340D, E343W, M360L, P366N, K368R, |
| | | V369L, V375F, V382T, M404D, R413T, Q419R, S423A, D435S, E449K, S470T, |
| | | A489G, G499N, A503N, G510Q, G515S, A522T, V525H, K541Q, V553K, V562Q, |
| | | Q590V, Q595G, A598N, L649M, K678I, G681D, I687T, S701N, T705K, V706I, |
| | | N707I, N709D |
| 75 | 7 | T72C, G79C, I161A, D186N, F233T, F263Q, M270Q, D271N, D275N, P278N, |
| | | H297Y, E302N, D326T, A340D, E343W, M360L, V375F, V382T, M404D, R413T, |
| | | D435S, S470T, T484E, A503N, G510Q, A522T, V525H, K541Q, Q590V, A598N, |
| | | L649M, G681D, I687T, S701N, T705K, V706I, N707I, N709D |
| 76 | 7 | T72C, G79C, I161A, D186N, N212T, F222Y, Q232N, F233T, K239L, I242A, F263H, |
| | | S268E, M270Q, D271N, D275N, P278N, H297Y, E302N, D326N, A340D, E343W, |
| | | M360H, E363N, P366N, K368R, V369L, V375F, V382T, M404D, R413T, Q419R, |
| | | S423A, D435S, S470T, Q486V, A489G, G499N, A503N, G510Q, G515S, A522T, |
| | | V525H, M534T, K541Q, V553K, V562Q, Q590V, Q595G, V596E, A598N, L649M, |
| | | V662Y, K678I, K679R, G681D, I687T, S701N, T705K, V706I, N707I, N709D |
| 77 | 7 | I44V, T72C, G79C, I161M, D186N, S200G, N212T, F222Y, Q232N, F233T, K239I, |
| | | I242A, M255H, F263Q, S268K, M270Q, D271N, D275N, P278N, H297Y, E302N, |
| | | L316V, V319A, N322D, D326T, N331H, A340D, D342N, E343W, M360L, A361S, |
| | | D362L, E363N, P366N, K368R, V369L, V375F, V382T, N396G, M404D, R413T, |
| | | Q419R, S423A, D435S, T436Q, L465S, S470T, T484E, A489G, G499N, A503N, |
| | | G510Q, G515S, A522T, V525H, M534S, K541Q, V553K, V562Q, Q590V, Q595G, |
| | | V596E, A598N, W624S, S643D, L649M, V662Y, E668A, K670T, K678I, G681D, |
| | | I687T, S701N, T705K, N707I, N709D |
| 78 | 7 | I44V, S65A, E96D, A113S, D119S, T122A, T135P, I191L, T198A, S200M, N212S, |
| | | F227L, Q232P, F233V, K236R, I242V, L244M, L246I, F269L, V273I, Y274D, D275N, |
| | | S294A, F298Y, D326N, I345L, A361I, E363G, I371M, T406S, S427N, S432A, |
| | | S443G, I447V, L465S, K467S, S468N, S480A, A489G, A503S, N508D, T518A, |
| | | A522T, V525T, I531V, K541Q, L542V, V553T, N560T, V562A, S564N, T574V, |
| | | T576A, I580V, P581S, A582P, T591S, G593S, V596T, N621T, W624L, E626Q, |
| | | L630I, H635Y, N641D, L649M, I654V, V662I, G681S, S682Q, I686T, S700N, T704K, |
| | | V705I, N706I, N708D |
| 79 | 7 | I44V, T72C, G79C, I161M, D186N, S200G, N212T, F222Y, Q232N, F233T, K239I, |
| | | I242A, L244W, M255H, F263H, D271H, D275N, P278N, S288G, E289G, N295D, |
| | | H297Y, E302N, K315A, Q318E, V319S, N322D, S324N, D326T, N331H, T337Q, |
| | | A338Q, A340D, D342N, E343W, M360L, A361S, D362L, E363N, P366N, K368R, |
| | | V369L, V375F, V382T, N396G, M404D, R413T, Q419R, S423A, D435S, T436Q, |
| | | I447V, L465S, S470T, T484K, A489G, G499N, N501D, A503N, G510Q, G515A, |
| | | A522T, E523T, V525H, M534S, H540N, K541Q, T551D, V553K, V562Q, Q572E, |
| | | T576S, Q590V, T591V, Q595G, V596K, A598N, A599N, D601V, W624S, S643D, |
| | | L649M, V662Y, E668A, K670T, K675M, K678I, K679R, G681D, I687T, S690N, |
| | | S701N, T705K, N707I, N709D |
| 80 | 7 | T72C, G79C, I161A, D186N, N212T, F233T, F263Q, S268E, D271N, D275N, |
| | | P278N, H297Y, E302N, D326T, N331Y, A340D, E343W, M360L, P366N, K368R, |
| | | V369L, V375F, V382T, M404D, R413T, Q419R, S423A, D435S, E449K, S470T, |
| | | T484E, A489G, G499N, A503N, G510Q, G515S, A522T, V525H, K541Q, V553K, |
| | | V562Q, Q590V, Q595G, A598N, L649M, K678I, G681D, I687T, S701N, T705K, |
| | | V706I, N707I, N709D |
| 81 | 7 | T72C, G79C, I161C, D186N, N212T, F222Y, Q232N, F233T, K239L, I242A, F263H, |
| | | S268E, M270Q, D271N, D275N, P278N, H297Y, E302N, N322D, D326N, A340D, |
| | | E343W, M360L, E363N, P366N, K368R, V369L, V375F, V382T, M404D, R413T, |
| | | Q419R, S423A, D435S, S470T, T484K, A489G, G499N, A503N, G510Q, G515S, |
| | | A522T, V525H, M534T, K541Q, V553K, V562Q, Q590V, Q595G, V596E, A598N, |
| | | L649M, V662Y, K678I, K679R, G681D, I687T, S701N, T705K, V706I, N707I, N709D |
| 82 | 7 | T72C, G79C, I161C, D186N, F233T, I242A, F263Q, M270Q, D271N, D275N, |
| | | P278N, H297Y, E302N, D326T, A340D, E343W, M360L, L373M, V375F, V382T, |
| | | M404D, R413T, D435S, S470T, T484E, A503N, G510Q, A522T, V525H, K541Q, |
| | | Q590V, A598N, L649M, G681D, I687T, S701N, T705K, V706I, N707I, N709D |
| 83 | 7 | T72C, G79C, I161A, D186N, N212T, F233T, I242A, F263Q, M270Q, D271H, D275N, |
| | | P278N, H297Y, E302N, D326T, A340D, E343W, M360L, L373M, V375F, V382T, |
| | | M404D, R413T, D435S, S470T, T484E, A503N, G510Q, A522T, V525H, K541Q, |
| | | Q590V, Q595G, A598N, L649M, G681D, I687T, S701N, T705K, V706I, N707I, |
| | | N709D |
| 84 | 7 | I44V, T72C, G79C, I161M, D186N, S200G, N212T, F222Y, Q232N, F233T, K239I, |
| | | I242A, M255H, F263Q, S268K, D271N, D275N, P278N, H297Y, E302N, L316I, |
| | | V319A, D326T, N331H, A340D, D342N, E343W, M360H, A361S, D362L, E363N, |
| | | P366N, K368R, V369L, V375F, N396G, M404D, R413T, Q419R, S423A, D435S, |
| | | T436Q, L465S, S470T, T484E, A489G, G499N, A503N, G510Q, G515S, A522T, |
| | | V525H, M534S, K541Q, V553K, V562Q, Q590V, Q595G, V596K, A598N, W624S, |
| | | S643D, L649M, V662Y, E668A, G669D, K670T, K678I, K679R, G681D, I687T, |
| | | S701N, T705K, N707I, N709D |
| 85 | 7 | I44V, T72C, G79C, I161M, D186N, N212T, F222Y, Q232N, F233T, K239L, I242A, |
| | | M255H, K259R, F263H, S268K, M270Q, D271H, D275N, P278G, H297Y, E302N, |
| | | L316V, D326N, N331H, A340D, E343W, M360H, A361S, D362L, E363N, P366N, |
| | | K368N, V369L, V375F, V382T, N396G, M404D, R413T, Q419R, S423A, D435S, |
| | | S470T, T484E, A489G, G499N, A503N, G510Q, G515S, A522T, V525H, M534T, |
| | | K541Q, V553K, V562Q, Q590V, Q595G, V596E, A598N, W624S, L649M, V662Y, |
| | | G669D, K670T, K678I, K679R, G681D, I687T, S701N, T705K, V706I, N707I, N709D |
| 86 | 7 | T72C, G79C, G681D, I687T, S701N, T705K, V706I, N707I, N709D |
| 87 | 7 | I44V, T72C, G79C, E96D, I161M, D186N, S200G, N212T, F222Y, Q232N, F233T, |
| | | K239L, I242A, L244W, M255H, F263H, S268K, M270Q, D271N, D275N, P278N, |
| | | S288G, E289G, N295D, H297Y, E302N, K315A, Q318E, V319A, N322D, S324N, |
| | | D326T, N331H, T337Q, A340D, D342N, E343W, M360L, A361S, D362L, E363N, |
| | | P366N, K368R, V369L, V375F, V382T, N396G, M404D, N411T, R413T, Q419R, |
| | | S423A, D435S, T436Q, R439L, I447V, L465S, S470T, T484E, A489G, G499N, |
| | | N501D, A503N, G510Q, G515A, E523T, V525H, M534S, H540N, K541Q, T551D, |
| | | V553K, V562Q, Q572E, T576S, Q590V, T591V, Q595G, V596K, A598N, A599N, |
| | | D601V, W624S, S643D, L649M, V662Y, E668A, G669D, K670T, K675M, K678I, |
| | | K679R, G681D, I687T, S690N, S701N, T705K, N707I, N709D |
| 88 | 8 | L14M, A15Q, V22I, H24E, P27K, F28Y, S29T, V30S, N31Q, S34C, N36D, G42A, |
| | | E43K, I44V, D46K, S47N, I53M, L54C, E55T, P56T, M60C, S80T, A83L, S87D, |
| | | R88N, V99M, R111N, I114M, K116N, G118A, L135M, H142E, N143E, E146G, |
| | | Y147R, V151F, E153R, I156A, K158R, P160L, H161T, H163N, F167Y, T173H, |
| | | E186S, F189N, E199D, L202F, K203Q, S209V, T211C, L216R, I217M, L219A, |
| | | I220Y, H223N, R231G, K241Q, K244L, L248M, V265L, S282H, E286Q, Q287A, |
| | | H317Q, I318M, E323G |
| 89 | 8 | A15Q, P27K, S34C, G42A, D46K, S47N, E55T, P56T, A83L, R111N, K116N, |
| | | N143E, Y147R, V151F, H163N, F189I, S209V, T211C, L216R, L219A, H223N, |
| | | R231Q, K241Q, K244L, L248M, S282H, E286Q, Q287A, I318M |
| 90 | 8 | I318M |
| 91 | 8 | H24G |
| 92 | 8 | Q19H, H24Y, S29A, V30A, K33Q, I53V, S59T, E65D, P69S, L72M, H115N, I130L, |
| | | V132I, L135I, T140S, H142Y, N143E, Y147F, F149Y, F152M, K154T, H161R, |
| | | Q172E, D193E, R231H, H290Q, N291S, I318M |
| 93 | 8 | A15Q, H24E, P27K, F28Y, S29A, N31Q, S34C, G42A, E43K, D46K, S47N, L54C, |
| | | E55T, P56T, M60C, H62G, S80T, A83L, R111N, K116N, F121C, L135M, P137R, |
| | | N143E, E146G, Y147R, V151F, P160L, H161T, H163N, E186S, F189M, L202F, |
| | | K203Q, S209V, T211C, L216R, I217M, L219A, H223N, R231G, K241Q, K244L, |
| | | L248M, V265L, S282H, E286Q, Q287A, I318M, E323D |
| 94 | 8 | A15Q, P27K, S34C, G42A, D46K, S47N, E55T, P56T, H62G, A83L, R111N, K116N, |
| | | P137R, N143E, Y147R, V151F, H163N, F189M, S209V, T211C, L216R, L219A, |
| | | H223N, R231G, K241Q, K244L, L248M, S282H, E286Q, Q287A, I318M |
| 95 | 8 | P27K, S34C, D46K, S47N, E55Y, H62G, A83L, R111N, K116N, P137R, N143E, |
| | | Y147R, V151F, H163N, F189M, S209V, T211C, L216K, L219A, R231Q, K241Q, |
| | | L248M, S282H, E286Q, Q287A, I318M |
| 96 | 8 | A15Q, H24E, P27K, F28Y, S29A, N31Q, S34C, G42A, E43K, D46K, S47N, L54C, |
| | | E55T, P56T, M60C, H62G, S80T, A83L, R111N, K116N, L135M, P137R, N143E, |
| | | E146N, Y147R, V151F, P160L, H161T, H163N, E186S, F189M, L202F, K203Q, |
| | | S209V, T211C, L216R, I217M, L219A, H223N, R231G, K241Q, K244L, L248M, |
| | | V265L, S282H, E286Q, Q287A, I318M, E323D |
| 97 | 8 | L14M, A15Q, H24E, P27K, F28Y, S29A, V30S, N31Q, S34C, N36D, G42A, E43K, |
| | | D46K, S47N, I53M, L54C, E55T, P56T, M60C, S80T, A83L, R88N, V99M, R111N, |
| | | I114M, K116N, G118A, L135M, H142E, N143E, E146G, Y147R, V151F, K158R, |
| | | P160L, H161T, H163N, F167Y, T173H, E186S, F189I, L202F, K203Q, S209V, |
| | | T211C, L216R, I217M, L219A, I220Y, H223N, R231G, K241Q, K244L, L248M, |
| | | V265L, S282H, E286A, Q287A, H317Q, I318M, E323D |
| 98 | 8 | A15Q, P27K, F28Y, S29T, S34C, G42A, D46K, S47N, E55T, P56K, M60C, H62G, |
| | | A83L, R111N, K116N, P137Q, N143E, Y147R, V151F, H161T, H163N, F189M, |
| | | L202F, S209V, T211C, L216R, I217M, L219A, H223N, R231Q, K241Q, K244L, |
| | | L248M, V265L, S282H, E286Q, Q287A, I318M, E323D |
| 100 | 8 | A15Q, P27K, F28Y, S29T, S34C, G42A, D46K, S47N, E55T, P56K, M60C, A83L, |
| | | R111N, K116N, P137Q, N143E, Y147R, V151F, P160L, H161T, H163N, F189I, |
| | | L202F, S209V, T211C, L216R, I217M, L219A, H223N, R231G, K241Q, K244L, |
| | | L248M, V265L, S282H, E286Q, Q287A, I318M, E323D |
| 101 | 8 | A15Q, P27K, S29T, S34C, G42A, D46K, S47N, E55T, P56T, M60C, H62G, A83L, |
| | | R111N, K116N, P137R, N143E, Y147R, V151F, H163N, F189M, L202F, S209V, |
| | | T211C, L216R, L219A, H223N, R231G, K241Q, K244L, L248M, S282H, E286Q, |
| | | Q287S, I318M, E323D |
| 102 | 8 | P27K, S34C, D46K, S47N, E55Y, H62G, A83L, R111N, K116N, N143E, Y147R, |
| | | V151F, H163N, F189M, S209V, T211C, L216K, L219A, R231Q, K241Q, L248M, |
| | | S282H, E286Q, Q287A, I318M |
| 103 | 8 | L14M, A15Q, H24E, P27K, F28Y, S29T, V30S, N31Q, S34V, N36D, G42A, E43K, |
| | | D46K, S47N, I53M, L54C, E55T, P56T, M60C, H62G, S80T, A83L, R88N, V99M, |
| | | R111N, K116N, L135M, P137R, H142E, N143E, E146G, Y147R, V151F, K158R, |
| | | P160L, H161T, H163N, F167Y, T173H, E186S, F189M, L202F, K203Q, S209V, |
| | | T211C, L216R, I217M, L219A, I220Y, H223N, R231G, K241Q, K244L, L248M, |
| | | V265L, S282H, E286A, Q287S, H317Q, I318M, E323D |
| 104 | 8 | A15Q, P27K, S29A, S34C, G42A, D46K, S47N, E55T, P56T, M60C, H62G, A83L, |
| | | R111N, K116N, N143E, Y147R, V151F, H163N, F189I, L202F, S209V, T211C, |
| | | L216R, L219A, H223N, R231G, K241Q, K244L, L248M, S282H, E286Q, Q287A, |
| | | I318M, E323D |
| 105 | 8 | L14M, A15Q, V22I, H24E, P27K, F28Y, S29A, V30K, N31E, S34C, N36D, G42A, |
| | | E43K, I44V, D46K, S47N, I53M, L54C, E55T, P56T, M60C, H62G, S80T, A83L, |
| | | S87D, R88N, V99M, R111N, I114M, K116N, G118A, L135M, P137Q, H142E, |
| | | N143E, E146G, Y147R, V151F, E153R, I156A, K158R, P160L, H161T, H163N, |
| | | F167Y, T173H, E186S, F189N, E199D, L202F, K203Q, S209V, T211C, L216R, |
| | | I217M, L219A, I220Y, H223N, R231G, K241Q, K244L, L248M, V265L, S282H, |
| | | E286Q, Q287A, H317Q, I318M, E323G |
| 106 | 9 | F339S, A347D, E350P, Q353G, M354I, V355A, L357V, F362H, K364R, Q365P, |
| | | E375G, D377E, F381Q, T382V, G384E, T385K, R405K, D409K, K410R, K411P, |
| | | I412L, S415G |
| 107 | 9 | F339S, D340E, A347D, E350P, Q353G, M354I, V355A, L357V, F362H, K364R, |
| | | Q365P, E375G, D377E, F381Q, T382V, G384E, T385K, R405K, D409K, K410R, |
| | | K411P, I412L, S415Y |
| 108 | 9 | F339S, D340E, A347D, E350P, Q353G, M354I, V355A, L357V, F362H, K364R, |
| | | Q365P, E375G, D377E, F381Q, T382V, G384E, T385K, R405K, D409K, K410R, |
| | | K411P, I412L, S415G |
| 109 | 9 | F339S, D340E, A347G, E350P, Q353G, M354I, V355A, L357V, F362H, K364R, |
| | | Q365P, E375G, D377E, F381Q, T382V, G384E, T385K, R405K, D409K, K410R, |
| | | K411P, I412L, Y413F, S415G |
| 110 | 9 | F339S, D340E, A347G, E350P, Q353G, M354I, V355A, L357V, F362H, K364R, |
| | | Q365P, E375G, D377E, F381Q, T382V, G384E, T385K, R405K, D409K, K410R, |
| | | K411P, I412L, Y413F, S415Y |
| 111 | 9 | F339R, D340E, A347G, E350P, Q353G, M354I, V355A, L357V, F362H, K364R, |
| | | Q365P, D377E, F381Q, T382V, G384E, T385K, R405K, D409K, K410R, K411P, |
| | | I412L, Y413F, S415Y |
| 112 | 9 | F339S, A347D, E350P, Q353G, M354I, V355A, L357V, F362H, Q365P, E375G, |
| | | D377E, F381Q, T382V, G384E, T385K, R405K, D409K, K410R, K411P, I412L, |
| | | S415G |
| 113 | 9 | F339S, A347D, E350P, Q353G, M354I, V355A, L357V, F362H, K364R, Q365P, |
| | | E375G, D377E, F381Q, T382V, G384E, T385K, R405K, D409K, K410R, K411P, |
| | | I412L, Y413K, S415G |
| 114 | 9 | F339S, D340E, A347D, E350P, Q353G, M354I, V355A, L357V, F362H, K364R, |
| | | Q365P, E375G, D377E, F381Q, T382V, G384E, T385K, R405K, D409K, K410R, |
| | | K411P, I412L, Y413K, S415G |
| 115 | 9 | F339S, D340E, A347G, E350P, Q353G, M354I, V355A, L357V, F362H, K364R, |
| | | Q365P, E375G, D377E, F381Q, T382V, G384E, T385K, R405K, D409K, K410R, |
| | | K411P, I412L, Y413K, S415G |
| 116 | 10 | M15Y, I17R, L21T, P23S, A24L, I29G, R35Y, L36M, I40K, T41G, D42R, V47I, R48I, |
| | | L53K, E54T, A55T, Y59L, V60Y, C61R, V62I, H81N, D97W, Q180P, A204D, T220M, |
| | | I234T, L242A, D246G, A250S, N251E, S253N, A258D, V260I, E282N, L285Q, |
| | | A287Q, F297Y, F298N, Q312A, K315V, T316V, I322V, L325F, L334M, F338Y, |
| | | K343D, P348N, T352L, H360M, E362K, C363D, M365N, P366L, V372C, A383M, |
| | | C390M, L395A, N399H, V400M, L408M, I410V, L411H, Y418Q, M419I, K420R, |
| | | A423C, T424D, A426L, G429S, A440N, A450N, P455S, M466W, N474S, H477N, |
| | | A480D, T481K, L485N, G488N, E489H, Q492E, V494M, M533L, F535A, L537M, |
| | | E540D, S543D, R544E, K545R, S547A, A559S, I565M, S567A, V578N, Q583N, |
| | | E597A, T598K, V603F, H604K, S611A, P617S, L621M, R622Q, A625E, L631V, |
| | | C633F, T635Q, K637N, H639D, I641F, L648N, D649G, V651Y, E653S, I658V, |
| | | V659C, P665A, W670I, N674D, I676G, S685K, G686K, F687A, E689I, T692I, |
| | | E693Q, V698K, A699R, I703G, T705G, I707W, Y709L, L710T, A711V |
| 117 | 10 | M15F, I17K, L21T, I29G, T31S, R35Y, E37R, I40K, T41G, D42T, A46T, R48V, L53Y, |
| | | R76C, L222I, L242A, D246G, N251K, S253N, V260M, E282N, A287Q, F297Y, |
| | | F298N, A299V, Q312A, K315I, I322V, L325F, F357Y, H360Y, C363D, M365N, |
| | | V372C, A383M, V392T, L395Q, N399H, L408M, I410V, Y418E, K420R, A423C, |
| | | T424D, A426I, G429S, I430F, L431M, F433Y, F462H, M466R, H477N, A480D, |
| | | T481K, G488N, Q492K, A493D, F535A, S543D, R544E, A559S, Q576N, V578D, |
| | | E597S, S611A, C633F, K637N, L648N, D649H, V651Y, I658V, V659C, I676G, |
| | | G686K, E689I, N701H, Y709M, A711Y |
| 118 | 10 | V2M, R5K, S7T, V12L, D14K, M15Y, I17R, L21T, I22M, P23S, A24L, I29G, M30K, |
| | | R35Y, L36M, T38V, I40K, T41G, D42R, V47I, R48I, Y51W, S52E, L53K, E54T, |
| | | A55D, Y59L, V60Y, C61K, V62I, H81N, D97Y, D98M, E99K, Q180P, A204D, T220M, |
| | | I234T, L242A, D246G, A250S, N251E, S253N, A258D, V260I, L271M, E282N, |
| | | K283H, L285Q, A287Q, N293D, F297Y, F298N, Q312A, K315V, T316V, I322V, |
| | | L325F, Q327E, L334M, F338Y, K343D, P348N, T352L, F357Y, H360M, D361N, |
| | | C363D, M365N, P366L, V372C, R381K, A383M, A388V, V392K, L395A, E396Q, |
| | | N399H, V400F, R404K, L408M, I410V, L411H, D414E, V416M, L417N, Y418E, |
| | | M419I, K420A, E421R, K422E, A423L, T424D, K425S, A426L, P428K, G429S, |
| | | A440G, A450N, A452Q, P455S, G461A, M466W, V471I, N474S, H477N, A480D, |
| | | T481K, L485N, G488N, E489H, S490K, Q492E, A493D, V494M, S523C, M533L, |
| | | F535A, L537M, E540D, S543D, R544E, K545R, S547A, I556V, A559S, I565M, |
| | | S567A, V578D, Q583N, E597A, T598E, F599Y, K600Y, V603F, H604K, S611A, |
| | | K614R, A615S, P617S, L621M, R622Q, A625E, L631V, C633F, T635Q, K637N, |
| | | H639G, L640V, I641L, R644W, L648N, D649N, V651Y, E653S, I658V, V659C, |
| | | H661C, P665A, D666N, W670I, N674D, I676G, S685K, G686K, F687A, E689I, |
| | | T692I, E693Q, T697R, V698K, A699Q, I703G, G704A, T705G, I707W, L708V, |
| | | Y709V, L710T, A711V |
| 119 | 10 | V2M, S7T, D14K, M15Y, I17K, L21T, P23S, I29G, M30K, T31S, R35Y, L36M, E37K, |
| | | I40K, T41G, D42H, V47I, R48V, L53E, E54T, A55D, V60F, C61K, V62L, R76C, |
| | | H81N, E99K, H211N, L222I, L242A, D246G, A250S, N251K, S253N, A258S, |
| | | V260M, G278S, E282N, K283N, A287Q, N293D, F297W, F298N, A299V, H309A, |
| | | Q312A, K315I, I322V, L325F, H328A, K343I, P348S, F357Y, F358Y, H360Y, C363D, |
| | | M365N, P366L, V372C, A383M, A388V, V391C, V392T, L395A, N399H, V400F, |
| | | L408M, I410V, V416M, L417N, Y418E, M419I, K422E, A423C, T424D, A426I, |
| | | G429S, I430F, L431M, F433Y, L439M, A440G, P444S, A450S, F462H, M466R, |
| | | H477N, A480D, T481K, G488N, E489G, S490K, A491E, Q492E, A493T, H496A, |
| | | I518V, E522S, M533L, S534A, F535A, E540D, S543E, R544E, K545R, I556V, |
| | | A559S, S567A, Q576N, V578D, D594H, R596K, F599Y, S611A, L631V, C633F, |
| | | T635Q, K637N, H639G, I641F, L648N, D649N, V651Y, E653S, I658V, V659C, |
| | | N674D, I676G, P677T, R679H, S685E, G686K, E689I, T692I, E693Q, K695V, |
| | | T697R, V698K, N701H, I703G, G704A, L708V |
| 120 | 10 | D14K, M15Y, I17K, I29G, T31S, R35Y, E37K, I40K, T41G, D42H, L45E, A46T, |
| | | R48V, A55E, R76C, H81N, L222I, L242A, D246G, A250S, N251K, S253N, A258S, |
| | | V260M, G278S, E282N, A287Q, F297Y, F298N, A299V, Q312A, K315I, I322V, |
| | | L325F, K343I, P348S, F357Y, F358Y, H360Y, C363D, M365N, P366L, V372C, |
| | | A383M, V391C, V392T, L395A, N399H, L408M, I410V, V416M, Y418E, M419I, |
| | | K420R, A423C, T424D, A426I, G429S, I430F, L431M, F433Y, L439M, A440N, |
| | | F462H, M466R, H477N, A480D, T481K, G488N, A491E, Q492K, A493D, F535A, |
| | | R544E, K545R, A559S, I565V, S567A, Q576N, V578D, D594H, R596K, E597S, |
| | | S611A, C633F, T635E, K637N, H639D, I641F, L648N, D649H, V651Y, I658V, |
| | | V659C, I676G, G686K, E689I, T692I, E693Q, V698K, N701H, I703G, Y709M, |
| | | L710M, A711Y |
| 121 | 10 | M15Y, I17K, L21T, P23S, A24L, I29G, T31S, R35Y, I40K, T41G, R48I, L53Y, V60F, |
| | | R76C, L222I, L242A, D246G, N251K, S253N, V260M, G278S, E282N, A287Q, |
| | | F297Y, F298N, A299V, Q312A, K315I, I322V, L325F, F338Y, K343I, F357Y, H360Y, |
| | | C363D, M365N, V372C, A383M, V391C, V392T, L395Q, N399H, L408M, I410V, |
| | | Y418E, K420R, A423C, T424D, A426I, G429S, I430F, L431M, F433Y, F462H, |
| | | M466R, H477N, A480D, T481K, G488N, Q492K, A493D, F535A, S543D, R544E, |
| | | A559S, Q576N, V578D, S611A, C633F, K637N, L648N, D649H, V651Y, I658V, |
| | | V659C, I676G, G686K, E689I, N701H, Y709M, A711Y |
| 122 | 10 | R5K, M15Y, I17K, L21T, I22M, P23S, A24L, I29G, R35Y, I40K, T41G, D42R, L45K, |
| | | R48V, L53H, E54T, A55E, Y59L, V60Y, V62I, D97W, Q180P, T220M, I234T, L242A, |
| | | D246G, A250S, N251E, S253N, A258D, V260I, E282N, L285Q, A287Q, F297Y, |
| | | F298N, A299V, Q312A, K315V, T316V, I322V, L325F, L334M, F338Y, K343S, |
| | | P348N, T352L, E362K, C363D, M365N, P366L, V372C, A383M, A388V, V392L, |
| | | L395A, N399H, L408M, I410V, V416M, Y418Q, M419I, K420A, A423C, T424D, |
| | | K425S, A426L, G429S, A450N, P455S, M466W, N474S, H477N, A480D, T481K, |
| | | L485N, A491F, V494M, F535A, S543E, R544D, I565M, V578N, Q583N, E597A, |
| | | T598K, V603F, S611A, P617S, R622Q, A625E, L631V, C633F, K637N, H639D, |
| | | I641F, L648N, D649N, V651Y, E653S, V659C, P665A, D666H, I676G, S685K, |
| | | G686K, F687A, E689I, T692I, E693Q, V698K, A699R, I703G, Y709V, L710M, A711V |
| 123 | 10 | V2M, S7T, D14K, M15Y, I17K, L21T, I29G, M30K, T31S, R35Y, L36M, E37K, I40K, |
| | | T41G, D42N, A46T, V47I, R48V, L53H, E54T, A55D, C61K, V62L, R76C, H81N, |
| | | E99K, H211N, L222I, L242A, D246G, A250S, N251K, S253N, S255T, A258S, |
| | | V260M, G278S, E282N, K283N, A287Q, N293D, F297W, F298N, A299V, H309A, |
| | | Q312A, K315I, I322V, L325F, H328A, K343I, P348S, F357Y, F358Y, H360Y, C363D, |
| | | M365N, P366L, V372C, A383M, A388V, V392T, L395A, N399H, V400F, L408M, |
| | | I410V, V416M, L417N, Y418E, M419I, K422E, A423C, T424D, A426I, G429S, I430F, |
| | | L431M, F433Y, L439M, A440G, P444S, A450S, F462H, M466R, H477N, A480D, |
| | | T481K, G488N, E489G, S490K, A491E, Q492E, A493T, H496A, G497A, I518V, |
| | | E522S, M533L, S534A, F535A, E540D, S543E, R544E, K545R, I556V, A559S, |
| | | S567A, Q576N, V578D, D594H, R596K, F599Y, S611A, L631V, C633F, T635Q, |
| | | K637N, H639D, I641F, L648N, D649N, V651Y, E653S, I658V, V659C, N674D, |
| | | I676G, P677T, R679H, S685E, G686K, E689I, T692I, E693Q, K695V, T697R, |
| | | V698K, N701H, I703G, G704A, L708V |
| 125 | 10 | D14K, I17K, L21T, P23S, A24L, I29G, T31S, R35Y, E37K, I40K, T41G, D42H, L45E, |
| | | R48I, L53Y, A55D, V60F, C61R, R76C, H81N, L222I, L242A, D246G, A250S, |
| | | N251K, S253N, S255T, A258S, V260M, G278S, E282N, A287Q, F297Y, F298N, |
| | | A299V, Q312A, K315I, I322V, L325F, K343I, P348S, F357Y, H360Y, C363D, |
| | | M365N, P366L, V372C, A383M, V391C, V392T, L395Q, N399H, L408M, I410V, |
| | | Y418E, M419I, K420R, A423C, T424D, A426I, G429S, I430F, L431M, F433Y, |
| | | L439M, A440N, A450C, F462H, M466R, H477N, A480D, T481K, L485T, G488N, |
| | | A491E, Q492K, A493D, F535A, S543E, R544E, K545R, A559S, S567A, Q576N, |
| | | V578D, D594H, R596K, E597S, S611A, C633F, T635E, K637N, H639D, I641F, |
| | | L648N, D649N, V651Y, I658V, V659C, I676G, G686K, E689I, T692I, E693Q, V698K, |
| | | N701H, I703G, L710M, A711I |
| 126 | 10 | D14K, M15Y, I17K, L21T, P23S, I29G, T31S, R35Y, E37K, I40K, T41G, D42H, |
| | | L45E, R48I, L53N, A55D, V60F, C61R, R76C, H81N, L222I, L242A, D246G, A250S, |
| | | N251K, S253N, A258S, V260M, E282N, K283G, A287Q, F297Y, F298N, A299V, |
| | | H309A, Q312A, K315I, I322V, L325F, P348S, F357Y, F358Y, H360Y, C363D, |
| | | M365N, P366L, V372C, A383M, V391M, V392T, L395Q, N399H, V400F, L408M, |
| | | I410V, V416M, L417N, Y418Q, M419I, K420R, A423C, T424S, A426I, G429S, |
| | | I430F, L431M, F433Y, L439M, A440G, A450C, M466R, H477N, A480D, T481K, |
| | | L485T, G488E, E489G, A491E, Q492E, A493T, H496A, I518V, E522S, M533L, |
| | | F535A, E540D, S543E, R544E, K545R, A559S, I565M, S567A, Q576N, V578D, |
| | | D594H, R596K, S611A, C633F, T635Q, K637N, H639D, I641F, L648N, D649N, |
| | | V651Y, E653S, I658V, V659C, I676G, P677T, R679H, G686K, T692I, E693Q, |
| | | T697R, V698K, N701H, I703G, L708V, L710M, A711Y |
| 127 | 10 | R5K, M15Y, I17K, I29G, I40K, T41G, D42H, R48V, L53H, D97H, Q180P, T220M, |
| | | I234T, L242A, D246G, A250N, N251E, S253N, A258D, V260I, G278S, E282N, |
| | | L285Q, A287Q, H296Q, F297Y, F298N, Q312A, K315V, T316V, I322V, L325F, |
| | | L334M, F338Y, P348S, T352L, C363D, M365N, P366L, V372C, A383M, A388V, |
| | | C390M, N399H, L408M, I410V, L411H, V416M, Y418E, M419I, K420A, A423C, |
| | | T424D, K425S, A426L, A450N, P455S, M466W, N474S, H477N, A480D, T481K, |
| | | A491F, V494M, S523C, F535A, S543E, R544E, I565M, V578D, Q583N, T598E, |
| | | V603F, S611A, K614R, R622Q, A625E, L631V, C633F, K637N, I641F, L648N, |
| | | D649N, V651Y, I658M, V659C, P665A, D666H, I676G, S685K, G686K, E689T, |
| | | A699R, I703G, Y709V, L710T, A711V |
| 128 | 10 | V2M, R5K, S7T, D14K, M15Y, I17R, L21T, I22M, P23S, A24L, I29G, M30K, R35Y, |
| | | L36M, T38V, I40K, T41G, D42R, V47I, R48I, Y51W, S52E, L53K, E54T, A55D, |
| | | Y59L, V60Y, C61K, V62I, H81N, D97Y, D98M, E99K, Q180P, A204D, T220M, I234T, |
| | | L242A, D246G, A250S, N251E, S253N, A258D, V260I, L271M, G278S, E282N, |
| | | K283G, L285Q, A287Q, N293D, H296Q, F297Y, F298N, Q312A, K315V, T316V, |
| | | I322V, L325F, Q327E, L334M, F338Y, K343D, P348H, T352L, F357Y, H360M, |
| | | D361N, C363D, M365N, P366Y, V372C, R381K, A383M, V392I, L395A, N399H, |
| | | V400M, L408M, I410V, L411H, D414E, V416M, L417N, Y418Q, M419I, K420R, |
| | | K422E, A423C, T424D, K425S, A426L, G429S, A440G, A450N, A452K, P455S, |
| | | G461A, M466W, V471I, N474S, H477N, A480D, T481K, L485N, G488N, E489H, |
| | | S490K, Q492E, A493D, V494M, S523C, M533L, F535A, L537M, E540D, S543E, |
| | | R544D, K545R, S547Q, I556V, A559S, I565M, S567A, V578D, Q583N, E597A, |
| | | T598K, F599Y, H604K, S611A, A615S, P617S, L621M, R622Q, A625E, L631V, |
| | | C633F, T635Q, K637N, H639D, I641F, R644W, L648N, D649G, V651Y, E653S, |
| | | I658V, V659C, P665A, D666N, W670I, N674D, I676E, S685K, G686K, F687A, |
| | | E689I, T692I, E693Q, T697R, V698K, A699Q, I703G, G704A, T705G, I707W, |
| | | L708V, Y709V, L710M, A711V |
| 129 | 10 | R5K, M15Y, L21T, P23S, A24L, I29G, R35Y, E37R, T41G, D42T, P44T, R48I, L53H, |
| | | V60Y, V62I, Q180P, T220M, L242A, A250N, N251E, S253N, A258D, V260I, G278S, |
| | | E282N, L285Q, A287Q, H296Q, F297Y, F298N, Q312A, K315V, T316V, I322V, |
| | | L325F, F338Y, P348H, T352L, E362K, C363D, M365H, V372C, A383M, C390M, |
| | | N399H, L408M, I410V, L411H, V416M, Y418Q, M419I, A423L, T424D, A426I, |
| | | P428R, A450N, P455S, H477N, A480D, A491F, V494M, S523C, F535A, S543E, |
| | | S547Q, I565M, V578D, Q583N, E597A, T598E, H604K, S611A, K614R, R622Q, |
| | | L631V, C633F, K637N, I641F, L648N, V651Y, V659C, P665A, D666H, I676G, |
| | | S685K, F687A, E689I, A699R, I703G, Y709V, L710M, A711V |
| 130 | 10 | R5K, M15Y, L21T, P23S, I29G, R35Y, E37R, T41G, D42T, P44T, R48I, L53H, V60Y, |
| | | V62I, D97W, Q180P, T220M, L242A, A250N, N251E, S253N, A258D, V260I, E282N, |
| | | L285Q, A287Q, F297Y, F298N, Q312A, K315V, T316V, I322V, L325F, L334M, |
| | | F338Y, P348H, T352L, F357Y, E362K, C363D, M365H, V372C, A383M, C390M, |
| | | N399H, L408M, I410V, L411H, V416M, Y418Q, M419I, A423L, T424D, A450N, |
| | | P455S, N474S, H477N, A480D, V494M, F535A, S543E, I565M, V578D, Q583N, |
| | | T598K, V603F, S611A, K614R, R622Q, L631V, C633F, K637N, L648N, V651Y, |
| | | V659C, H661C, I676G, S685K, F687A, E689I, A699R, I703G, Y709V, L710M, |
| | | A711V |
| 131 | 10 | M15Y, L21T, I29G, T31S, R35Y, E37R, I40K, T41G, R48I, R76C, L222I, L242A, |
| | | D246G, N251K, S253N, V260M, G278S, E282N, A287T, F298N, A299V, Q312A, |
| | | K315I, I322V, L325F, K343I, F357Y, H360Y, C363D, M365N, V372C, A383M, A388I, |
| | | V392C, L395Q, N399H, L408M, Y418E, K420R, A423C, T424D, A426I, G429S, |
| | | I430F, L431M, F433Y, F462H, M466R, H477N, L485T, A493D, F535A, S543D, |
| | | R544E, A559S, V578D, E597S, S611A, C633F, K637N, L648N, D649H, V651Y, |
| | | I658V, V659C, I676G, N701H, Y709M, A711Y |
| 132 | 10 | R5K, V12L, M15Y, I17K, L21T, I22M, P23S, I29G, R35H, L36M, I40K, T41G, D42R, |
| | | R48V, Y51W, L53H, E54T, A55D, Y59L, H81N, D97W, L146M, Q180P, T220M, |
| | | I234T, L242A, D246G, A250S, N251E, S253N, A258D, V260I, G278S, E282N, |
| | | L285Q, A287Q, H296Q, F297Y, F298N, Q312A, K315V, T316V, I322V, L325F, |
| | | L334M, F338Y, K343S, P348N, T352L, H360M, C363D, M365N, P366L, V372C, |
| | | A383M, L395A, N399H, V400M, L408M, I410V, L411H, Y418Q, M419I, K420R, |
| | | A423C, T424D, K425S, A426L, G429S, A440N, A450N, P455S, M466W, N474S, |
| | | H477N, A480D, T481K, L485N, G488N, E489H, Q492E, V494M, S523C, M533L, |
| | | F535A, L537M, S543D, R544D, K545R, A559S, I565M, S567A, V578N, Q583N, |
| | | E597A, T598K, V603F, H604K, S611A, R622Q, A625E, L631V, C633F, T635Q, |
| | | K637N, H639D, L640V, L648N, D649N, V651Y, E653S, I658V, V659C, H661C, |
| | | P665A, D666H, W670I, I676G, S685K, G686K, F687A, E689I, T692I, E693Q, |
| | | V698K, A699R, I703G, Y709V, L710T, A711V |
| 133 | 10 | R5K, M15Y, I17K, I29G, R35Y, T41G, D42H, R48I, L53H, D97W, Q180P, T220M, |
| | | L242A, A250N, N251E, S253N, A258D, V260I, E282N, L285Q, A287Q, F297Y, |
| | | F298N, Q312A, K315V, T316V, I322V, L325F, L334M, F338Y, P348H, T352L, |
| | | E362K, C363D, M365H, V372C, A383M, A388V, N399H, L408M, I410V, L411H, |
| | | V416M, Y418Q, M419I, A423L, T424D, A426I, P428R, A450N, P455S, N474S, |
| | | H477N, A480D, A491F, V494M, S523C, F535A, S543E, R544D, I565M, V578D, |
| | | Q583N, E597A, T598E, V603F, H604K, S611A, R622Q, A625E, L631V, C633F, |
| | | K637N, I641F, L648N, D649N, V651Y, I658M, V659C, P665A, D666H, I676G, |
| | | S685K, F687A, E689I, A699R, I703G, Y709V, L710T, A711V |
| 134 | 10 | M15Y, L21T, P23S, I29G, T31S, R35Y, T41G, R48I, L53Y, R76C, L222I, L242A, |
| | | N251K, S253N, V260M, G278S, E282N, A287T, F298N, A299V, Q312A, K315I, |
| | | I322V, L325F, K343I, F357Y, F358Y, H360Y, C363D, M365N, V372C, A383M, |
| | | A388I, V392T, L395Q, N399H, L408M, Y418E, K420R, A423C, T424D, A426I, |
| | | I430F, L431M, M466R, L485T, A493D, F535I, S543D, R544E, V578D, E597S, |
| | | S611A, K637N, L648N, D649H, V651Y, I658V, V659C, I676G, A711I |
| 135 | 10 | M15Y, L21T, P23S, I29G, T31S, R35Y, I40K, T41G, R48I, L53Y, R76C, L222I, |
| | | L242A, D246G, S253N, V260M, G278S, E282N, A287T, F298N, A299V, Q312A, |
| | | K315I, I322V, L325F, K343I, F357Y, H360Y, C363D, M365N, V372C, A383M, |
| | | V392T, L395Q, N399H, L408M, Y418E, K420R, A423C, T424D, A426I, G429S, |
| | | I430F, L431M, F433Y, F462H, M466R, H477N, L485T, A493D, F535A, R544E, |
| | | A559S, V578D, S611A, C633F, K637N, L648N, D649H, V651Y, I658V, V659C, |
| | | I676G, N701H, Y709M, A711Y |
| 136 | 10 | R5K, M15Y, I17K, I29G, I40K, T41G, D42H, L45K, R48V, L53H, D97W, Q180P, |
| | | T220M, I234T, L242A, D246G, N251E, S253N, A258D, V260I, E282N, L285Q, |
| | | A287Q, F297Y, F298N, Q312A, K315V, T316V, I322V, L325F, L334M, F338Y, |
| | | P348S, T352L, E362K, C363D, M365N, P366L, V372C, A383M, A388V, V392L, |
| | | N399H, L408M, I410V, V416M, Y418Q, M419I, K420A, A423C, T424D, K425S, |
| | | A426L, A450N, P455S, M466W, H477N, A480D, T481K, V494M, F535A, S543E, |
| | | R544D, I565M, V578N, Q583N, E597A, T598E, H604K, S611A, K614R, R622Q, |
| | | A625E, L631V, C633F, K637N, I641F, L648N, D649N, V651Y, I658M, V659C, |
| | | P665A, D666H, I676G, S685K, G686K, E689T, A699R, I703G, Y709V, L710T, |
| | | A711V |
| 137 | 10 | R5K, V12L, M15Y, I17K, L21T, I22M, P23S, I29G, R35Y, L36M, I40K, T41G, D42R, |
| | | R48I, Y51W, L53H, E54T, A55D, Y59L, V60Y, V62I, H81N, D97W, T220M, I234T, |
| | | L242A, D246G, A250S, N251E, S253N, A258D, V260I, E282N, L285Q, A287Q, |
| | | F297Y, F298N, A299V, Q312A, K315V, T316V, I322V, L325F, L334M, F338Y, |
| | | K343S, P348N, T352L, H360M, C363D, M365N, P366L, V372C, A383M, A388V, |
| | | V392L, L395A, N399H, V400M, L408M, I410V, L411H, V416M, Y418Q, M419I, |
| | | K420R, E421R, A423C, T424D, K425S, A426L, G429S, A440N, A450N, P455S, |
| | | G461H, M466W, N474S, H477N, A480D, T481K, L485N, G488N, E489H, Q492E, |
| | | V494M, S523C, M533L, F535A, L537M, S543D, R544E, K545R, L558T, A559S, |
| | | I565M, S567A, V578N, Q583N, E597A, T598E, V603F, H604K, S611A, P617S, |
| | | R622Q, A625E, L631V, C633F, T635Q, K637N, H639D, I641F, L648N, D649N, |
| | | V651Y, E653S, I658V, V659C, P665A, D666H, W670I, I676G, S685K, G686K, |
| | | F687A, E689I, T692I, E693Q, V698K, A699R, I703G, Y709V, L710M, A711V |
| 138 | 10 | M15Y, I17K, L21T, P23S, A24L, I29G, T31S, R35Y, E37K, I40K, T41G, D42H, R48I, |
| | | L53Y, V60F, R76C, H81N, L222I, L242A, D246G, S253N, A258S, V260M, G278S, |
| | | E282N, A287Q, F297W, F298N, A299V, Q312A, K315I, I322V, L325F, K343I, |
| | | F357Y, H360Y, C363D, M365N, P366L, V372C, A383M, A388I, V392T, L395A, |
| | | N399H, L408M, I410V, V416M, Y418E, A423C, T424D, A426I, G429S, I430F, |
| | | L431M, F433Y, A440N, F462H, M466R, H477N, A480D, T481K, L485T, G488N, |
| | | Q492K, A493D, F535A, S543D, R544E, A559S, I565V, Q576N, V578D, D594H, |
| | | R596K, S611A, C633F, T635E, K637N, H639D, L648N, D649H, V651Y, I658V, |
| | | V659C, I676G, G686K, E689I, T692I, V698K, L710M, A711Y |
| 139 | 10 | M15Y, I17K, L21T, P23S, A24L, I29G, R35Y, I40K, T41G, D42R, L45K, R48V, L53H, |
| | | E54T, A55E, Y59L, V60Y, V62I, D97H, Q180P, T220M, I234T, L242A, D246G, |
| | | A250S, N251E, S253N, A258D, V260I, E282N, L285Q, A287Q, F297Y, F298N, |
| | | Q312A, K315V, T316V, I322V, L325F, L334M, F338Y, K343S, P348N, T352L, |
| | | C363D, M365N, P366L, V372C, A383M, A388I, V392L, L395A, N399H, L408M, |
| | | I410V, L411H, V416M, Y418Q, M419I, K420A, A423C, T424D, K425S, A426L, |
| | | G429S, I430L, A450N, P455S, M466W, N474S, H477N, A480D, T481K, L485N, |
| | | A491F, V494M, F535A, S543E, R544D, S547Q, I565M, V578N, Q583N, E597A, |
| | | T598E, H604K, S611A, R622Q, A625E, L631V, C633F, K637N, H639D, I641F, |
| | | L648N, D649N, V651Y, E653S, V659C, P665A, D666H, I676G, S685K, G686K, |
| | | F687A, E689I, T692I, E693Q, V698K, A699R, V700L, I703G, Y709V, L710T, A711V |
| 140 | 10 | R5K, M15Y, I17K, L21T, P23S, A24L, I29G, R35Y, E37R, T41G, D42H, P44T, R48I, |
| | | L53H, V60Y, V62I, D97W, L146M, Q180P, T220M, L242A, A250N, N251E, S253N, |
| | | A258D, V260I, E282N, L285Q, A287Q, F297Y, F298N, Q312A, K315V, T316V, |
| | | I322V, L325V, L334M, F338Y, P348H, T352L, C363D, M365N, V372C, A383M, |
| | | C390M, N399H, L408M, I410V, L411H, V416M, Y418Q, M419I, A423L, T424D, |
| | | A426I, P428R, A450N, P455S, N474S, H477N, A480D, A491F, V494M, F535A, |
| | | S543E, R544D, I565M, V578D, Q583N, E597A, T598E, V603F, H604K, S611A, |
| | | K614R, R622Q, A625E, L631V, C633F, K637N, I641F, L648N, D649N, V651Y, |
| | | V659C, P665A, D666H, I676G, S685K, F687A, E689I, A699R, I703G, Y709V, |
| | | L710M, A711V |
| 141 | 10 | R5K, M15Y, I17R, L21T, P23S, I29G, R35Y, L36M, I40K, T41G, D42R, V47I, R48I, |
| | | Y51W, L53K, E54T, A55T, Y59L, V60Y, C61R, V62I, H81N, D97H, Q180P, A204D, |
| | | T220M, I234T, L242A, D246G, A250S, N251E, S253N, A258D, V260I, G278S, |
| | | E282N, L285Q, A287Q, H296Q, F297Y, F298N, A299V, Q312A, K315V, T316V, |
| | | I322V, L325F, L334M, F338Y, K343S, P348N, T352L, H360M, C363D, M365N, |
| | | P366L, V372C, A383M, A388V, L395A, N399H, V400M, L408M, I410V, L411H, |
| | | Y418Q, M419I, K420A, A423C, T424D, K425S, A426L, G429S, A440N, A450N, |
| | | P455S, M466W, N474S, H477N, A480D, T481K, L485N, G488E, E489H, Q492E, |
| | | V494M, M533L, F535A, L537M, E540D, S543E, R544D, K545R, S547A, A559S, |
| | | I565M, S567A, V578D, Q583N, E597A, T598E, H604K, S611A, A615S, L621M, |
| | | R622Q, A625E, L631V, C633F, T635Q, K637E, H639D, I641F, A642G, L648N, |
| | | D649G, V651Y, E653S, I658M, V659C, W670I, N674D, I676G, S685K, G686K, |
| | | F687A, E689I, T692I, E693Q, V698K, A699R, I703G, T705G, I707W, Y709V, |
| | | L710M, A711V |
| 142 | 10 | M15Y, L21T, I29G, T31S, R35Y, T41G, R48I, R76C, L222I, L242A, S253N, V260M, |
| | | G278S, E282N, A287T, F298N, A299V, Q312A, K315I, I322V, L325F, K343I, F357Y, |
| | | H360Y, C363D, M365N, V372C, A383M, A388I, V392T, L395Q, N399H, L408M, |
| | | Y418E, K420R, A423C, T424D, A426I, I430F, L431M, F462H, M466R, G488N, |
| | | Q492K, A493D, F535I, S543D, R544E, V578D, S611A, K637N, L648N, D649H, |
| | | V651Y, I658V, V659C, I676G, E689I, N701H, Y709M, A711I |
| 143 | 10 | D14K, M15Y, I17K, I29G, T31S, R35Y, E37K, I40K, T41G, D42H, L45E, A46T, |
| | | R48V, L53N, A55E, R76C, H81N, L222I, L242A, D246G, A250S, N251K, S253N, |
| | | A258S, V260M, G278S, E282N, K283G, A287Q, F297Y, F298N, A299V, H309A, |
| | | Q312A, K315I, I322V, L325F, K343I, P348S, F357Y, H360Y, C363D, M365N, |
| | | P366L, V372C, A383M, A388V, V392T, L395Q, N399H, V400F, L408M, I410V, |
| | | V416M, L417N, Y418Q, M419I, K420R, A423C, T424D, A426I, G429S, I430F, |
| | | L431M, F433Y, L439M, A440G, A450C, M466R, H477N, A480D, T481K, G488N, |
| | | E489G, A491E, Q492E, A493T, H496A, I518V, E522S, M533L, F535A, E540D, |
| | | S543D, R544E, K545R, A559S, S567A, Q576N, V578D, D594H, R596K, S611A, |
| | | C633F, T635Q, K637D, H639D, I641F, L648N, D649N, V651Y, E653S, I658V, |
| | | V659C, I676G, P677T, R679H, G686K, E689I, T692I, E693Q, T697R, V698K, |
| | | N701H, I703G, L708V, L710M, A711Y |
| 144 | 10 | 117K, I29G, T31S, R35Y, E37K, I40K, T41G, D42H, A46T, R48V, L53Y, R76C, |
| | | H81N, L222I, L242A, D246G, S253N, A258S, V260M, G278S, E282N, A287Q, |
| | | F297Y, F298N, A299V, Q312A, K315I, I322V, L325F, K343I, F357Y, F358Y, H360Y, |
| | | C363D, M365N, P366L, V372C, A383M, V391C, V392T, L395Q, N399H, L408M, |
| | | I410V, V416M, Y418E, K420R, A423C, T424S, A426I, G429S, I430F, L431M, |
| | | F433Y, A440N, F462H, M466R, H477N, A480D, T481K, G488N, Q492K, A493D, |
| | | F535A, R544E, A559S, Q576N, V578D, D594H, R596K, S611A, C633F, T635E, |
| | | K637N, H639D, L648N, D649H, V651Y, I658V, V659C, I676G, G686K, E689I, T692I, |
| | | V698K, L710M, A711Y |
| 145 | 11 | V66A, R71N, D105C, T122A, I142C, S225N, D230N, N232E, T236D, T246S, V247L, |
| | | Q250E, V251Q, S252A, M253E, R254K, P262A, K265R, V270T, N271D, S284A, |
| | | E286N, V302A, A313S, R328H, E338V, V341I, A344T, K347Q, V349I, S358N, |
| | | A362S, K372H, A374D, M380V, K389N, G424N, G427N, K435N, T444A, A462E, |
| | | E463D, L464W, Q474D, E475N, N477A, V490T, I493V, E523C, E532S, G535S, |
| | | D538C, V543I, K556S, T566A, L567A, Q570A, T571N, Y574F, F581Y, P594A, |
| | | T597G, T605Q, I607V, D613N, P632Y, T653V, S656G, A660N, S664C, I677M, |
| | | V683T, T685A, D687E, I690F, Y700H, Q705N, S708E, W710R, L771Q, Q773S, |
| | | P785A, E787D, T789S, L791T, D793Q, L805A, D813S, F814Y, L816F, I817T, |
| | | K819N, A822N, V823E, K824V, K825R, G826A, S827A, E828Q, I829L, A834Q, |
| | | G836A, V845A, M860A, I861L, S862A, A865T, E868D, A869T, L871M, T873D, |
| | | A879T, S881A, L882A, V885A, Q896H, V931K, R935G, I945L, A954V, E958Q, |
| | | L961M, K962Q, M964A, T965A, D985S, A987R, T988E, T1000A, T1004Q, M1015S, |
| | | Q1016R, S1020D, A1031V, T1047N, Q1069R, A1072E, E1087V, E1092G, A1096T |
| 146 | 11 | V66A, R71N, D105C, T122A, I142C, S225N, D230N, N232E, T236D, T246S, V247L, |
| | | Q250E, V251Q, S252A, R254K, P262A, K265R, V270T, N271D, S284A, E286N, |
| | | V302A, R328H, E338V, V341I, A344T, K347Q, V349I, S358N, A362S, D370E, |
| | | K372H, A374D, M380V, K389N, G424N, G427N, T429I, K435N, T444A, A462E, |
| | | E463D, L464W, Q474D, E475N, N477A, V490T, I493V, E523C, E532S, G535S, |
| | | D538C, V543I, Q552K, K556S, T566A, L567A, Q570A, T571N, Y574F, F581Y, |
| | | P594A, T597G, K601A, T605Q, I607V, D613N, P632Y, T653V, S656G, A660N, |
| | | S664C, I677M, V683T, T685A, D687E, I690F, Y700H, Q705N, S708E, W710R, |
| | | L771Q, Q773S, P785A, E787D, T789S, L791T, D793Q, L805A, D813S, F814Y, |
| | | L816F, I817T, K819N, A822N, V823E, K824V, K825R, G826S, S827A, E828Q, |
| | | I829L, A834Q, G836A, V845A, M860A, I861L, S862A, A865T, E868D, A869T, |
| | | L871M, T873D, A879T, S881A, L882A, V885A, Q896H, V931K, R935G, I945L, |
| | | E958Q, L961M, K962Q, M964A, T965A, D985S, A987R, T988E, T998A, T1000A, |
| | | T1004Q, M1015S, Q1016R, S1020D, A1031V, T1047N, Q1069R, A1072E, E1087V, |
| | | E1092G, A1096T |
| 147 | 11 | D105C, T122A, I142C, S225N, D230N, T236D, T246S, Q250E, V251Q, S252A, |
| | | M253E, R254K, P262A, V270T, N271D, S284A, V302A, E338V, V341I, A344T, |
| | | V349I, S358N, K372H, A374K, M380V, Q386Y, G424N, G427S, K435N, A462E, |
| | | L464W, Q474D, E475N, N477A, V490T, I493V, E523C, E532S, D538C, K556S, |
| | | T566A, L567Q, Q570A, Y574F, P594A, T597G, K601A, T605Q, D613N, P632Y, |
| | | T653V, S656G, A660N, S664C, T685A, I690F, Y700H, Q705N, S708E, W710R, |
| | | L771Q, Q773S, E787D, T789S, D793Q, L805A, L816F, I817T, K819S, A822N, |
| | | V823E, K824V, K825R, G826A, S827A, E828Q, I829L, A834Q, G836A, M860A, |
| | | S862A, E868D, A869T, T873D, L882A, Q896H, V931K, R935G, I945L, A954V, |
| | | E958Q, L961M, K962Q, M964A, D985S, T988E, T1000A, T1004Q, M1015S, |
| | | Q1016R, S1020D, A1031V, T1047N, E1087V, E1092G, A1096T |
| 148 | 11 | D105C, I142C, T236S, S284T, V341I, V349I, E523C, I524V, D538C, S664C, Q705H, |
| | | K825R, T1000A |
| 149 | 11 | D105C, I142C, S225N, T236D, T246S, S252A, P262A, N271D, S284A, V341I, |
| | | A344T, V349I, S358N, K435N, A462E, L464W, Q474D, N477A, V490T, E523C, |
| | | I524V, D538C, Q552K, K556S, L567A, Q570A, Y574F, P594A, T597G, P632Y, |
| | | S656G, S664C, T685A, I690F, Y700H, Q705N, L707N, S708E, W710R, L771Q, |
| | | Q773S, D793Q, L805A, L816F, I817T, K819S, A822N, V823E, K824V, K825R, |
| | | S827A, I829L, G836A, M860I, S862A, T873D, L882A, R935G, E958Q, L961M, |
| | | K962Q, D985S, T988E, T1000A, M1015S, S1020D, A1031V, E1087V, E1092G |
| 150 | 11 | D105C, I142C, S225N, D230N, T236D, T246S, V251Q, S252A, P262A, K265R, |
| | | V270K, N271D, S284A, Q336N, V341I, A344T, V349I, S358N, K435N, A462E, |
| | | L464W, Q474D, E475N, N477A, V490T, E523C, I524V, E532S, D538C, Q552K, |
| | | K556S, T566A, L567Q, Q570A, Y574F, D593N, P594A, T597G, P632Y, S656G, |
| | | A660N, S664C, T685A, I690F, Y700H, Q705N, L707N, S708E, W710R, L771Q, |
| | | Q773S, T789S, D793Q, L805A, L816F, I817T, K819S, A822N, V823E, K824V, |
| | | K825R, S827A, I829L, A834T, G836A, M860A, S862A, A869T, T873D, L882A, |
| | | R935G, E958Q, L961M, K962Q, M964A, D985S, T988E, T1000A, M1015S, S1020D, |
| | | A1031V, E1087V, E1092G |
| 151 | 11 | D105C, T122A, I142C, S225N, D230N, T236D, T246S, Q250E, V251Q, S252A, |
| | | M253E, R254K, P262A, K265R, V270T, N271D, S284A, V302A, Q336N, E338V, |
| | | V341I, A344T, V349I, S358N, K372H, A374K, M380V, G424N, G427S, K435N, |
| | | A462E, L464W, Q474D, E475N, N477A, V490T, I493V, E523C, E532S, D538C, |
| | | K556S, T566A, L567A, Q570A, Y574F, P594A, T597G, K601T, T605Q, D613N, |
| | | P632Y, T653V, S656G, A660N, S664C, T685A, I690F, Y700H, Q705N, L707N, |
| | | S708E, W710R, L771Q, Q773S, E787D, T789S, D793Q, L805A, L816F, I817T, |
| | | K819S, A822N, V823E, K824V, K825R, G826A, S827A, E828Q, I829L, A834Q, |
| | | G836A, M860A, S862A, E868D, A869T, T873D, L882A, Q896H, V931K, R935G, |
| | | I945L, A954V, E958Q, L961M, K962Q, M964A, D985S, T988E, T1000A, T1004Q, |
| | | M1015S, Q1016R, S1020D, A1031V, T1047N, E1087V, E1092G, A1096T |
| 152 | 11 | D105C, T122S, I142C, S225N, D230N, T236D, T246S, V251Q, S252A, P262A, |
| | | V270T, N271D, S284A, E338V, V341I, A344T, V349I, S358N, M380L, G427S, |
| | | K435N, A462E, L464W, Q474D, E475N, N477A, V490T, E523C, E532S, D538C, |
| | | Q552K, K556S, T566A, L567Q, Q570A, Y574F, P594A, T597G, T605A, P632Y, |
| | | S656G, A660N, S664C, T685A, I690F, Y700H, Q705N, S708E, W710R, L771Q, |
| | | Q773S, E787D, T789S, D793Q, L805A, L816F, I817T, K819S, A822N, V823E, |
| | | K824V, K825R, S827A, I829L, A834T, G836A, M860A, S862A, E868D, A869T, |
| | | T873D, L882A, V931K, R935G, A954V, E958Q, L961M, K962Q, M964A, D985S, |
| | | T988E, T1000A, M1015S, Q1016R, S1020D, A1031V, T1047N, E1087V, E1092G |
| 153 | 11 | D105C, I142C, S225N, T236S, S252A, P262A, S284A, V341I, V349I, K435N, |
| | | A462E, N477A, E523C, I524V, D538C, L567A, Q570G, Y574F, P594A, T597G, |
| | | P632Y, S656G, S664C, T685A, I690F, Q705N, L707N, S708E, Q773S, D793Q, |
| | | L805A, K819S, V823E, K824V, K825R, S827A, G836A, R935G, L961M, K962Q, |
| | | D985S, T988E, T1000A, M1015S, S1020D, A1031V |
| 154 | 11 | D105C, I142C, S225N, T236S, T246S, S252A, P262A, N271D, S284A, V341I, |
| | | V349I, K435N, A462E, L464W, Q474N, N477A, V490T, E523C, I524V, D538C, |
| | | Q552K, L567Q, Y574F, P594A, T597G, P632Y, S656G, S664C, T685A, I690F, |
| | | Y700H, Q705N, L707N, S708E, L771Q, Q773S, D793Q, L805A, K819S, V823E, |
| | | K824V, K825R, S827A, G836A, M860I, S862A, L882A, R935G, L961M, K962Q, |
| | | D985S, T988E, T1000A, M1015S, S1020D, A1031V, E1087V |
| 155 | 11 | D105C, I142C, S225N, D230N, T236D, T246S, V251Q, S252A, P262A, V270K, |
| | | N271D, S284A, V341I, A344T, V349I, S358N, K435N, A462E, L464W, Q474D, |
| | | E475N, N477A, V490T, E523C, I524V, E532S, D538C, K556S, T566A, L567Q, |
| | | Q570A, Y574F, P594A, T597G, K601A, P632Y, S656G, A660N, S664C, T685A, |
| | | I690F, Y700H, Q705N, S708E, W710R, L771Q, Q773S, T789S, D793Q, L805A, |
| | | L816F, I817T, K819S, A822N, V823E, K824V, K825R, S827A, I829L, A834T, |
| | | G836A, M860A, S862A, A869T, T873D, L882A, R935G, E958Q, L961M, K962Q, |
| | | M964A, D985S, T988E, T1000A, M1015S, S1020D, A1031V, E1087V, E1092G |
| 156 | 11 | D105C, T122S, I142C, S225N, D230N, T236D, T246S, V251Q, S252A, P262A, |
| | | V270T, N271D, S284A, Q336N, E338V, V341I, A344T, V349I, S358N, M380L, |
| | | G427S, K435N, A462E, L464W, Q474D, E475N, N477A, V490T, E523C, E532S, |
| | | D538C, K556S, T566A, L567A, Q570A, Y574F, P594A, T597G, T605A, P632Y, |
| | | S656G, A660N, S664C, T685A, I690F, Y700H, Q705N, L707N, S708E, W710R, |
| | | L771Q, Q773S, E787D, T789S, D793Q, L805A, L816F, I817T, K819S, A822N, |
| | | V823E, K824V, K825R, S827A, I829L, A834T, G836A, M860A, S862A, E868D, |
| | | A869T, T873D, L882A, V931K, R935G, E958Q, L961M, K962Q, M964A, D985S, |
| | | T988E, T1000A, M1015S, Q1016R, S1020D, A1031V, T1047N, E1087V, E1092G |
| 157 | 11 | D105C, I142C, S225N, T236S, S252A, P262A, S284A, V341I, V349I, K435N, |
| | | A462E, N477A, E523C, I524V, D538C, Q552K, L567A, Q570G, Y574F, P594A, |
| | | T597G, P632Y, S656G, S664C, T685A, I690F, Q705N, L707N, S708A, Q773S, |
| | | D793Q, L805A, K819S, V823E, K824V, K825R, S827A, G836A, R935G, L961M, |
| | | K962Q, D985S, T988E, T1000A, M1015S, S1020D, A1031V |
| 158 | 12 | P98A, S102R, K128A, Q153P, G236K, K260N, D261N, S283G, S325G, D379E, |
| | | S382A, A388T, V391I, V394I, T406K |
| 159 | 12 | D54A, P98A, S102R, K128A, Q153P, G176T, Q203E, A234G, G236K, K260N, |
| | | S283G, S325G, D379E, S382A, A388T, V390I, V391I, V394I, T406K |
| 160 | 12 | P98A, S102R, K128A, Q153P, G176T, Q203E, G236K, K260N, S283G, S325G, |
| | | D379E, S382A, A388T, V391I, V394I, T406K |
| 161 | 12 | D54A, P98A, S102R, K128A, Q153P, F163S, D167N, K174E, G176T, A199R, |
| | | Q203E, D227K, K231A, A234G, G236K, A245S, K260N, D261N, S283G, S291D, |
| | | S325G, D379A, S382A, A388T, V391I, V394I, D403A, K404E, T406K |
| 162 | 12 | D54A, P98A, S102R, K128A, Q153P, D167N, K174E, G176T, D227K, A234G, |
| | | K260N, D261N, S283G, S325G, D379A, S382A, A388T, V390I, V391I, V394I, T406K |
| 163 | 12 | D54A, P98A, S102Y, K128A, Q153P, A234G, N235K, G236K, K260N, S283G, |
| | | S325G, D379E, S382A, A388T, V390I, V391I, V394I, T406K |
| 164 | 12 | D54A, P98A, S102R, K128A, Q153P, Q203E, A234G, N235K, G236N, K260N, |
| | | S283G, S325G, D379E, S382A, A388T, V391I, V394I, T406K |
| 165 | 12 | P98A, S102R, K128A, Q153P, F163S, D167N, K174E, G176T, A199E, Q203K, |
| | | D227K, K231A, G236K, A245S, K260N, S283G, S291D, S325G, D379A, S382A, |
| | | A388T, V391I, V394I, D403A, T406K |
| 166 | 12 | P98A, S102R, K128A, Q153P, F163S, D167N, K174E, G176T, A199R, Q203E, |
| | | D227K, K231A, G236K, A245S, K260N, S283G, S291D, S325G, D379A, S382A, |
| | | A388T, V390M, V391I, V394I, D403A, T406K |
| 167 | 12 | D379E, V391I, V394I, T406K |
| 168 | 12 | P98A, S102Y, K128A, Q153P, G236K, K260N, D261N, S283G, S325G, D379E, |
| | | S382A, A388T, V391I, V394I, T406K |
| 169 | 12 | D54A, P98A, S102R, K128A, Q153P, D167N, K174E, G176T, Q203E, D227K, |
| | | N235K, G236K, K260N, S283G, S325G, D379A, S382A, A388T, V391I, V394I, |
| | | T406K |
| 170 | 12 | P98A, S102Y, K128A, Q153P, A234G, G236K, K260N, S283G, S325G, D379E, |
| | | S382A, A388T, V390I, V391I, V394I, T406K |
| 171 | 12 | D54A, P98A, S102Y, K128A, Q153P, F163S, D167N, K174E, G176T, A199E, |
| | | Q203K, D227K, K231A, G236K, A245S, K260N, S283G, S291D, S325G, G327N, |
| | | D379A, S382A, A388T, V391I, V394I, D403A, T406K |
| 172 | 12 | D54A, P98A, S102Y, K128A, Q153P, A234G, G236K, K260N, S283G, S325G, |
| | | D379E, S382A, A388T, V391I, V394I, T406K |
| 173 | 13 | I12L, T19Y, E24K, F33I, A72V, T75G, A101T, D102R, F152W, Q162D, K173A, |
| | | Q197A, N210M, V233Q, A247G, T274A, K276H, I280Q, G293V, T306L, D309E, |
| | | I312L, A355T, A374P, G392A, D393K, A395R, D398E, N400A, K412R |
| 174 | 13 | V6H, I12L, T19Y, E24K, D29S, F33I, A72V, T75G, A101T, D102E, A138W, E142Q, |
| | | M144V, F152W, Q162D, K173A, Q197A, N210M, V233Q, A247G, T263H, T274A, |
| | | K276H, H278M, I280Q, N284A, L291W, G293V, C299M, V303I, T306L, D309E, |
| | | I312L, D318E, Q353R, A355T, G392A, D393Q, A395R, T397G, D398E, N400A, |
| | | K412R, I424C |
| 175 | 13 | V6H, I12L, T19Y, E24K, D29S, F33I, A72V, T75G, A101T, D102K, F152W, Q162D, |
| | | K173A, Q197A, N210M, V233Q, A247G, T274A, K276H, I280Q, L291W, G293V, |
| | | V303I, T306L, D309E, I312F, D318E, Q353R, A355T, G392A, D393Q, A395K, |
| | | T397G, D398E, N400A, K412R, I424C |
| 176 | 13 | N2D, K3Q, V6H, E10Q, I12L, K17R, T19Y, E24K, D29A, V31Y, F33I, Q55H, I63V, |
| | | A72V, T75G, E98D, A101T, D102E, A138W, E142Q, L143H, M144I, F152W, |
| | | Q162D, K173A, Q177K, G178A, D184E, S191Y, Q197A, L201R, Q204G, S206H, |
| | | N210T, I224V, F230Y, V233Q, T237E, I243F, A247G, F249Y, T263H, E265Q, |
| | | V272I, A273N, T274A, K276H, H278M, I280Q, N284A, L286M, G293V, L297E, |
| | | D298Q, C299M, V303I, T306L, D309E, N311G, I312L, D318E, I320Q, F348W, |
| | | M349Q, Q353R, A355T, I372V, A374P, Q376M, S389M, G392A, D393E, A395R, |
| | | T397Q, D398E, N399H, N400A, H402M, K412R, F414Y, I424C |
| 177 | 13 | N2D, V6H, I12L, K17Q, T19Y, E24K, D29S, V31Y, F33I, A72V, T75G, A101T, |
| | | D102E, A124S, A138W, E142Q, L143Q, M144I, F152W, Q162D, K173A, D184K, |
| | | S191Y, Q197A, N210T, N221S, F230Y, V233Q, A236P, T237E, A247G, F249Y, |
| | | T263H, E265H, A273N, T274A, K276H, H278M, I280Q, N284A, L286M, L291W, |
| | | G293V, L297E, D298E, C299A, V303I, T306L, D309E, N311G, I312L, D318E, |
| | | I320Q, M349Q, Q353R, A355T, K371I, I372V, Q376M, S389M, G392A, D393Q, |
| | | A395R, T397S, D398E, N400A, K412R, F414Y, I424C |
| 178 | 13 | N2D, K3Q, V6H, E10Q, I12L, K17R, T19Y, E24K, D29S, V31Y, F33I, Q55H, I63V, |
| | | A72V, T75G, E98D, A101T, D102K, A138W, E142Q, L143H, M144I, F152W, |
| | | Q162D, K173A, Q177K, G178A, D184E, S191Y, Q197A, L201R, Q204G, S206H, |
| | | N210T, I224V, F230Y, V233Q, T237E, A247G, F249Y, T263H, E265H, V272I, |
| | | A273N, T274A, K276H, H278M, I280Q, N284A, L286M, L291W, G293V, L297E, |
| | | D298Q, C299A, V303I, T306L, D309E, N311G, I312L, I320Q, F348W, M349Q, |
| | | Q353R, A355T, K371I, I372V, A374P, Q376M, S389M, G392A, D393Q, A395R, |
| | | T397S, D398E, N399H, N400A, K412R, F414Y, I424C |
| 179 | 13 | V6H, I12L, T19Y, E24K, D29S, F33I, A72V, T75G, A101T, D102K, A138W, E142Q, |
| | | F152W, Q162D, K173A, Q197A, N210M, V233Q, A247G, T263H, T274A, K276H, |
| | | H278M, I280Q, N284A, L291W, G293V, C299M, V303I, T306L, D309E, I312L, |
| | | Q353R, A355T, G392A, D393R, A395R, T397G, D398E, N400A, K412R, I424C |
| 181 | 13 | V6H, I12L, T19Y, E24K, D29A, V31Y, F33I, A72V, T75G, A101T, D102E, A138W, |
| | | E142Q, M144I, F152W, Q162D, K173A, S191Y, Q197A, N210M, V233Q, T237E, |
| | | A247G, T263H, E265H, A273N, T274A, K276H, H278M, I280Q, N284A, L291W, |
| | | G293V, C299M, V303I, T306L, D309E, I312L, D318E, Q353R, A355T, G392A, |
| | | D393Q, A395R, T397G, D398E, N400A, K412R, I424C |
| 182 | 13 | I12L, T19Y, E24K, F33I, A72V, T75G, A101T, D102S, F152W, Q162D, K173A, |
| | | Q197A, N210M, V233Q, A247G, T274A, K276H, I280Q, G293V, T306L, D309E, |
| | | I312L, A355T, K371I, G392A, D393R, A395R, D398E, N400A, K412R |
| 183 | 13 | N2D, V6H, I12L, K17Q, T19Y, E24K, D29S, F33I, A72V, T75G, A101T, D102K, |
| | | A124S, A138W, E142Q, L143Q, M144R, F152W, Q162D, K173A, D184K, S191Y, |
| | | Q197A, N210T, N221S, F230Y, V233Q, T237E, A247G, F249Y, T263H, E265H, |
| | | A273N, T274A, K276H, H278M, I280Q, N284A, L286M, L291W, G293V, L297E, |
| | | D298E, C299A, V303I, T306L, D309E, N311G, I312L, D318E, I320Q, M349Q, |
| | | Q353R, A355T, I372V, Q376M, S389M, G392A, D393Q, A395R, T397S, D398E, |
| | | N400A, H402M, K412R, F414Y, I424C |
| 184 | 13 | V6H, I12L, T19Y, E24K, D29S, F33I, A72V, T75G, A101T, D102K, M144V, F152W, |
| | | Q162D, K173A, Q197A, N210M, V233Q, A247G, T274A, K276H, I280Q, L291W, |
| | | G293V, V303I, T306L, D309E, I312L, D318E, Q353R, A355T, K371I, A374P, |
| | | G392A, D393R, A395R, T397G, D398E, N400A, K412R, I424C |
| 185 | 13 | V6H, I12L, T19Y, E24K, D29S, V31Y, F33I, A72V, T75G, A101T, D102K, A138W, |
| | | E142Q, F152W, Q162D, K173A, S191Y, Q197A, N210M, V233Q, T237E, A247G, |
| | | T263H, E265H, A273N, T274A, K276H, H278M, I280Q, N284A, G293V, C299A, |
| | | V303I, T306L, D309E, I312F, Q353R, A355T, G392A, D393Q, A395R, T397G, |
| | | D398E, N400A, K412R, I424C |
| 186 | 13 | N2D, V6H, I12L, T19Y, E24K, D29S, V31Y, F33I, A72V, T75G, A101T, D102R, |
| | | A124S, A138W, E142Q, L143Q, F152W, Q162D, K173A, S191Y, Q197A, N210T, |
| | | N221S, F230Y, V233Q, T237E, A247G, T263H, E265H, A273N, T274A, K276H, |
| | | H278M, I280Q, N284A, L291W, G293V, C299M, V303I, T306L, D309E, I312L, |
| | | Q353R, A355T, G392E, D393Q, A395R, T397G, D398E, N400A, H402M, K412R, |
| | | F414Y, I424C |
| 187 | 13 | N2D, V6A, I12L, T19Y, R20D, E24K, D29S, F33I, A72V, T75G, A101T, D102K, |
| | | A124S, A138W, E142Q, L143Q, M144R, F152W, Q162D, K173A, S191Y, Q197A, |
| | | N210T, N221S, F230Y, V233Q, T237E, A247G, T263H, E265H, A273N, T274A, |
| | | K276H, H278M, I280Q, N284A, G293V, C299M, V303I, T306L, D309E, I312L, |
| | | D318E, Q353R, A355T, G392A, D393Q, A395R, T397G, D398E, N400A, H402M, |
| | | K412R, F414Y, I424C |
| 188 | 14 | L43F, T51H, T58Y, L69I, S88D, L95V, S245P, T256V, E276T, T306N, T321W |
| 189 | 14 | L43F, T51H, S56T, T58Y, L69I, S88D, S92A, L95V, Q143G, T211Q, G243N, S245P, |
| | | G253S, T256V, V261I, S271A, E276A, E279K, I288V, T306N, T321W |
| 191 | 14 | L43F, T51H, T58Y, L69I, S88D, L95V, Q143G, G243N, S245P, T256V, V261I, |
| | | S271A, E276A, I288V, T306N, T321W |
| 192 | 14 | L43F, T51H, T58Y, L69I, S88D, L95V, G243N, S245P, T256V, E276T, T306N, |
| | | T321W |
| 193 | 14 | L43F, T51H, S56T, T58Y, A64R, L69I, S88N, S90T, S92A, L95V, Q122K, Q143G, |
| | | S206N, L210V, T211Q, G243N, S245P, A250R, G253S, T256V, V261I, S271A, |
| | | E276A, E279K, I288V, T306N, N315Q, T321W |
| 194 | 14 | L43F, T51H, S56T, T58Y, A64R, L69I, S88N, S90T, S92A, L95V, Q122K, Q143G, |
| | | S204N, L210V, T211Q, G243N, S245P, A250R, G253A, T256V, V261I, S271A, |
| | | E276A, E279K, I288V, T306N, N315Q, T321W |
| 195 | 14 | L43F, T51H, T58Y, L69I, S88D, L95V, G243N, S245P, T256V, E276A, I288V, |
| | | T306N, T321W |
| 196 | 14 | L43F, T51H, T58Y, L69I, S88D, L95V, G243N, S245P, T256V, E276A, T306N, |
| | | T321W |
| 197 | 15 | D32H, T43S, A46W, N57T, T71S, F76N, A83K, W86Y, A87N, N91G, T95C, A102N, |
| | | T121S, N150K, S168G, K182N, L198M, K204Q |
| 198 | 15 | L198M, K204Q |
| 199 | 15 | D32N, T43N, A46W, N57T, T61N, F76N, A83K, V85T, W86Y, A87N, N91G, T95C, |
| | | A102N, T121S, N150K, S168G, K182Q, L198M, K204Q |
| 200 | 15 | D32H, T43S, A46Y, F76N, A87N, N91G, A102N, S168G, K182N, L198M, K204Q |
| 201 | 15 | D32H, T43S, A46W, F76N, A83K, A87N, N91G, A102N, S168G, K182N, L198M, |
| | | K204Q |
| 202 | 15 | D32H, T43N, A46W, N57T, F76N, A83K, A87N, N91G, A102N, S168G, K182N, |
| | | L198M, K204Q |
| 203 | 15 | D32H, T43S, A46W, N57T, F76N, A83K, A87N, N91G, A102N, S168G, K182N, |
| | | L198M, K204Q |
| 204 | 15 | D32H, T43N, A46Y, N57T, F76N, A83K, W86Y, A87N, N91G, T95C, A102N, S112N, |
| | | T121S, N150K, S168G, K182N, L198M, K204Q |
| 205 | 15 | D32N, T43S, A46W, N57T, T61N, F76N, A83K, V85T, W86Y, A87N, N91G, T95C, |
| | | A102N, T121S, N150K, S168G, A171T, K182N, L198M, K204Q |
| 206 | 15 | D32H, T43N, A46W, F76N, A83K, A87N, N91G, A102N, S168G, K182N, L198M, |
| | | K204Q |
| 207 | 15 | T43S, A46W, F76N, A83K, A87N, N91G, A102N, S168G, K182N, L198M, K204Q |
| 208 | 15 | D32H, T43N, A46W, N57T, F76N, A87N, N91G, A102N, S112N, T121S, S168G, |
| | | K182N, L198M, K204Q |
| 209 | 15 | D32H, T43N, A46Y, N57T, F76N, A83K, A87N, N91G, A102N, T121S, S168G, |
| | | K182Q, L198M, K204Q |
| 210 | 16 | N49S, G58K, T105Q, D136R, K147G, Q148R, D203S, T210S, V229T, A248Q, |
| | | S249T, E261S, T274A, V279A, D280N, L286C, V292C, A308S, D309S, E310S, |
| | | S311T, A317S, S318N, E323P |
| 211 | 16 | F39L, N49S, G58K, T105Q, D136R, K147G, Q148R, I162A, D203S, T210A, V229T, |
| | | A248Q, S249T, E261S, A273G, T274S, V279A, D280N, L286C, Q288V, V292C, |
| | | A308S, D309S, E310S, S311T, A317S, S318N, E323P |
| 212 | 16 | F39L, N49S, G58K, T105Q, L115T, D136R, K147G, Q148R, I162A, T210A, V229T, |
| | | A248Q, S249T, E261S, E267M, A273G, T274S, V279A, D280N, L286C, Q288V, |
| | | V292C, A308S, D309S, E310S, S311T, A317S, S318N, E323P |
| 213 | 16 | N49S, G58K, T105Q, D136R, K147G, Q148R, D203S, V229T, A248Q, E261S, |
| | | T274N, V279A, D280N, L286C, V292C, A308S, D309S, E310S, S311T, A317S, |
| | | S318N, E323P |
| 214 | 16 | K38L, F39L, N49S, G58K, N69D, F99Y, F102W, T105Q, G107N, I110V, L115T, |
| | | D136R, K147G, Q148R, L153C, I162A, F182Y, V183I, R184A, I201V, T210A, |
| | | V229M, A244G, A247D, A248Q, S249T, G250E, A251K, T260A, E261S, V265I, |
| | | A273G, T274S, V279A, D280N, L286C, Q288V, V292C, A308S, D309S, E310S, |
| | | S311T, A317S, S318N, E323P |
| 215 | 16 | N49T, G58K, T105Q, L115T, D136R, K147G, Q148R, V229T, A248Q, T274N, |
| | | D280N, L286C, V292C, A308S, D309S, E310S, S311T, A317S, S318N, E323P |
| 216 | 16 | N49S, G58K, T105Q, L115T, D136R, K147G, Q148R, T210S, V229T, A248Q, |
| | | S249T, E261S, T274N, V279A, D280N, L286C, V292C, A308S, D309S, E310S, |
| | | S311T, A317S, S318N, E323P |
| 217 | 16 | N49T, G58K, T105Q, D136R, K147S, Q148R, D203S, V229T, A248Q, T274N, |
| | | D280N, L286C, V292C, A308S, D309S, E310S, S311T, A317S, S318N, E323P |
| 218 | 16 | N49T, G58K, T105Q, L115T, D136R, K147G, Q148R, V229T, A248Q, L286C, |
| | | V292C, A308S, D309S, E310S, S311T, A317S, S318N, E323P |
| 219 | 16 | K38L, F39L, N49S, G58K, N69D, F99Y, F102W, T105Q, G107N, I110V, D136R, |
| | | K147G, Q148R, L153C, I162A, F182Y, V183I, R184A, I201V, D203S, T210A, |
| | | V229T, A244G, A247D, A248Q, S249T, G250E, A251K, T260A, E261S, V265I, |
| | | A273G, T274S, V279A, D280N, L286C, Q288V, V292C, A308S, D309S, E310S, |
| | | S311T, A317S, S318N, E323P |
| 220 | 16 | K38L, F39L, N49S, G58K, N69D, F102W, T105Q, G107N, D136R, K147G, Q148R, |
| | | I162A, F182Y, V183I, R184A, D203S, T210A, V229T, A248Q, S249T, E261S, |
| | | A273G, T274S, V279A, D280N, L286C, Q288V, V292C, A308S, D309S, E310S, |
| | | S311T, A317S, S318N, E323P |
| 221 | 16 | L286C, V292C, A308S, D309S, E310S, S311T, A317S, S318N, E323P |
| 222 | 17 | L2N, F7Y, E9R, A10P, S13N, S19D, T21N, Q22H, R24V, L27I, A29T, V35K, L40H, |
| | | A42G, E50Q, A55E, L56M, G58K, S62E, L72E, S76P, T87E, A93R, A103K, A157S, |
| | | R178K, P180D, E183K, E184D, A197K, H201N, L210F, A211K, P220E, D227E, |
| | | R253Q, Q257K, S276K, S279P, R280E, V288K, Q289H, A292H, F313Y, N328Y, |
| | | A333D, R340K, S344E, G361P, M364Q, S379A, T386K, E388K, H390T, R393Q, |
| | | A396H, T399N, A410N, G412A, A434E, K435R, R437K, L447M, T449V, L451C, |
| | | A461T, A463G, T464P, E475D, K477E, G482E, E489Q, R492E, H495K, R496K, |
| | | L497H, A498K, S499A, S507T, H509Y, Q513T, V523Y, Q524E, R535S, G536D, |
| | | K538E, L542F, S548T, E560K, Q568Y |
| 223 | 17 | F7Y, E9R, A10P, S13N, S19D, A29T, L40H, L56M, G58K, S62E, P180D, A197K, |
| | | H201Y, A211K, P220E, R253Q, S279P, A333D, G361N, M364Q, T386K, T399N, |
| | | A410N, G412A, L451C, A463G, G482E, R496E, L497H, A498K, S499A, V523Y, |
| | | G536D |
| 224 | 17 | F7Y, E9R, A29T, L40H, G58K, P180D, A197K, H201Y, P220E, R253Q, S279P, |
| | | G361N, M364Q, T386K, A396E, T399N, A410N, L451C, A463G, G482E, R496E, |
| | | A498K, G536D |
| 225 | 17 | F7Y, E9R, A10P, S13N, S19D, A29T, L40H, A42G, L56M, G58K, S62E, P180D, |
| | | A197K, H201Y, A211K, P220E, R253Q, S279D, N328Y, A333D, G361N, M364Q, |
| | | T386K, R393Q, T399N, A410N, G412A, A434D, T449V, L451C, A463G, G482E, |
| | | E489Q, R492E, R496K, L497H, A498K, S499A, L516I, V523Y, R535S, G536D |
| 226 | 17 | F7Y, E9R, A10P, S13N, S19D, A29T, L40H, L56M, G58K, S62E, P180D, A197K, |
| | | H201Y, A211K, P220E, R253Q, A333D, G361N, M364Q, T386K, A396E, T399N, |
| | | A410N, G412A, L451C, A463G, G482E, R496E, L497H, A498K, S499A, V523Y, |
| | | G536D |
| 227 | 17 | L2N, F7Y, E9R, A10P, S13N, S19D, T21K, L27I, A29T, V35K, L40H, A42G, A55E, |
| | | L56M, G58K, S62E, S76P, T87E, A93E, A103K, R178K, P180D, E183K, A197K, |
| | | H201N, L210F, A211Q, P220E, D227E, R253Q, Q257K, S276K, S279D, R280E, |
| | | V288K, A292H, F313Y, N328Y, A333D, G361R, M364Q, S379A, T386K, H390T, |
| | | R393Q, T399N, A410N, G412A, A434D, K435R, R437K, L447M, T449V, L451C, |
| | | A461T, A463G, E475D, K477E, G482E, E489Q, R492Q, H495K, R496E, L497H, |
| | | A498K, S499A, S507T, L516I, V523Y, Q524E, R535S, G536D |
| 228 | 17 | F7Y, E9R, A29T, L40H, L56M, G58K, S62E, P180D, A197K, H201Y, P220E, R253Q, |
| | | S279P, G361N, M364Q, T386K, T399N, A410N, L451C, A463G, G482E, R496E, |
| | | A498K, S499A, V523Y, G536D |
| 230 | 17 | L2N, F7Y, E9R, A10P, S13N, S19D, T21K, L27I, A29T, V35K, L40H, A42G, A55E, |
| | | L56M, G58K, S62E, S76P, T87E, A93E, A103K, R178K, P180D, E183K, A197K, |
| | | H201N, A211Q, P220E, D227E, R253Q, Q257K, S276K, S279P, R280E, V288K, |
| | | A292H, F313Y, N328Y, A333D, G361N, M364Q, S379A, T386K, H390T, R393Q, |
| | | T399N, A410N, G412A, A434D, K435R, R437K, L447M, T449V, L451C, A461T, |
| | | A463G, E475D, K477E, G482D, E489Q, R492E, H495K, R496K, L497H, A498K, |
| | | S499A, S507T, L516I, V523Y, Q524E, R535S, G536D |
| 231 | 17 | F7Y, E9R, A10P, S13N, S19D, A29T, L40H, A42G, A55E, L56M, G58K, S62E, |
| | | P180D, E183K, A197K, H201N, L210F, A211Q, P220E, R253Q, S276K, S279P, |
| | | A292H, N328Y, A333D, G361N, M364Q, T386K, H390N, R393Q, T399N, A410N, |
| | | G412A, A434D, T449V, L451C, A461E, A463G, G482E, E489Q, H495K, R496E, |
| | | L497H, A498K, S499A, L516I, V523Y, Q524E, R535S, G536D |
| 232 | 17 | F7Y, E9R, A10P, S13N, S19D, A29T, L40H, A42G, A55E, L56M, G58K, S62E, |
| | | P180D, E183K, A197K, H201N, A211K, P220E, R253Q, S276K, S279D, A292H, |
| | | N328Y, A333D, G361N, M364Q, T386K, H390N, R393Q, T399N, A410N, G412A, |
| | | A434D, T449V, L451C, A461E, A463G, G482E, E489Q, R492E, H495K, R496K, |
| | | L497H, A498K, S499A, L516I, V523Y, Q524E, R535S, G536D |
| 233 | 17 | F7Y, E9R, A29T, L40H, G58K, P180D, A197K, H201Y, P220E, R253Q, G361N, |
| | | M364Q, T386K, T399N, A410N, L451C, A463G, G482E, R496E, A498K, G536D |
| 234 | 17 | L2N, F7Y, E9R, A10P, S13N, S19D, T21K, L27I, A29T, V35K, L40H, A42G, A55E, |
| | | L56M, G58K, S62E, S76P, T87E, A93E, A103K, R178K, P180D, E183K, A197K, |
| | | H201N, A211Q, P220E, D227E, R253Q, Q257K, S276K, S279P, R280E, V288K, |
| | | A292H, F313Y, N328Y, A333D, G361N, M364Q, S379A, T386K, H390N, R393Q, |
| | | T399N, A410N, G412A, A434D, K435R, R437K, L447M, T449V, L451C, A461T, |
| | | A463G, E475D, K477E, G482E, E489Q, R492E, H495K, R496K, L497H, A498K, |
| | | S499A, S507T, L516I, V523Y, Q524E, R535S, G536D |
| 235 | 17 | L2N, F7Y, E9R, A10P, S13N, S19D, T21K, L27I, A29T, V35K, L40H, A42G, A55E, |
| | | L56M, G58K, S62E, S76P, T87E, A93E, A103K, R178K, P180D, E183K, A197K, |
| | | H201N, A211Q, P220E, D227E, R253Q, Q257K, S276K, S279P, R280E, V288K, |
| | | A292H, F313Y, A333D, G361N, M364Q, S379A, T386K, H390T, R393Q, T399N, |
| | | A410N, G412A, A434D, K435R, R437K, L447M, T449V, L451C, A461T, A463G, |
| | | E475D, K477E, G482D, E489Q, R492E, H495K, R496K, L497H, A498K, S499A, |
| | | S507T, L516I, V523Y, Q524E, R535S, G536D |
| 236 | 17 | L2N, F7Y, E9R, A10P, S13N, S19D, T21N, Q22H, R24V, L27I, A29T, V35K, L40H, |
| | | A42G, E50Q, A55E, L56M, G58K, S62E, L72E, S76P, T87E, A93R, A103K, A157S, |
| | | R178K, P180D, E183K, E184D, A197K, H201N, A211Q, P220E, D227E, R253Q, |
| | | Q257K, S276K, S279D, R280E, V288K, Q289H, A292H, F313Y, N328Y, A333D, |
| | | R340K, S344E, G361P, M364Q, S379A, T386K, E388K, H390T, R393Q, A396H, |
| | | T399N, A410N, G412A, A434E, K435R, R437K, L447M, T449V, L451C, A461T, |
| | | A463G, T464P, E475D, K477E, G482D, E489Q, R492Q, H495K, R496E, L497H, |
| | | A498K, S499A, S507T, H509Y, Q513T, L516I, V523Y, Q524E, R535S, G536D, |
| | | K538E, L542F, S548T, E560K, Q568Y |
| 237 | 17 | F7Y, E9R, A10P, S13N, S19D, A29T, L40H, A42G, L56M, G58K, S62E, P180D, |
| | | A197K, H201Y, A211E, P220E, R253Q, S279D, N328Y, A333D, G361R, M364Q, |
| | | T386K, R393Q, T399N, A410N, G412A, A434D, T449V, L451C, A463G, G482E, |
| | | E489Q, R492E, R496K, L497H, A498K, S499A, L516I, V523Y, R535S, G536D |
| 238 | 17 | F7Y, E9R, A29T, L40H, L56M, G58K, S62E, P180D, A197K, H201Y, P220E, R253Q, |
| | | G361N, M364Q, T386K, A396E, T399N, A410N, L451C, A463G, G482E, R496E, |
| | | A498K, S499A, V523Y, G536D |
| 239 | 18 | T42C, V48C, T72C, P83C, G89C, K90R, Y93C, S125N, K144Q, G162C, L169F, |
| | | D173E, N175S, A178K, D194C, P198C, S210K, N217C, L222W, T237C, D242C, |
| | | S243A, G247C, S253K, S283G, T292H, R296E, P316C, A325G, K329S, I341M, |
| | | R356N, S361C, A372Q, G376D, T393S |
| 240 | 18 | T42C, V48C, T72C, P83C, G89C, Y93C, E139N, G162C, N175S, N179S, N187A, |
| | | D194C, P198C, N217C, T237C, D242C, G247C, S283G, Q297R, P316C, A325G, |
| | | I341L, R356N, A357N, S361C, G376N, V380T, T393S |
| 241 | 18 | T42C, V48C, T72C, P83C, G89C, Y93C, E139N, G162C, D173E, N187A, D194C, |
| | | P198C, N217C, T237C, D242C, G247C, S283G, Q297R, P316C, R356N, S361C, |
| | | G376N, T393F |
| 242 | 18 | T42C, V48C, T72C, P83C, G89C, Y93C, E139N, G162C, N179S, N187A, D194C, |
| | | P198C, N217C, L222W, T237C, D242C, G247C, S283G, Q297R, P316C, A325G, |
| | | I341M, R356N, S361C, G376N, T393F |
| 243 | 18 | Y39S, T42C, V48C, T72C, P83C, E85G, G89C, K90R, Y93C, L113M, S125N, |
| | | K144Q, G162C, S166A, L167F, L169F, D173E, N175S, A178K, Y181L, D194C, |
| | | P198C, S210T, N217C, L222W, T237C, D242C, G247C, S253Q, S283G, T292H, |
| | | P316C, A325G, K329S, I341M, N345D, R356N, A357N, S361C, A372K, G376D, |
| | | T393S |
| 244 | 18 | T42C, V48C, T72C, P83C, G89C, K90R, Y93C, L113M, S125N, K144Q, G162C, |
| | | S166A, L167F, L169F, D173E, N175S, A178K, D194C, P198C, S210T, N217C, |
| | | L222W, T237C, D242C, S243A, G247C, S283G, T292H, P316C, A325G, K329S, |
| | | I341M, N345D, R356N, A357N, S361C, A372Q, G376D, T393S |
| 245 | 18 | T42C, V48C, T72C, P83C, G89C, Y93C, E139N, G162C, N187A, D194C, P198C, |
| | | N217C, T237C, D242C, G247C, S283G, Q297R, P316C, R356N, S361C, G376N, |
| | | T393F |
| 246 | 18 | T42C, V48C, T72C, P83C, E85G, G89C, Y93C, L113M, E139N, G162C, S166A, |
| | | L167F, L169F, N175S, N179S, Y181L, N187A, D194C, P198C, N217C, T237C, |
| | | D242C, G247C, S283G, T292H, Q297R, P316C, A325G, K329S, I341M, N345D, |
| | | R356N, A357N, S361C, A372K, G376N, V380T, Y381F, T393S |
| 247 | 18 | T42C, V48C, T72C, P83C, G89C, K90R, Y93C, S125N, K144Q, G162C, L169F, |
| | | D173E, N175S, A178K, D194C, P198C, S210K, N217C, L222W, T237C, D242C, |
| | | S243A, G247C, S283G, T292H, P316C, A325G, K329S, I341M, R356N, A357N, |
| | | S361C, N366H, A372Q, G376D, T393S |
| 248 | 18 | T42C, V48C, T72C, P83C, E85G, G89C, K90R, Y93C, L113M, S125N, K144Q, |
| | | G162C, S166A, L167F, L169F, D173E, N175S, A178K, Y181L, D194C, P198C, |
| | | S210T, N217C, L222W, A230S, T237C, D242C, G247C, S253Q, S283G, T292H, |
| | | K294N, P316C, A325G, K329S, I341M, N345D, R356N, A357N, S361C, A372K, |
| | | G376D, T393S |
| 249 | 18 | T42C, V48C, T72C, P83C, G89C, Y93C, E139N, G162C, S166A, D173E, N175S, |
| | | N179S, N187A, D194C, P198C, N217C, T237C, D242C, G247C, S283G, P316C, |
| | | A325G, K329S, I341L, N345D, R356N, A357N, S361C, G376N, V380T, T393S |
| 250 | 18 | T42C, V48C, T72C, P83C, G89C, Y93C, E139N, G162C, N179S, N187A, D194C, |
| | | P198C, N217C, T237C, D242C, G247C, S283G, Q297R, P316C, A325G, I341M, |
| | | R356N, S361C, G376N, T393F |
| 251 | 18 | T42C, V48C, T72C, P83C, G89C, Y93C, E139N, G162C, N187A, D194C, P198C, |
| | | N217C, T237C, D242C, G247C, S283G, P316C, R356N, S361C, G376N, T393F |
| 252 | 18 | T42C, V48C, T72C, P83C, G89C, K90R, Y93C, S125N, K144Q, G162C, L169F, |
| | | D173E, N175S, A178K, D194C, P198C, S210K, N217C, L222W, T237C, D242C, |
| | | G247C, S253K, S283G, T292H, R296E, P316C, A325G, K329S, I341M, R356N, |
| | | S361C, N366H, A372Q, G376D, T393S |
| 253 | 18 | T42C, V48C, T72C, P83C, G89C, Y93C, G162C, D194C, P198C, N217C, T237C, |
| | | D242C, G247C, P316C, S361C |
| 254 | 18 | T42C, V48C, T72C, P83C, G89C, K90R, Y93C, S125N, K144Q, G162C, L169F, |
| | | D173E, N175S, A178K, D194C, P198C, S210K, N217C, L222W, T237C, D242C, |
| | | S243A, G247C, S253K, S283G, T292H, K294N, P316C, A325G, K329S, I341M, |
| | | R356N, A357N, S361C, A372Q, G376D, T393S |
| 255 | 18 | T42C, V48C, T72C, P83C, G89C, Y93C, E139N, G162C, S166A, N175S, N179S, |
| | | N187A, D194C, P198C, N217C, T237C, D242C, G247C, S283G, Q297R, P316C, |
| | | A325G, K329S, I341L, N345D, R356N, A357N, S361C, G376N, V380T, T393S |
| 256 | 18 | T42C, V48C, T72C, P83C, G89C, Y93C, E139N, G162C, S166A, N179S, N187A, |
| | | D194C, P198C, N217C, T237C, D242C, G247C, S283G, Q297R, P316C, A325G, |
| | | I341M, R356N, A357N, S361C, G376N, T393S |
| 257 | 18 | T42C, V48C, T72C, P83C, G89C, Y93C, E139N, G162C, D173E, N187A, D194C, |
| | | P198C, N217C, T237C, D242C, G247C, S283G, P316C, R356N, S361C, G376N, |
| | | T393F |
| 258 | 18 | T42C, V48C, T72C, P83C, G89C, K90R, Y93C, S125N, K144Q, G162C, L169F, |
| | | D173E, A178K, D194C, P198C, N217C, L222W, T237C, D242C, G247C, S283G, |
| | | T292H, P316C, K329S, I341M, R356N, S361C, A372Q, G376D, T393S |
| 259 | 18 | T42C, V48C, T72C, P83C, G89C, K90R, Y93C, L113M, S125N, K144Q, G162C, |
| | | S166A, L167F, L169F, D173E, N175S, A178K, D194C, P198C, S210T, N217C, |
| | | L222W, T237C, D242C, G247C, S283G, T292H, P316C, A325G, K329S, I341M, |
| | | N345D, R356N, A357N, S361C, A372Q, G376D, T393S |
| 260 | 18 | T42C, V48C, T72C, P83C, G89C, K90R, Y93C, S125N, K144Q, G162C, L169F, |
| | | D173E, A178K, D194C, P198C, S210K, N217C, L222W, T237C, D242C, G247C, |
| | | S283G, T292H, K294N, P316C, K329S, I341M, R356N, S361C, A372Q, G376D, |
| | | T393S |
| 261 | 18 | T42C, V48C, T72C, P83C, G89C, Y93C, E139N, G162C, S166A, N179S, N187A, |
| | | D194C, P198C, N217C, T237C, D242C, G247C, S283G, Q297R, P316C, A325G, |
| | | R356N, A357N, S361C, G376N, T393S |
| 262 | 18 | T42C, V48C, T72C, P83C, G89C, Y93C, E139N, G162C, S166A, D173E, N175S, |
| | | N179S, N187A, D194C, P198C, N217C, T237C, D242C, G247C, S283G, Q297R, |
| | | P316C, A325G, R356N, A357N, S361C, G376N, V380T, T393S |
| 263 | 18 | T42C, V48C, T72C, P83C, E85G, G89C, Y93C, L113M, E139N, G162C, S166A, |
| | | L167F, L169F, D173E, N175S, N179S, Y181L, N187A, D194C, P198C, N217C, |
| | | T237C, D242C, G247C, S283G, T292H, P316C, A325G, K329S, I341M, N345D, |
| | | R356N, A357N, S361C, A372K, G376N, V380T, Y381F, T393S |
| 264 | 18 | T42C, V48C, T72C, P83C, G89C, K90R, Y93C, S125N, K144Q, G162C, L169F, |
| | | D173E, A178K, D194C, P198C, S210K, N217C, L222W, T237C, D242C, G247C, |
| | | S253K, S283G, T292H, R296E, P316C, K329S, I341M, R356N, S361C, N366H, |
| | | A372Q, G376D, T393S |
| 265 | 18 | T42C, V48C, T72C, P83C, G89C, K90R, Y93C, S125N, K144Q, G162C, L169F, |
| | | D173E, A178K, D194C, P198C, S210K, N217C, L222W, T237C, D242C, S243A, |
| | | G247C, S253K, S283G, T292H, P316C, K329S, I341M, R356N, S361C, N366H, |
| | | A372Q, G376D, T393S |
| 266 | 18 | T42C, V48C, T72C, P83C, G89C, K90R, Y93C, S125N, K144Q, G162C, S166A, |
| | | L169F, D173E, N175S, A178K, D194C, P198C, S210K, N217C, L222W, T237C, |
| | | D242C, G247C, S283G, T292H, P316C, A325G, K329S, I341M, N345D, R356N, |
| | | A357N, S361C, A372Q, G376D, T393S |
| 267 | 19 | I44V, T47N, K55N, P56K, L67H, Y73C, D77A, K80D, I88A, N110A, L122R, I124M, |
| | | I128V, N141H, S154A, L155K, S158N, S159T, A167C, D173G, S189D, D195G, |
| | | T202V, D216A, D217N, K220N, A222S, A227T, G236K, Q237S, F238W, D244S, |
| | | S248Q, I253V, Q273E, E276R, N279D, Y280W, P315R, T324K, V326Y, N329A, |
| | | R331L |
| 268 | 19 | I44V, K55N, P56K, Y73C, D77A, K80D, I88A, I103F, N110A, I124M, N134T, N141H, |
| | | S154N, L155K, S158N, S159T, D173G, S189A, D195G, T202V, D216A, D217N, |
| | | K220N, A222D, A227T, G236K, Q237S, F238W, D244S, S248Q, I253V, Q273E, |
| | | E276R, N279D, Y280W, P315R, T324K, R331L |
| 269 | 19 | K80M, I88A, I124M, D173G, S189D, D195G, T202V, A227T, D244S, S248K, |
| | | Y280W, P315R, T324K, R331L |
| 270 | 19 | I44V, P56A, Y73C, D77A, K80D, I88A, I103F, N110A, K120L, I124M, S154N, L155K, |
| | | S159T, D173G, S189D, D195G, T202V, D216A, D217N, K220N, A222D, A227T, |
| | | F238W, D244S, S248K, Q273E, Y280W, P315R, T324K, R331L |
| 271 | 19 | I44V, I62V, Y73C, V74Y, D77A, K80D, L82F, I88A, F91M, K113T, I124M, L155K, |
| | | S158N, S189D, D195G, T202C, D213S, D216A, D217N, K220N, A227T, F238W, |
| | | D244R, D262S, Q273E, Y280W, N290S, S300D, P315R, T324K, V326Y, N329Q |
| 272 | 19 | I44V, I62V, V74Y, D77A, K80D, L82F, I88C, F91M, K113T, I124M, L155K, S158N, |
| | | S189N, D195G, T202C, D216A, D217N, K220N, A227T, F238W, D244R, D262T, |
| | | Y280W, N290S, P315R, T324K, V326Y, N329Q |
| 273 | 19 | I44V, Y73C, K80D, I88A, I124M, L155K, D173G, D195G, T202V, D216A, D217N, |
| | | K220N, A222D, A227T, F238W, D244S, S248K, Q273E, Y280W, P315R, T324I, |
| | | R331L |
| 274 | 19 | I44V, P56A, Y73C, D77A, K80D, I88A, N110A, I124M, S154N, L155K, S159T, |
| | | D173G, S189D, D195G, T202V, D216A, D217N, K220N, A222D, A227T, F238W, |
| | | D244S, S248K, Q273E, Y280W, P315R, T324K, R331L |
| 275 | 19 | I44V, K80D, I88A, I103F, I124M, N134T, S154N, L155K, D173G, D195G, T202V, |
| | | K220N, A222D, A227T, D244S, S248Q, Y280W, P315R, T324K, R331L |
| 276 | 19 | I62V, V74Y, K80D, L82F, I88C, F91M, K113T, I124M, S189N, D195G, T202C, |
| | | K220N, A227T, D244R, D262T, Y280W, P315R, T324K, V326Y |
| 277 | 19 | P315R, R331L |
| 278 | 19 | I44V, I62V, V74Y, D77A, K80D, L82F, I88C, F91M, K113T, I124M, L155K, S158N, |
| | | D195G, T202C, D216A, D217N, K220N, A227T, F238W, D244R, D262T, Y280W, |
| | | N290S, P315R, T324K, V326Y |
| 279 | 19 | I44V, T47N, K55N, P56K, Y73C, D77A, K80D, I88A, I103F, N110A, L122R, I124M, |
| | | I128V, N134T, N141H, S154A, L155K, S158N, S159T, A167C, D173G, S189A, |
| | | D195G, T202V, D216A, D217N, K220N, A222D, A227T, G236K, Q237S, F238W, |
| | | D244S, S248Q, I253V, Q273E, E276R, N279D, Y280W, P315R, T324K, V326Y, |
| | | N329A, R331L |
| 280 | 19 | K80M, I88A, I103F, I124M, D173G, S189D, D195G, T202V, A227T, D244S, S248K, |
| | | Y280W, P315R, T324K, R331L |
| 281 | 19 | I44V, K55N, P56K, Y73C, D77A, K80D, I88A, I103F, N110A, I124M, N141H, L155K, |
| | | S158N, S159T, D173G, S189A, D195G, T202V, D216A, D217N, K220N, A222D, |
| | | A227T, G236K, Q237S, F238W, D244S, S248Q, I253V, Q273E, E276R, N279D, |
| | | Y280W, P315R, T324K, R331L |
| 282 | 19 | I44V, K45C, K55N, I62M, L67T, Y73C, V74Y, D77A, K80D, L82F, R83A, I88A, |
| | | F91M, I103M, N110A, K113T, I124M, K150T, L155K, S158N, S159T, S189N, |
| | | D195G, T202C, D213S, D216A, D217N, K220N, A222D, A227T, F238W, D244R, |
| | | S248N, D262T, Y270N, Q273E, Y280W, N290G, S300D, P315R, T324K, V326Y, |
| | | N329Q |
| 283 | 19 | I44V, K80D, I88A, I124M, L155K, D173G, D195G, T202V, K220N, A222D, A227T, |
| | | D244S, S248K, Y280W, P315R, T324K, R331L |
| 284 | 19 | I44V, Y73C, K80D, I88A, I103F, I124M, S154N, L155K, D173G, D195G, T202V, |
| | | D216K, K220N, A222D, A227T, F238W, D244S, S248K, Q273E, Y280W, P315R, |
| | | T324K, R331L |
| 285 | 19 | I44V, K45C, K55N, I62M, L67T, Y73C, V74Y, D77A, K80D, L82F, R83A, I88A, |
| | | F91M, I103M, N110A, K113T, I124M, K150T, L155K, S158N, S159T, S189D, |
| | | D195G, T202C, D213K, D216A, D217N, K220N, A222D, A227T, F238W, D244Q, |
| | | S248N, D262S, Y270N, Q273E, Y280W, N290G, P315R, T324K, V326Y, N329Q |
| 286 | 19 | I62V, V74Y, K80D, L82F, I88A, F91M, K113T, I124M, D195G, T202C, K220N, |
| | | A227T, D244R, D262T, Y280W, N290S, P315R, T324K, V326Y, N329Q |
| 287 | 19 | I44V, K45C, T47N, K55N, I62M, L67T, Y73C, V74Y, D77A, K80D, L82F, R83A, |
| | | D84H, G87N, I88A, F91M, I103M, N110A, K113T, K120T, L122R, I124M, N141H, |
| | | K150T, L155K, S158N, S159T, K177A, K181R, S189D, D195G, T202C, T206D, |
| | | N212D, D213E, D216A, D217N, K220N, A222S, A227T, Q237D, F238W, D244R, |
| | | S248N, I253V, G259A, D262S, Y270N, Q273E, E276R, Y280W, N290G, P315R, |
| | | T324K, V326Y, N329Q |
| 288 | 19 | I44V, I62V, Y73C, V74Y, D77A, K80D, L82F, I88A, F91M, K113T, I124M, L155K, |
| | | S158N, S189D, D195G, T202C, D213S, D216A, D217N, K220N, A227T, F238W, |
| | | D244R, D262T, Q273E, Y280W, N290S, S300D, P315R, T324K, V326Y, N329Q |
| 289 | 19 | I62V, V74Y, K80D, L82F, I88A, F91M, K113T, I124M, S189N, D195G, T202C, |
| | | K220N, A227T, D244R, D262T, Y280W, N290S, P315R, T324K, V326Y |
| 290 | 19 | I44V, I62V, Y73C, V74Y, D77A, K80D, L82F, I88A, F91M, K113T, I124M, L155K, |
| | | S158N, S189D, D195G, T202C, D213E, D216A, D217N, K220N, A222D, A227T, |
| | | F238W, D244Q, S248N, D262S, Y270N, Q273E, Y280W, N290S, P315R, T324K, |
| | | V326Y, N329Q |
| 291 | 19 | I44V, I62V, Y73C, V74Y, D77A, K80D, L82F, I88C, F91M, K113T, I124M, L155K, |
| | | S158N, K177Q, D195G, T202C, D213S, D216A, D217N, K220N, A222D, A227T, |
| | | F238W, D244R, S248N, G259A, D262S, Y270N, Q273E, Y280W, N290S, P315R, |
| | | T324K, V326Y, N329Q |
| 292 | 19 | I44V, K45C, T47N, K55N, I62M, L67T, Y73C, V74Y, D77A, K80D, L82F, R83A, |
| | | D84H, G87N, I88A, F91M, I103M, N110A, K113T, K120T, L122R, I124M, N141H, |
| | | K150T, L155K, S158N, S159T, K177A, K181R, S189D, D195G, T202C, T206D, |
| | | N212D, D213E, D216A, D217N, K220N, A222S, A227T, Q237D, F238W, D244R, |
| | | S248N, I253V, D262T, Y270N, Q273E, E276R, Y280W, N290G, S300D, P315R, |
| | | T324K, V326Y, N329Q |
| 293 | 20 | T39Y, Y49W, T85K, T119V, I120M, G121R, A136F, G146L, D153R, A171K, D190H, |
| | | E204Y, G231E, A301E, I312A, R316K, D348T, L383T |
| 294 | 20 | F3Y, R10K, T39Y, Y49W, G62D, T66A, S70K, R76K, A81D, T85K, A106S, T119V, |
| | | I120M, G121R, I123L, A136M, G146L, D153R, G166A, E167D, A171E, D190H, |
| | | L192Y, V196T, E204Y, Y212F, L226H, G231E, A238H, A301E, I312A, D315E, |
| | | R316K, A318I, A319K, Q337E, D348A, A355S, H374K, L383T |
| 295 | 20 | T39Y, Y49W, T85K, I120M, G121R, A136F, G146L, D153R, A171K, D190H, E204Y, |
| | | G231E, A301E, I312A, R316K, D348T, L383T |
| 296 | 20 | F3Y, T39Y, Y49W, A81D, T85K, T119V, I120M, G121R, A136F, G146L, D153R, |
| | | A171E, D190H, E204Y, Y212F, G231E, A301E, I312A, D315E, R316K, A319K, |
| | | D348A, A355S, L383T |
| 297 | 20 | T39I, Y49W, T85K, T119V, I120M, G121R, A136F, A171K, D190H, E204Y, G231E, |
| | | A301E, R316K, D348T, L383T |
| 298 | 20 | F3Y, R10K, T39Y, Y49W, G62D, Q77D, A81D, T85K, T119V, I120M, G121R, I123L, |
| | | A136F, A143F, D153R, A171E, D190H, Y212F, G231E, A301E, I312A, D315E, |
| | | R316K, A319K, D348A, A355S, H374W, L383T |
| 299 | 20 | F3Y, T39Y, Y49W, A81D, T85K, T119V, I120M, G121R, A136F, D153R, A171E, |
| | | D190H, Y212F, G231E, A301E, I312A, D315E, R316K, A319K, D348A, A355S, |
| | | L383T |
| 300 | 20 | T39Y, Y49W, T85K, T119V, I120M, G121R, A136M, G146L, A171K, D190H, E204Y, |
| | | G231E, A301E, R316K, D348T, L383T |
| 301 | 20 | F3Y, T39Y, Y49W, T85K, T119V, I120M, G121R, A136F, G146L, D153R, A171E, |
| | | D190H, E204Y, G231E, A301E, I312A, R316K, D348A, A355S, L383T |
| 303 | 20 | F3Y, R10K, T39Y, Y49W, G62D, T66A, S70K, R76K, A81D, T85K, A106S, I120M, |
| | | G121R, A136M, A143T, G146L, D153R, G166A, E167D, A171E, L176I, D190H, |
| | | L192Y, V196T, Y212F, L226H, G231E, A238H, A301E, I312A, R316K, A318R, |
| | | Q337E, D348A, A355S, H374K, L383T |
| 304 | 20 | F3Y, R10K, T39Y, Y49W, G62D, T66A, S70K, R76K, A81D, T85K, A89V, A106S, |
| | | T119V, I120M, G121R, I123L, A136F, D153R, G166A, A171E, L176I, D190H, |
| | | L192Y, V196A, Y212F, G231E, A238H, A301E, I312A, D315E, R316K, A318R, |
| | | D348A, A355S, H374K, L383T |
| 305 | 20 | F3Y, R10K, T39Y, G62D, T66A, S70K, R76K, A81D, T85K, A89V, A106S, T119V, |
| | | I120M, G121R, I123L, A136F, D153R, G166A, A171E, L176I, D190H, L192Y, |
| | | Y212F, G231E, A238H, A301E, I312A, D315E, R316K, A318R, D348A, A355S, |
| | | H374K, L383T |
| 306 | 20 | F3Y, R10K, T39Y, G62D, Q77D, A81D, T85K, A89V, T119V, I120M, G121R, I123L, |
| | | A136F, A143F, D153R, A171E, L176I, D190H, V196I, Y212F, A301E, I312A, D315E, |
| | | R316K, A319K, D348A, A355S, H374W, L383T |
| 307 | 21 | Q38A, S52G, I70M, A84D, A91P, T100S, R183E, D198H, N293Y, Q350R, H351R, |
| | | A400P, S460H, D489P, N502S |
| 308 | 21 | S25R, Q38A, S52G, I70M, A84D, A91P, T100S, S132Q, Q133G, R183E, N187R, |
| | | D198H, A206T, N293Y, V304E, V346I, Q350R, H351R, A400P, E453P, S460H, |
| | | D489P, N502S |
| 309 | 21 | S25R, D37K, Q38A, S52G, N69P, I70M, A84E, A91P, D92S, T100S, D115T, A117T, |
| | | S132Q, Q133G, V143T, R183E, N187K, D198N, A206D, S221Q, E246N, A286P, |
| | | N293Y, V304E, V346I, Q350R, H351R, T358S, A371Q, A395K, A400P, K450Q, |
| | | E453P, G455S, S460H, Q476D, D489E, N502S, S507C, S512Y |
| 311 | 21 | Q38A, S52G, I70M, A84D, A91P, T100S, R183E, D198H, A281P, N293Y, V346I, |
| | | Q350R, H351R, A400P, S460H, N502S |
| 312 | 21 | S25R, D37K, Q38A, S52G, W54Y, T63Y, I70M, M81L, A84D, A91P, D92S, T100S, |
| | | D115T, A117D, T129N, S132Q, Q133G, V143T, R183E, Q184E, N187K, D198N, |
| | | A206T, S221Q, E246N, A286P, N293Y, V304E, T329N, V346I, Q350R, H351R, |
| | | T358S, A371T, A395E, A400P, A412G, A425N, K450T, E453P, G455S, S460H, |
| | | Q476D, D489E, N502S, S507C, S512Y |
| 313 | 21 | S25R, Q38A, S52G, I70M, A84D, A91P, T100S, S132Q, Q133G, R183E, N187R, |
| | | D198H, A206T, A281P, N293Y, V304E, V346I, Q350R, H351R, A400P, E453P, |
| | | S460H, D489P, N502S |
| 314 | 21 | S25R, D37K, Q38A, S52G, T63Y, I70M, A84E, A91P, D92S, T100S, D115T, A117T, |
| | | S132Q, Q133G, V143T, R183E, N187K, D198N, A206T, S221Q, E246N, A286P, |
| | | N293Y, V304E, V346I, Q350R, H351R, T358S, A371Q, A395K, A400P, K450Q, |
| | | E453P, G455S, S460H, Q476D, D489E, N502G, S507C, S512Y |
| 315 | 21 | S25T, D37K, Q38A, S52G, W54Y, T63Y, I70M, A84E, A91P, D92S, T100S, D115T, |
| | | A117K, T129N, S132Q, Q133G, V143T, R183E, Q184E, N187K, D198N, A206T, |
| | | S221Q, E246N, A286P, N293Y, V304E, T329N, Q350R, H351R, T358S, S364G, |
| | | A371T, A395K, A400P, A412G, A425N, K450T, E453P, G455S, S460H, Q476D, |
| | | D489E, N502S, S507C, S512Y, V513I |
| 316 | 21 | S25R, D37K, Q38A, S52G, T63Y, I70M, A84D, A91P, D92S, T100S, D115T, A117T, |
| | | S132Q, Q133G, R183E, N187K, D198H, A206D, S221Q, E246N, A286P, N293Y, |
| | | V304E, V346I, Q350R, H351R, A371Q, A395K, A400P, E453P, S460H, D489E, |
| | | N502S, S507C, S512Y |
| 317 | 21 | S25R, D37K, Q38A, S52G, N69P, I70M, A84D, A91P, T100S, D115T, A117T, |
| | | S132Q, Q133G, R183E, N187Q, D198H, A206T, S221Q, A286P, N293Y, V304E, |
| | | V346I, Q350R, H351R, A400P, E453P, S460H, D489E, N502S, S512Y |
| 318 | 21 | S25R, D37K, Q38A, S52G, T63Y, I70M, A84D, A91P, D92S, T100S, D115T, A117T, |
| | | S132Q, Q133G, R183E, N187K, D198H, A206T, S221Q, E246N, A286P, N293Y, |
| | | V304E, V346I, Q350R, H351R, A371Q, A395K, A400P, E453P, S460H, D489E, |
| | | N502S, S507C, S512Y |
| 319 | 21 | S25R, D37K, Q38A, S52G, T53V, W54Y, F58Y, T63Y, I70M, A84E, T88V, A91P, |
| | | D92S, T100S, T102S, D115T, A117K, T129N, T130K, S132Q, Q133G, F141Y, |
| | | V143T, T150V, R183E, Q184E, N187K, G196S, D198N, A206D, T220S, S221Q, |
| | | E246N, A286P, N293Y, V304E, T329N, S342Q, V346I, Q350R, H351R, T358S, |
| | | A371T, A395K, A400P, A412G, T421R, A425N, K450T, E452S, E453P, G455S, |
| | | S460H, Q476D, D489E, N502S, S507C, S512Y |
| 320 | 21 | S25R, D37K, Q38A, S52G, N69P, I70M, A84D, A91P, T100S, D115T, A117T, |
| | | S132Q, Q133G, R183E, N187Q, D198H, A206T, S221Q, A286P, N293Y, V304E, |
| | | Q350R, H351R, A400P, E453P, S460H, D489E, N502S, S512Y |
| 321 | 21 | S25R, D37K, Q38A, S52G, T53V, W54Y, F58Y, T63Y, I70M, M81L, A84D, T88V, |
| | | A91P, D92S, T100S, T102S, D115T, A117K, T129N, T130K, S132Q, Q133G, |
| | | F141Y, V143T, T150V, R183E, Q184E, N187K, G196S, D198N, A206T, T220S, |
| | | S221Q, E246N, A286P, N293Y, V304E, T329N, S342Q, V346I, Q350R, H351R, |
| | | T358S, A371T, A395K, A400P, A412G, T421R, A425N, K450T, E452S, E453P, |
| | | G455S, S460H, Q476D, D489E, N502G, S507C, S512Y |
| 322 | 22 | N36S, A42G, K62S, V69A, K80S, N88S, T112S, E114S, D116P, K118D, I120S, |
| | | N134S, L138V, K149Q, E167D, S180G, K202A, S278C, K308H, V371I, K379Q, |
| | | F380H, N387C, S388A, F400C, I408M, T415C, V455P, S462E, L464M, V469K, |
| | | C484G, L514E, S562G, E573Y, K574P, Y576L, L770M, N835R, S839A, V842G, |
| | | Y844A, K852T, A857G, G887S, E946Q, E957D, E999A |
| 323 | 22 | N36S, A42G, K62S, V69A, K80S, N88S, T112S, E114S, D116P, K118D, I120S, |
| | | N134S, L138V, K149Q, E167D, S180G, K202A, S278C, K308H, V371I, K379Q, |
| | | F380H, N387C, S388A, F400C, I408M, T415C, V455P, S462E, L464M, V469K, |
| | | C484G, L514E, S562G, E573Y, K574P, Y576L, N835R, S839A, V842G, Y844A, |
| | | K852T, A857G, G887S, E946Q, E957D, E999A |
| 325 | 22 | N36S, F41C, A42G, K62S, V69A, S71G, T112S, E114S, D116P, I120K, N134S, |
| | | F175Y, S180G, K202A, V371I, K379Q, N387C, T415C, V455K, S462E, V469K, |
| | | C484G, L514E, S562G, E573Y, Y576L, L770M, N835R, V842G, Y844A, G887S, |
| | | E957D |
| 326 | 22 | N36S, A42G, K62S, V69A, K80S, N88S, T112S, E114S, D116P, K118D, I120S, |
| | | N134S, L138V, K149Q, E167D, F175Y, K176Q, S180G, K202A, S278C, K308H, |
| | | V371I, K379Q, F380H, N387C, S388A, F400C, I408M, T415C, D441K, Y446W, |
| | | V455P, S462E, L464M, V469K, C484G, L514E, F526V, S562G, E573Y, K574D, |
| | | Y576L, I581V, E768Q, S779T, N835R, S839A, V842G, Y844A, K852T, A857G, |
| | | N861K, G887S, Y916F, E946Q, E957D, E999A |
| 327 | 22 | N36S, F41C, A42G, K62S, V69A, E74S, K80S, N88S, T112S, E114S, D116P, |
| | | K118D, I120S, E121D, N134S, L138V, N148A, K149Q, I159V, E167D, K173R, |
| | | F175Y, K176Q, S180G, K202A, T216V, S278C, K308H, V371I, K379Q, F380H, |
| | | N387C, F400C, I408M, T415C, N423A, K438L, D441K, V442E, H445K, Y446W, |
| | | V455P, S462E, L464M, V469K, C484G, N512H, L514E, F526I, H536E, S562G, |
| | | E573Y, K574D, Y576L, V701S, E768Q, S779T, I816V, V832I, T833I, N835R, S839A, |
| | | V841A, V842G, Y844A, K852T, A857G, N861K, N863R, S865T, G887S, Y916F, |
| | | E946Q, E957D, E999A |
| 328 | 22 | N36S, F41C, A42G, A50V, K62S, V69A, E74S, K80S, N88S, T112S, E114S, D116P, |
| | | K118D, I120S, E121D, N134S, L138V, N148A, K149Q, I159V, E167D, K173R, |
| | | F175Y, S180G, K202A, T216V, S278C, K308H, V371I, K379Q, F380H, N387T, |
| | | S388A, F400C, I408M, T415C, N423A, K438L, D441K, V442E, H445K, Y446W, |
| | | V455P, S462E, L464M, V469K, C484G, L514E, F526I, H536E, S562G, E573Y, |
| | | K574D, Y576L, V701S, L770M, S779T, I816V, V832I, T833I, N835R, S839A, V841A, |
| | | V842G, Y844A, K852T, A857G, N861K, N863R, S865T, G887S, Y916F, E946Q, |
| | | E957D, E999A |
| 329 | 22 | N36G, F41C, A42G, A50V, K62S, V69A, E74S, K80S, N88S, T112S, E114S, D116P, |
| | | K118D, I120K, N134S, L138V, N148A, K149Q, E167D, K176Q, S180G, K202A, |
| | | S278C, K308H, V371I, K379Q, F380H, N387C, S388A, F400C, I408M, T415C, |
| | | K438L, D441K, H445K, Y446W, V455P, S462E, L464M, V469K, C484G, L514E, |
| | | F526I, S562G, E573Y, K574D, Y576L, L770M, S779T, T833I, N835R, S839A, |
| | | V842G, Y844A, K852T, A857G, N861K, S865T, G887S, Y916F, E946Q, E957D, |
| | | E999A |
| 330 | 22 | N36S, F41C, A42G, K62S, V69A, S71G, N88S, T112S, E114S, D116P, K118D, |
| | | I120S, N134S, L138V, K149Q, E167D, F175Y, S180G, K202A, S278C, V371I, |
| | | K379Q, F380H, N387C, S388A, F400C, I408M, T415C, V455P, S462E, L464M, |
| | | V469K, C484G, L514E, S562G, E573Y, K574P, Y576L, N835R, S839A, V842G, |
| | | Y844A, A857G, G887S, E957D |
| 331 | 22 | N36S, A42G, K62S, V69A, S71G, N88S, T112S, E114S, D116P, K118D, I120S, |
| | | N134S, K149Q, E167D, F175Y, S180G, K202A, S278C, V371I, K379Q, F380H, |
| | | N387C, S388A, F400C, I408M, T415C, V455P, S462E, L464M, V469K, C484G, |
| | | L514E, S562G, E573Y, K574P, Y576L, L770M, N835R, S839A, V842G, Y844A, |
| | | A857G, G887S, E957D |
| 332 | 22 | N36S, F41C, A42G, A50V, K62S, V69A, S71G, E74S, K80S, N88S, T112S, E114S, |
| | | D116P, K118D, I120K, N134S, L138V, N148A, K149Q, E167D, K176Q, S180G, |
| | | K202A, S278C, K308H, V371I, K379Q, F380H, N387C, S388A, F400C, I408M, |
| | | T415C, K438L, D441K, H445K, Y446W, V455P, S462E, L464M, V469K, C484G, |
| | | L514E, F526I, S562G, E573Y, K574D, Y576L, E768Q, S779T, T833I, N835R, |
| | | S839A, V842G, Y844A, K852T, A857G, N861K, S865T, G887S, Y916F, E946Q, |
| | | E957D, E999A |
| 333 | 22 | N36S, F41C, A42G, K62S, V69A, S71G, T112S, E114S, D116P, I120S, N134S, |
| | | S180G, K202A, K379Q, N387C, T415C, V455K, S462E, V469K, C484G, L514E, |
| | | S562G, E573Y, Y576L, N835R, V842G, G887S |
| 334 | 22 | N36G, A42G, K62S, V69A, T112S, E114S, D116P, I120S, N134S, F175Y, S180G, |
| | | K202A, V371I, K379Q, N387C, T415C, V455E, S462E, V469K, C484G, L514E, |
| | | S562G, E573Y, Y576L, N835R, V842G, Y844A, G887S, E957D |
| 335 | 22 | N36S, F41C, A42G, A50V, K62S, V69A, K80S, N88S, T112S, E114S, D116P, |
| | | K118D, I120S, N134S, L138V, K149Q, E167D, K176Q, S180G, K202A, S278C, |
| | | K308H, V371I, K379Q, F380H, N387T, S388A, F400C, I408M, T415C, D441K, |
| | | Y446W, V455P, S462E, L464M, V469K, C484G, N512H, L514E, F526V, S562G, |
| | | E573Y, K574D, Y576L, E768Q, S779T, N835R, S839A, V842G, Y844A, K852T, |
| | | A857G, N861K, G887S, Y916F, E946Q, E957D, E999A |
| 337 | 23 | G22T, A28R, N37G, G44A, K51Y, F52Y, S109A, N111E, R132H, T136H, Q139S, |
| | | A140K, R146D |
| 338 | 23 | G22T, A28R, N37G, G44A, K51Y, F52H, S109A, R132H, T136R, Q139S, A140K, |
| | | R146D |
| 339 | 23 | K51Y, R132H, Q139S |
| 340 | 23 | N37G, G44A, K51Y, F52H, S109A, R132H, Q139S |
| 341 | 23 | G22T, A28R, N37G, R39K, G44A, K51Y, F52Y, S90A, S109A, N111E, V127D, |
| | | R132H, T136H, Q139S, A140K, R146D |
| 342 | 23 | G44A, K51Y, S109A, R132H, Q139S |
| 343 | 23 | F21Y, A28R, N37G, R39K, G44A, K51Y, F52Y, S90A, R91K, S109A, N111E, |
| | | V127D, R132H, T136H, Q139S, A140K, R146D |
| 344 | 23 | F21Y, A28R, N37G, R39K, K51Y, F52Y, S90A, R91K, S104D, S109A, N111E, |
| | | V127D, R132H, T136H, Q139S, A140K, R146D |
| 345 | 23 | G22T, A28K, N37G, R39K, G44A, K51Y, F52Y, S90A, S109A, N111E, V127D, |
| | | R132H, T136K, Q139S, A140K, R146D |
| 346 | 24 | H32S, T77S, A84C, Q93C, H99N, S100N, D103C, I121T, T137S, K153E, D179N, |
| | | L215D, R219A, D221S, N227C, Y235M, M242Q, N252H, L253T, M257G, K259A, |
| | | Q261G, F276N, I283V, Q310H, A324C, Q332C, T361A, N367A, T372V, M373L |
| 347 | 24 | V24S, H32S, D49A, T77S, A84C, Q91A, Q93C, L98Q, H99N, S100N, D103C, |
| | | K118N, I121T, T137S, K153E, A161H, K168Q, V178I, D179N, S214N, L215D, |
| | | R219N, D221S, N227C, Q229H, Y235M, L236F, M242Q, F250I, N252H, L253T, |
| | | M257G, K259D, Q261G, L272V, F273W, H274Y, F276N, Q310H, S314N, A324C, |
| | | Q332C, T361A, T372A, Q378A |
| 348 | 24 | V24Q, H32S, T77S, A84C, Q91A, Q93C, L98Q, H99N, S100N, D103C, I121T, |
| | | T137S, K153E, A161H, K168Q, D179N, L215D, R219A, D221S, N227C, Q229H, |
| | | Y235M, M242Q, F250I, N252H, L253T, M257G, K259D, Q261G, L272V, F273W, |
| | | H274Y, F276N, I283V, Q310H, A324C, Q332C, T361A, N367A, T372A, Q378A |
| 349 | 24 | V24S, D49A, T77S, A84C, Q91A, Q93C, L98K, H99N, S100N, D103C, T137S, |
| | | V140A, V148I, V178I, D179N, Y213N, L215D, I216F, N227C, Q229H, N252H, |
| | | L253S, M257G, K259A, N275Q, F276N, A291I, I307L, A324C, Q332C, S339N, |
| | | T361A, N367M, T372V, Q378A |
| 350 | 24 | A84C, Q93C, D103C, N227C, A324C, Q332C, T361A, N367A |
| 351 | 24 | H32S, T77S, A84C, Q93C, H99D, S100N, D103C, T137S, L215D, D217Y, R219A, |
| | | N227C, Y235L, M242Q, N252H, L253T, M257G, K259A, Q261G, F276N, Q310H, |
| | | A324C, Q332C, T361A, N367A, T372V |
| 352 | 24 | H32S, T77S, A84C, Q93C, H99D, S100N, D103C, T137S, V140A, L215D, D217Y, |
| | | R219A, N227C, Y235L, M242Q, N252H, L253T, M257G, Q261G, F276N, Q310H, |
| | | A324C, Q332C, T361A, N367A, T372V |
| 353 | 24 | H32S, T77S, A84C, Q91A, Q93C, H99N, S100N, D103C, I121T, T137S, V140A, |
| | | K153E, A161H, D179N, L215D, R219A, D221S, N227C, Q229H, Y235M, M242Q, |
| | | F250V, N252H, L253T, M257G, K259D, Q261G, L272V, F273W, F276N, I283V, |
| | | Q310H, A324C, Q332C, T361A, N367A, T372A |
| 354 | 24 | H32S, T77S, A84C, Q93C, H99D, S100N, D103C, I121T, T137S, V140A, L215D, |
| | | R219A, D221S, N227C, Y235M, M242Q, N252H, L253T, M257G, K259D, Q261G, |
| | | F276N, Q310H, A324C, Q332C, T361A, N367A, T372V |
| 355 | 24 | T77S, A84C, Q93C, H99N, S100N, D103C, T137S, V140A, D179N, Y213N, N227C, |
| | | Q229H, N252H, L253S, M257G, F273W, F276N, A324C, Q332C, T361A, N367A |
| 356 | 24 | V24S, H32S, D49A, T77S, A84C, Q91A, Q93C, L98Q, H99N, S100N, D103C, |
| | | K118N, I121T, T137S, K153E, A161H, K168Q, V178I, D179N, S214N, L215D, |
| | | R219N, D221S, N227C, Q229H, Y235M, L236F, M242Q, F250I, N252H, L253T, |
| | | M257G, K259A, Q261G, L272V, F273W, H274Y, F276N, I283V, Q310H, S314N, |
| | | A324C, Q332C, T361A, T372A, Q378A |
| 357 | 24 | H32S, T77S, A84C, Q93C, H99N, S100N, D103C, T137S, V140A, K153E, D179N, |
| | | L215D, R219A, D221S, N227C, Y235M, M242Q, N252H, L253T, M257G, K259A, |
| | | Q261G, F276N, I283V, Q310H, A324C, Q332C, T361A, N367A, T372V, M373L |
| 358 | 24 | D49A, T77S, A84C, Q93C, L98K, H99N, S100N, D103C, T137S, V140A, V148I, |
| | | D179N, Y213N, L215D, N227C, Q229H, N252H, L253S, M257G, K259A, F273W, |
| | | F276N, A324C, Q332C, T361A, N367A, T372V |
| 359 | 24 | V24S, H32S, D49A, N58S, T77S, A84C, Q91A, Q93C, L98Q, H99N, S100N, D103C, |
| | | K118N, I121T, T137S, K153E, A161H, V162I, K168Q, V178I, D179N, Y213N, |
| | | S214N, L215D, R219N, D221S, N227C, Q229H, Y235M, L236F, M242Q, F250I, |
| | | N252H, L253T, M257G, K259D, Q261G, L272V, F273W, H274Y, F276N, I283V, |
| | | A291L, Q310H, S313N, S314N, A324C, Q332C, T361A, T372A, Q378A |
| 360 | 24 | V24S, D49A, T77S, A84C, Q91A, Q93C, L98K, H99N, S100N, D103C, T137S, |
| | | V140A, V148I, V178I, D179N, Y213N, L215D, N227C, Q229H, N252H, L253S, |
| | | M257G, K259A, F273W, N275Q, F276N, A291I, I307L, A324C, Q332C, S339N, |
| | | T361A, N367M, T372V, Q378A |
| 361 | 24 | T77S, A84C, Q93C, H99N, S100N, D103C, T137S, V140A, D179N, Y213N, N227C, |
| | | N252H, L253S, M257G, F276N, A324C, Q332C, T361A, N367A |
| 362 | 24 | V24Q, H32S, T77S, A84C, Q91A, Q93C, L98Q, H99N, S100N, D103C, I121T, |
| | | T137S, K153E, A161H, K168Q, D179N, L215D, R219A, D221S, N227C, Q229H, |
| | | Y235M, M242Q, F250I, N252H, L253T, M257G, K259A, Q261G, L272V, F273W, |
| | | H274Y, F276N, I283V, Q310H, A324C, Q332C, T361A, N367A, T372A, Q378A |
| 363 | 24 | V24T, D49A, N58S, T77S, A84C, Q91A, Q93C, L98K, H99N, S100N, D103C, |
| | | T137S, V140A, V148I, A161H, V162I, I165L, V178I, D179N, Y213N, L215D, N227C, |
| | | Q229H, N252H, L253S, M257G, K259A, N275S, F276N, D277N, A291I, I307L, |
| | | A324C, Q332C, S339N, T361A, Q370E, T372V, Q378A |
| 364 | 24 | V24S, D49A, N58S, T77S, A84C, Q91A, Q93C, L98K, H99N, S100N, D103C, |
| | | T137S, V140A, V148I, A161H, V162I, I165L, V178I, D179N, Y213N, L215D, I216F, |
| | | N227C, Q229H, N252H, L253S, M257G, K259A, F273W, N275Q, F276N, D277N, |
| | | A291I, I307L, A324C, Q332C, S339N, T361A, N367R, Q370E, T372V, Q378A |
| 365 | 24 | V24S, D49A, T77S, A84C, Q91A, Q93C, L98K, H99N, S100N, D103C, T137S, |
| | | V140A, V148I, V178I, D179N, Y213N, L215D, N227C, Q229H, N252H, L253S, |
| | | M257G, K259A, F273W, N275Q, F276N, A324C, Q332C, T361A, N367A, T372V, |
| | | Q378A |
| 366 | 24 | V24S, H32S, D49A, N58S, T77S, A84C, Q91A, Q93C, L98Q, H99N, S100N, D103C, |
| | | K118N, I121T, T137S, K153E, A161H, V162I, K168Q, V178I, D179N, Y213N, |
| | | S214N, L215D, R219N, D221S, N227C, Q229H, Y235M, L236F, M242Q, F250I, |
| | | N252H, L253T, M257G, K259A, Q261G, L272V, F273W, H274Y, F276N, I283V, |
| | | A291L, Q310H, S313N, S314N, A324C, Q332C, T361A, T372A, Q378A |
| 367 | 24 | H32S, T77S, A84C, Q93C, H99D, S100N, D103C, I121T, T137S, V140A, L215D, |
| | | R219A, D221S, N227C, Y235M, M242Q, N252H, L253T, M257G, K259A, Q261G, |
| | | F276N, Q310H, A324C, Q332C, T361A, N367A, T372V |
| 368 | 24 | H32S, T77S, A84C, Q91A, Q93C, H99N, S100N, D103C, T137S, A161H, D179N, |
| | | L215D, R219A, D221S, N227C, Q229H, Y235M, M242Q, F250V, N252H, L253T, |
| | | M257G, K259A, Q261G, L272V, F273W, F276N, I283V, Q310H, A324C, Q332C, |
| | | T361A, N367A, T372A |
| 369 | 24 | T77S, A84C, Q93C, H99N, S100N, D103C, T137S, V140A, D179N, Y213N, L215D, |
| | | N227C, Q229H, N252H, L253S, M257G, K259A, F273W, F276N, A324C, Q332C, |
| | | T361A, N367A, I368Q |
| 370 | 25 | P26G, E27D, S37M, A47P, T48P, D57H, A58P, T61Q, K65A, L66A, V89R, A90Q, |
| | | A95Q, P100C, S102D, D110N, E113Q, G119A, E120A, L126A, A131C, T133R, |
| | | T136Y, T140M, S141G, S142T, P143M, V154G, Q156C, A160K, N161Q, S168K, |
| | | S173G, A175Q, D176A, F177E, T178H, G179E, H180R, T183A, F185Y, R190K, |
| | | L201C, Q206C, L235R, T240A, P252Q, E253D, G255A, S262E, H263Q, N266A, |
| | | T267R, L269M, S270E, Y277H, Q280F, T282A, E284Y, R287A, S288A, T291G, |
| | | M298Q, K308N, I322M, A335M, E337G, L342F, E354A, R362D, L363K, S364A, |
| | | N366Y, S367K, I370V, S373A, F376Y, K385A, N391A, T392Q, T400C, G417Q |
| 371 | 25 | P26G, S37M, T48P, K65A, L66A, V89R, A90Q, A95Q, P100C, S102A, D110N, |
| | | E120A, L126A, A131C, T140L, S141G, S142T, P143M, V154G, Q156C, N161Q, |
| | | S168K, S173G, A175Q, G179A, T183E, F185Y, R190K, L201C, Q206C, T240A, |
| | | P252Q, G255A, S262A, H263Q, T267R, Y277H, T282A, E284Y, R287A, S288A, |
| | | A335M, L342F, E354K, R362D, S364A, N366Y, S367K, F376Y, K385A, T392Q, |
| | | T400C, G417Q |
| 372 | 25 | P26G, S37M, T48P, K65A, L88W, V89M, A90Q, A95Q, P100C, S102A, D110N, |
| | | E120A, L126A, A131C, T140L, S141G, S142T, P143M, V154G, Q156C, S168K, |
| | | S173G, A175Q, G179R, F185Y, R190K, L201C, Q206C, T240A, P252Q, G255A, |
| | | S262A, H263Q, Y277H, T282A, E284Y, S288A, A335M, L342F, E354K, R362D, |
| | | N366Y, K385A, T400C, G417Q |
| 373 | 25 | P100C, A131C, Q156C, L201C, Q206C, T400C |
| 374 | 25 | P26G, S37M, K65A, L88W, V89M, A95Q, P100C, S102P, D110N, E120A, L126A, |
| | | A131C, T140M, S142T, P143M, V154G, Q156C, S173G, A175Q, G179R, R190K, |
| | | L201C, Q206C, T240A, P252Q, S262A, H263Q, Y277H, T282A, E284Y, S288A, |
| | | E354K, R362D, N366Y, T400C, G417Q |
| 375 | 25 | P26G, S37M, T48P, D57S, A58P, K65A, L66A, V89R, A90Q, A95Q, P100C, S102A, |
| | | D110N, G119A, E120A, L126A, A131C, T140L, S141G, S142T, P143M, V154G, |
| | | Q156C, A160K, N161Q, S168K, S173G, A175Q, T178H, G179E, H180R, T183A, |
| | | F185Y, R190K, L201C, Q206C, T240A, P252Q, G255A, S262A, H263Q, N266A, |
| | | T267R, Y277H, Q280F, T282A, E284Y, R287A, S288A, T291G, M298Q, K308N, |
| | | A335M, E337G, L342F, E354A, R362D, S364A, N366Y, S367K, F376Y, K385A, |
| | | N391A, T392Q, T400C, G417Q |
| 376 | 25 | P26G, S37M, L88W, V89M, A95Q, P100C, S102P, D110N, E120A, L126A, A131C, |
| | | T140M, P143M, V154G, Q156C, S173G, A175Q, G179R, R190K, L201C, Q206C, |
| | | T240A, P252Q, S262A, H263Q, Y277H, T282A, E284Y, R362D, N366Y, T400C, |
| | | G417Q |
| 377 | 25 | P26G, S37M, T48P, K65A, L88W, V89M, A90Q, A95Q, P100C, S102A, D110N, |
| | | E120A, L126A, A131C, T140L, S141G, S142K, P143M, V154G, Q156C, S168K, |
| | | S173G, A175Q, G179R, F185Y, R190K, L201C, Q206C, T240A, P252Q, G255A, |
| | | S262A, H263Q, Y277H, T282A, E284Y, S288A, A335M, L342F, E354K, R362D, |
| | | N366Y, K385A, T400C, G417Q |
| 378 | 25 | P26G, E27D, S37M, A47P, T48P, L50T, D53R, V54M, D57R, A58P, T61Q, K65A, |
| | | L66A, Q84F, L88M, V89R, A90Q, D91Q, A95Q, P100C, S102D, I108W, D110N, |
| | | E113Q, G119A, E120A, F122Y, L126A, D129G, A131C, I132V, T133R, T140M, |
| | | S141G, S142T, P143M, V154G, Q156C, A160Q, N161Q, V162A, D164A, I166V, |
| | | S168K, S173G, A175Q, D176A, F177E, T178H, G179E, H180R, T183A, F185Y, |
| | | E187Q, R190K, L201C, Q206C, S234A, L235R, T240A, P252Q, E253D, G255A, |
| | | T260H, S262E, H263Q, N266A, T267R, S270A, Y277H, Q280F, T282A, E284Y, |
| | | R287A, S288A, D295Q, L296F, M298Q, A299T, K308N, Q309N, T314A, L315H, |
| | | L320G, I322M, A335M, E337G, L342F, E354A, R362D, L363K, S364A, D365A, |
| | | N366Y, S367K, I370V, S373A, F376Y, M382L, D384Q, K385A, N391A, T392Q, |
| | | T400C, A402Q, A416D, G417R |
| 379 | 25 | P26G, E27D, S37M, A47P, T48P, D57R, A58P, T61Q, K65A, L66A, V89R, A90Q, |
| | | A95Q, P100C, S102D, D110N, E113Q, G119A, E120A, L126A, A131C, T133R, |
| | | T136Y, T140M, S141G, S142T, P143M, V154G, Q156C, A160K, N161Q, T163V, |
| | | S168K, S173G, A175Q, D176A, F177E, T178H, G179E, H180R, T183A, F185Y, |
| | | R190K, L201C, Q206C, L235R, T240A, P252Q, E253D, G255A, S262E, H263Q, |
| | | N266A, T267R, L269M, Y277H, Q280F, T282A, E284Y, R287A, S288A, P292Y, |
| | | M298Q, K308N, I322M, A335M, E337G, L342F, E354A, R362D, L363K, S364A, |
| | | N366Y, S367K, I370V, S373A, F376Y, K385A, N391A, T392Q, T400C, G417Q |
| 380 | 25 | P26G, E27D, S37M, A47P, T48P, L50T, D53R, V54M, D57R, A58P, T61Q, K65A, |
| | | L66A, Q84F, L88M, V89R, A90Q, D91Q, A95Q, P100C, S102D, D110N, E113Q, |
| | | G119A, E120A, F122Y, L126A, D129G, A131C, I132V, T133R, T140M, S141G, |
| | | S142T, P143M, V154G, Q156C, A160Q, N161Q, V162A, D164A, I166V, S168K, |
| | | S173G, A175Q, D176A, F177E, T178H, G179E, H180R, T183A, F185Y, E187Q, |
| | | R190K, L201C, Q206C, S234A, L235R, T240A, P252Q, E253D, G255A, T260H, |
| | | S262E, H263Q, N266A, T267R, S270A, Y277H, Q280F, T282A, E284Y, R287A, |
| | | S288A, T291G, D295R, L296F, M298Q, A299T, K308N, Q309N, T314A, L320G, |
| | | I322M, A335M, E337G, L342F, E354A, R362D, L363K, S364A, D365A, N366Y, |
| | | S367K, I370V, S373A, F376Y, M382L, D384Q, K385A, N391A, T392Q, T400C, |
| | | A402Q, A416D, G417R |
| 381 | 25 | P26G, S37M, L88W, V89M, A95Q, P100C, S102P, D110N, E120A, L126A, A131C, |
| | | T140M, P143M, V154G, Q156C, S173G, A175Q, G179R, R190K, L201C, Q206C, |
| | | T240A, P252Q, S262A, H263Q, Y277H, T282A, E284Y, L315H, R362D, N366Y, |
| | | T400C, G417Q |
| 382 | 25 | P26G, S37M, T48P, D57S, A58P, K65A, L66A, V89R, A90Q, A95Q, P100C, S102A, |
| | | D110N, G119A, E120A, L126A, A131C, T140M, S141G, S142K, P143M, V154G, |
| | | Q156C, A160K, N161Q, S168K, S173G, A175Q, T178H, G179E, H180R, T183A, |
| | | F185Y, R190K, L201C, Q206C, T240A, P252Q, G255A, S262A, H263Q, N266A, |
| | | T267R, Y277H, Q280F, T282A, E284Y, R287A, S288A, T291G, M298Q, K308N, |
| | | A335M, E337G, L342F, E354A, R362D, S364A, N366Y, S367K, F376Y, K385A, |
| | | N391A, T392Q, T400C, G417Q |
| 383 | 25 | P26G, S37M, T48P, K65A, L66A, V89R, A90Q, A95Q, P100C, S102A, D110N, |
| | | E120A, L126A, A131C, T140M, S141G, S142T, P143M, V154G, Q156C, N161Q, |
| | | S168K, S173G, A175Q, G179E, T183E, F185Y, R190K, L201C, Q206C, T240A, |
| | | P252Q, G255A, S262E, H263Q, T267R, Y277H, T282A, E284Y, R287A, S288A, |
| | | A335M, E354K, R362D, S364A, N366Y, S367K, F376Y, K385A, T392Q, T400C, |
| | | G417Q |
| 384 | 26 | T43V, S50M, R65D, V67W, L79P, E87A, I90M, R91A, F96Y, R98H, K100W, I106L, |
| | | N111G, P113C, D123N, G132A, E133Q, L139A, G144C, A156M, K164I, I167D, |
| | | S169C, N173E, E182R, Q184H, P186S, L195A, T203E, Q214C, D219C, L249I, |
| | | Y261Q, P266A, K267D, N272R, E278Q, T288A, A310Y, N313G, P329L, F335V, |
| | | N345A, S347A, N351G, A375R, K380T, S384R, Q411R, K413N, A422P, E423D, |
| | | P429S, T432S, M434L, Q437H, G442A |
| 385 | 26 | T43L, S50M, R65D, V67W, L79P, E87A, I90M, R91A, F96Y, R98H, K100W, I106L, |
| | | Q109A, N111G, P113C, D123N, G132A, E133A, L139A, G144C, N152D, A156M, |
| | | K164I, I167D, S169C, N173E, E182R, Q184H, P186S, L195A, T203E, Q214C, |
| | | D219C, I234L, I247L, G248A, Y261Q, P266Q, K267D, N272R, E278Q, A281K, |
| | | T288A, A310Y, N313G, P329H, F335M, I336L, N345A, S347A, N351G, A375R, |
| | | S378A, K380T, S384R, Q411R, K413N, A422P, E423D, P429E, T432S, M434L, |
| | | Q437H, G442A |
| 386 | 26 | S39P, G40D, T43V, S50M, M60S, M64L, R65Q, D66K, V67W, L79P, I82L, E87A, |
| | | I90M, R91A, V92Q, F96Y, E99R, K100W, I106L, Q109A, N111G, P113C, S117T, |
| | | D123N, L129E, G132A, E133Q, L139A, G144C, N152D, A156M, K164I, I167D, |
| | | S169C, N173E, I175A, E182R, Q184G, N185G, P186S, L195A, N202A, T203Q, |
| | | Q214C, D219C, I234L, I247L, G248A, E260Q, Y261Q, P266Q, K267D, N272R, |
| | | S275D, E278Q, A281K, K284A, T288A, A294N, I299V, P306N, L308M, A310Y, |
| | | I311L, N313G, N318Q, A319P, I327D, S328A, P329Q, F335L, I336L, N345A, |
| | | S347A, N351G, S353H, T355N, L374W, A375R, S378A, K380T, S384R, Q411R, |
| | | K413N, A422P, E423D, P429E, T432S, M434L, Q437E, S438A, V439L, G442A |
| 387 | 26 | T43V, S50M, R65Q, L79P, E87A, I90M, R91A, F96Y, R98H, K100W, I106L, N111G, |
| | | P113C, G132A, E133Q, L139A, G144C, A156M, K164I, I167D, S169C, N173E, |
| | | E182R, P186S, L195A, Q214C, D219C, Y261Q, P266Q, K267D, E278Q, A281K, |
| | | A310Y, N313G, P329H, F335M, K380T, S384R, Q411R, K413N, A422P, E423D, |
| | | P429S, T432S, M434L, Q437H, G442A |
| 388 | 26 | T43V, S50M, R65D, V67W, L79P, E87A, R91A, F96Y, R98H, K100W, I106L, |
| | | N111G, P113C, D123N, G132A, E133Q, L139A, G144C, A156M, K164I, I167D, |
| | | S169C, N173E, E182R, Q184H, P186S, L195A, T203E, Q214C, D219C, L249I, |
| | | Y261Q, P266Q, K267D, N272R, E278Q, T288A, A310Y, N313G, P329H, F335M, |
| | | N345A, S347A, N351G, A375R, K380T, S384R, Q411R, K413N, A422P, E423D, |
| | | P429S, T432S, M434L, Q437H, G442A |
| 389 | 26 | S39P, T43V, S50M, M60S, R65D, D66K, V67W, L79P, I82L, E87A, I90M, R91A, |
| | | F96Y, R98H, K100W, I106L, Q109A, N111G, P113C, D123N, G132A, E133Q, |
| | | L139A, G144C, N152D, A156M, K164I, I167D, S169C, N173E, I175A, E182R, |
| | | Q184G, N185G, P186S, L195A, N202D, T203E, Q214C, D219C, I234L, G248A, |
| | | L249I, E260Q, Y261Q, P266Q, K267D, N272R, S275D, E278Q, T288A, A294N, |
| | | A310Y, N313G, N318H, S328A, P329Q, N331A, F335M, I336L, N345A, S347A, |
| | | N351G, T355N, L374W, A375R, S378A, K380T, Y382W, S384R, Q411R, K413N, |
| | | A422P, E423D, P429E, T432S, M434L, Q437E, G442A |
| 390 | 26 | T43V, S50M, R65D, D66K, V67W, L79P, I82L, E87A, I90M, R91A, F96Y, R98H, |
| | | K100W, I106L, Q109A, N111G, P113C, D123N, G132A, E133Q, L139A, G144C, |
| | | N152D, A156M, K164I, I167D, S169C, N173E, E182R, Q184H, N185G, P186S, |
| | | L195A, N202D, T203E, Q214C, D219C, I234L, G248A, L249I, E260Q, Y261Q, |
| | | P266A, K267N, N272R, S275D, E278Q, T288A, A294N, A310Y, N313G, N318H, |
| | | S328A, P329L, N331A, F335M, I336L, N345A, S347A, N351G, T355N, L374W, |
| | | A375R, S378A, K380T, S384R, Q411R, K413N, A422P, E423D, P429E, T432S, |
| | | M434L, Q437H, G442A |
| 391 | 26 | P113C, G144C, S169C, Q214C, D219C, Q411R, K413N, A422P, E423D, P429S, |
| | | T432S, M434L, Q437H, G442A |
| 392 | 26 | T43V, S50M, R65D, D66K, V67W, L79P, I82L, E87A, I90M, R91A, F96Y, R98H, |
| | | K100W, I106L, Q109A, N111G, P113C, D123N, G132A, E133Q, L139A, G144C, |
| | | N152D, A156M, K164I, I167D, S169C, N173E, E182R, N185G, P186S, L195A, |
| | | N202D, T203E, Q214C, D219C, I234L, G248A, L249I, E260Q, Y261Q, P266Q, |
| | | K267N, N272R, S275D, E278Q, T288A, A294N, A310Y, I311L, N313G, S328A, |
| | | P329L, F335M, I336L, N345A, S347A, N351G, S353H, T355N, L374W, A375R, |
| | | S378A, K380T, S384R, Q411R, K413N, A422P, E423D, P429E, T432S, M434L, |
| | | Q437H, G442A |
| 393 | 26 | T43V, S50M, R65D, L79P, E87A, I90M, R91A, F96Y, R98H, K100W, I106L, N111G, |
| | | P113C, G132A, E133Q, L139A, G144C, A156M, K164I, I167D, S169C, N173E, |
| | | E182R, P186S, L195A, Q214C, D219C, I247L, Y261Q, P266A, K267N, E278Q, |
| | | A281K, A310Y, N313G, P329H, F335M, K380T, S384R, Q411R, K413N, A422P, |
| | | E423D, P429S, T432S, M434L, Q437H, G442A |
| 394 | 26 | S39P, T43L, S50M, M60S, R65Q, D66K, V67W, L79P, I82L, E87A, I90M, R91A, |
| | | V92A, F96Y, R98H, K100W, I106L, Q109A, N111G, P113C, D123N, L129E, G132A, |
| | | E133A, L139A, G144C, N152D, A156M, K164I, I167D, S169C, N173E, I175A, |
| | | E182R, Q184G, N185G, P186S, L195A, N202E, T203E, Q214C, D219C, I234L, |
| | | I247L, G248A, E260Q, Y261Q, P266Q, K267D, N272R, S275D, E278Q, T288A, |
| | | A294N, A310Y, I311L, N313G, A319P, I327D, S328A, P329Q, F335L, I336L, N345A, |
| | | S347A, N351G, S353H, T355N, L374W, A375R, S378A, K380T, S384R, Q411R, |
| | | K413N, A422P, E423D, P429E, T432S, M434L, Q437E, G442A |
| 395 | 26 | T43V, S50M, R65D, V67W, L79P, E87A, I90M, R91A, F96Y, R98H, K100W, I106L, |
| | | Q109A, N111G, P113C, D123N, G132A, E133Q, L139A, G144C, N152D, A156M, |
| | | K164I, I167D, S169C, N173E, E182R, Q184H, P186S, L195A, T203E, Q214C, |
| | | D219C, I234L, I247L, G248A, Y261Q, P266Q, K267N, N272R, E278Q, A281K, |
| | | T288A, A310Y, N313G, P329H, N331A, F335M, I336L, N345A, S347A, N351G, |
| | | A375R, S378A, K380T, S384R, Q411R, K413N, A422P, E423D, P429E, T432S, |
| | | M434L, Q437H, G442A |
| 396 | 26 | S39P, T43V, S50M, M60S, R65Q, D66K, V67W, L79P, I82L, E87A, I90M, R91A, |
| | | V92A, F96Y, R98H, K100W, I106L, Q109A, N111G, P113C, D123N, L129E, G132A, |
| | | E133Q, L139A, G144C, N152D, A156M, K164I, I167D, S169C, N173E, I175A, |
| | | E182R, Q184G, N185G, P186S, L195A, N202A, T203Q, Q214C, D219C, I234L, |
| | | I247L, G248A, E260Q, Y261Q, P266Q, K267N, N272R, S275D, E278Q, T288A, |
| | | A294N, A310Y, I311L, N313G, A319P, I327D, S328A, P329Q, F335L, I336L, N345A, |
| | | S347A, N351G, T355N, L374W, A375R, S378A, K380T, S384R, Q411R, K413N, |
| | | A422P, E423D, P429E, T432S, M434L, Q437E, G442A |
| 397 | 26 | S39P, T43V, S50M, M60S, R65Q, D66K, V67W, L79P, I82L, E87A, I90M, R91A, |
| | | F96Y, R98H, K100W, I106L, Q109A, N111G, P113C, D123N, L129E, G132A, |
| | | E133Q, L139A, G144C, N152D, A156M, K164I, I167D, S169C, N173Q, I175A, |
| | | E182R, Q184G, N185G, P186S, L195A, N202D, T203E, Q214C, D219C, I234L, |
| | | G248A, E260Q, Y261Q, P266Q, K267N, N272R, S275D, E278Q, T288A, A294N, |
| | | A310Y, I311L, N313G, S328A, P329Q, F335M, I336L, N345A, S347A, N351G, |
| | | T355N, L374W, A375R, S378A, K380T, Y382W, S384R, Q411R, K413N, A422P, |
| | | E423D, P429E, T432S, M434L, Q437E, G442A |
| 398 | 26 | S39A, G40D, T43V, S50M, M60S, M64L, R65Q, D66K, V67W, L79P, I82L, E87A, |
| | | Q88H, I90M, R91A, V92Q, F96Y, R98H, E99R, K100W, I106L, Q109A, N111G, |
| | | P113C, S117T, D123N, V124I, L129E, G132A, E133Q, L139A, G144C, N152D, |
| | | A156M, K164I, I167D, S169C, N173E, I175A, E182R, Q184G, N185G, P186S, |
| | | L195A, N202A, T203Q, Q214C, D219C, I234L, I247L, G248A, E260Q, Y261Q, |
| | | P266Q, K267D, N272R, S275D, E278Q, A281K, K284A, T288A, A294N, I299V, |
| | | P306N, L308M, A310Y, I311L, N313G, N318Q, I327D, S328A, P329Q, F335L, |
| | | I336L, N345A, S347A, N351G, T355N, L374W, A375R, S378A, K380T, S384R, |
| | | Q411R, K413N, A422P, E423D, P429E, T432S, M434L, Q437E, S438A, V439L, |
| | | G442A |
| 399 | 27 | T36C, A43E, S48Q, S51C, A71L, D73S, T78I, D115G, S122G, V140L, K146T, |
| | | I148L, N168D, D179S, G187A, G195Y, I210C, S217H, S226C, K245Q, S250T, |
| | | S252N, D256N, A258T, I266N, K268G, E269G, D272S, E275N, Q282I, I286C, |
| | | S301G, V315F, G319N, L324V, E325A, D326S, Q330N, A356T, D359K |
| 400 | 27 | T36C, A43E, S51C, V140L, K146S, I148L, I210C, S217N, S226C, K245Q, A258T, |
| | | I266N, K268G, E269G, D272S, Q282I, S301G, G319N, L324V, D326S, A356T, |
| | | D359K |
| 401 | 27 | T36C, A40H, E42D, A43E, E45K, S46A, I47Q, S48Q, S51C, S69K, A71L, D73S, |
| | | T78I, M106Q, D115G, S122G, D139K, V140L, F145I, K146T, I148L, I158V, V165L, |
| | | N168D, D179S, G187A, I210C, I212V, S217N, S226C, K245Q, S250T, S252C, |
| | | D256N, A258D, I266N, K268G, E269G, D272S, E275N, I276F, Q282I, I286C, |
| | | S301G, I310V, D314N, V315F, G319N, L324V, E325A, D326S, Q330N, D343N, |
| | | S352T, A356T, D358T, D359K |
| 402 | 27 | T36C, S51C, I210C, S226C, A356T, D359K |
| 403 | 27 | T36C, A43E, S48Q, S51C, A71L, T78I, S122G, V140L, K146T, I148L, G195Y, |
| | | I210C, S217N, S226C, K245Q, D256N, A258D, I266N, K268G, E269G, D272S, |
| | | E275N, Q282I, S301G, G319N, L324V, D326S, A356T, D359K |
| 404 | 27 | T36C, A43E, S48Q, S51C, A71L, D73S, T78I, D115G, S122G, V140L, K146T, |
| | | I148L, N168D, D179S, G187A, G195Y, I210C, S217N, S226C, K245Q, S250T, |
| | | S252N, D256N, A258D, I266N, K268G, E269G, D272S, E275N, Q282I, I286C, |
| | | S301G, V315F, G319N, L324V, E325A, D326S, Q330N, A356T, D359K |
| 405 | 27 | T36C, A43E, S51C, V140L, K146T, I148L, I210C, S217N, S226C, K245Q, A258T, |
| | | I266N, K268G, E269G, D272S, Q282I, S301G, G319N, L324V, D326S, A356T, |
| | | D359K |
| 406 | 27 | T36C, E42D, A43E, S46A, I47Q, S48Q, S51C, A71L, D73S, T78I, D115G, S122G, |
| | | V140L, K146T, I148L, N168D, D179S, G187A, G195Y, I210C, S217N, S226C, |
| | | K245Q, S250T, S252C, D256N, A258T, I266N, K268G, E269G, D272S, E275N, |
| | | Q282I, I286C, S301G, I310V, V315F, G319N, L324V, E325A, D326S, Q330N, |
| | | D343N, S352T, A356T, D359K |
| 407 | 27 | T36C, A43E, S48Q, S51C, A71L, D73S, T78I, D115Q, S122G, V140L, K146T, |
| | | I148L, N168D, G187A, G195Y, I210C, S217N, S226C, K245Q, D256N, A258D, |
| | | I266N, K268G, E269G, D272S, E275N, Q282I, I286C, S301G, G319N, L324V, |
| | | E325S, D326S, Q330N, A356T, D359K |
| 408 | 27 | T36C, A40H, E42D, A43E, E45K, S46A, I47Q, S48Q, S51C, S69K, A71L, D73S, |
| | | T78I, M106Q, D115Q, S122G, D139K, V140L, F145I, K146T, I148L, I158V, V165L, |
| | | N168D, D179S, G187A, G195Y, I210C, S217N, S226C, K245Q, S250T, S252C, |
| | | S255T, D256N, A258T, I266N, K268G, E269G, D272S, E275N, I276F, Q282I, |
| | | I286C, S301G, S307T, I310V, D314N, V315F, G319N, L324V, E325A, D326S, |
| | | Q330N, D343N, S352T, A356T, D358T, D359K |
| 409 | 27 | T36C, A43E, S48Q, S51C, A71L, D73S, T78I, S122G, V140L, K146T, I148L, N168D, |
| | | G187A, G195Y, I210C, S217N, S226C, K245Q, D256N, A258D, I266N, K268G, |
| | | E269G, D272S, E275N, Q282I, I286C, S301G, G319N, L324V, E325S, D326S, |
| | | Q330N, A356T, D359K |
| 410 | 28 | D75E, K76T, K79E, V81L, L92Y, E99N, H109K, G113H, Y115F, E117K, S125A, |
| | | Y126K, V129Q, E133K, T143E, Y159R, D163P |
| 411 | 28 | D75E, K76T, K79E, V81L, L92M, H109K, G113H, S125A, Y126E, V129Q, D163P |
| 412 | 28 | D75E, K76T, V77T, K79E, V81L, L92M, E99N, H109K, T110Q, G113E, Y115F, |
| | | E117K, S125A, Y126K, V129Q, E133K, T143E, M147Q, Y159R, K161G, D163P |
| 413 | 28 | D75E, K76T, K79E, V81L, L92M, H109K, G113H, S125A, Y126K, V129Q, D163P |
| 414 | 28 | D75E, H109K, G113R, S125A, Y126K, V129Q, D163P |
| 416 | 28 | H109K, G113R, S125A, Y126E, V129Q, D163P |
| 417 | 28 | H109K, G113R, S125A, Y126K, V129Q, D163P |
| 418 | 28 | D74N, D75E, K76T, V77T, K79E, V81L, Y88F, L92M, E99N, H109K, T110Q, G113H, |
| | | Y115F, E117K, S125A, Y126K, V129Q, E133K, V137P, T143E, M147Q, Y159R, |
| | | K161G, D163P |
| 419 | 28 | D74N, D75E, K76T, V77T, K79E, V81L, Y88F, L92Y, E99N, R105Q, H109R, T110N, |
| | | G113H, Y115F, E117R, S125A, Y126K, V129Q, E133K, V137P, T143E, M147Q, |
| | | Y159R, K161G, D163P, A168S |
| 420 | 28 | D75E, K76T, K79E, V81L, L92Y, E99N, H109K, G113N, Y115F, E117K, S125A, |
| | | Y126E, V129Q, E133K, T143E, Y159R, D163P |
| 421 | 28 | D74N, D75E, K76T, V77T, K79E, V81L, Y88F, L92Y, E99N, H109K, T110Q, G113E, |
| | | Y115F, E117K, S125A, Y126K, V129Q, E133K, V137P, T143E, M147Q, Y159R, |
| | | K161G, D163P |
| 422 | 28 | D74N, D75E, K76T, V77T, K79E, V81L, Y88F, L92Y, E99N, R105Q, E106K, H109K, |
| | | T110Q, G113E, Y115F, E117K, S125A, Y126K, V129Q, E133K, V137P, T143E, |
| | | M147Q, Y159R, K161G, D163P, A168S |
| 423 | 28 | D75E, K76T, V77T, K79E, V81L, L92M, E99N, H109K, T110Q, G113N, Y115F, |
| | | E117K, S125A, Y126K, V129Q, E133K, T143E, M147Q, Y159R, K161G, D163P |
| 424 | 29 | T68G, E79A, T84N, P101S, L149V, E166Q, S173K, Q184N, E189S, K202A, N203S, |
| | | M206L, N207H, T210Q, L211M, F213Y, I216V, A218V, Y229Q, A230T, N232S |
| 425 | 29 | T68G, S73H, T76N, E79S, T84N, P101S, L149V, N161V, E166Q, S173R, Q184R, |
| | | E189S, T193N, Q195A, K202A, N203S, M206L, N207H, L211M, F213Y, I216V, |
| | | A218V, Y229I, A230T, N232S |
| 426 | 29 | T68G, S73H, E79S, T84N, P101S, L149V, N161V, E166Q, S173R, Q184R, E189S, |
| | | T193N, Q195A, H197F, K202A, N203S, M206L, N207H, L211M, F213Y, I216V, |
| | | A218V, Y229I, A230T, N232S |
| 427 | 29 | T68G, P101S, E166Q, Q184N, E189S, K202A, N203S, M206L, N207H, T210Q, |
| | | L211M, F213Y, I216V, A218V, Y229Q, A230S, N232S |
| 428 | 29 | T68G, E79A, P101S, E166Q, S173R, Q184N, E189S, K202A, N203S, M206L, |
| | | N207H, T210Q, L211M, F213Y, I216V, A218V, Y229Q, A230S, N232S |
| 429 | 29 | T68G, E79T, T84N, P101S, L149V, E166Q, S173K, Q184N, E189S, K202A, N203S, |
| | | M206L, N207H, T210Q, L211M, F213Y, I216V, A218V, Y229Q, A230T, N232S |
| 430 | 29 | Q184N, E189S, K202A, N203S, M206L, N207H, T210Q, L211M, F213Y, I216V, |
| | | A218V, Y229Q, A230S, N232S |
| 431 | 29 | T68G, S73H, T76N, E79S, T84N, P101S, L149V, N161V, E166Q, S173R, Q184R, |
| | | E189S, T193N, Q195A, H197F, K202A, N203S, M206L, N207H, L211M, F213Y, |
| | | I216V, A218V, Y229I, A230T, N232S |
| 432 | 29 | T68G, S73H, E79S, T84N, P101S, L149V, N161V, E166Q, S173R, Q184R, E189S, |
| | | K202A, N203S, M206L, N207H, T210Q, L211M, F213Y, I216V, A218V, Y229Q, |
| | | A230T, N232S |
| 433 | 29 | T68G, S73H, E79S, T84N, P101S, L149V, N161C, E166Q, S173R, Q184R, E189S, |
| | | T193N, Q195A, K202A, N203S, M206L, N207H, L211M, F213Y, I216V, A218V, |
| | | Y229I, A230T, N232S |
| 435 | 30 | R147D, A156Q, Q182T |
| 436 | 30 | I145T, R147D, A156Q, Q182T, A191R, N198G |
| 437 | 30 | S69T, A88N, D128N, I145T, R147D, A156Q, Q182T, A191R, N198G |
| 438 | 30 | I145T, R147D, A156Q, Q182T, N198G |
| 439 | 30 | S69T, A88N, F124Y, D128N, D135K, I145T, R147D, A156Q, D160E, Q182T, |
| | | A191R, L195M, N198G |
| 440 | 30 | S69T, A88N, D128N, I145T, R147D, A156Q, Q182N, A191R, N198G |
| 441 | 30 | R147D, A156Q, Q182T, N198G |
| 442 | 30 | S69E, A88N, F124Y, D128N, D135K, I145T, R147D, A156Q, D160E, Q182N, |
| | | A191R, L195M, N198G |
| 443 | 30 | I145T, R147D, A156Q, Q182N, N198G |
| 444 | 30 | R147D, A156Q, Q182N, N198G |
| 445 | 31 | A33V, K99Q, S100R, I102A, K103S, I107M, S130N, A140Q, W177C, M187I, K188A, |
| | | L207N, I210M, L227V, D252E, S284C, D289N, K306W, Y316A, K348S, K360N, |
| | | G364Q, N368T, S375T, L390M, G393T, N398T, T400S, D401G, H407W, A418V |
| 446 | 31 | K99Q, S100R, I102A, I107M, A140Q, T151E, L207N, L227V, D252R, S284C, |
| | | Y316A, K348S, K360N, L390M, N398T, T400S, D401G, H407W, A418V |
| 447 | 31 | A33V, K99Q, S100R, I102A, K103S, I107M, S130N, A140Q, W177C, M187I, K188A, |
| | | L207N, I210M, L227V, D252E, S284C, D289N, K348S, K360N, G364Q, N368T, |
| | | S375T, L390M, G393T, N398T, T400S, D401G, H407W, A418V |
| 448 | 31 | A33V, E65Q, Y92N, K99Q, S100R, K103A, I107K, V108L, N124T, K144A, K188A, |
| | | I199M, L207N, K219T, L227V, D252R, H280E, S284C, K306R, T310N, K348S, |
| | | D350G, L390M, G393T, N398T, T400S, D401G, H407W, A418V |
| 449 | 31 | A33V, R36A, R64M, E65Q, E86P, N90D, K99Q, S100R, K103A, H104R, I107K, |
| | | V108L, V120I, N124T, S130D, K144A, F152Y, L184A, M187I, K188A, A196C, |
| | | I199M, L207N, I210M, K219T, L227V, T234G, N237S, G248H, D252R, S278A, |
| | | H280E, S284C, D289N, K306R, T310N, K348S, D350G, V353I, N368T, L390M, |
| | | G393T, N396D, N398T, T400S, D401G, H403Y, H407W, A418V |
| 450 | 31 | A33V, E65Q, Y92N, K99Q, S100R, K103A, I107K, V108L, N124T, K144A, K188A, |
| | | I199M, L207N, K219T, L227V, D252R, H280E, S284C, K306R, T310N, K348S, |
| | | D350G, N368Q, L390M, G393T, N398T, T400S, D401G, H407W, A418V |
| 451 | 31 | H83G, K99Q, S100R, I102Q, K103A, A140Q, W177C, L207N, L227V, D252E, |
| | | S284C, D289N, K348S, K360N, S375T, L390M, G393T, N398T, T400S, D401G, |
| | | H407W, A418V |
| 452 | 31 | K99Q, S100R, I102A, K103A, A140Q, K154G, W177C, L207N, L227V, D252N, |
| | | S284C, D289N, Y316A, K348S, K360N, S375T, L390M, G393T, N398T, T400S, |
| | | D401G, H407W, A418V |
| 453 | 31 | K99Q, S100R, I102A, K103A, I107M, A140Q, W177C, L207N, L227V, D252N, |
| | | S284C, Y316A, K348T, K360N, S377T, L390M, G393T, N398T, T400S, D401G, |
| | | H407W, A418V |
| 454 | 31 | A33V, R36H, A39T, K41H, V43T, H83G, N90E, K99N, S100R, I102Q, K103A, |
| | | H104M, I107T, S117A, N124S, S130N, A140Q, K144Q, F152Y, K154Q, W177C, |
| | | I183F, L184R, M187L, K188A, A196C, L207N, I210M, P218A, K219T, L227V, |
| | | A229T, T234G, N237S, G248H, D252R, Y260H, S278A, S284C, D289N, T310N, |
| | | N317S, H320Q, Y343C, K348S, G349S, D350G, K360N, G364A, N368T, S375T, |
| | | S380T, I383M, L390M, Q391A, G393T, N396D, N398T, T400S, D401G, H403R, |
| | | H407W, A418I |
| 455 | 31 | A33V, E65Q, H83G, N90D, K99Q, S100R, K103A, I107K, V108L, N124T, K144A, |
| | | L184A, K188A, I199M, L207N, I210M, K219T, L227V, T234G, D252R, H280E, |
| | | S284C, K306R, T310N, K348S, D350G, L390M, G393T, N398T, T400S, D401G, |
| | | H407W, A418V |
| 456 | 31 | A33V, E65Q, S100R, K103E, N124T, K144A, K188A, I199M, L207N, L227S, D252R, |
| | | S284C, K306R, T310N, K348S, G393T, N398T, T400S, D401G, H407W, A418V |
| 457 | 31 | S100R, K103E, N124T, K144A, L207N, L227S, D252R, K306R, K348S, N398T, |
| | | T400S, D401G, H407W, A418V |
| 458 | 31 | A33V, R36S, A39T, K41H, V43T, N90E, K99Q, S100R, I102Q, K103A, H104L, |
| | | I107T, S117A, N124S, S130D, A140Q, K144Q, F152Y, K154Q, W177C, I183F, |
| | | L184R, M187L, K188A, N195S, A196C, L207N, I210M, P218A, K219T, L227V, |
| | | T234G, N237S, G248H, D252R, Y260H, S278A, S284C, D289N, T310N, Y316A, |
| | | K348S, G349S, D350G, K360N, G364Q, N366T, N368T, S375T, S377T, S380T, |
| | | I383M, L390M, G393T, N396D, N398T, T400S, D401G, H403R, H407W, A418I |
| 459 | 31 | A33V, V43T, K99Q, S100R, I102Q, K103A, H104R, I107T, N124S, S130N, A140Q, |
| | | K144A, D148S, F152Y, W177C, L184R, M187I, K188A, A196C, L207N, I210M, |
| | | K219T, L227V, T234G, Q235S, G248H, D252N, S284C, D289N, T310N, Y316A, |
| | | K348S, D350G, K360N, G364A, S375T, S377T, L390M, G393T, N398T, T400S, |
| | | D401G, H403R, H407N, A418I |
| 460 | 31 | K99Q, S100R, I102A, A140Q, T151E, W177C, L207N, L227V, D252R, S284C, |
| | | K348S, K360N, L390M, N398T, T400S, D401G, H407W, A418V |
| 461 | 31 | S100R, K103E, N124T, K144A, I199L, L207N, L227V, D252R, K306R, K348S, |
| | | N368Q, N398T, T400S, D401G, H407W, A418V |
| 462 | 31 | K99Q, S100R, I102A, K103A, L207N, L227V, D252E, S284C, K348T, K360N, |
| | | L390M, G393T, N398T, T400S, D401G, H407W, A418V |
| 463 | 31 | A33V, R64M, E65Q, N90D, K99Q, S100R, K103A, H104N, I107K, V108L, V120I, |
| | | N124T, K144A, F152Y, L184A, K188A, A196C, I199M, L207N, I210M, K219T, |
| | | L227V, T234G, G248H, D252R, S278A, H280E, S284C, K306R, T310N, K348S, |
| | | D350G, L390M, G393T, N398T, T400S, D401G, H407W, A418V |
| 464 | 31 | A33V, V43T, A61P, F68I, H83G, K99Q, S100R, I102Q, K103A, H104N, I107T, |
| | | N124S, S130N, F152Y, M187I, K188A, A196C, L207N, I210M, K219T, L227V, |
| | | T234G, G248H, D252N, S284C, D289N, Y316A, K348S, K360N, G364T, S375T, |
| | | S377T, L390M, G393T, N398T, T400S, D401G, H407Y, A418I |
| 465 | 31 | N398T, T400S, D401G, A418V |
| 466 | 31 | A33V, V43T, H83G, K99Q, S100R, I102Q, K103A, H104N, I107T, N124S, S130N, |
| | | A140Q, F152Y, W177C, M187I, K188A, A196C, L207N, I210M, K219T, L227V, |
| | | T234G, Q235S, G248H, D252N, S284C, D289N, Y316A, K348S, K360N, G364Q, |
| | | N368T, S375T, S377T, L390M, G393T, N398T, T400S, D401G, H407Y, A418I |
| 467 | 31 | A33V, E65Q, S100R, K103E, N124T, K144A, K188A, I199M, L207N, L227V, D252R, |
| | | S284C, K306R, T310N, K348S, G393T, N398T, T400S, D401G, H407W, A418V |
| 468 | 31 | A33V, R64M, E65Q, H83G, N90D, K99Q, S100R, K103A, H104N, I107K, V120I, |
| | | N124T, K144A, F152Y, L184A, K188A, A196C, I199M, L207N, I210M, K219T, |
| | | L227V, T234G, G248H, D252R, S278A, H280E, S284C, K306R, T310N, K348S, |
| | | D350G, L390M, G393T, N398T, T400S, D401G, H407W, A418V |
| 469 | 31 | A33V, V43T, K99Q, S100R, I102Q, K103A, H104R, I107T, N124S, S130N, A140Q, |
| | | K144A, D148S, F152Y, W177C, L184R, M187I, K188A, A196C, L207N, I210M, |
| | | K219T, L227V, T234G, G248H, D252N, S284C, D289N, T310N, Y316A, K348S, |
| | | D350G, K360N, G364A, S375T, S377T, L390M, G393T, N398T, T400S, D401G, |
| | | H403R, H407W, A418I |
| 470 | 31 | S100R, K103E, N124T, K144A, I199L, L207N, L227V, D252R, K306R, K348S, |
| | | N398T, T400S, D401G, H407W, A418V |
| 471 | 31 | A33V, R36A, R64M, E65Q, E86P, N90D, K99Q, S100R, K103A, H104R, I107K, |
| | | V108L, V120I, N124T, S130D, K144A, H145W, F152Y, L184A, M187I, K188A, |
| | | A196C, I199M, L207N, I210M, K219T, L227V, T234G, N237S, G248H, D252R, |
| | | S278A, H280E, S284C, D289N, K306R, T310N, K348S, D350G, V353I, N368T, |
| | | L390M, G393T, N396D, N398T, T400S, D401G, H403Y, H407W, A418V |
| 472 | 32 | F55Y, S62G, S79T, T83N, A88C, G96S, R97G, V100I, A104G, T115A, V130I, |
| | | S132N, W136Y, T145S, I146V, Q157Y, S191A, R200K, Y201H, S207T, M211Q, |
| | | Y223S, V228L |
| 473 | 32 | G96S, R97G, A104G, W136Y, T145S, I146V, Q157Y, S191A, Y201H, S207T, |
| | | Y223S, V228L |
| 474 | 32 | R97G, T145S, Q157Y, S191A, Y201H, S207T, Y223S, V228L |
| 475 | 32 | F55Y, S62G, S79T, T83N, A88C, R97G, V100I, A104G, T115A, V130I, S132N, |
| | | W136Y, T145S, I146V, Q157Y, S191A, R200K, Y201L, S207T, M211Q, Y223S, |
| | | V228L |
| 476 | 32 | R97G, W136Y, T145S, Q157Y, S191A, Y201H, S207T, Y223S, V228L |
| 477 | 32 | F55Y, G96S, R97G, A104G, T115A, W136Y, T145S, I146V, Q157Y, S191A, Y201H, |
| | | S207T, Y223S, V228L |
| 478 | 32 | S191A, Y201H, N204H, S207T, Y223S, V228L |
| 479 | 33 | V29T, D32H, F35A, K36A, A37R, T45S, N83S, S101K, G103N, L111T, V112L, |
| | | G128T, Q132R, G212A, N215A, H216K, V221L, A238S, A240Q, N249Q, G253S, |
| | | A269G, K279Q, L282C, V288C, S306E, S307T, A319P, T322N, I324C, A325V |
| 480 | 33 | V29T, K36A, A37Q, T45S, S101K, G103N, G128T, Q143G, V149C, R180E, N215A, |
| | | H216N, V225I, A240Q, G253S, G255N, K257S, A269G, L282C, V288C, S306E, |
| | | S307T, A319P, T322N, I324A |
| 481 | 33 | V29T, K34Q, F98L, S101Q, G103N, G128A, Q143G, R180E, V189A, N215A, |
| | | H216K, A240Q, N249Q, G253S, K257S, A269G, K279E, L282C, V288C, S307T, |
| | | A319P, T322N, I324A |
| 482 | 33 | V29T, G128A, Q143G, R180E, K257S, A269G, K279E, L282C, V288C, S307T, |
| | | A319P, T322N, I324A |
| 483 | 33 | G128K, Q143G, K257S, A269G, K279E, L282C, V288C, S307T, A319P, I324A |
| 484 | 33 | V29T, D32H, K36A, A37R, T45S, S101K, G103N, L111T, G128T, Q132R, N215A, |
| | | H216K, V221L, A240Q, G253S, A269G, K279Q, L282C, V288C, S307T, A319P, |
| | | T322N, I324C, A325V |
| 485 | 33 | V29T, D32H, F35M, K36A, A37R, T45S, N83S, S101K, G103N, L111T, G128A, |
| | | Q132R, N215A, H216K, V221L, A240Q, N249Q, G253S, A269G, K279Q, L282C, |
| | | V288C, V295I, S307T, A319P, T322N, I324C, A325V |
| 486 | 33 | D32N, K36A, L111T, G128T, Q132R, G253S, A269G, K279Q, L282C, V288C, |
| | | S307T, A319P, I324C |
| 487 | 33 | V29T, K34Q, F98L, S101Q, G103N, G128A, Q143G, R180E, V189A, N215A, |
| | | H216N, A240Q, N249Q, G253S, K257S, A269G, K279E, L282C, V288C, S307T, |
| | | A319P, T322N, I324A |
| 488 | 33 | V29T, K34Q, K36Q, F98L, S101Q, G103N, G128A, Q143G, I158A, R180E, V189A, |
| | | G212A, N215A, H216K, V221I, V225I, A238S, A240Q, N249Q, G253S, K257S, |
| | | A269G, V277I, L282C, V288C, S307T, A319P, T322N, I324A |
| 489 | 33 | V29T, F98L, S101Q, G103N, G128A, Q143G, R180E, V189A, N215A, H216N, |
| | | A240Q, G253S, K257S, A269G, K279E, L282C, V288C, S307T, A319P, T322N, |
| | | I324A |
| 490 | 33 | G128T, Q143G, G255N, K257S, A269G, L282C, V288C, S307T, A319P, I324A |
| 491 | 33 | V29T, K34Q, K36A, A37K, T45S, S101K, G103N, G128A, Q132S, Q143G, V149C, |
| | | R180E, G212A, N215A, H216K, K218D, V221L, V225I, A238S, A240Q, N249Q, |
| | | G253S, G255N, T256A, K257S, A269G, V277I, L282C, V288C, S306E, S307T, |
| | | A319P, T322N, I324A |
| 492 | 33 | V29T, D32H, F35M, K36A, T45S, N83S, S101K, G103N, L111T, G128T, Q132R, |
| | | N215A, H216K, V221L, A240Q, N249Q, G253S, A269G, K279Q, L282C, V288C, |
| | | S307T, A319P, T322N, I324C, A325V |
| 493 | 33 | S101T, G128T, Q143G, G253S, G255N, K257S, A269G, L282C, V288C, S307T, |
| | | A319P, I324A |
| 494 | 33 | V29T, K34Q, K36A, A37Q, T45S, S101K, G103N, G128T, Q143G, V149C, R180E, |
| | | N215A, H216N, V225I, A240Q, L251F, G253S, G255N, K257S, A269G, L282C, |
| | | V288C, S306E, S307T, A319P, T322N, I324A |
| 495 | 33 | V29T, K34E, F35M, K36A, A37K, T45S, S101K, G103N, G128K, Q132S, Q143G, |
| | | V149C, R180E, G212A, N215A, H216K, K218D, V221L, V225I, A238S, A240Q, |
| | | N249Q, G253S, G255N, T256A, K257S, A269G, V277I, L282C, V288C, S306E, |
| | | S307T, A319P, T322N, I324A, A325L |
| 496 | 33 | V29A, D32H, K36A, A37R, T45S, S101K, G103N, L111T, G128T, Q132R, N215A, |
| | | H216K, V221L, A240Q, G253S, A269G, K279Q, L282C, V288C, S307T, A319P, |
| | | T322N, I324C, A325V |
| 497 | 33 | D32H, K36A, L111T, G128T, Q132R, G253S, A269G, K279Q, L282C, V288C, |
| | | S307T, A319P |
| 498 | 33 | D32H, K36A, L111T, G128T, G253S, A269G, K279Q, L282C, V288C, S307T, A319P |
| 499 | 33 | G128A, Q143G, K257S, A269G, K279E, L282C, V288C, S307T, A319P, I324A |
| 500 | 33 | V29T, K34E, K36Q, F98L, S101Q, G103N, G128K, Q143G, I158A, R180E, V189A, |
| | | G212A, N215A, H216N, V221I, V225I, A238S, A240Q, N249Q, G253S, K257S, |
| | | A269G, V277I, L282C, V288C, S307T, A319P, T322N, I324A |
| 501 | 33 | G128A, Q143G, K257S, A269G, L282C, V288C, S307T, A319P |
| 502 | 33 | V29T, G103N, G128T, Q143G, R180E, V225I, G253S, G255N, K257S, A269G, |
| | | K279Q, L282C, V288C, S307T, A319P, T322N, I324A |
| 503 | 33 | S101T, G128T, Q143G, G255N, K257S, A269G, L282C, V288C, S307T, A319P, |
| | | I324A |
| 504 | 33 | V29T, S101T, G103N, G128T, Q143G, R180E, V225I, G253S, G255N, K257S, |
| | | A269G, K279Q, L282C, V288C, S307T, A319P, T322N, I324A |
| 505 | 33 | V29T, D32N, K36A, T45S, G103N, L111T, G128T, Q132R, N215A, A240Q, G253S, |
| | | A269G, K279Q, L282C, V288C, S307T, A319P, T322N, I324C |
| 506 | 33 | V29T, T45S, S101K, G103N, G128T, Q143G, R180E, V225I, A240Q, G253S, |
| | | G255N, K257S, A269G, K279E, L282C, V288C, S306E, S307T, A319P, T322N, |
| | | I324A |
| 507 | 33 | V29T, D32H, K34Q, F35M, K36A, A37R, T45S, N83S, S101K, G103N, L111T, |
| | | V112L, G128T, Q132R, G212A, N215A, H216K, V221L, A238S, A240Q, N249Q, |
| | | G253S, A269G, K279Q, L282C, V288C, S307T, A319P, T322N, I324C, A325V |
| 508 | 33 | V29T, F98L, S101Q, G103N, G128K, Q143G, R180E, V189A, N215A, H216N, |
| | | A240Q, G253S, K257S, A269G, K279E, L282C, V288C, S307T, A319P, T322N, |
| | | I324A |
| 509 | 33 | L282C, V288C, S307T, A319P |
| 510 | 34 | S53G, S67D, D83Q, T92Q, A97R, S104Q, N108E, E163Q, N180Q, H203N, L204M, |
| | | S226T |
| 511 | 34 | S53G, Q65N, S67C, N78T, D83Q, T92S, A97R, S104Q, N108E, E163Q, N180Q, |
| | | H203N, L204M, M207H, S226T |
| 512 | 34 | S53G, S67D, A97R, S104Q, N108E, E163Q, H203N, S226T |
| 513 | 34 | S53G, S67D, A97R, S104Q, N108E, E163Q, N180Q, H203N, S226T |
| 515 | 34 | S53G, Q65N, S67D, D83Q, T92S, A97R, S104Q, N108E, E163Q, N180Q, H203N, |
| | | L204M, S226T |
| 516 | 34 | A97R, E163Q |
| 517 | 34 | S53G, Q65N, S67C, N78T, D83Q, T92Q, A97R, S104Q, N108E, E163Q, N180Q, |
| | | H203N, L204M, M207H, S226T |
| 518 | 34 | S53G, A97R, E163Q |
| 519 | 34 | S53G, Q65N, S67C, D83Q, T92S, A97R, S104Q, N108E, E163Q, N180Q, H203N, |
| | | L204M, S226T |
| 520 | 35 | F29Y, N30D, N33K, Q37H, S39A, N55K, A62N, L74I, G113A, N116D, D118L, |
| | | K120T, E121D, N123S, R130K, G131A, D133R, T136Y, S137N, S138A, Q148A, |
| | | H157Y, K259V, A265S, P272T, L286V |
| 522 | 35 | F29Y, N30D, N33K, Q37H, S39A, N55K, A62N, L74I, G113A, N116D, D118L, |
| | | K120T, E121D, N123S, R130K, G131A, D133R, T136W, S137D, S138A, Q148A, |
| | | H157Y, K259V, A265S, P272T, L286V |
| 523 | 35 | F29Y, N33K, Q37H, L74I, G113A, T136W, S137E, H157Y, K259V |
| 524 | 35 | F29Y, N33K, Q37H, S39A, L74I, G113A, T136Y, S137N, H157Y, K259V |
| 525 | 35 | E23D, F29Y, N30D, N33K, Q37H, S39A, A52P, N55K, A62N, L74I, V82L, G113A, |
| | | N116D, D118L, K120T, E121D, N123S, R130K, G131A, D133R, T136Y, S137N, |
| | | S138A, A143R, Q148A, H157Y, K259V, I260C, A265S, Q271G, P272T, L286I |
| 526 | 35 | F29Y, N30D, N33K, Q37H, S39A, N55K, A62N, L74I, G113A, K120T, N123S, |
| | | R130K, D133R, T136Y, S137N, H157Y, K259V |
| 527 | 35 | F29Y, N33K, Q37H, S39A, N55K, L74I, G113A, N123S, D133R, T136Y, S137N, |
| | | H157Y, K259V |
| 528 | 35 | F29Y, N30D, N33K, Q37H, S39A, N55K, A62N, L74I, G113A, E121D, N123S, |
| | | R130K, D133K, T136Y, S137N, H157Y, K259V |
| 529 | 35 | F29Y, N33K, Q37H, L74I, G113A, T136W, S137N, H157Y, K259V |
| 530 | 36 | I66A, R98I, L102H, D113Q, E130D, A132G, L165M, E198N, G234A, A238K, S241C, |
| | | D244C, P248W, Y253C, D293C, S301C, P302S, K309M, V329K, F348Y, N351T, |
| | | E439M, N456M, S461N, I463V, P466A, A470E, T475S, S478G, S479T, S481A, |
| | | T482A, G483T, T486I, T491S, P492V |
| 531 | 36 | V43I, I66A, L90A, L102N, D113Q, E130D, A132G, L165M, S178R, E198N, T231A, |
| | | G234A, A238K, S241C, W242P, D244C, P248W, Y253C, D293C, I295A, S301C, |
| | | P302S, K309M, I325K, V329K, F348Y, N351T, T397S, G413E, A420Y, D433N, |
| | | T438A, E439M, N456M, S461N, I463V, P466A, A470E, T475S, S479T, S481A, |
| | | G483T, T486I, T491S, P492V |
| 532 | 36 | V43I, N46Q, Y58K, S61G, I66A, T73K, L90A, R98I, N99A, L102H, T106Q, V107T, |
| | | E109Q, D113Q, T122S, E130D, A132G, D148G, L165M, D167A, E174A, Y177N, |
| | | S178R, V182S, T183N, E198N, M214S, S215H, A220M, L226V, T231R, F232L, |
| | | G234A, V236L, A238K, S241C, W242P, D244C, P248W, Y253C, D293C, I295A, |
| | | S301C, P302S, N307T, K309M, V310A, V318I, L321I, I325K, E327S, V329K, F348Y, |
| | | S350A, N351T, D383Q, V390T, T397S, F400Y, D402N, T409W, G413D, Y414F, |
| | | I415M, S416A, T424S, S427N, Y432F, D433N, K434R, S435Y, T438A, E439M, |
| | | M458A, S461N, I463V, P466H, A470E, T475S, S479T, G483T, T486I, T491S, |
| | | P492V |
| 533 | 36 | I66A, L90A, L102N, D113Q, E130D, A132G, L165M, S178R, E198N, G234A, |
| | | A238K, S241C, D244C, P248W, Y253C, D293C, I295A, S301C, P302S, K309M, |
| | | I325K, V329K, F348Y, N351T, T397S, E439M, N456M, S461N, I463V, P466A, |
| | | A470E, T475S, S478G, S479T, S481A, T482A, G483T, T486I, T491S, P492V |
| 534 | 36 | I66A, L90A, L102N, D113Q, E130D, A132G, L165M, S178R, E198N, G234A, |
| | | A238K, S241C, D244C, P248W, Y253C, D293C, I295S, S301C, P302S, K309M, |
| | | I325K, V329K, F348Y, N351T, T397S, E439M, N456M, S461N, I463V, P466A, |
| | | A470E, T475S, S478G, S479T, S481A, T482A, G483T, T486I, T491S, P492V |
| 535 | 36 | V43I, I66A, L90A, L102N, D113E, E130D, A132G, L165M, S178R, E198N, T231A, |
| | | G234A, A238K, S241C, W242P, D244C, P248W, Y253C, D293C, I295S, S301C, |
| | | P302S, K309M, I325K, V329R, F348Y, N351T, T397S, G413E, A420Y, D433N, |
| | | T438A, E439M, N456M, S461N, I463V, P466A, A470E, T475S, S479T, S481A, |
| | | G483T, T486I, T491S, P492V |
| 536 | 36 | I66A, R98I, L102H, D113Q, E130D, A132G, L165M, E198A, G234A, A238K, S241C, |
| | | D244C, P248W, Y253C, D293C, S301C, P302S, K309M, V329K, F348Y, N351T, |
| | | E439M, N456M, S461N, I463V, P466A, A470E, T475S, S478G, S479T, S481A, |
| | | T482A, G483T, T486I, T491S, P492V |
| 537 | 36 | V43I, N46Q, Y58K, S61G, I66A, T73K, L90A, R98I, N99A, L102H, T106Q, V107T, |
| | | E109Q, D113E, T122S, E130D, A132G, D148G, L165M, D167A, E174A, Y177N, |
| | | S178R, V182S, T183N, E198N, M214S, S215H, A220M, L226V, T231R, F232L, |
| | | G234A, V236L, A238K, S241C, W242P, D244C, P248W, Y253C, D293C, I295A, |
| | | S301C, P302S, N307T, K309M, V310A, V318I, L321I, I325K, E327S, V329S, F348Y, |
| | | S350A, N351T, D383Q, V390T, T397S, F400Y, D402N, T409W, G413D, Y414F, |
| | | I415M, S416A, T424S, S427N, Y432F, D433N, K434R, S435Y, T438A, E439M, |
| | | M458A, S461N, I463V, P466H, A470E, T475S, S479T, G483T, T486I, T491S, |
| | | P492V |
| 538 | 36 | V43I, S44A, Y58K, S61G, I66A, L90A, L102N, D113Q, E130D, A132G, D148G, |
| | | L165M, S178R, E198N, M214S, L226V, T231A, G234A, A238K, S241C, W242P, |
| | | D244C, P248W, Y253C, D293C, I295A, S301C, P302S, K309M, I325K, E327Q, |
| | | V329S, F348Y, N351T, T397S, D402N, G413D, A420Y, D433N, T438A, E439M, |
| | | N456M, S461N, I463V, P466A, A470E, T475S, S479T, S481A, G483T, T486I, |
| | | T491S, P492V |
| 539 | 36 | V43I, S44A, Y58K, S61G, I66A, T73K, L90A, L102N, D113Q, E130D, A132G, |
| | | D148G, L165M, E174A, Y177N, S178R, E198N, M214S, S215H, L226V, T231A, |
| | | G234A, A238K, S241C, W242P, D244C, P248W, Y253C, D293C, I295T, S301C, |
| | | P302S, N307T, K309M, V310A, V318I, I325K, E327Q, V329S, F348Y, S350A, |
| | | N351T, V390T, T397S, F400Y, D402N, T409W, G413E, A420Y, D433N, K434R, |
| | | S435Y, T438A, E439M, N457H, M458A, S461N, I463V, P466H, A470E, T475S, |
| | | S479T, G483T, T486I, T491S, P492V |
| 540 | 36 | I66A, L102N, D113Q, E130D, A132G, L165M, E198N, G234A, A238K, S241C, |
| | | D244C, P248W, Y253C, D293C, I295A, S301C, P302S, K309M, V329K, F348Y, |
| | | N351T, E439M, N456M, S461N, I463V, P466A, A470E, T475S, S478G, S479T, |
| | | S481A, T482A, G483T, T486I, T491S, P492V |
| 541 | 36 | A38E, V43I, S44A, Y58K, S61G, I66A, T73K, L90A, L102N, D113Q, E130D, A132G, |
| | | D148G, L165M, E174A, Y177N, S178R, E198A, M214S, S215H, L226V, T231A, |
| | | G234A, A238K, S241C, W242P, D244C, P248W, Y253C, D293C, I295A, S301C, |
| | | P302S, N307T, K309M, V310A, V318I, I325K, E327Q, V329S, F348Y, S350A, |
| | | N351T, V390T, T397S, F400Y, D402N, T409W, G413D, D433N, K434R, S435Y, |
| | | T438A, E439M, N457H, M458A, S461N, I463V, P466H, A470E, T475S, S479T, |
| | | G483T, T486I, T491S, P492V |
| 542 | 36 | V43I, S44A, Y58K, S61G, I66A, L90A, L102N, D113E, E130D, A132G, D148G, |
| | | L165M, S178R, E198N, M214S, L226V, T231A, G234A, A238K, S241C, W242P, |
| | | D244C, P248W, Y253C, D293C, I295A, S301C, P302S, K309M, I325K, E327Q, |
| | | V329K, F348Y, N351T, T397S, D402N, G413D, A420Y, D433N, T438A, E439M, |
| | | N456M, S461N, I463V, P466A, A470E, T475S, S479T, S481A, G483T, T486I, |
| | | T491S, P492V |
| 543 | 36 | S241C, D244C, Y253C, D293C, S301C, S461N, G462S, I463V, P466A, A470E, |
| | | N474S, T475S, S478G, S479T, S481A, T482A, G483T, T486I, T491S, P492V |
| 544 | 37 | A14D, E28D, S33R, H45K, C84K, D99P, H117Y, E118S, D128E, R143I, K145Y, |
| | | V147A, D151K, V185I, D186Q, I190K, A207E, S208N, S217T, R219K, R224D, |
| | | N228K, Q232N, E236Q, T253Q, D276E, D280E, Q285K, S301Y, N313D, E315T, |
| | | I316E, T337K, A341K, L342F, A371T, F379Y, V395K, N398D, G421Q, R428E, |
| | | R429N, E431G, F433Y, D439K, I447L, E474D, E483D, G489E, A490K, Q493K, |
| | | E520Q, Y521K, A542E, K550Q, A551Q, T559N, N560Y, S585K, E617H, V624I, |
| | | T626W, E637R, E638Q, K641S, T642K, K650E, V651Q, M652L, Q654R, S679N, |
| | | K688I, S689D, T707S, D711I, G740A, L751I, V754A, N762W, Q798K, T808E, |
| | | E813D, V828T, D829G, Q830E, V842I, A844H, V846I, R863S, E877K, Q889M, |
| | | L915E, S941N, V967S, Q975N, H989F, T999N, K1080Q, S1146C, S1163L, L1168V, |
| | | G1169P, S1177Q, V1194I, K1195R, D1197E, K1259H, S1281V, S1296E, D1348N, |
| | | A1349G |
| 545 | 37 | A14D, E28D, S33R, H45K, C84K, N92D, D99P, H117Y, E118S, D128E, R143I, |
| | | K145Y, V147A, D151Q, L162C, M165L, V185I, D186Q, I190K, Q194D, A207E, |
| | | S208N, S217T, R219K, R224D, N228K, Q232N, E236Q, T253Q, A263E, D276E, |
| | | D280E, Q285K, S301Y, N313D, E315T, I316Q, Q334K, T337E, A341K, L342F, |
| | | A371T, F379Y, V395K, N398D, I418V, G421Q, R428E, R429N, E431G, F433Y, |
| | | D439E, I447L, S473T, E474D, E483D, G489E, A490K, Q493N, S494A, F502K, |
| | | E520Q, Y521K, V534I, A542E, A551Q, L555Y, T559K, N560Y, R561K, S585K, |
| | | E617H, V624I, T626W, E637R, E638Q, K641S, T642K, K650E, V651Q, M652L, |
| | | Q654R, K656E, K688I, S689D, A693N, T707S, D711I, G740A, L751I, V754A, |
| | | N762W, Q798K, T808E, E813D, K814R, V828T, D829G, V842I, A844H, V846I, |
| | | R863S, E877K, Q889M, F901Y, L915E, S941N, V967S, Q975D, H989F, T999N, |
| | | K1080S, F1084L, V1087I, S1146C, S1163L, L1168V, G1169P, S1177Q, E1193N, |
| | | V1194I, K1195R, D1197E, S1234Y, K1259H, Q1260K, L1261K, S1281V, V1284H, |
| | | S1296E, H1315N, D1348N, A1349G |
| 546 | 37 | A14D, E28D, S33A, H45K, S46Y, C84K, D99P, H117Y, E118S, D128E, R143I, |
| | | K145Y, V147A, D151Q, L162C, M165L, V185I, D186Q, I190N, A207T, S208N, |
| | | S217T, R219K, R224D, N228K, Q232N, E236Q, T253Q, A263E, D276E, D280E, |
| | | Q285K, L295Q, S301Y, N313D, E315T, I316E, T337K, A341K, L342F, A371T, |
| | | F379Y, P382K, V395K, N398D, G421Q, R428E, R429N, E431G, F433Y, D439K, |
| | | I447L, S473T, E474D, E483D, G489E, A490K, Q493N, S494A, E520Q, Y521K, |
| | | V534I, A542E, K550Q, A551Q, L555Y, T559K, N560Y, S585K, E617H, V624I, |
| | | T626W, E637R, E638Q, K641S, T642K, K650E, V651Q, M652L, Q654R, K656E, |
| | | S679N, K688I, S689D, T707S, D711I, G740A, L751I, V754A, N762W, T808E, |
| | | E813D, V828T, D829G, Q830E, V842I, A844H, V846I, R863S, E877P, Q889M, |
| | | F901Y, L915E, S941N, V967S, Q975D, H989F, T999N, K1080Q, S1146C, S1163L, |
| | | L1168V, G1169P, S1177Q, V1194I, K1195R, D1197E, K1259H, Q1260K, S1281V, |
| | | S1296E, H1315N, D1348N, A1349G |
| 547 | 37 | A14D, E28D, S33R, R44K, H45K, C84K, S91A, N92E, D99P, H117Y, E118S, |
| | | D128E, R143I, K145Y, V147A, D151E, L162C, M165L, V185I, D186Q, I190K, |
| | | Q194D, N206S, A207E, S208N, S217T, R219K, R224D, N228K, Q232N, E236Q, |
| | | S249A, T253Q, S258T, A263E, D276E, D277E, D280E, Q289E, S301Y, N313D, |
| | | E315T, I316N, S324A, Q334K, T337K, A341Q, L342F, A371T, F379Y, P382K, |
| | | V395K, N398D, I418V, G421Q, R428E, R429N, E431G, D432K, F433Y, D439E, |
| | | I447L, T470Q, S473T, E474D, E483D, G489E, A490K, Q493K, S494A, F502K, |
| | | K510D, E520Q, Y521K, V534I, A542E, K550Q, A551E, D554E, L555H, T559K, |
| | | N560Y, R561K, C578G, S585K, D609E, E617H, V624I, T626W, E637R, E638Q, |
| | | R639Q, K641S, T642K, A644S, H645D, D649E, K650E, V651Q, M652L, Q654R, |
| | | K656E, R668K, S679N, K688I, S689D, A693N, T707S, D711E, G740A, V747I, |
| | | L751I, V754A, N762W, M785Y, Q798D, T808E, E813D, K814R, V828T, D829G, |
| | | Q830E, V842I, A844H, V846I, K859R, R863S, D865K, E877K, Q889M, N892D, |
| | | F901Y, L915E, S941E, D951G, L953R, V967S, Q975N, H989F, T999N, K1080Q, |
| | | S1092N, T1142N, S1146C, S1163L, L1168V, G1169P, S1177K, E1193N, V1194I, |
| | | K1195R, D1197E, S1234Q, K1259N, Q1260K, L1261K, S1281V, V1284H, D1292E, |
| | | S1296Q, H1315N, D1348N, A1349G |
| 548 | 37 | A14D, E28D, S33R, H45K, C84K, N92D, D99P, H117Y, E118S, D128E, R143I, |
| | | K145Y, V147A, D151Q, L162C, M165L, V185I, D186Q, I190K, Q194D, A207E, |
| | | S208N, S217T, R219K, R224D, N228K, Q232N, E236Q, T253Q, A263E, D276E, |
| | | D280E, Q285K, L295Q, S301Y, N313D, E315T, I316Q, Q334K, T337K, A341K, |
| | | L342F, A371T, F379Y, P382K, V395K, N398D, I418V, G421Q, R428E, R429N, |
| | | E431G, F433Y, D439E, I447L, S473T, E474D, E483D, G489E, A490K, Q493K, |
| | | S494A, F502K, E520Q, Y521K, V534I, A542E, K550Q, A551Q, L555Y, T559K, |
| | | N560Y, R561K, S585K, E617H, V624I, T626W, E637R, E638Q, K641S, T642K, |
| | | K650E, V651Q, M652L, Q654R, K656E, S679N, K688I, S689D, A693N, T707S, |
| | | D711I, G740A, L751I, V754A, N762W, Q798K, T808E, E813D, K814R, V828T, |
| | | D829G, Q830E, V842I, A844H, V846I, R863S, E877K, Q889M, F901Y, L915E, |
| | | S941N, V967S, Q975D, H989F, T999N, K1080S, F1084L, V1087I, S1146C, S1163L, |
| | | L1168V, G1169P, S1177Q, E1193N, V1194I, K1195R, D1197E, S1234Y, K1259H, |
| | | Q1260K, L1261K, S1281V, V1284H, S1296E, H1315N, D1348N, A1349G |
| 549 | 37 | A14D, E28D, S33R, H45K, C84K, D99P, H117Y, E118S, D128E, R143I, K145Y, |
| | | V147A, D151Q, L162V, M165L, V185I, D186Q, I190N, A207E, S208N, S217T, |
| | | R219K, R224D, N228K, Q232N, E236Q, N244Q, T253Q, N259T, A263E, D276E, |
| | | D280E, Q285K, S301Y, N313D, E315T, I316E, T337K, L342F, A371T, F379Y, |
| | | V395E, N398D, R399K, G421Q, R428E, R429N, E431G, F433Y, D439K, I447L, |
| | | S473T, E474D, E483D, G489E, A490K, Q493K, S494A, E520Q, Y521K, V534I, |
| | | A542E, K550Q, A551E, L555Y, T559K, N560Y, S585K, E617H, V624I, T626W, |
| | | E637R, E638Q, K641S, T642K, K650E, V651Q, M652L, Q654R, K656E, S679N, |
| | | K688I, S689D, T707S, D711I, G740A, L751I, V754A, N762W, Q798K, T808E, |
| | | E813D, V828T, D829G, Q830E, V842I, A844H, V846I, R863S, E877K, Q889M, |
| | | F901Y, L915E, S941N, V967S, Q975D, H989F, T999N, K1080Q, S1146C, S1163L, |
| | | L1168V, G1169P, S1177Q, V1194I, K1195R, D1197E, K1259H, Q1260K, S1281V, |
| | | S1296E, H1315N, D1348N, A1349G |
| 550 | 37 | K8R, A14D, E28D, S33R, H45K, C84K, S91A, N92D, D99P, S100N, K116R, H117G, |
| | | E118S, D128E, R143I, K145Y, V147A, D151Q, L162I, M165L, V185I, D186Q, I190K, |
| | | Q194D, A207E, S208N, S217T, R219K, R224D, N228K, Q232N, E236Q, T253Q, |
| | | A263E, D276E, D280E, Q285K, S301Y, N313D, E315T, I316E, Q334K, T337K, |
| | | A341Q, L342F, A371T, F379Y, P382K, V395E, N398D, R399K, I418V, G421Q, |
| | | R428E, R429N, E431G, D432K, F433Y, D439E, I447L, S473T, E474D, E483D, |
| | | G489E, A490K, Q493K, S494A, F502K, E520Q, Y521K, V534I, A542E, K550Q, |
| | | A551Q, T559K, N560Y, R561K, S585K, E617H, V624I, T626W, E637R, E638Q, |
| | | K641S, T642K, K650E, V651Q, M652L, Q654R, K656E, R668K, K688I, S689D, |
| | | A693I, T707S, D711E, G740A, L751I, V754A, H758Q, N762S, Q798K, T808E, |
| | | E813D, K814R, V828T, D829G, Q830E, V842I, A844H, V846I, R863S, D865K, |
| | | E877K, Q889M, F901Y, L915E, S941E, L953R, Q975D, H989F, T999N, S1146C, |
| | | S1163L, L1168V, G1169P, S1177K, E1193N, V1194I, K1195R, D1197E, S1234Q, |
| | | N1238K, K1259H, Q1260K, L1261K, S1281V, V1284H, S1296E, H1315N, D1348N, |
| | | A1349G |
| 551 | 37 | K8R, A14D, E28D, S33R, R44K, H45K, C84K, S91A, N92E, D99P, S100N, K116R, |
| | | H117G, E118S, D128E, R143I, K145Y, V147A, D151E, L162C, M165L, V185I, |
| | | D186Q, I190K, Q194D, N206S, A207E, S208N, S217T, R219K, R224D, N228K, |
| | | Q232N, E236Q, S249A, T253Q, S258T, A263E, D276E, D280E, Q285K, S301Y, |
| | | N313D, E315T, I316N, S324A, Q334K, T337K, A341Q, L342F, Q346Y, A371T, |
| | | F379Y, P382K, V395K, N398D, I418V, G421Q, R428E, R429N, E431G, D432K, |
| | | F433Y, D439E, I447L, T470Q, S473T, E474D, E483D, G489E, A490K, Q493K, |
| | | S494A, F502K, E520Q, Y521K, V534I, A542E, K550Q, A551Q, L555Y, T559K, |
| | | N560Y, R561K, K574R, S585K, E617H, V624I, T626W, E637R, E638Q, R639Q, |
| | | K641S, T642K, H645D, D649E, K650E, V651Q, M652L, Q654R, K656E, R668K, |
| | | S679N, K688I, S689D, A693N, T707S, D711I, G740A, V747I, L751I, V754A, |
| | | N762W, M785Y, Q798D, T808E, E813D, K814R, V828T, D829G, V842I, A844H, |
| | | V846I, K859R, R863S, D865K, E877K, Q889M, N892D, F901Y, L915E, S941E, |
| | | D951G, Q975D, H989F, T999N, K1080Q, S1092N, T1142N, S1146C, S1163P, |
| | | L1168V, G1169P, S1177K, E1193N, V1194I, K1195R, D1197E, S1234Q, N1238K, |
| | | K1259N, Q1260K, L1261K, S1281V, V1284H, S1296E, H1315N, D1348N, A1349G |
| 552 | 37 | K8R, A14D, E28N, S33R, R44K, H45K, C84K, S91A, N92D, D99P, S100N, H117Y, |
| | | E118S, D128E, R143I, K145Y, V147A, D151E, L162V, M165L, V185I, D186Q, |
| | | I190N, Q194K, N206S, A207E, S208N, S217T, R219K, R224D, N228K, Q232N, |
| | | E236Q, S249A, T253Q, S258T, A263E, D276E, D280E, Q285K, S301Y, N313D, |
| | | E315T, I316N, S324A, Q334K, T337K, A341Q, L342F, Q346Y, A371T, F379Y, |
| | | P382K, V395K, N398D, I418V, G421Q, R428E, R429N, E431G, D432K, F433Y, |
| | | D439E, I447L, S473T, E474D, E483D, G489E, A490K, Q493K, S494A, F502K, |
| | | E520Q, Y521K, V534I, A542E, K550Q, A551Q, D554E, L555Y, T559K, N560Y, |
| | | R561K, S585K, E617H, V624I, T626W, E637R, E638Q, R639Q, K641S, T642K, |
| | | H645D, D649E, K650E, V651Q, M652L, Q654R, K656A, R668K, K688I, S689D, |
| | | A693N, T707S, D711I, G740A, V747I, L751I, V754A, H758Q, N762W, M785Y, |
| | | Q798D, T808E, E813D, K814R, V828T, D829G, Q830E, V842I, A844H, V846I, |
| | | K859R, R863S, D865K, E877K, Q889M, N892D, F901Y, L915E, S941E, D951G, |
| | | L953R, V967S, Q975D, H989F, T999N, K1080Q, V1087I, S1092N, T1142N, |
| | | S1146C, S1163P, L1168V, G1169P, S1177K, E1193N, V1194I, K1195R, D1197E, |
| | | S1234Q, K1259H, Q1260K, L1261K, S1281V, V1284H, S1296E, H1315N, D1348N, |
| | | A1349G |
| 553 | 37 | A14D, D27E, E28N, S33R, R44K, H45K, C84K, S91A, N92E, D99P, S100N, K116R, |
| | | H117G, E118S, D128E, A131E, K135Q, R143I, K145Y, V147A, D151E, L162V, |
| | | M165L, V185I, D186Q, I190N, Q198N, N206S, A207T, S208N, L216F, S217T, |
| | | R219K, R224D, N228K, Q232N, E236Q, S249A, L252N, T253Q, S258K, A263E, |
| | | D276E, D277E, D280E, Q285K, Q289E, L295N, S301Y, N313D, E315T, I316N, |
| | | S324A, E331D, Q334K, T337K, A341Q, L342F, Q346Y, A371T, S372T, E375D, |
| | | F379Y, E392Y, V395K, N398D, I418V, G421Q, R428E, R429N, E431G, D432K, |
| | | F433Y, D439E, I447L, S473T, E474D, E483D, G489E, A490K, Q493K, S494A, |
| | | F502K, K510D, E520Q, Y521K, V534I, A542E, K550Q, A551Q, L555H, T559K, |
| | | N560Y, R561K, C578G, F579Y, S585K, D609E, N613D, E615K, E617H, V624I, |
| | | T626W, E637R, E638Q, R639Q, K641S, T642K, A644S, H645D, D649E, K650E, |
| | | V651Q, M652L, Q654R, K656E, R668K, S679N, K688I, S689D, A693N, T707S, |
| | | D711I, Q713E, G740A, V747I, L751I, V754A, N762W, M785Y, Q798N, E806D, |
| | | T808Q, E813D, K814R, V828T, D829G, Q830E, V842I, A844H, V846I, K859R, |
| | | R863S, S864R, D865K, E877K, Q889M, N892E, F901Y, S913T, L915E, S941E, |
| | | D951G, L953R, K965N, V967S, Q975N, H989F, T999N, K1080S, F1084L, V1087I, |
| | | S1092N, N1119D, T1142N, S1146C, S1163L, L1168V, G1169P, S1177K, E1193N, |
| | | V1194I, K1195R, D1197E, S1234Q, V1237I, L1242N, K1259N, Q1260K, L1261K, |
| | | F1262Y, Q1265E, E1275N, S1281V, R1283K, V1284H, L1286G, D1288K, D1292E, |
| | | L1295E, S1296K, H1315N, D1348N, A1349G |
| 554 | 37 | A14D, E28D, S33R, H45K, C84K, S91A, N92E, D99P, S100N, K116R, H117G, |
| | | E118S, D128E, R143I, K145Y, V147A, D151Q, L162C, M165L, V185I, D186Q, |
| | | I190K, Q194D, A207E, S208N, S217T, R219K, R224D, N228K, Q232N, E236Q, |
| | | N244Q, T253Q, N259T, A263E, D276E, D280E, Q285K, S301Y, N313D, E315T, |
| | | I316Q, Q334K, T337K, A341E, L342F, A371T, F379Y, P382K, V395E, N398D, |
| | | R399K, I418V, G421Q, R428E, R429N, E431G, D432K, F433Y, D439E, I447L, |
| | | S473T, E474D, G489E, A490K, Q493K, S494A, F502K, E520Q, Y521K, A542E, |
| | | K550Q, A551Q, L555Y, T559K, N560Y, R561K, S585K, E617H, V624I, T626W, |
| | | E637R, E638Q, K641S, T642K, K650E, V651Q, M652L, Q654R, K656E, R668K, |
| | | S679N, K688I, S689D, A693I, T707S, D711E, G740A, L751I, V754A, H758Q, |
| | | N762S, Q798K, T808E, E813D, K814R, V828T, D829G, V842I, A844H, V846I, |
| | | R863S, D865K, E877K, Q889M, F901Y, L915E, S941E, L953R, V967S, Q975N, |
| | | H989F, T999N, S1146C, S1163L, L1168V, G1169P, S1177K, E1193N, V1194I, |
| | | K1195R, D1197E, S1234Q, K1259N, Q1260K, L1261K, S1281V, V1284H, S1296E, |
| | | H1315N, D1348N, A1349G |
| 555 | 37 | K8R, A14D, E28D, S33R, H45K, C84K, D99P, H117Y, E118S, D128E, R143I, |
| | | K145Y, V147A, D151K, L162I, V185I, D186Q, I190N, A207T, S208N, S217T, |
| | | R219K, R224D, N228K, Q232N, E236Q, T253Q, D276E, D280E, Q285K, S301Y, |
| | | N313D, E315T, I316Q, T337K, A341K, L342F, A371T, F379Y, V395K, N398D, |
| | | G421Q, R428E, R429N, E431G, F433Y, D439K, I447L, S473T, E474D, E483D, |
| | | G489E, A490K, Q493K, E520Q, Y521K, V534I, A542E, K550Q, A551Q, L555Y, |
| | | T559N, N560Y, S585K, E617H, V624I, T626W, E637R, E638Q, K641S, T642K, |
| | | K650E, V651Q, M652L, Q654R, S679N, K688I, S689D, T707S, D711I, G740A, |
| | | L751I, V754A, N762W, Q798K, T808E, E813D, V828T, D829G, V842I, A844H, |
| | | V846I, R863S, E877K, Q889M, F901Y, L915E, S941N, V967S, Q975D, H989F, |
| | | T999N, K1080Q, S1146C, S1163L, L1168V, G1169P, S1177Q, V1194I, K1195R, |
| | | D1197E, K1259H, S1281V, S1296E, D1348N, A1349G |
| 556 | 37 | A14D, E28N, S33R, R44K, H45K, C84K, S91A, N92E, D99P, H117Y, E118S, |
| | | D128E, K135Q, R143I, K145Y, V147A, D151E, L162V, M165L, V185I, D186Q, |
| | | I190K, Q194D, Q198N, N206S, A207E, S208N, L216F, S217T, R219K, R224D, |
| | | N228K, Q232N, E236Q, S249A, L252N, T253Q, S258K, A263E, D276E, D277E, |
| | | D280E, Q289E, L295N, S301Y, D308G, N313D, E315T, I316N, S324A, E331D, |
| | | Q334K, T337K, A341Q, L342F, Q346Y, A371T, S372T, E375D, F379Y, P382K, |
| | | E392Y, V395K, N398D, I418V, G421Q, R428E, R429N, E431G, D432K, F433Y, |
| | | D439E, I447L, E474D, E483D, G489E, A490K, Q493K, S494A, F502K, K510D, |
| | | E520Q, Y521K, V534I, A542E, K550Q, A551Q, L555H, T559K, N560Y, R561K, |
| | | C578G, F579Y, S585K, D609E, N613D, E615K, E617H, V624I, T626W, E637R, |
| | | E638Q, R639Q, K641S, T642K, A644S, H645D, D649E, K650E, V651Q, M652L, |
| | | Q654R, K656A, R668K, S679N, K688I, S689D, A693N, T707S, D711I, Q713E, |
| | | G740A, V747I, L751I, V754A, H758Q, N762W, M785Y, Q798N, E806D, T808Q, |
| | | E813D, K814R, V828T, D829G, V842I, A844H, V846I, K859R, R863S, S864R, |
| | | D865K, E877K, Q889M, N892E, F901Y, S913T, L915E, S941E, D951G, L953R, |
| | | K965N, V967S, Q975D, H989F, T999N, F1084L, V1087I, S1092N, N1119D, |
| | | T1142N, S1146C, S1163L, L1168V, G1169P, S1177K, E1193N, V1194I, K1195Q, |
| | | D1197N, S1234Q, V1237I, N1238K, K1259H, Q1260K, L1261K, F1262Y, Q1265E, |
| | | E1275N, S1281V, R1283K, V1284H, L1286G, D1292K, S1296Q, H1315N, D1348N, |
| | | A1349G |
| 557 | 37 | A14D, E28D, S33R, R44K, H45K, C84K, S91A, N92E, D99P, S100N, H117Y, |
| | | E118S, D128E, R143I, K145Y, V147A, D151E, L162V, M165L, V185I, D186Q, |
| | | I190K, Q194D, N206S, A207E, S208N, S217T, R219K, R224D, N228E, Q232N, |
| | | E236Q, S249A, T253Q, S258T, A263E, D276E, D277E, D280E, Q285K, Q289E, |
| | | S301Y, D308G, N313D, E315T, I316N, S324A, Q334K, T337K, A341Q, L342F, |
| | | Q346Y, A371T, F379Y, V395K, N398D, I418V, G421Q, R428E, R429N, E431G, |
| | | D432K, F433Y, D439E, I447L, T470Q, S473T, E474D, E483D, G489E, A490K, |
| | | Q493K, S494A, F502K, K510D, E520Q, Y521K, V534I, A542E, K550Q, A551Q, |
| | | D554E, L555H, T559K, N560Y, R561K, C578G, S585K, D609E, E617H, V624I, |
| | | T626W, E637R, E638Q, R639Q, K641S, T642K, A644S, H645D, D649E, K650E, |
| | | V651Q, M652L, Q654R, K656E, R668K, S679N, K688I, S689D, A693N, T707S, |
| | | D711I, G740A, V747I, L751I, V754A, H758Q, N762W, M785Y, Q798D, T808E, |
| | | E813D, K814R, V828T, D829G, Q830E, V842I, A844H, V846I, K859R, R863S, |
| | | D865K, E877K, Q889M, N892E, F901Y, L915E, S941E, D951G, V967S, Q975N, |
| | | H989F, T999N, K1080Q, V1087I, S1092N, T1142N, S1146C, S1163L, L1168V, |
| | | G1169P, S1177K, E1193N, V1194I, K1195R, D1197E, S1234Q, L1242N, K1259N, |
| | | Q1260K, L1261K, S1281V, V1284H, D1292E, S1296Q, H1315N, D1348N, A1349G |
| 558 | 38 | A6G, T309I, F398L, V404L, T405I |
| 559 | 38 | A6G, D117E, K155L, A157S, T303K, T309I, I366E, S384K, F398L, V404I, T405V, |
| | | N477H |
| 560 | 38 | A6G, T309I, I366E, F398L, V404I, T405V, N477H |
| 561 | 38 | A6G, T309I, F398L, V404I, T405I, N477H |
| 562 | 38 | A6G, D117E, T309I, I366E, S384K, F398L, V404I, T405V, N477H |
| 563 | 38 | A6G, D117E, K155L, T309I, I366E, S384K, F398L, V404I, T405V, N477H |
| 564 | 38 | A2K, A6G, D117E, K155L, A157S, S228E, K288D, A293K, T303K, T309I, I366D, |
| | | K367E, S384K, F398L, L403K, V404I, T405V, L409E, E410Q, N477H |
| 565 | 38 | A6G, D117E, K155L, T309I, I366E, S384K, F398L, V404I, T405I, N477H |

Any patent, patent application publication, or scientific publication, cited herein, is incorporated by reference herein in its entirety.

The following examples are presented in order to more fully illustrate the preferred embodiments of the invention. They should in no way be construed, however, as limiting the broad scope of the invention.

1-266. (canceled)

267. A Cas9 variant of a wild-type Cas9 from:
1) Streptococcus pyogenes of SEQ ID NO: 37 comprising mutations:
a) A14D, E28D, S33R, H45K, C84K, D99P, H117Y, E118S, D128E, R143I, K145Y, V147A, D151K, V185I, D186Q, I190K, A207E, S208N, S217T, R219K, R224D, N228K, Q232N, E236Q, T253Q, D276E, D280E, Q285K, S301Y, N313D, E315T, I316E, T337K, A341K, L342F, A371T, F379Y, V395K, N398D, G421Q, R428E, R429N, E431G, F433Y, D439K, I447L, E474D, E483D, G489E, A490K, Q493K, E520Q, Y521K, A542E, K550Q, A551Q, T559N, N560Y, S585K, E617H, V624I, T626W, E637R, E638Q, K641S, T642K, K650E, V651Q, M652L, Q654R, S679N, K688I, S689D, T707S, D711I, G740A, L751I, V754A, N762W, Q798K, T808E, E813D, V828T, D829G, Q830E, V842I, A844H, V846I, R863S, E877K, Q889M, L915E, S941N, V967S, Q975N, H989F, T999N, K1080Q, S1146C, S1163L, L1168V, G1169P, S1177Q, V1194I, K1195R, D1197E, K1259H, S1281V, S1296E, D1348N, and A1349G;
b) A14D, E28D, S33R, H45K, C84K, N92D, D99P, H117Y, E118S, D128E, R143I, K145Y, V147A, D151Q, L162C, M165L, V185I, D186Q, I190K, Q194D, A207E, S208N, S217T, R219K, R224D, N228K, Q232N, E236Q, T253Q, A263E, D276E, D280E, Q285K, S301Y, N313D, E315T, I316Q, Q334K, T337E, A341K, L342F, A371T, F379Y, V395K, N398D, I418V, G421Q, R428E, R429N, E431G, F433Y, D439E, I447L, S473T, E474D, E483D, G489E, A490K, Q493N, S494A, F502K, E520Q, Y521K, V534I, A542E, A551Q, L555Y, T559K, N560Y, R561K, S585K, E617H, V624I, T626W, E637R, E638Q, K641S, T642K, K650E, V651Q, M652L, Q654R, K656E, K688I, S689D, A693N, T707S, D711I, G740A, L751I, V754A, N762W, Q798K, T808E, E813D, K814R, V828T, D829G, V842I, A844H, V846I, R863S, E877K, Q889M, F901Y, L915E, S941N, V967S, Q975D, H989F, T999N, K1080S, F1084L, V1087I, S1146C, S1163L, L1168V, G1169P, S1177Q, E1193N, V1194I, K1195R, D1197E, S1234Y, K1259H, Q1260K, L1261K, S1281V, V1284H, S1296E, H1315N, D1348N, and A1349G;
c) A14D, E28D, S33A, H45K, S46Y, C84K, D99P, H117Y, E118S, D128E, R143I, K145Y, V147A, D151Q, L162C, M165L, V185I, D186Q, I190N, A207T, S208N, S217T, R219K, R224D, N228K, Q232N, E236Q, T253Q, A263E, D276E, D280E, Q285K, L295Q, S301Y, N313D, E315T, I316E, T337K, A341K, L342F, A371T, F379Y, P382K, V395K, N398D, G421Q, R428E, R429N, E431G, F433Y, D439K, I447L, S473T, E474D, E483D, G489E, A490K, Q493N, S494A, E520Q, Y521K, V534I, A542E, K550Q, A551Q, L555Y, T559K, N560Y, S585K, E617H, V624I, T626W, E637R, E638Q, K641S, T642K, K650E, V651Q, M652L, Q654R, K656E, S679N, K688I, S689D, T707S, D711I, G740A, L751I, V754A, N762W, T808E, E813D, V828T, D829G, Q830E, V842I, A844H, V846I, R863S, E877P, Q889M, F901Y, L915E, S941N, V967S, Q975D, H989F, T999N, K1080Q, S1146C, S1163L, L1168V, G1169P, S1177Q, V1194I, K1195R, D1197E, K1259H, Q1260K, S1281V, S1296E, H1315N, D1348N, and A1349G;
d) A14D, E28D, S33R, R44K, H45K, C84K, S91A, N92E, D99P, H117Y, E118S, D128E, R143I, K145Y, V147A, D151E, L162C, M165L, V185I, D186Q, I190K, Q194D, N206S, A207E, S208N, S217T, R219K, R224D, N228K, Q232N, E236Q, S249A, T253Q, S258T, A263E, D276E, D277E, D280E, Q289E, S301Y, N313D, E315T, I316N, S324A, Q334K, T337K, A341Q, L342F, A371T, F379Y, P382K, V395K, N398D, I418V, G421Q, R428E, R429N, E431G, D432K, F433Y, D439E, I447L, T470Q, S473T, E474D, E483D, G489E, A490K, Q493K, S494A, F502K, K510D, E520Q, Y521K, V534I, A542E, K550Q, A551E, D554E, L555H, T559K, N560Y, R561K, C578G, S585K, D609E, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711E, G740A, V747I, L751I, V754A, N762W, M785Y, Q798D, T808E, E813D, K814R, V828T, D829G, Q830E, V842I, A844H, V846I, K859R, R863S, D865K, E877K, Q889M, N892D, F901Y, L915E, S941E, D951G, L953R, V967S, Q975N, H989F, T999N, K1080Q, S1092N, T1142N, S1146C, S1163L, L1168V, G1169P, S1177K, E1193N, V1194I, K1195R, D1197E, S1234Q, K1259N, Q1260K, L1261K, S1281V, V1284H, D1292E, S1296Q, H1315N, D1348N, and A1349G;
e) A14D, E28D, S33R, H45K, C84K, N92D, D99P, H117Y, E118S, D128E, R143I, K145Y, V147A, D151Q, L162C, M165L, V185I, D186Q, I190K, Q194D, A207E, S208N, S217T, R219K, R224D, N228K, Q232N, E236Q, T253Q, A263E, D276E, D280E, Q285K, L295Q, S301Y, N313D, E315T, I316Q, Q334K, T337K, A341K, L342F, A371T, F379Y, P382K, V395K, N398D, I418V, G421Q, R428E, R429N, E431G, F433Y, D439E, I447L, S473T, E474D, E483D, G489E, A490K, Q493K, S494A, F502K, E520Q, Y521K, V534I, A542E, K550Q, A551Q, L555Y, T559K, N560Y, R561K, S585K, E617H, V624I, T626W, E637R, E638Q, K641S, T642K, K650E, V651Q, M652L, Q654R, K656E, S679N, K688I, S689D, A693N, T707S, D711I, G740A, L751I, V754A, N762W, Q798K, T808E, E813D, K814R, V828T, D829G, Q830E, V842I, A844H, V846I, R863S, E877K, Q889M, F901Y, L915E, S941N, V967S, Q975D, H989F, T999N, K1080S, F1084L, V1087I, S1146C, S1163L, L1168V, G1169P, S1177Q, E1193N, V1194I, K1195R, D1197E, S1234Y, K1259H, Q1260K, L1261K, S1281V, V1284H, S1296E, H1315N, D1348N, A1349G;
f) A14D, E28D, S33R, H45K, C84K, D99P, H117Y, E118S, D128E, R143I, K145Y, V147A, D151Q, L162V, M165L, V185I, D186Q, I190N, A207E, S208N, S217T, R219K, R224D, N228K, Q232N, E236Q, N244Q, T253Q, N259T, A263E, D276E, D280E, Q285K, S301Y, N313D, E315T, I316E, T337K, L342F, A371T, F379Y, V395E, N398D, R399K, G421Q, R428E, R429N, E431G, F433Y, D439K, I447L, S473T, E474D, E483D, G489E, A490K, Q493K, S494A, E520Q, Y521K, V534I, A542E, K550Q, A551E, L555Y, T559K, N560Y, S585K, E617H, V624I, T626W, E637R, E638Q, K641S, T642K, K650E, V651Q, M652L, Q654R, K656E, S679N, K688I, S689D, T707S, D711I, G740A, L751I, V754A, N762W, Q798K, T808E, E813D, V828T, D829G, Q830E, V842I, A844H, V846I, R863S, E877K, Q889M, F901Y, L915E, S941N, V967S, Q975D, H989F, T999N, K1080Q, S1146C, S1163L, L1168V, G1169P, S1177Q, V1194I, K1195R, D1197E, K1259H, Q1260K, S1281V, S1296E, H1315N, D1348N, A1349G;
g) K8R, A14D, E28D, S33R, H45K, C84K, S91A, N92D, D99P, S100N, K116R, H117G, E118S, D128E, R143I, K145Y, V147A, D151Q, L162I, M165L, V185I, D186Q, I190K, Q194D, A207E, S208N, S217T, R219K, R224D, N228K, Q232N, E236Q, T253Q, A263E, D276E, D280E, Q285K, S301Y, N313D, E315T, I316E, Q334K, T337K, A341Q, L342F, A371T, F379Y, P382K, V395E, N398D, R399K, I418V, G421Q, R428E, R429N, E431G, D432K, F433Y, D439E, I447L, S473T, E474D, E483D, G489E, A490K, Q493K, S494A, F502K, E520Q, Y521K, V534I, A542E, K550Q, A551Q, T559K, N560Y, R561K, S585K, E617H, V624I, T626W, E637R, E638Q, K641S, T642K, K650E, V651Q, M652L, Q654R, K656E, R668K, K688I, S689D, A693I, T707S, D711E, G740A, L751I, V754A, H758Q, N762S, Q798K, T808E, E813D, K814R, V828T, D829G, Q830E, V842I, A844H, V846I, R863S, D865K, E877K, Q889M, F901Y, L915E, S941E, L953R, Q975D, H989F, T999N, S1146C, S1163L, L1168V, G1169P, S1177K, E1193N, V1194I, K1195R, D1197E, S1234Q, N1238K, K1259H, Q1260K, L1261K, S1281V, V1284H, S1296E, H1315N, D1348N, and A1349G;
h) K8R, A14D, E28D, S33R, R44K, H45K, C84K, S91A, N92E, D99P, S100N, K116R, H117G, E118S, D128E, R143I, K145Y, V147A, D151E, L162C, M165L, V185I, D186Q, I190K, Q194D, N206S, A207E, S208N, S217T, R219K, R224D, N228K, Q232N, E236Q, S249A, T253Q, S258T, A263E, D276E, D280E, Q285K, S301Y, N313D, E315T, I316N, S324A, Q334K, T337K, A341Q, L342F, Q346Y, A371T, F379Y, P382K, V395K, N398D, I418V, G421Q, R428E, R429N, E431G, D432K, F433Y, D439E, I447L, T470Q, S473T, E474D, E483D, G489E, A490K, Q493K, S494A, F502K, E520Q, Y521K, V534I, A542E, K550Q, A551Q, L555Y, T559K, N560Y, R561K, K574R, S585K, E617H, V624I, T626W, E637R, E638Q, R639Q, K641S, T642K, H645D, D649E, K650E, V651Q, M652L, Q654R, K656E, R668K, S679N, K688I, S689D, A693N, T707S, D711I, G740A, V747I, L751I, V754A, N762W, M785Y, Q798D, T808E, E813D, K814R, V828T, D829G, V842I, A844H, V846I, K859R, R863S, D865K, E877K, Q889M, N892D, F901Y, L915E, S941E, D951G, Q975D, H989F, T999N, K1080Q, S1092N, T1142N, S1146C, S1163P, L1168V, G1169P, S1177K, E1193N, V1194I, K1195R, D1197E, S1234Q, N1238K, K1259N, Q1260K, L1261K, S1281V, V1284H, S1296E, H1315N, D1348N, and A1349G;
i) K8R, A14D, E28N, S33R, R44K, H45K, C84K, S91A, N92D, D99P, S100N, H117Y, E118S, D128E, R143I, K145Y, V147A, D151E, L162V, M165L, V185I, D186Q, I190N, Q194K, N206S, A207E, S208N, S217T, R219K, R224D, N228K, Q232N, E236Q, S249A, T253Q, S258T, A263E, D276E, D280E, Q285K, S301Y, N313D, E315T, I316N, S324A, Q334K, T337K, A341Q, L342F, Q346Y, A371T, F379Y, P382K, V395K, N398D, I418V, G421Q, R428E, R429N, E431G, D432K, F433Y, D439E, I447L, S473T, E474D, E483D, G489E, A490K, Q493K, S494A, F502K, E520Q, Y521K, V534I, A542E, K550Q, A551Q, D554E, L555Y, T559K, N560Y, R561K, S585K, E617H, V624I, T626W, E637R, E638Q, R639Q, K641S, T642K, H645D, D649E, K650E, V651Q, M652L, Q654R, K656A, R668K, K688I, S689D, A693N, T707S, D711I, G740A, V747I, L751I, V754A, H758Q, N762W, M785Y, Q798D, T808E, E813D, K814R, V828T, D829G, Q830E, V842I, A844H, V846I, K859R, R863S, D865K, E877K, Q889M, N892D, F901Y, L915E, S941E, D951G, L953R, V967S, Q975D, H989F, T999N, K1080Q, V1087I, S1092N, T1142N, S1146C, S1163P, L1168V, G1169P, S1177K, E1193N, V1194I, K1195R, D1197E, S1234Q, K1259H, Q1260K, L1261K, S1281V, V1284H, S1296E, H1315N, D1348N, and A1349G;
j) A14D, D27E, E28N, S33R, R44K, H45K, C84K, S91A, N92E, D99P, S100N, K116R, H117G, E118S, D128E, A131E, K135Q, R143I, K145Y, V147A, D151E, L162V, M165L, V185I, D186Q, I190N, Q198N, N206S, A207T, S208N, L216F, S217T, R219K, R224D, N228K, Q232N, E236Q, S249A, L252N, T253Q, S258K, A263E, D276E, D277E, D280E, Q285K, Q289E, L295N, S301Y, N313D, E315T, I316N, S324A, E331D, Q334K, T337K, A341Q, L342F, Q346Y, A371T, S372T, E375D, F379Y, E392Y, V395K, N398D, I418V, G421Q, R428E, R429N, E431G, D432K, F433Y, D439E, I447L, S473T, E474D, E483D, G489E, A490K, Q493K, S494A, F502K, K510D, E520Q, Y521K, V534I, A542E, K550Q, A551Q, L555H, T559K, N560Y, R561K, C578G, F579Y, S585K, D609E, N613D, E615K, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711I, Q713E, G740A, V747I, L751I, V754A, N762W, M785Y, Q798N, E806D, T808Q, E813D, K814R, V828T, D829G, Q830E, V842I, A844H, V846I, K859R, R863S, S864R, D865K, E877K, Q889M, N892E, F901Y, S913T, L915E, S941E, D951G, L953R, K965N, V967S, Q975N, H989F, T999N, K1080S, F1084L, V1087I, S1092N, N1119D, T1142N, S1146C, S1163L, L1168V, G1169P, S1177K, E1193N, V1194I, K1195R, D1197E, S1234Q, V1237I, L1242N, K1259N, Q1260K, L1261K, F1262Y, Q1265E, E1275N, S1281V, R1283K, V1284H, L1286G, D1288K, D1292E, L1295E, S1296K, H1315N, D1348N, and A1349G;
k) A14D, E28D, S33R, H45K, C84K, S91A, N92E, D99P, S100N, K116R, H117G, E118S, D128E, R143I, K145Y, V147A, D151Q, L162C, M165L, V185I, D186Q, I190K, Q194D, A207E, S208N, S217T, R219K, R224D, N228K, Q232N, E236Q, N244Q, T253Q, N259T, A263E, D276E, D280E, Q285K, S301Y, N313D, E315T, I316Q, Q334K, T337K, A341E, L342F, A371T, F379Y, P382K, V395E, N398D, R399K, I418V, G421Q, R428E, R429N, E431G, D432K, F433Y, D439E, I447L, S473T, E474D, G489E, A490K, Q493K, S494A, F502K, E520Q, Y521K, A542E, K550Q, A551Q, L555Y, T559K, N560Y, R561K, S585K, E617H, V624I, T626W, E637R, E638Q, K641S, T642K, K650E, V651Q, M652L, Q654R, K656E, R668K, S679N, K688I, S689D, A693I, T707S, D711E, G740A, L751I, V754A, H758Q, N762S, Q798K, T808E, E813D, K814R, V828T, D829G, V842I, A844H, V846I, R863S, D865K, E877K, Q889M, F901Y, L915E, S941E, L953R, V967S, Q975N, H989F, T999N, S1146C, S1163L, L1168V, G1169P, S1177K, E1193N, V1194I, K1195R, D1197E, S1234Q, K1259N, Q1260K, L1261K, S1281V, V1284H, S1296E, H1315N, D1348N, and A1349G;
l) K8R, A14D, E28D, S33R, H45K, C84K, D99P, H117Y, E118S, D128E, R143I, K145Y, V147A, D151K, L162I, V185I, D186Q, I190N, A207T, S208N, S217T, R219K, R224D, N228K, Q232N, E236Q, T253Q, D276E, D280E, Q285K, S301Y, N313D, E315T, I316Q, T337K, A341K, L342F, A371T, F379Y, V395K, N398D, G421Q, R428E, R429N, E431G, F433Y, D439K, I447L, S473T, E474D, E483D, G489E, A490K, Q493K, E520Q, Y521K, V534I, A542E, K550Q, A551Q, L555Y, T559N, N560Y, S585K, E617H, V624I, T626W, E637R, E638Q, K641S, T642K, K650E, V651Q, M652L, Q654R, S679N, K688I, S689D, T707S, D711I, G740A, L751I, V754A, N762W, Q798K, T808E, E813D, V828T, D829G, V842I, A844H, V846I, R863S, E877K, Q889M, F901Y, L915E, S941N, V967S, Q975D, H989F, T999N, K1080Q, S1146C, S1163L, L1168V, G1169P, S1177Q, V1194I, K1195R, D1197E, K1259H, S1281V, S1296E, D1348N, and A1349G;
m) A14D, E28N, S33R, R44K, H45K, C84K, S91A, N92E, D99P, H117Y, E118S, D128E, K135Q, R143I, K145Y, V147A, D151E, L162V, M165L, V185I, D186Q, I190K, Q194D, Q198N, N206S, A207E, S208N, L216F, S217T, R219K, R224D, N228K, Q232N, E236Q, S249A, L252N, T253Q, S258K, A263E, D276E, D277E, D280E, Q289E, L295N, S301Y, D308G, N313D, E315T, I316N, S324A, E331D, Q334K, T337K, A341Q, L342F, Q346Y, A371T, S372T, E375D, F379Y, P382K, E392Y, V395K, N398D, I418V, G421Q, R428E, R429N, E431G, D432K, F433Y, D439E, I447L, E474D, E483D, G489E, A490K, Q493K, S494A, F502K, K510D, E520Q, Y521K, V534I, A542E, K550Q, A551Q, L555H, T559K, N560Y, R561K, C578G, F579Y, S585K, D609E, N613D, E615K, E617H, V624I, T626W, E637R, E638Q, R639Q, K641S, T642K, A644S, H645D, D649E, K650E, V651Q, M652L, Q654R, K656A, R668K, S679N, K688I, S689D, A693N, T707S, D711I, Q713E, G740A, V747I, L751I, V754A, H758Q, N762W, M785Y, Q798N, E806D, T808Q, E813D, K814R, V828T, D829G, V842I, A844H, V846I, K859R, R863S, S864R, D865K, E877K, Q889M, N892E, F901Y, S913T, L915E, S941E, D951G, L953R, K965N, V967S, Q975D, H989F, T999N, F1084L, V1087I, S1092N, N1119D, T1142N, S1146C, S1163L, L1168V, G1169P, S1177K, E1193N, V1194I, K1195Q, D1197N, S1234Q, V1237I, N1238K, K1259H, Q1260K, L1261K, F1262Y, Q1265E, E1275N, S1281V, R1283K, V1284H, L1286G, D1292K, S1296Q, H1315N, D1348N, and A1349G; or
n) A14D, E28D, S33R, R44K, H45K, C84K, S91A, N92E, D99P, S100N, H117Y, E118S, D128E, R143I, K145Y, V147A, D151E, L162V, M165L, V185I, D186Q, I190K, Q194D, N206S, A207E, S208N, S217T, R219K, R224D, N228E, Q232N, E236Q, S249A, T253Q, S258T, A263E, D276E, D277E, D280E, Q285K, Q289E, S301Y, D308G, N313D, E315T, I316N, S324A, Q334K, T337K, A341Q, L342F, Q346Y, A371T, F379Y, V395K, N398D, I418V, G421Q, R428E, R429N, E431G, D432K, F433Y, D439E, I447L, T470Q, S473T, E474D, E483D, G489E, A490K, Q493K, S494A, F502K, K510D, E520Q, Y521K, V534I, A542E, K550Q, A551Q, D554E, L555H, T559K, N560Y, R561K, C578G, S585K, D609E, E617H, V624I, T626W, E637R, E638Q, R639Q, K641S, T642K, A644S, H645D, D649E, K650E, V651Q, M652L, Q654R, K656E, R668K, S679N, K688I, S689D, A693N, T707S, D711I, G740A, V747I, L751I, V754A, H758Q, N762W, M785Y, Q798D, T808E, E813D, K814R, V828T, D829G, Q830E, V842I, A844H, V846I, K859R, R863S, D865K, E877K, Q889M, N892E, F901Y, L915E, S941E, D951G, V967S, Q975N, H989F, T999N, K1080Q, V1087I, S1092N, T1142N, S1146C, S1163L, L1168V, G1169P, S1177K, E1193N, V1194I, K1195R, D1197E, S1234Q, L1242N, K1259N, Q1260K, L1261K, S1281V, V1284H, D1292E, S1296Q, H1315N, D1348N, and A1349G; or
2) Campylobacter jejuni of SEQ ID NO: 38 comprising mutations:
a) A6G, T309I, F398L, V404L, and T405I;
b) A6G, D117E, K155L, A157S, T303K, T309I, I366E, S384K, F398L, V404I, T405V, and N477H;
c) A6G, T309I, I366E, F398L, V404I, T405V, and N477H;
d) A6G, T309I, F398L, V404I, T405I, and N477H;
e) A6G, D117E, T309I, I366E, S384K, F398L, V404I, T405V, and N477H;
f) A6G, D117E, K155L, T309I, I366E, S384K, F398L, V404I, T405V, and N477H;
g) A2K, A6G, D117E, K155L, A157S, S228E, K288D, A293K, T303K, T309I, I366D, K367E, S384K, F398L, L403K, V404I, T405V, L409E, E410Q, and N477H, or
h) A6G, D117E, K155L, T309I, I366E, S384K, F398L, V404I, T405I, and N477H.

268. (canceled)

269. A nucleic acid encoding the Cas9 variant of claim 267.

270. An expression vector comprising the nucleic acid of claim 269.

271. A host cell transformed with the expression vector of claim 270.

272. A method for producing a Cas9 variant, said method comprising expressing the Cas9 variant in the transformed host cell of claim 271.

273. A method for genome editing, said method comprising:
a) designing a guide RNA (gRNA) targeting sequence complementary to a specific DNA target in a double stranded DNA, wherein the specific DNA target is located in close proximity to a target gene to be edited and is located upstream of a Protospacer Adjacent Motif (PAM) sequence, wherein the PAM sequence is a binding site for Cas9;
b) coupling the gRNA to a Cas9 variant of claim 267 to form a gRNA-Cas9 variant complex;
c) delivering to a target cell the gRNA-Cas9 variant complex in combination with exogenous donor DNA, wherein the gRNA guides the Cas9 variant to the specific DNA target, wherein (i) PAM sequence recognition by the Cas9 variant and binding therewith identifies the specific DNA target and (2) base pairing between the gRNA and the specific DNA target activate a Cas9 nuclease site on each strand of the double-stranded DNA to blunt-end cleave the double-stranded DNA at each nuclease site forming double stranded breaks, said breaks located 3-4 nucleotides downstream from the PAM sequence,
wherein the exogenous donor DNA encodes a desired protein and is flanked by nucleotide sequences identical to nucleotide sequences framing the specific DNA target;
wherein generation of the blunt-end double-stranded cleavage in the specific DNA target activates an endogenous DNA repair pathway; and
wherein the Cas9 variant has a binding and cleavage specificity that is increased over the binding and cleavage specificity of a Cas9 which does not comprise the mutations of the Cas9 variant.

274. The method of claim 273, wherein the endogenous DNA repair pathway is homologous recombination (HR).

275. The method of claim 274, wherein the HR results in a knock-in of the donor DNA at the specific DNA target site.

276. The method of claim 273, wherein the endogenous DNA repair pathway is non-homologous end-joining (NHEJ), wherein said NHEJ either inserts or deletes nucleotides at the specific DNA target site, resulting in a knock-out of the target gene.

277. The method of claim 273, wherein the Cas9 variant cleaves only a single strand of the double-stranded DNA.

278. The method of claim 273, wherein the target cell is cultured in vitro.

279. The method of claim 273, wherein the gRNA-Cas9 variant complex is delivered by transient transfection, cytoplasmic injection or nuclear injection.

280. The method of claim 273, wherein the gRNA and the Cas9 variant are delivered by transient transfection of an expression vector comprising a gene encoding the variant Cas9 and a gene encoding the gRNA; cytoplasmic injection or nuclear injection of said expression vector or of mRNA encoding the variant Cas9 and of mRNA of the gRNA, or transduction of the target cell with a virus expressing a gene encoding the variant Cas9 and expressing a gene encoding the gRNA.

281. A method for treating a subject having a deficiency of an enzyme, said method comprising administering to the subject target cells expressing a gene edited by the method of claim 273, wherein edited gene comprises a donor DNA encoding the enzyme.

282. A method for modifying a food, said method comprising treating the food with bacteria expressing a gene edited by the method of claim 273, wherein edited gene comprises a donor DNA encoding a gene for immunity against a virus.

283. The method of claim 282, wherein the food is yogurt or cheese.

284. A method for producing a disease-resistant grain, said method comprising modifying the DNA of the grain to encode a gene edited by the method of claim 273, wherein the edited gene comprises a donor DNA encoding a gene for immunity against the disease.

285. A method for producing a disease-resistant livestock, said method comprising modifying the DNA of the livestock to encode a gene edited by the method of claim 273, wherein the edited gene comprises a donor DNA encoding a gene for immunity against the disease.

286. A method for producing a biopharmaceutical, said method comprising modifying the DNA of a bacterium to encode a gene edited by the method of claim 273, wherein the edited gene comprises a donor DNA encoding a gene for a biopharmaceutical.


SEQUENCE LISTING (38 sequences)

SEQ ID NO: 1 - protein, 659 aa, Bacillus amyloliquefaciens
MFEKRFKTSLLPLFAGFLLLFHLVLSGPAAANAETANKSNEVAASSVKNGTILHAWNWSFNTLTQNMKEIRDAGYAAIQTSPINQVKEGNQGDKSMRNWYWLYQPTSYQIGNRYLGTEQEFKDMCAAAEKYGVKVIVDAVINHTTSDYAAISDEIKRIPNWTHGNTQIKNWSDRWDVTQNSLLGLYDWNTQNTEVQTYLKRFLERALNDGADGFRYDAAKHIELPDDGNYGSRFWPNITNTSAEFQYGEILQDSASRDTAYANYMNVTASNYGHSIRSALKNRNLSVSNISHYASDVSADKLVTWVESHDTYANDEEESTWMSDDDIRLGWAVIGSRSGSTPLFFSRPEGGGNGVRFPGKSQIGDRGSALFKDQAITAVNQFHNVMAGQPEELSNPNGNNQIFMNQRGSKGVVLANAGSSSVTVNTSTKLPDGRYDNRAGAGSFQVANGKLTGTINARSAAVLYSDDIGNAPHVFLENYQTGAVHSFNDQLTVTLRANAKTAKAVYQINNGQQTAFKDGDRLTIGKGDPIGTTYNIRLTGTNGEGAERTQEYTFVKKDQAQTNIIGYQNPDHWGQVNAYIYKHDGGRAIELTGSWPGKAMTKNANGIYTLTLPANADTANAKVIFNNGSAQVPGQNQPGFDYVQNGLYNNSGLNGYLPH

SEQ ID NO: 2 - protein, 659 aa, Bacillus licheniformis
MFQKRFKTSLLPLFAGFLLLFHMVMAGPAAANAETQNTSNELTAPSIKSGTILHAWNWSFNTLKHNMKDIHDAGYTAIQTSPINQVKEGNQGNKSMSNWYWLYQPTSYQIGNRYLGTEQEFKEMCAAAEEYGVKVIVDAVINHTTSDYAAISNEIKSIPNWTHGNTQIKNWSDRWDVTQNSLLGLYDWNTQNTQVQSYLKRFLETALNDGADGFRFDAAKHIELPDDGNYGSQFWPNITNTSAEFQYGEILQDSASRDAAYANYMNVTASNYGHSIRSALKNRNLSVSNISHYASDVSADKLVTWVESHDTYANDDEESTWMSDDDIRLGWAVIASRSGSTPLFFSRPEGGGNGVRFPGKTQIGDRGSALFEDQAIAAVNTFHNVMAGQPEELSNPNGNNQIFMNQRGSQGIVLANAGSSSVSINVSTKLPDGSYDNKAGAGSFQVRDGKLTGTLNARSVAVLYPDDIANAPQVSLENYKTGVTHFFNDQLTITLRANANTTKAVYQINNGQETAFKDGDQLTIGKGDPFGKTYNITLTGTNSDGVTRTQEYSFVKRDPSSAKTIGYQNPNHWGQVNAYIYKHDGGRAIELTGSWPGKAMTKNADGIYTLTLPADTDTTNAKVIFNNGSAQVPGQNQPGFDYVQNGLYNDSGLSGSLPQ

SEQ ID NO: 3 - protein, 535 aa, Barley
MEVNVKGNYVQVYVMLPLDAVSVNNRFEKGDELRAQLRKLVEAGVDGVMVDVWWGLVEGKGPKAYDWSAYKQLFELVQKAGLKLQAIMSFHQCGGNVGDAVNIPIPQWVRDVGTRDPDIFYTDGHGTRNIEYLTLGVDNQPLFHGRSAVQMYADYMTSFRENMKEFLDAGVIVDIEVGLGPAGEMRYPSYPQSHGWSFPGIGEFICYDKYLQADFKAAAAAVGHPEWEFPNDAGQYNDTPERTQFFRDNGTYLTEKGRFFLAWYSNNLIKHGDRILDEANKVFLGYKVQLAIKISGIHWWYKVPSHAAELTAGYYNLHDRDGYRTIARMLKRHRASINFTCAEMRDSEQSSQAMSAPEELVQQVLSAGWREGLNVACENALPRYDPTAYNTILRNARPHGINQSGPPEHKLFGFTYLRLSNQLVEGQNYVNFKTFVDRMHANLPRDPYVDPMAPLPRSGPEISIEMILQAAKPKLQPFPFQEHTDLPVGPTGGMGGQAEGPTCGMGGQVKGPTGGMGGQAEDPTSGMGGELPATM

SEQ ID NO: 4 - protein, 499 aa, Sweet potato
MAPIPGVMPIGNYVSLYVMLPLGVVNADNVFPDKEKVEDELKQVKAGGCDGVMVDVWWGIIEAKGPKQYDWSAYRELFQLVKKCGLKIQAIMSFHQCGGNVGDAVFIPIPQWILQIGDKNPDIFYTNRAGNRNQEYLSLGVDNQRLFQGRTALEMYRDFMESFRDNMADFLKAGDIVDIEVGCGAAGELRYPSYPETQGWVFPGIGEFQCYDKYMVADWKEAVKQAGNADWEMPGKGAGTYNDTPDKTEFFRPNGTYKTDMGKFFLTWYSNKLIIHGDQVLEEANKVFVGLRVNIAAKVSGIHWWYNHVSHAAELTAGFYNVAGRDGYRPIARMLARHHATLNFTCLEMRDSEQPAEAKSAPQELVQQVLSSGWKEYIDVAGENALPRYDATAYNQMLLNVRPNGVNLNGPPKLKMSGLTYLRLSDDLLQTDNFELFKKFVKKMHADLDPSPNAISPAVLERSNSAITIDELMEATKGSRPFPWYDVTDMPVDGSNPFD

SEQ ID NO: 5 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 6 - protein, 714 aa, Bacillus macerans
MKSRYKRLTSLALSLSMALGISLPAWASPDTSVDNKVNFSTDVIYQIVTDRFADGDRTNNPAGDAFSGDRSNLKLYFGGDWQGIIDKINDGYLTGMGVTALWISQPVENITSVIKYSGVNNTSYHGYWARDFKQTNDAFGDFADFQNLIDTLTLITSRSDRLRPQPHVSGRAGTNPGFAENGALYDNGSLLGAYSNDTAGLFHHNGGTDFSTIEDGIYKNLYDLADINHNNNAMDAYFKSAIDLWLGMGVDGIRFDAVKQYPFGWQKSFVSSIYGGDHPVFTFGEWYLGADQTDGDNIKFANESGMNLLDFEYAQEVREVFRDKTETMKDLYEVLASTESQYDYINNMVTFIDNHDMDRFQVAGSGTRATEQALALTLTSRGVPAIYYGTEQYMTGDGDPNNRAMMTSFNTGTTAYKVIQALAPLRKSNPAIAYGTTTERWVNNDVLIIERKFGSSAALVAINRNSSAAYPISGLLSSLPAGTYSDVLNGLLNGNSITVGSGGAVTNFTLAAGGTAVWQYTAPETSPAIGNVGPTMGQPGNIVTIDGRGFGGTAGTVYFGTTAVTGSGIVSWEDTQIKAVIPKVAAGKTGVSVKTSSGTASNTFKSFNVLTGDQVTVRFLVNQANTNYGTNVYLVGNAAELGTWDPNKAIGPMYNQVIAKYPSWYYDVSVPAGTKLDFKFIKKGGGTVTWEGGGNHTYTTPASGVGTVTVDWQN

SEQ ID NO: 7 - protein, 711 aa, Geobacillus stearothermophilus
MKRWLSVVLSMSLVFSAFFLVSDTQKVTVEAAGNLNKVNFTSDIVYQIVVDRFVDGNTSNNPSGSLFSSGCTNLRKYCGGDWQGIINKINDGYLTEMGVTAIWISQPVENVFAVMNDADGSTSYHGYWARDFKKTNPFFGTLSDFQRLVDAAHAKGIKVIIDFAPNHTSPASETNPSYMENGRLYDNGTLIGGYTNDTNSYFHHNGGTTFSNLEDGIYRNLFDLADFNHQNQFIDKYLKDAIKLWLDMGIDGIRMDAVKHMPFGWQKSFMDEVYDYRPVFTFGEWFLSENEVDSNNHFFANESGMSLLDFRFGQKLRQVLRNNSDDWYGFNQMIQDTASAYDEVIDQVTFIDNHDMDRFMADEGDPRKVDIALAVLLTSRGVPNIYYGTEQYMTGNGDPNNRKMMTSFNKNTRAYQVIQKLSSLRRSNPALSYGDTEQRWINSDVYIYERQFGKDVVLVAVNRSLSKSYSITGLFTALPSGTYTDQLGALLDGNTIQVGSNGAVNAFNLGPGEVGVWTYSAAESVPIIGHIGPMMGQVGHKLTIDGEGFGTNVGTVKFGNTVASVVSWSNNQITVTVPNIPAGKYNITVQTSGGQVSAAYDNFEVLTNDQVSVRFVVNNANTNWGENIYLVGNVHELGNWNTSKAIGPLFNQVIYSYPTWYVDVSVPEGKTIEFKFIKKDGSGNVIWESGSNHVYTTPTSTTGTVNVNWQY

SEQ ID NO: 8 - protein, 327 aa, Bacillus amyloliquefaciens
MKDMVRERLEKEELASLLQDWVEHYRPFSVNGKSANYIPALGEINDSQLGICILEPDGSMIHAGEWNVPFTLQSISKVISFIAACMSRGIPYVLDRVDVEPTGDAFNSIIRLEIHKPGKPFNPMINAGAITVASLLPGETAHNKLEYLFSVFEKLIGKRPHIHEEVFRSEWQTAHRNRALAYYLKETNFLEADVEETLEVYLKQCAMESTTEDIALIGLILAHDGYHPIRREQVIPKDVAKLAKALMLTCGMYNASGKYAAFVGVPAKSGVSGGIMALVPPSARREQPFHNGCGIGIYGPAIDEYGNSLTGGMLLKHIAQEWELSIF

SEQ ID NO: 9 - protein, 422 aa, Bacillus amyloliquefaciens
MTAVNQTISKVVNGKRVITKKPELLAPAGNLEKLKIAVHYGADAVFIGGQEYGLRSNADNFSIEEIAEGVEFAKKYGAKIYVTTNIFAHNENMDGLEEYLKALGDAKVAGIIVADPLIIETCRRVAPDVEIHLSTQQSLSNWKAVQFWKEEGLDRVVLARETSGLEIKEMKEKVDIEIETFIHGAMCIAYSGRCVLSNHMTARDSNRGGCCQSCRWDYDLYQTDGANAVALYDEEDAPFAMSPKDLKLIESIPQMIEMGIDSLKIEGRMKSIHYVATVVSVYRKVIDAYCADPENFVIQKEWLDELDKCANRDTAPAFFEGTPGYEEQMFGEHGKKTTFDFAGLVLAYDEETQMVTLQQRNFFKQGDEVEFFGPEIDNFTFTIGTIWDEDGNELDAARHPLQIVRFKVDKKIYPSNMMRKGK

SEQ ID NO: 10 - protein, 718 aa, Bacillus sp.
MVSIRRSFEAYVDDMNIITVLIPAEQKEIMTPPFRLETEITDFPLAVREEYSLEAKYKYVCVSDHPVTFGKIHCVRASSGHKTDLQIGAVIRTAAFDDEFYYDGELGAVYTADHTVFKVWAPAATSAAVKLSHPNKSGRTFQMTRLEKGVYAVTVTGDLHGYEYLFCICNNSEWMETVDQYAKAVTVNGEKGVVLRPDQMKWTAPLKPFSHPVDAVIYETHLRDFSIHENSGMINKGKYLALTETDTQTANGSSSGLAYVKELGVTHVELLPVNDFAGVDEEKPLDAYNWGYNPLHFFAPEGSYASNPHDPQTRKTELKQMINTLHQHGLRVILDVVFNHVYKRENSPFEKTVPGYFFRHDECGMPSNGTGVGNDIASERRMARKFIADCVVYWLEEYNVDGFRFDLLGILDIDTVLYMKEKATKAKPGILLFGEGWDLATPLPHEQKAALANAPRMPGIGFFNDMFRDAVKGNTFHLKATGFALGNGESAQAVMHGIAGSSGWKALAPIVPEPSQSINYVESHDNHTFWDKMSFALPQENDSRKRSRQRLAAAIILLAQGVPFIHSGQEFFRTKQGVENSYQSSDSINQLDWDRRETFKEDVHYIRRLISLRKAHPAFRLRSAADIQRHLECLTLKEHLIAYRLYDLDEVDEWKDIIVIHHASPDSVEWRLPNDIPYRLLCDPSGFQEDPTEIKKTVAVNGIGTVILYLASDLKSFA

SEQ ID NO: 11 - protein, 1102 aa, Klebsiella pneumoniae
MLRYTCHALFLGSLVLLSGCDNSSSSSTSGSPGSPGNPGNPGTPGTPDPQDVVVRLPDVAVPGEAVQASARQAVIHLVDIAGITSSTPADYATKNLYLWNNETCDALSAPVADWNDVSTTPTGSDKYGPYWVIPLTKESGCINVIVRDGTNKLIDSDLRVSFSDFTDRTVSVIAGNSAVYDSRADAFRAAFGVALADAHWVDKTTLLWPGGENKPIVRLYYSHSSKVAADSNGEFTDKYVKLTPTTVSQQVSMRFPHLASYPAFKLPDDVNVDELLQGETVAISAESDGILSSATQVQTAGVLDDTYAAAAEALSYGAQLTDSGVTFRVWAPTAQQVELVVYSADKKVVASHPMTRDSASGAWSWQGGSDLKGAFYRYAMTVYHPQSRKVEQYEVTDPYAHSLSTNSEYSQVVDLNDSALKPEGWDGLTMPHAQKTKADLAKMTIHESHIRDLSAWDQTVPAELRGKYLALTAQESNMVQHLKQLSASGVTHIELLPVFDLATVNEFSDKVADIQQPFSRLCEINSAVKSSEFAGYCDSGSTVEEVLTQLKQNDSKDNPQVQALNTLVAQTDSYNWGYDPFHYTVPEGSYATDPEGTARIKEFRTMIQAIKQDLGMNVIMDVVYNHTNAAGPTDRTSVLDKIVPWYYQRLNETTGSVESATCCSDSAPEHRMFAKLIADSLAVWTTDYKIDGFRFDLMGYHPKAQILSAWERIKALNPDIYFFGEGWDSNQSDRFEIASQINLKGTGIGTFSDRLRDAVRGGGPFDSGDALRQNQGVGSGAGVLPNELTTLSDDQARHLADLTRLGMAGNLADFVLIDKDGAVKKGSEIDYNGAPGGYAADPTEVVNYVSKHDNQTLWDMISYKAAQEADLDTRVRMQAVSLATVMLGQGIAFDQQGSELLRSKSFTRDSYDSGDWFNRVDYSLQDNNYNVGMPRSSDDGSNYDIIARVKDAVATPGETELKQMTAFYQELTALRKSSPLFTLGDGATVMKRVDFRNTGTDQQTGLLVMTIDDGMQAGASLDSRVDGIVVAINAAPESRTLQDFAGTSLQLSAIQQAAGDRSLASGVQVAADGSVTLPAWSVAVLELPQGESQGAGLPVSSK

SEQ ID NO: 12 - protein, 410 aa, Streptomyces mobaraensis
MSQRGRTLVFAALGAVMCTTALMPSAGAATGSGSGSGTGEEKRSYAETHRLTADDVDDINALNESAPAASSAGPSFRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 13 - protein, 517 aa, Alteromonas sp.
MNKLAVLYAEHIATLQKRTREIIERENLDGVVFHSGQAKRQFLDDMYYPFKVNPQFKAWLPVIDNPHCWIVANGTDKPKLIFYRPVDFWHKVPDEPNEYWADYFDIELLVKPDQVEKLLPYDKARFAYIGEYLEVAQALGFELMNPEPVMNFYHYHRAYKTQYELACMREANKIAVQGHKAARDAFFQGKSEFEIQQAYLLATQHSENDNAYGNIVALNENCAILHYTHFDRVAPATHRSFLIDAGANFNGYAADITRTYDFTGEGEFAELVATMKQHQIALCNQLAPGKLYGELHLDCHQRVAQTLSDFNIVDLSADEIVAKGITSTFFPHGLGHHIGLQVHDVGGFMADEQGAHQEPPEGHPFLRCTRKIEANQVFTIEPGLYFIDSLLGDLAATDNNQHINWDKVAELKPFGGIRIEDNIIVHEDSLENMTRELRARLTTHSLRGLSAPQFSINDPAVMSEYSYPSEPLSYEEEIKKSTFIVHVRTRRILVRRRTLSPILIAVTPMPAITAGLM

SEQ ID NO: 14 - protein, 327 aa, Aspergillus niger [sequence omitted]

SEQ ID NO: 15 - protein, 213 aa, Bacillus amyloliquefaciens
MFKFKKKFLVGLTAAFMSISMFSATASAAGTDYWQNWTDGGGTVNAVNGSGGNYSVNWSNTGNFVVGKGWTTGSPFRTINYNAGVWAPNGNGYLTLYGWTRAPLIEYYVVDSWGTYRPTGTYKGTVKSDGGTYDIYTTTRYNAPSIDGDNTTFTQYWSVRQSKRPTGSNAAITFSNHVNAWKSHGMNLGSNWAYQVLATEGYKSSGSSNVTVW

SEQ ID NO: 16 - protein, 353 aa, Penicillium
MIPNITQLKTAALVMLFAGQALSGPVESRQASESIDAKFKAHGKKYLGNIADQGTLNGNPKTPAIIKANFGQLSPENSMKWDATEPSQGQFSFAGSDYFVEFAETNGKLIRGHTLVWHSQLPSWVSSITDKTTLTDVMKNHITTVMKQYKGKLYAWDVVNEIFEEDGTLRDSVFSRVLGEDFVRIAFETAREADPEAKLYINDYNLDSATSAKLQGMVSHVKKWIAAGVPIDGIGSQTHLGAGAGAAASGALNALASAGTEEVAVTELDIAGATSTDYVDVVNACLDQPKCVGITVWGVADPDSWRADESPLLFDASYNPKEAYNVSQLLSRQHAFDLYLKLGNLLLSRLHSD

SEQ ID NO: 17 - protein, 585 aa, Thermoactinomyces
MLLEAIFHEAKGSYAYPISETQLRVRLRAKKGDVVRCEVLYADRYASPEEELAHALAGKAGSDERFDYFEALLECSTKRVKYVFLLTGPQGEAVYFGETGFSAERSKAGVFQYAYIHRSEVFTTPEWAKEAVIYQIFPERFANGDPSNDPPGTEQWAKDARPRHDSFYGGDLKGVIDRLPYLEELGVTALYFTPIFASPSHHKYDTADYLAIDPQFGDLPTFRRLVDEAHRRGIKIILDAVFNHAGDQFFAFRDVLQKGEQSRYKDWFFIEDFPVSKTSRTNYETFAVQVPAMPKLRTENPEVKEYLFDVARFWMEQGIDGWRLDVANEVDHAFWREFRRLVKSLNPDALIVGEIWHDASGWLMGDQFDSVMNYLFRESVIRFFATGEIHAERFDAELTRARMLYPEQAAQGLWNLLDSHDTERFLTSCGGNEAKFRLAVLFQMTYLGTPLIYYGDEIGMAGATDPDCRRPMIWEEKEQNRGLFEFYKELIRLRHRLASLTRGNVRSWHADKQANLYAFVRTVQDQHVGVVLNNRGEKQTVLLQVPESGGKTWLDCLTGEEVHGKQGQLKLTLRPYQGMILWNGR

SEQ ID NO: 18 - protein, 463 aa, Trichoderma reesei
MAPSATLPLTTAILAIGRLVAAQQPGTSTPEVHPKLTTYKCTTSGGCVAQDTSVVLDWNYRWMHDANYNSCTVNGGVNTTLCPDEATCGKNCYIEGVDYAASGVTASGSTLTLNQYMPSSSGGYSSVSPRLYLLGPDGEYVMLKLNGQELSFDVDLSALPCGENGSLYLSQMDENGGANQYNTAGANYGSGYCDAQCPVQTWRNGTLNTSGQGFCCNEMDILEGNSRANALTPHSCTATACDSAGCGFNPYGSGYPNYFGPGDTVDTSKTFTIITQFNTDNGSPSGNLVSITRKYRQNGVDIPSAKPGGDTISSCPSASAYGGLATMGKALSSGMVLVFSIWNDNSQYMNWLDSGRAGPCSSTEGNPSNILANNPGTHVVYSNIRWGDIGSTTNSTGGNPPPPPPPASSTTFSTTRRSSTTSSSPSCTQTHWGQCGGIGYTGCKTCTSGTTCQYGNDYYSQCL

SEQ ID NO: 19 - protein, 493 aa, Bacillus megaterium
MKRSISIFITCLLMTVLTISGFAVPEASAAGAKTPVALNGQLSIKGTQLVNQNGKPVQLKGISSHGLQWFGDYVNKDTLKWLRDDWGITVFRAAMYTADGGYIENPSVKNKVKEAVEAAKELGIYVIIDWHILNDGNPNQNKEKAKEFFKEMSSLYGSSPNVIYEIANEPNGDVNWKRDIKPYAEEVISVIRKNDPDNIIITGTGTWSQDVNDAADDQLKDANVMYALHFYAGTHGQFLRDKADYALSKGAPIFVTEWGTSDASGNGGVYLDQSREWLNYLDSKKISWVNWNLSDKQESSSALKPGASKTGGWPLSDLSASGTFVRENIRGSQNSREDRSETPKQEKPAQENSISVQYRTGDGSVNSNQIRPQINVKNNSKTTVNLKNVTVRYWYNTKNKGQNFDCDYAKIGCSNVTHKFVTLHKPVKGADAYLELGFRNGTLSPGASTGEIQIRLHNEDWSNYSQAGDYSFFQSNTFKDTKKITLYHQGKLI

SEQ ID NO: 20 - protein, 388 aa, Streptomyces marinus
MSFQPTPEDRFTFGLWTVGWQGRDPFGDATRPALDPVETVQRLAELGAYGVTFHDDDLIPFGSSDTERESHIKRFRQALDATGMTVPMATTNLFTHPVFKDGGFTANDRDVRRYALRKTIGNIDLAAELGAKTYVAWGGREGAESGGAKDVRDALDRMKEAFDLLGEYVTAQGYDLRFAIEPKPNEPRGDILLPTVGHALAFIERLERPELYGVNPEVGHEQMAGLNFPHGIAQALWAGKLFHIDLNGQSGIKYDQDLRFGAGDLRAAFWLVDLLETAGYEGPRHFDFKPPRTEDFDGVWASAAGCMRNYLILKDRAAAFRADPEVQEALRAARLDQLAQPTAADGLDALLADRAAFEDFDVDAAAARGMAFEHLDQLAMDHLLGARG

SEQ ID NO: 21 - protein, 516 aa, Aspergillus niger
MLNPKVAYMVWMTCLGLTLPSQAQSNDYRPSYHFTPDQYWMNEPNGLIKIGSTWHLFFQHNPTANVWGNICWGHATSTDLMHWAHKPTAIADENGVEAFTGTAYYDPNNTSGLGDSANPPYLAWFTGYTTSSQTQDQRLAFSVDNGATWTKFQGNPIISTSQEAPHDITGGLESRDPKVFFHRQSGNWIMVLAHGGQDKLSFWTSADTINWTWQSDLKSTSINGLSSDITGWEVPDMFELPVEGTEETTWVVMMTPAEGSPAGGNGVLAITGSFDGKSFTADPVDASTMWLDNGRDFDGALSWVNVPASDGRRIIAAVMNSYGSNPPTTTWKGMLSFPRTLSLKKVGTQQHFVQQPITELDTISTSLQILANQTITPGQTLLSSIRGTALDVRVAFYPDAGSVLSLAVRKGASEQTVIKYTQSDATLSVDRTESGDISYDPAAGGVHTAKLEEDGTGLVSIRVLVDTCSVEVFGGQGEAVISDLIFPSDSSDGLALEVTGGNAVLQSVDVRSVSLE

SEQ ID NO: 22 - protein, 1025 aa, Kluyveromyces
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

SEQ ID NO: 23 - protein, 147 aa, Chicken
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 24 - protein, 379 aa, Aspergillus niger
MHYKLLFAAAAASLASAVSAAGVVGAAEGFAHGVTGGGSASPVYPTTTDELVSYLGDNEPRVIILDRTFDFTGTEGTETTTGCAPWGTASQCQVAINLHSWCDNYQASAPKVSVTYDKAGILPITVNSNKSIVGQGTKGVIKGKGLRVVSGAKNVIIQNIAVTDINPKYVWGGDAITVDDSDLVWIDHVTTARIGRQHIVLGTSADNRVTISYSLIDGRSDYSATCNGQHYWGVYLDGSNDMVTLKGNYFYNLSGRMPKVQGNTLLHAVNNLFHNFDGHAFEIGTGGYVLAEGNVFQDVNTVVETPISGQLFSSPDANINQQCASVFGRSCQLNAFGNSGSMSGSDTSIISKFAGKTIATAHPPGNIAQWTMKNAGQGK

SEQ ID NO: 25 - protein, 432 aa, Escherichia coli
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIMAALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 26 - protein, 446 aa, Buttiauxella sp.
MTISAFERQKLTLRNGLLIAIGTACSLGSTVYASETPASGYQTEKVVILSRHGVRAPTKMTQTMRDVTPNTWPEWPVKLGYITPRGEQLIRVMGGFYREKFQQQGILSQENCPAPNSVYVWADVDQRTLKTGEAFLAGLAPQCGLTIHHQQNLEKADPLFHPVKAGICSMEKNQIQQAVEKEAQNPIDNLNQHYLPSLALMNTTLNFANSAWCQKHSVDKSCDLGQSMPSKLSIQENGNKITLDGAIGLSSTLAEIFLLEYAQGMPKAAWGNIHSEQEWAALLKLHNTQFDLMARTPYIARHNGTPLLQAISNALDPNATASKLPDISPDNKILFIAGHDTNIANISGMLNISWTLPGQPDNTPPGGALVFERLADKSGKQYVSVSMVYQTLEQLHAQTPLSLKEPAGSVQLKIPGCSDQTAEGYCPLPTFTRMVSQSVEPGCQLQ

SEQ ID NO: 27 - protein, 478 aa, Aspergillus niger
MHSYQLLGLAAVGSLVSAAPAPSRVSEFAKKASTCTFTSASEASESISSCSDVVLSGIEVPAGETLDLSDAADGSTITFEGTTSFGYKEWKGPLIRFGGKDLTVTMADGAVIDGDGSRWWDSKGTNGGKTKPKFMYIHDVEDSTFKGINVKNTPVQAISVQATNVHLNDFTIDNSDGDDNGGHNTDGFDISESTGVYISGATVKNQDDCIAINSGESISFTGGTCSGGHGLSIGSVGGRDDNTVKNVTISDSTVSDSANGVRIKTIYKETGDVSEITYSNIQLSGITKYGIVIEQDYENGSPTGTPSTGIPITDVTVDGITGTLEDDATQVYILCGDGSCSDWTWSGVDLSGGKASDDCENVPSRFLHHCCSPPSRDSKFDLTFLVHNGEYPSAVILENKIVSKHWSDEERSVTATDNEENTVEASHYTDEGYTNVTLTEARSFKAQDVSRARQEILRFETRRLTHDNDMLIAMLQSE

SEQ ID NO: 28 - protein, 196 aa, Saccharomyces cerevisiae
MKQIVKRSHAIRIVAALGIIGLWMFFSSNELSIATPGLIKAKSGIDEVQGAAAEKNDARLKEIEKQTIMPLMGDDKVKKEVGRASWKYFHTLLARFPDEPTPEEREKLHTFIGLYAELYPCGECSYHFVKLIEKYPVQTSSRTAAAMWGCHIHNKVNEYLKKDIYDCATILEDYDCGCSDSDGKRVSLEKEAKQHG

SEQ ID NO: 29 - protein, 233 aa, Talaromyces pinophilus
MVAFSSLFTAFTAIAGSLAAPADLVKNDNVPDLVSRGPSNFVIDSTNNTSLARRSAINYDQDYTTGGTVNYYSSSTGFEVTWNTQDDFVVGVGWNPGSTTPINFGGQFSVSSGTGLLSVYGWSTSPLVEYYIIEDYANPPSFGTQKGSLTSDGGTYIIWENTRYNEPSIQGTSTFNQYISVRQSPRTEGTVTVQNHFNAWKNLGMNLGTLNFQVIAAEGWGGSGSASYAVNNN

SEQ ID NO: 30 - protein, 225 aa, Thermomyces lanuginosus
MVGFTPVALAALAATGALAFPAGNATELEKRQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 31 - protein, 420 aa, Bacillus paralicheniformis
MKKLICVLLAYVTMLSLMLTGPAASEVSAASDATVRLSAEKQVIRGFGGMNHPAWIGDLTAAQRETAFGNGQNQLGFSILRIHVDENRNNWYREVETAKSAIKHGAIVFASPWNPPSDMVETFNRNGDTSAKRLRYDKYAAYAKHLNDFVTFMKNNGVNLYAISVQNEPDYAHDWTWWTPQEILRFMKENAGSINARVIAPESFQYLKNISDPILNDPKALANMDILGAHLYGTQLNNFAYPLFKQKGAGKDLWMTEVYYPNSDNNSADRWPEALDVSHHIHNSMVEGDFQAYVWWYIRRSYGPMKEDGTISKRGYNMAHFSKFVRPGYVRVDATKSPASNVYVSAYKGDNKVVIVAINKNNSGVNQNFVLQNGSVSQVSRWITSSSSNLQPGTNLNVTDNHFWAHLPAQSVTTFVANLR

SEQ ID NO: 32 - protein, 228 aa, Actinomadura
MIQAPARPKSRRRVRARLLASGVSAVAMVAATTVLPGTAHAAITTNQTGWHNGYFYSFWTDSQGTVSMELGSGGNYSTSWYNTGNFVAGKGWNPGGRRTVNYSASYSPSGNSYLTLYGWTRNPLVEYYIVDSWGSWRPTGTYKGTITSDGGTYDIYQTMRYNAPSIEGIRTFPQYWSVRQSKRTSGTITSGNHFDAWARYGMNLGSHDYMIMATEGYQSSGYSNVTVW

SEQ ID NO: 33 - protein, 327 aa, Aspergillus niger
MVQIKAAALAVLFASNVLANPIEPRQASVSIDAKFKAHGKKYLGTIGDQYTLNKNAKTPAIIKADFGQLTPENSMKWDATEPNRGQFSFSGSDYLVNFAQSNGKLIRGHTLVWHSQLPSWVQSIYDKGTLIQVMQNHIATVMQRYKGKVYAWDVVNEIFNEDGSLRQSHFYNVIGEDYVRIAFETARAVDPNAKLYINDYNLDSASYPKLTGLVNHVKKWVAAGVPIDGIGSQTHLSAGAGAAVSGALNALAGAGTKEVAITELDIAGASSTDYVNVVKACLNQPKCVGITVWGVADPDSWRSSSSPLLFDSNYNPKAAYTAIANAL

SEQ ID NO: 34 - protein, 229 aa, Trichoderma reesi
MVAFSSLICALTSIASTLAMPTGLEPESSVNVTERGMYDFVLGAHNDHRRRASINYDQNYQTGGQVSYSPSNTGFSVNWNTQDDFVVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 35 - protein, 291 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 36 - protein, 645 aa, Talaromyces islandicus
MACLFYSSCALALALGQSALAAPNLSPRATVSLGSWLATETAVSLNGILDNIGASGAYAQSASAGIVIASPSTSNPNYYYTWTRDSALTLKVLIDVFRNGNLSLQTVIEEYVDAQAHLQTVTNPSGDLSEGAGLGEPKFNVDMSAFTDSWGRPQRDGPALRAIALIDFGNWLIENGYSTYAVTNIWPIVRNDLSYVAEYWSQSGYDLWEEVNSMSFFTVANQHRALVEGSTFAGKVGASCSWCDSQAPQILCYMQSFWTGSYINANTGGGRSGKDANTVLASIHTFDPEATCDDITFQPCSPRALANHKVYTDSFRSVYSLNSGIAEGVAVAVGRYPEDTYYNGNPWFLSNLAAAEQLYDAIYQWNKIGSISITSTSLAFFKDIYSSAAVGTYSSSTSTFTDIIDAVTTYADGYISIVQAHAMTNGSLSEQYDKSSGTELSARDLTWSYAAVLTANMRRSGIVPPSWGATSANTVPSSCSTGSATGTYSTPTATSWPSSLTSGTGATSTSTSTSTASVSSANHSTTTTTKATTTSSSCSNPTSVAVTFDEIATTSYGENIYIVGSISQLGDWDTDSAIQLSASKYTTSNHLWYATISLPAGTSFEYKYIRKESDGSITWESDPNRSYTVPLECAVTTATESDTWR

SEQ ID NO: 37 - protein, 1652 aa, Streptococcus pyogenes
MPKKKRKVGRVCRISSLRYRGPGIATMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSNGPTDAAEEELLSKNYHLENEVARLKKGSGSGGSGSGGSGSGGSGSGGSGGSGSGGSGSGGSGSGGSGSGEELLSKNYHLENEVARLKKGSGSGGSGSGGSGSGGSGSGGSGGSGSGGSGSGGSGSGGSGSGEELLSKNYHLENEVARLKKGSGSGGSGSGGSGSGGSGSGGSGGSGSGGSGSGGSGSGGSGSGEELLSKNYHLENEVARLKK

SEQ ID NO: 38 - protein, 829 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELGGGSGGYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK